(19) United States
(12) Patent Application Publication
(10) Pub. No.: US 2024/0130969 A1
(43) Pub. Date: Apr. 25, 2024

(54) LIPID NANOPARTICLES AND METHODS OF USE THEREOF

(71) Applicant: ReNAgade Therapeutics Management Inc., Cambridge, MA (US)

(72) Inventors: Muthusamy Jayaraman, Walpole, MA (US); Ciaran Lawlor, Cambridge, MA (US); Brian Goodman, Cambridge, MA (US)

(21) Appl. No.: 18/521,172

(22) Filed: Nov. 28, 2023

Related U.S. Application Data
(63) Continuation of application No. PCT/US2022/031383, filed on May 27, 2022.
(60) Provisional application No. 63/194,498, filed on May 28, 2021.

(51) Int. Cl. A61K 9/127; A61K 9/51; A61K 31/7088

(52) U.S. Cl. CPC A61K 9/1271; A61K 9/5123; A61K 9/5146; A61K 31/7088

(57) ABSTRACT

The present disclosure provides pharmaceutical compositions comprising lipid nanoparticles capable of delivering polynucleotide payloads to target non-liver organs.

### INCORPORATION BY REFERENCE OF SEQUENCE LISTING

The content of the electronically submitted sequence listing (Name: 4939_015PC01_Seglisting_ST25; Size: 3,617 bytes; and Date of Creation: May 26, 2022) filed with the application is incorporated herein by reference in its entirety.

### BACKGROUND

The effective targeted delivery of therapeutic nucleic acids has been a continuing medical challenge. Some pharmaceutical compositions, such as lipid nanoparticles, have been found to effectively provide protection to the nucleic acid to allow the nucleic acid to more safely reach a cell. However, getting the nucleic acid to reach a specific target organ continues to present a challenge. Typically, the nucleic acid will be delivered to the liver, but for some medical conditions, it would be better if it was delivered outside of the liver.

### SUMMARY

In an aspect of the present disclosure, provided herein is a pharmaceutical composition formulated for substantial extrahepatic delivery comprising:
a. a lipid nanoparticle comprising an ionizable lipid; and
b. a polynucleotide.

In another aspect, the present disclosure provides a pharmaceutical composition formulated for substantial extrahepatic delivery comprising:
a. a lipid nanoparticle comprising at least one ionizable lipid; and
b. a polynucleotide;
wherein the lipid nanoparticle encapsulates at least a portion of the polynucleotide and wherein the at least one ionizable lipid is selected from:
   i)

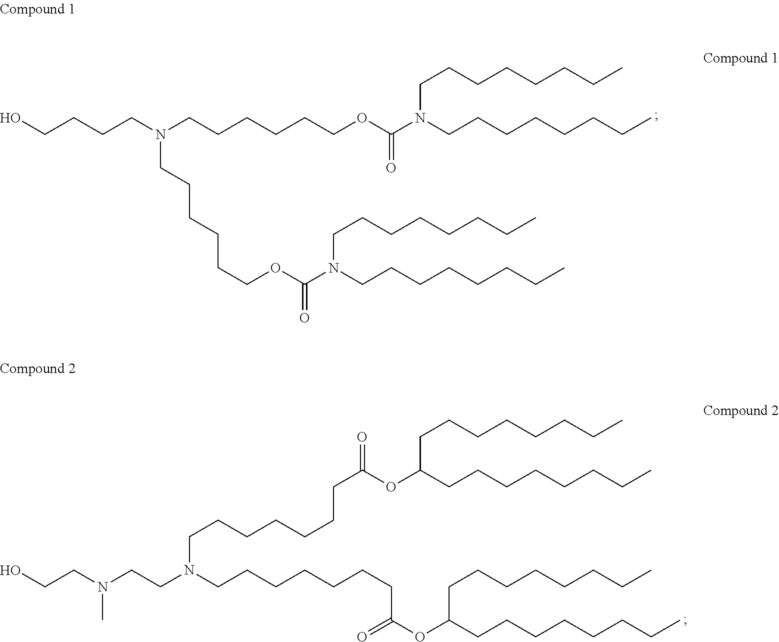 [SMILES: CCCCCCCCN(CCCCCCCC)C(=O)OCCCCCCN(CCCCO)CCCCCCOC(=O)N(CCCCCCCC)CCCCCCCC ; CCCCCCCCC(CCCCCCCC)OC(=O)CCCCCCCN(CCCCCCCC(=O)OC(CCCCCCCC)CCCCCCCC)CCN(C)CCO]

ii)
and
iii) an ionizable lipid disclosed in patent application publications WO2019/152557; WO2019/232095; WO2021/077067; WO2019/089828; US2019/0240354; US2010/0130588; US2021/0087135; US2021/0128488; US2020/0121809; US2013/0108685; US2013/0195920; US2015/0005363; US2014/0308304; US2017/0210697; and US2013/0053572.

In some embodiments, the lipid nanoparticle comprises Compound 1. In some embodiments, the lipid nanoparticle comprises Compound 2. In some embodiments, the lipid nanoparticle comprises an ionizable lipid disclosed in patent application publications WO2019/152557; WO2019/232095; WO2021/077067; WO2019/089828; US2019/0240354; US2010/0130588; US2021/0087135; US2021/0128488; US2020/0121809; US2013/0108685; US2013/0195920; US2015/0005363; US2014/0308304; US2017/0210697; and US2013/0053572.

In an aspect, at least 5%, 10%, 15%, 20%, 25%, 30%, 40%, 45%, 50%, 55%, 60%, 65%, 70%, 75%, 80%, 85%, 90%, or 95% of payload delivery occurs in a target organ.

In an aspect, the lipid nanoparticle delivers a higher proportion of its polynucleotide payload to a target organ than to the liver. In another aspect, the lipid nanoparticle delivers a higher proportion of the polynucleotide payload to the target organ than to the liver when administered to a mammalian subject.

In an aspect, the lipid nanoparticle delivers more of its polynucleotide payload to a target organ than a reference lipid nanoparticle does. In another aspect, the lipid nanoparticle delivers a higher proportion of the polynucleotide payload to a target organ than a reference lipid nanoparticle does when administered to a mammalian subject. In some embodiments, the reference lipid nanoparticle comprises at least one ionizable lipid selected from Compound 3 and Compound 4.

In an aspect, the reference lipid nanoparticle comprises MC3.

In an aspect, wherein the lipid portion of the reference lipid nanoparticle comprises about 50 mol % MC3, about 10 mol % DSPC, about 38.5 mol % cholesterol, and about 1.5 mol % PEG-DMG.

In an aspect, the ionizable lipid has a structure according to any of formulas 1-6.

In an aspect, the ionizable lipid has a head group listed on Table 1.

In an aspect, the ionizable lipid has a head group that contains a short peptide of 12-15 mer length.

In an aspect, the ionizable lipid has a head group that contains the structure of Vitamin A, D, E, or K.

In an aspect, the ionizable lipid has an alkyl tail.

In an aspect, the ionizable lipid has a disulfide tail.

In an aspect, the ionizable lipid contains an ester.

In an aspect, the ionizable lipid contains 1, 2, 3, or more branches.

In an aspect, the ionizable lipid has asymmetrical tails.

In an aspect, the ionizable lipid has a pKa between 6 and 7.

In an aspect, the ionizable lipid is positively charged.

In an aspect, the target organ is the lung.

In an aspect, the lipid nanoparticle further comprises a PEGylated lipid.

In an aspect, the PEGylated lipid is PEG-DMG. In an aspect, the PEGylated lipid is PEG-c-DOMG, PEG-DMG, PEG-DLPE, PEG-DMPE, PEG-DPPC, or PEG-DSPE. In an aspect, the PEGylated lipid is PEG-DSPE.

In an aspect, the lipid nanoparticle further comprises a structural lipid.

In an aspect, the structural lipid is cholesterol.

In an aspect, the cholesterol is replaced with a cholesterol analog.

In an aspect, the structural lipid contains a plant sterol mimetic.

In an aspect, the lipid nanoparticle further comprises a phospholipid.

In an aspect, the phospholipid is modified for enhanced endosomal escape.

In an aspect, the phospholipid is selected from DOPE and DSPC.

In another aspect, the lipid nanoparticle further comprises at least one lipid selected from DDAB, EPC, 14PA, 18BMP, DODAP, DOTAP, and C12-200.

In an aspect, the polynucleotide is DNA.

In an aspect, the polynucleotide is RNA.

In an aspect, the RNA is circular RNA.

In an aspect, the RNA is a short interfering RNA (siRNA), an asymmetrical interfering RNA (aiRNA), a microRNA (miRNA), a Dicer-substrate RNA (dsRNA), or a short hairpin RNA (shRNA).

In an aspect, the RNA consists of fewer than about 15, 20, 25, 30, or 50 nucleotides.

In an aspect, the polynucleotide encodes a protein.

In an aspect, the polynucleotide comprises at least about 15, 20, 25, 30, 50, 100, 500, 1000, 1500, 2000, 2500, 3000, 4000, 5000, 6000, 7000, 8000, 9000, 10000, or greater than 10000 nucleotides.

In an aspect, the polynucleotide has been modified by a glycan.

In an aspect, the polynucleotide consists of natural nucleotides.

In an aspect, a pharmaceutical composition is formulated for systemic administration to a human subject in need thereof.

In an aspect, a pharmaceutical composition is formulated for administration into a target organ in a human subject in need thereof.

In an aspect, the target organ is the kidney, placenta, heart, lung, muscle, fat, bladder, spleen, adrenal glands, brain, vagina, immune system, central nervous system, or skin.

In an aspect, at least 5%, 10%, 15%, 20%, 25%, 30%, 40%, 45%, 50%, 55%, 60%, 65%, 70%, 75%, 80%, 85%, 90%, or 95% of polynucleotides are encapsulated within lipid nanoparticles.

In an aspect, a pharmaceutical composition further comprises a target organ binding moiety.

In an aspect, the target organ binding moiety is operably connected to the lipid nanoparticle.

In some embodiments, when the pharmaceutical composition is administered to a mammalian subject, at least about 5%, about 10%, about 15%, or about 20% of polynucleotide delivery occurs in a target organ.

In an aspect, provided herein is a method of treating a subject in need thereof, comprising administering to the subject a pharmaceutical composition described herein.

In an aspect, provided herein is a method of treating a subject in need thereof, comprising administering to the subject a pharmaceutical composition described herein through systemic administration.

In an aspect, provided herein is a method of treating a subject in need thereof, comprising administering to the subject the pharmaceutical composition described herein through local administration.

### BRIEF DESCRIPTION OF THE DRAWINGS

FIGS. 1A-1E are in vivo whole body bioluminescence images of mice dosed with fLuc mRNA formulated with LNP formulations F-1 (FIG. 1A), F-2 (FIG. 1B), F-3 (FIG. 1C), F-4 (FIG. 1D) and PBS control (FIG. 1E), 6 hours and 24 hours post injection.

FIGS. 2A-2J are bioluminescence images of livers, spleens, lungs, hearts and kidneys of mice dosed with fLuc mRNA formulated with LNP formulations F-1 (FIG. 2A and FIG. 2B), F-2 (FIG. 2C and FIG. 2D), F-3 (FIG. 2E and FIG. 2F), F-4 (FIG. 2G and FIG. 2H) and PBS control (FIG. 2I and FIG. 2J), 6 hours and 24 hours after administration.

FIGS. 3A and 3B are bar graphs showing relative distribution (FIG. 3A) and absolute luminescence (FIG. 3B) of fLuc mRNA in liver and spleen of mice dosed with fLuc mRNA formulated within LNP formulations F-1, F-2, F-3, F-4 and PBS control, 6 hours and 24 hours after administration.

FIG. 4 is a bar graph showing absolute luminescence of fLuc mRNA in liver and spleen of mice dosed with fLuc mRNA formulated within LNP formulations F-1 and F-3, 6 hours after administration.

FIGS. 5A-5C are bar graphs showing GFP expression by flow cytometry in red pulp macrophages (FIG. 5A), CD11b+IA/IE+ myeloid cells (FIG. 5B), and dendritic cells (FIG. 5C), 1 hour after administration. Gating schemes used to identify populations are as follows: red pulp macrophages (FIG. 5A) Cells→Singlets→Live→CD45+→CD19− CD3−→CD11b−→F4/80+ IA/IE+; CD11b+ IA/IE+ myeloid cells (FIG. 5B) Cells→Singlets→Live→CD45+→CD19− CD3− [symbol] CD11b+ [symbol] IA/IE+; Cells [symbol] dendritic cells (FIG. 5C) Singlets [symbol] Live [symbol] CD45+ [symbol] CD19− CD3− [symbol] CD11b+ [symbol] IA/IE+ CD11c+.

### DETAILED DESCRIPTION

The present disclosure relates to pharmaceutical compositions comprising lipid nanoparticles (LNP). The lipid nanoparticles of the present disclosure are capable of delivering a payload to a target organ or cell population, preferably a non-liver target organ or cell population. Preferably, the payload is a polynucleotide.

In some embodiments, a lipid nanoparticle comprises an ionizable lipid, a structural lipid, a PEGylated lipid, and a phospholipid. The specific lipids and formulation chosen may be optimized for enhanced delivery to a target organ or cell population. For example, the ionizability, pKa, and hydrophobicity of the compounds can be optimized for increased delivery to a target organ or cell population. In some embodiments, the head, linker, or tails of an ionizable lipid may be chosen to optimize target organ or cell population delivery. In some embodiments, a pharmaceutical composition further comprises a binding moiety capable of directing a nanoparticle to a target organ or cell population. In some embodiments, the binding moiety is operably connected to a lipid nanoparticle. In some embodiments, a target organ or cell population is the kidney, placenta, heart, lung, muscle, fat, bladder, spleen, adrenal glands, brain, vagina, immune system, central nervous system, or skin.

In some embodiments, a composition is optimized for reduced delivery to hepatic cells or the liver.

In some embodiments, at least about 5%, 10%, 15%, 20%, 25%, 30%, 35%, 40%, 45%, 50%, 55%, 60%, 65%, 70%, 75%, 80%, 85%, 90%, or 95% of the payload that is delivered to a cell is delivered to a cell of the target organ or cell population. In some embodiments, less than about 5%, 10%, 15%, 20%, 25%, 30%, 35%, 40%, 45%, 50%, 55%, 60%, 65%, 70%, 75%, 80%, 85%, 90%, or 95% of the payload that is delivered to a cell is delivered to the liver or hepatic cells.

In some embodiments, a polynucleotide encodes a therapeutic protein. In some embodiments, at least about 5%, 10%, 15%, 20%, 25%, 30%, 35%, 40%, 45%, 50%, 55%, 60%, 65%, 70%, 75%, 80%, 85%, 90%, or 95% of therapeutic protein expression occurs in a target organ or cell population. In some embodiments, less than about 5%, 10%, 15%, 20%, 25%, 30%, 35%, 40% 45%, 50%, 55%, 60%, 65%, 70%, 75%, 80%, 85%, 90%, or 95% of therapeutic protein>expression occurs in the liver or hepatic cells.

Described herein are compositions and methods for delivery of polynucleotide moieties for extrahepatic delivery. The disclosure provides various types of lipids that can be used to deliver polynucleotides to target organs other than the liver. The disclosure also provides methods of delivering a pharmaceutical composition to a target organ, target cell, or target tissue in need thereof.

### A. Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Alkyl” refers to a straight or branched hydrocarbon chain radical consisting solely of carbon and hydrogen atoms, which is saturated or unsaturated (i.e., contains one or more double and/or triple bonds), having from one to thirty or more carbon atoms (e.g., C₁-C₂₄ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, propyl enyl, but-1-enyl, pent-1-enyl, penta-1,4-dienyl, ethynyl, propynyl, butynyl, pentynyl, hexynyl, and the like. Alkyl groups that include one or more units of unsaturation (one or more double and/or triple bond) can be C₂-C₂₄, C₂-C₁₂, C₂-C₈ or C₂-C₆ groups, for example. Unless specifically stated otherwise, an alkyl group is optionally substituted. The term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

“Alkylene” or “alkylene chain” refers to a straight or branched divalent hydrocarbon chain consisting solely of carbon and hydrogen, which is saturated or unsaturated (i.e., contains one or more double (alkenylene) and/or triple bonds (alkynylene)), and having, for example, from one to thirty or more carbon atoms (e.g., C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. Alkylene groups that include one or more units of unsaturation (one or more double and/or triple bond) can be C₂-C₂₄, C₂-C₁₂, C₂-C₈ or C₂-C₆ groups, for example. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

As used herein, the term “heteroalkyl” by itself or in combination with another term means, unless otherwise stated, a stable straight or branched chain alkyl group consisting of the stated number of carbon atoms and one or two or more heteroatoms typically selected from the group consisting of O, N, Si, P, and S, and wherein the nitrogen and sulfur atoms may be optionally oxidized and the nitrogen heteroatom may be a primary, secondary, tertiary or quaternary nitrogen. The heteroatom(s) may be placed at any position of the heteroalkyl group, including between the rest of the heteroalkyl group and the fragment to which it is attached, as well as attached to the most distal carbon atom in the heteroalkyl group. Examples of heteroalkyl groups include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

“Heterocyclyl” or “heterocyclic ring” refers to a stable 3- to 18-membered non-aromatic ring radical which consists of two to twelve carbon atoms and from one to six heteroatoms typically selected from the group consisting of N, O, Si, P, and S. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

As used herein, the term “aromatic” refers to a carbocycle or heterocycle with one or more polyunsaturated rings and having aromatic character, i.e. having (4n+2) delocalized p (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heteroaryl” or “heteroaromatic” refers to aryl groups which contain at least one heteroatom typically selected from N, O, Si, P, and S; wherein the nitrogen and sulfur atoms may be optionally oxidized, and the nitrogen atom(s) may be optionally teriatry or quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide. Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl. The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, quaternary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the terms “alkoxy,” “alkylamino” and “alkylthio” are used in their conventional sense, and refer to alkyl groups linked to molecules via an oxygen atom, an amino group, a sulfur atom, respectively.

For example, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, a “lipid nanoparticle” or “LNP” is a composition comprising one or more lipids. LNPs are typically sized on the order of micrometers or smaller and may include a lipid bilayer, and preferably has an average size of less than 1 micrometer.

As used herein, “nucleic acid” is meant to include any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). The term “nucleic acid” typically refers to large polynucleotides.

As used herein, a “PEG lipid” or “PEGylated lipid” refers to a lipid comprising a polyethylene glycol component.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment, which has been separated from sequences which flank it in a naturally occurring state, i.e., a DNA fragment, which has been removed from the sequences which are normally adjacent to the fragment, i.e., the sequences adjacent to the fragment in a genome in which it naturally occurs. The term also applies to nucleic acids which have been substantially purified from other components, which naturally accompany the nucleic acid, i.e., RNA or DNA or proteins, which naturally accompany it in the cell. The term therefore includes, for example, a recombinant DNA or RNA, which is incorporated into a vector, into an autonomously replicating plasmid or virus, or into the genomic DNA or RNA of a prokaryote or eukaryote, or which exists as a separate molecule (i.e., as a cDNA or a genomic or cDNA fragment produced by PCR or restriction enzyme digestion) independent of other sequences. It also includes a recombinant DNA or RNA, which is part of a hybrid gene encoding additional polypeptide sequence.

The term “DNA” is a well-known term of art that refers to deoxyribonucleic acid.

The term “RNA” is a well-known term of art that refers to ribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “identical” refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

An “effective amount” as used herein, means an amount which provides a therapeutic or prophylactic benefit under the conditions of administration.

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

To “treat” a disease as the term is used herein, means to reduce the frequency or severity of at least one sign or symptom of a disease or disorder experienced by a subject.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of polynucleotide are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, the following abbreviations and initialisms have the indicated meanings:

| | |
|---|---|
| MC3 | 4-(dimethylamino)-butanoic acid, |
| | (10Z,13Z)-1-(9Z,12Z)-9,12-octadecadien- |
| | 1-yl-10,13-nonadecadien-1-yl ester |
| DSPC | 1,2-distearoyl-sn-glycero-3-phosphocholine |
| DMG | 1,2-Dimyristoyl-rac-glycero-3-methanol |
| DOMG-PEG | R-3-[(ω-methoxy-poly(ethyleneglycol))carbamoyl)]- |
| | 1,2-dimyristyloxypropyl-3-amine |
| DLPE | 1,2-Dilauroyl-sn-Glycero-3-Phosphoethanolamine |
| DMPE | 1,2-Dimyristoyl-sn-Glycero-3-Phosphoethanolamine |
| DPPC | 1,2-dipalmitoyl-sn-glycero-3-phosphocholine |
| DSPE | 1,2-distearoyl-sn-glycero-3-phosphoethanolamine |
| DDAB | Didodecyldimethylammonium bromide |
| EPC | 1,2-dioleoyl-sn-glycero-3-ethylphosphocholine |
| 14PA | 1,2-dimyristoyl-sn-glycero-3-phosphate |
| 18BMP | bis(monooleoylglycero)phosphate |
| DODAP | 1,2-dioleoyl-3-dimethylammonium-propane |
| DOTAP | 1,2-dioleoyl-3-trimethylammonium-propane |
| C12-200 | 1,1′-((2-(4-(2-((2-(bis(2- |
| | hydroxydodecyl)amino)ethyl)(2- |
| | hydroxydodecyl)amino)ethyl)piperazin-1- |
| | yl)ethyl)azanediyl)bis(dodecan-2-ol) |

### B. Lipid Nanoparticle Compositions

In some embodiments, a LNP comprises an ionizable lipid, a structural lipid, a PEGylated lipid, and a phospholipid. In some embodiments, an LNP further comprises a 5^{th} lipid. In some embodiments, a LNP further comprises a targeting moiety. In preferred embodiments, the LNP encapsulates a polynucleotide.

In some embodiments, a LNP has a diameter of at least about 50 nm, 60 nm, 70 nm, 80 nm, or 90 nm. In some embodiments, a LNP has a diameter of less than about 110 nm, 120 nm, 130 nm, 140 nm, 150 nm, or 160 nm. In some embodiments, a LNP has a diameter of about 100 nm.

In some embodiments, the lipid compositions described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG modified lipid may be from about 0.1 mol-% to about 10 mol-%. In some embodiments, the mol-% of the PEG modified lipid may be from about 0.2 mol-% to about 5 mol-%. In some embodiments, the mol-% of the PEG modified lipid may be from about 0.5 mol-% to about 3 mol-%. In some embodiments, the mol-% of the PEG modified lipid may be from about 1 mol-% to about 2 mol-%. In some embodiments, the mol-% of the PEG modified lipid may be about 1.5 mol-%.

i. Ionizable Lipids

In some embodiments, a LNP contains an ionizable lipid.

In some embodiments, an ionizable lipid has a dimethylamine or an ethanolamine head. In some embodiments, an ionizable lipid has an alkyl tail. In some embodiments, a tail has one or more ester linkages, which may enhance biodegradability. In some embodiments, a tail is branched, such as with 3 or more branches. In some embodiments, a branched tail may enhance endosomal escape. In some embodiments, an ionizable lipid has a pKa between 6 and 7, which may be measured, for example, by TNS assay.

In some embodiments, an ionizable lipid comprises a head group from Table 1. In some embodiments, an ionizable lipid has a structure according to any of formulas 1-6 below. In some embodiments, an ionizable lipid has a structure of any of the formulas disclosed below, and all formulas disclosed in a reference publication and patent application publication cited below. In some embodiments, an ionizable lipid comprises a head group of any structure or formula disclosed below. In some embodiments, an ionizable lipid comprises a bridging moiety of any structure or formula disclosed below. In some embodiments, an ionizable lipid comprises any tail group, or combination of tail groups disclosed below. The present disclosure contemplates all permutations and combinations of head group, bridging moiety and tail group, or tail groups, disclosed herein.

TABLE 1
Ionizable lipid head groups

| Head number | Structure |
|---|---|
| 1 | 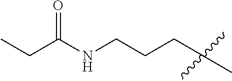 [SMILES: *CCCNC(=O)CC] |
| 2 | 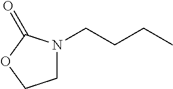 |
| 3 | 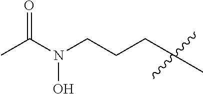 [SMILES: *CCCN(O)C(C)=O] |
| 4 | 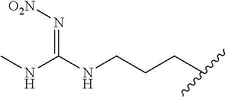 [SMILES: *CCCN/C(=N/[N+](=O)[O-])NC] |
| 5 | 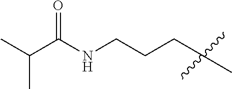 [SMILES: *CCCNC(=O)C(C)C] |
| 6 | 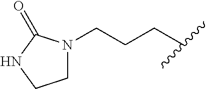 [SMILES: *CCCN1CCNC1=O] |
| 7 | 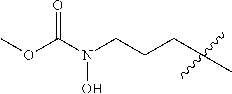 [SMILES: *CCCN(O)C(=O)OC] |
| 8 | 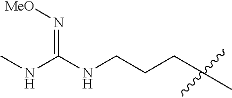 [SMILES: *CCCN/C(=N/OC)NC] |
| 9 | 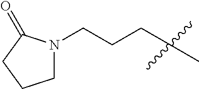 [SMILES: *CCCN1CCCC1=O] |
| 10 | 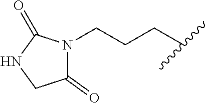 [SMILES: *CCCN1C(=O)CNC1=O] |
| 11 | 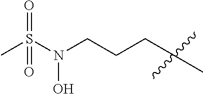 [SMILES: *CCCN(O)S(C)(=O)=O] |
| 12 | 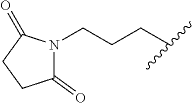 [SMILES: *CCCN1C(=O)CCC1=O] |
| 13 | 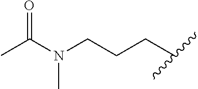 [SMILES: *CCCN(C)C(C)=O] |
| 14 | 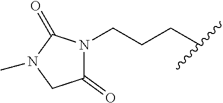 [SMILES: *CCCN1C(=O)CN(C)C1=O] |
| 15 | 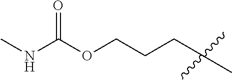 [SMILES: *CCCOC(=O)NC] |
| 16 | 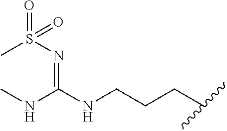 [SMILES: *CCCN/C(=N/S(C)(=O)=O)NC] |
| 17 | 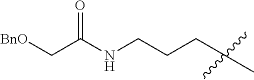 [SMILES: *CCCNC(=O)COCc1ccccc1] |
| 18 | 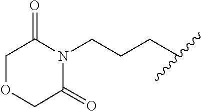 [SMILES: *CCCN1C(=O)COCC1=O] |
| 19 | 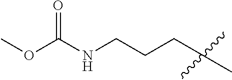 [SMILES: *CCCNC(=O)OC] |
| 20 | 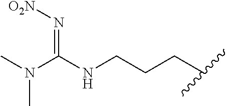 [SMILES: *CCCN/C(=N/[N+](=O)[O-])N(C)C] |
| 21 | 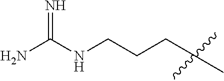 [SMILES: *CCCNC(=N)N] |
| 22 | 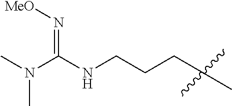 [SMILES: *CCCN/C(=N/OC)N(C)C] |
| 23 | 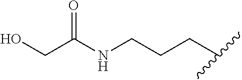 [SMILES: *CCCNC(=O)CO] |
| 24 | 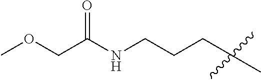 [SMILES: *CCCNC(=O)COC] |
| 25 | 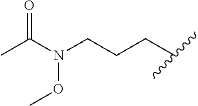 [SMILES: *CCCN(OC)C(C)=O] |
| 26 | 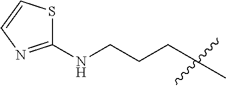 [SMILES: *CCCNc1nccs1] |
| 27 | 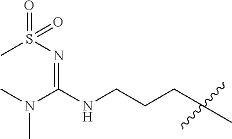 [SMILES: *CCCN/C(=N/S(C)(=O)=O)N(C)C] |
| 28 | 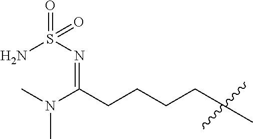 [SMILES: *CCCC/C(=N/S(N)(=O)=O)N(C)C] |
| 29 | 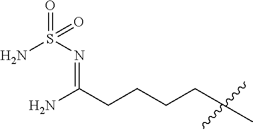 [SMILES: *CCCC/C(N)=N/S(N)(=O)=O] |
| 30 | 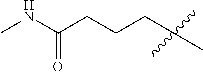 [SMILES: *CCCC(=O)NC] |
| 31 | 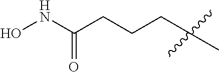 [SMILES: *CCCC(=O)NO] |
| 32 | 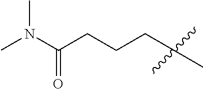 [SMILES: *CCCC(=O)N(C)C] |
| 33 | 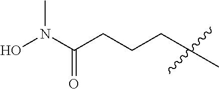 [SMILES: *CCCC(=O)N(C)O] |
| 34 | 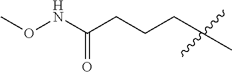 [SMILES: *CCCC(=O)NOC] |
| 35 | 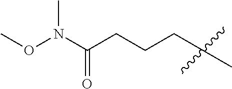 [SMILES: *CCCC(=O)N(C)OC] |
| 36 | 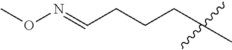 [SMILES: *CCC/C=N/OC] |
| 37 | 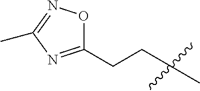 [SMILES: *CCc1nc(C)no1] |
| 38 | 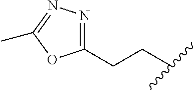 [SMILES: *CCc1nnc(C)o1] |
| 39 | 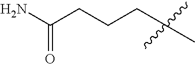 [SMILES: *CCCC(N)=O] |
| 40 | 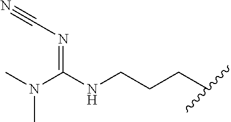 [SMILES: *CCCN/C(=N/C#N)N(C)C] |
| 41 | 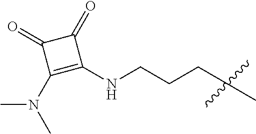 [SMILES: *CCCNc1c(N(C)C)c(=O)c1=O] |
| 42 | 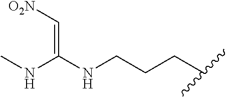 [SMILES: *CCCN/C(=C/[N+](=O)[O-])NC] |
| 43 | 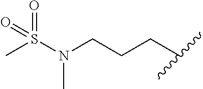 [SMILES: *CCCN(C)S(C)(=O)=O] |
| 44 | 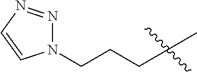 [SMILES: *CCCn1ccnn1] |
| 45 | 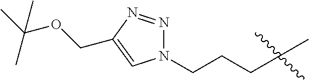 [SMILES: *CCCn1cc(COC(C)(C)C)nn1] |
| 46 | 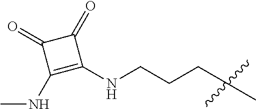 [SMILES: *CCCNc1c(NC)c(=O)c1=O] |
| 47 | 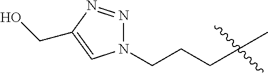 [SMILES: *CCCn1cc(CO)nn1] |
| 48 | 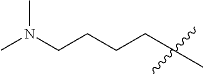 [SMILES: *CCCCN(C)C] |
| 49 | 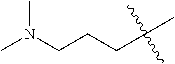 [SMILES: *CCCN(C)C] |
| 50 | 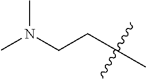 [SMILES: *CCN(C)C] |
| 51 | 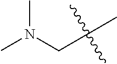 [SMILES: *CN(C)C] |
| 52 | 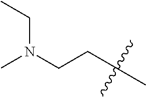 [SMILES: *CCN(C)CC] |
| 53 | 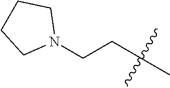 [SMILES: *CCN1CCCC1] |
| 54 | 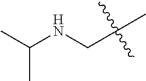 [SMILES: *CNC(C)C] |
| 55 | 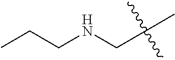 [SMILES: *CNCCC] |
| 56 | 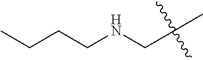 [SMILES: *CNCCCC] |
| 57 | 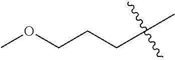 [SMILES: *CCCOC] |
| 58 | 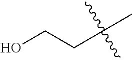 [SMILES: *CCO] |
| 59 | 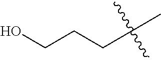 [SMILES: *CCCO] |
| 60 | 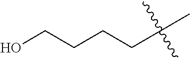 [SMILES: *CCCCO] |
| 61 | 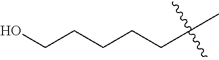 [SMILES: *CCCCCO] |
| 62 | 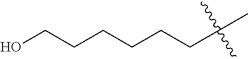 [SMILES: *CCCCCCO] |
| 63 | 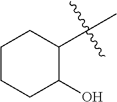 [SMILES: *C1CCCCC1O] |
| 64 | 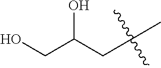 [SMILES: *CC(O)CO] |
| 65 | 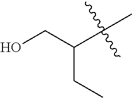 [SMILES: *C(CC)CO] |
| 66 | 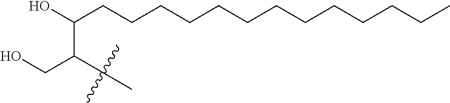 [SMILES: *C(CO)C(O)CCCCCCCCCCCCC] |
| 67 | 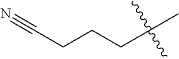 [SMILES: *CCCC#N] |
| 68 | 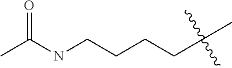 [SMILES: *CCCCNC(C)=O] |

In some embodiments, an ionizable lipid is described in international patent application WO2019152557, which is incorporated herein by reference in its entirety. In some embodiments, an ionizable lipid has one of the following head groups:

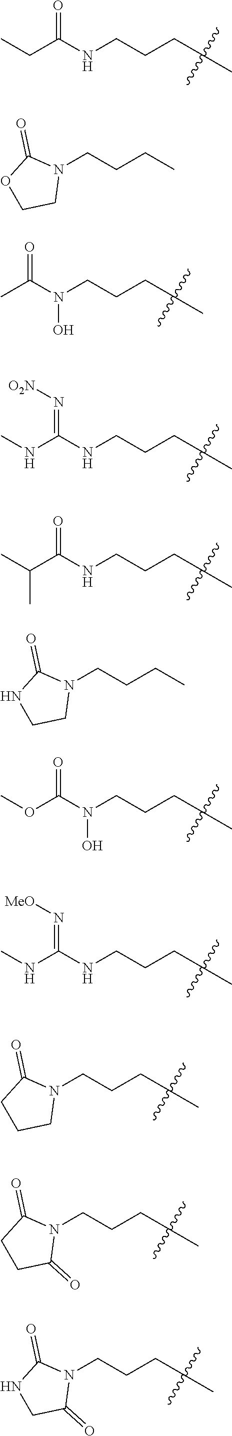

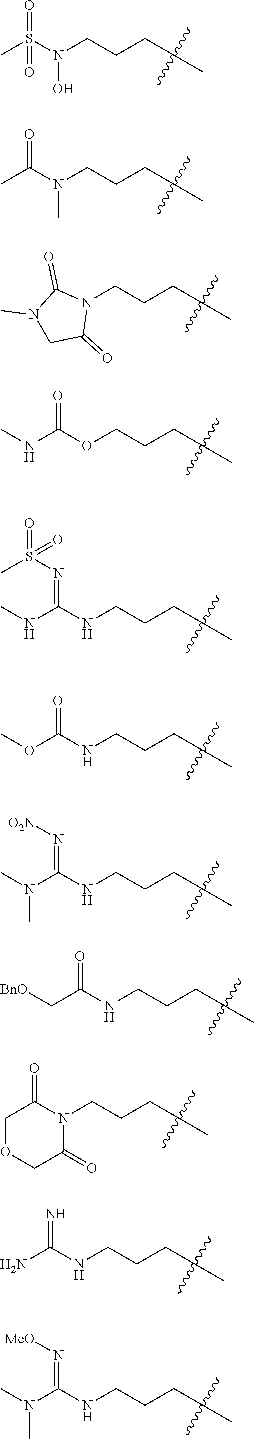

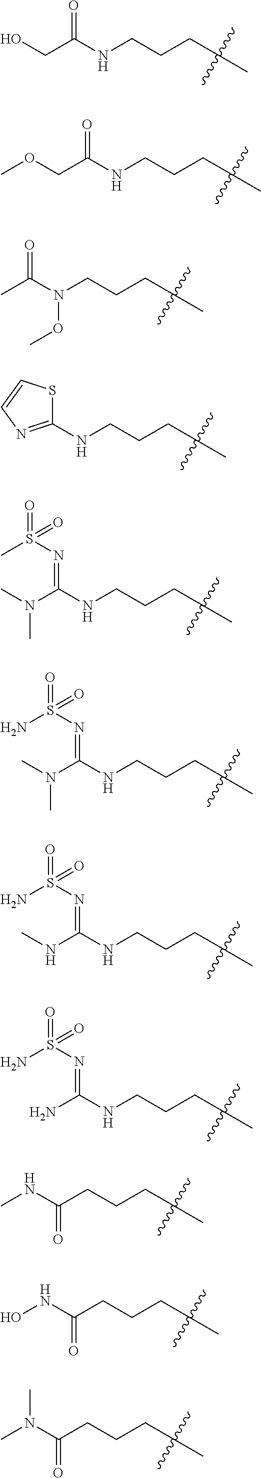

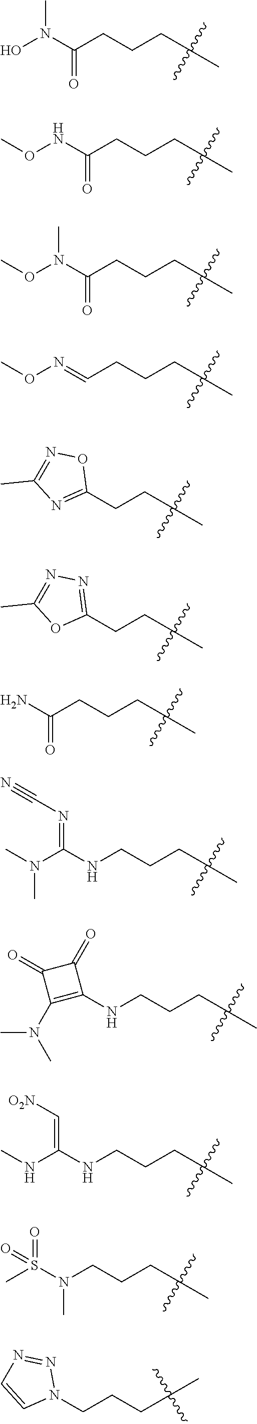

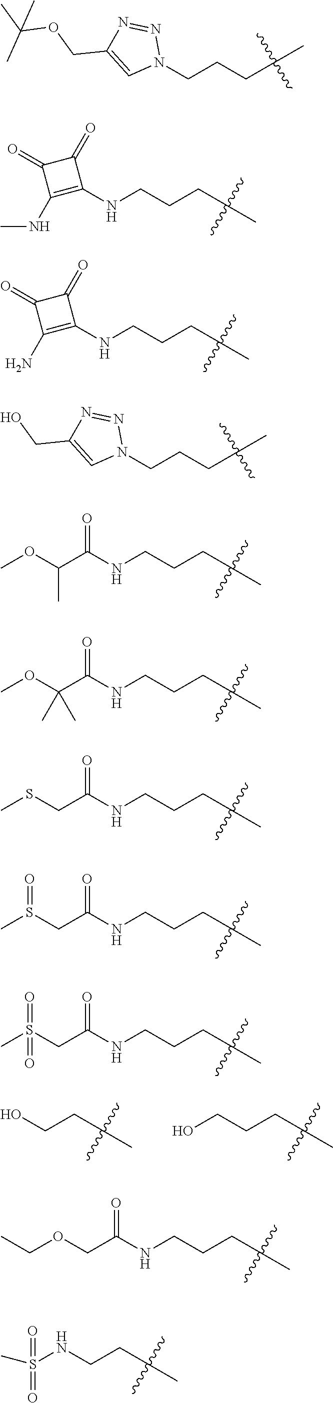

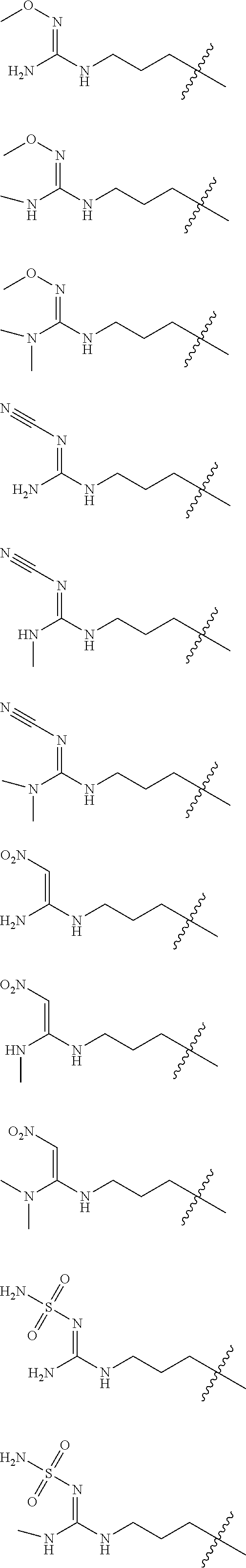

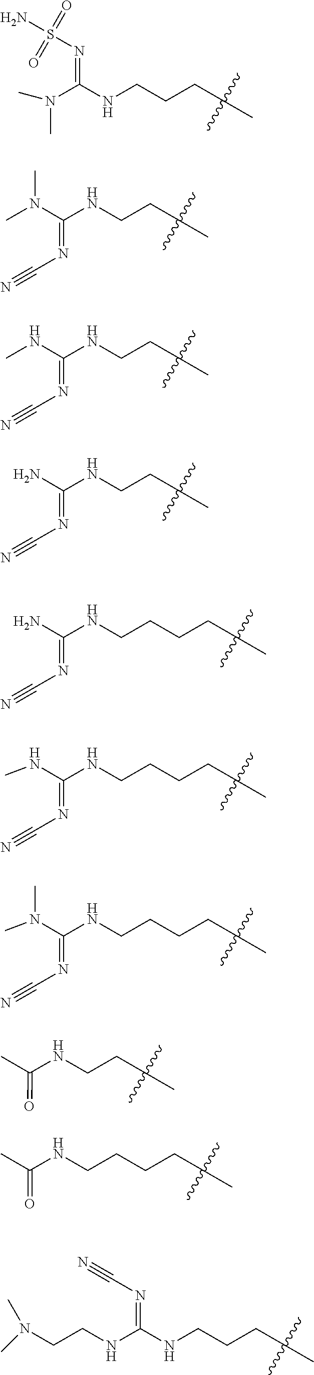

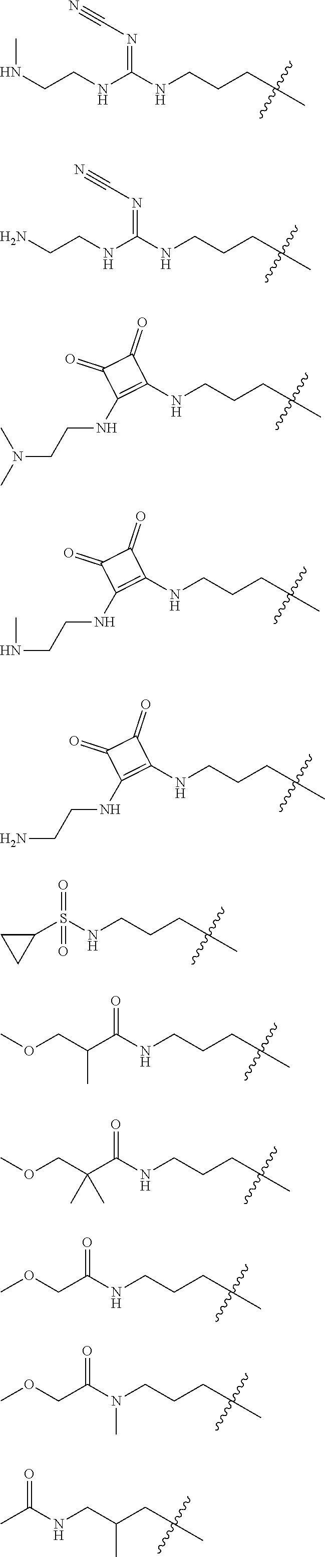

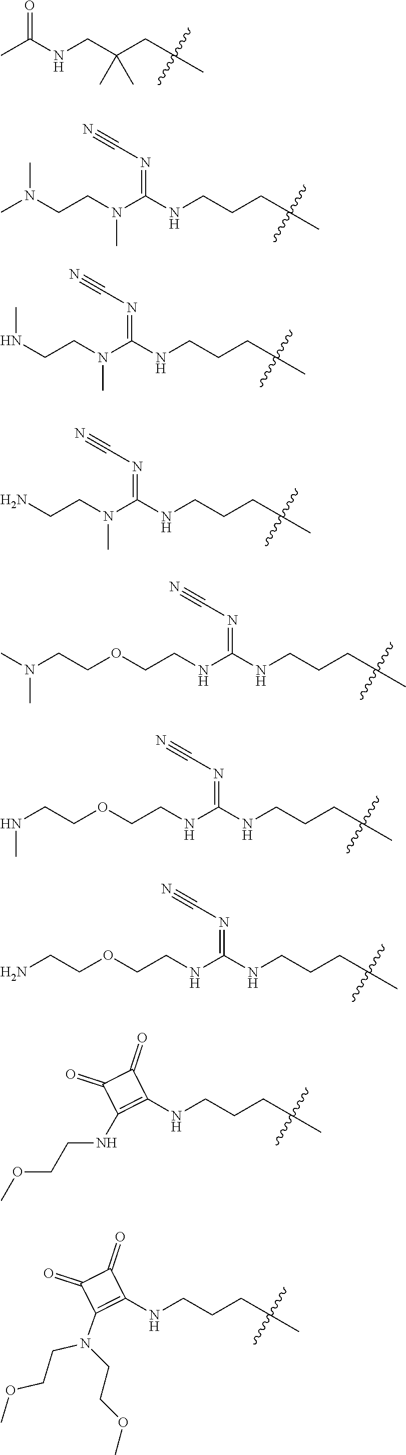

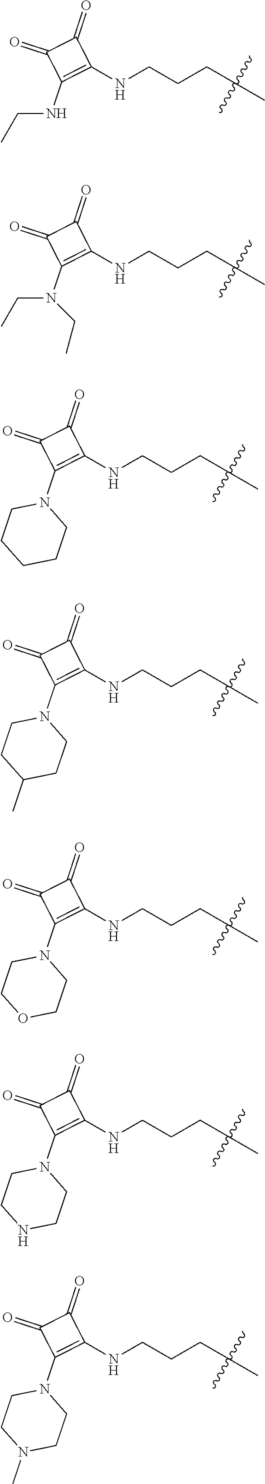

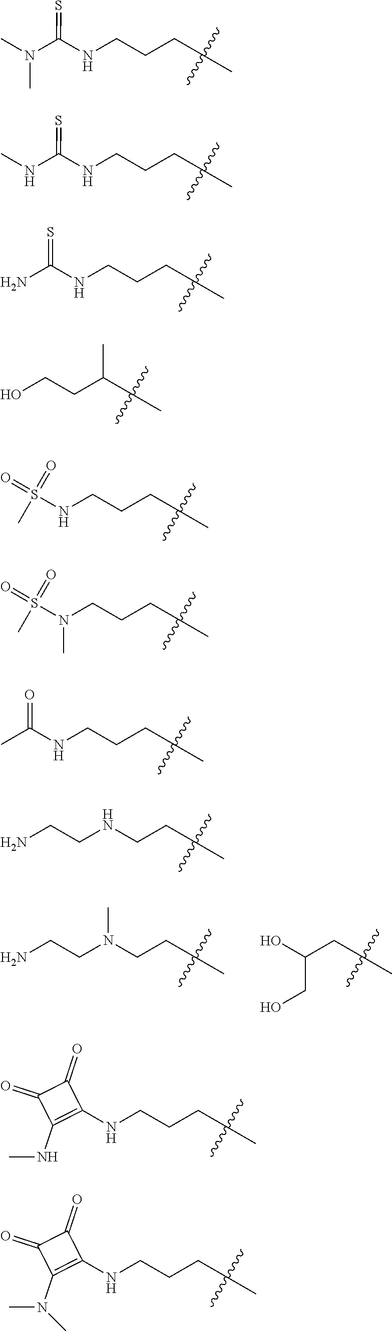

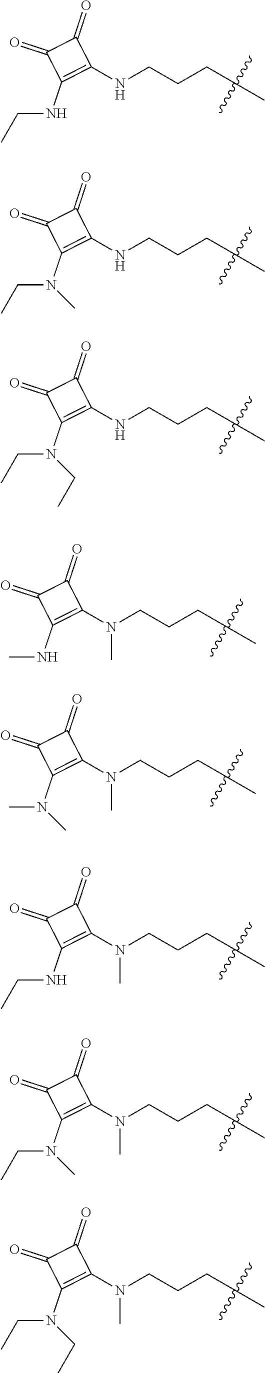

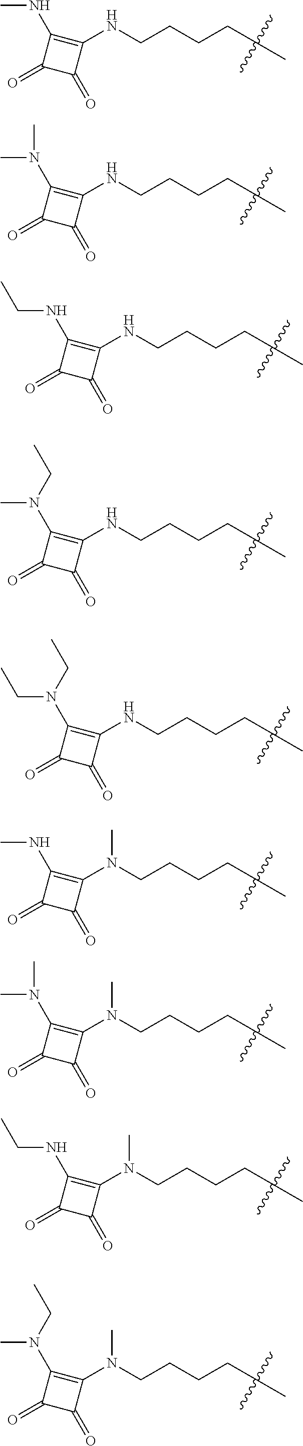

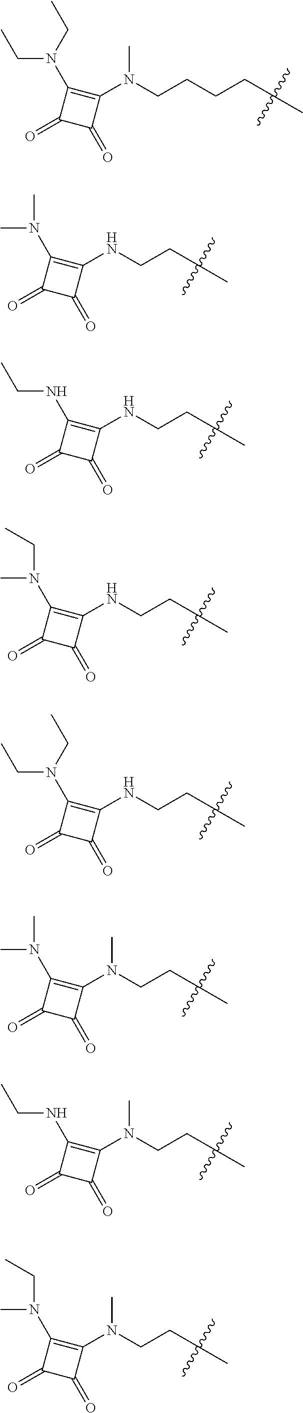

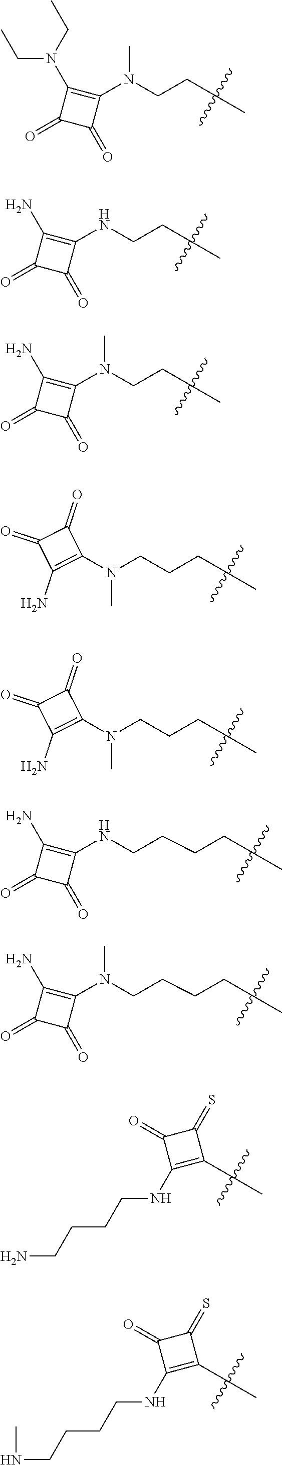

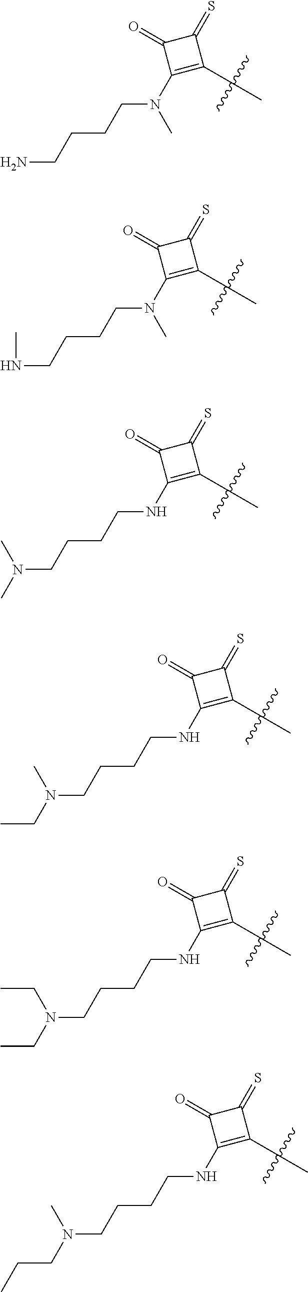 [SMILES: *c1c(N(C)CCCCN)c(=O)c1=S ; *c1c(N(C)CCCCNC)c(=O)c1=S ; *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S]

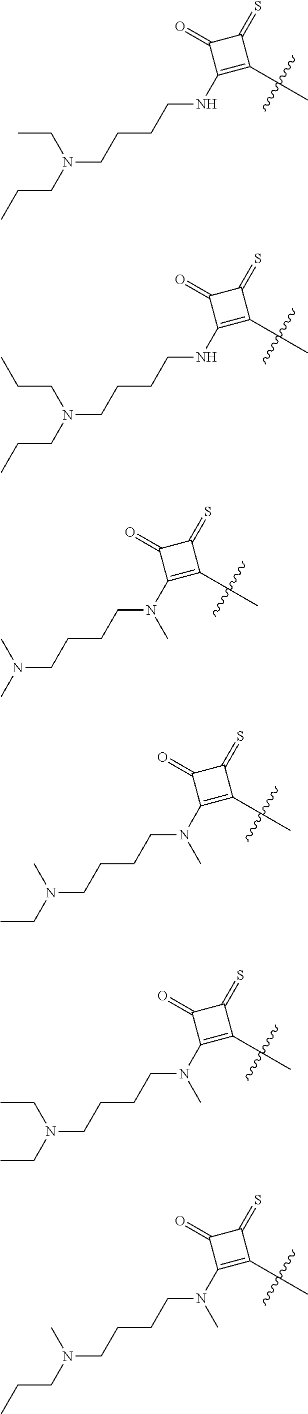 [SMILES: *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S]

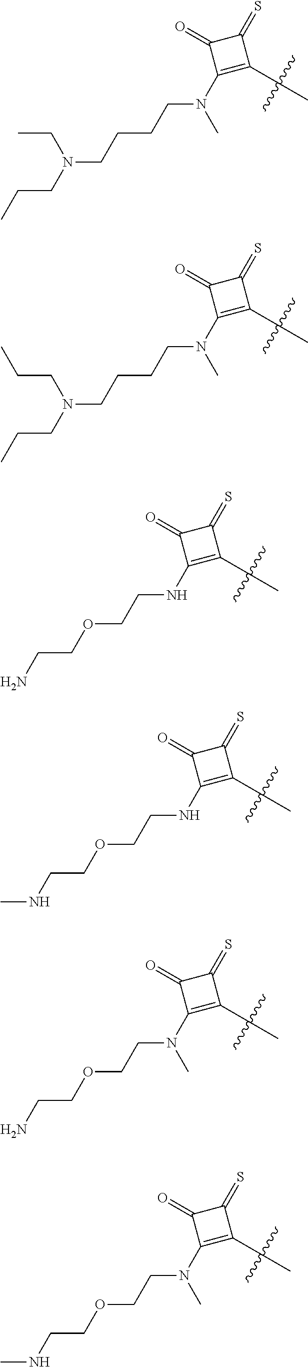 [SMILES: *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

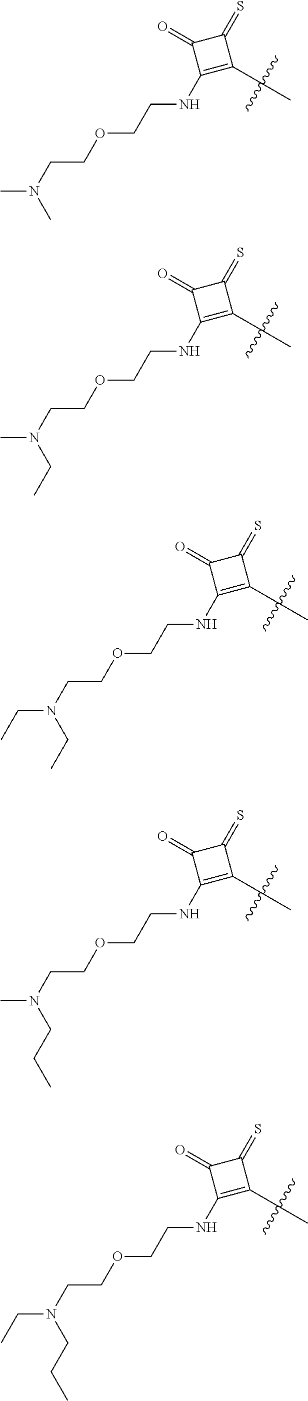 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

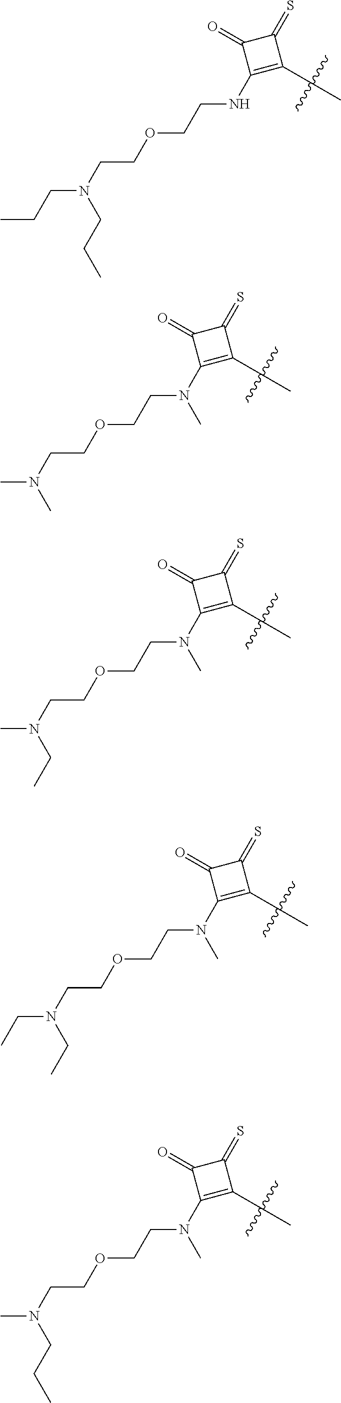 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

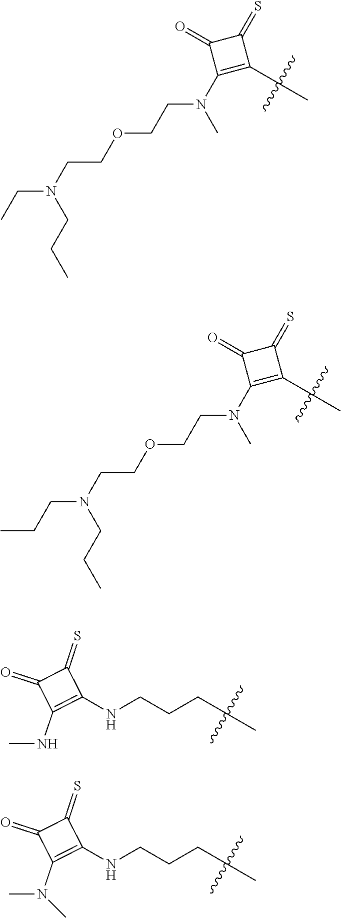 [SMILES: *c1c(N(C)CCOCCN(CC)CCC)c(=O)c1=S ; *c1c(N(C)CCOCCN(CCC)CCC)c(=O)c1=S ; *CCCNc1c(NC)c(=O)c1=S ; *CCCNc1c(N(C)C)c(=O)c1=S]

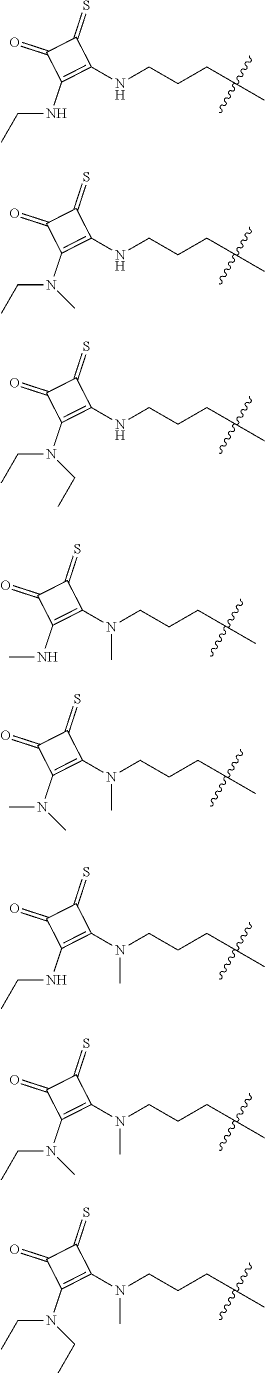

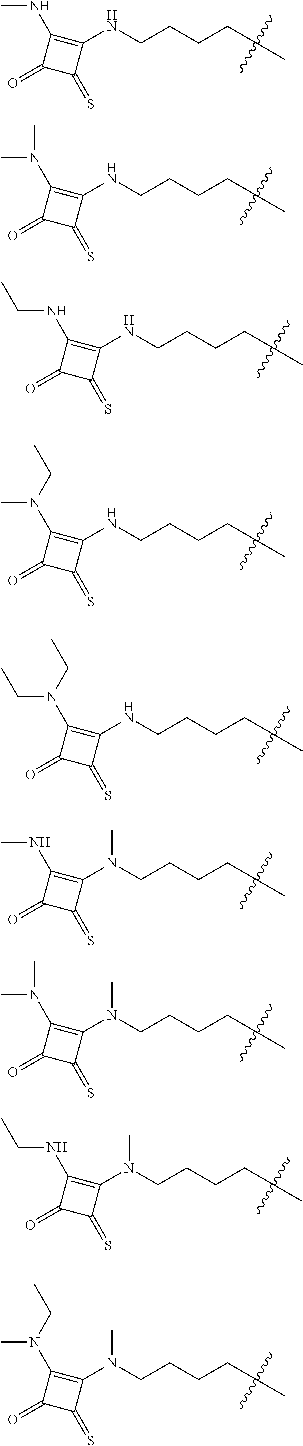

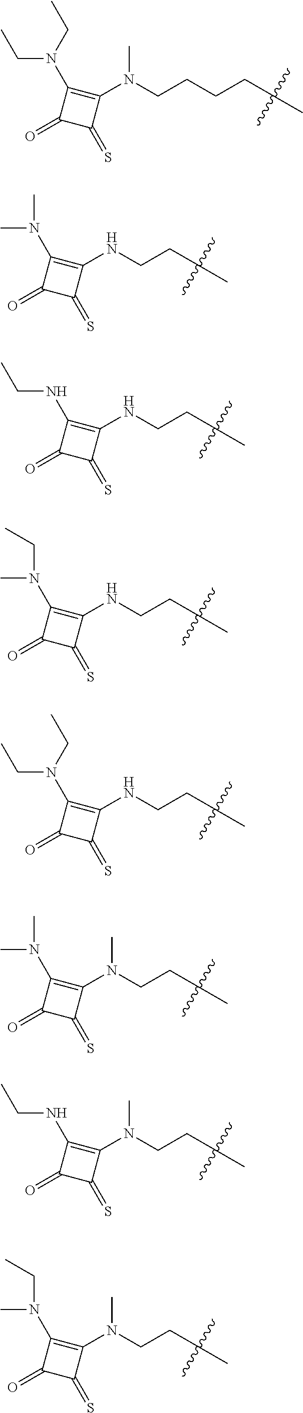

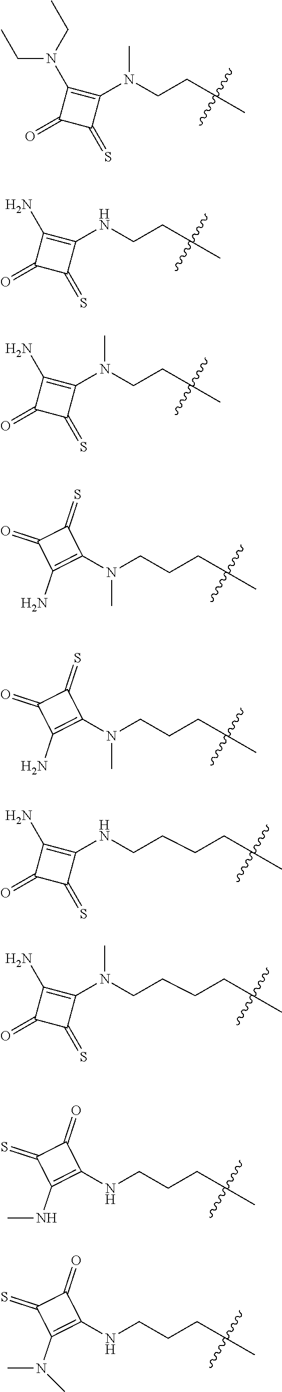

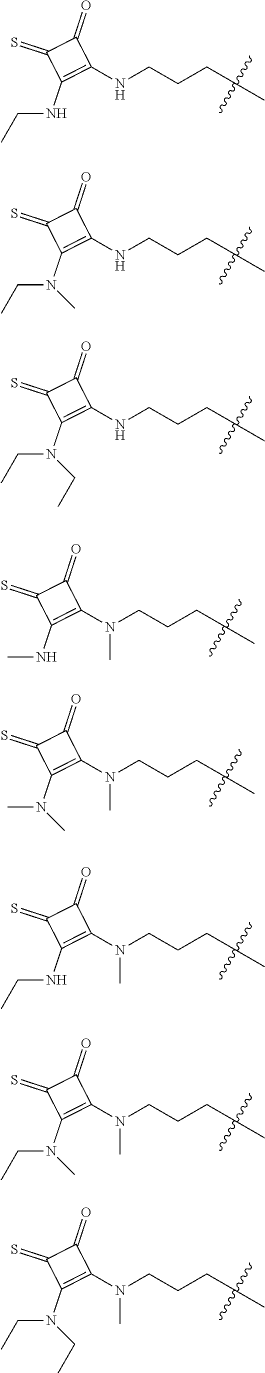

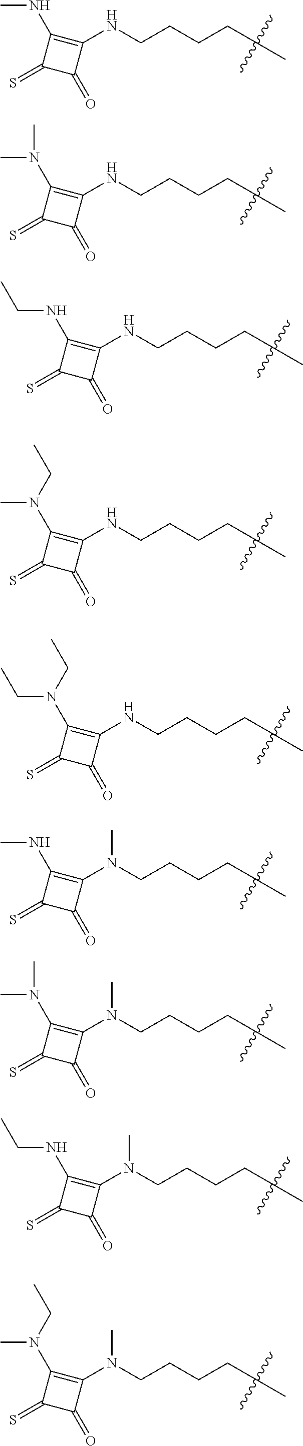

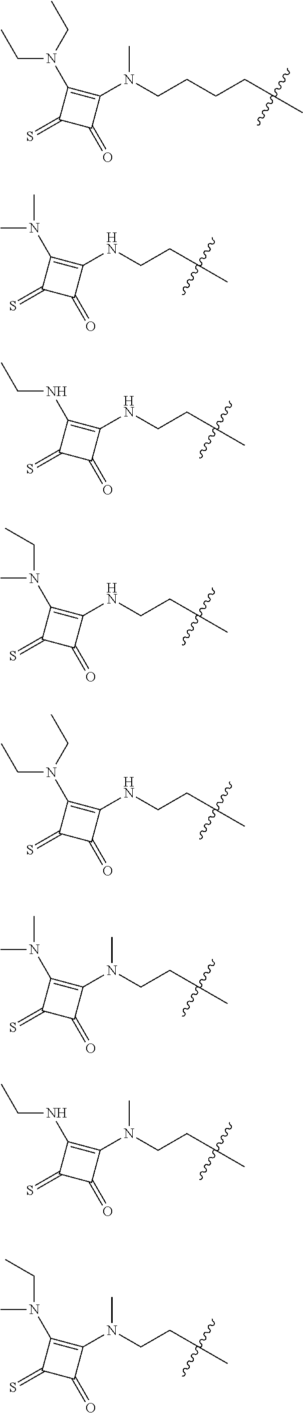

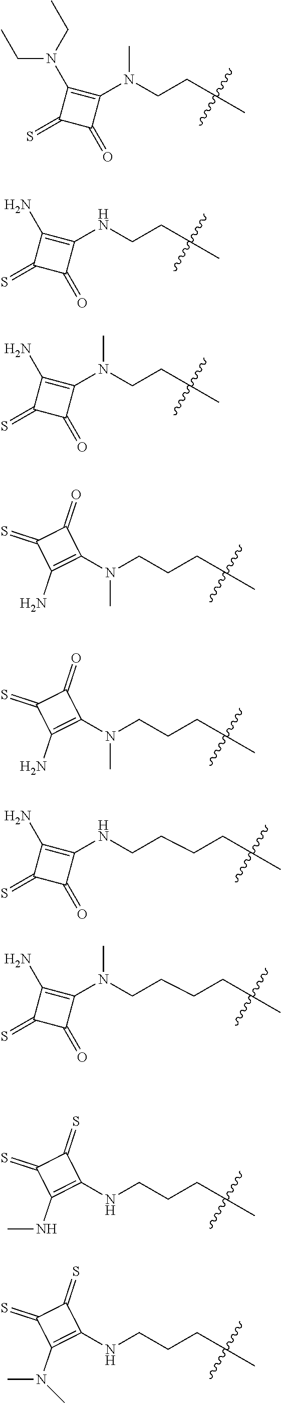

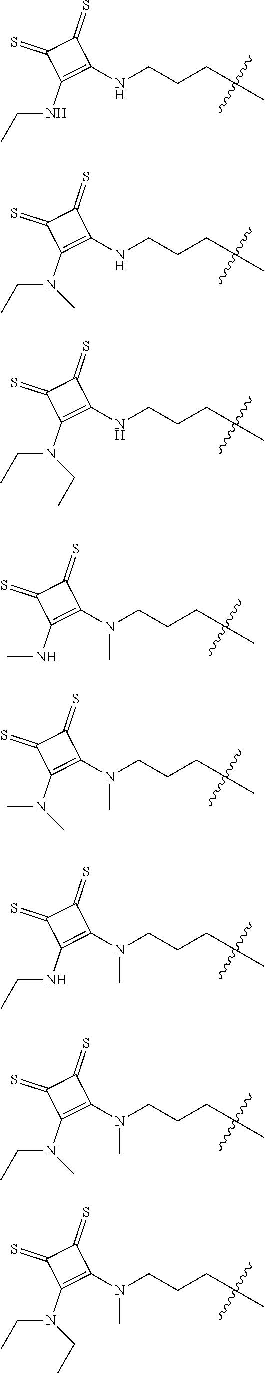

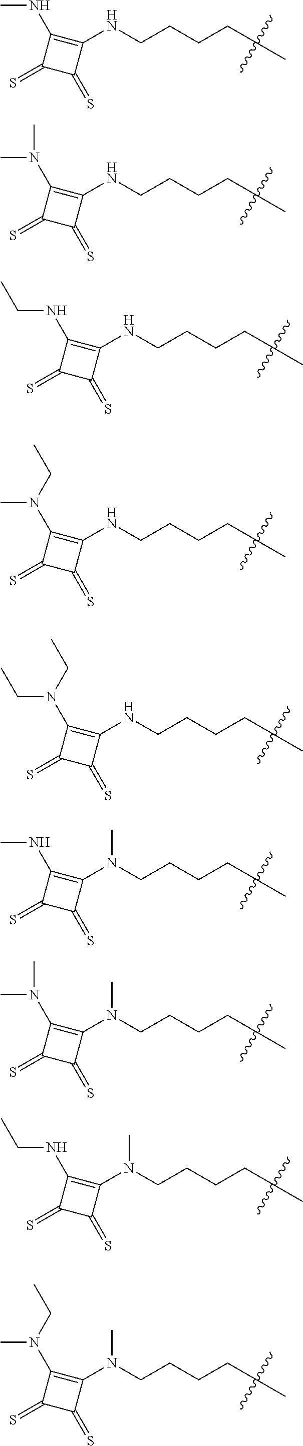

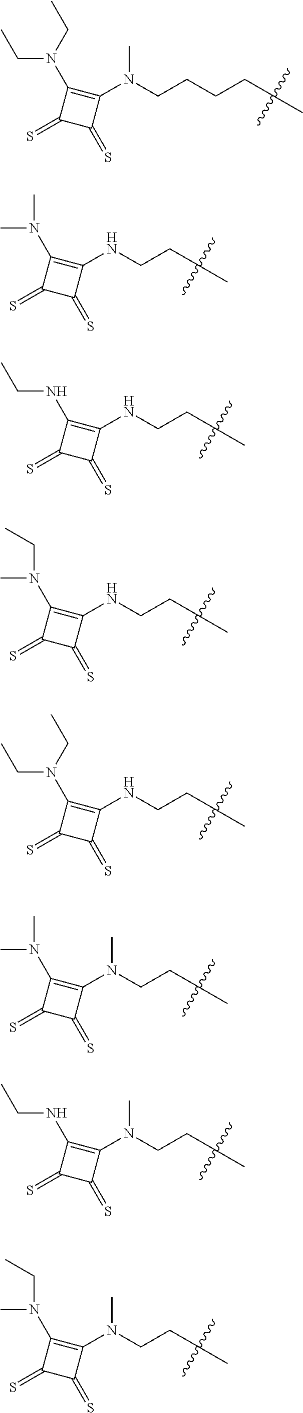

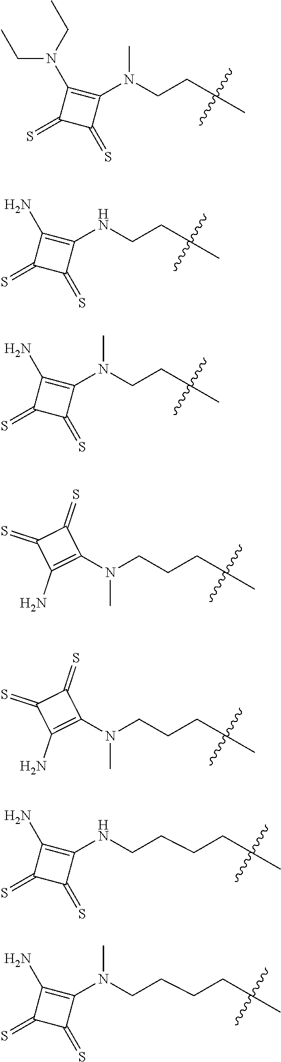

In some embodiments, a head, tail, or structure of an ionizable lipid is described in US patent application US20170210697A1.

In some embodiments, a compound has a structure according to formula 1:

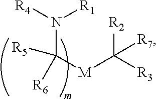

wherein:
R¹ is selected from the group consisting of C5-30 alkyl, C5-20 alkenyl, —R*YR″, YR″, and —R″M′R′;
R² and R³ are independently selected from the group consisting of H, C1-14 alkyl, C2-14 alkenyl, R*YR″, —YR″, and R*OR″, or R2 and R3, together with the atom to which they are attached, form a heterocycle or carbocycle;
R⁴ is selected from the group consisting of a C3-6 carbocycle, —(CH2)nQ, —(CH2)nCHQR, —CHQR, CO(R)2, and unsubstituted C1-6 alkyl, where Q is selected from a carbocycle, heterocycle, —OR, —O(CH2)nN(R)2, —C(O)OR, —OC(O)R, —CX3, —CX2H, —CXH2, —CN, N(R)2, —C(O)N(R)2, —N(R)C(O)R, —N(R)S(O)2R, —N(R)C(O)N(R)2, —N(R)C(S)N(R)2, —N(R)R8, —O(CH2)nOR, —N(R)C(═NR9)N(R)2, —N(R)C(═CHR9)N(R)2, OC(O)N(R)2, —N(R)C(O)OR, —N(OR)C(O)R, —N(OR)S(O)2R, —N(OR)C(O)OR, —N(OR)C(O)N(R)2, —N(OR)C(S)N(R)2, —N(OR)C(—NR)N(R), —N(OR)C(═CHR9)N(R)2, —C(═NR9)N(R)2, —C(═NR9)R, —C(O)N(R)OR, and —C(R)N(R)2, C(O)OR, and each n is independently selected from 1, 2, 3, 4, and 5 or a head group disclosed in Table 1;
each R⁵ is independently selected from the group consisting of C1-3 alkyl, C2-3 alkenyl, and H;
each R⁶ is independently selected from the group consisting of C1-3 alkyl, C2-3 alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)—, —S—S—, an aryl group, and a heteroaryl group;
R⁷ is selected from the group consisting of C1-3alkyl, C2-3 alkenyl, and H;
R8 is selected from the group consisting of C3-6 carbocycle and heterocycle;
R9 is selected from the group consisting of H, CN, NO2, C1-6 alkyl, —OR, S(O)2R, —S(O)2N(R)2, C2-6 alkenyl, C3-6 carbocycle and heterocycle;
each R is independently selected from the group consisting of C1-3 alkyl, C2-3 alkenyl, and H;
each R′ is independently selected from the group consisting of C1-18 alkyl, C2-18 alkenyl, R*YR″, —YR″, and H;
each R″ is independently selected from the group consisting of C3-14 alkyl, C3-14 alkenyl, and H;
each R* is independently selected from the group consisting of C1-12 alkyl and C2-12 alkenyl:
each Y is independently a C3-6 carbocycle;
each X is independently selected from the group consisting of F, Cl, Br, and I;
each Q is is —OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; and
m is selected from 5, 6, 7, 8, 9, 10, 11, 12, and 13:
and wherein when R⁴ is —(CH2)nQ, —(CH2)nCHQR, —CHQR, or —CQ(R)2, then (i) Q is not —N(R), when n is 1, 2, 3, 4 or 5, or (ii) Q is not 5, 6, or 7-membered heterocycloalkyl when n is 1 or 2.

In some embodiments, R4 is in Table 1. In some embodiments, R4 in formula 1 is selected from head groups 1-47.

In some embodiments, a subset of the compounds of formula 1 are also described by formula 1b:

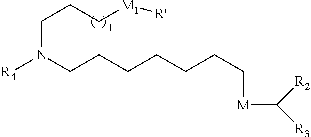

Wherein 1 is selected from 1, 2, 3, 4, and 5; M1 is a bond or M′; R⁴ is unsubstituted C1-3 alkyl, or —(CH2)nQ, in which n is 2, 3, or 4, and Q is —OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl.

In some embodiments, a head, tail, or structure of an ionizable lipid is described in international patent application PCT/US2018/058555.

In some embodiments, an ionizable lipid has a structure according to formula 2:

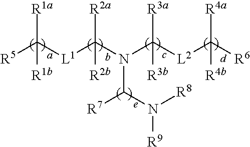

wherein:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC(═O)O— or a direct bond;
Ra is H or C1-C12 alkyl;
R1a and R1b are, at each occurrence, independently either (a) H or C1-C12 alkyl, or (b) R1a is H or C1-C12 alkyl, and R1b together with the carbon atom to which it is bound is taken together with an adjacent R1b and the carbon atom to which it is bound to form a carbon-carbon double bond;
R2a and R2b are, at each occurrence, independently either (a) H or C1-C12 alkyl, or (b) R2a is H or C1-C12 alkyl, and R2b together with the carbon atom to which it is bound is taken together with an adjacent R2b and the carbon atom to which it is bound to form a carbon-carbon double bond;
R3a and R3b are, at each occurrence, independently either (a) H or C1-C12 alkyl, or (b) R3a is H or C1-C12 alkyl, and R3b together with the carbon atom to which it is bound is taken together with an adjacent R3b and the carbon atom to which it is bound to form a carbon-carbon double bond;
R4a and R4b are, at each occurrence, independently either (a) H or C1-C12 alkyl, or (b) R4a is H or C1-C12 alkyl, and R4b together with the carbon atom to which it is bound is taken together with an adjacent R4b and the carbon atom to which it is bound to form a carbon-carbon double bond;
R5 and R6 are each independently methyl or cycloalkyl;
R7 is, at each occurrence, independently H or C1-C12 alkyl;
R8 and R9 are each independently unsubstituted C1-C12 alkyl; or R8 and R9, together with the nitrogen atom to which they are attached, form a 5, 6 or 7-membered heterocyclic ring comprising one nitrogen atom;
a and d are each independently an integer from 0 to 24;
b and c are each independently an integer from 1 to 24;
e is 1 or 2; and
x is 0, 1 or 2.

In some embodiments, an ionizable lipid has a structure according to formula 3:

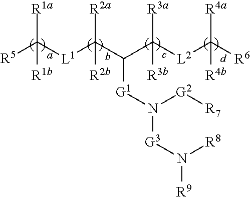

wherein:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC(═O)O— or a direct bond;
G1 is C1-C2 alkylene, —(C═O)—, —O(C═O)—, —SC(═O)—, —NRaC(═O)— or a direct bond:
G2 is —C(═O)—, —(C═O)O—, —C(═O)S—, —C(═O)NRa— or a direct bond;
G3 is C₁-C₆ alkylene;
Ra is H or C₁-C₁₂ alkyl;
R1a and R1b are, at each occurrence, independently either: (a) H or C1-C12 alkyl; or (b) R1a is H or C1-C12 alkyl, and R1b together with the carbon atom to which it is bound is taken together with an adjacent R1b and the carbon atom to which it is bound to form a carbon-carbon double bond;
R2a and R2b are, at each occurrence, independently either: (a) H or C1-C12 alkyl; or (b) R2a is H or C1-C12 alkyl, and R2b together with the carbon atom to which it is bound is taken together with an adjacent R2b and the carbon atom to which it is bound to form a carbon-carbon double bond;
R3a and R3b are, at each occurrence, independently either (a): H or C1-C12 alkyl; or (b) R3a is H or C1-C12 alkyl, and R3b together with the carbon atom to which it is bound is taken together with an adjacent R3b and the carbon atom to which it is bound to form a carbon-carbon double bond;
R4a and R4b are, at each occurrence, independently either: (a) H or C1-C12 alkyl; or (b) R4a is H or C1-C12 alkyl, and R4b together with the carbon atom to which it is bound is taken together with an adjacent R4b and the carbon atom to which it is bound to form a carbon-carbon double bond;
R5 and R6 are each independently H or methyl;
R7 is C4-C20 alkyl;
R8 and R9 are each independently C1-C12 alkyl; or R8 and R9, together with the nitrogen atom to which they are attached, form a 5, 6 or 7-membered heterocyclic ring;
a, b, c and d are each independently an integer from 1 to 24; and x is 0, 1 or 2.

In some embodiments, an ionizable lipid has a structure according to formula 4:

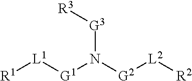

wherein:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa— or —NRaC(═O)O— or a direct bond;
G1 and G2 are each independently unsubstituted C1-C12 alkylene or C1-C12 alkenylene;
G3 is C1-C24 alkylene, C1-C24 alkenylene, C3-C8 cycloalkylene, C3-C8 cycloalkenylene;
Ra is H or C1-C12 alkyl;
R1 and R2 are each independently C6-C24 alkyl or C6-C24 alkenyl;
R3 is H, OR5, CN, —C(═O)OR4, —OC(═O)R4 or —NR5C(═O)R4;
R4 is C1-C12 alkyl;
R5 is H or C1-C6 alkyl; and
x is 0, 1 or 2.

In some embodiments, an ionizable lipid has a structure according to formula 5:

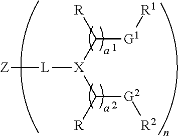

wherein:
one of G1 or G2 is, at each occurrence, —O(C═O)—, —(C═O)O—, —C(═O)—, —O—, —S(O)y, —S—S—, —C(═O)S—, SC(═O)—, —N(Ra)C(═O)—, —C(═O)N(Ra)—, —N(Ra)C(═O)N(Ra)—, —OC(═O)N(Ra)— or —N(Ra)C(═O)O—, and the other of G1 or G2 is, at each occurrence, —O(C═O)—, —(C═O)O—, —C(═O)—, —O—, —S(O)y, —S—S—, —C(═O)S—, —SC(═O)—, —N(Ra)C(═O)—, —C(═O)N(Ra)—, —N(Ra)C(═O)N(Ra)—, —OC(═O)N(Ra)— or —N(Ra)C(═O)O— or a direct bond;
L is, at each occurrence, —O(C═O)—, wherein — represents a covalent bond to X;
X is CRa;
Z is alkyl, cycloalkyl or a monovalent moiety comprising at least one polar functional group when n is 1; or Z is alkylene, cycloalkylene or a polyvalent moiety comprising at least one polar functional group when n is greater than 1;
Ra is, at each occurrence, independently H, C1-C12 alkyl, C1-C12 hydroxylalkyl, C1-C12 aminoalkyl, C1-C12 alkylaminylalkyl, C1-C12 alkoxyalkyl, C1-C12 alkoxycarbonyl, C1-C12 alkylcarbonyloxy, C1-C12 alkylcarbonyloxyalkyl or C1-C12 alkylcarbonyl;
R is, at each occurrence, independently either: (a) H or C1-C12 alkyl; or (b) R together with the carbon atom to which it is bound is taken together with an adjacent R and the carbon atom to which it is bound to form a carbon-carbon double bond;
R1 and R2 have, at each occurrence, the following structure, respectively:

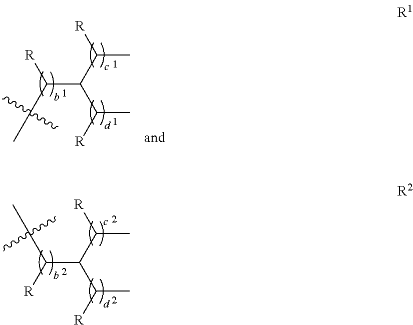

a1 and a2 are, at each occurrence, independently an integer from 3 to 12;
b1 and b2 are, at each occurrence, independently 0 or 1;
c1 and c2 are, at each occurrence, independently an integer from 5 to 10;
d1 and d2 are, at each occurrence, independently an integer from 5 to 10;
y is, at each occurrence, independently an integer from 0 to 2; and
n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, an ionizable lipid has a structure according to formula 6:

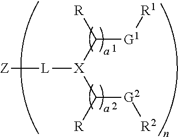

wherein:
one of G1 or G2 is, at each occurrence, —O(C═O)—, —(C═O)O—, —C(═O)—, —O—, —S(O)y, —S—S—, —C(═O)S—, SC(═O)—, —N(Ra)C(═O)—, —C(═O)N(Ra)—, —N(Ra)C(═O)N(Ra)—, —OC(═O)N(Ra)— or —N(Ra)C(═O)O—, and the other of G1 or G2 is, at each occurrence, —O(C═O)—, —(C═O)O—, —C(═O)—, —O—, —S(O)y-, —S—S—, —C(═O)S—, —SC(═O)—, —N(Ra)C(═O)—, —C(═O)N(Ra)—, —N(Ra)C(═O)N(Ra)—, —OC(═O)N(Ra)— or —N(Ra)C(═O)O— or a direct bond;
L is, at each occurrence, —O(C═O)—, wherein — represents a covalent bond to X;
X is CRa;
Z is alkyl, cycloalkyl or a monovalent moiety comprising at least one polar functional group when n is 1; or Z is alkylene, cycloalkylene or a polyvalent moiety comprising at least one polar functional group when n is greater than 1;
Ra is, at each occurrence, independently H, C1-C12 alkyl, C1-C12 hydroxylalkyl, C1-C12 aminoalkyl, C1-C12 alkylaminylalkyl, C1-C12 alkoxyalkyl, C1-C12 alkoxycarbonyl, C1-C12 alkylcarbonyloxy, C1-C12 alkylcarbonyloxyalkyl or C1-C12 alkylcarbonyl;
R is, at each occurrence, independently either: (a) H or C1-C12 alkyl; or (b) R together with the carbon atom to which it is bound is taken together with an adjacent R and the carbon atom to which it is bound to form a carbon-carbon double bond;
R1 and R2 have, at each occurrence, the following structure, respectively:

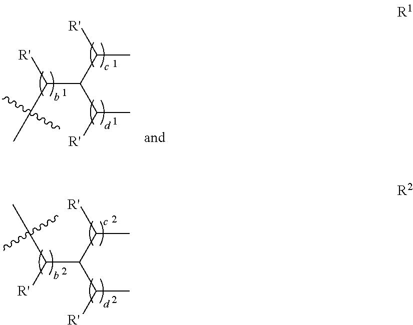

R′ is, at each occurrence, independently H or C1-C12 alkyl;
a1 and a2 are, at each occurrence, independently an integer from 3 to 12;
b1 and b2 are, at each occurrence, independently 0 or 1;
c1 and c2 are, at each occurrence, independently an integer from 2 to 12;
d1 and d2 are, at each occurrence, independently an integer from 2 to 12;
y is, at each occurrence, independently an integer from 0 to 2; and
n is an integer from 1 to 6,
wherein a1, a2, c1, c1, d1 and d1 are selected such that the sum of a1+c1+d1 is an integer from 18 to 30, and the sum of a2+c2+d2 is an integer from 18 to 30, and wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In certain embodiments of Formula (V), G1 and G2 are each independently —O(C═O)— or —(C═O)O—.

In some embodiments, an ionizable lipid is selected from the following, and/or has the head or one or more tails of one of the following:

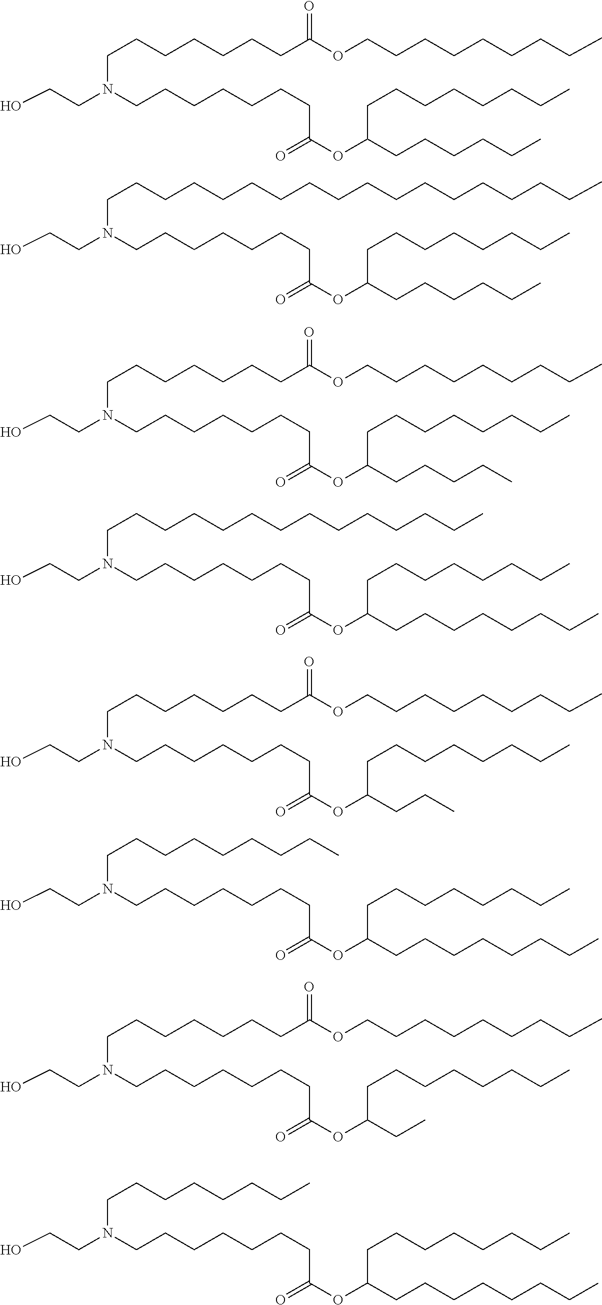

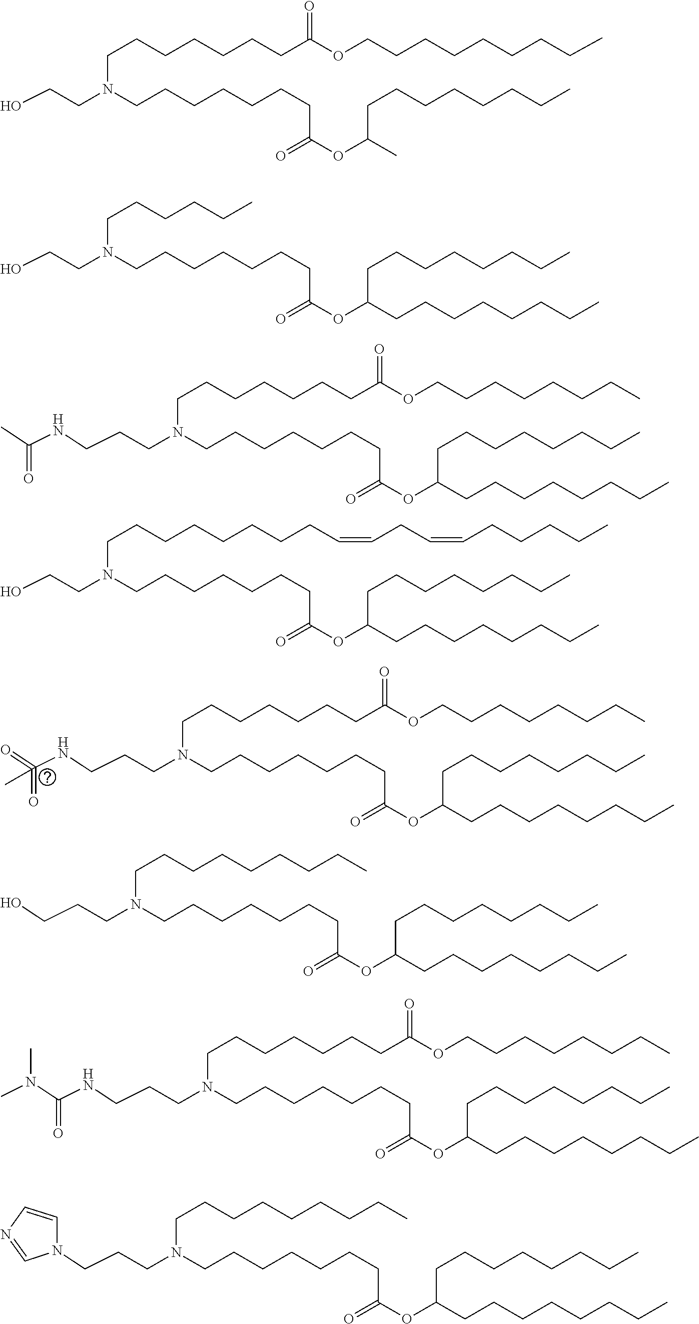

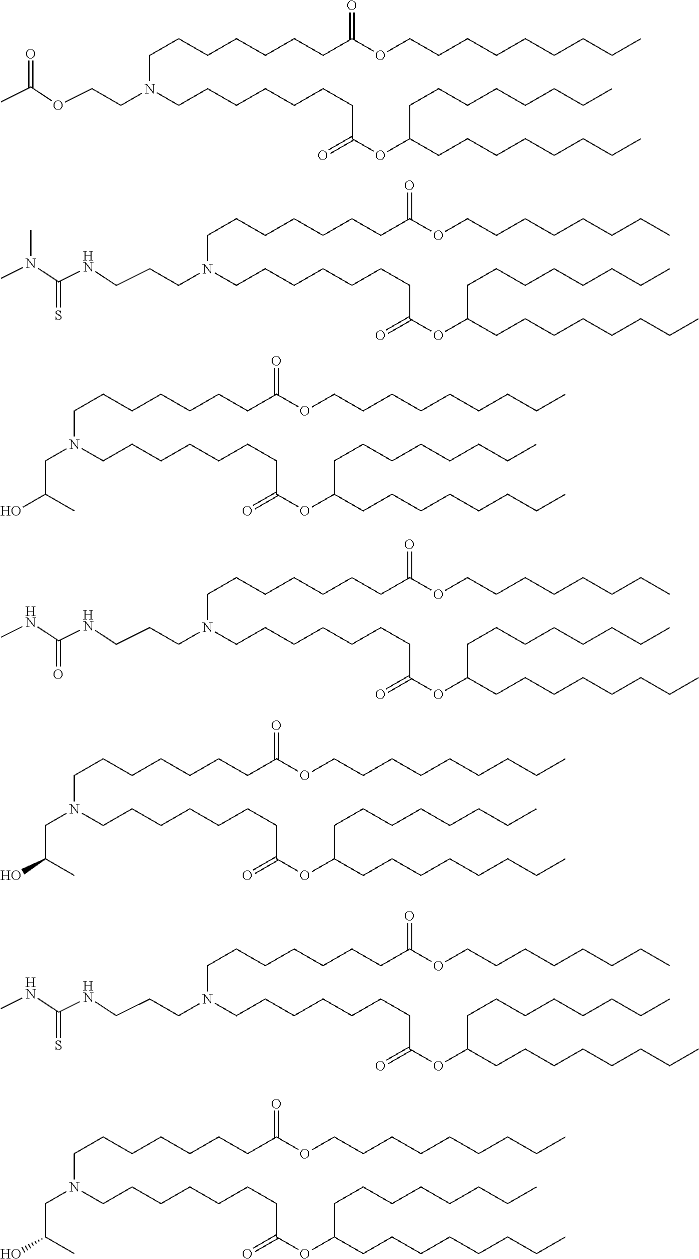

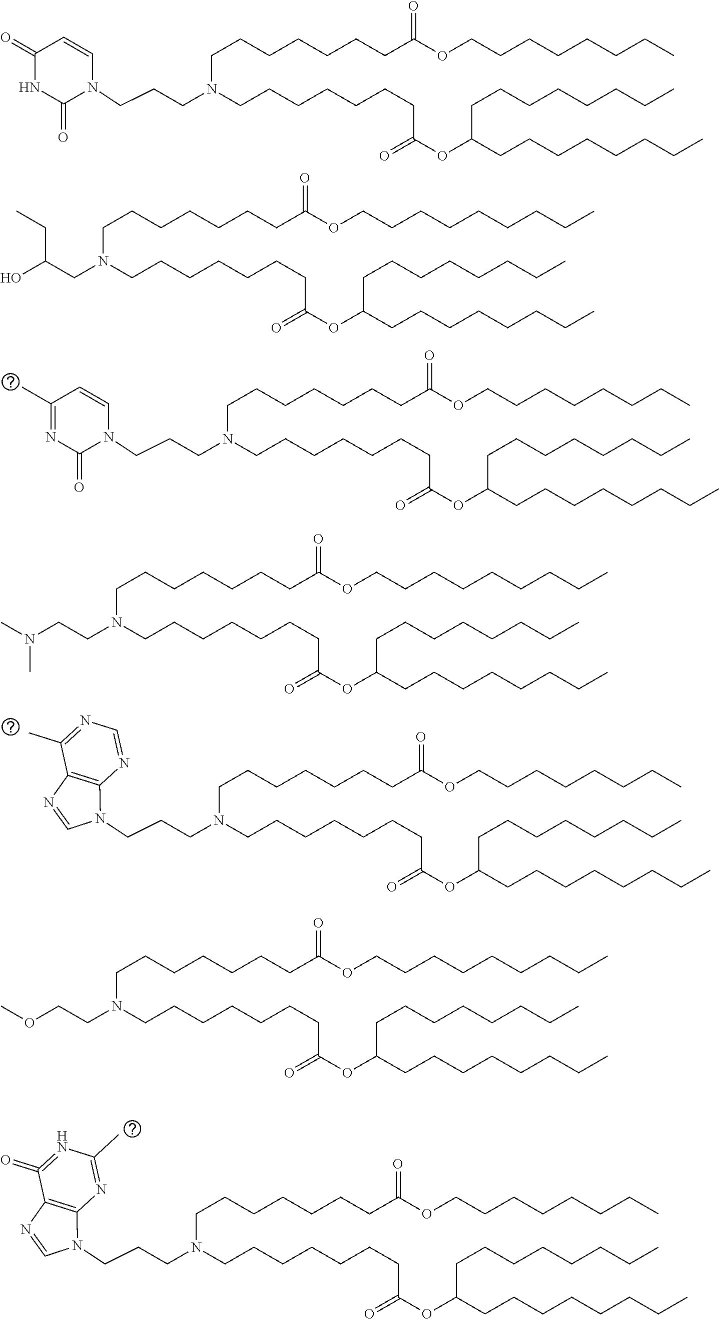

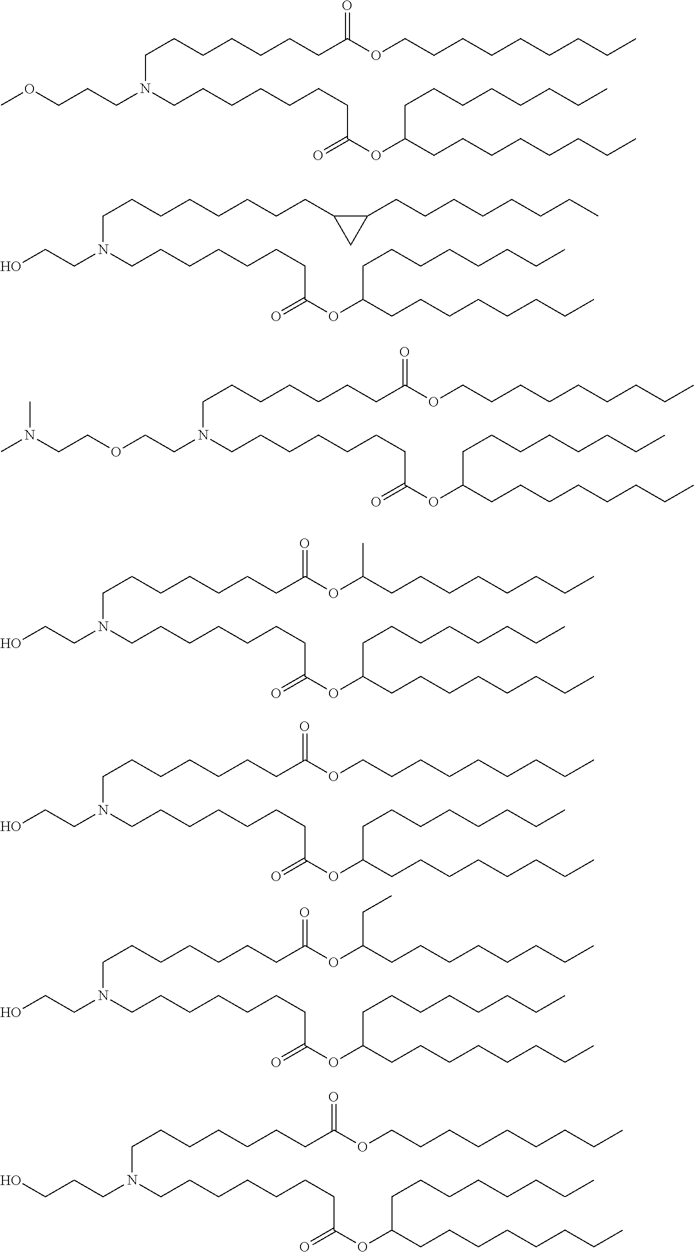

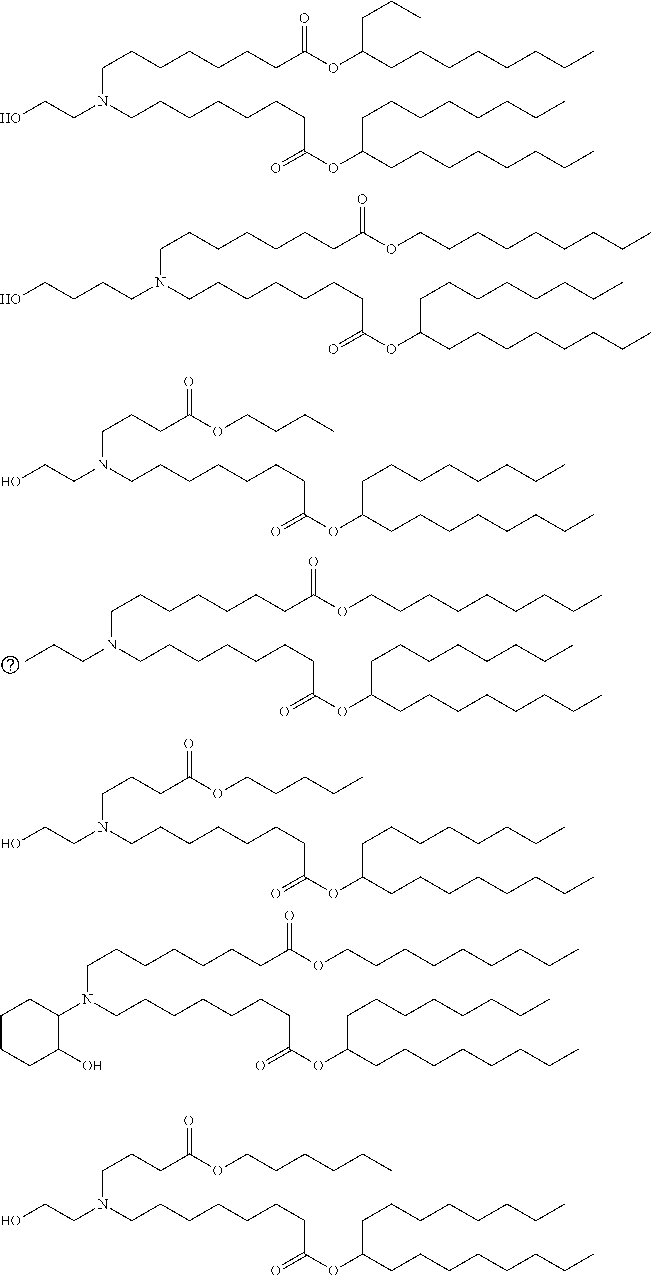

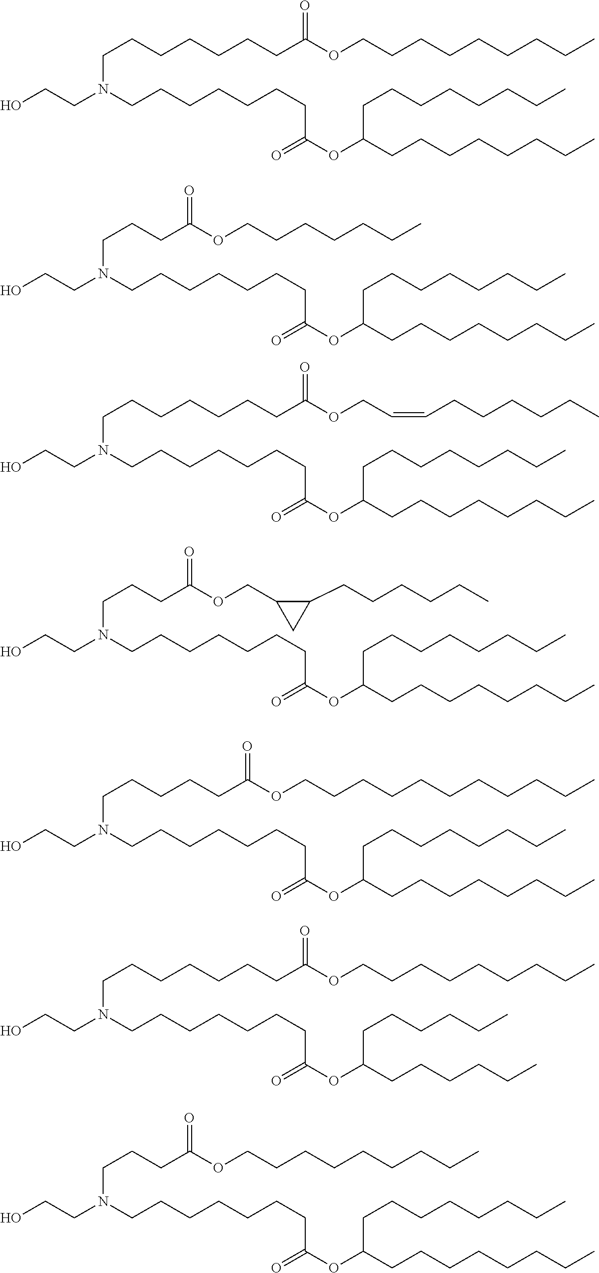

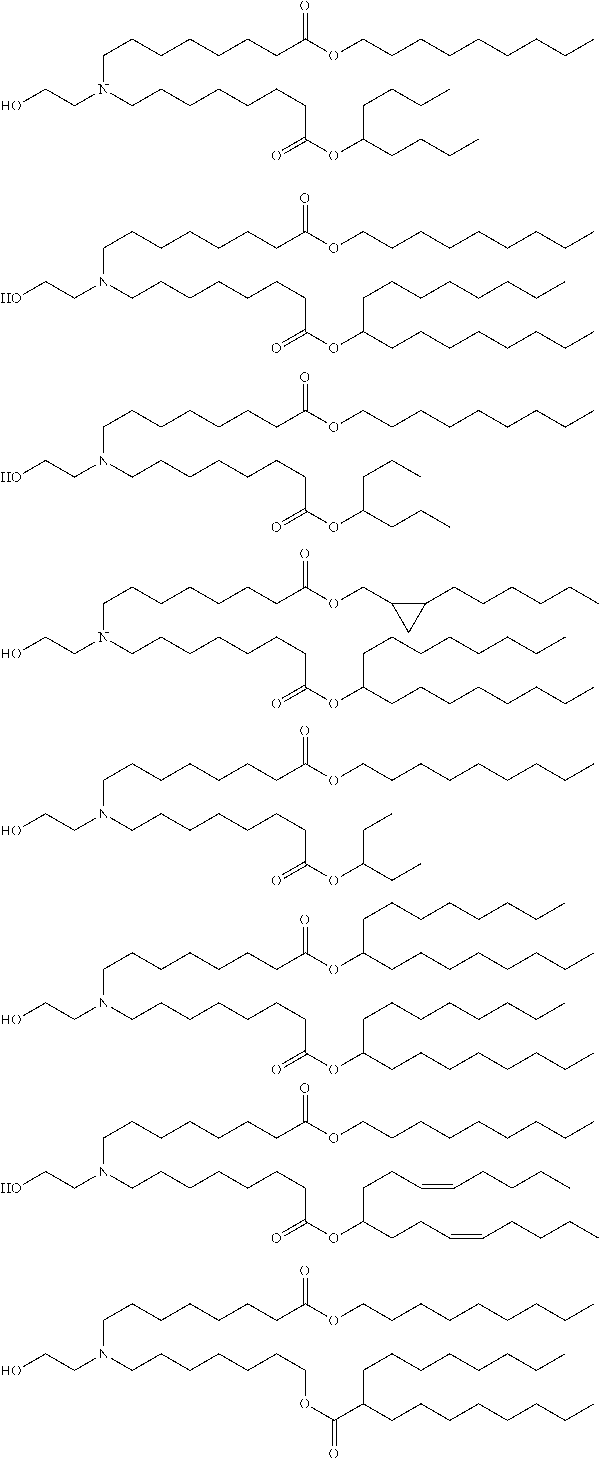

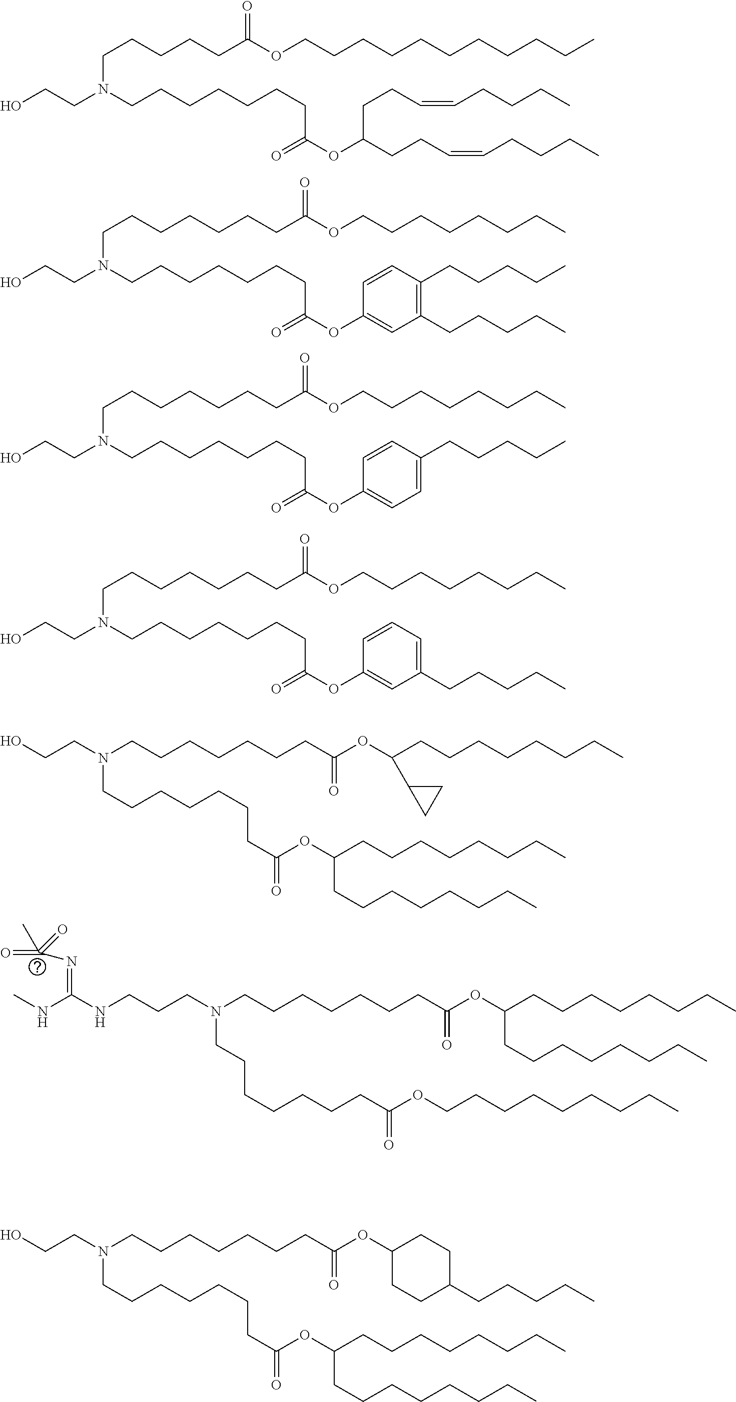

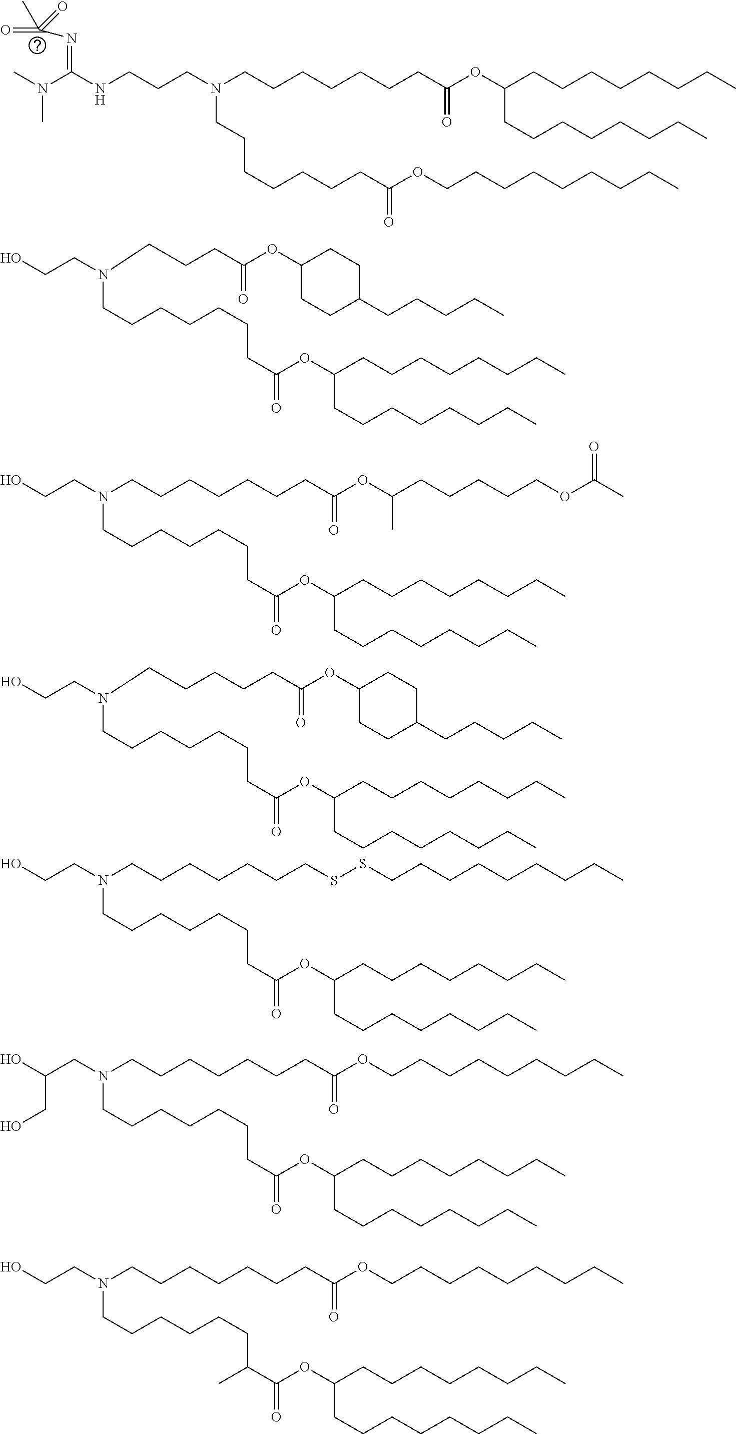

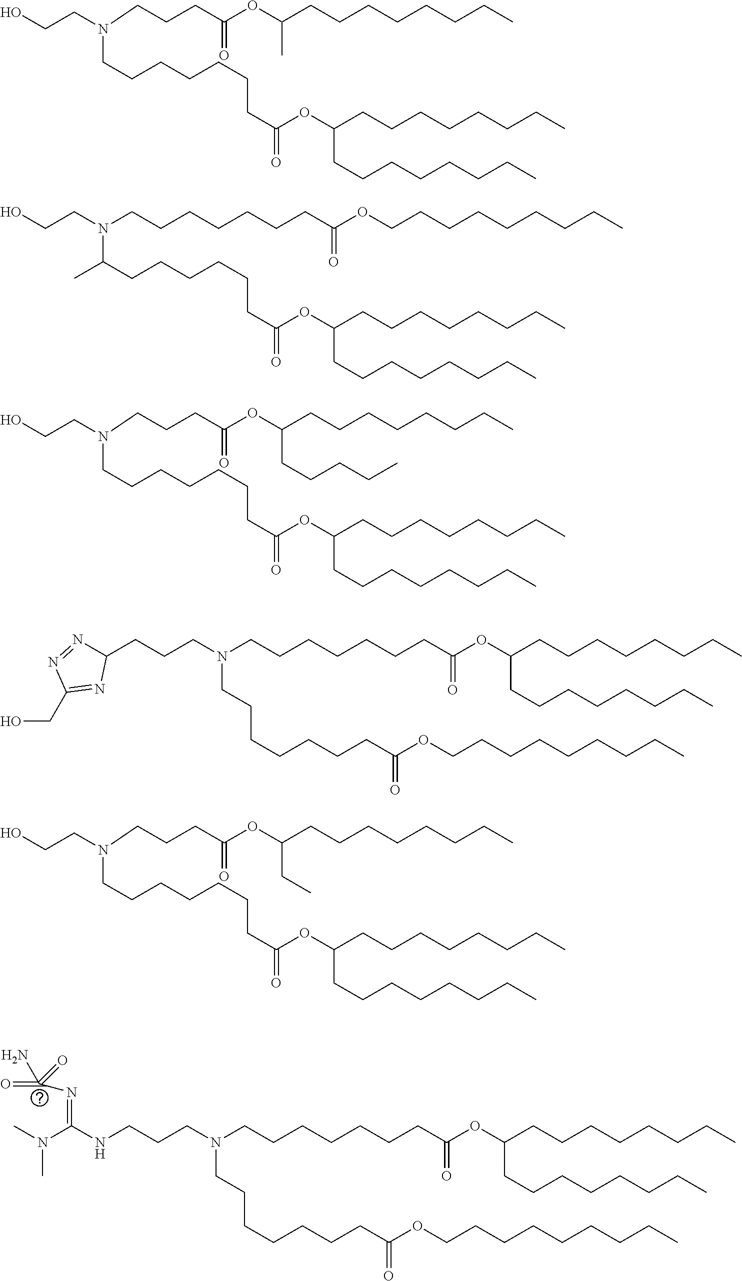

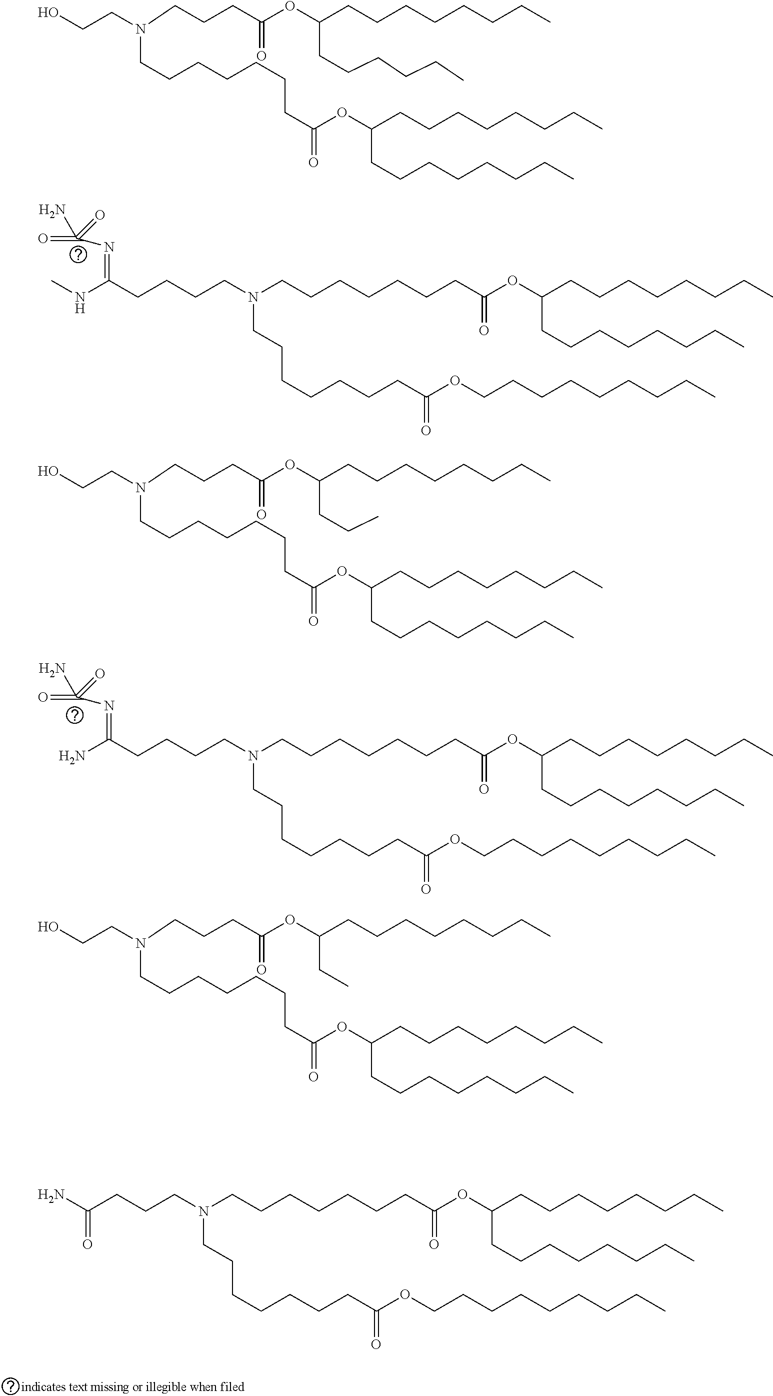

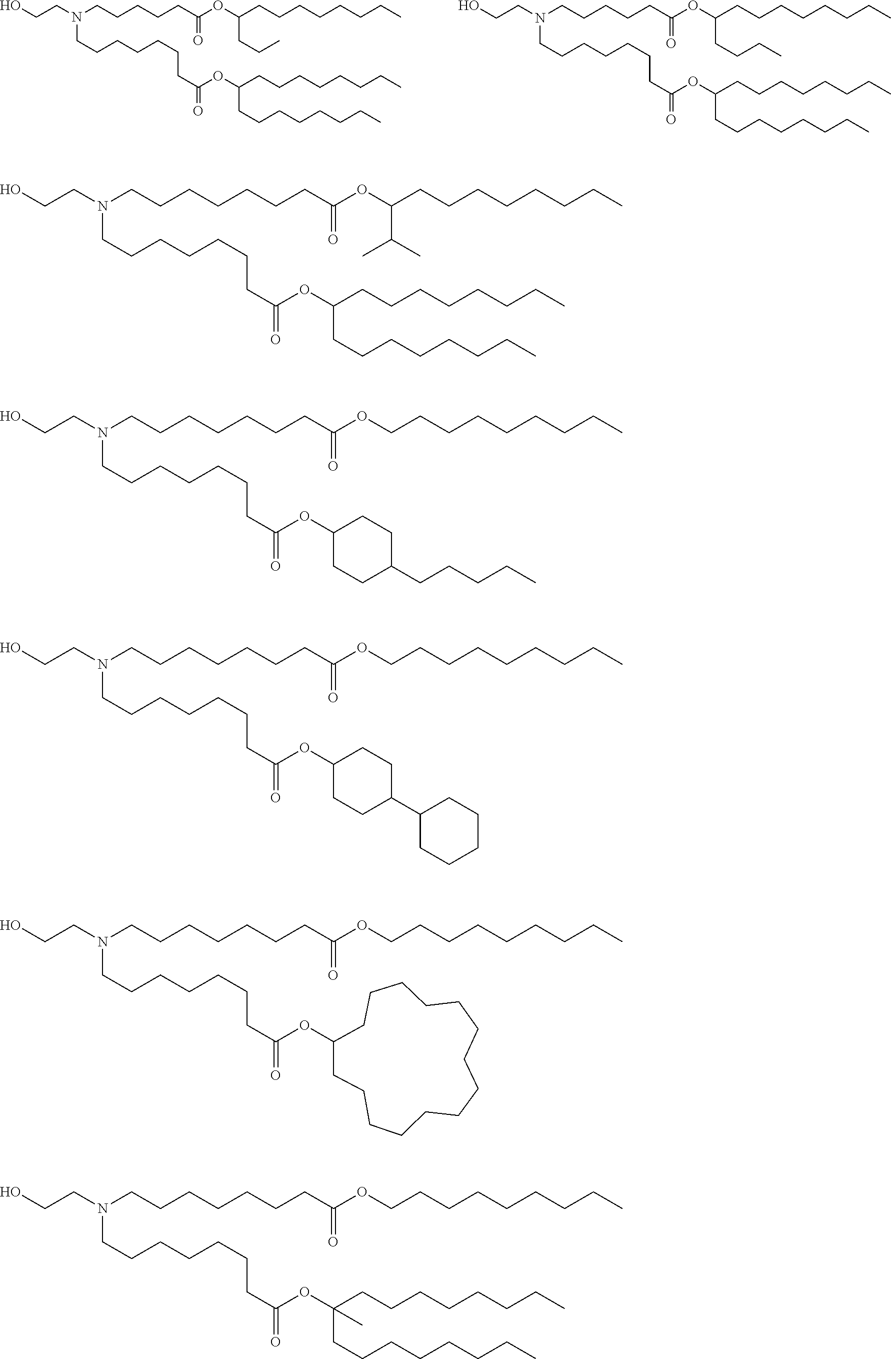

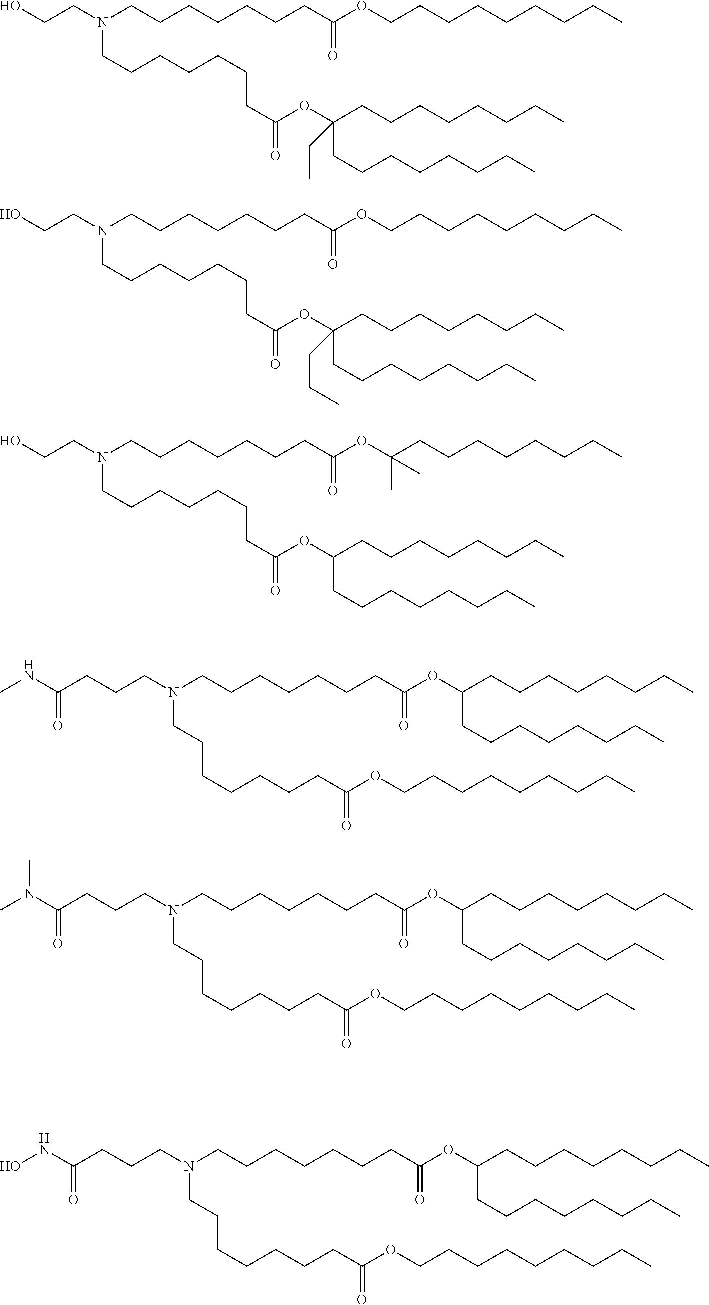

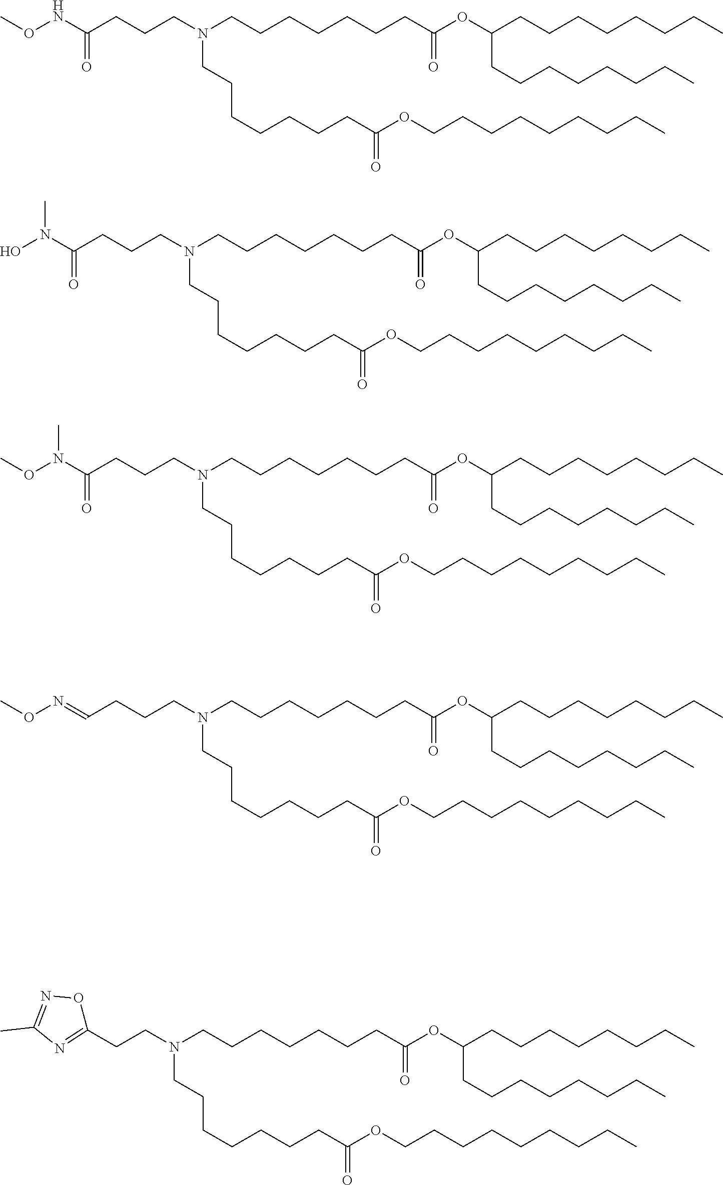

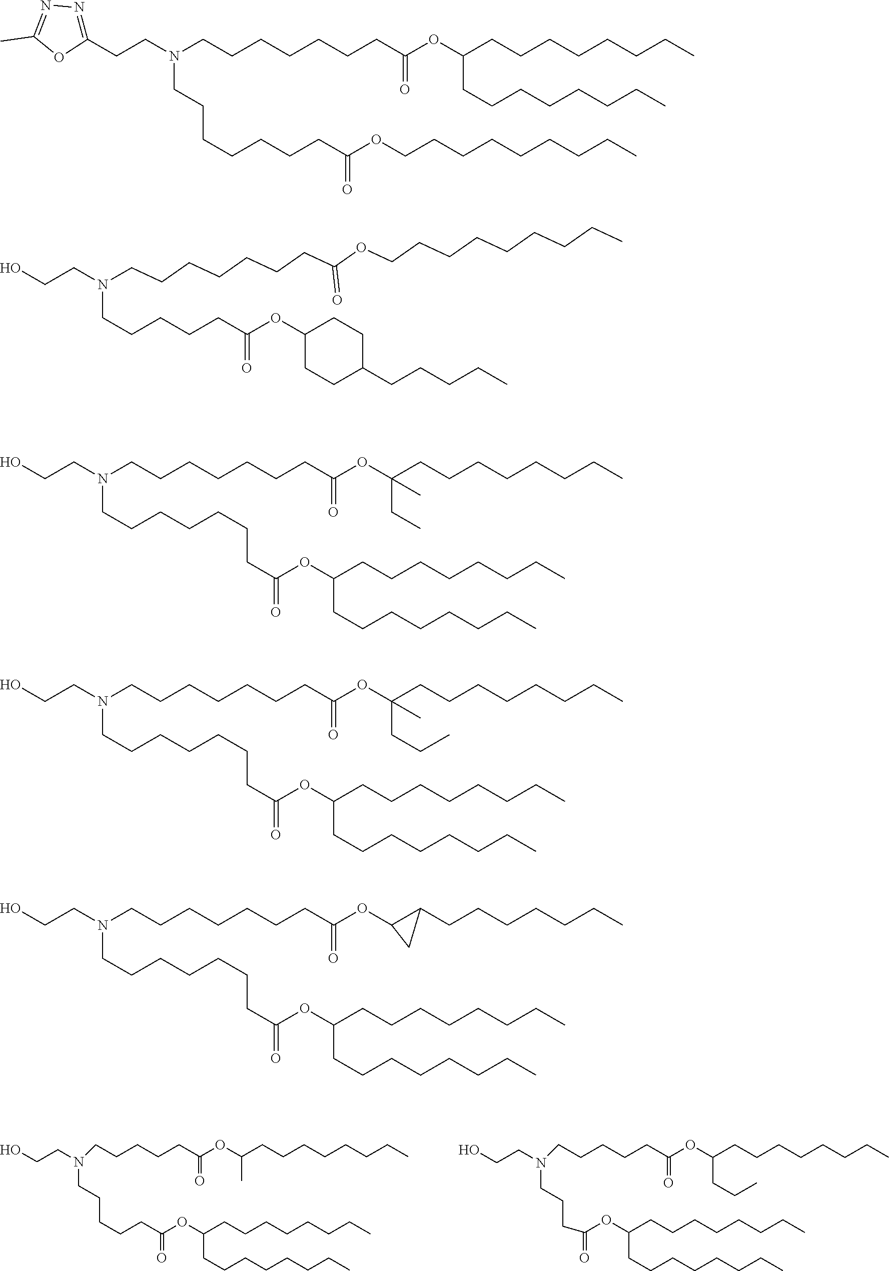

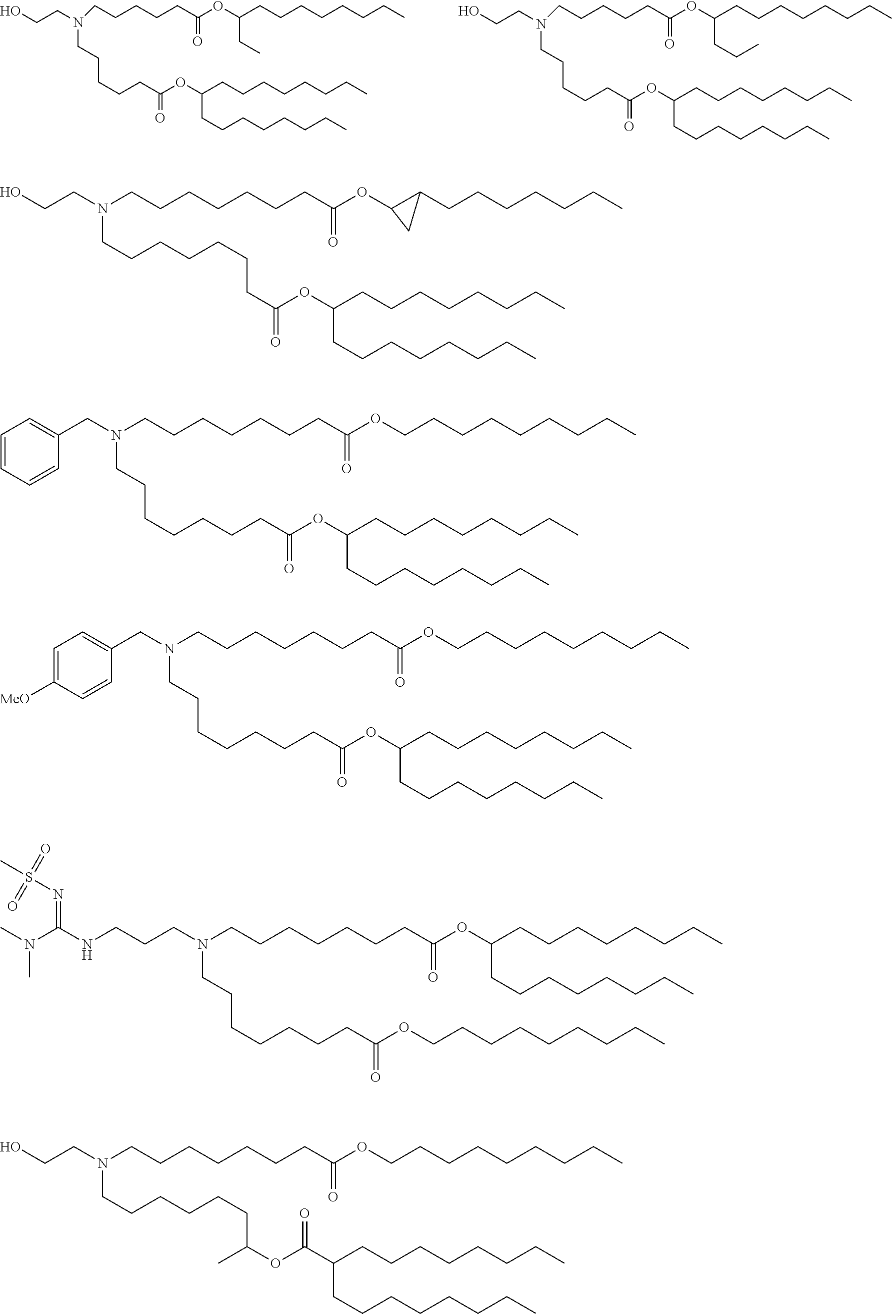

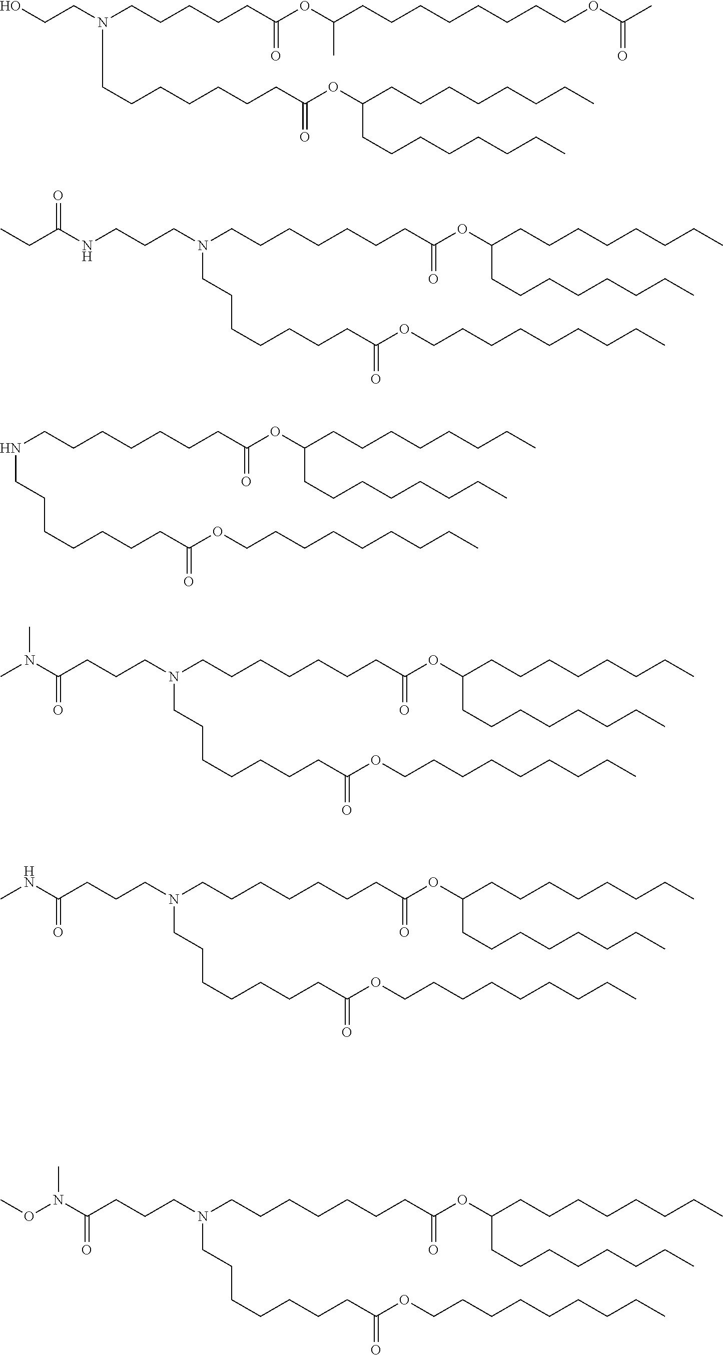

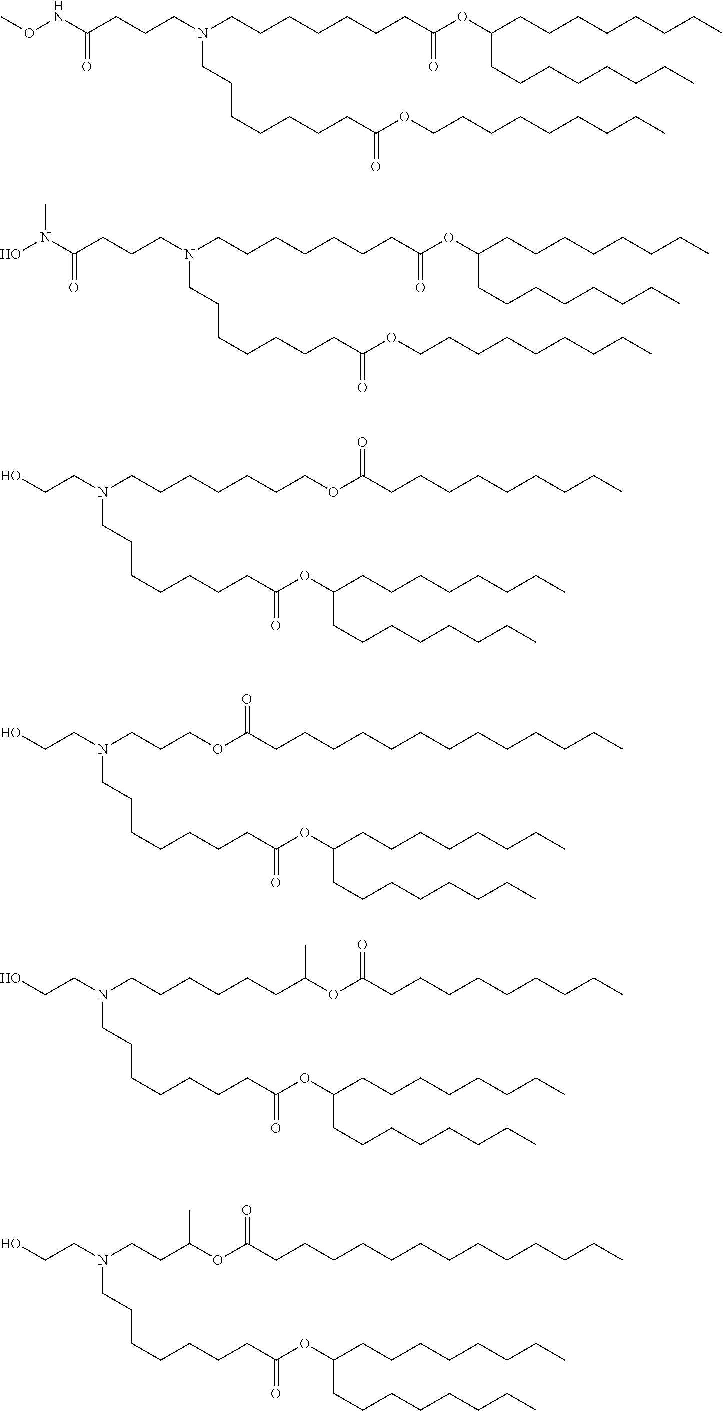

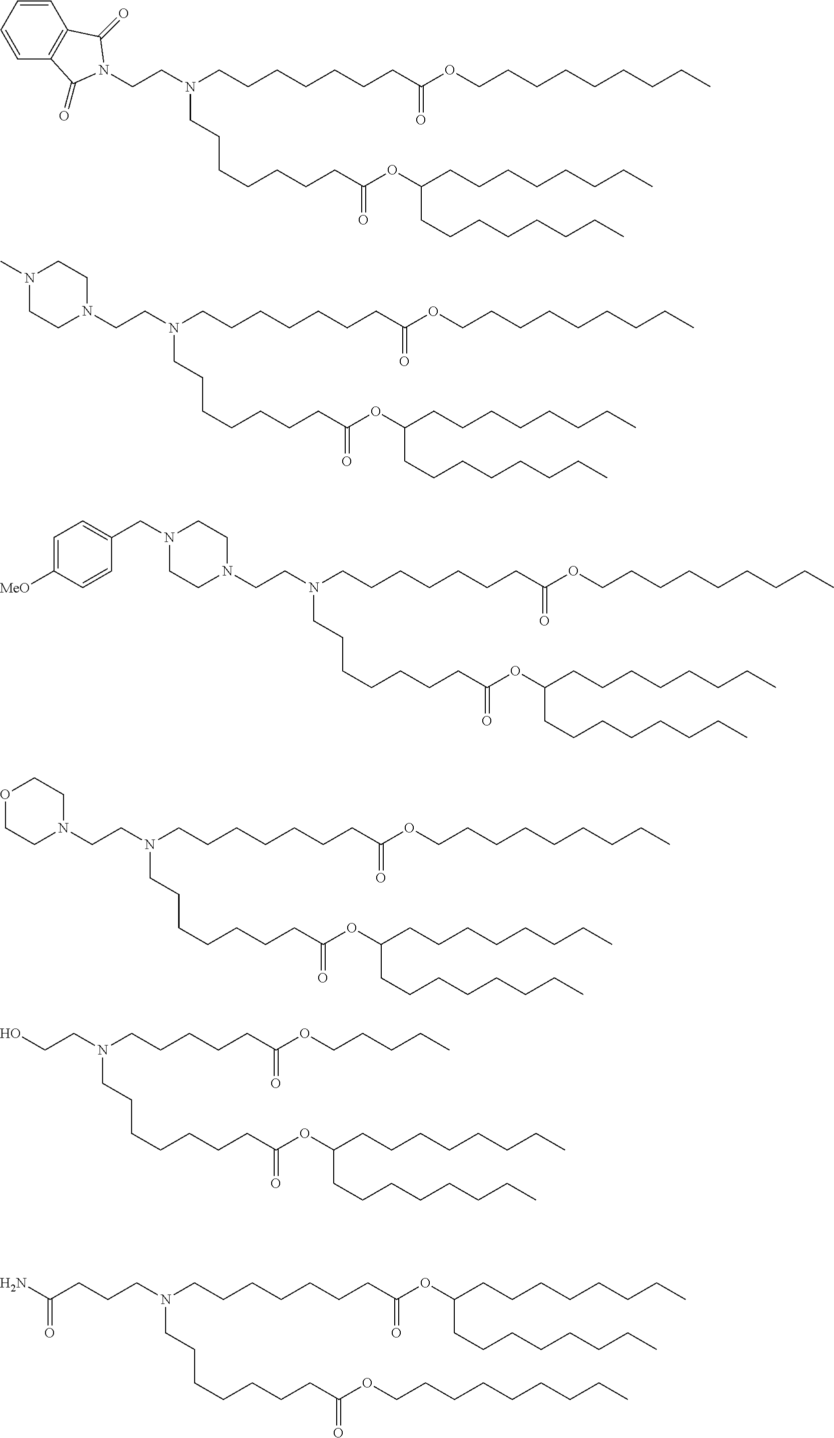

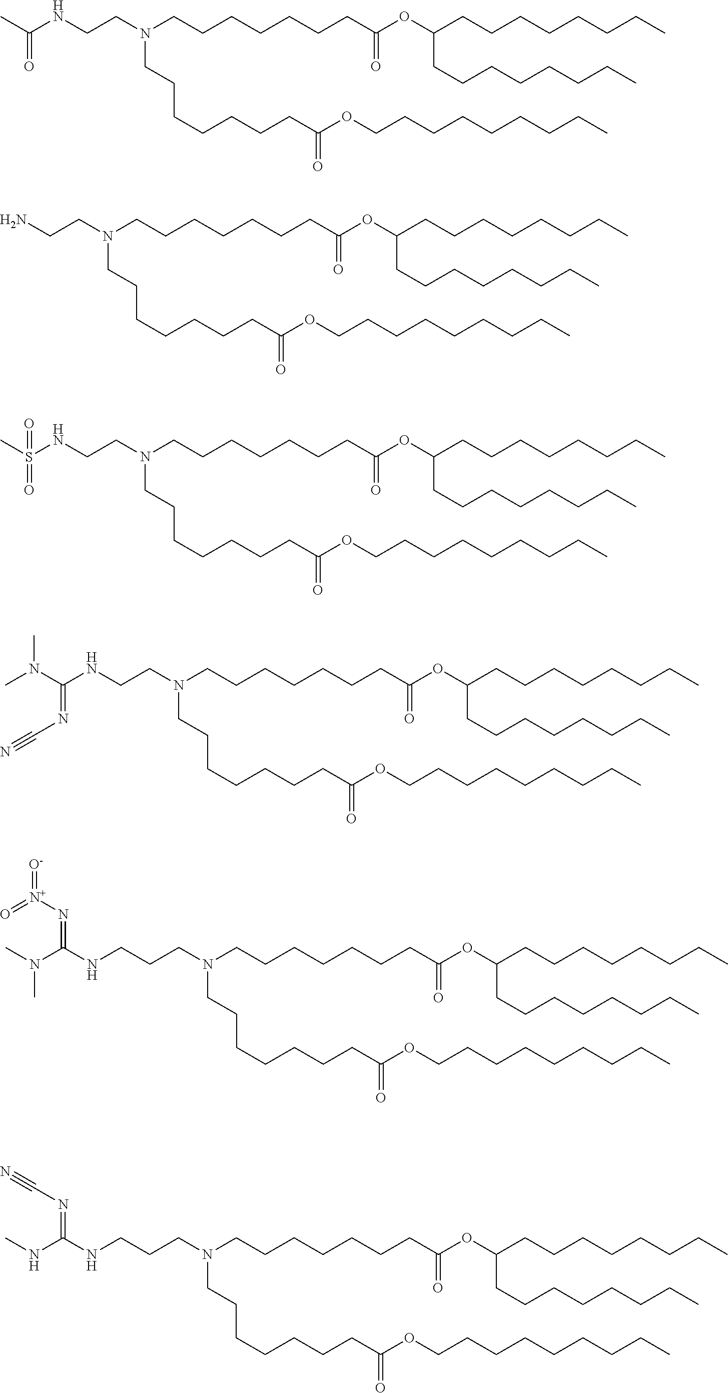

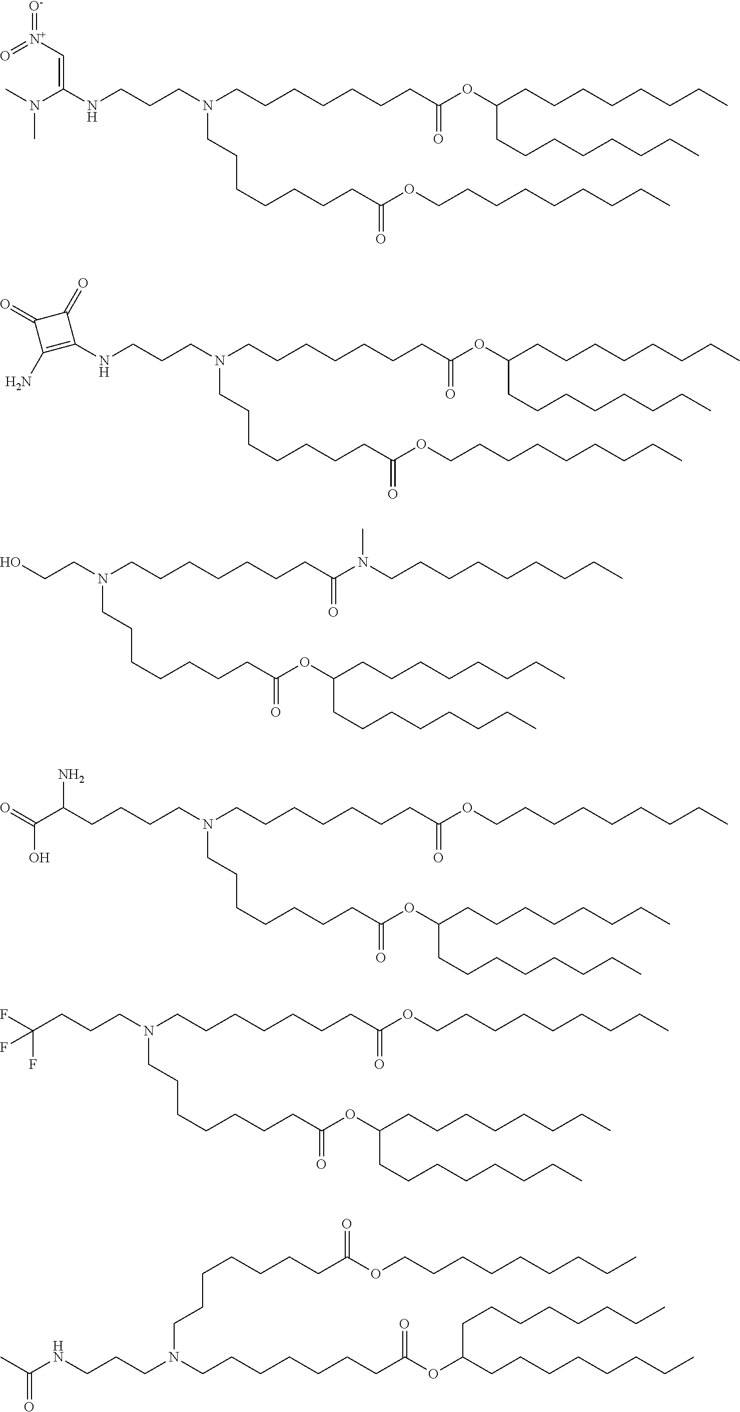

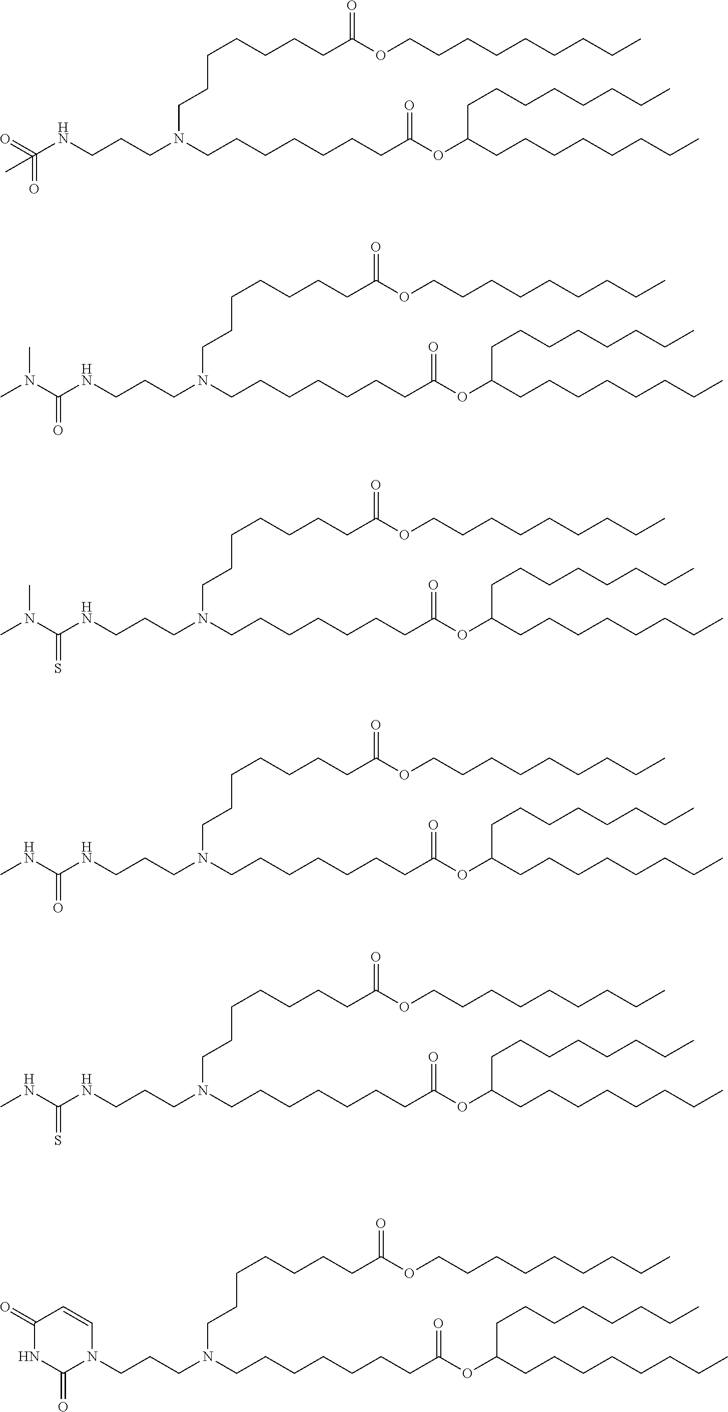

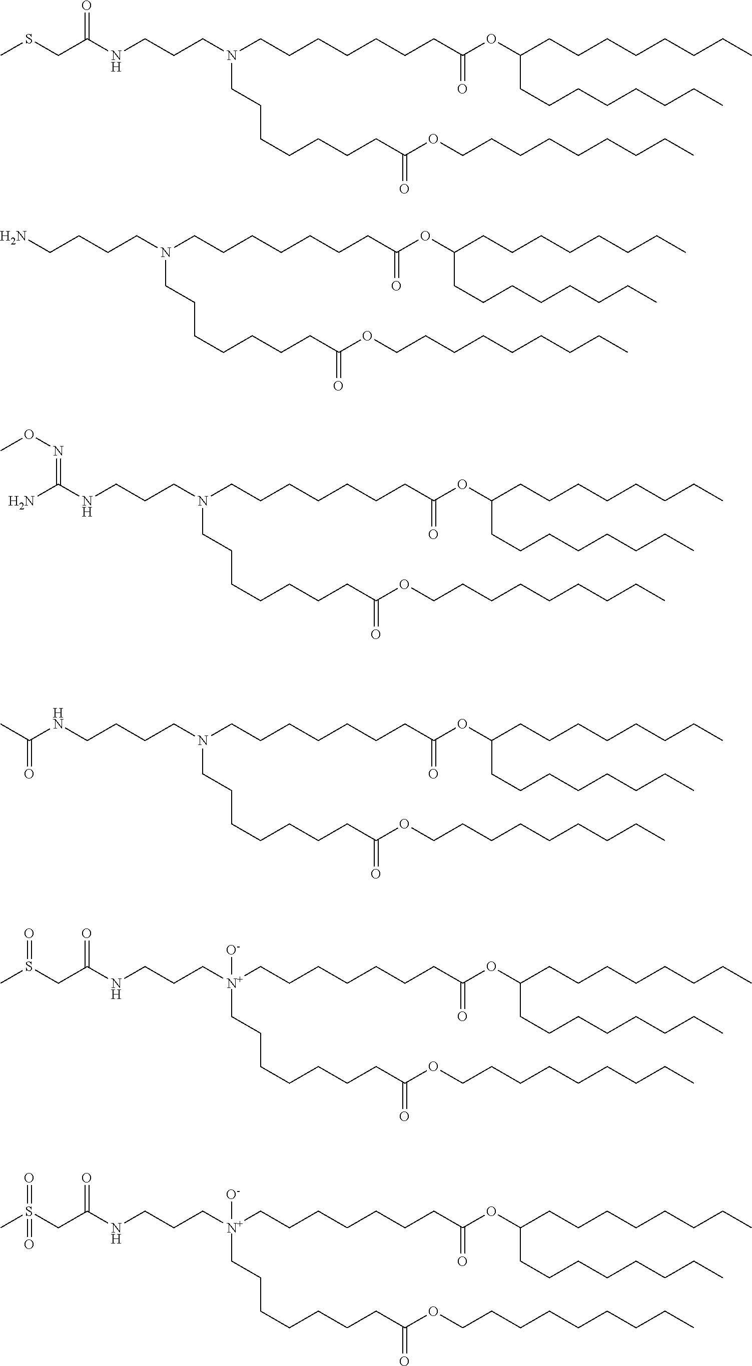

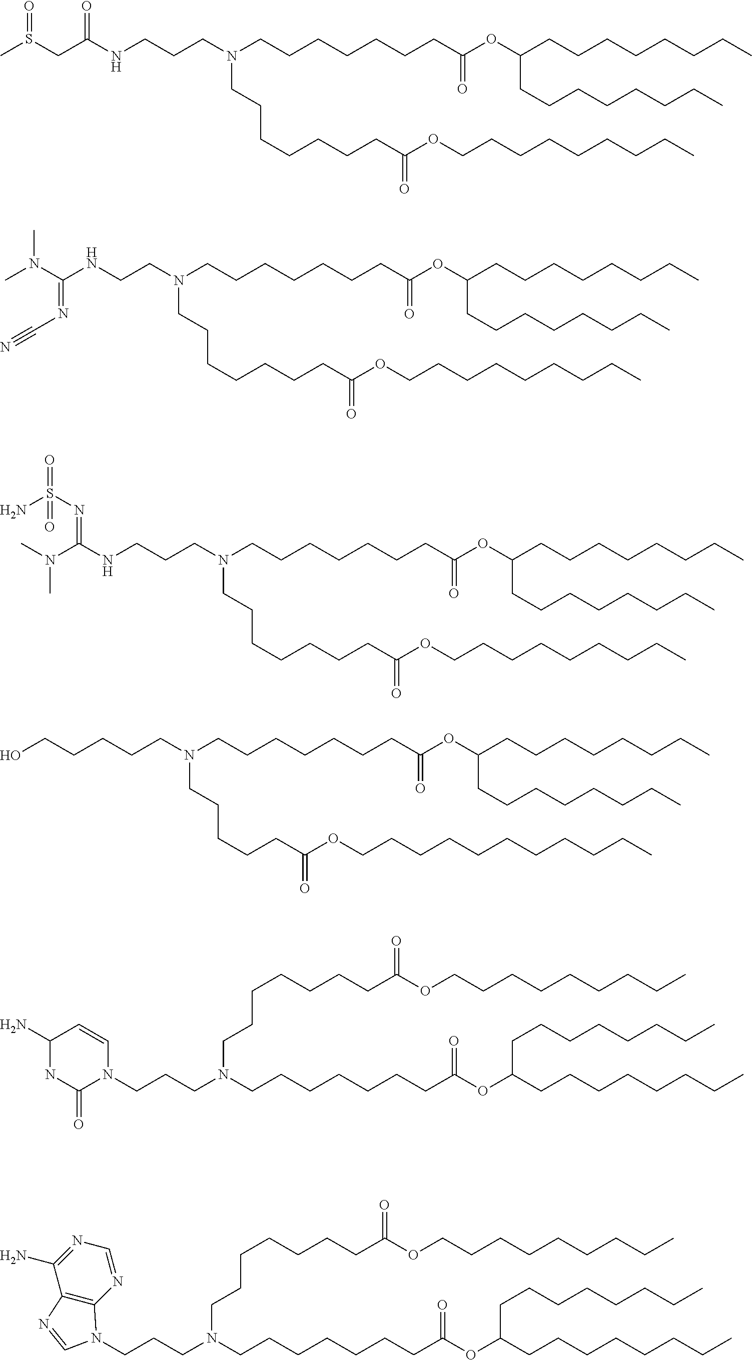

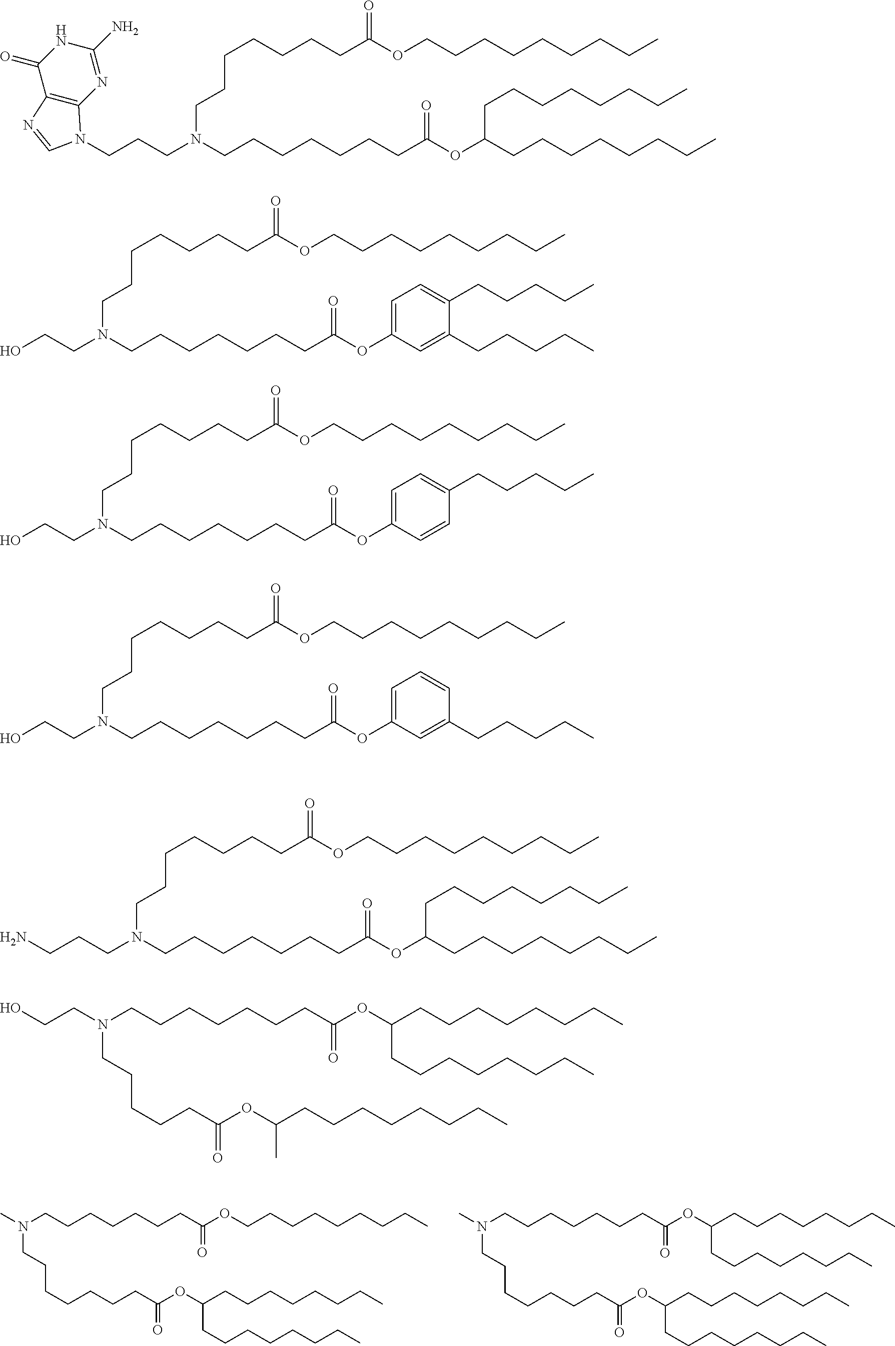

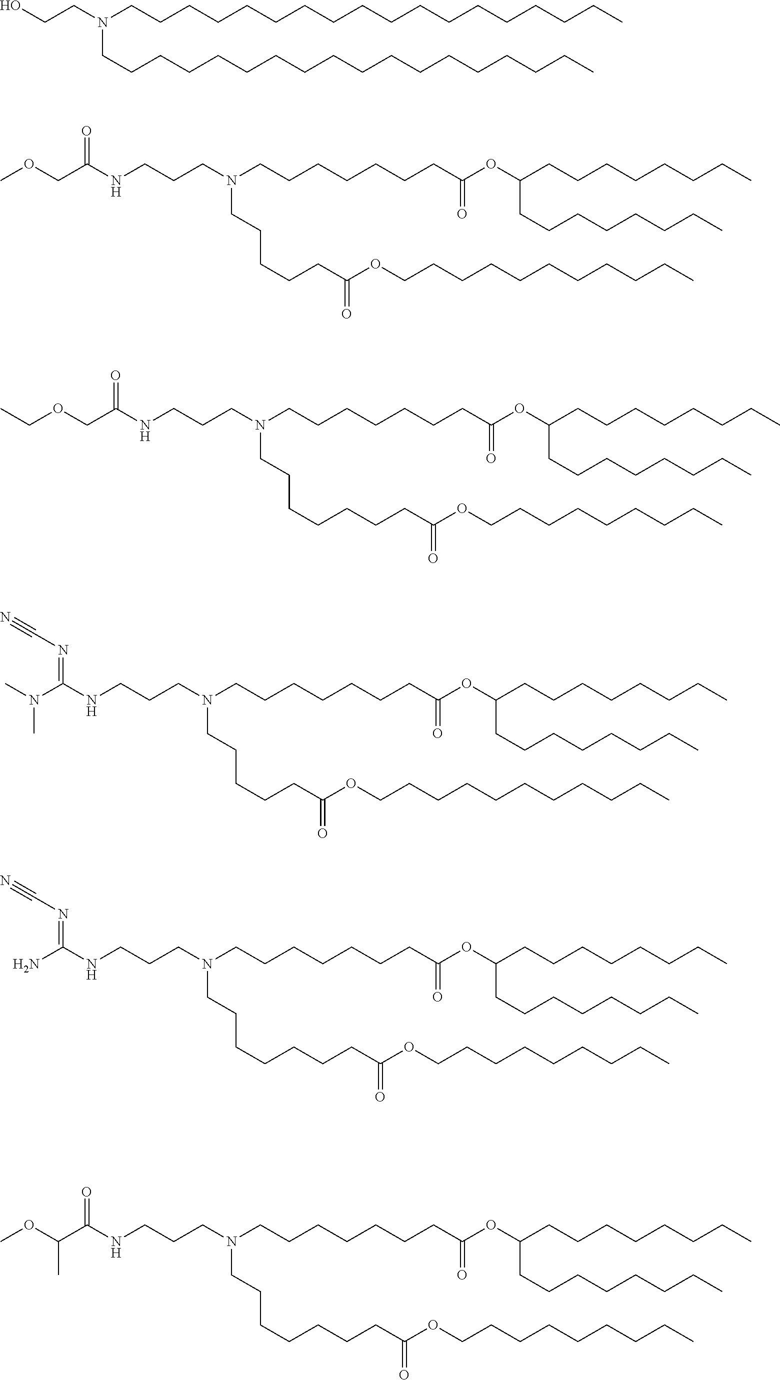

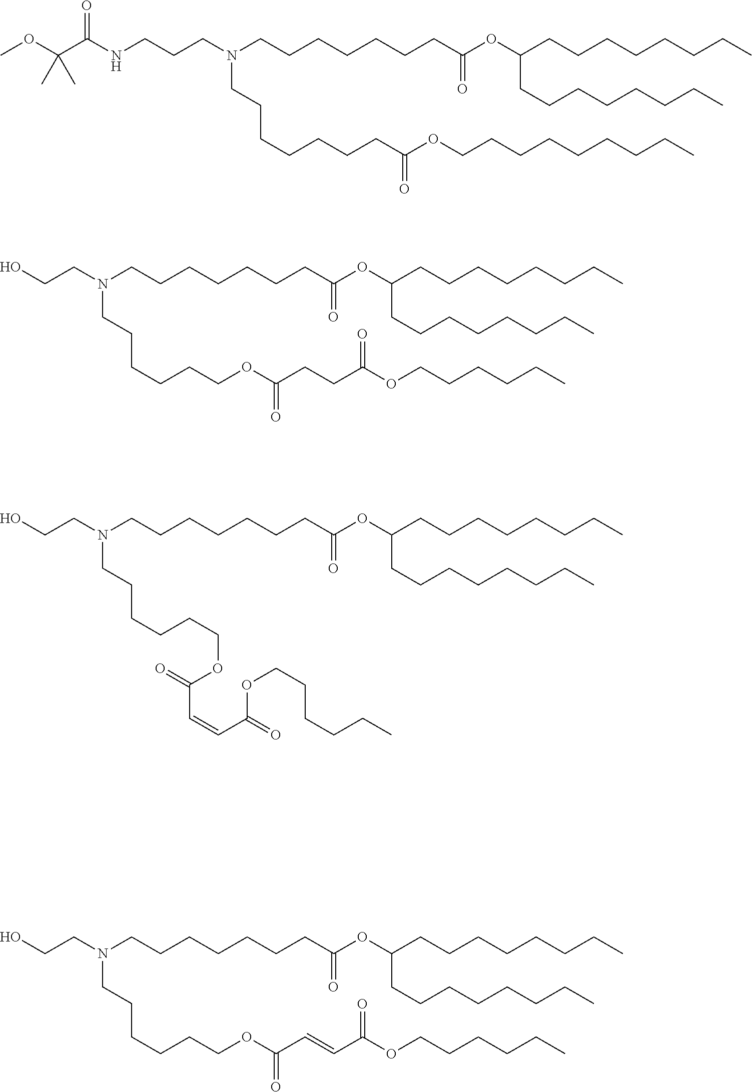 [SMILES: CCCCCCCCCOC(=O)CCCCCCCN(CCCCCCCC(=O)OC(CCCCCCCC)CCCCCCCC)CCCNC(=O)C(C)(C)OC ; CCCCCCCCC(CCCCCCCC)OC(=O)CCCCCCCN(CCO)CCCCCCOC(=O)CCC(=O)OCCCCCC ; CCCCCCCCC(CCCCCCCC)OC(=O)CCCCCCCN(CCO)CCCCCCOC(=O)/C=C\C(=O)OCCCCCC ; CCCCCCCCC(CCCCCCCC)OC(=O)CCCCCCCN(CCO)CCCCCCOC(=O)/C=C/C(=O)OCCCCCC]

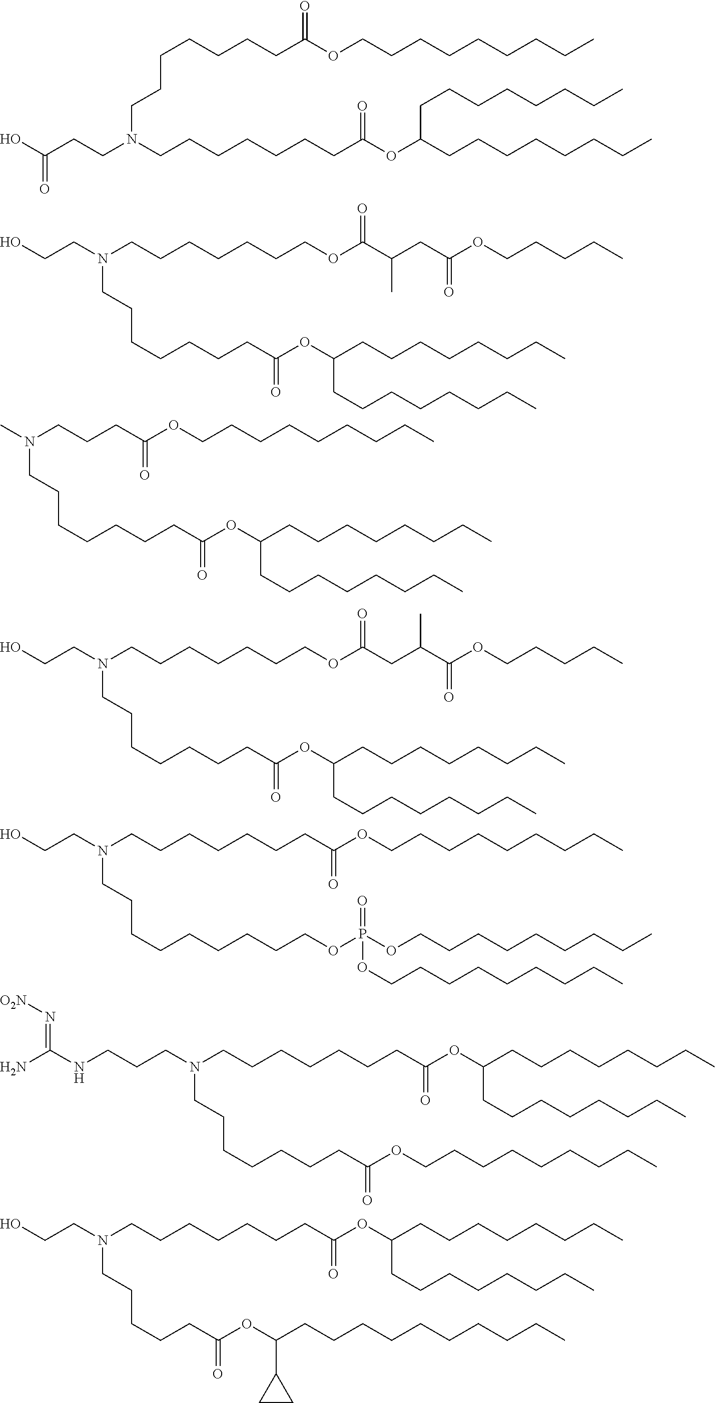

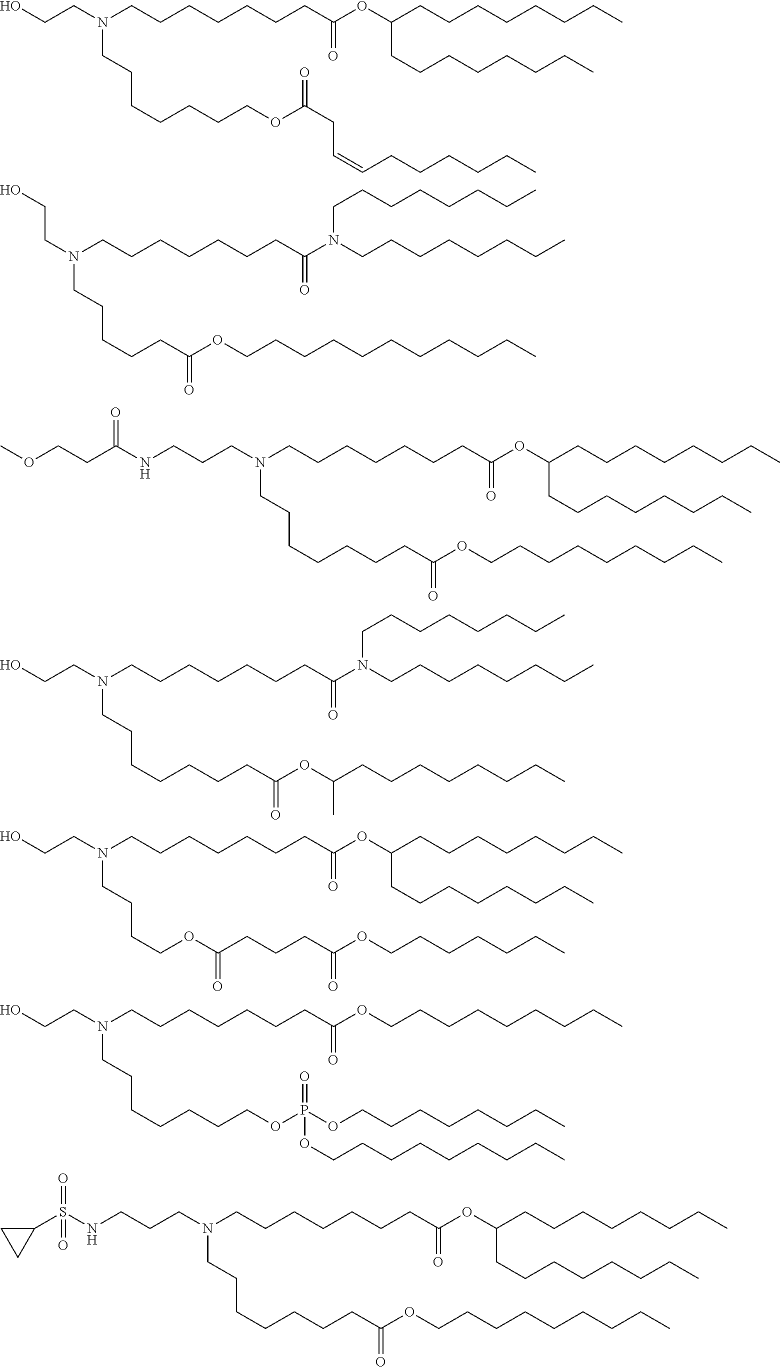

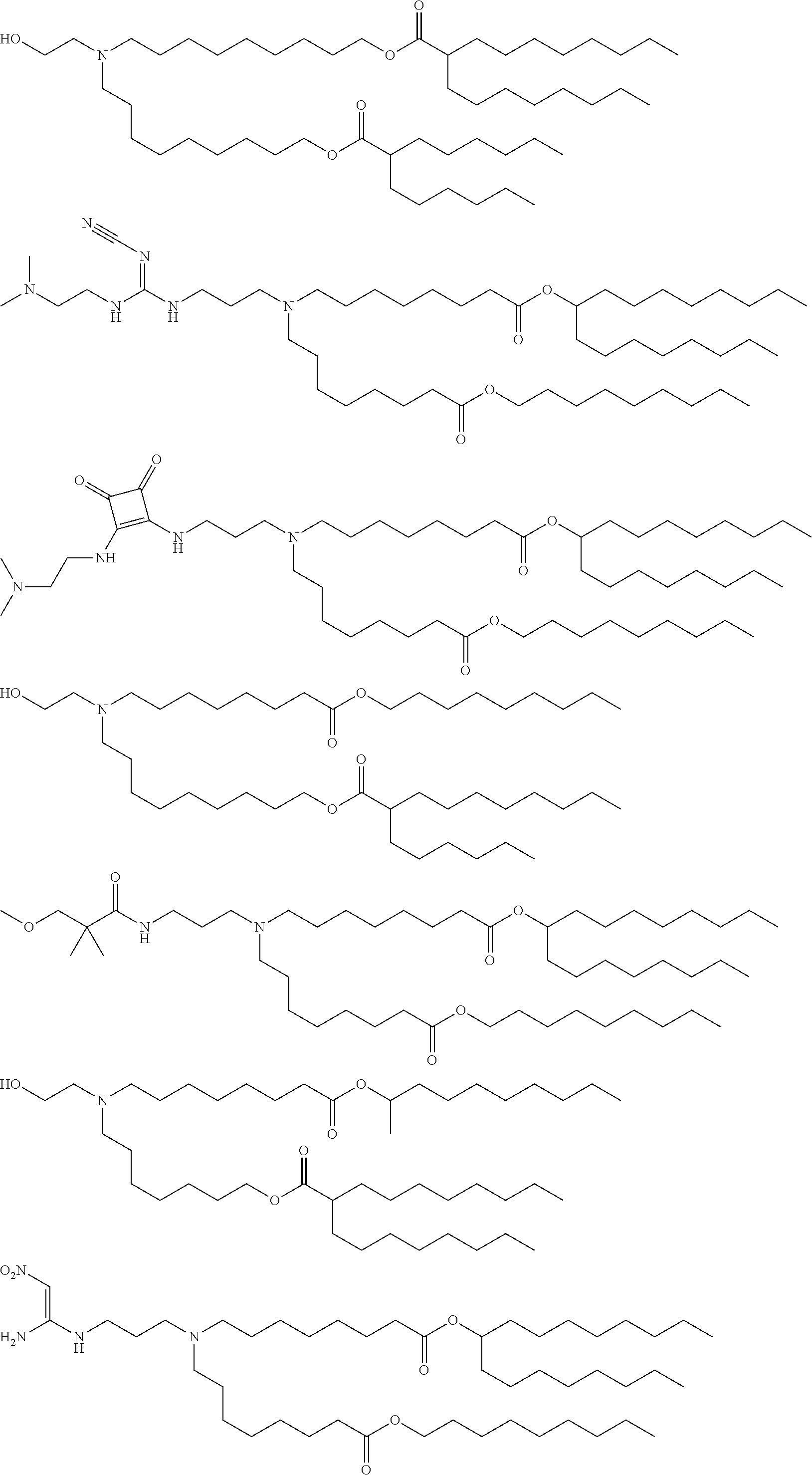

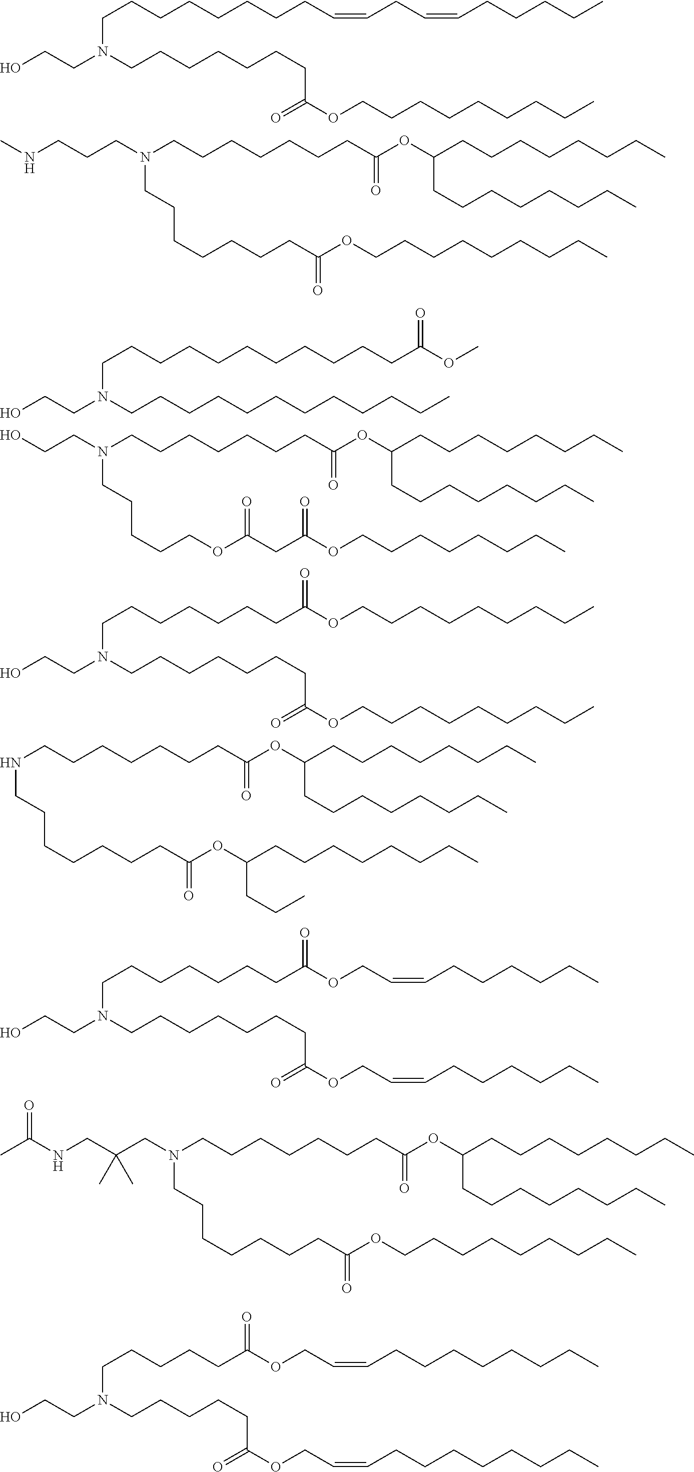

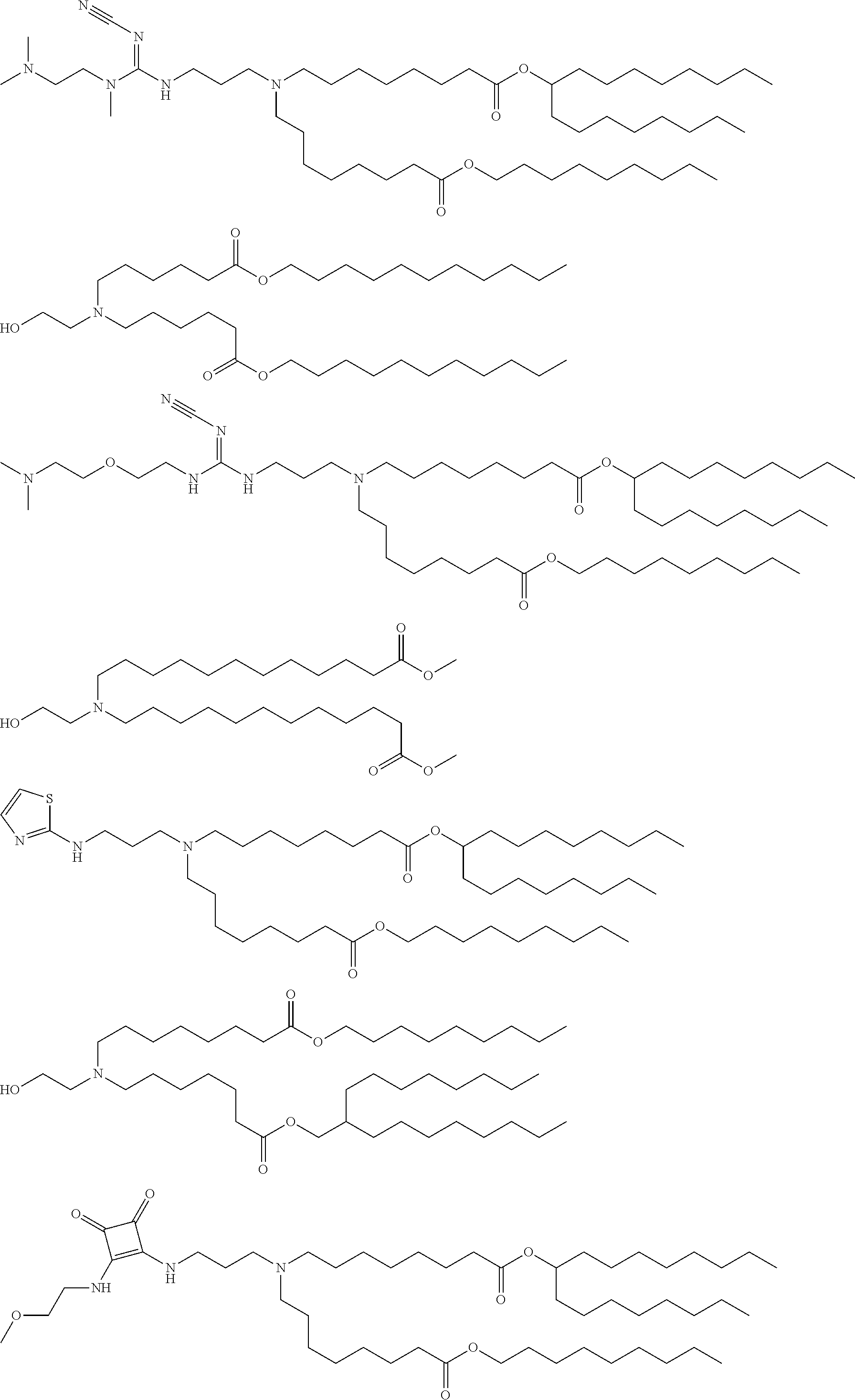

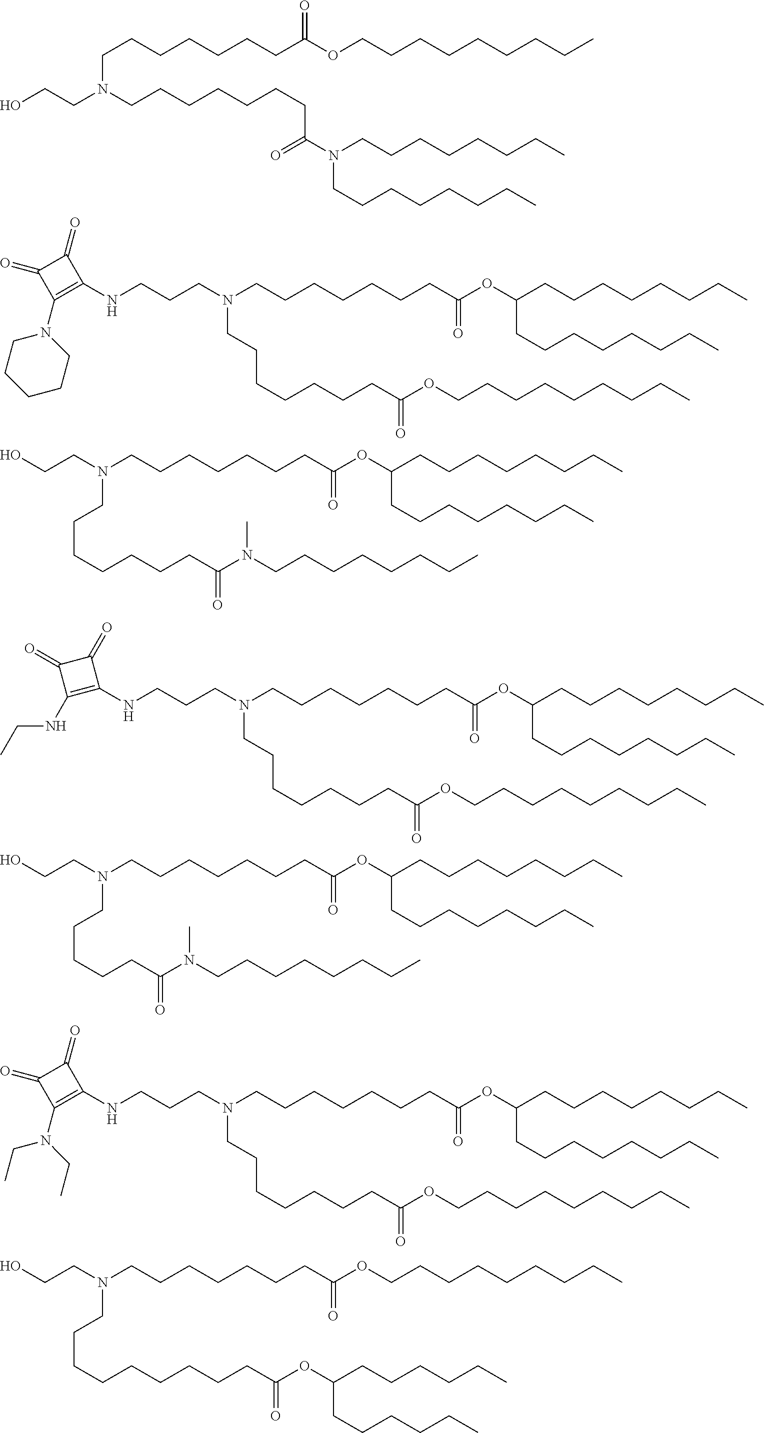

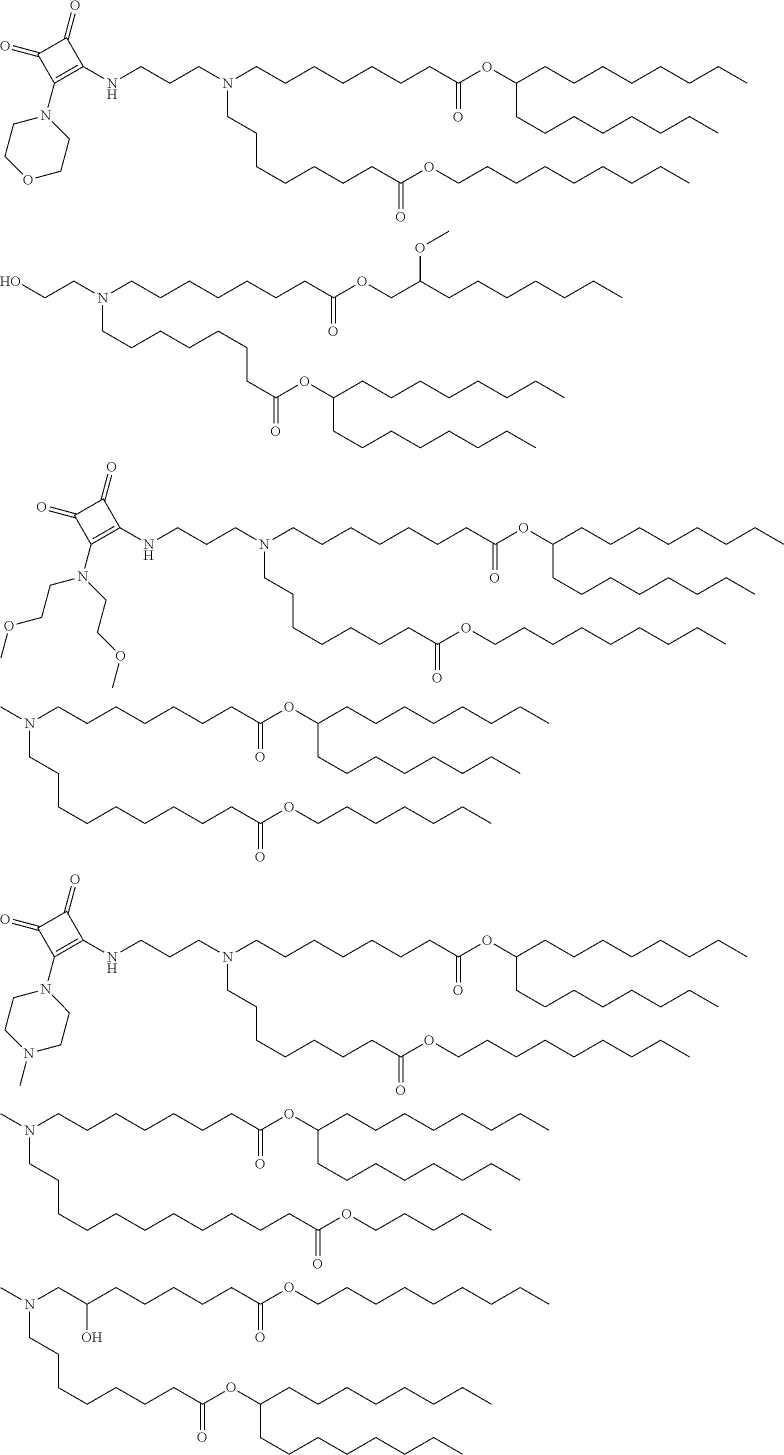

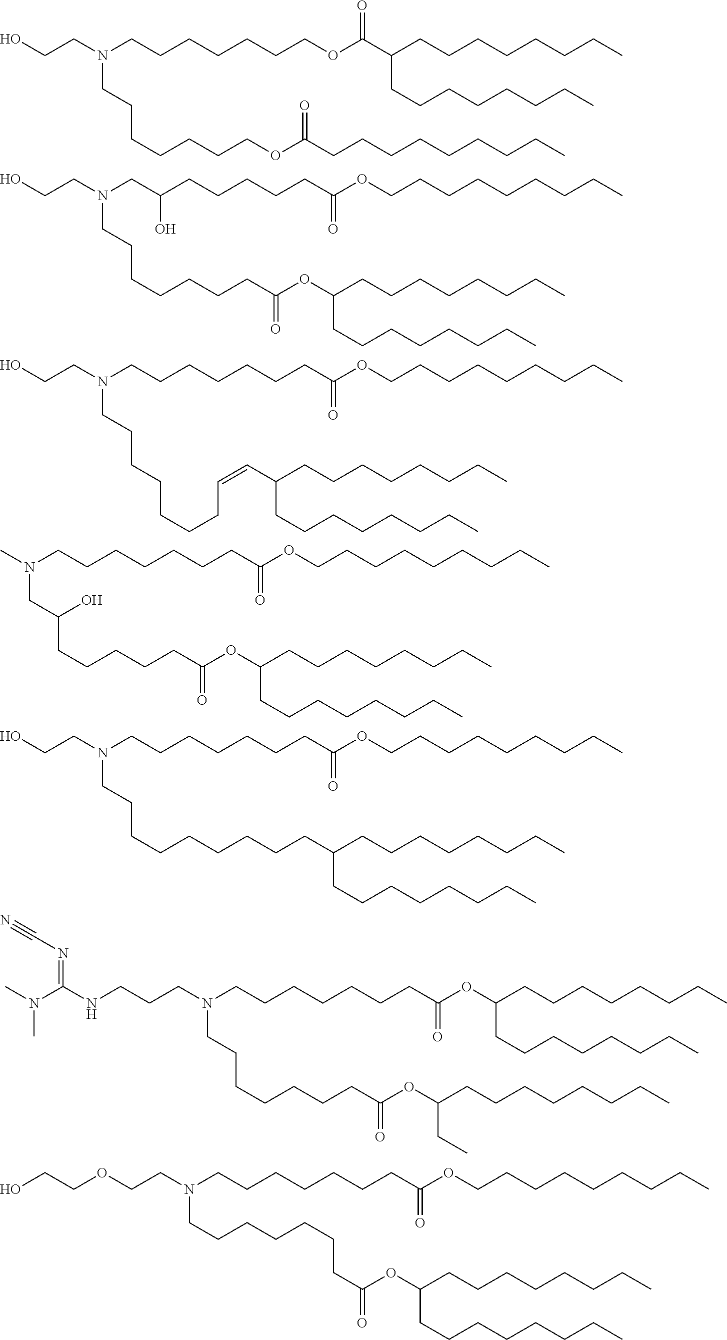

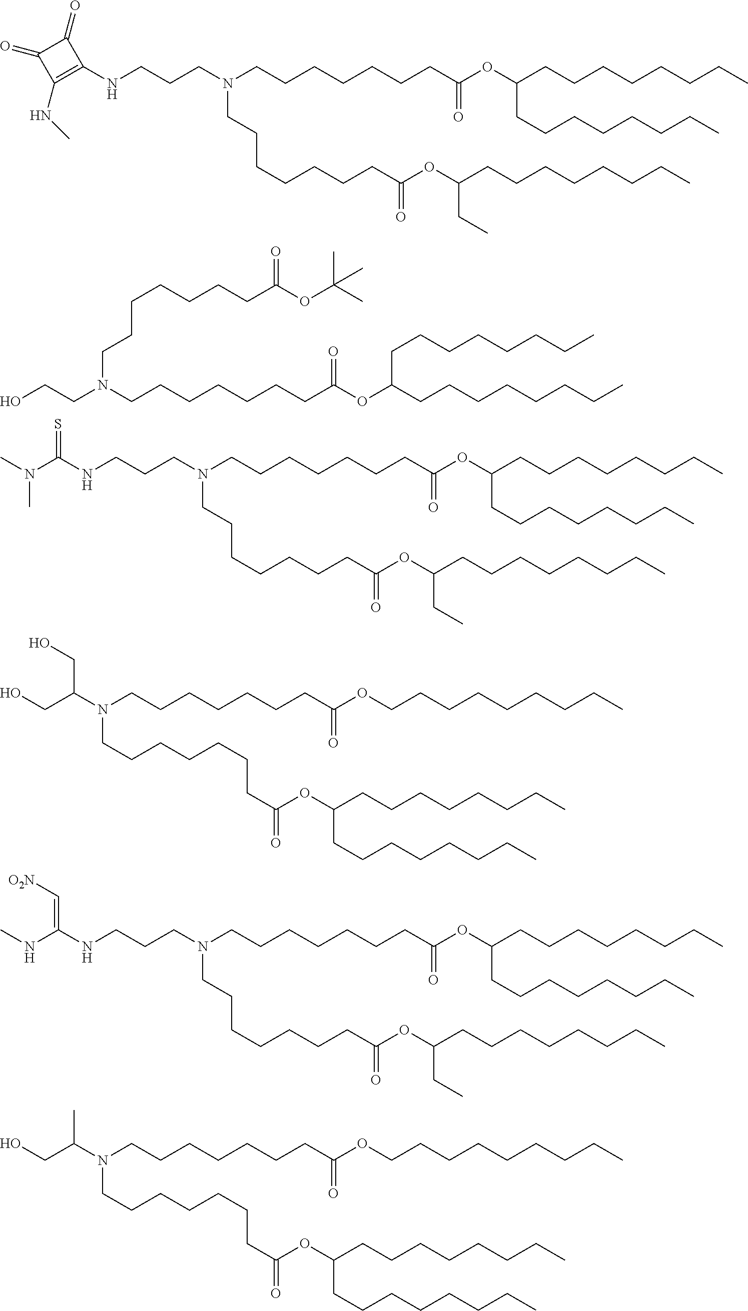

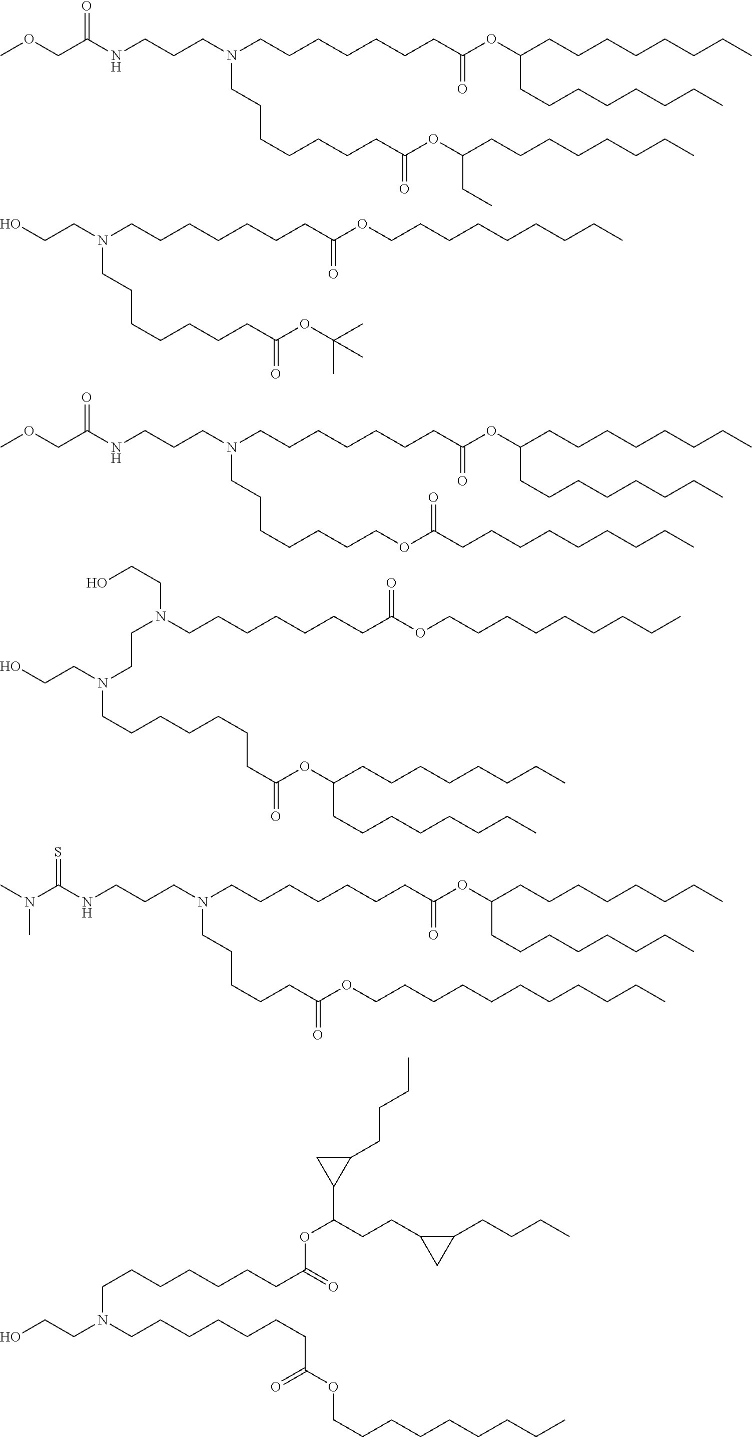

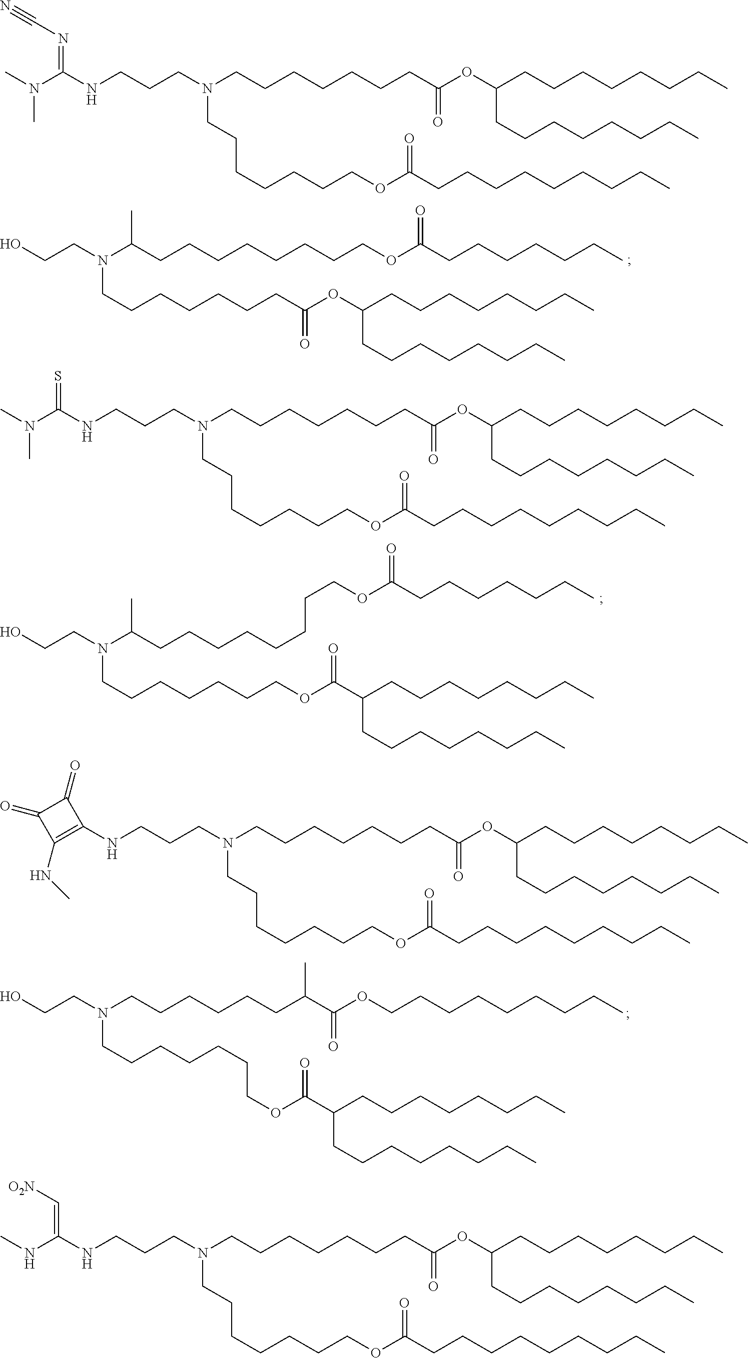

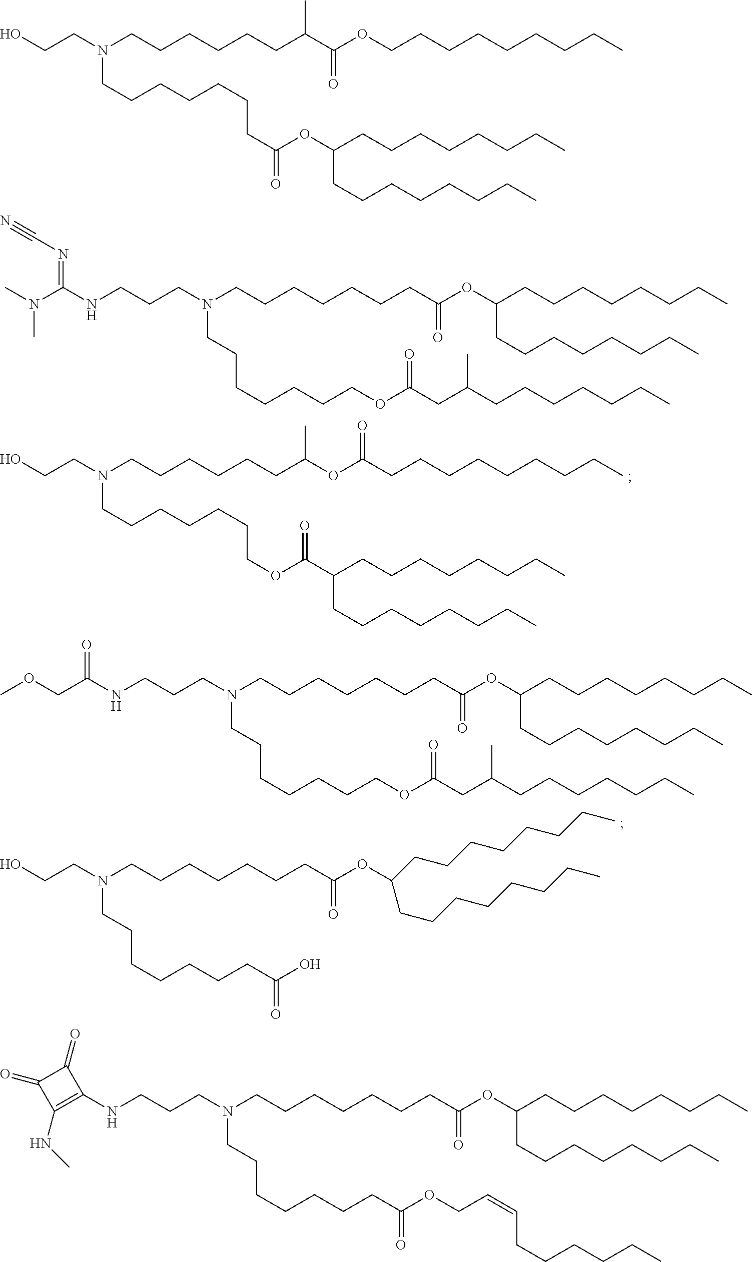

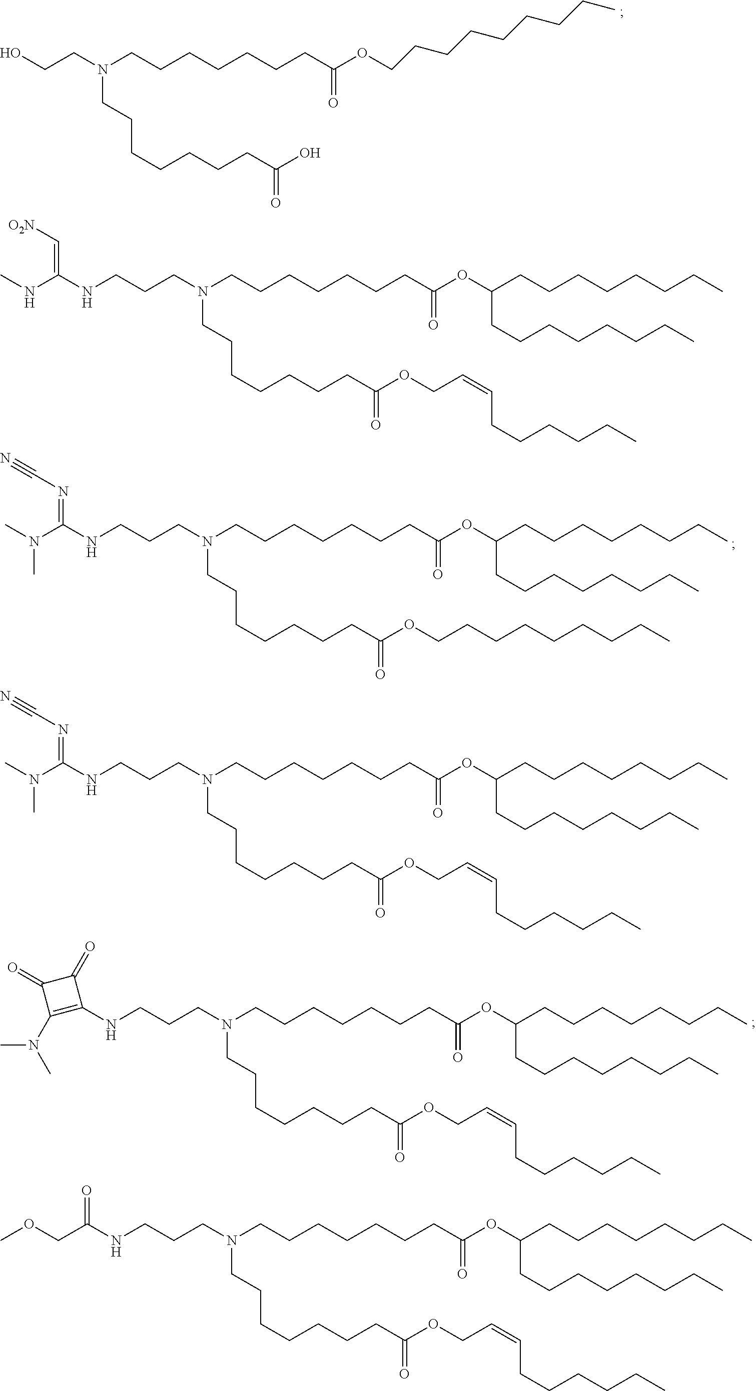

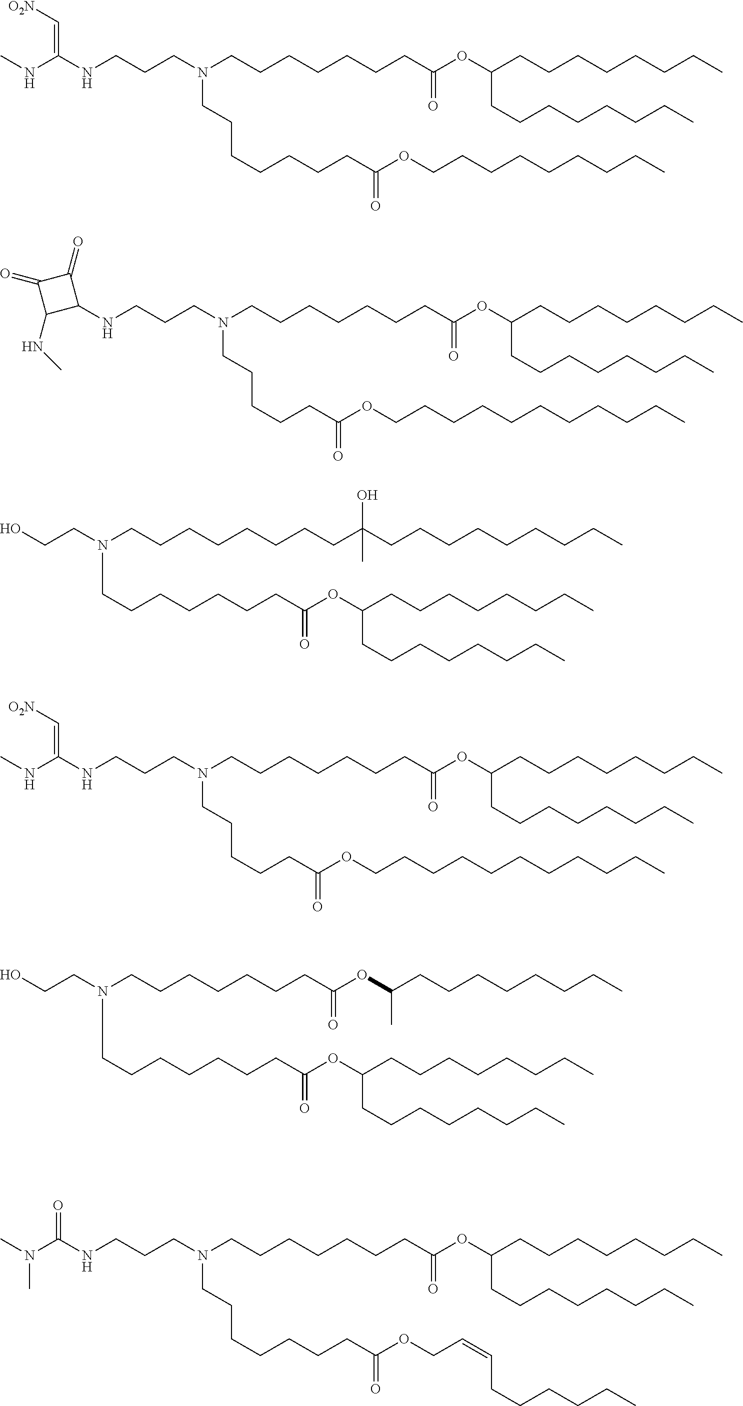

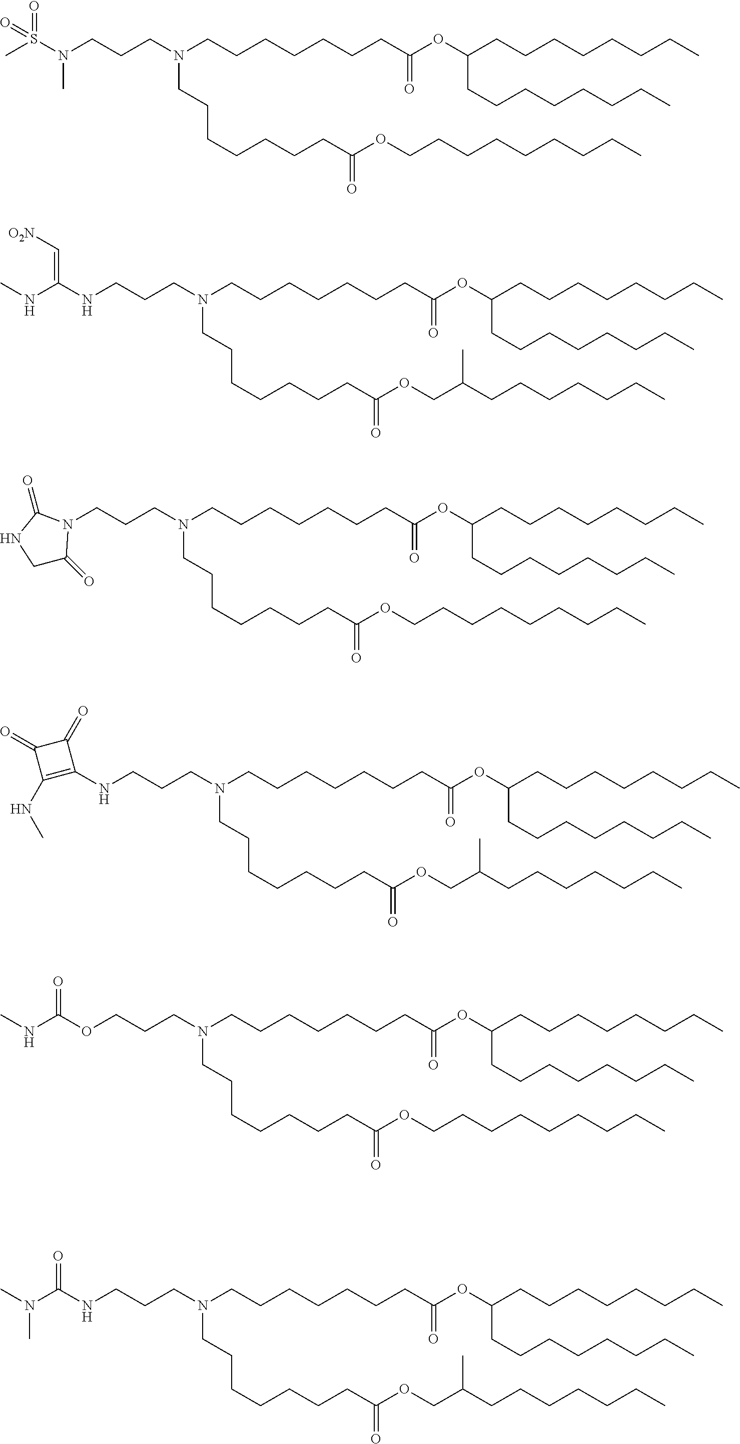

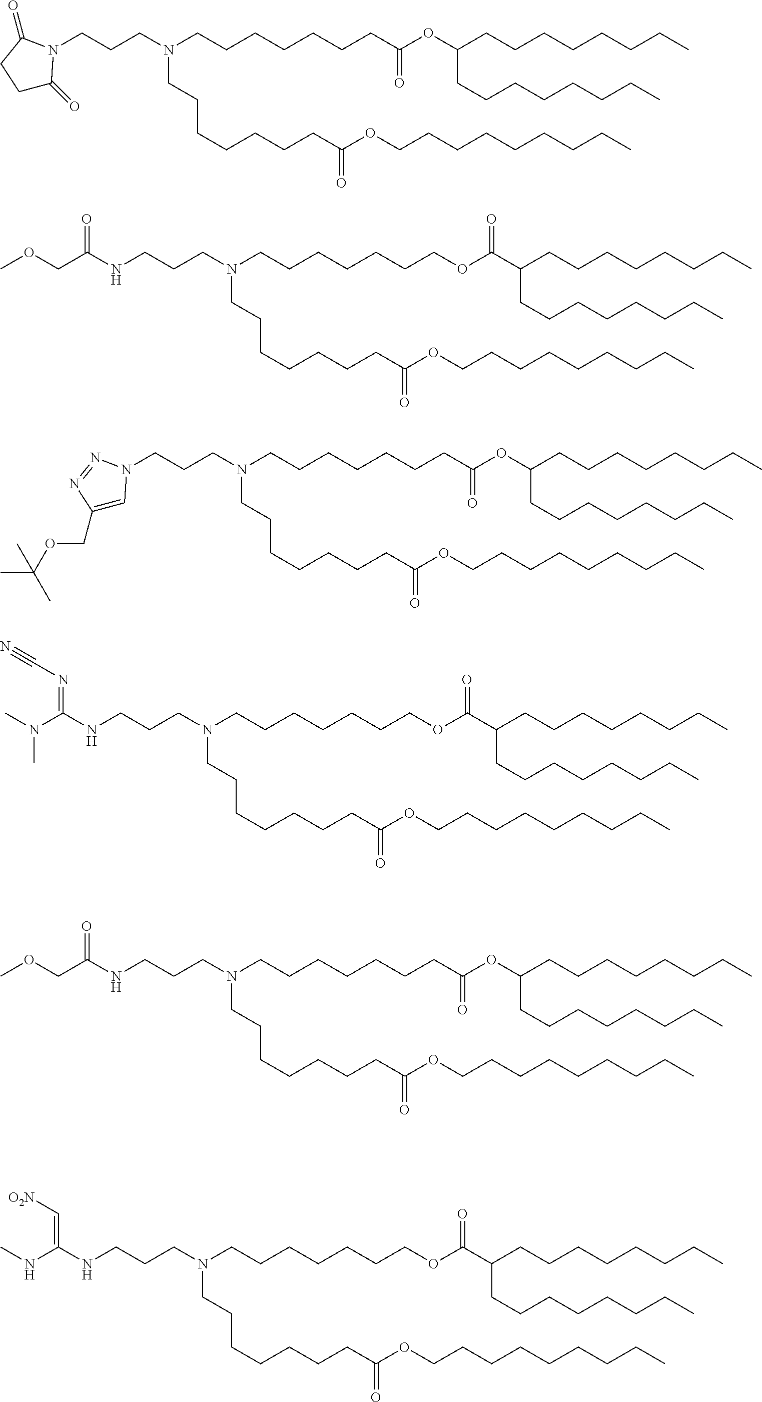

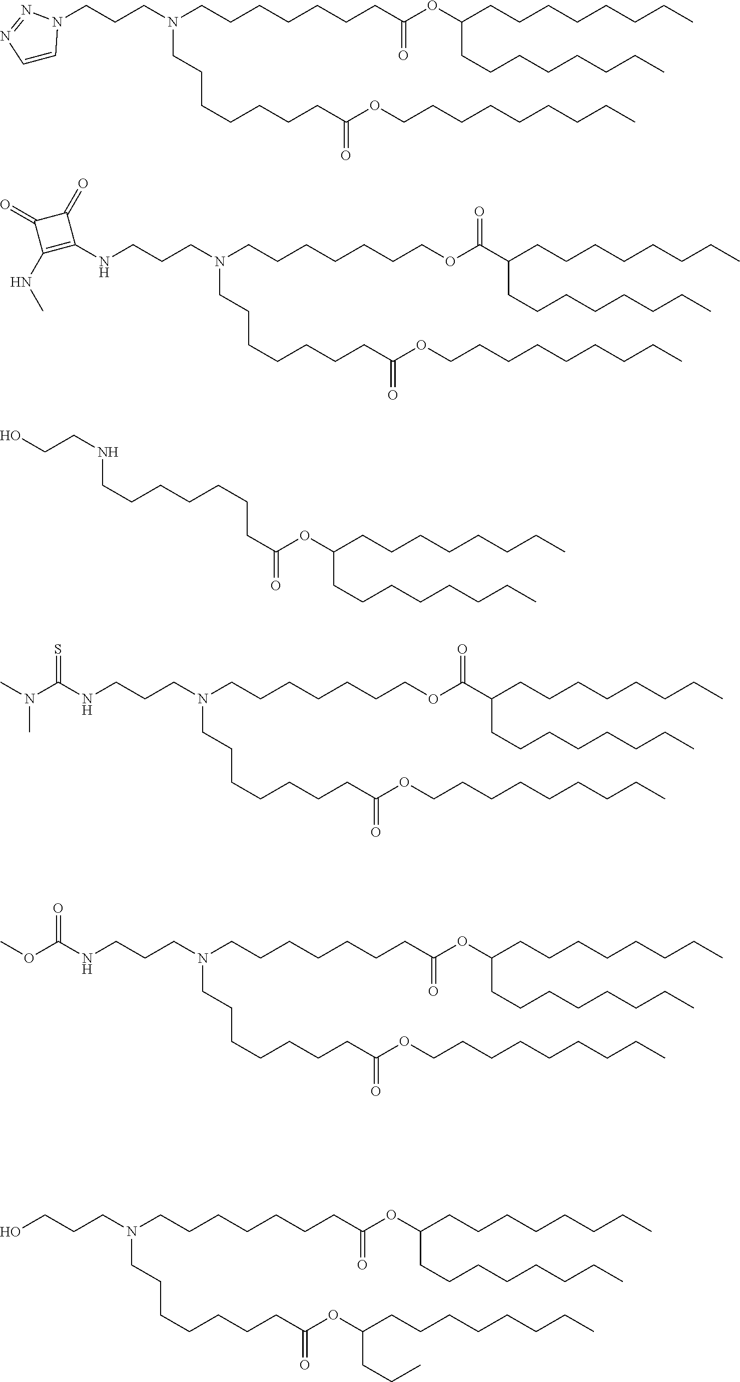

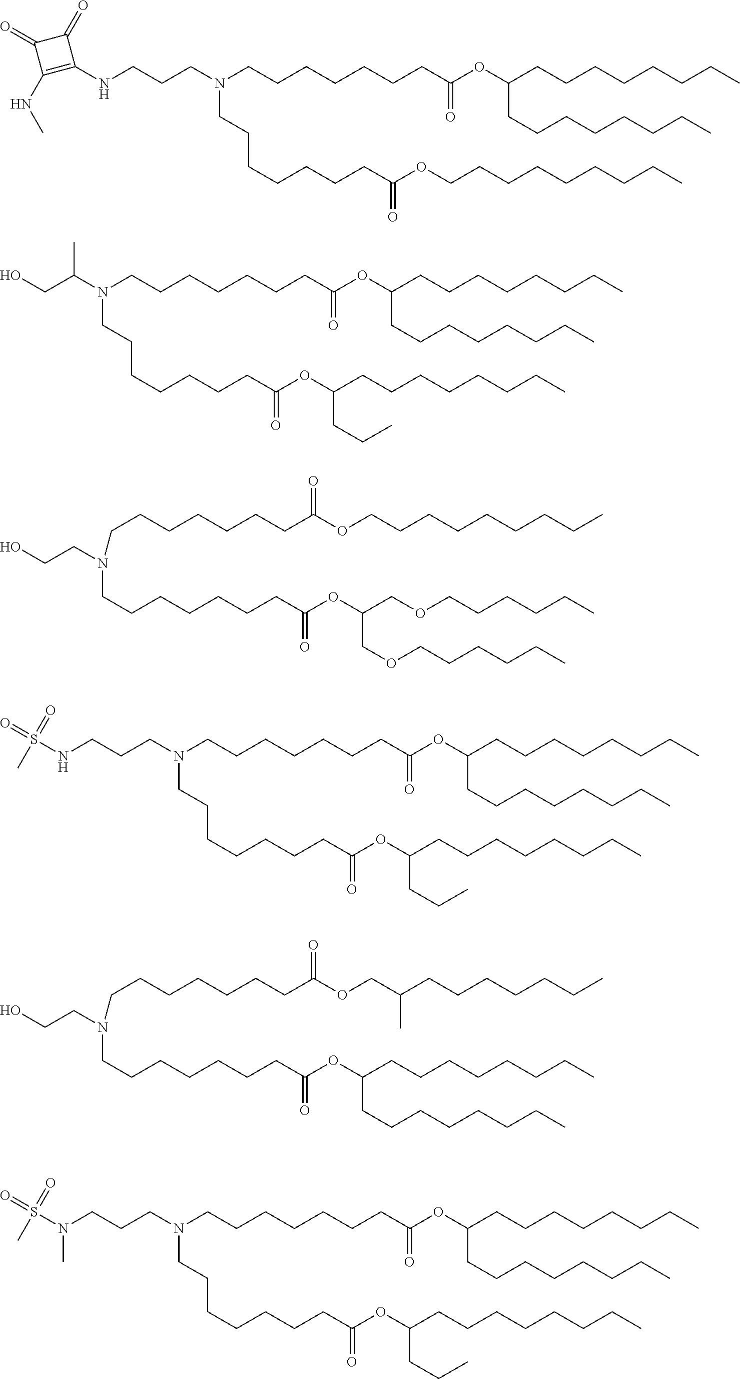 [SMILES: CCCCCCCCCOC(=O)CCCCCCCN(CCCCCCCC(=O)OC(CCCCCCCC)CCCCCCCC)CCCNc1c(NC)c(=O)c1=O ; CCCCCCCCC(CCC)OC(=O)CCCCCCCN(CCCCCCCC(=O)OC(CCCCCCCC)CCCCCCCC)C(C)CO ; CCCCCCCCCOC(=O)CCCCCCCN(CCO)CCCCCCCC(=O)OC(COCCCCCC)COCCCCCC ; CCCCCCCCC(CCC)OC(=O)CCCCCCCN(CCCCCCCC(=O)OC(CCCCCCCC)CCCCCCCC)CCCNS(C)(=O)=O ; CCCCCCCCC(CCCCCCCC)OC(=O)CCCCCCCN(CCO)CCCCCCCC(=O)OCC(C)CCCCCCC ; CCCCCCCCC(CCC)OC(=O)CCCCCCCN(CCCCCCCC(=O)OC(CCCCCCCC)CCCCCCCC)CCCN(C)S(C)(=O)=O]

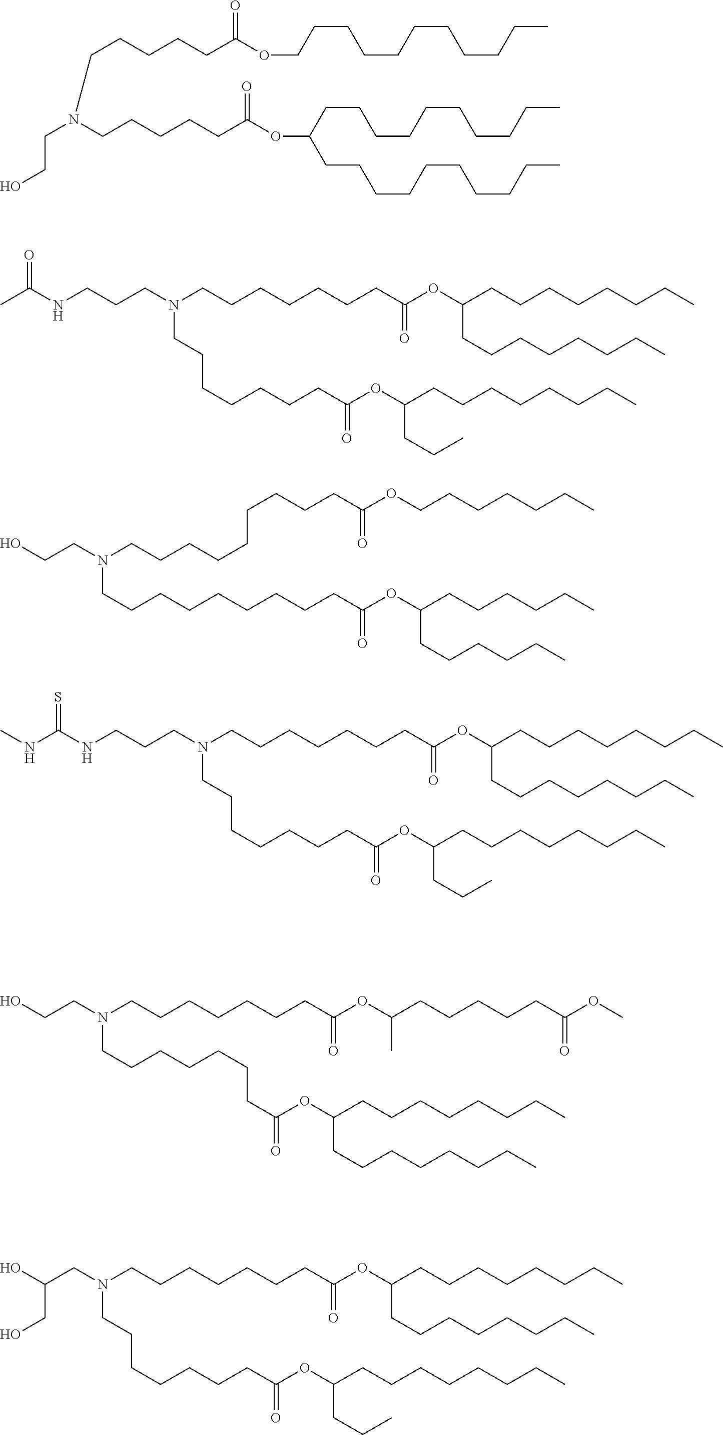

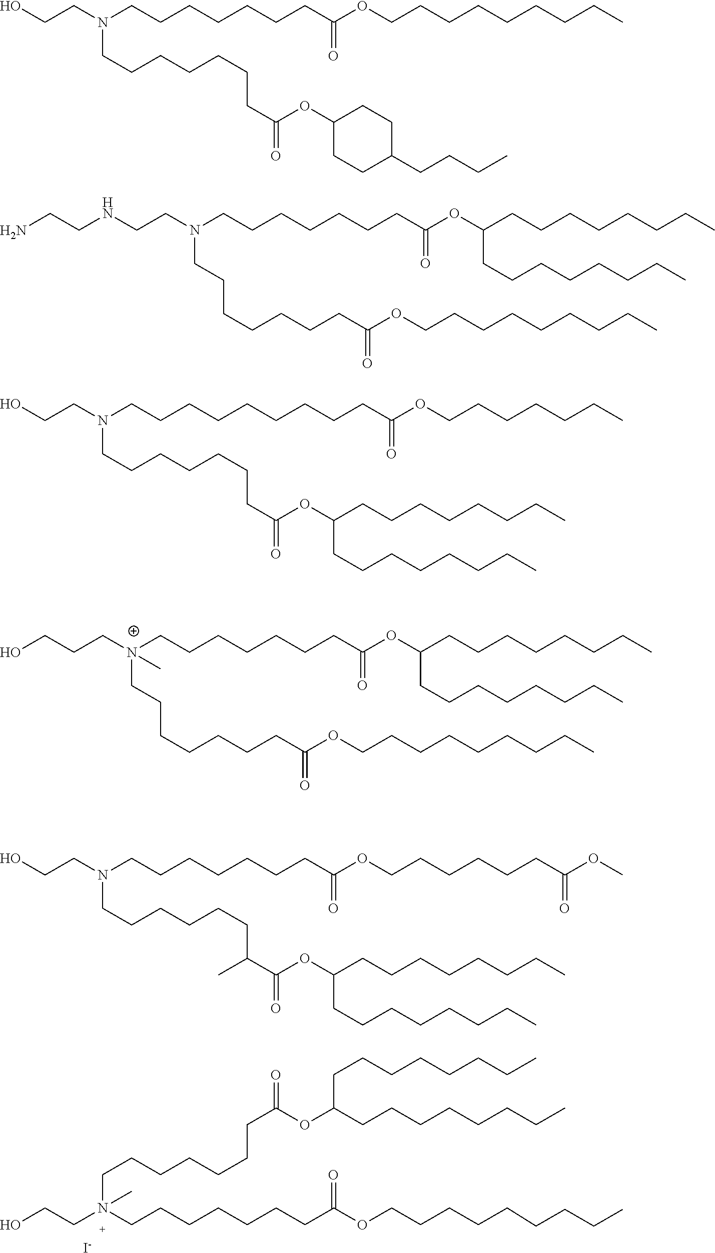

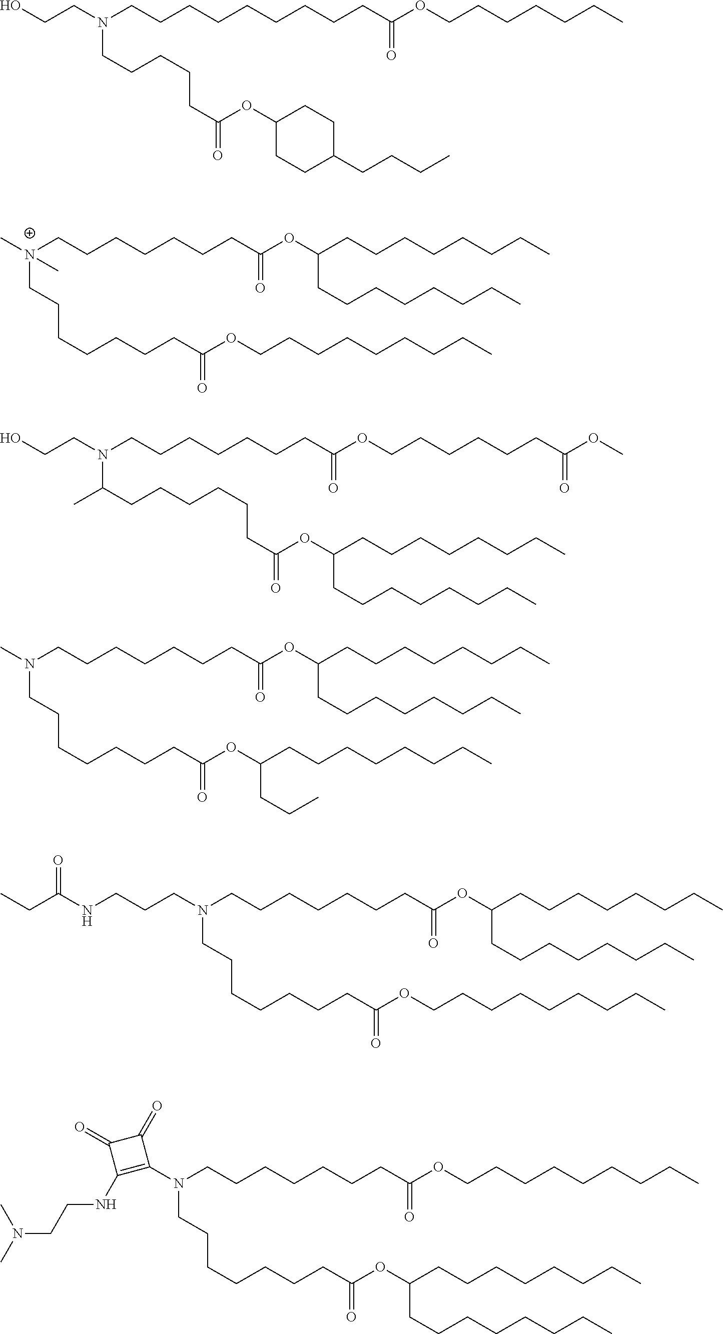

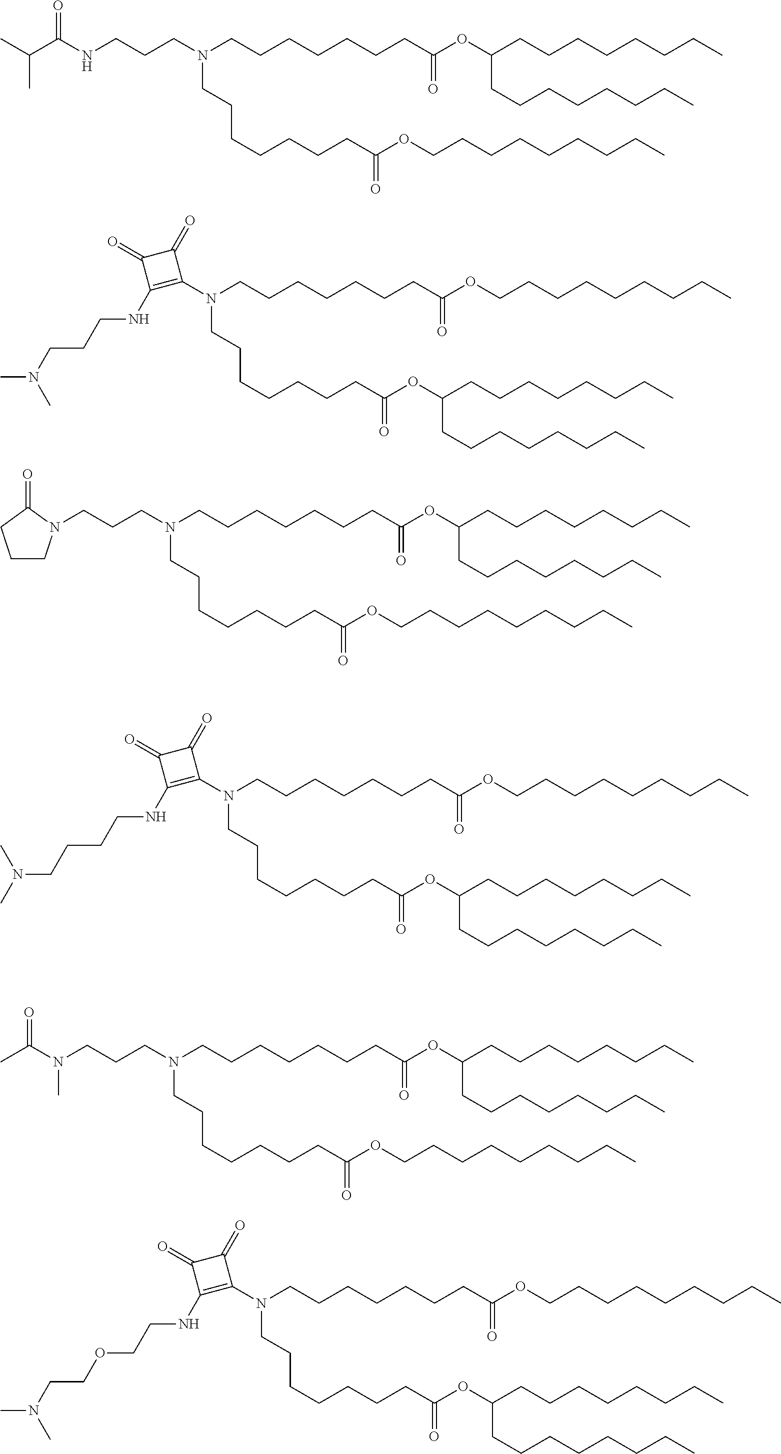

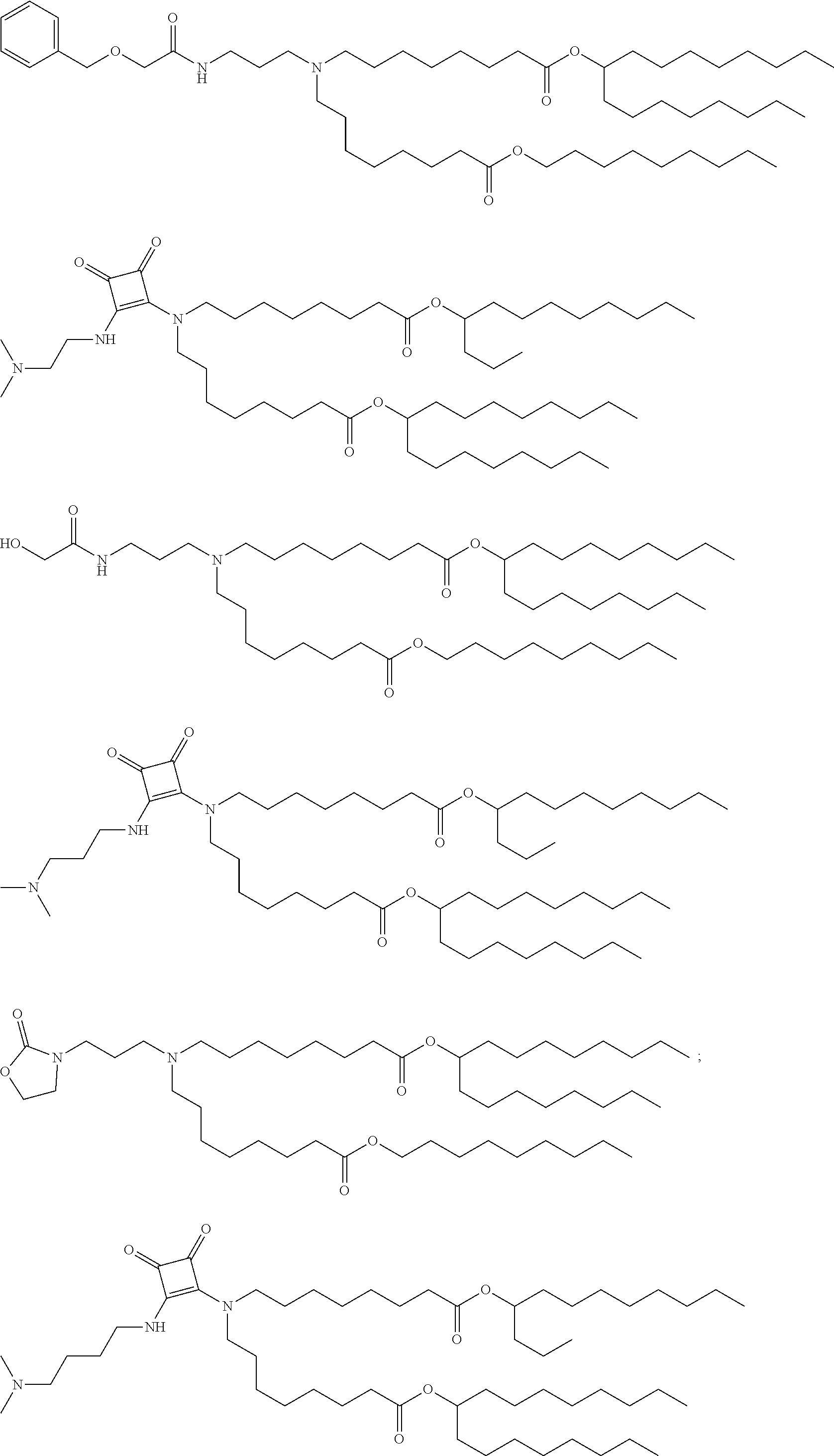

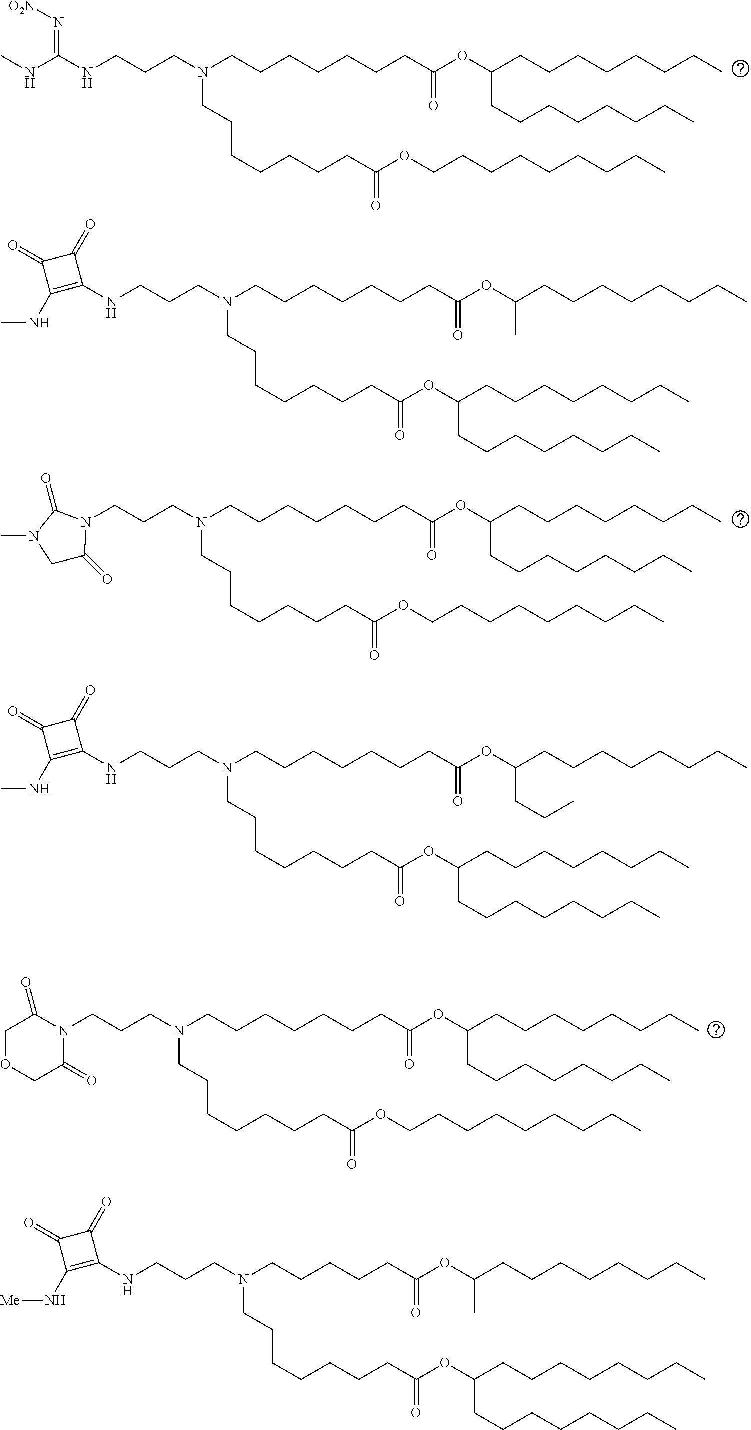

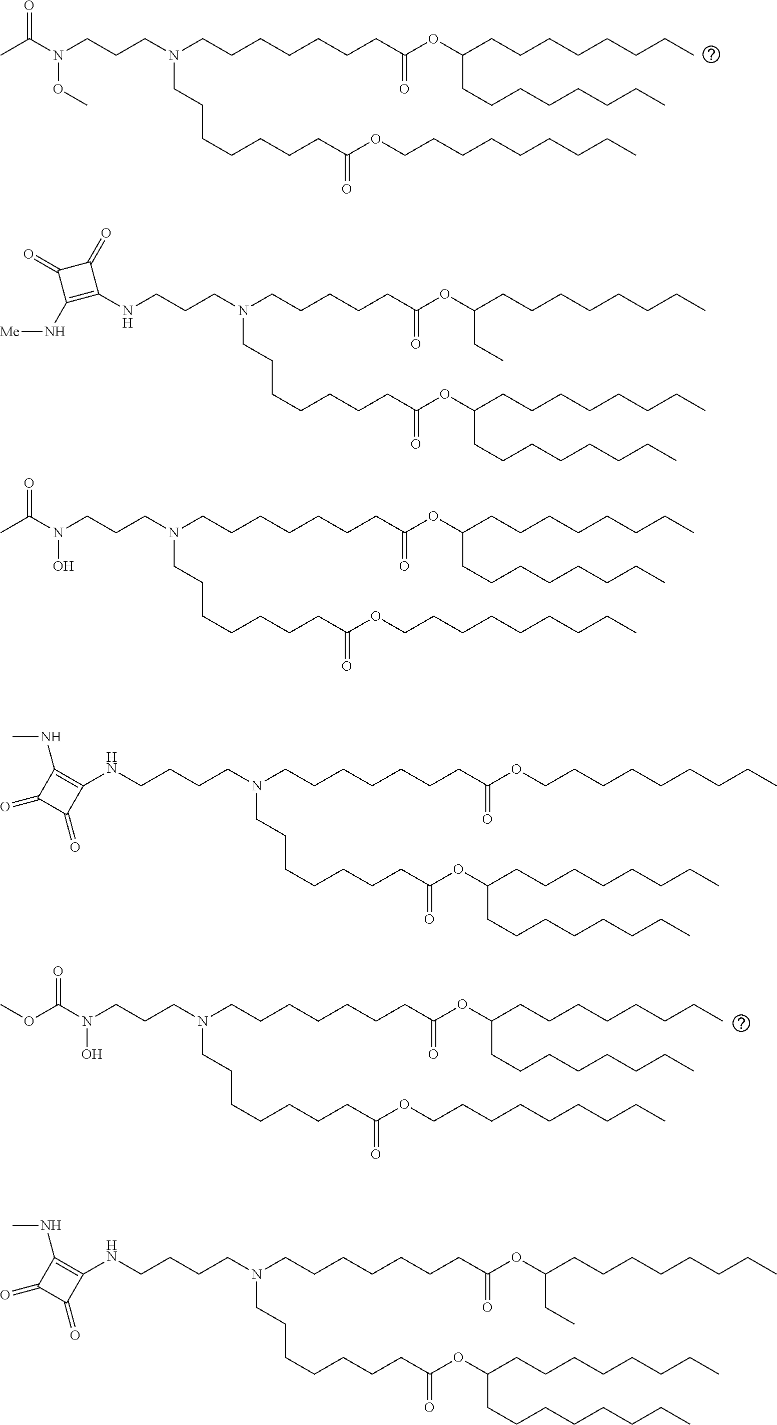

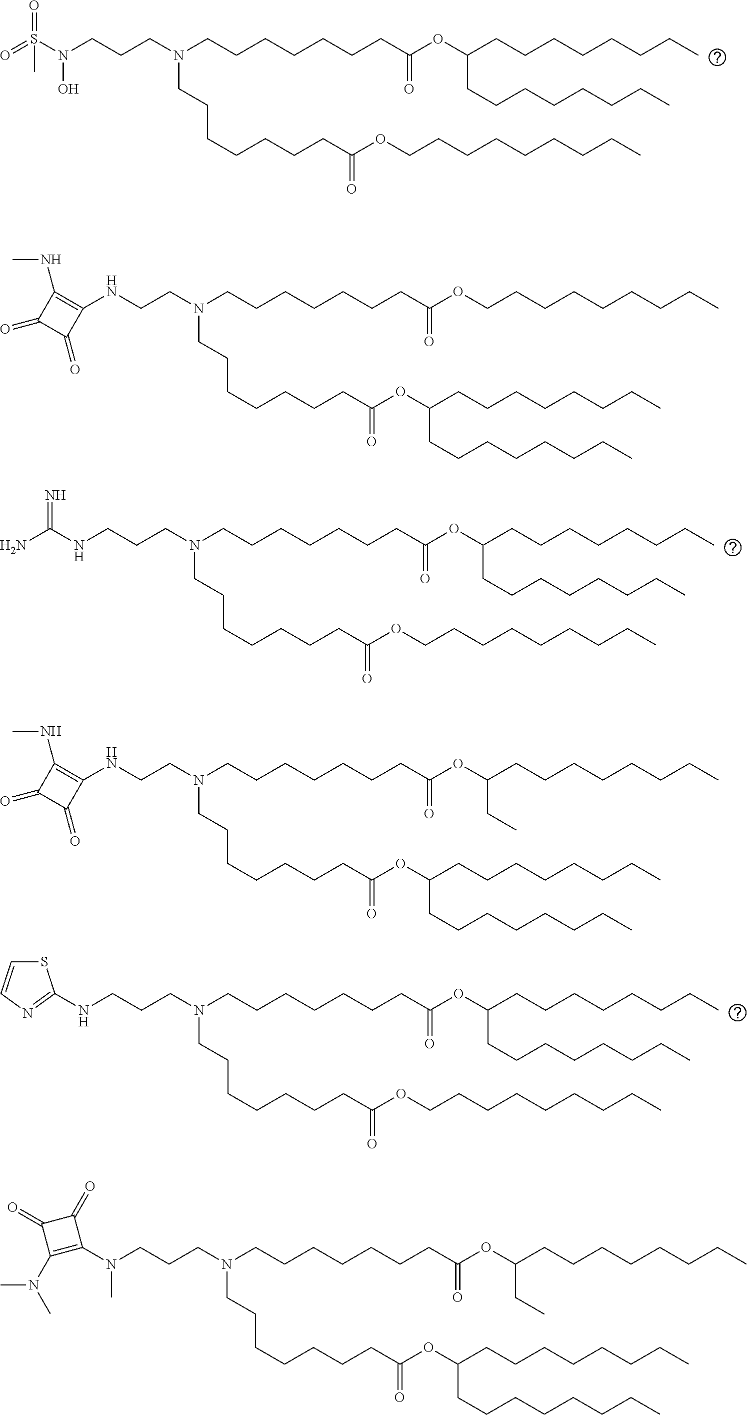

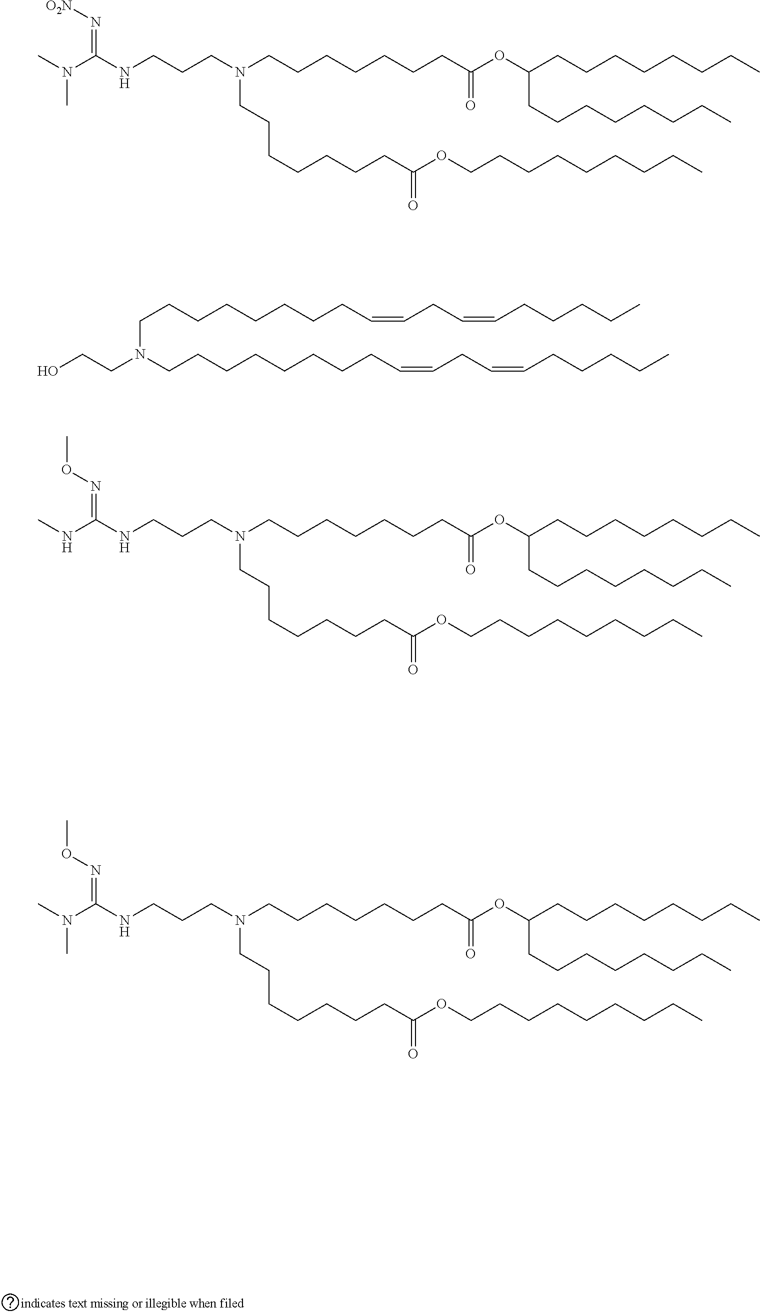

In some embodiments, an ionizable lipid has a disulfide tail.

In some embodiments, an ionizable lipid includes short peptides of 12-15 mer length as head groups.

In some embodiments, the head of an ionizable lipid comprises the structure of Vitamin A, D, E, or K as described in the published Patent Application WO2019232095A1, which is incorporated by herein by reference in its entirety.

In some embodiments, a pharmaceutical composition described herein is capable of delivering a payload to a target organ. In some embodiments, a pharmaceutical composition described herein delivers a higher proportion of its payload to a target organ than a reference pharmaceutical composition does. In some embodiments, a pharmaceutical composition delivers at least about 10%, 20%, 30%, 40%, 50%, 60%, 70%, 80%, 90%, 100%, 150%, 200%, 250%, or 300% more payload to a target organ than a reference pharmaceutical composition does. In some embodiments, the ratio of payload delivered to a target organ with the payload delivered to the liver is higher for a pharmaceutical composition described herein than a reference pharmaceutical composition. In some embodiments, the ratio of payload delivered to a target organ with the payload delivered to the liver is at least about 10%, 20%, 30%, 40%, 50%, 60%, 70%, 80%, 90%, 100%, 150%, 200%, 250%, or 300% higher for a pharmaceutical composition described herein than a reference pharmaceutical composition. In some embodiments, a reference pharmaceutical composition contains MC3, KC2, or DLinDMA. In some embodiments, a reference pharmaceutical composition contains about 50 mol % MC3, about 10 mol % DSPC, about 38.5 mol % cholesterol, and about 1.5 mol % PEG-DMG.

In some embodiments, a lipid is described in international patent applications WO2021077067, or WO2019152557, each of which is incorporated herein by reference in its entirety.

In some embodiments, a LNP described herein comprises a lipid, e.g., an ionizable lipid, disclosed in US 2019/0240354, which is incorporated herein by reference in its entirety.

In some embodiments, the lipids disclosed in US 2019/0240354 are of Formula I:

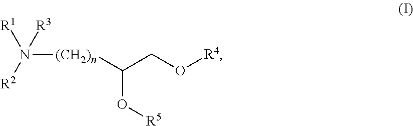

or salts thereof, wherein:
R¹ and R² are either the same or different and are independently hydrogen (H) or an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof;
R³ is either absent or is hydrogen (H) or a C₁-C₆ alkyl to provide a quaternary amine; R⁴ and R⁵ are either the same or different and are independently an optionally substituted C₁₀-C₂₄ alkyl, C₁₀-C₂₄ alkenyl, C₁₀-C₂₄ alkynyl, or C₁₀-C₂₄ acyl, wherein at least one of R⁴ and R⁵ comprises at least two sites of unsaturation; and
n is 0, 1, 2, 3, or 4.

In some embodiments, the lipids disclosed in US 2019/0240354 are of Formula II:

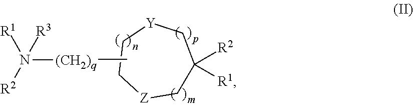

wherein R¹ and R² are either the same or different and are independently an optionally substituted C₁₂-C₂₄ alkyl, C₁₂-C₂₄ alkenyl, C₁₂-C₂₄ alkynyl, or C₁₂-C₂₄ acyl; R³ and R⁴ are either the same or different and are independently an optionally substituted C₁-C₆ alkyl, C₂-C₆ alkenyl, or C₂-C₆ alkynyl, or R³ and R⁴ may join to form an optionally substituted heterocyclic ring of 4 to 6 carbon atoms and 1 or 2 heteroatoms chosen from nitrogen and oxygen; R⁵ is either absent or is hydrogen (H) or a C₁-C₆ alkyl to provide a quaternary amine; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH. In some embodiments, q is 2.

In some embodiments, the cationic lipid of Formula II is 2,2-dilinoleyl-4-(2-dimethylaminoethyl)-[1,3]-dioxolane, 2,2-dilinoleyl-4-(3-dimethylaminopropyl)-[1,3]-dioxolane, 2,2-dilinoleyl-4-(4-dimethylaminobutyl)-[1,3]-dioxolane, 2,2-dilinoleyl-5-dimethylaminomethyl-[1,3]-dioxane, 2,2-dilinoleyl-4-N-methylpepiazino-[1,3]-dioxolane, 2,2-dilinoleyl-4-dimethylaminomethyl-[1,3]-dioxolane, 2,2-dioleoyl-4-dimethylaminomethyl-[1,3]-dioxolane, 2,2-distearoyl-4-dimethylaminomethyl-[1,3]-dioxolane, 2,2-dilinoleyl-4-N-morpholino-[1,3]-dioxolane, 2,2-Dilinoleyl-4-trimethylamino-[1,3]-dioxolane chloride, 2,2-dilinoleyl-4,5-bis(dimethylaminomethyl)-[1,3]-dioxolane, 2,2-dilinoleyl-4-methylpiperzine-[1,3]-dioxolane, or mixtures thereof. In some embodiments, the cationic lipid of Formula II is 2,2-dilinoleyl-4-(2-dimethylaminoethyl)-[1,3]-dioxolane.

In some embodiments, the lipids disclosed in US 2019/0240354 are of Formula III:

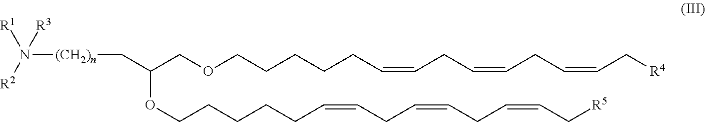

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either absent or present and when present are either the same or different and are independently an optionally substituted C₁-C₁₀ alkyl or C₂-C₁₀ alkenyl; and n is 0, 1, 2, 3, or 4.

In some embodiments, the lipids disclosed in US 2019/0240354 are of Formula C:

X-A-Y—Z¹; (Formula C)

or salts thereof, wherein:

X is —N(H)R or —NR₂;

A is absent, C₁ to C₆ alkyl, C₂ to C₆ alkenyl, or C₂ to C₆ alkynyl, which C₁ to C₆ alkyl, C₂ to C₆ alkenyl, and C₂ to C₆ alkynyl is optionally substituted with one or more groups independently selected from oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}, wherein n is 0, 1, or 2, and R^{x} and R^{y} are each independently hydrogen, alkyl, or heterocycle, wherein each alkyl and heterocycle of R^{x} and R^{y} may be further substituted with one or more groups independently selected from oxo, halogen, —OH, —CN, alkyl, —OR^{x′}, heterocycle, —NR^{x′}R^{y′}, —NR^{x′}C(═O)R^{y′}, —NR^{x′}SO₂R^{y′}, —C(═O)R^{x′}, —C(═O)OR^{x′}, —C(═O)NR^{x′}R^{y′}, —SO_{n′}R^{x′}, and —SO_{n′}NR^{x′}R^{y′}, wherein n′ is 0, 1, or 2, and R^{x′} and R^{y′} are each independently hydrogen, alkyl, or heterocycle;
Y is selected from the group consisting of absent, —C(═O)—, —O—, —OC(═O), —C(═O)O—, —N(R^{b})C(═O)—, —C(═O)N(R^{b})—, —N(R^{b})C(═O)O—, and —OC(═O)N(R^{b})—;
Z¹ is a C₁ to C₆ alkyl that is substituted with three or four R^{x} groups, wherein each R^{x} is independently selected from C₆ to C₁₁ alkyl, C₆ to C₁₁ alkenyl, and C₆ to C₁₁ alkynyl, which C₆ to C₁₁ alkyl, C₆ to C₁₁ alkenyl, and C₆ to C₁₁ alkynyl is optionally substituted with one or more groups independently selected from oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}, wherein n is 0, 1, or 2, and R^{x} and R^{y} are each independently hydrogen, alkyl, or heterocycle, wherein any alkyl and heterocycle of R^{x} and R^{y} may be further substituted with one or more groups independently selected from oxo, halogen, —OH, —CN, alkyl, —OR′, heterocycle, —NR^{x′}R^{y′}, —NR^{x′}C(═O)R^{y′}, —NR^{x′}SO₂R^{y′}, —C(═O)R^{x′}, —C(═O)OR^{x′}, —C(═O)NR^{x′}R^{y′}, —SO_{n′}R^{x′}, and —SO_{n′}NR^{x′}R^{y′}, wherein n′ is 0, 1, or 2, and R^{x′} and R^{y′} are each independently hydrogen, alkyl, or heterocycle;
each R is independently alkyl, alkenyl, or alkynyl, that is optionally substituted with one or more groups independently selected from oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}, wherein n is 0, 1, or 2, and R^{x} and R^{y} are each independently hydrogen, alkyl, or heterocycle, wherein any alkyl and heterocycle of R^{x} and R^{y} may be further substituted with one or more groups independently selected from oxo, halogen, —OH, —CN, alkyl, —OR^{x′}, heterocycle, —NR^{x′}R^{y′}, —NR^{x′}C(═O)R^{y′}, —NR^{x′}SO₂R^{y′}, —C(═O)R^{x′}, —C(═O)OR^{x′}, —C(═O)NR^{x′}R^{y′}, —SO_{n′}R^{x′}, and —SO_{n′}NR^{x′}R^{y′}, wherein n′ is 0, 1, or 2, and R^{x′} and R^{y′} are each independently hydrogen, alkyl, or heterocycle; and
each R^{b} is H or C₁ to C₆alkyl.

In some embodiments, the lipid disclosed in US 2019/0240354 is selected from the group consisting of:

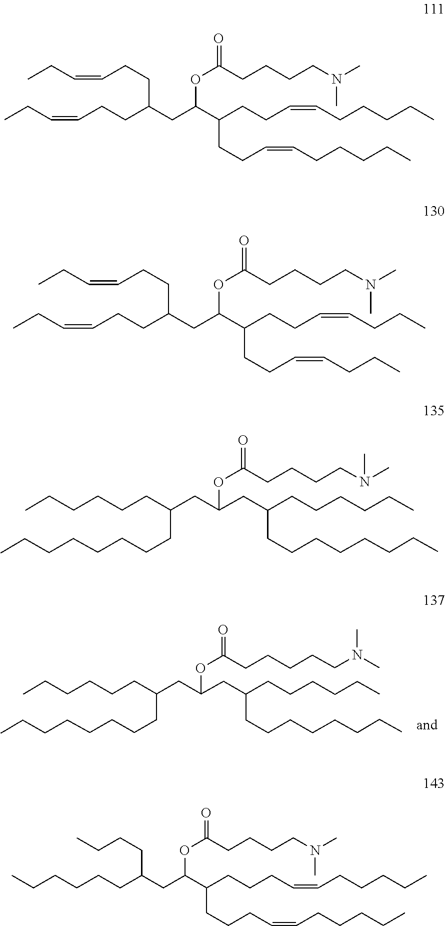

and salts thereof.

In some embodiments, the lipid disclosed in US 2019/0240354 is selected from the group consisting of:

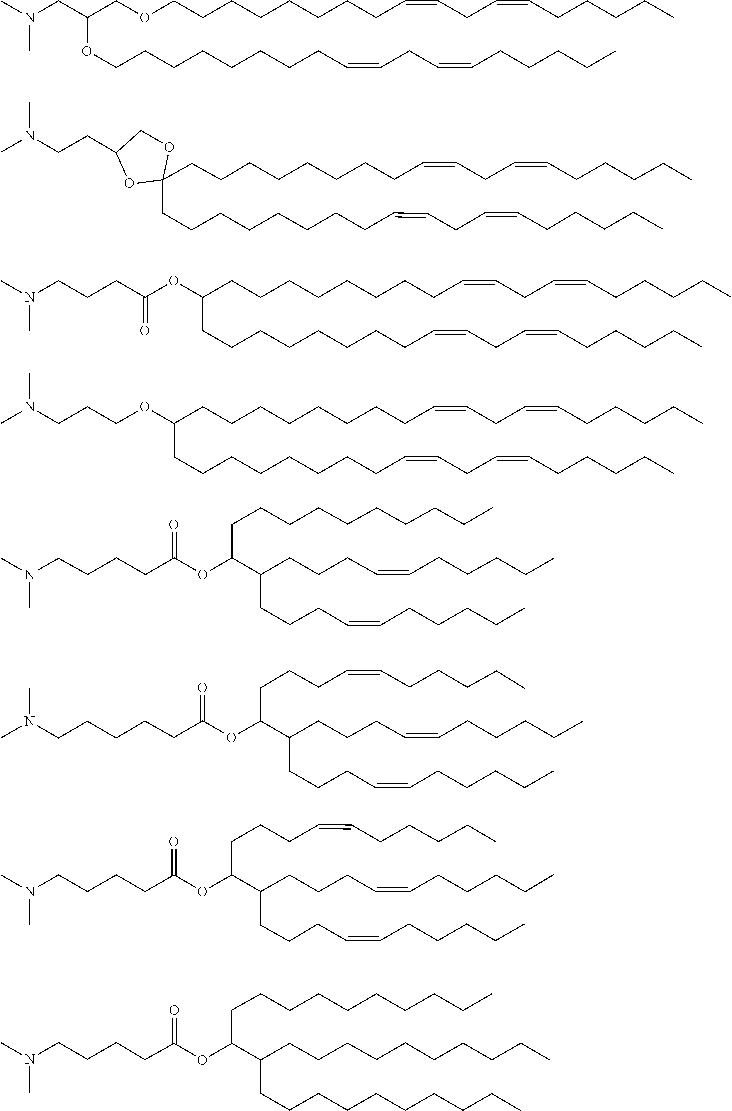

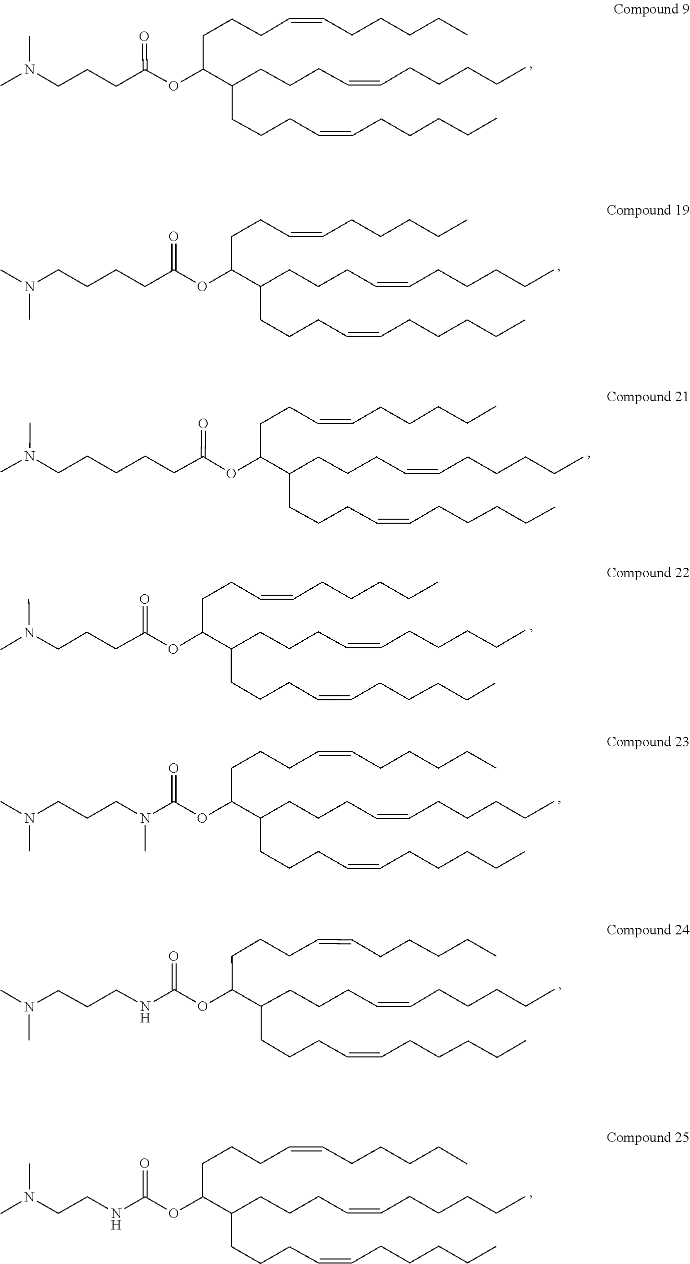

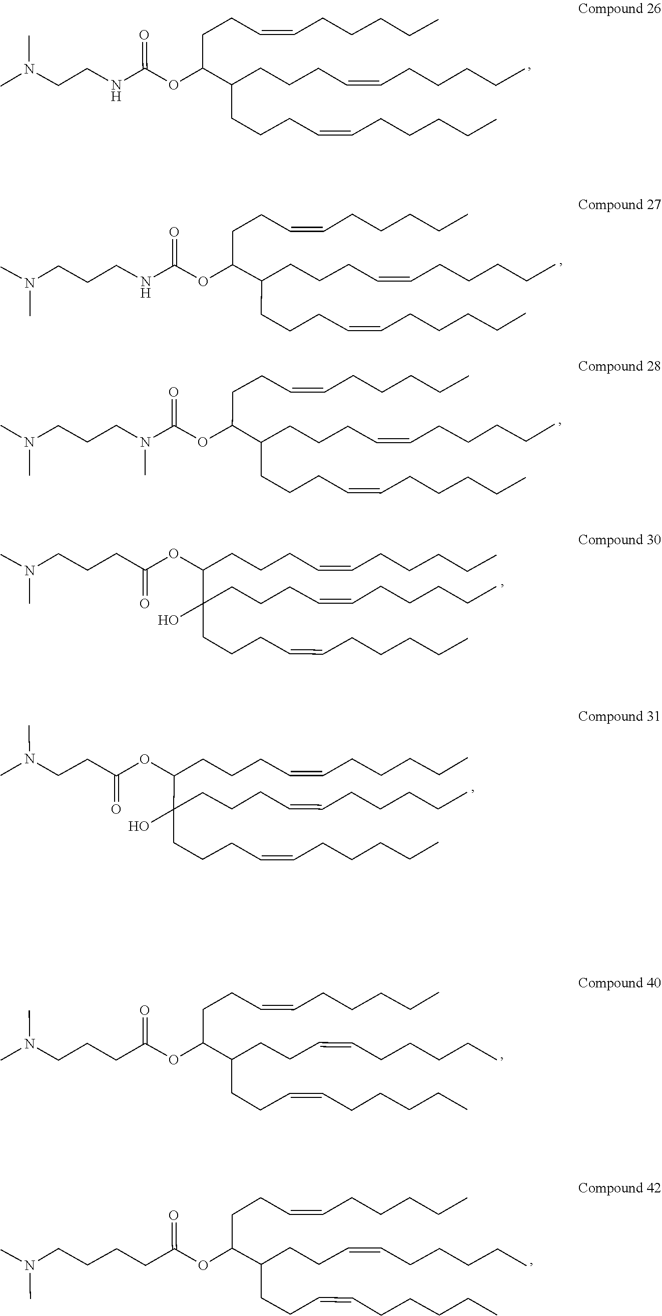

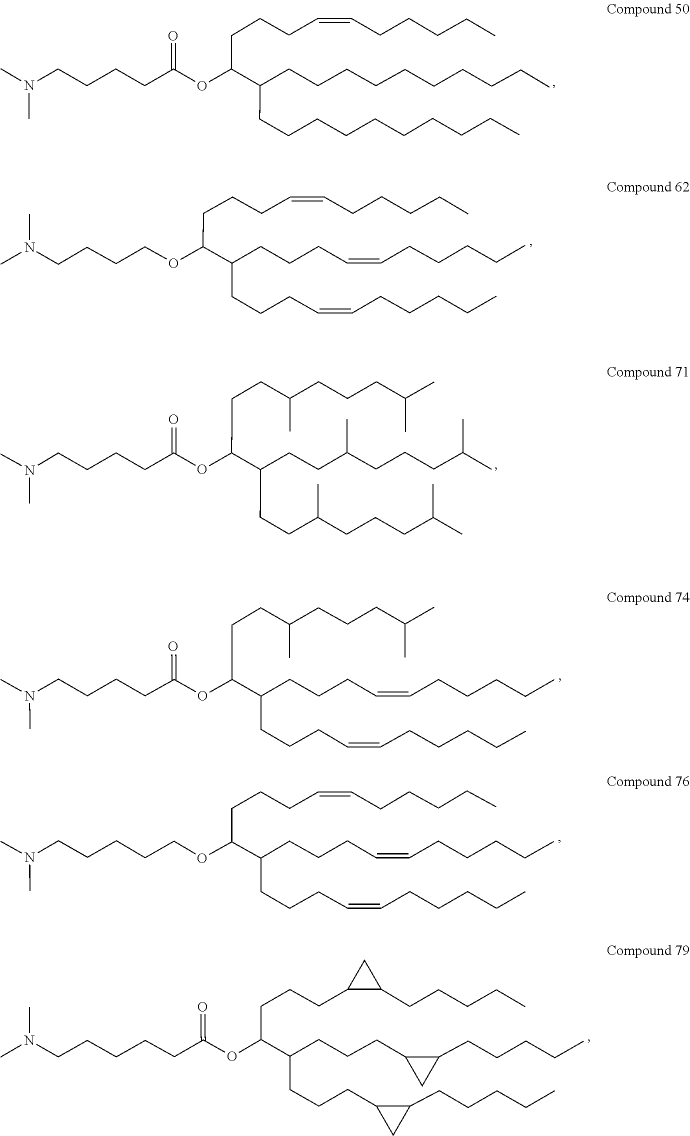

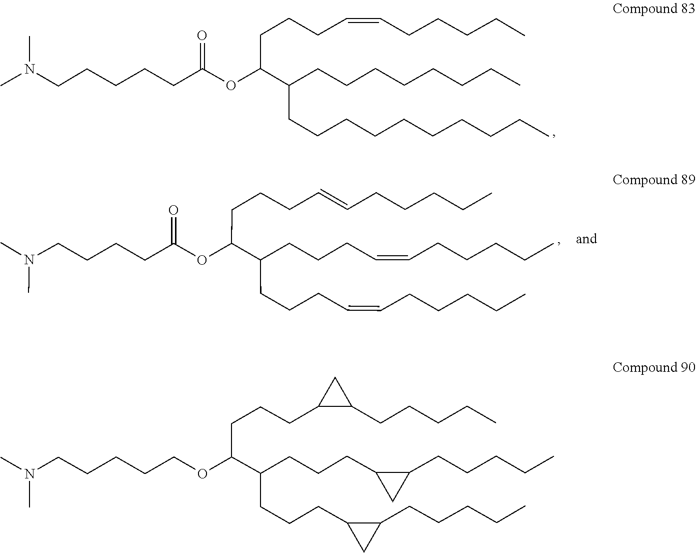

In some embodiments, a LNP described herein comprises a lipid, e.g., an ionizable lipid, disclosed in US 2010/0130588, which is incorporated herein by reference in its entirety.

In some embodiments, the lipids disclosed in US 2010/0130588 are of Formula I:

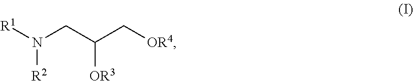

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In some embodiments, R³ and R⁴ are both the same, i.e., R³ and R⁴ are both linoleyl (C₁₈), etc. In some embodiments, R³ and R⁴ are different, i.e., R³ is tetradectrienyl (C₁₄) and R⁴ is linoleyl (C₁₈).

In some embodiments, the lipid of Formula I is 1,2-dilinoleyloxy-N,N-dimethylaminopropane (DLinDMA) or 1,2-dilinolenyloxy-N,N-dimethylaminopropane (DLenDMA).

In some embodiments, the lipids disclosed in US 2010/0130588 are of Formula II:

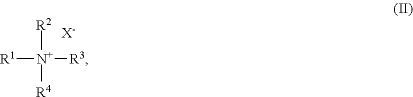

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation.

In some embodiments, a LNP described herein comprises a lipid, e.g., an ionizable lipid, disclosed in US 2021/0087135, which is incorporated herein by reference in its entirety.

In some embodiments, the lipids disclosed in US 2021/0087135 are of Formula (A):

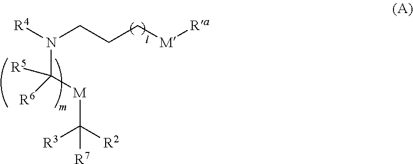

or its N-oxide, or a salt or isomer thereof,
wherein R′^{a} is R′^{branched} or R′^{cyclic}; wherein
R′^{branched} is:

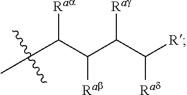

R′^{cyclic} is:

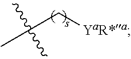

wherein:

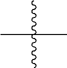

denotes a point of attachment;
wherein R^{aα} is H, and R^{aβ}, R^{aγ}, and R^{aδ} are each independently selected from the group consisting of H, C₂₋₁₂ alkyl, and C₂₋₁₂ alkenyl, wherein at least one of R^{aβ}, R^{aγ}, and R^{aδ} is selected from the group consisting of C₂₋₁₂ alkyl and C₂₋₁₂ alkenyl;
R² and R³ are each C₁₋₁₄ alkyl;
R⁴ is selected from the group consisting of —(CH₂)₂OH, —(CH₂)₃OH, —(CH₂)₄OH, —(CH₂)₅OH and

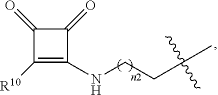

wherein:

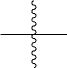

denotes a point of attachment;
R¹⁰ is N(R)2; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
R⁷ is H;
M and M′ are each independently selected from the group consisting of —C(O)O— and —OC(O)—;
R′ is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl;
1 is selected from the group consisting of 1, 2, 3, 4, and 5;
s is 2 or 3; and
m is selected from the group consisting of 5, 6, 7, 8, 9, 10, 11, 12, and 13.

In some embodiments, the lipid disclosed in US 2021/0087135 is:

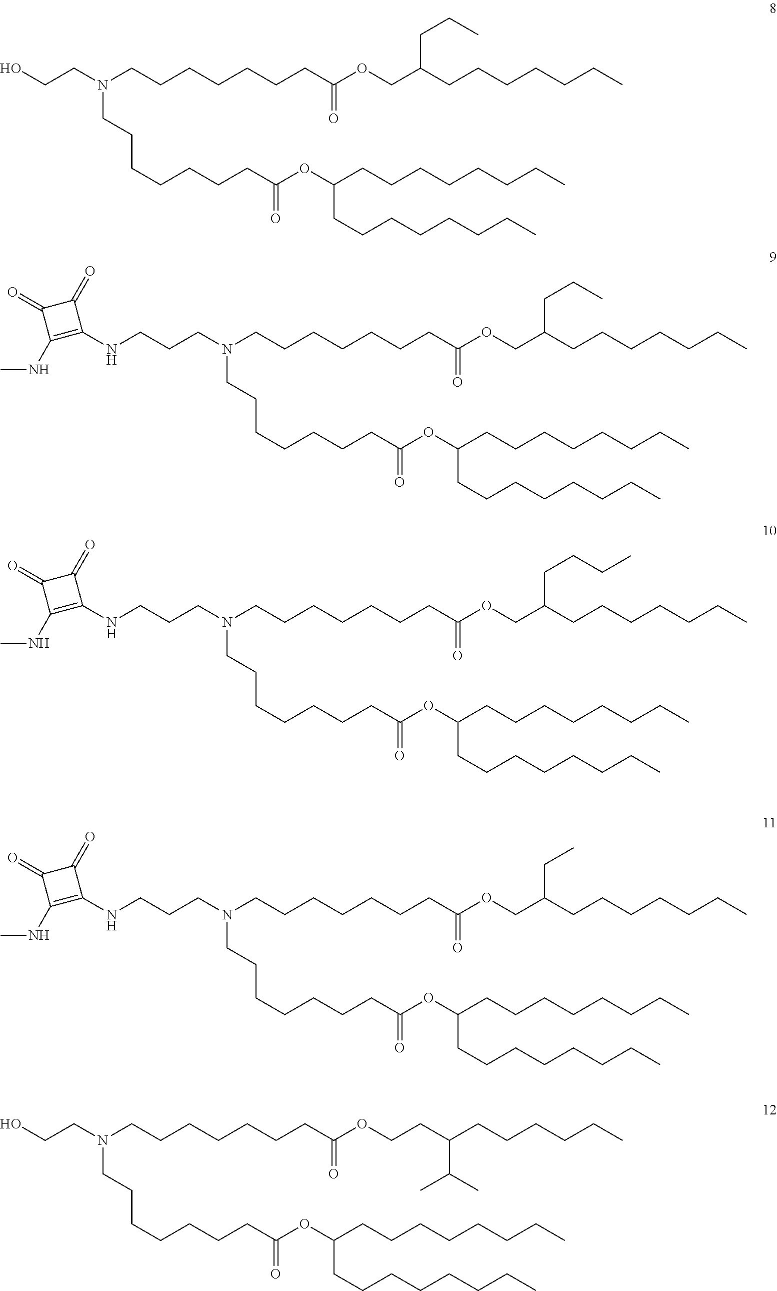

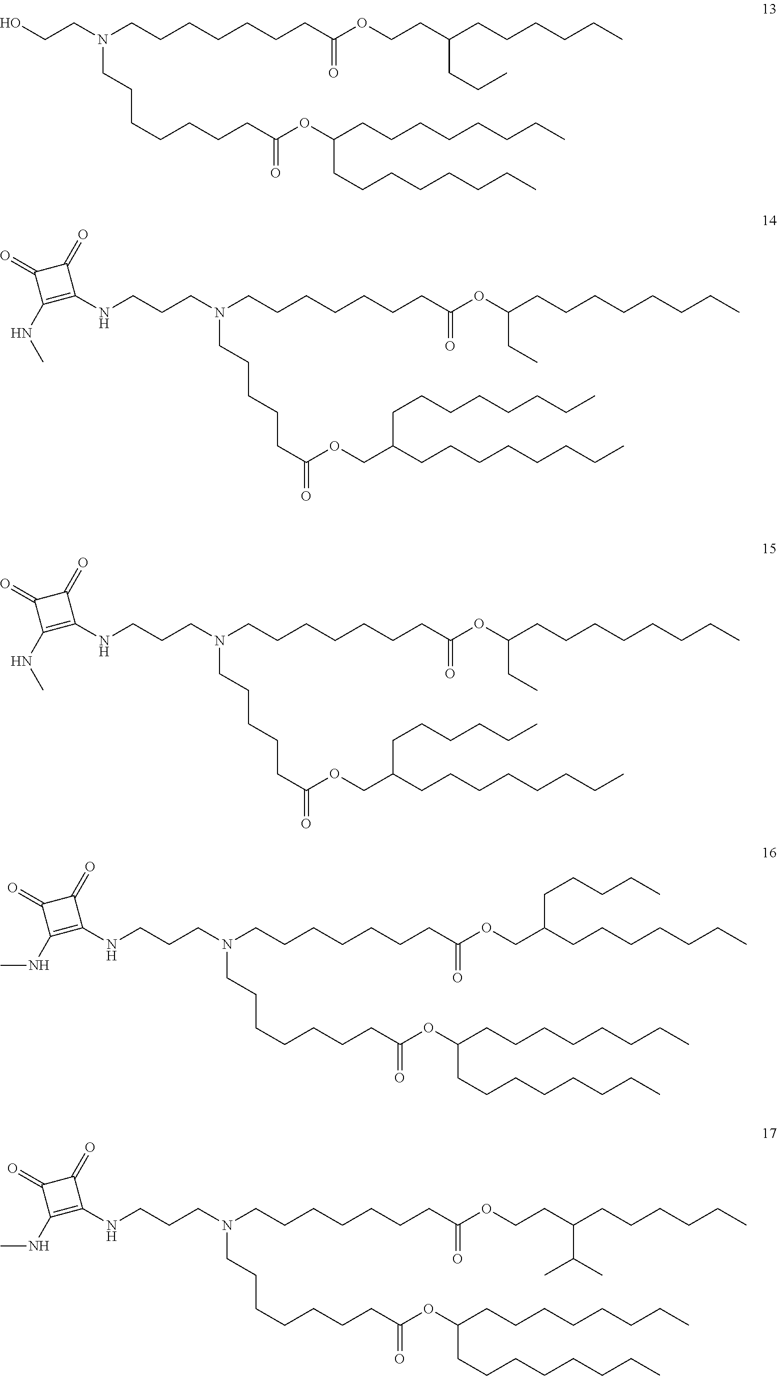

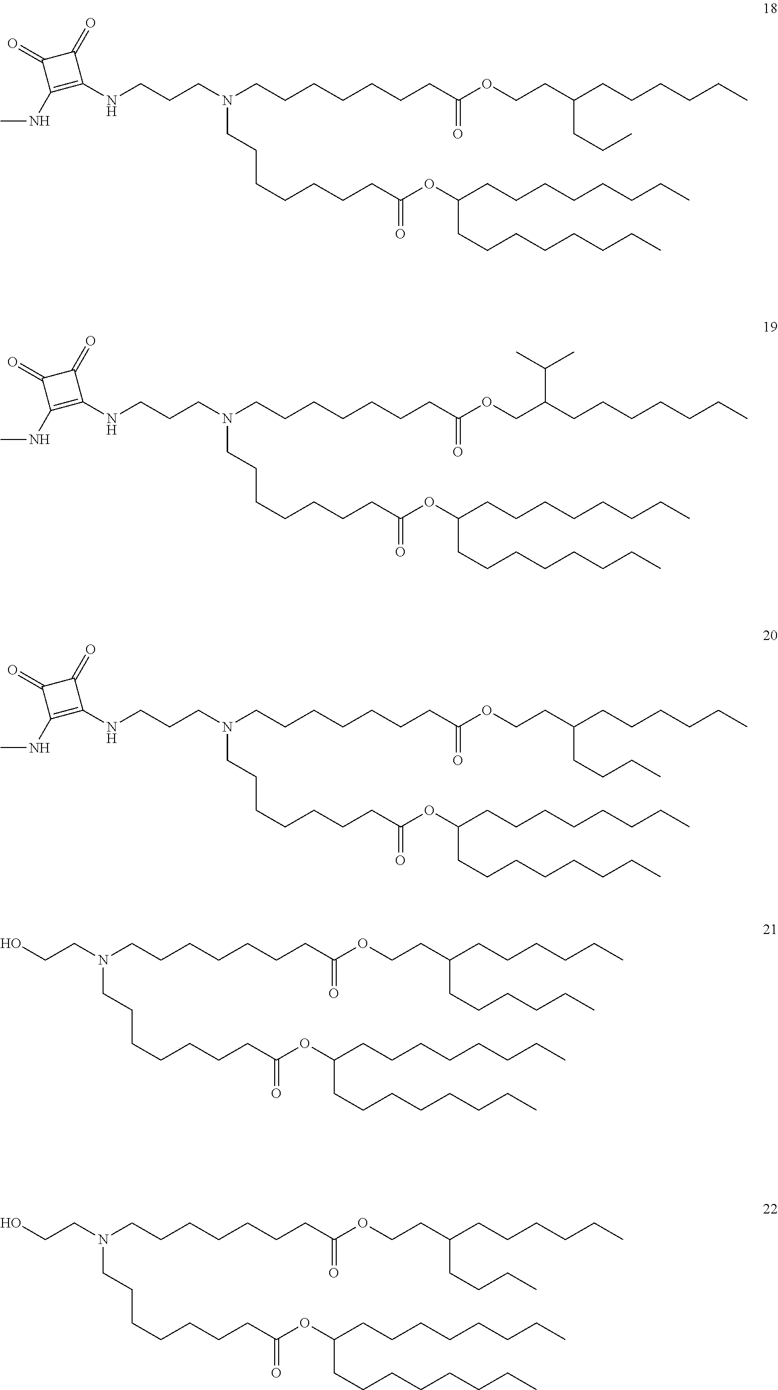

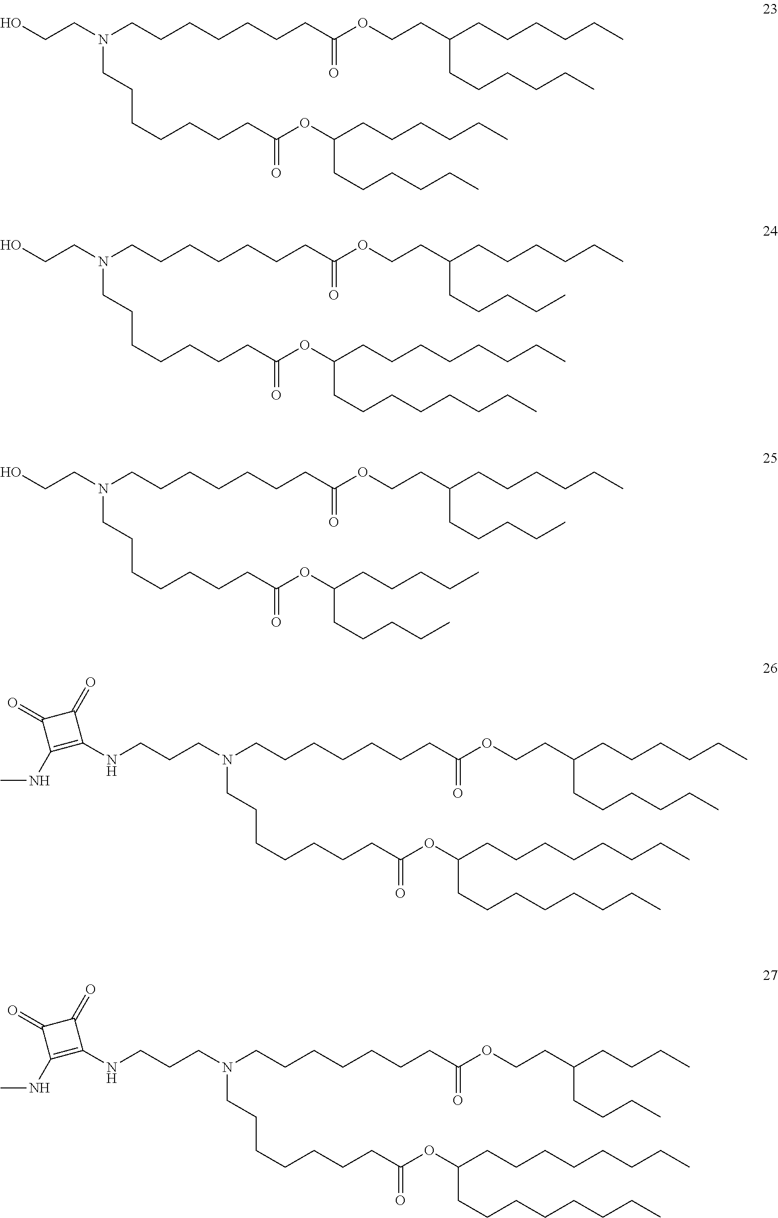

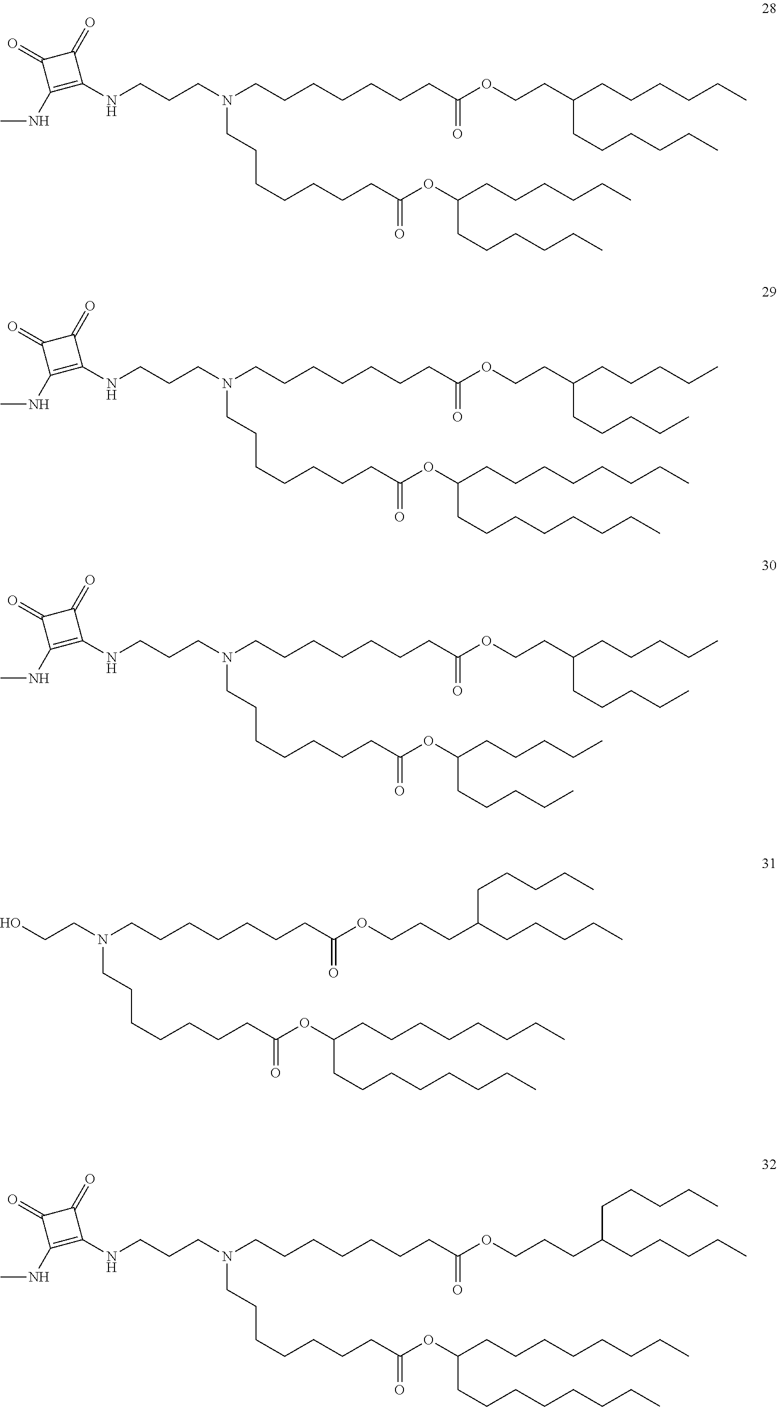

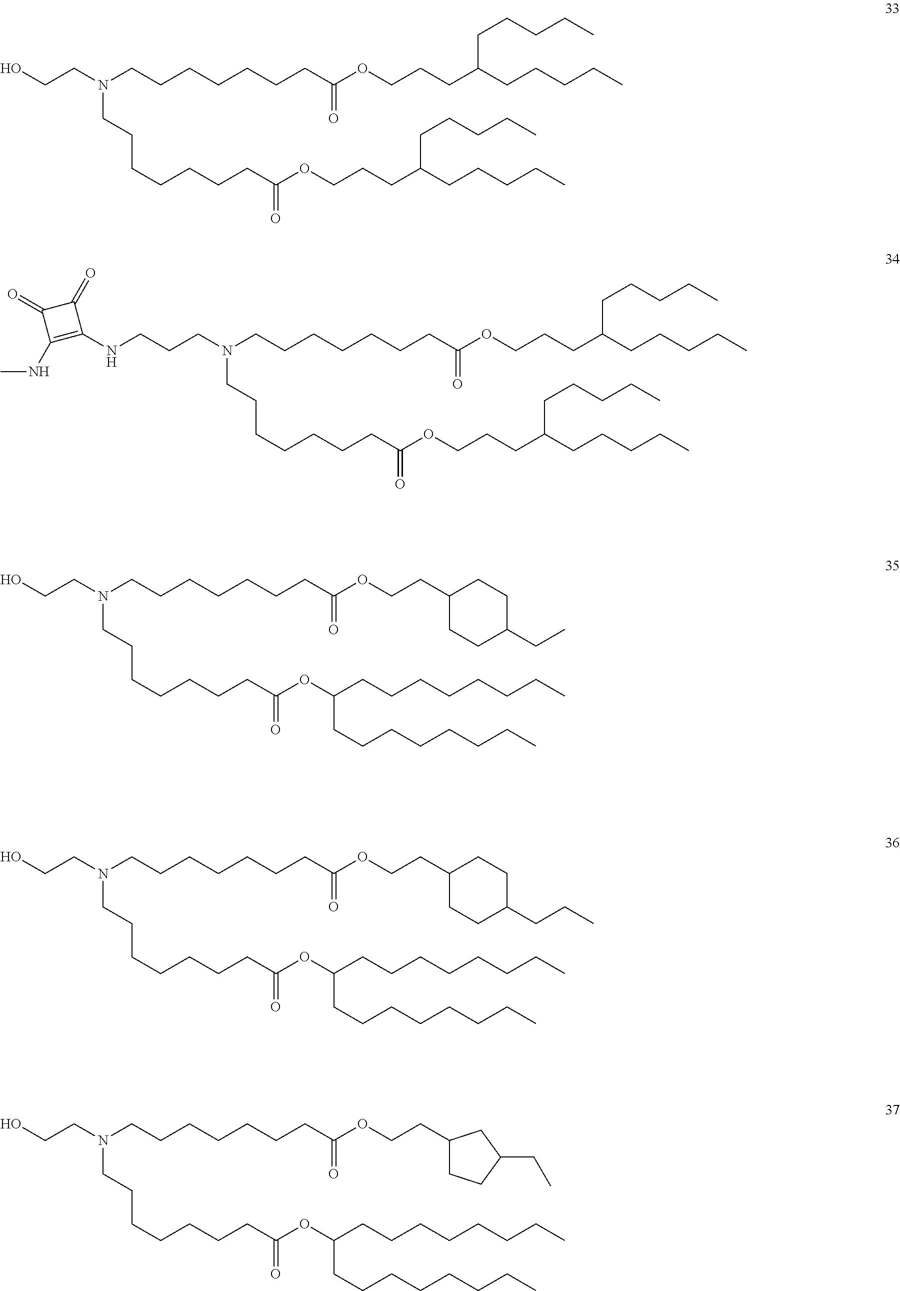

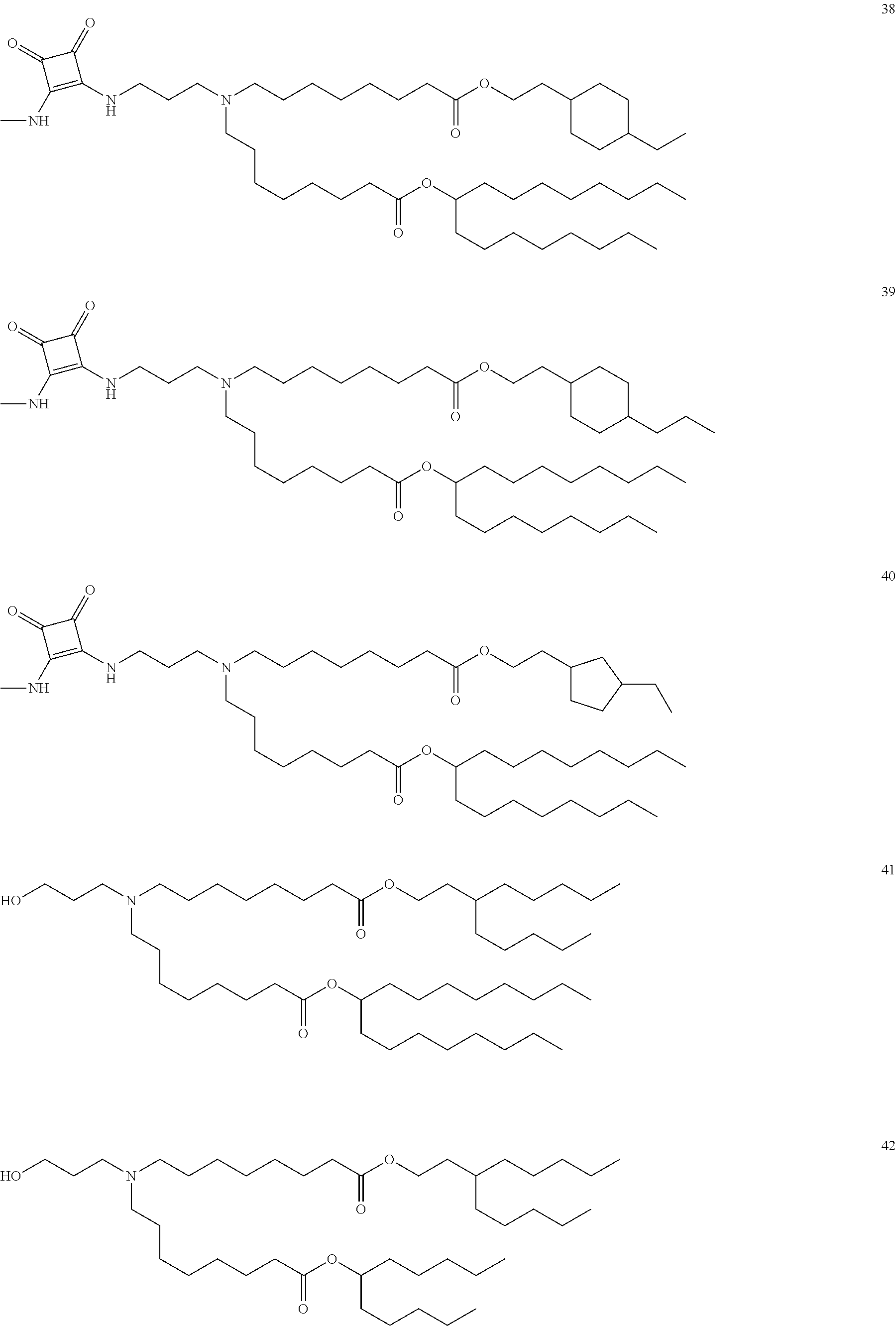

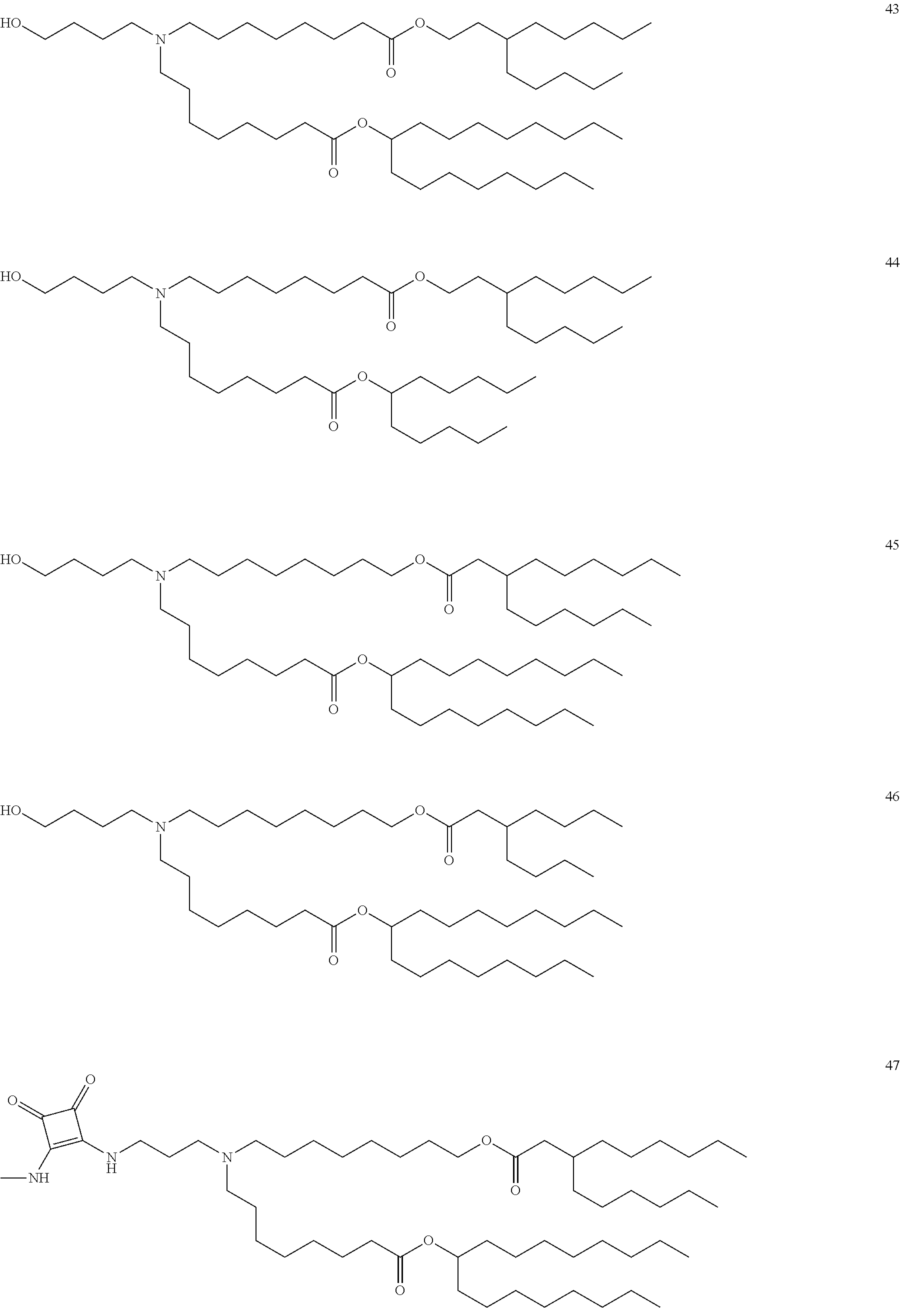

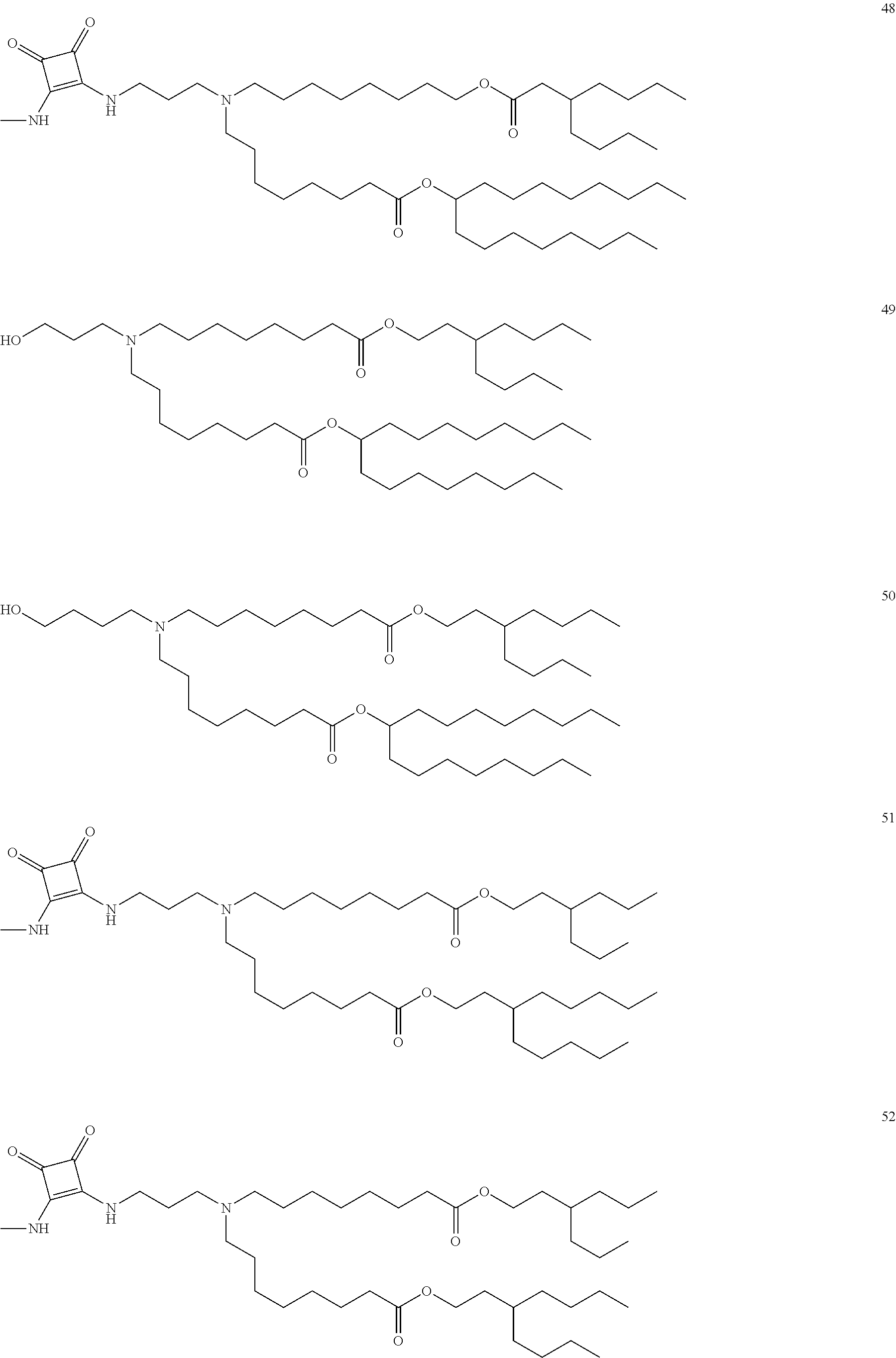

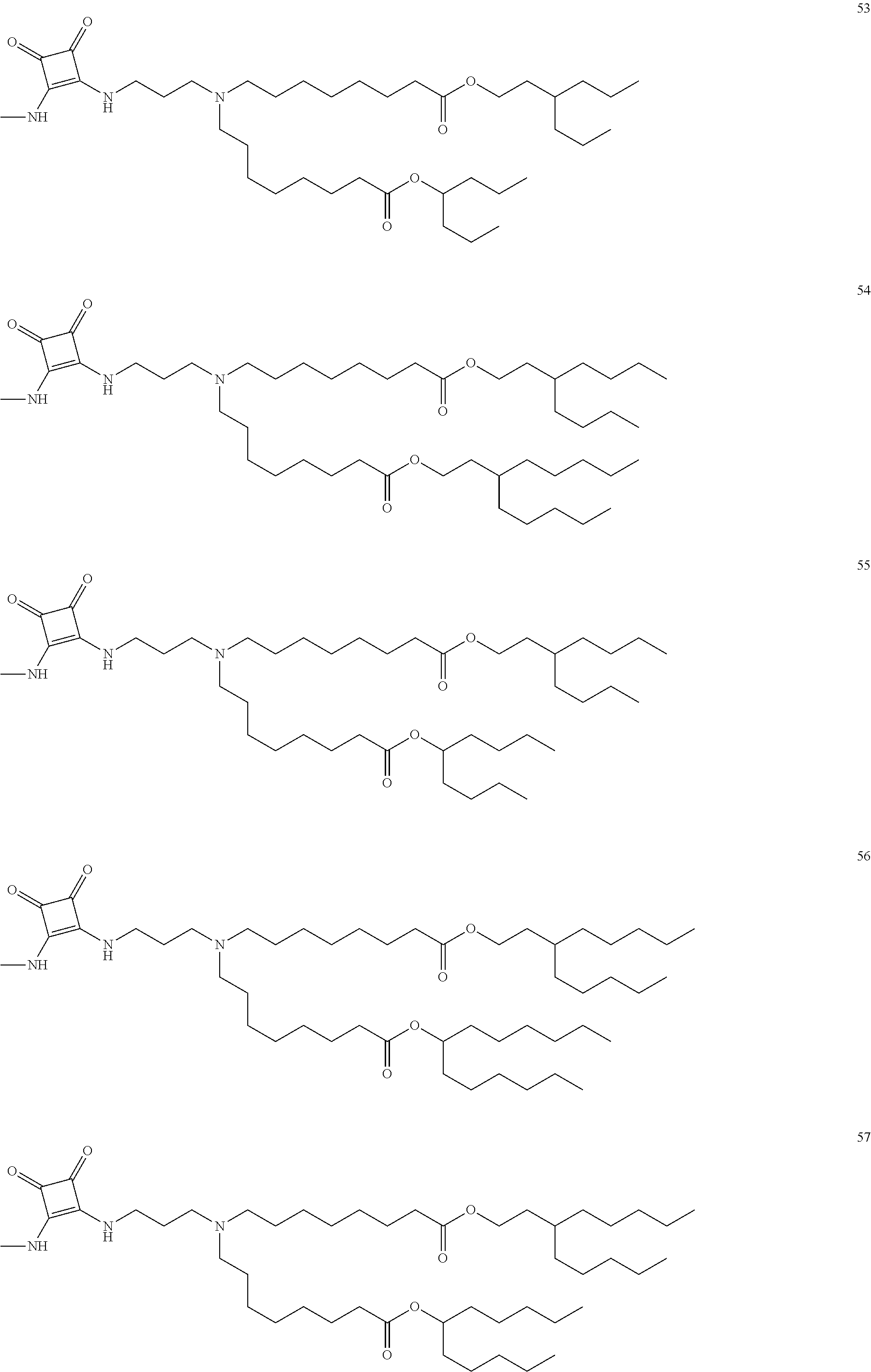

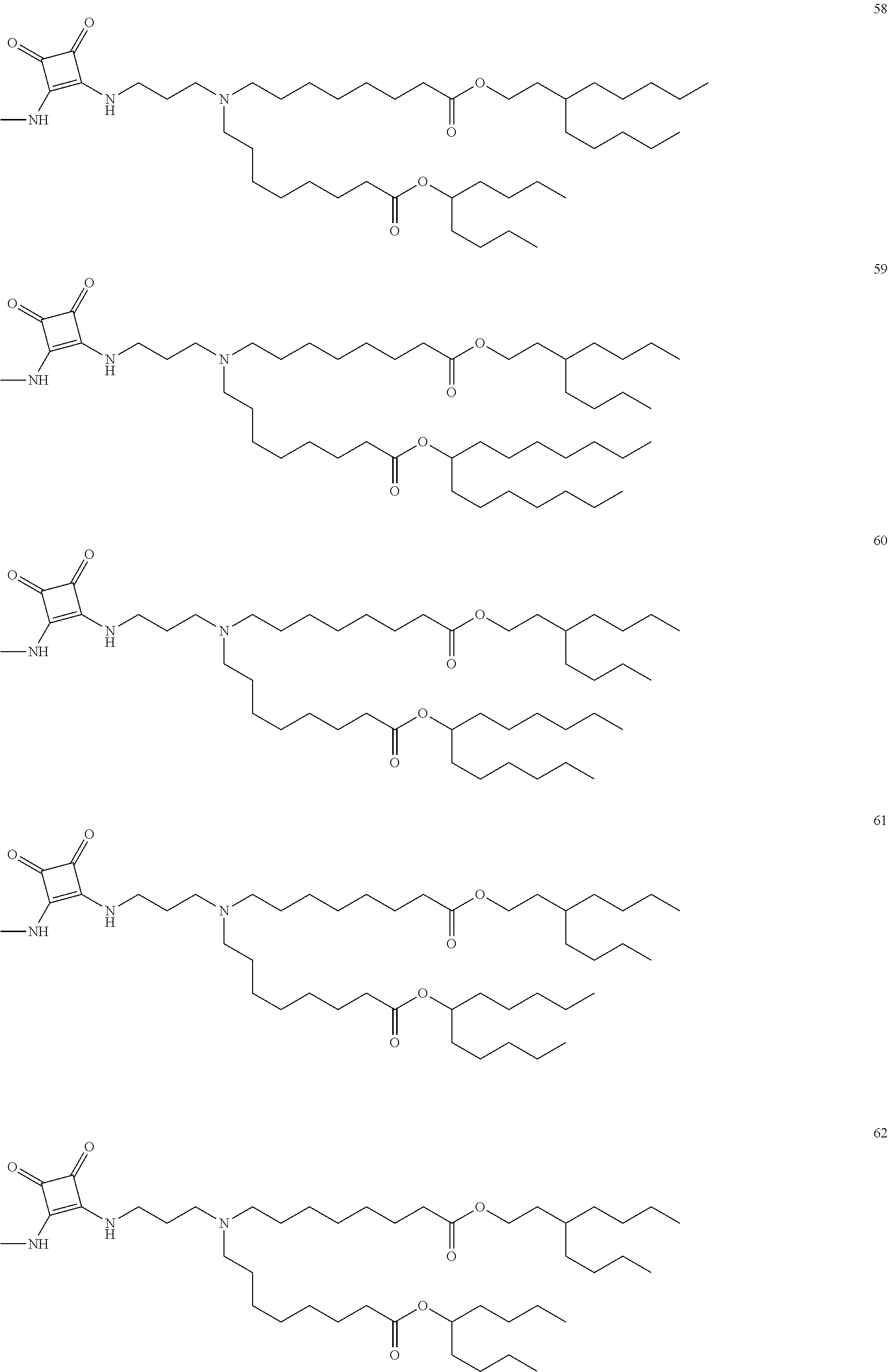

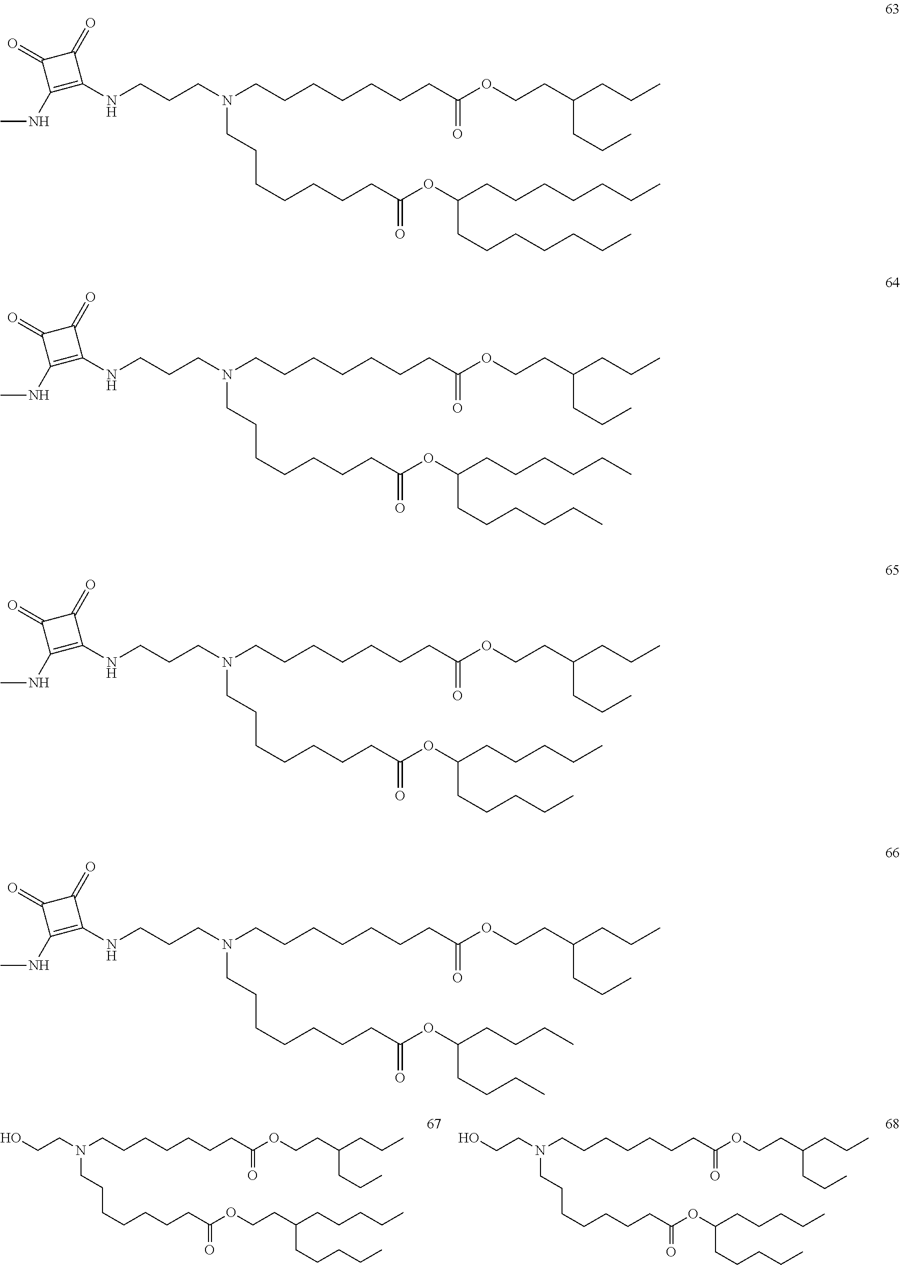

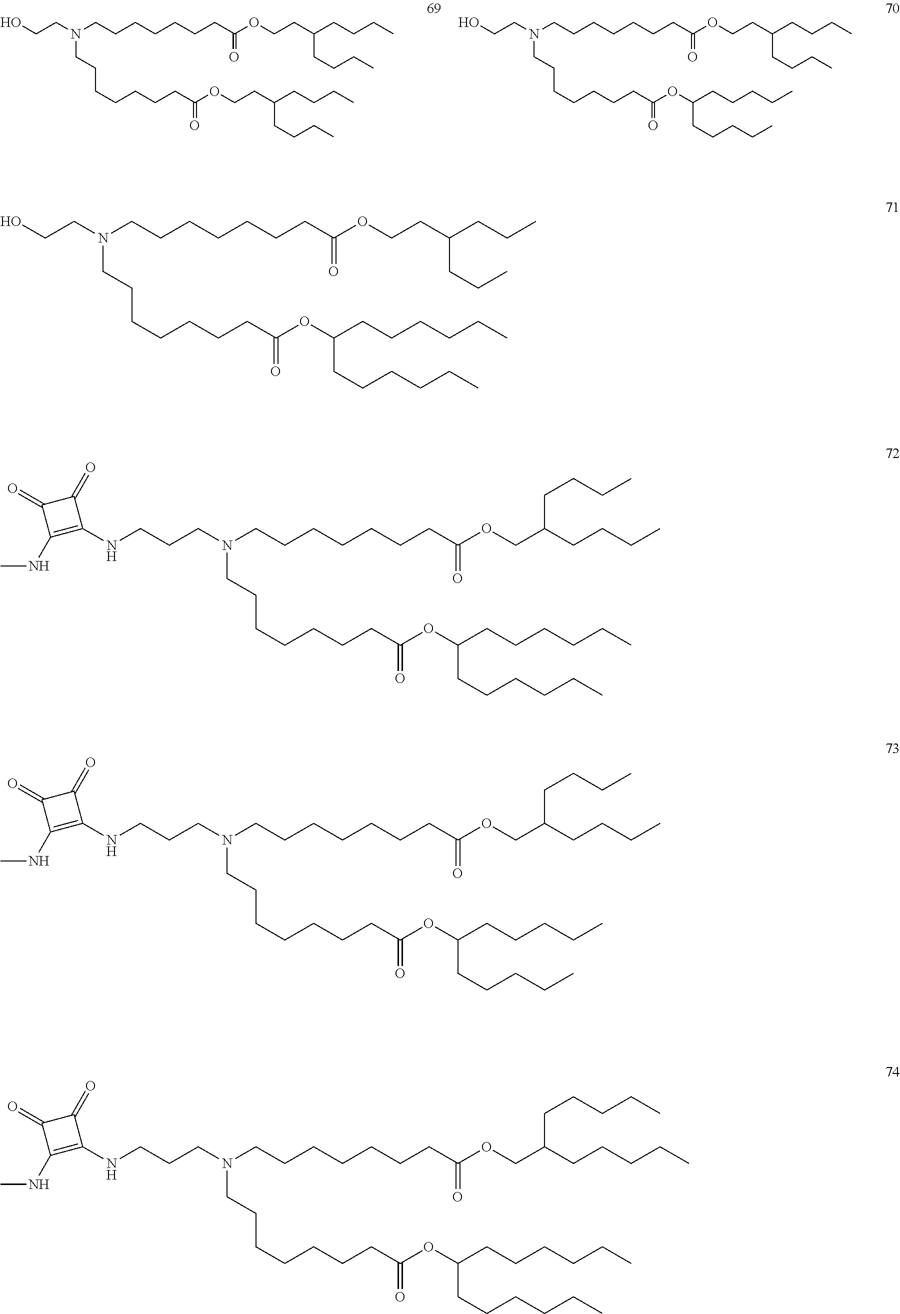

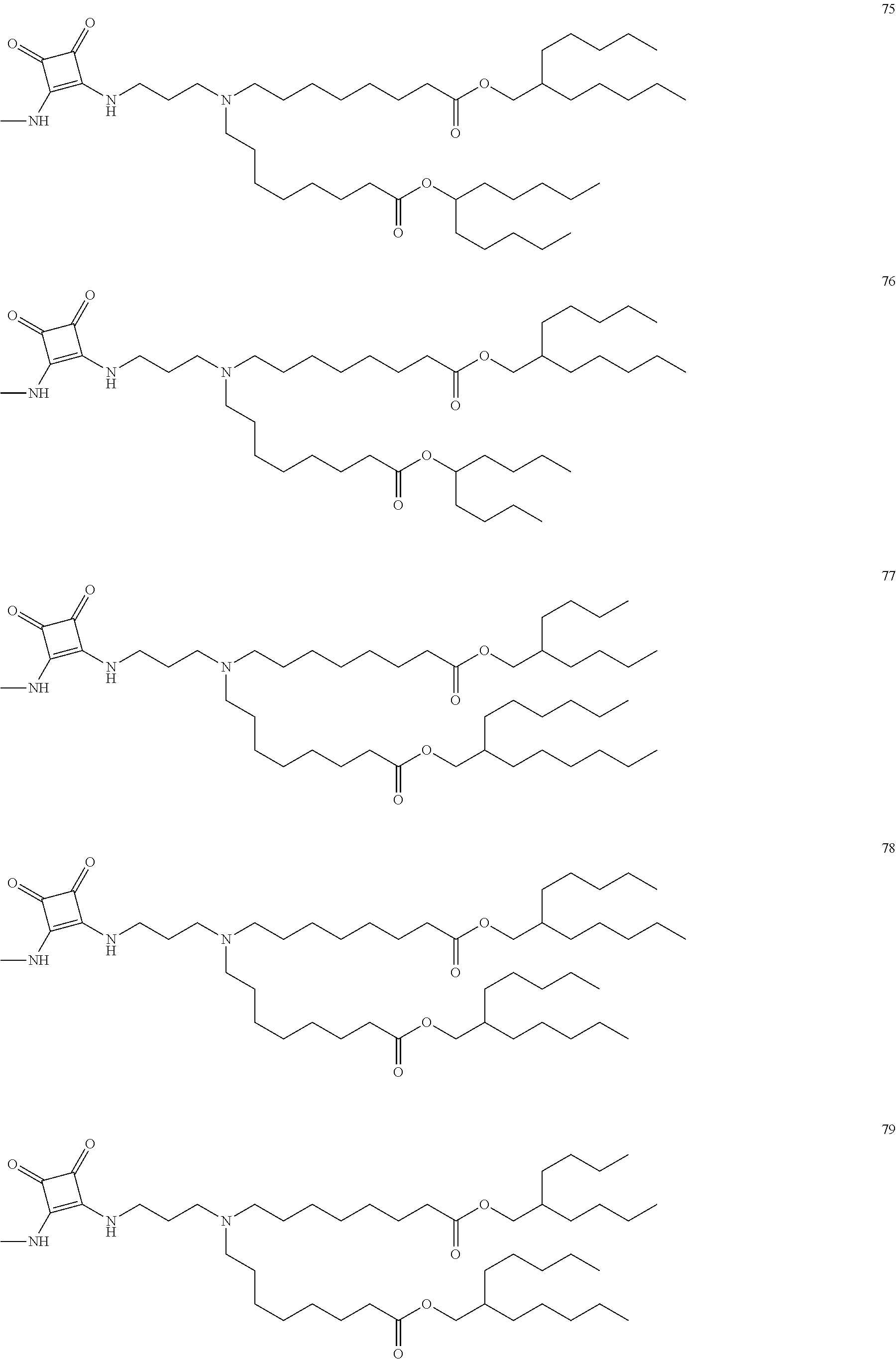

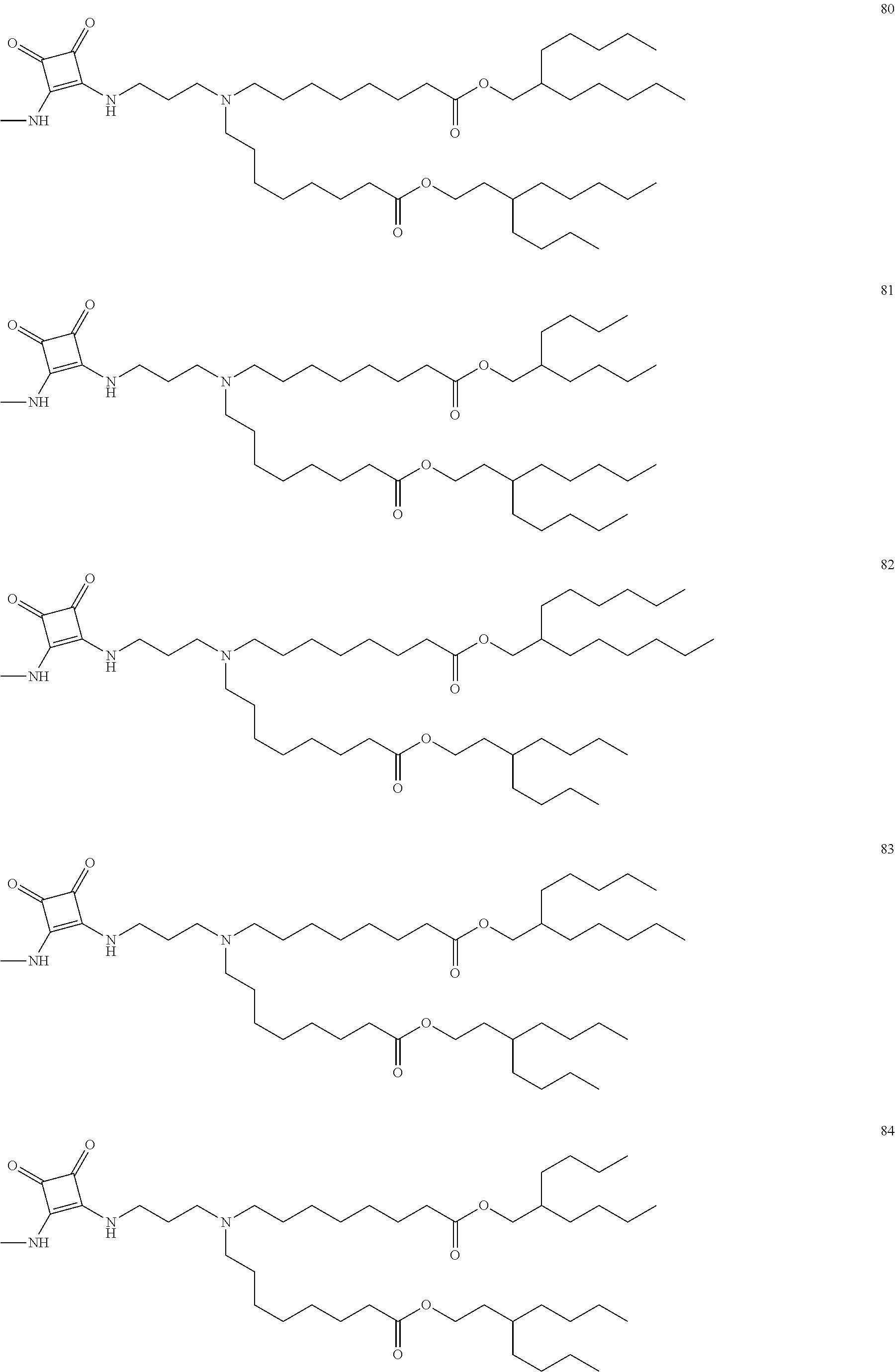

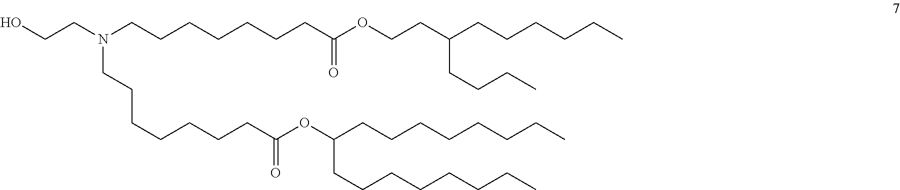 [SMILES: CCCCCCCCC(CCCCCCCC)OC(=O)CCCCCCCN(CCO)CCCCCCCC(=O)OCCC(CCCC)CCCCCC]

In some embodiments, a LNP described herein comprises a lipid, e.g., an ionizable lipid, disclosed in US 2021/0128488, which is incorporated herein by reference in its entirety

In some embodiments, the lipids disclosed in US 2021/0128488 are of structure (I):

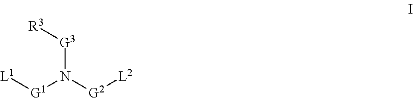

or a pharmaceutically acceptable salt, prodrug or stereoisomer thereof, wherein:
L¹ is —O(C═O)R′, —(C═O)OR¹, —C(═O)R¹, —OR¹, —S(O)ₓR¹, —S—SR¹, —C′(′O)SR′, —SC(═O)R′, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and wherein each alkyl, alkenyl, alkylene, alkenylene, cycloalkylene, cycloalkenylene, aryl and aralkyl is independently substituted or unsubstituted unless otherwise specified.

In some embodiments, a LNP described herein comprises a lipid, e.g., an ionizable lipid, disclosed in US 2020/0121809, which is incorporated herein by reference in its entirety.

In some embodiments the lipids disclosed in US 2020/0121809 have a structure of Formula II:

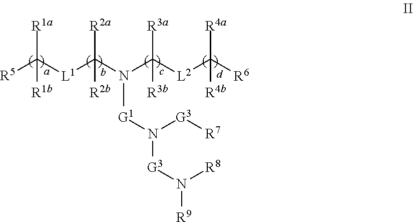

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{1b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments, the lipids disclosed in US 2020/0121809 have a structure of Formula III:

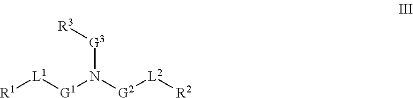

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some embodiments, the lipids disclosed in US 2020/0121809 have a structure of Formula (IV):

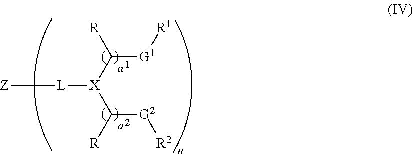

or a pharmaceutically acceptable salt, prodrug or stereoisomer thereof, wherein:

one of G¹ or G² is, at each occurrence, —O(C═O)—, —(C═O)O—, —C(═O)—, —O—, —S(O)_{y}—, —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O)_{y}—, —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, —O(C═O)—, wherein — represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

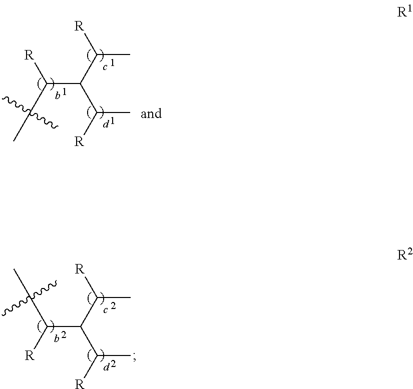

a¹ and a² are, at each occurrence, independently an integer from 3 to 12;
b¹ and b² are, at each occurrence, independently 0 or 1;
c¹ and c² are, at each occurrence, independently an integer from 5 to 10;
d¹ and d² are, at each occurrence, independently an integer from 5 to 10;
y is, at each occurrence, independently an integer from 0 to 2; and
n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments of Formula (IV), the compound has one of the following structures:

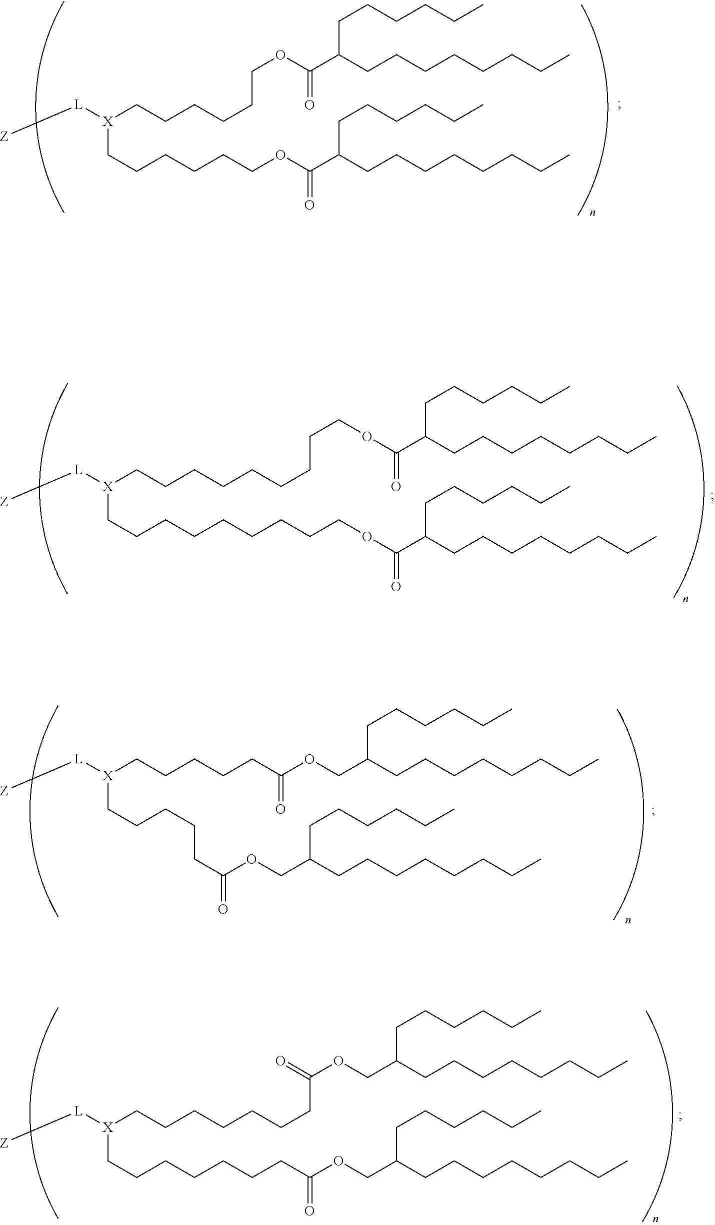

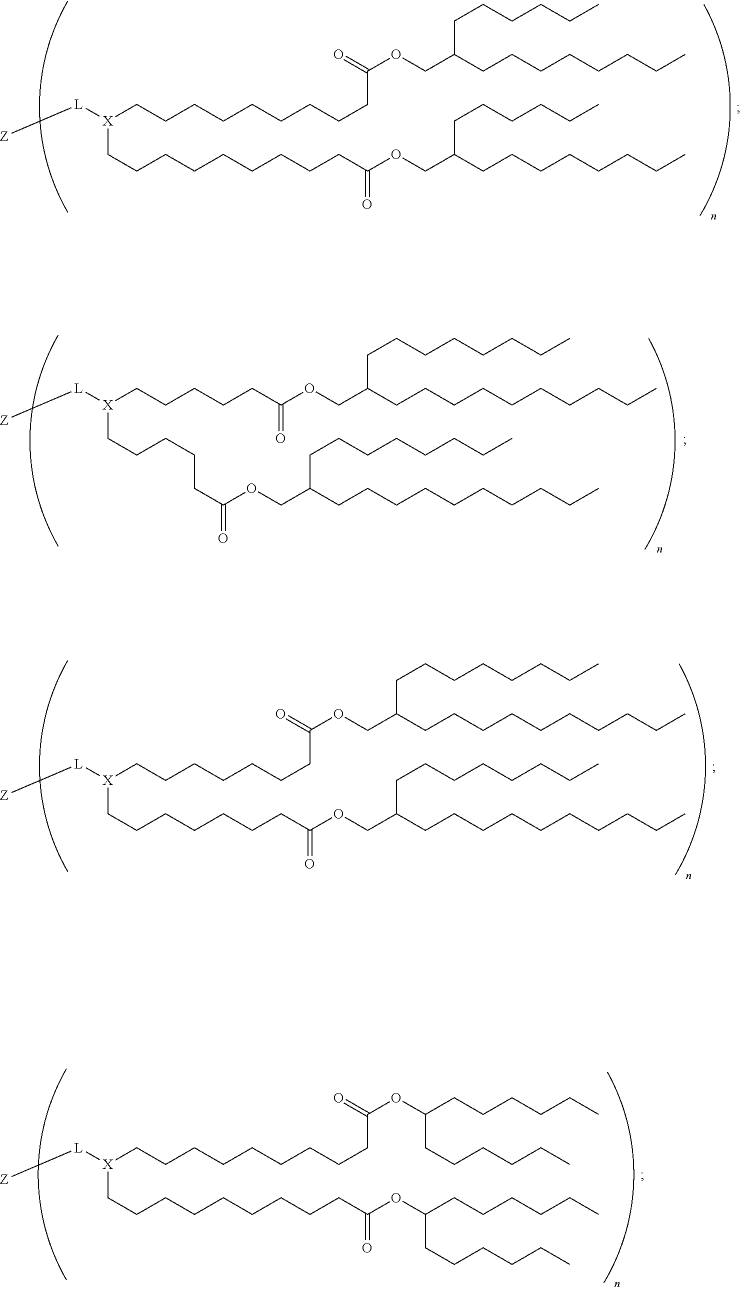

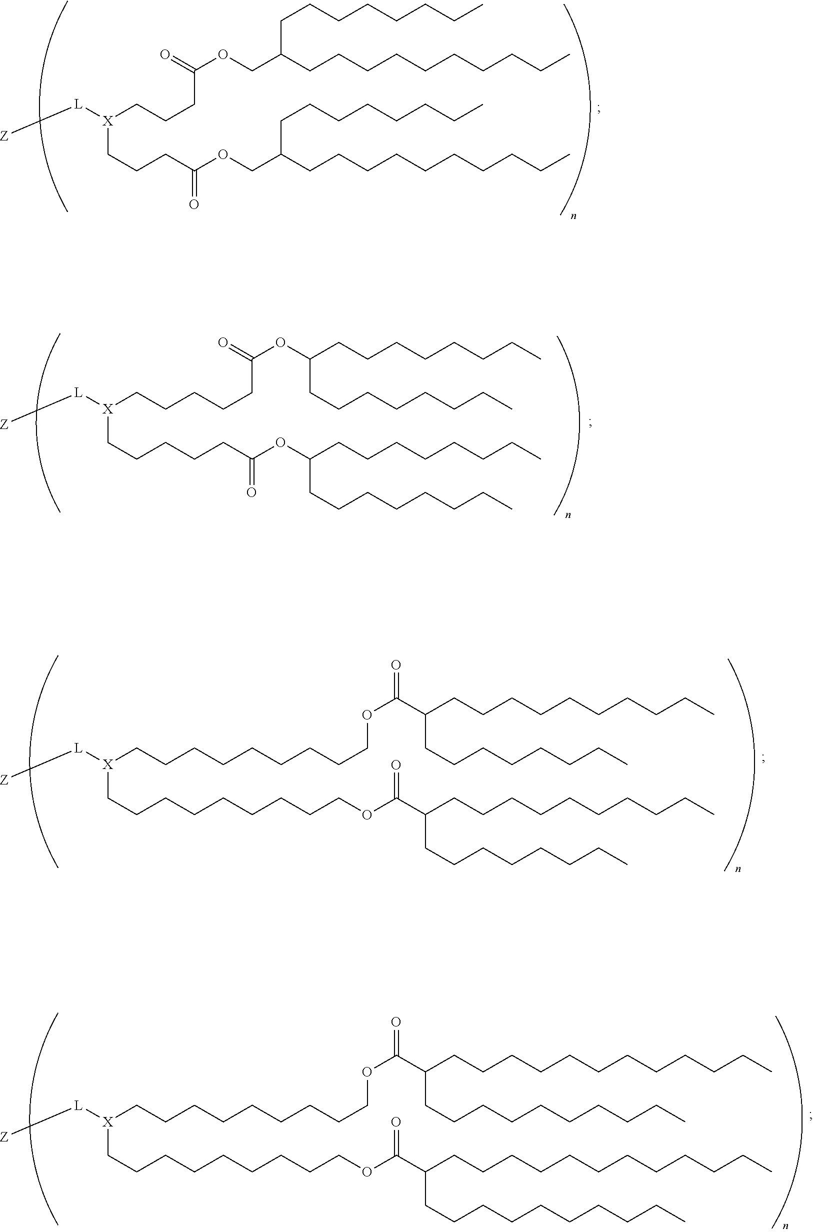

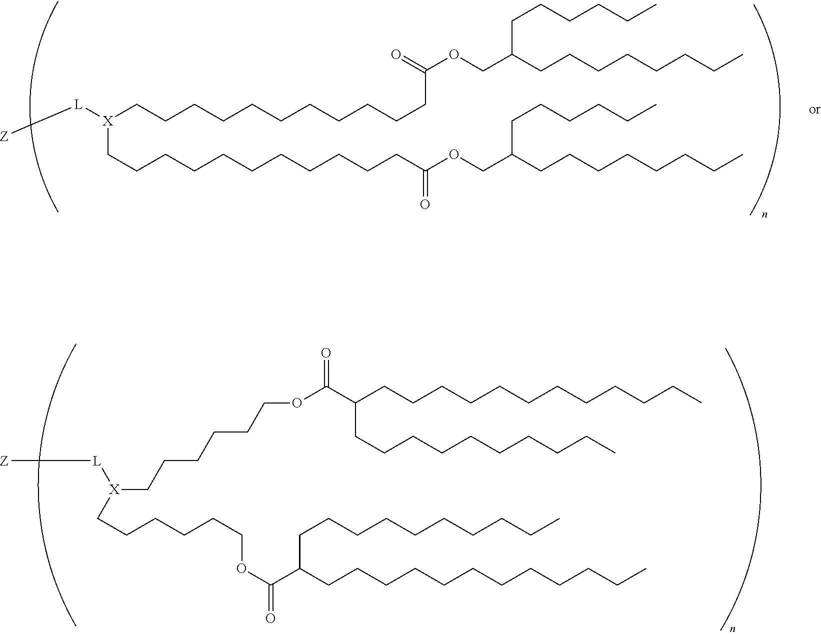

In some embodiments, a LNP described herein comprises a lipid, e.g., an ionizable lipid, disclosed in US 2013/0108685, which is incorporated herein by reference in its entirety.

In some embodiments, the lipids disclosed in US 2013/0108685 are represented by the following formula (I):

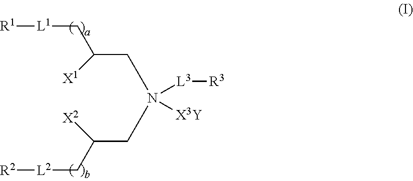

(wherein:
R¹ and R² are, the same or different, each linear or branched alkyl, alkenyl or alkynyl having 12 to 24 carbon atoms, or R¹ and R² are combined together to form dialkylmethylene, dialkenylmethylene, dialkynylmethylene or alkylalkenylmethylene,
X¹ and X³ are hydrogen atoms, or are combined together to form a single bond or alkylene,
X³ is absent or represents alkyl having 1 to 6 carbon atoms, or alkenyl having 3 to 6 carbon atoms,
when X³ is absent,
Y is absent, a and b are 0, L³ is a single bond, R³ is alkyl having 1 to 6 carbon atoms, alkenyl having 3 to 6 carbon atoms, pyrrolidin-3-yl, piperidin-3-yl, piperidin-4-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, and L¹ and L² are —O—,
Y is absent, a and b are, the same or different, 0 to 3, and are not 0 at the same time, L³ is a single bond, R³ is alkyl having 1 to 6 carbon atoms, alkenyl having 3 to 6 carbon atoms, pyrrolidin-3-yl, piperidin-3-yl, piperidin-4-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, L¹ and L² are, the same or different, —O—, —CO—O— or —O—CO—,
Y is absent, a and b are, the same or different, 0 to 3, L³ is a single bond, R³ is a hydrogen atom, and L¹ and L² are, the same or different, —O—, —CO—O— or —O—CO—, or
Y is absent, a and b are, the same or different, 0 to 3, L³ is —CO— or —CO—O—, R³ is pyrrolidin-2-yl, pyrrolidin-3-yl, piperidin-2-yl, piperidin-3-yl, piperidin-4-yl, morpholin-2-yl, morpholin-3-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, wherein at least one of the substituents is amino, monoalkylamino, dialkylamino, trialkylammonio, pyrrolidinyl, piperidyl or morpholinyl, and L¹ and L² are, the same or different, —O—, —CO—O— or —O—CO—, and
when X³ is alkyl having 1 to 6 carbon atoms or alkenyl having 3 to 6 carbon atoms,
Y is a pharmaceutically acceptable anion, a and b are, the same or different, 0 to 3, L³ is a single bond, R³ is alkyl having 1 to 6 carbon atoms, alkenyl having 3 to 6 carbon atoms, pyrrolidin-2-yl, pyrrolidin-3-yl, piperidin-2-yl, piperidin-3-yl, piperidin-4-yl, morpholin-2-yl, morpholin-3-yl, or alkyl having 1 to 6 carbon atoms or alkenyl having 3 to 6 carbon atoms substituted with 1 to 3 substituent(s), which is(are), the same or different, amino, monoalkylamino, dialkylamino, trialkylammonio, hydroxy, alkoxy, carbamoyl, monoalkylcarbamoyl, dialkylcarbamoyl, pyrrolidinyl, piperidyl or morpholinyl, L¹ and L² are, the same or different, —O—, —CO—O— or —O—CO—).

In some embodiments, a LNP described herein comprises a lipid, e.g., an ionizable lipid, disclosed in US 2013/0195920, which is incorporated herein by reference in its entirety.

In some embodiments, the lipids disclosed in US 2013/0195920 are of formula (I), which has a branched alkyl at the alpha position adjacent to the biodegradable group (between the biodegradable group and the tertiary carbon):

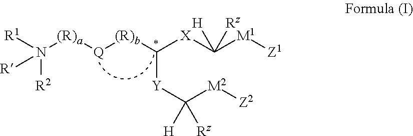

or a salt thereof (e.g., a pharmaceutically acceptable salt thereof), wherein
R′ is absent, hydrogen, or alkyl (e.g., C₁-C₄ alkyl);
with respect to R¹ and R²,
(i) R¹ and R² are each, independently, optionally substituted alkyl, alkenyl, alkynyl, cycloalkylalkyl, heterocycle, or R¹⁰;
(ii) R¹ and R², together with the nitrogen atom to which they are attached, form an optionally substituted heterocylic ring; or
(iii) one of R¹ and R² is optionally substituted alkyl, alkenyl, alkynyl, cycloalkyl, cycloalkylalkyl, or heterocycle, and the other forms a 4-10 member heterocyclic ring or heteroaryl (e.g., a 6-member ring) with (a) the adjacent nitrogen atom and (b) the (R)ₐ group adjacent to the nitrogen atom;
each occurrence of R is, independently, —(CR³R⁴)—;
each occurrence of R³ and R⁴ are, independently H, halogen, OH, alkyl, alkoxy, —NH₂, R¹⁰, alkylamino, or dialkylamino (In some embodiments, each occurrence of R³ and R⁴ are, independently H or C₁-C₄ alkyl);
each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups (preferably at most one R¹⁰ group);
the dashed line to Q is absent or a bond;
when the dashed line to Q is absent then Q is absent or is —O—, —NH—, —S—, —C(O)—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—; or
when the dashed line to Q is a bond then (i) b is 0 and (ii) Q and the tertiary carbon adjacent to it (C*) form a substituted or unsubstituted, mono- or bi-cyclic heterocyclic group having from 5 to 10 ring atoms (e.g., the heteroatoms in the heterocyclic group are selected from O and S, preferably 0);
each occurrence of R⁵ is, independently, H or alkyl (e.g. C₁-C₄ alkyl);
X and Y are each, independently, alkylene or alkenylene (e.g., C₄ to C₂₀ alkylene or C₄ to C₂₀ alkenylene);
M¹ and M² are each, independently, a biodegradable group (e.g., —OC(O)—, —C(O)O—, —SC(O)—, —C(O)S—, —OC(S)—, —C(S)O, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

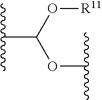

(wherein R¹¹ is a C₂-C₈ alkyl or alkenyl));
each occurrence of R^{z} is, independently, C₁-C₈ alkyl (e.g., methyl, ethyl, isopropyl, n-butyl, n-pentyl, or n-hexyl);
a is 1, 2, 3, 4, 5 or 6;
b is 0, 1, 2, or 3; and
Z¹ and Z² are each, independently, C₈-C₁₄ alkyl or C₈-C₁₄ alkenyl, wherein the alkenyl group may optionally be substituted with one or two fluorine atoms at the alpha position to a double bond which is between the double bond and the terminus of Z¹ or Z²

In some embodiments, the lipids disclosed in US 2013/0195920 are of formula (II), which has a branched alkyl at the alpha position adjacent to the biodegradable group (between the biodegradable group and the terminus of the tail, i.e., Z¹ o Z²):

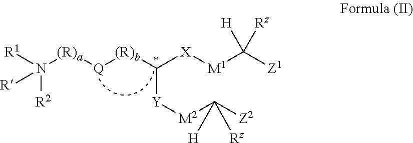

or a salt thereof (e.g., a pharmaceutically acceptable salt thereof), wherein
R′ is absent, hydrogen, or alkyl (e.g., C₁-C₄ alkyl);
with respect to R¹ and R²,
(i) R¹ and R² are each, independently, optionally substituted alkyl, alkenyl, alkynyl, cycloalkylalkyl, heterocycle, or R¹⁰;
(ii) R¹ and R², together with the nitrogen atom to which they are attached, form an optionally substituted heterocylic ring; or
(iii) one of R¹ and R² is optionally substituted alkyl, alkenyl, alkynyl, cycloalkyl, cycloalkylalkyl, or heterocycle, and the other forms a 4-10 member heterocyclic ring or heteroaryl (e.g., a 6-member ring) with (a) the adjacent nitrogen atom and (b) the (R)ₐ group adjacent to the nitrogen atom;
each occurrence of R is, independently, —(CR³R⁴)—;
each occurrence of R³ and R⁴ are, independently H, halogen, OH, alkyl, alkoxy, —NH₂, R¹⁰, alkylamino, or dialkylamino (In some embodiments, each occurrence of R³ and R⁴ are, independently H or C₁-C₄ alkyl);
each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups (preferably at most one R¹⁰ group);
the dashed line to Q is absent or a bond;
when the dashed line to Q is absent then Q is absent or is —O—, —NH—, —S—, —C(O)—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—; or
when the dashed line to Q is a bond then (i) b is 0 and (ii) Q and the tertiary carbon adjacent to it (C*) form a substituted or unsubstituted, mono- or bi-cyclic heterocyclic group having from 5 to 10 ring atoms (e.g., the heteroatoms in the heterocyclic group are selected from O and S, preferably 0);
each occurrence of R⁵ is, independently, H or alkyl;
X and Y are each, independently, alkylene (e.g., C₆-C₈ alkylene) or alkenylene, wherein the alkylene or alkenylene group is optionally substituted with one or two fluorine atoms at the alpha position to the M¹ or M² group
M¹ and M² are each, independently, a biodegradable group (e.g., —OC(O)—, —C(O)O—, —SC(O)—, —C(O)S—, —OC(S)—, —C(S)O, —S—S—, —C(R⁵)═N N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

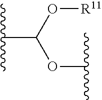

(wherein R¹¹ is a C₂-C₈ alkyl or alkenyl));
each occurrence of R^{z} is, independently, C₁-C₈ alkyl (e.g., methyl, ethyl, isopropyl);
a is 1, 2, 3, 4, 5 or 6;
b is 0, 1, 2, or 3; and
Z¹ and Z² are each, independently, C₈-C₁₄ alkyl or C₈-C₁₄ alkenyl, wherein (i) the alkenyl group may optionally be substituted with one or two fluorine atoms at the alpha position to a double bond which is between the double bond and the terminus of Z¹ or Z²
and (ii) the terminus of at least one of Z¹ and Z² is separated from the group M¹ or M² by at least 8 carbon atoms.

In some embodiments, the lipids disclosed in US 2013/0195920 are of formula (III), which has a branching point at a position that is 2-6 carbon atoms (i.e., at the beta (β), gamma (γ), delta (δ), epsilon (ε) or zeta position (ζ) adjacent to the biodegradable group (between the biodegradable group and the terminus of the tail, i.e., Z¹ or Z²):

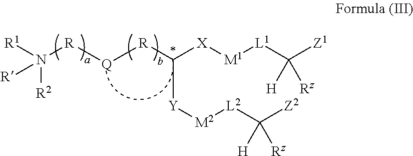

or a salt thereof (e.g., a pharmaceutically acceptable salt thereof), wherein
R′, R¹, R², R, R³, R⁴, R¹⁰, Q, R⁵, M¹, M², R^{z}, a, and b are defined as in formula (I);
L¹ and L² are each, independently, C₁-C₅ alkylene or C₂-C₅ alkenylene;
X and Y are each, independently, alkylene (e.g., C₄ to C₂₀ alkylene or C₆-C₈ alkylene) or alkenylene (e.g., C₄ to C₂₀ alkenylene); and
Z¹ and Z² are each, independently, C₈-C₁₄ alkyl or C₈-C₁₄ alkenyl, wherein the alkenyl group may optionally be substituted with one or two fluorine atoms at the alpha position to a double bond which is between the double bond and the terminus of Z¹ or Z²
and with the proviso that the terminus of at least one of Z¹ and Z² is separated from the group M¹ or M² by at least 8 carbon atoms.

In some embodiments, the lipids disclosed in US 2013/0195920 include, but are not limited to,

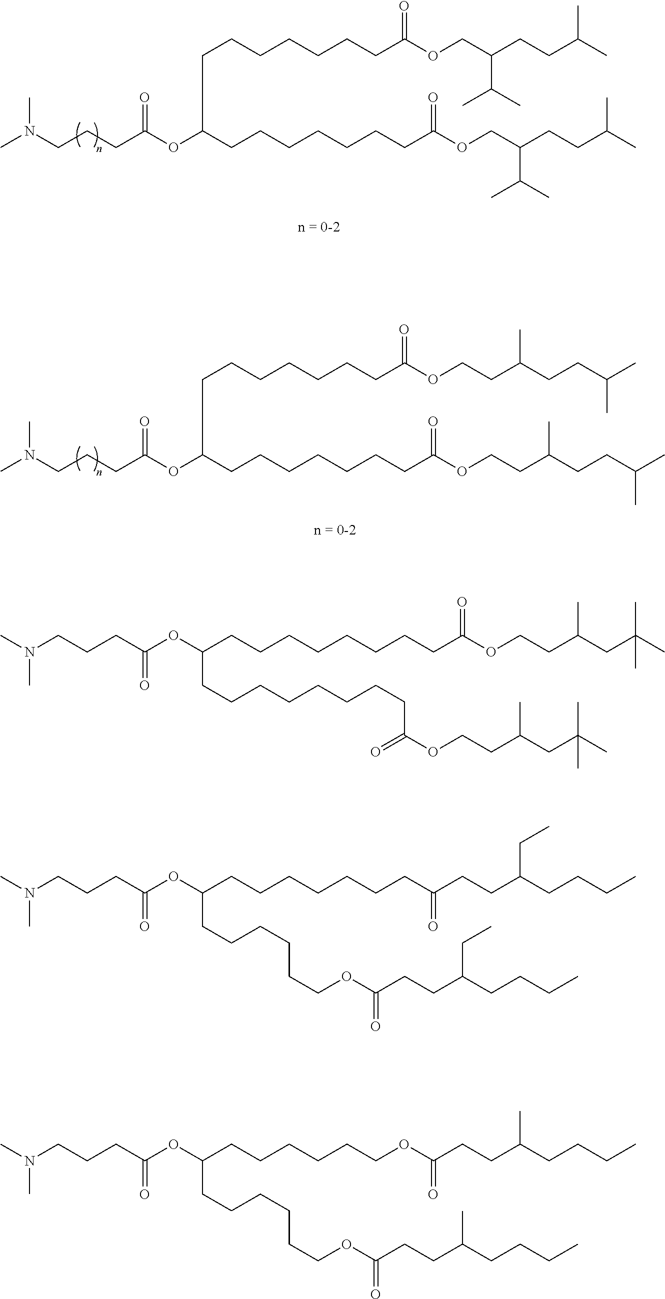

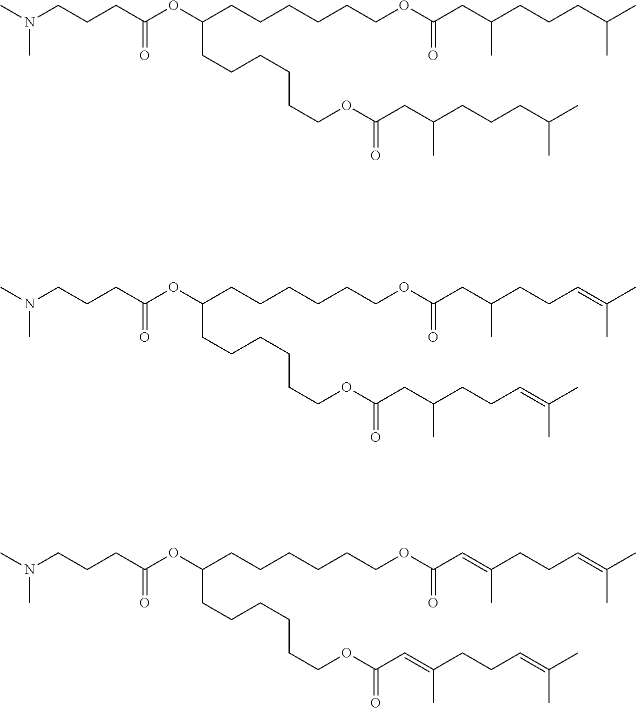

In some embodiments, the cationic lipid disclosed in US 2013/0195920 is a compound of formula (IV), which has a branching point at a position that is 2-6 carbon atoms (i.e., at beta (β), gamma (γ), delta (δ), epsilon (ε) or zeta position (ζ) adjacent to the biodegradable group (between the biodegradable group and the terminus of the tail, i.e., Z¹ or Z²):

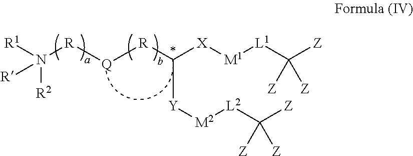

or a salt thereof (e.g., a pharmaceutically acceptable salt thereof), wherein
R′, R¹, R², R, R³, R⁴, R¹⁰, Q, R⁵, M², R^{z}, a, and b are defined as in formula (I);
L¹ and L² and are each, independently, C₁-C₅ alkylene or C₂-C₅ alkenylene;
X and Y are each, independently, alkylene or alkenylene (e.g., C₁₂-C₂₀ alkylene or C₁₂-C₂₀ alkenylene); and
each occurrence of Z is independently C₁-C₄ alkyl (preferably, methyl).

For example, in some embodiments, -L¹-C(Z)₃ is —CH₂C(CH₃)₃. In some embodiments, -L¹-C(Z)₃ is —CH₂CH₂C(CH₃)₃.

In some embodiments, the lipids disclosed in US 2013/0195920 are of formula (V), which has an alkoxy or thioalkoxy (i.e., —S-alkyl) group substitution on at least one tail:

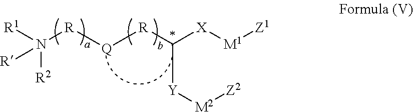

or a salt thereof (e.g., a pharmaceutically acceptable salt thereof), wherein
R′, R¹, R², R, R³, R⁴, R¹⁰, Q, R⁵, M¹, M², a, and b are defined as in formula (I);
X and Y are each, independently, alkylene (e.g., C₆-C₈ alkylene) or alkenylene, wherein the alkylene or alkenylene group is optionally substituted with one or two fluorine atoms at the alpha position to the M¹ or M² group
Z¹ and Z² are each, independently, C₅-C₁₄ alkyl or C₅-C₁₄ alkenyl, wherein (i) the C₈-C₁₄ alkyl or C₅-C₁₄ alkenyl of at least one of Z¹ and Z² is substituted by one or more alkoxy (e.g., a C₁-C₄ alkoxy such as —OCH₃) or thioalkoxy (e.g., a C₁-C₄ thioalkoxy such as —SCH₃) groups, and (ii) the alkenyl group may optionally be substituted with one or two fluorine atoms at the alpha position to a double bond which is between the double bond and the terminus of Z¹ or Z²

In some embodiments, the lipids disclosed in US 2013/0195920 are of formula (VIA), which has one or more fluoro substituents on at least one tail at a position that is either alpha to a double bond or alpha to a biodegradable group:

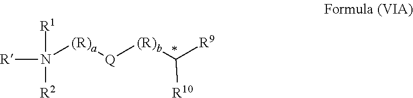

or a salt thereof (e.g., a pharmaceutically acceptable salt thereof), wherein
R¹, R², R, a, and b are as defined with respect to formula (I);
Q is absent or is —O—, —NH—, —S—, —C(O)—, —C(O)O—, —OC(O)—, —C(O)N(R⁴)—, —N(R⁵)C(O)—, —S—S—, OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;
R′ is absent, hydrogen, or alkyl (e.g., C₁-C₄ alkyl); and
each of R⁹ and R¹⁰ are independently C₁₂-C₂₄ alkyl (e.g., C₁₂-C₂₀ alkyl), C₁₂-C₂₄ alkenyl (e.g., C₁₂-C₂₀) alkenyl), or C₁₂-C₂₄ alkoxy (e.g., C₁₂-C₂₀) alkoxy) (a) having one or more biodegradable groups and (b) optionally substituted with one or more fluorine atoms at a position which is (i) alpha to a biodegradable group and between the biodegradable group and the tertiary carbon atom marked with an asterisk (*), or (ii) alpha to a carbon-carbon double bond and between the double bond and the terminus of the R⁹ or R¹⁰ group; each biodegradable group independently interrupts the C₁₂-C₂₄ alkyl, alkenyl, or alkoxy group or is substituted at the terminus of the C₁₂-C₂₄ alkyl, alkenyl, or alkoxy group, wherein
(i) at least one of R⁹ and R¹⁰ contains a fluoro group;
(ii) the compound does not contain the following moiety:

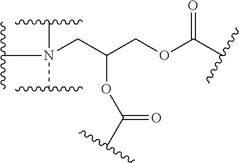

wherein [symbol] is an optional bond; and
(iii) the terminus of R⁹ and R¹⁰ is separated from the tertiary carbon atom marked with an asterisk (*) by a chain of 8 or more atoms (e.g., 12 or 14 or more atoms).

In some embodiments, the terminus of R⁹ and R¹⁰ is separated from the tertiary carbon atom marked with an asterisk (*) by a chain of 18-22 carbon atoms (e.g., 18-20 carbon atoms).

In some embodiments, the lipids disclosed in US 2013/0195920 are of formula (VIB), which has one or more fluoro substituents on at least one tail at a position that is either alpha to a double bond or alpha to a biodegradable group:

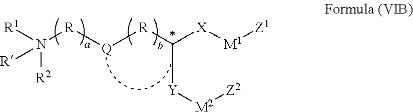

or a salt thereof (e.g., a pharmaceutically acceptable salt thereof), wherein
R′, R¹, R², R, R³, R⁴, R¹⁰, Q, R⁵, M¹, M², a, and b are defined as in formula (I);
X and Y are each, independently, alkylene (e.g., C₆-C₈ alkylene) or alkenylene, wherein the alkylene or alkenylene group is optionally substituted with one or two fluorine atoms at the alpha position to the M¹ or M² group; and
Z¹ and Z² are each, independently, C₈-C₁₄ alkyl or C₈-C₁₄ alkenyl, wherein said C₈-C₁₄ alkenyl is optionally substituted by one or more fluorine atoms at a position that is alpha to a double bond,
wherein at least one of X, Y, Z¹, and Z² contains a fluorine atom.

In some embodiments, the lipids disclosed in US 2013/0195920 are of formula (VII), which has an acetal group as a biodegradable group in at least one tail:

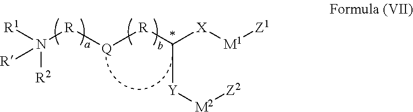

or a salt thereof (e.g., a pharmaceutically acceptable salt thereof), wherein
R′, R¹, R², R, R³, R⁴, R¹⁰, Q, R⁵, a, and b are defined as in formula (I);
X and Y are each, independently, alkylene (e.g., C₆-C₈ alkylene) or alkenylene, wherein the alkylene or alkenylene group is optionally substituted with one or two fluorine atoms at the alpha position to the M¹ or M² group
M¹ and M² are each, independently, a biodegradable group (e.g., —OC(O)—, —C(O)O—, —SC(O)—, —C(O)S—, —OC(S)—, —C(S)O, —S—S—, —C(R⁵)═N N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

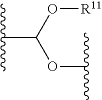

(wherein R″ is a C₄-C₁₀ alkyl or C₄-C₁₀ alkenyl));
with the proviso that at least one of M¹ and M² is

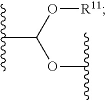

and
Z¹ and Z² are each, independently, C₄-C₁₄ alkyl or C₄-C₁₄ alkenyl, wherein the alkenyl group may optionally be substituted with one or two fluorine atoms at the alpha position to a double bond which is between the double bond and the terminus of Z¹ or Z²

Representative asymmetrical cationic lipids disclosed in US 2013/0195920 include:

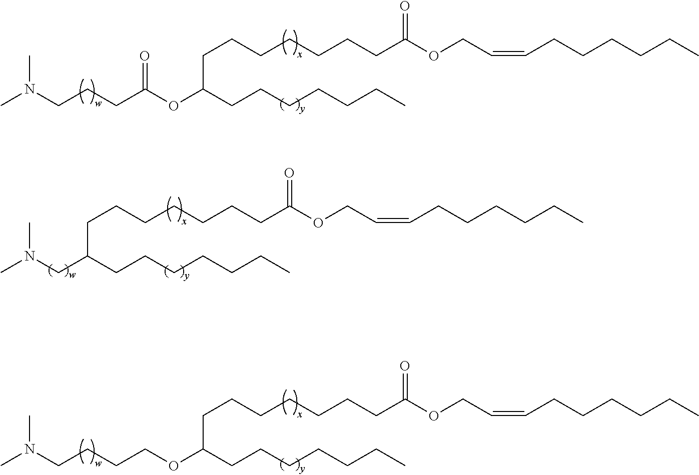

wherein w is 0, 1, 2, or 3; and x and y are each independently 1, 2, 3, 4, 5, 6, or 7.

For instance, the cationic lipid can be:

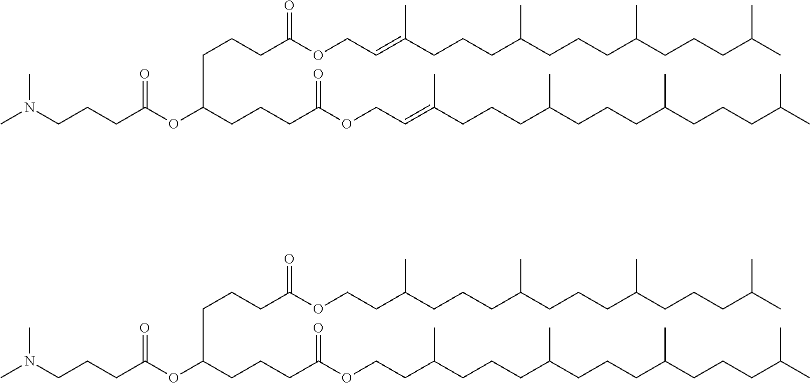

Other cationic lipids disclosed in US 2013/0195920 include, but are not limited to:

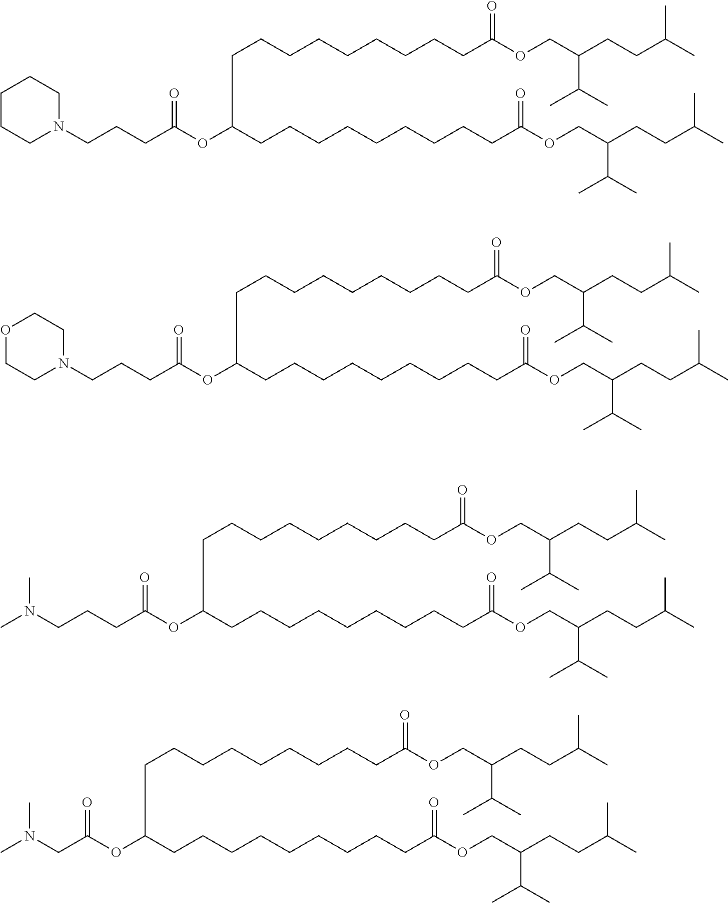

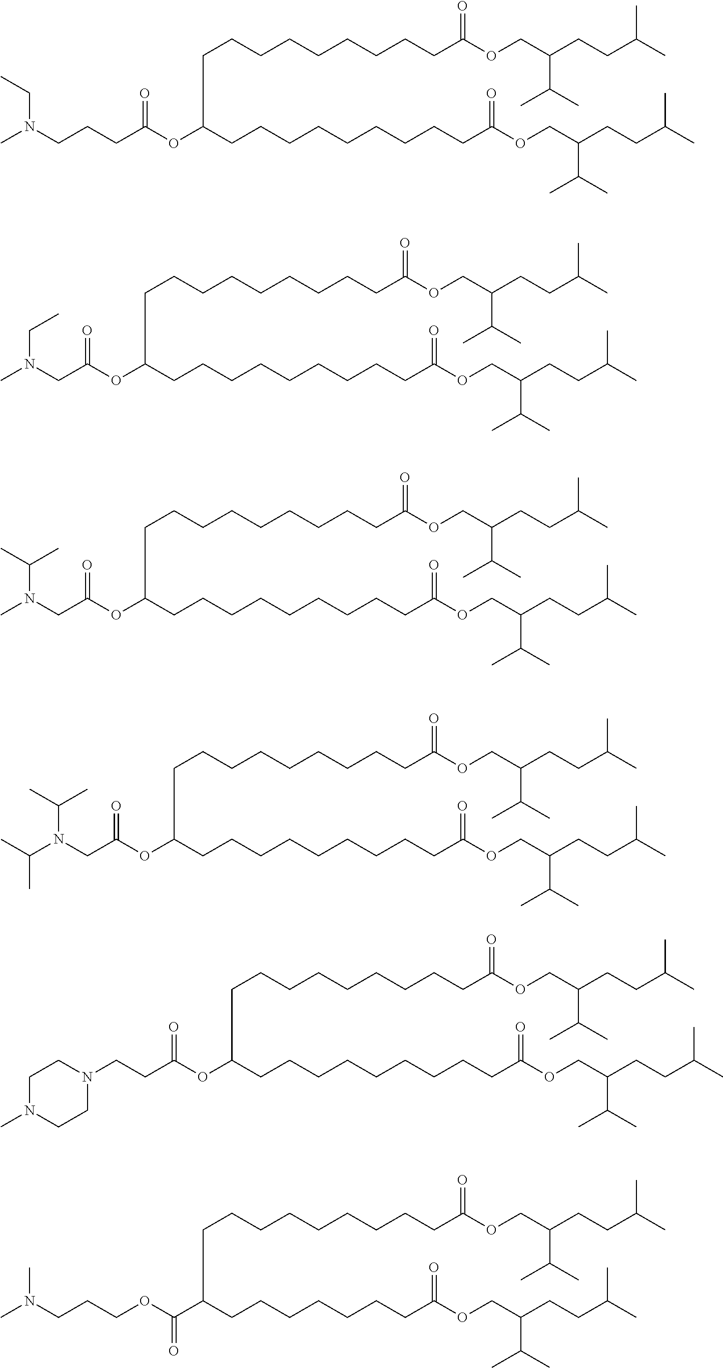

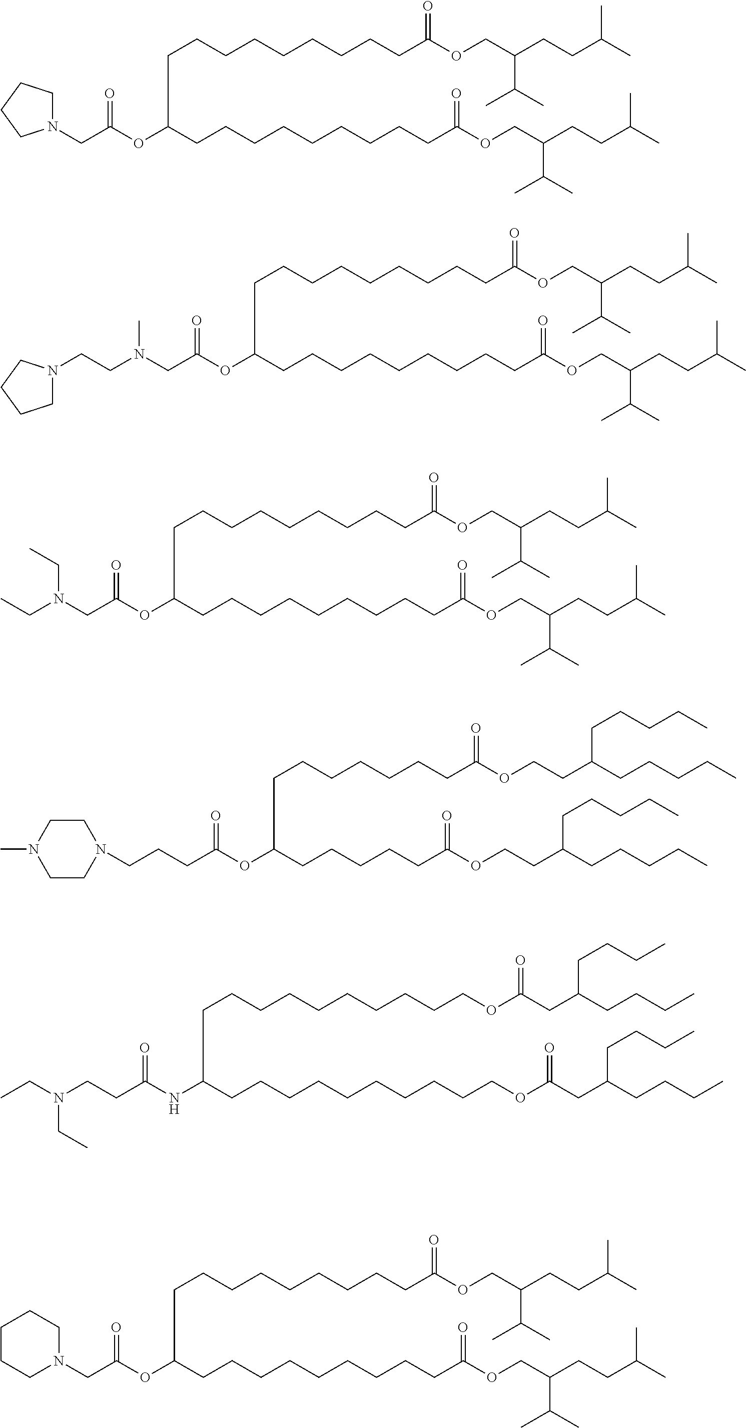

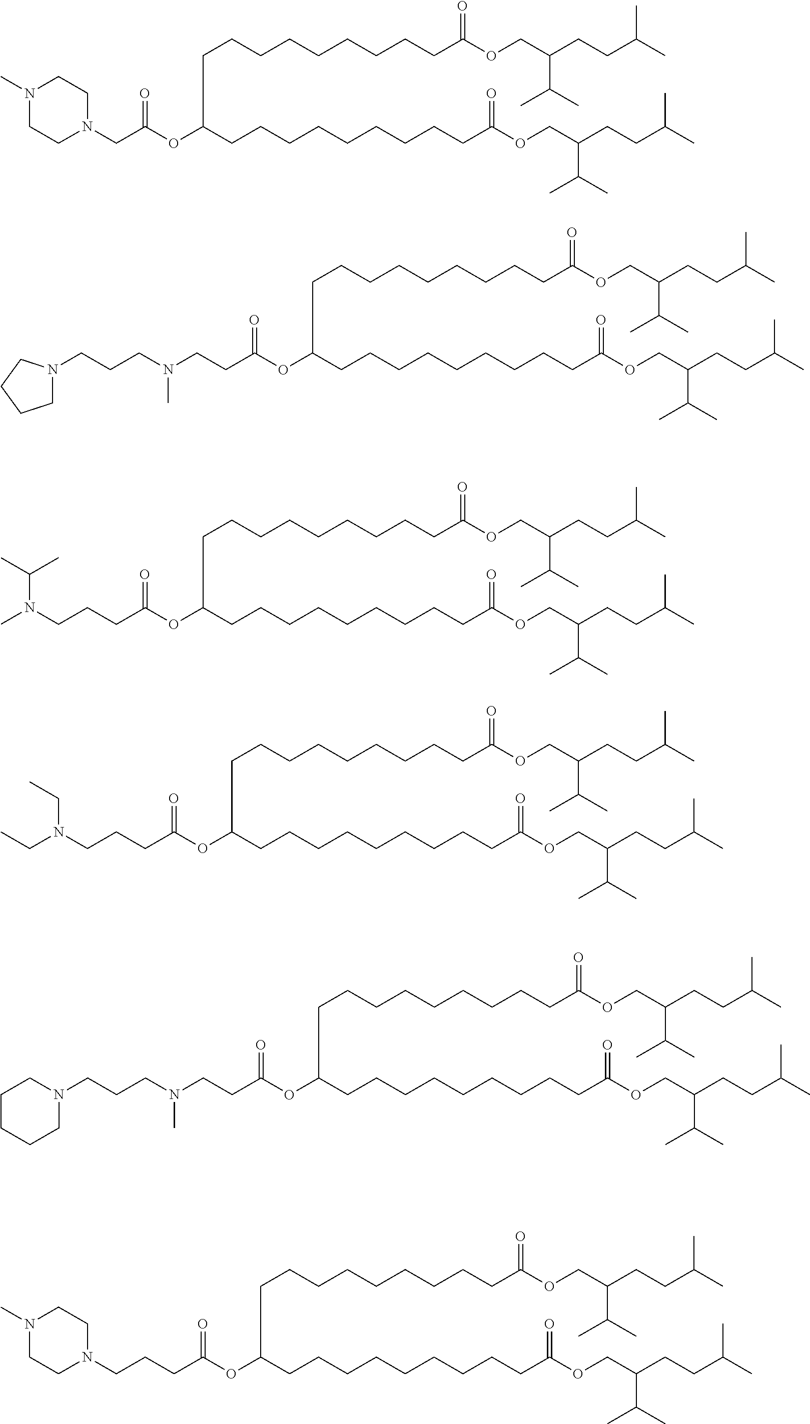

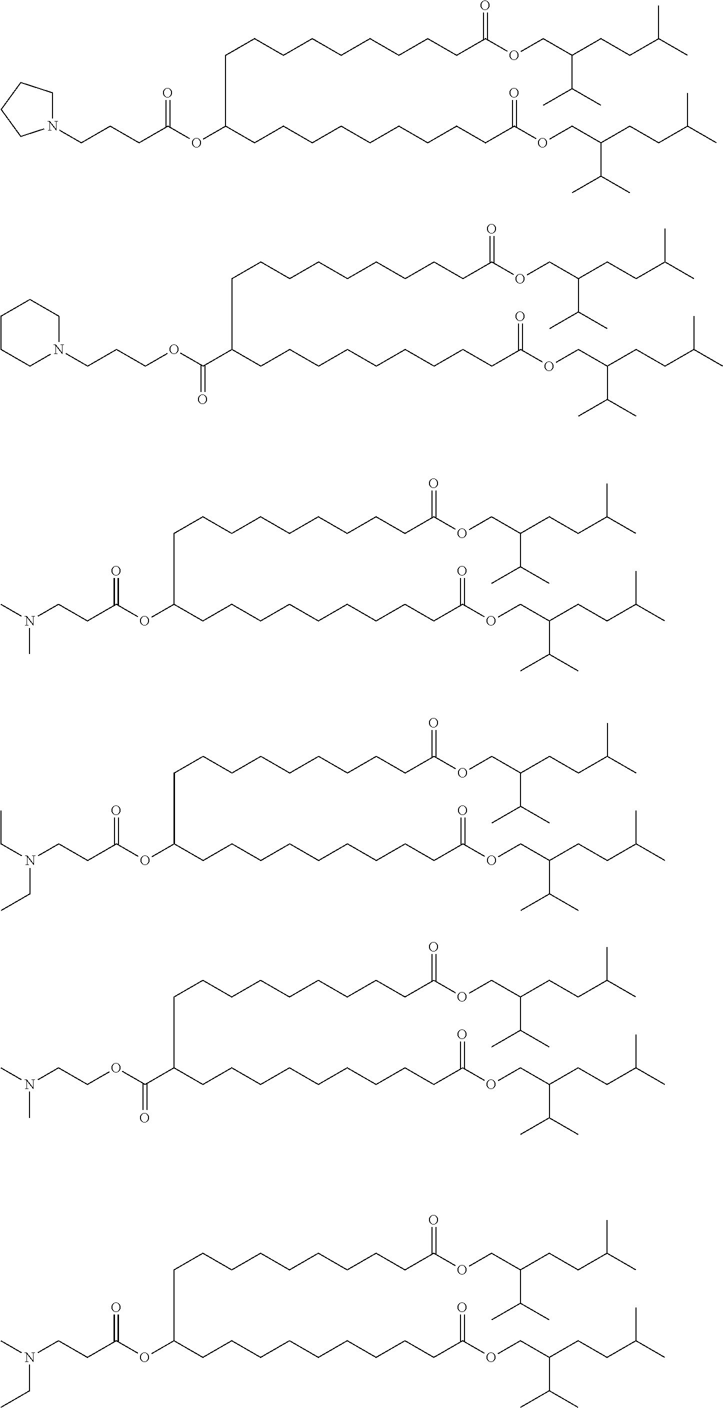

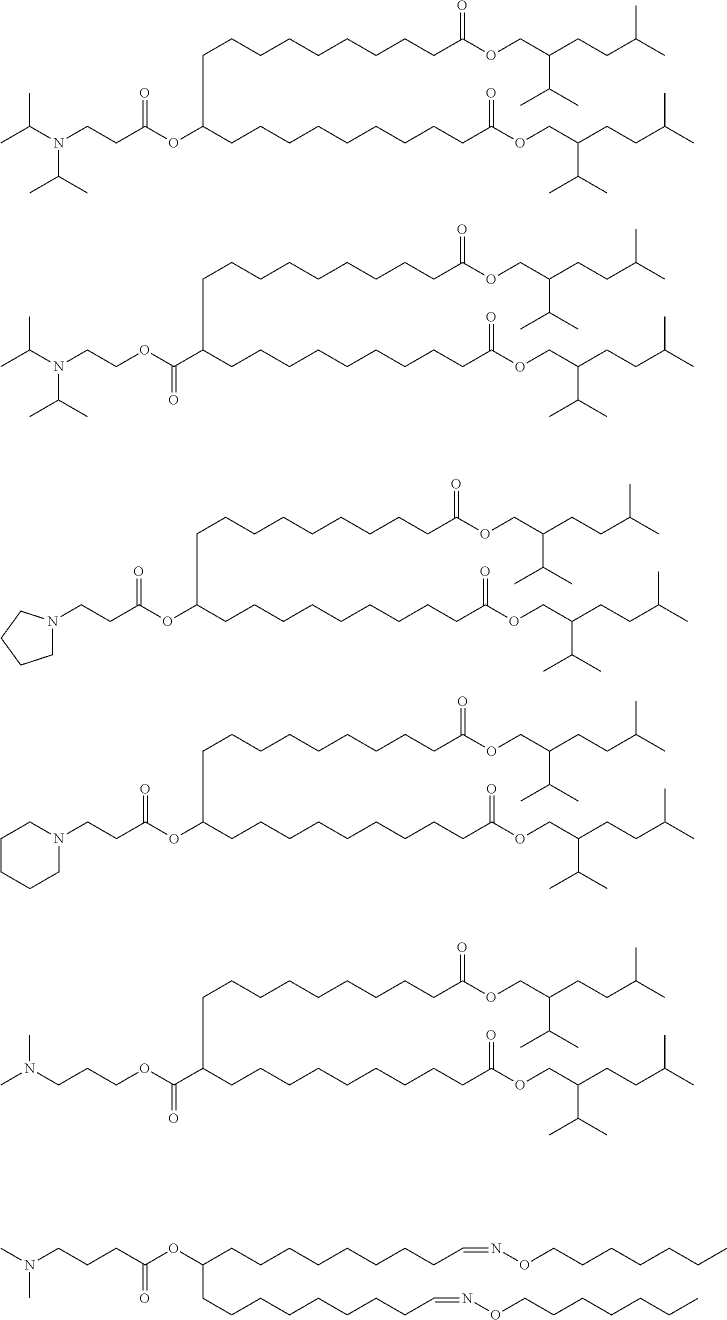

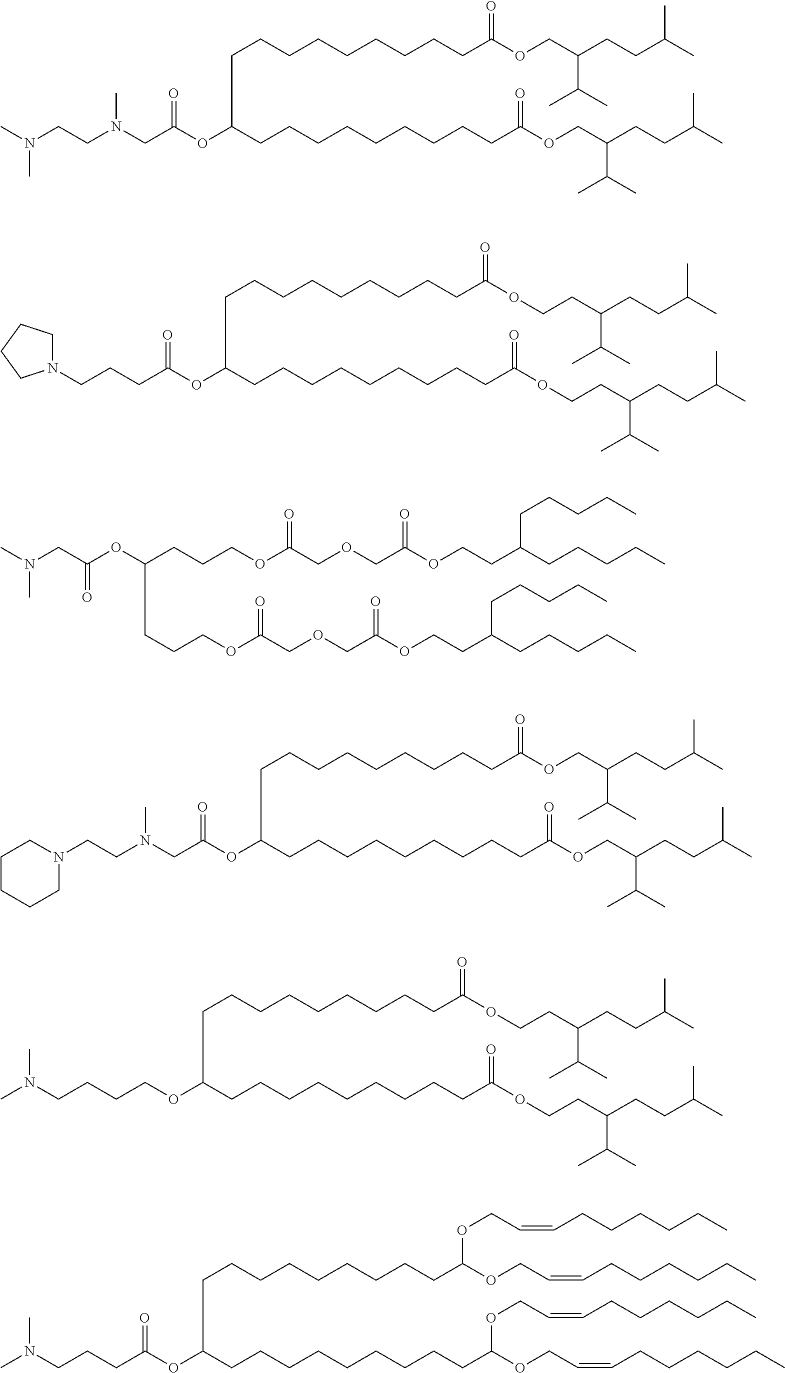

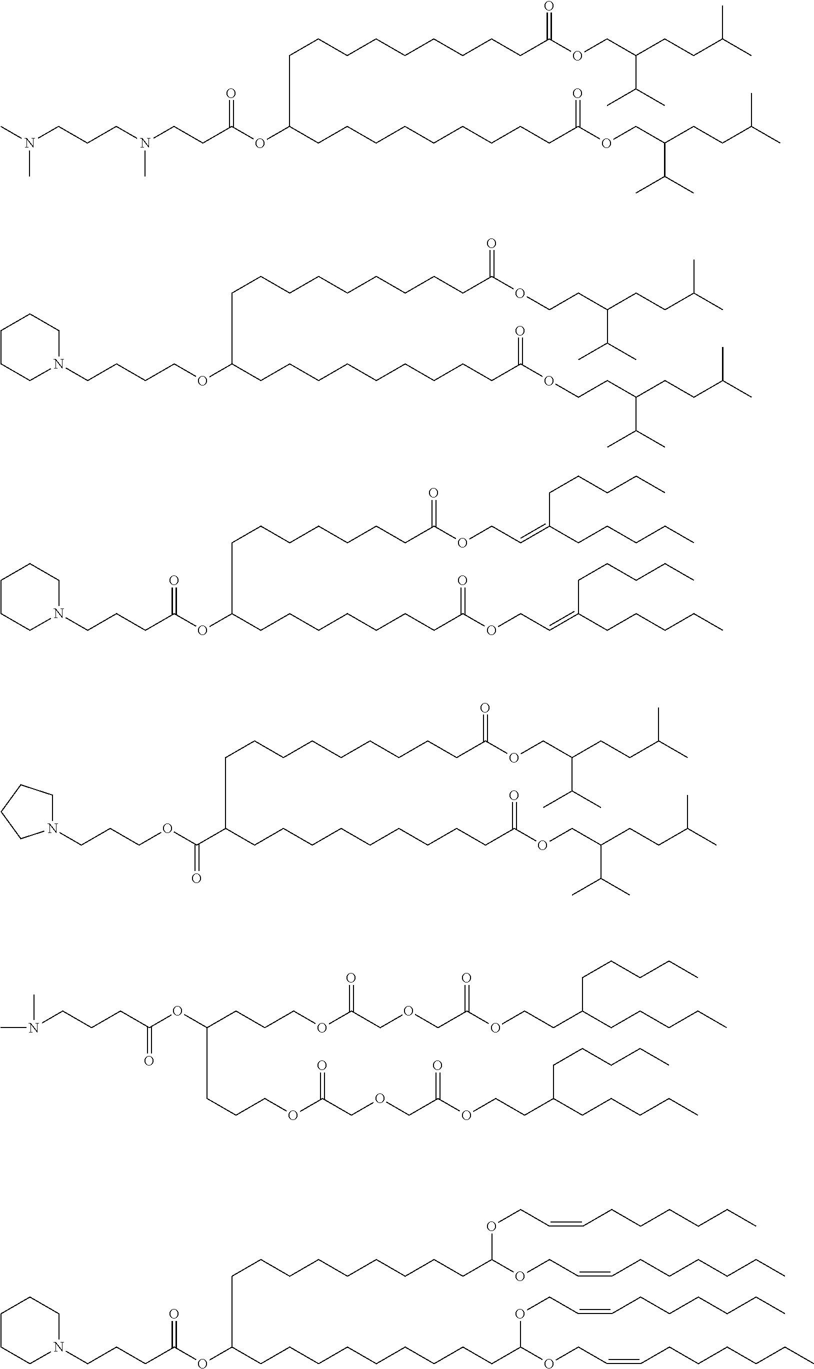

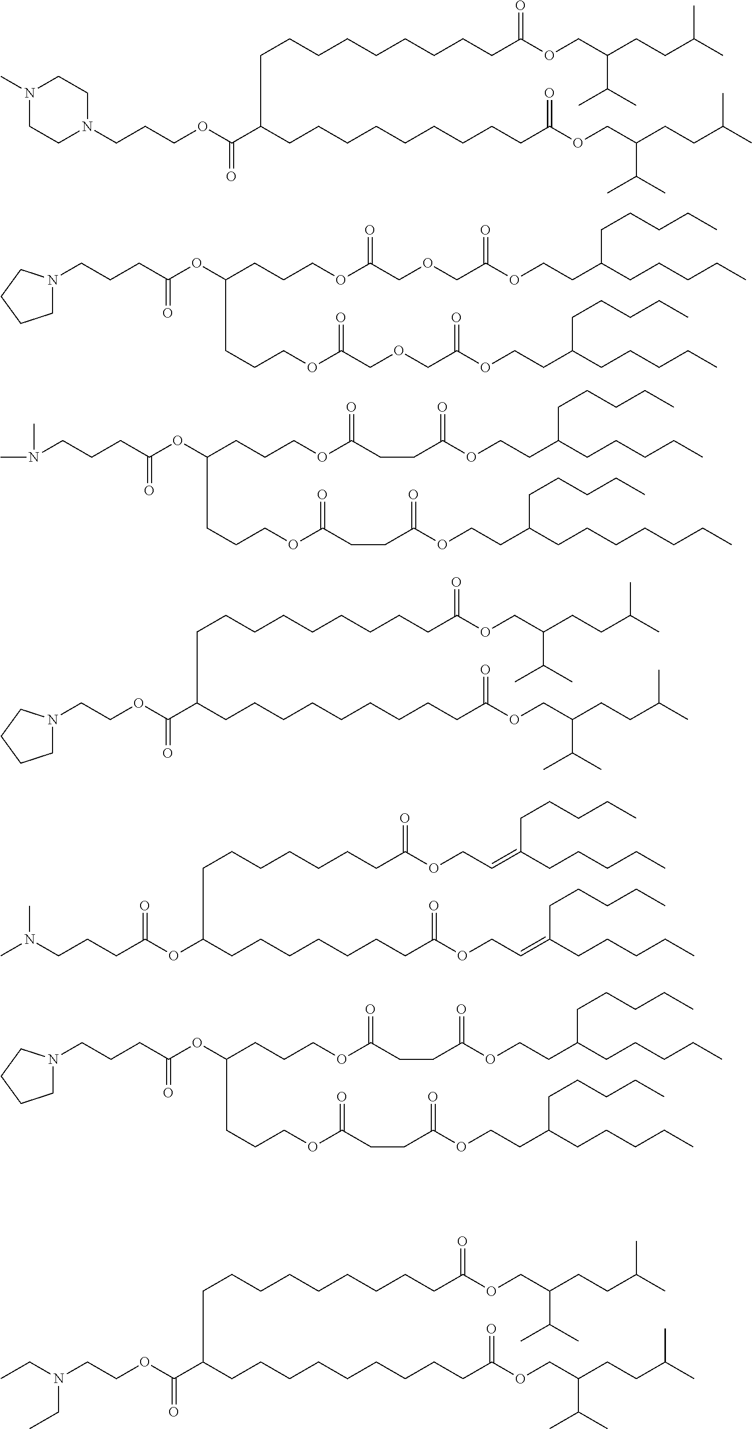

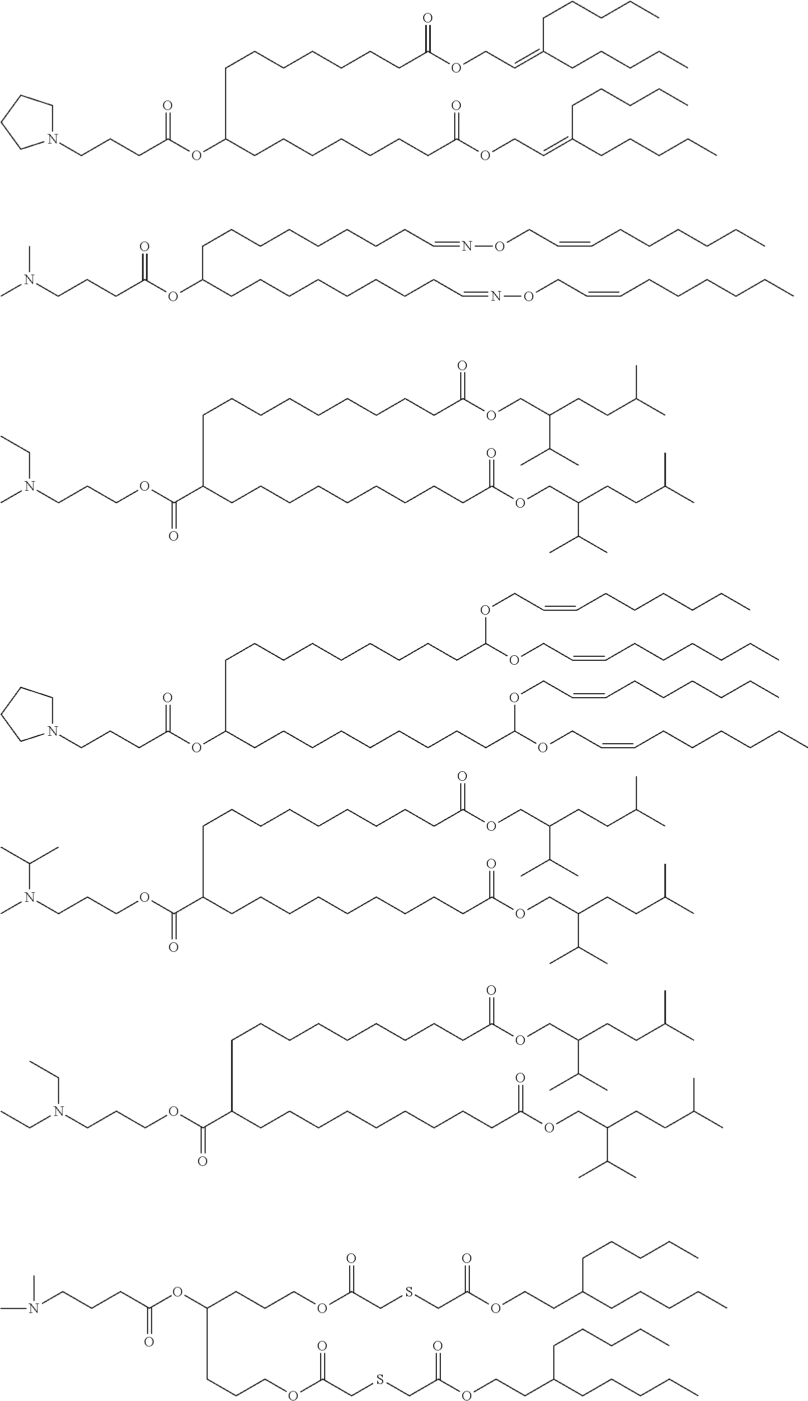

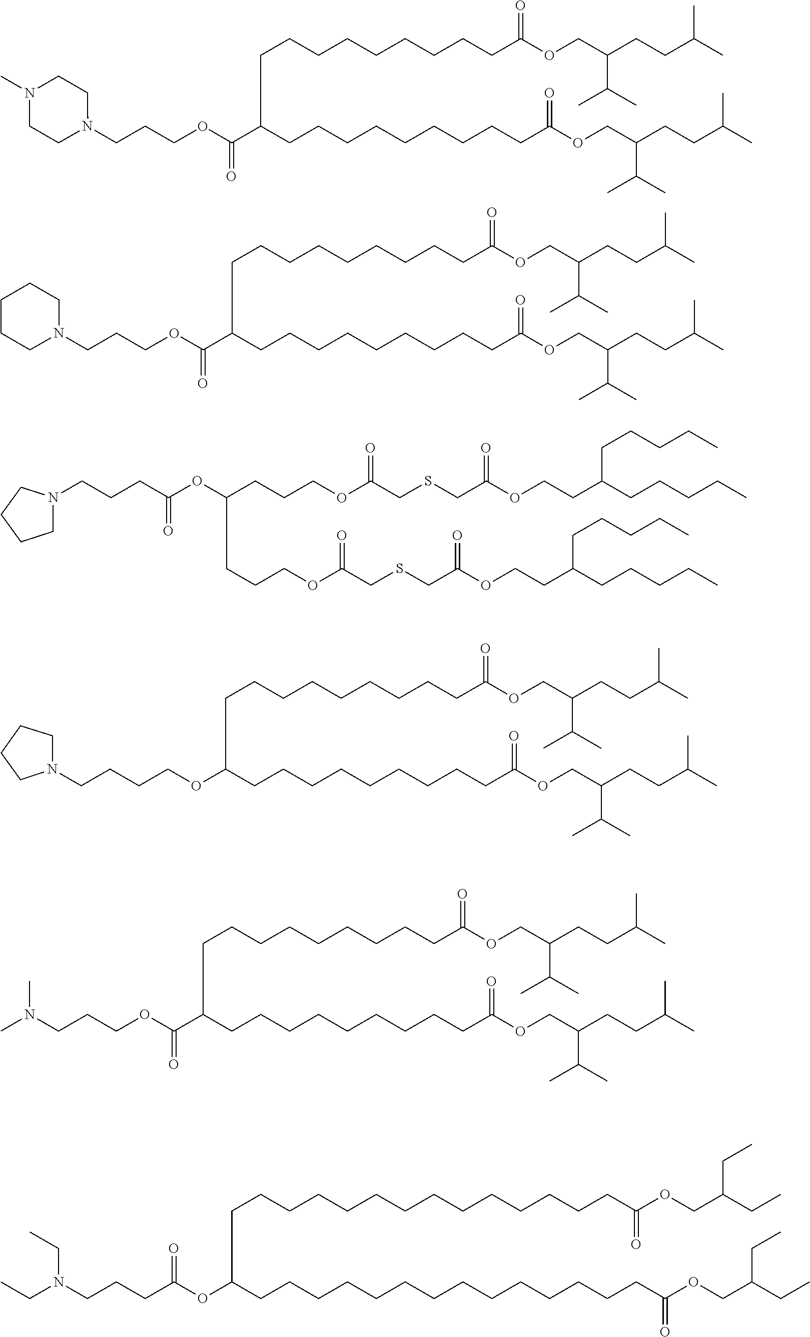

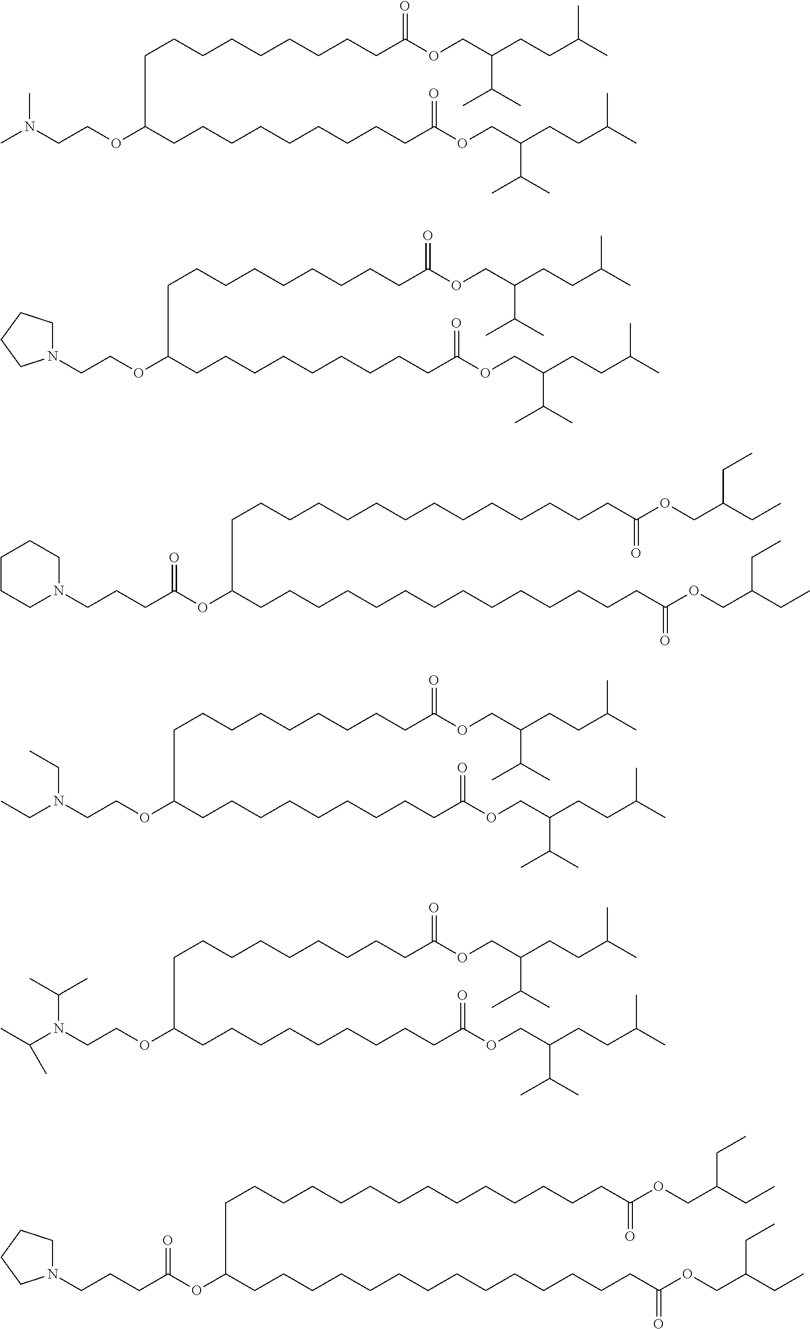

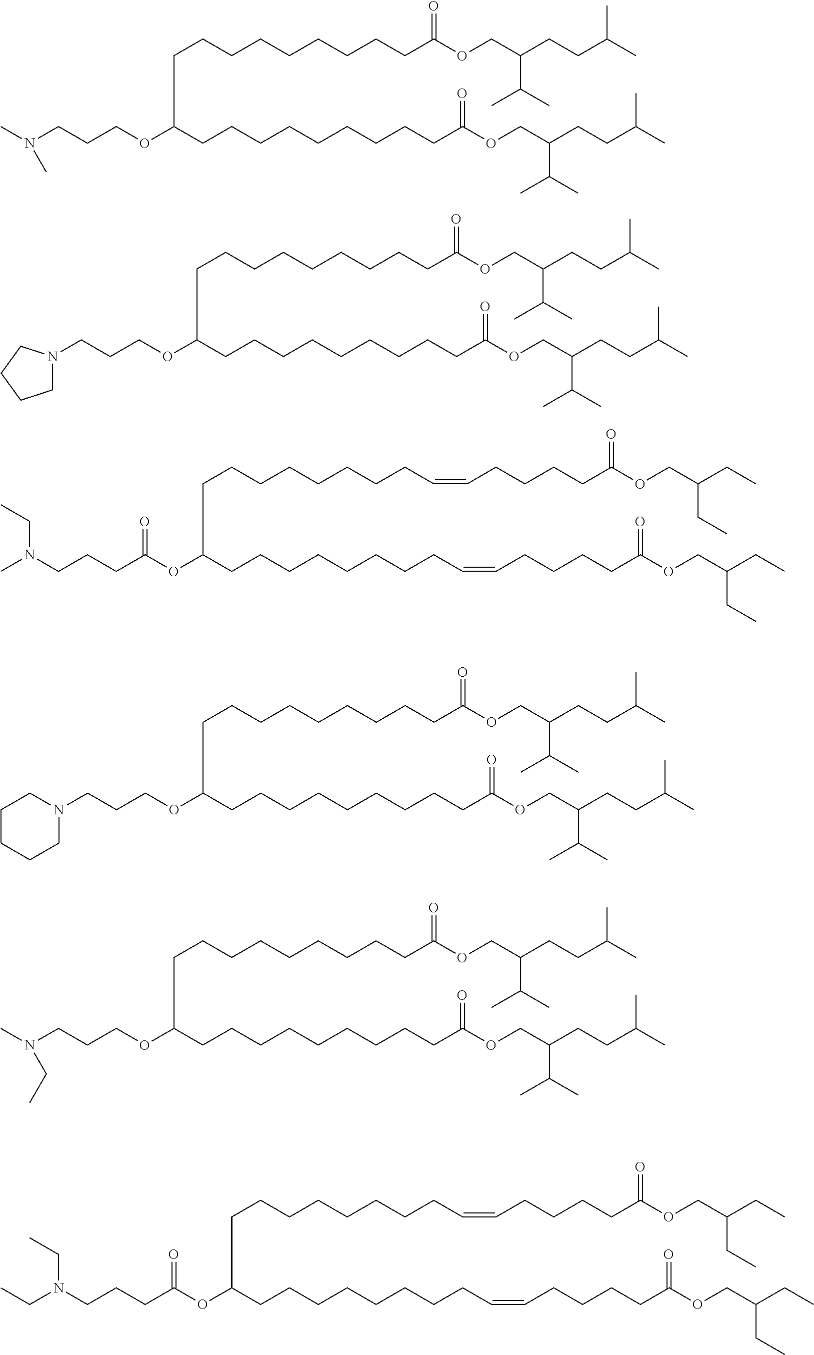

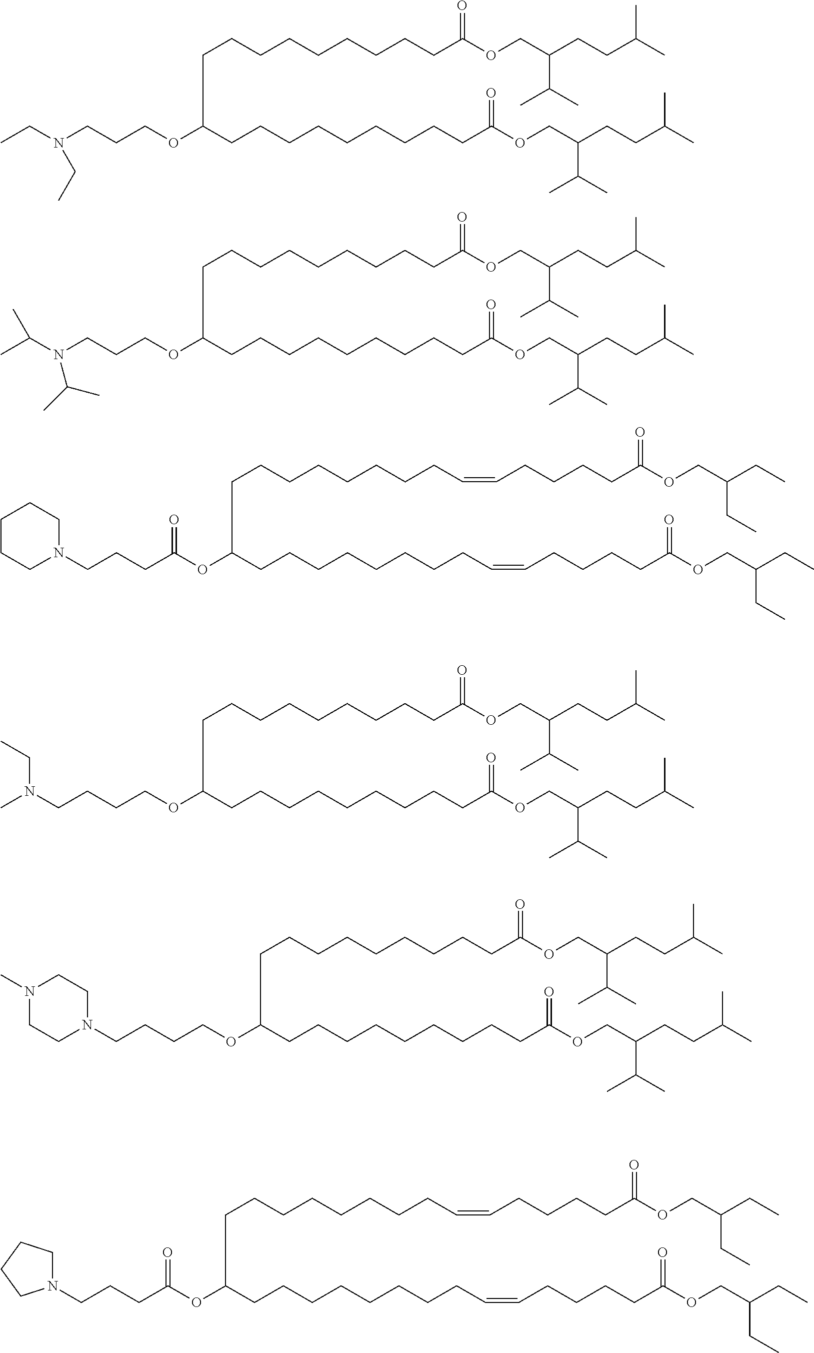

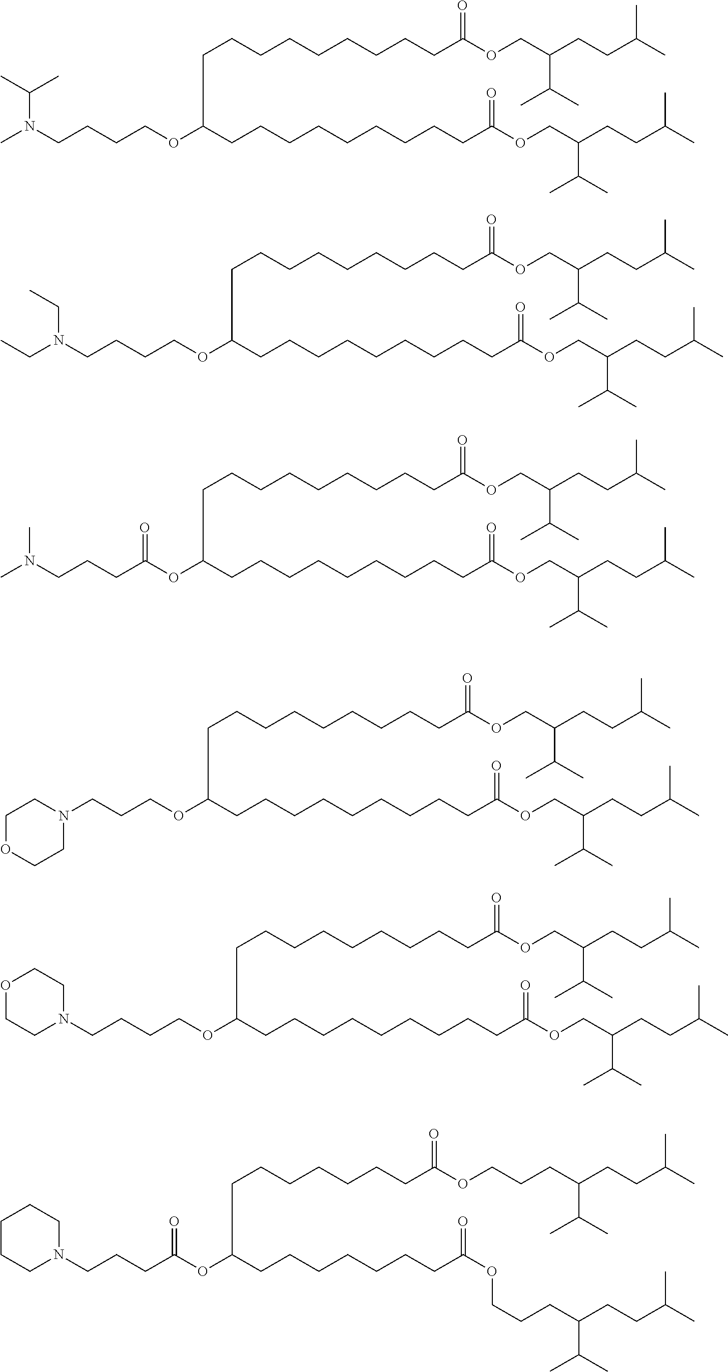

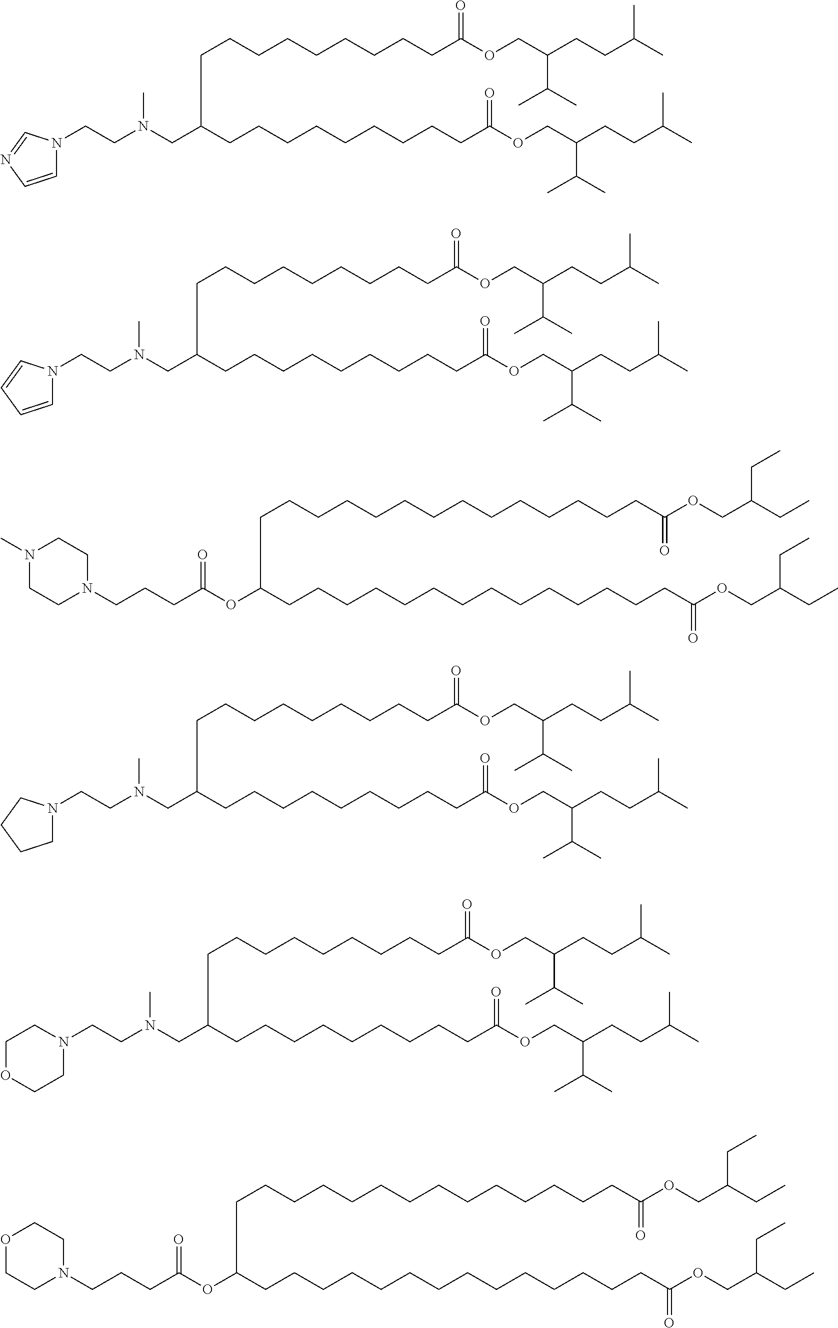

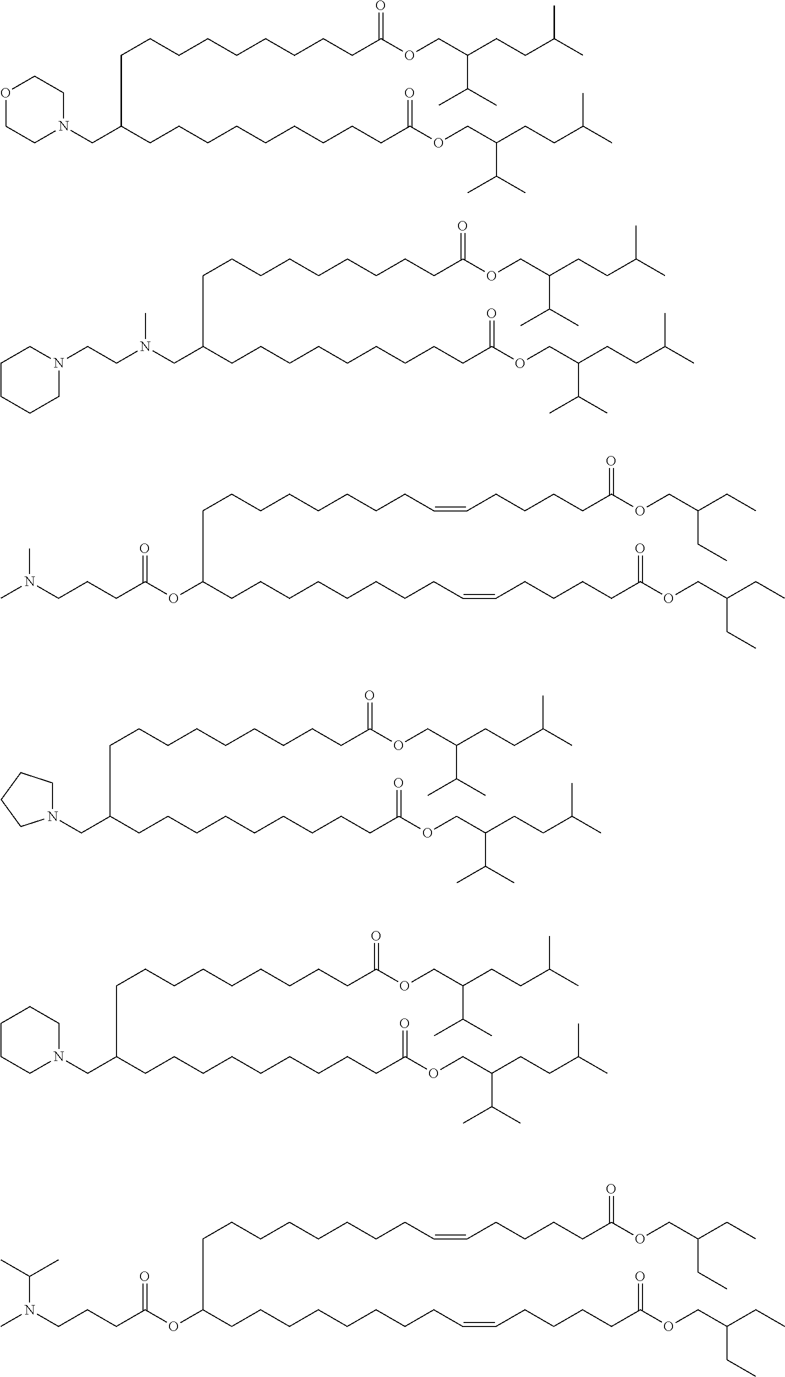

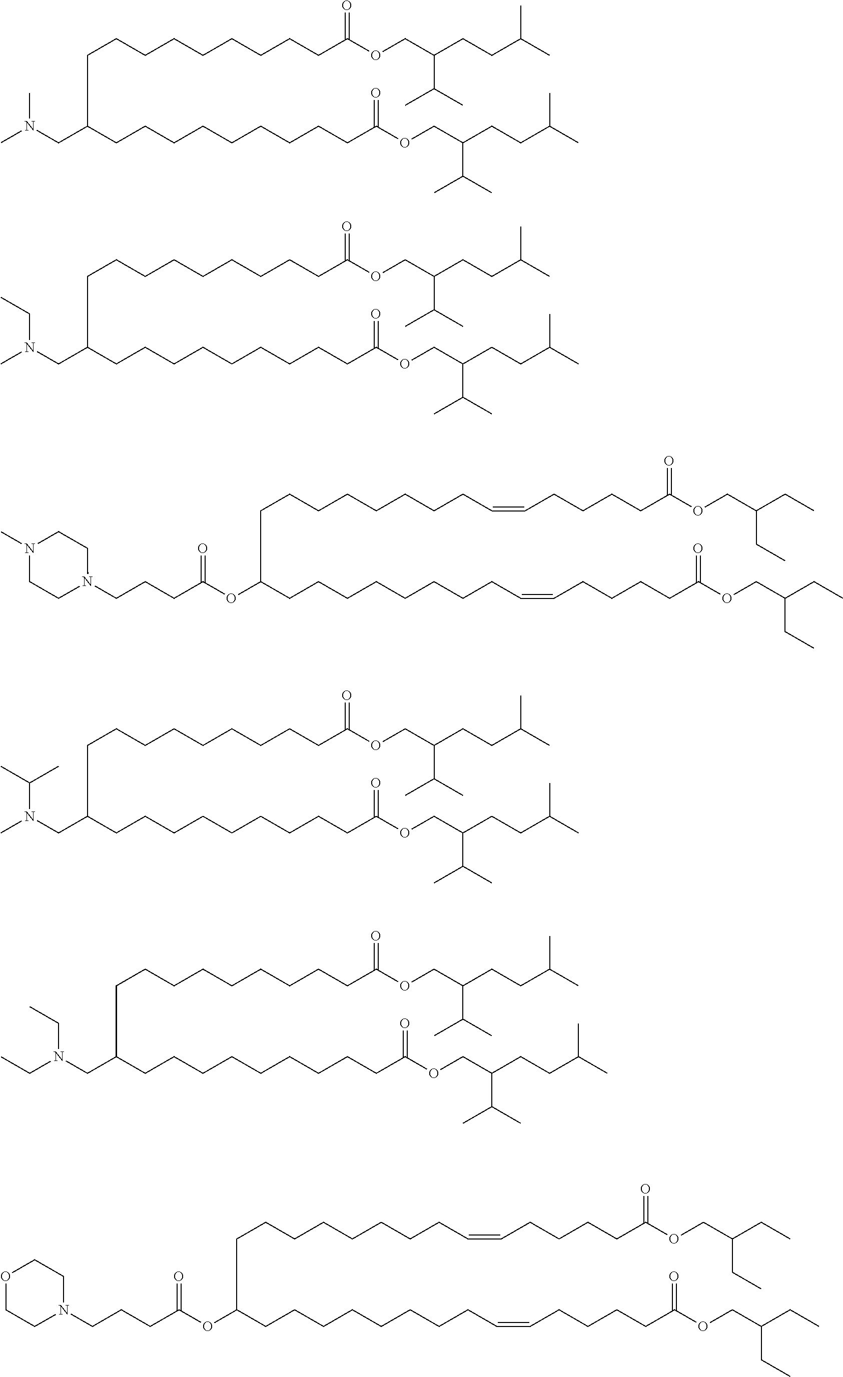

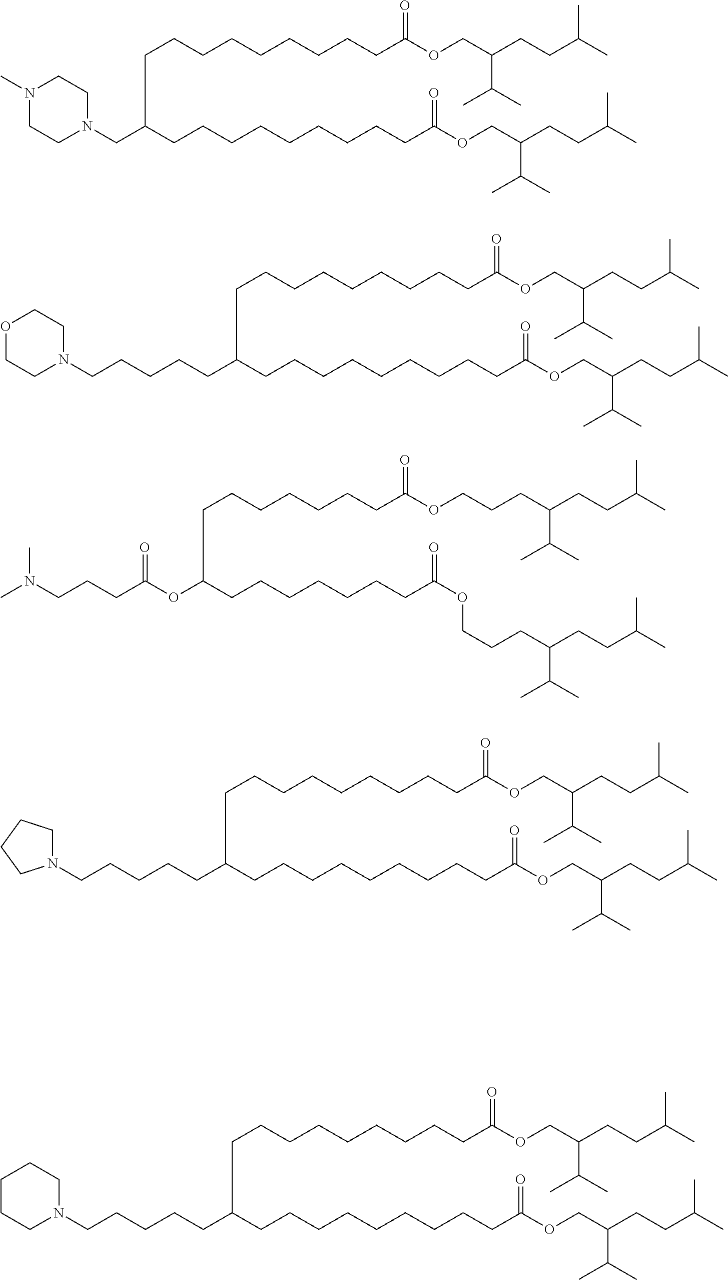

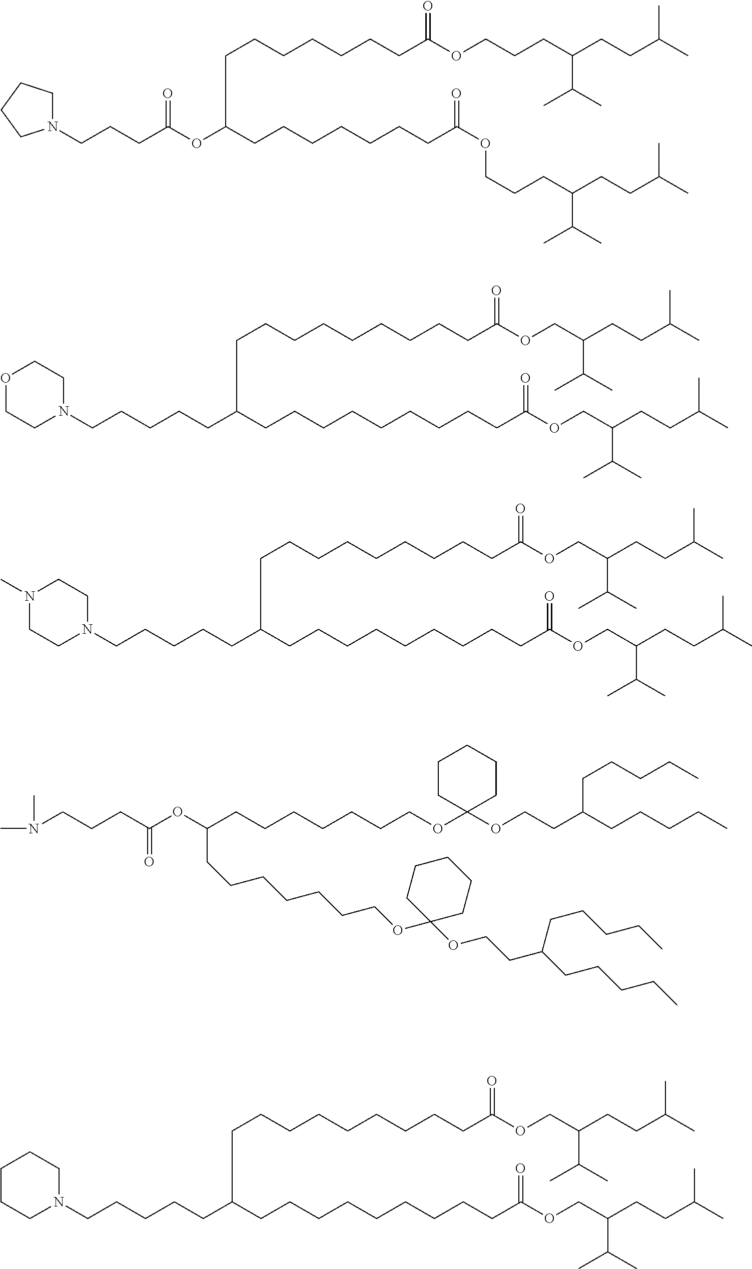

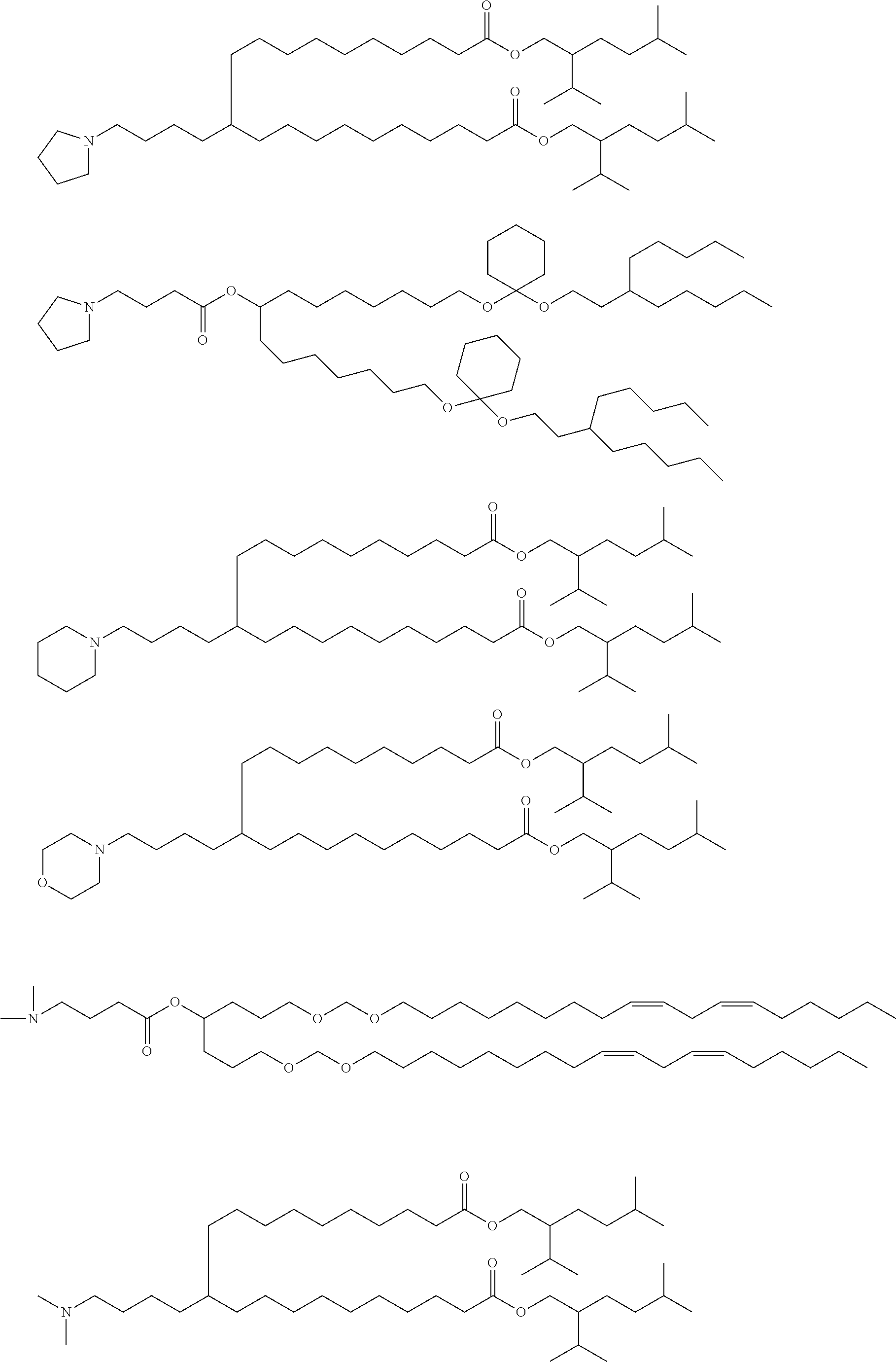

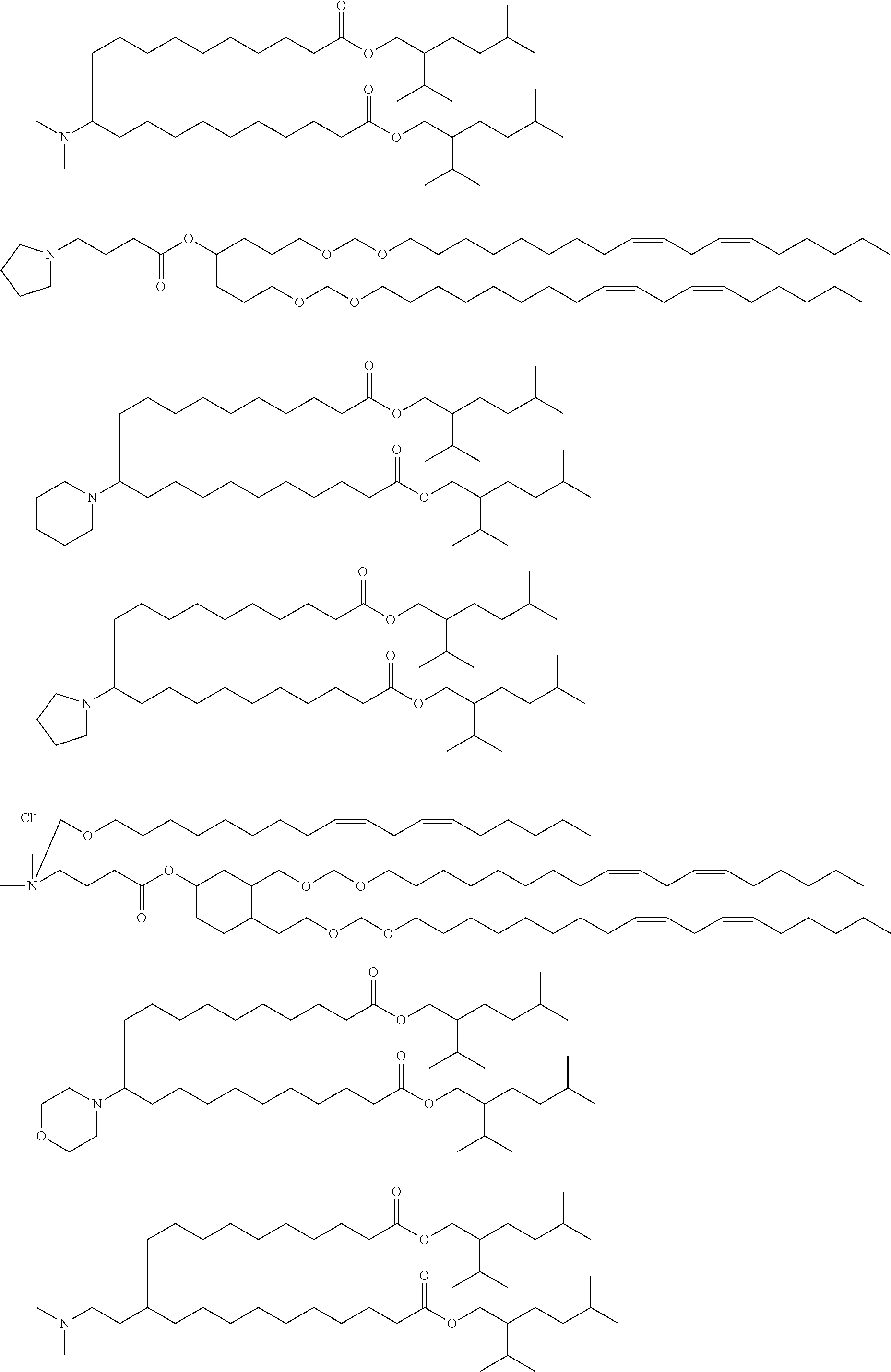

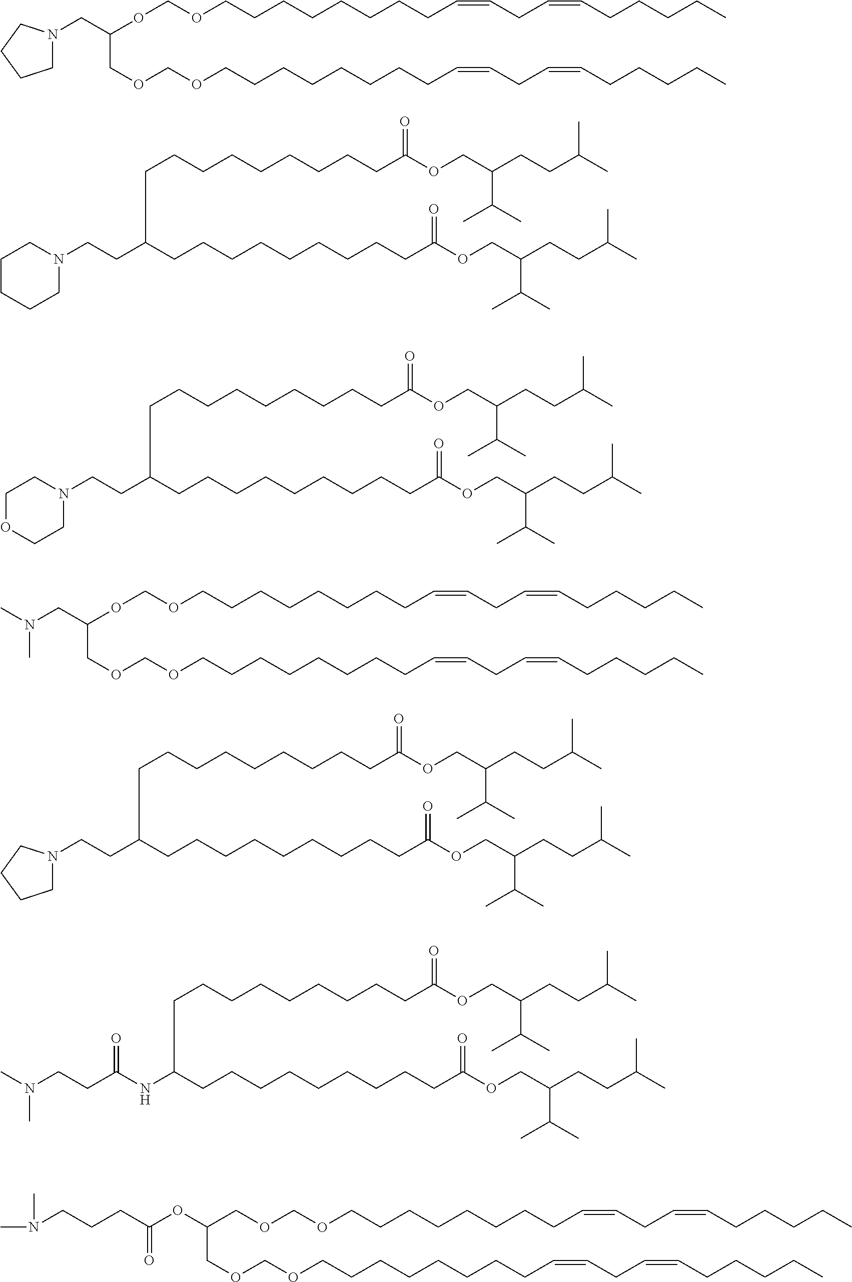

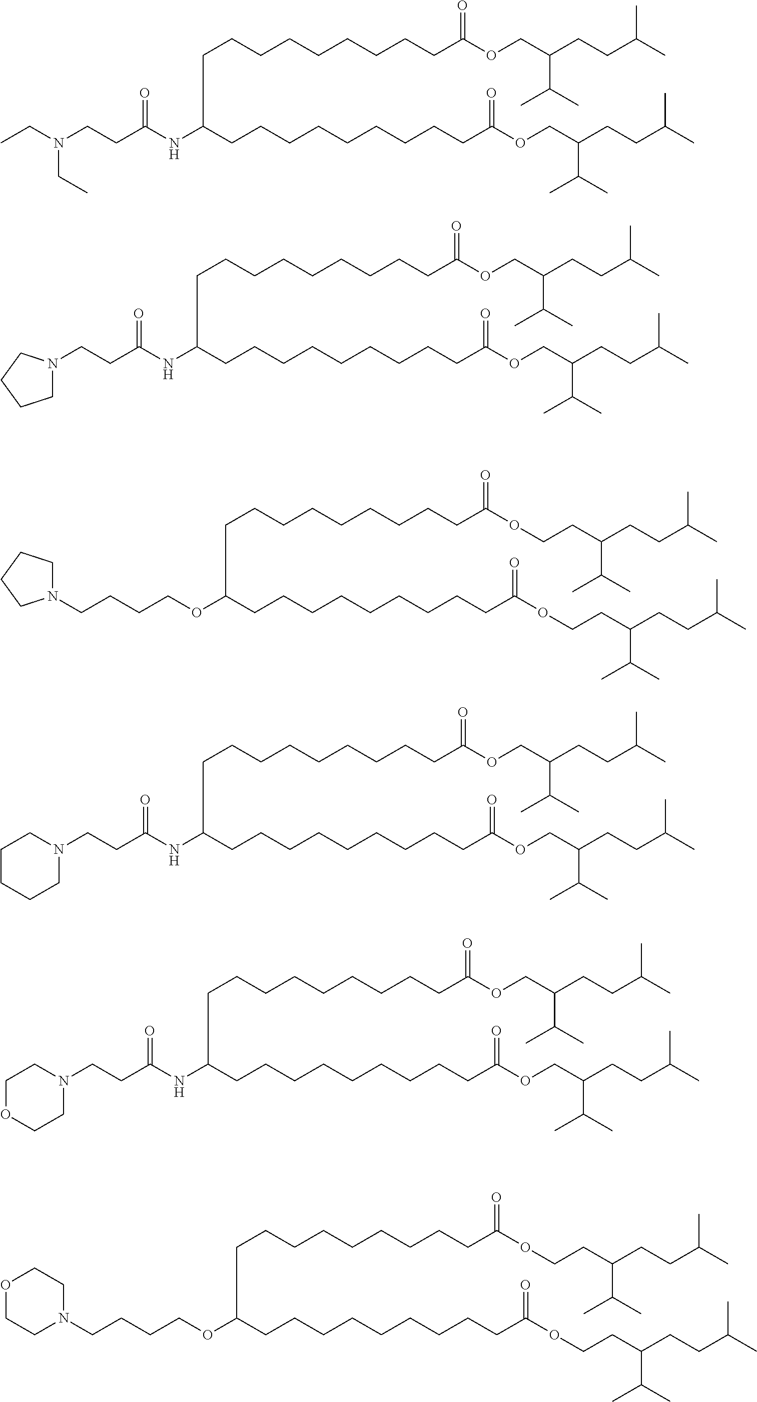

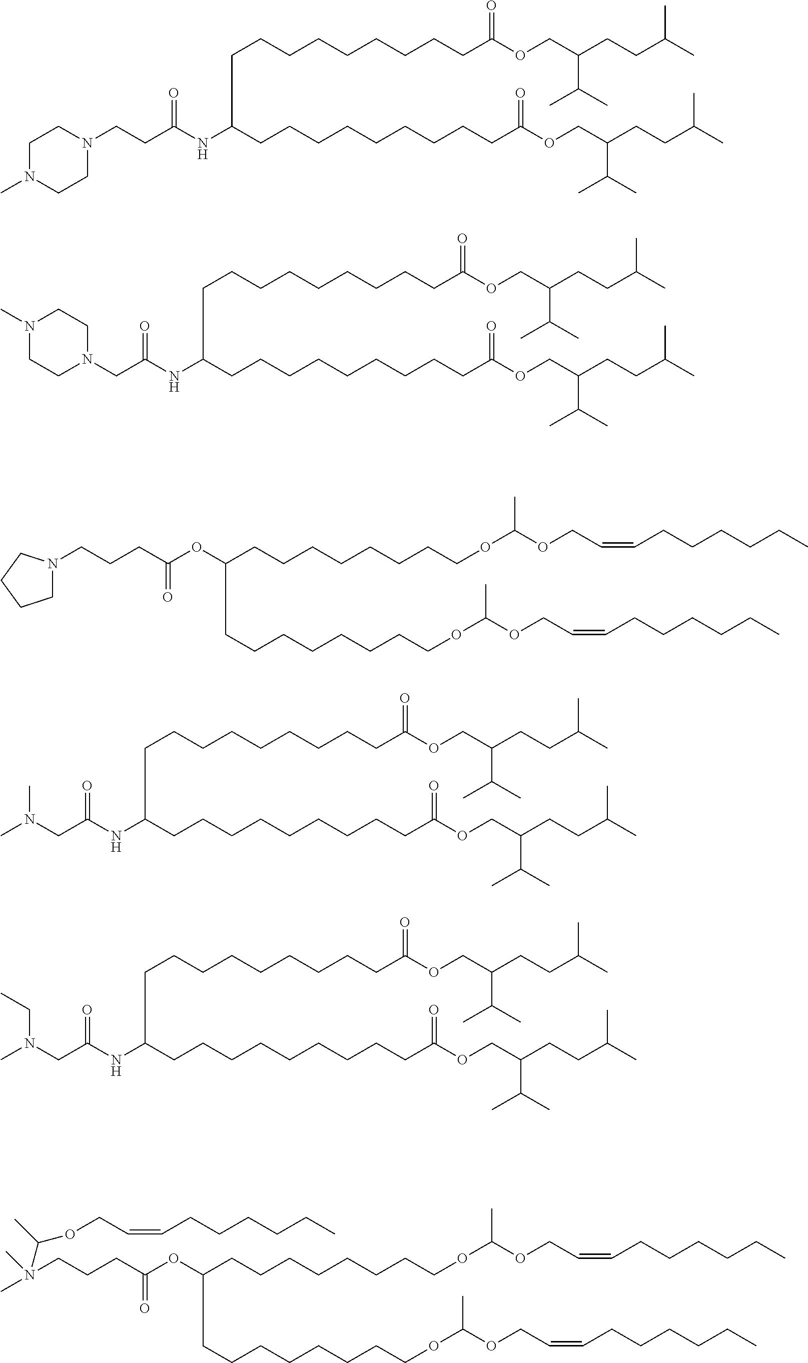

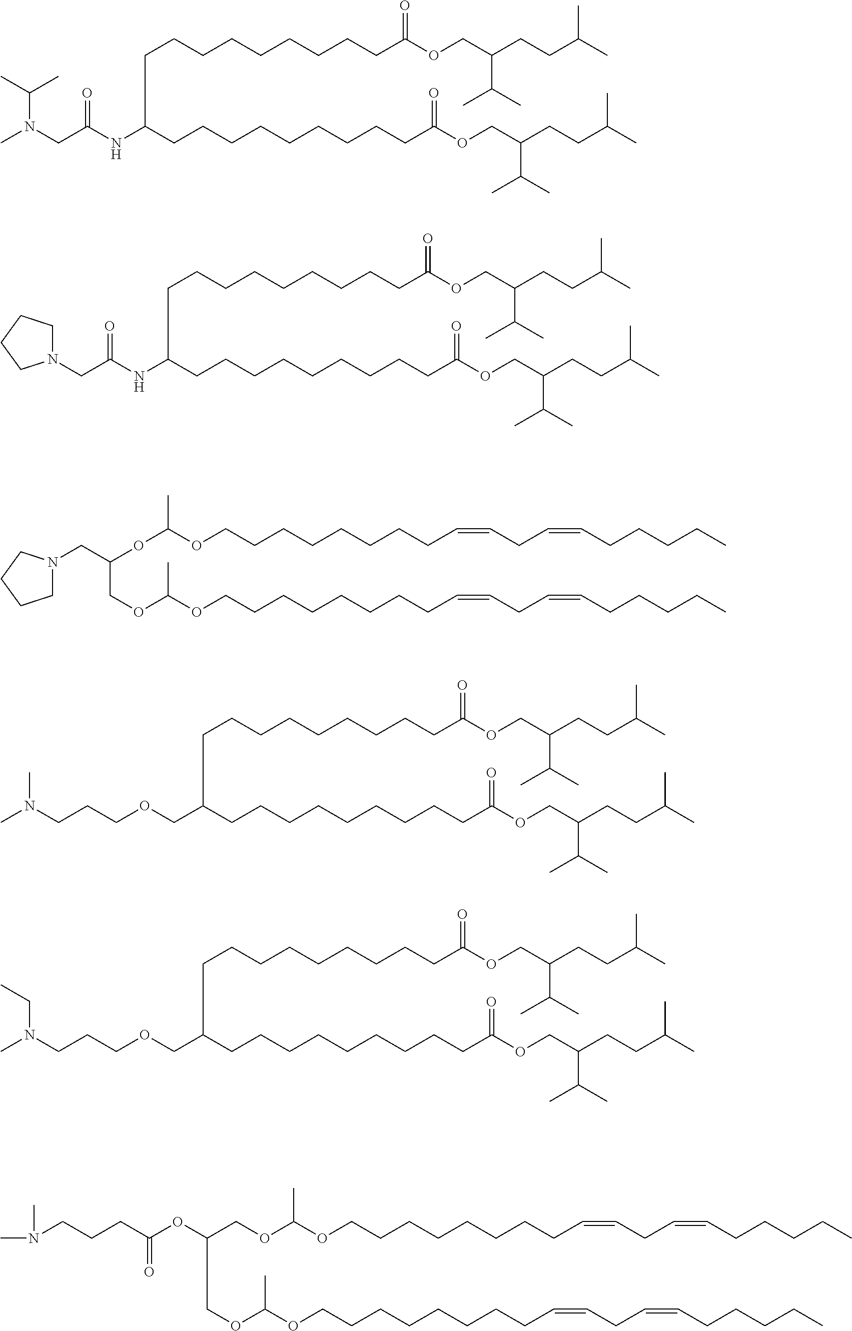

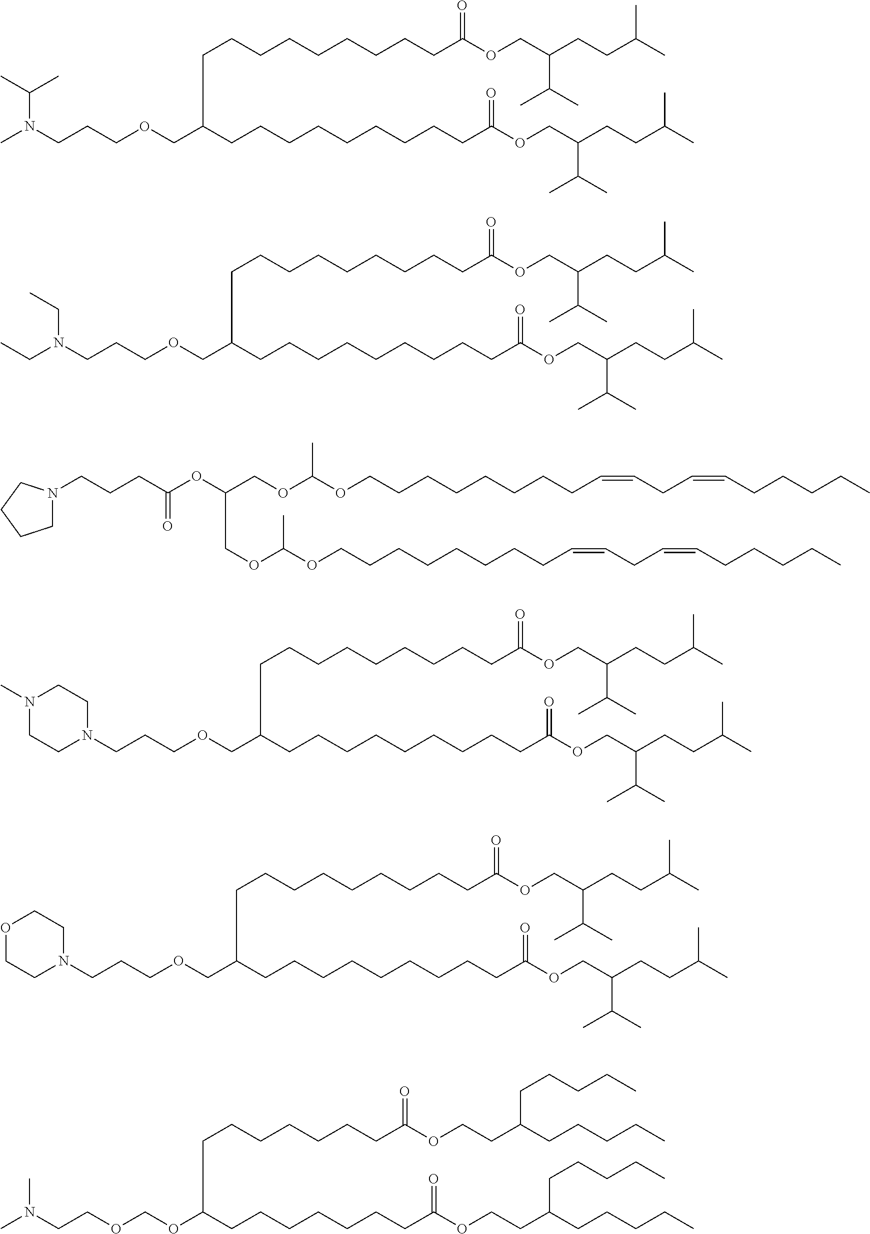

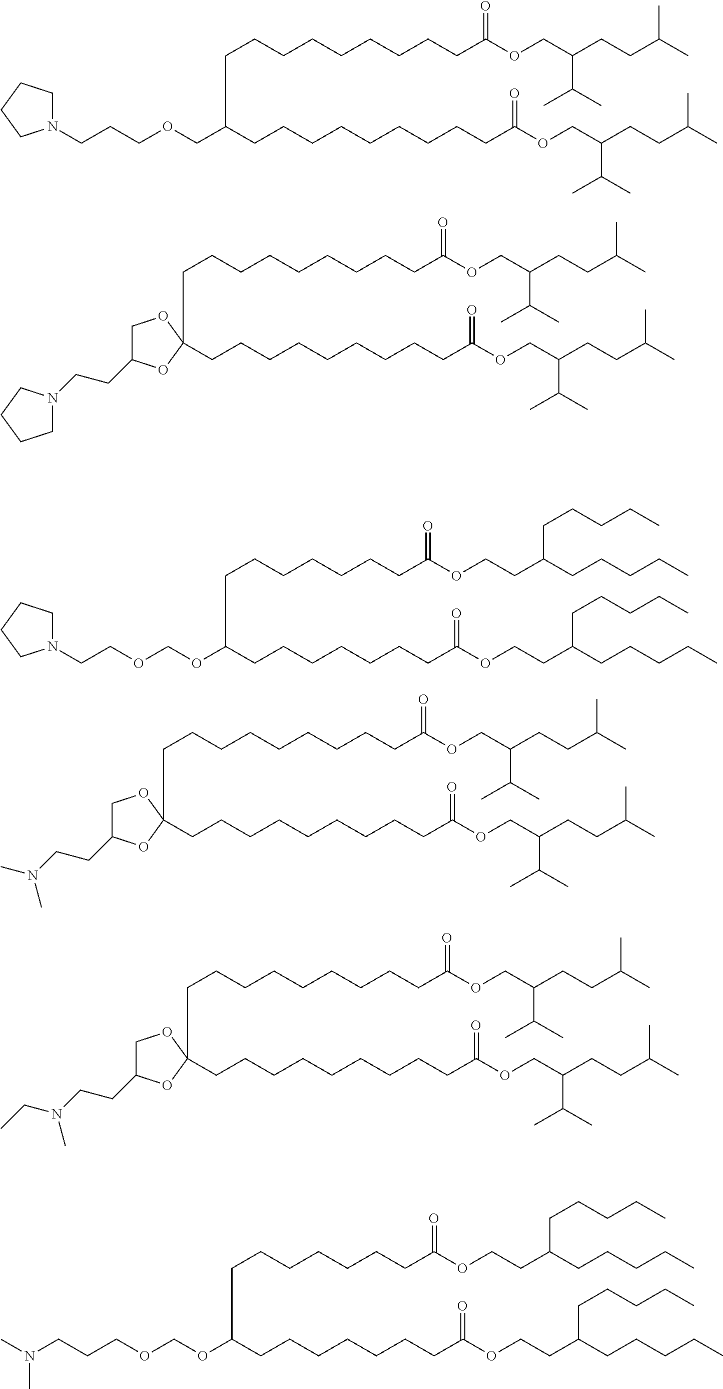

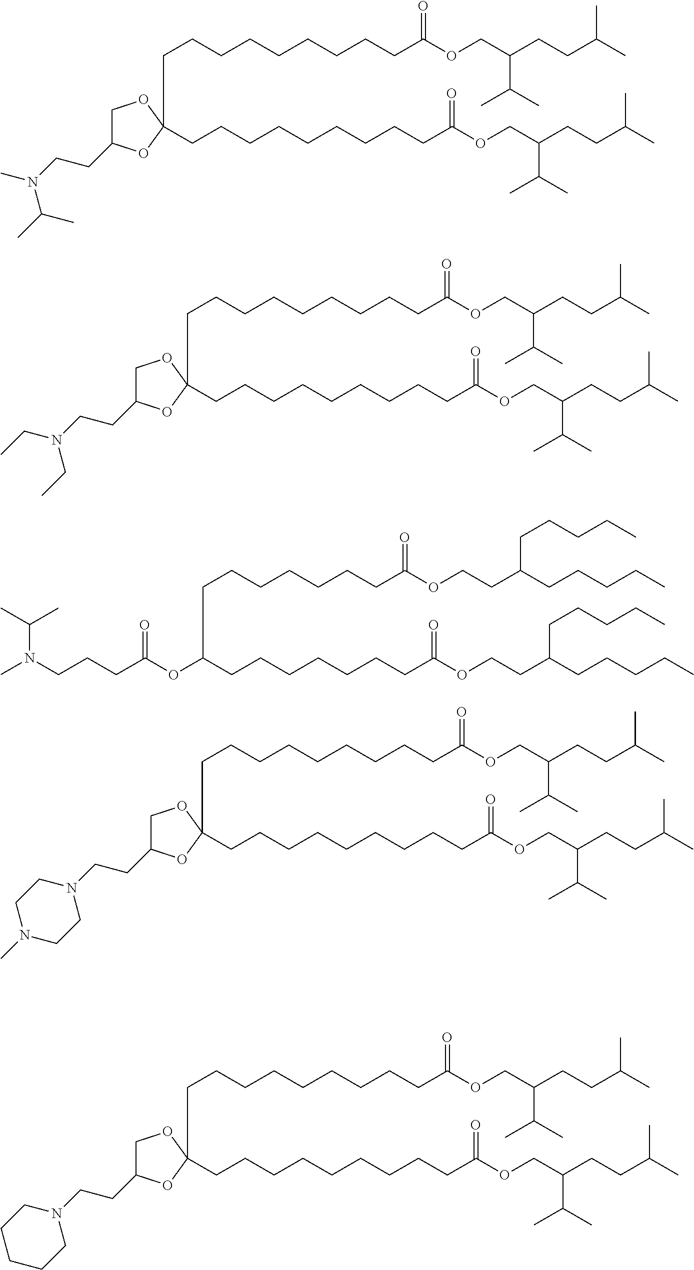

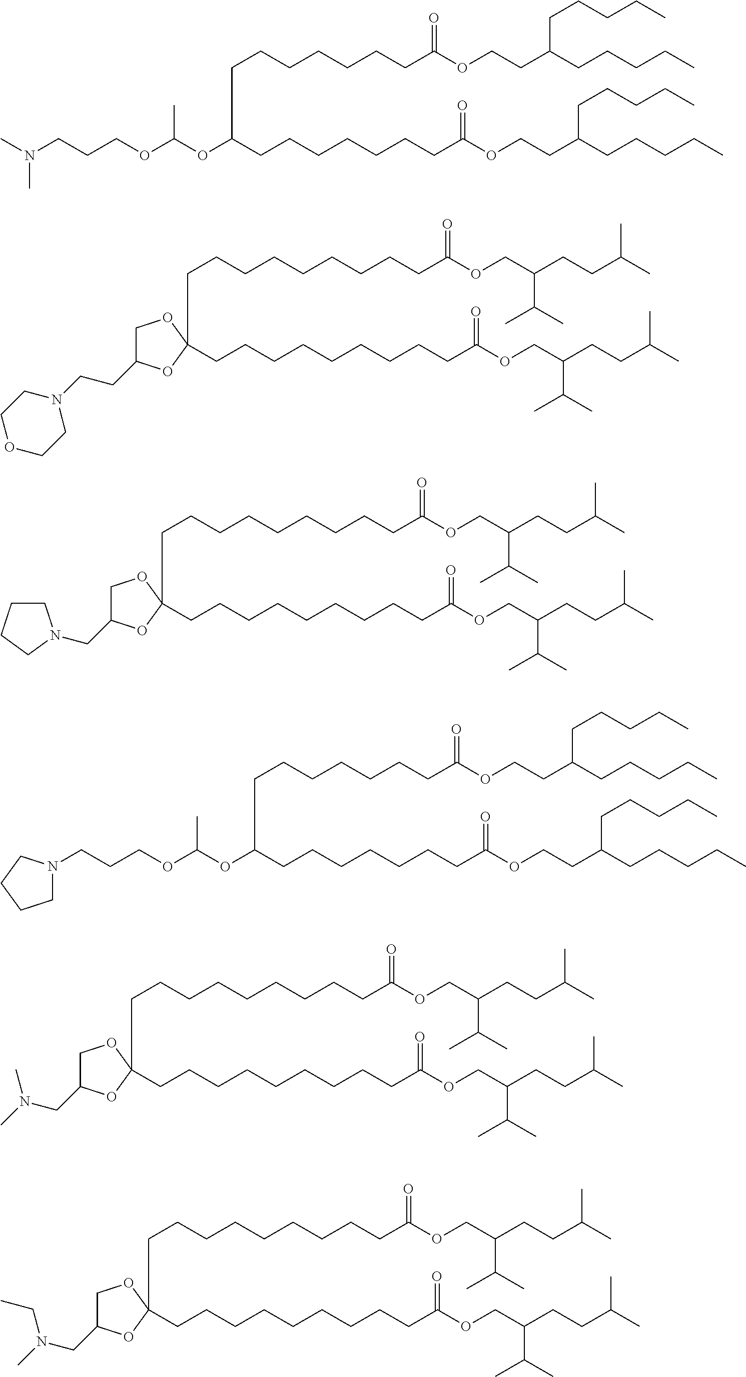

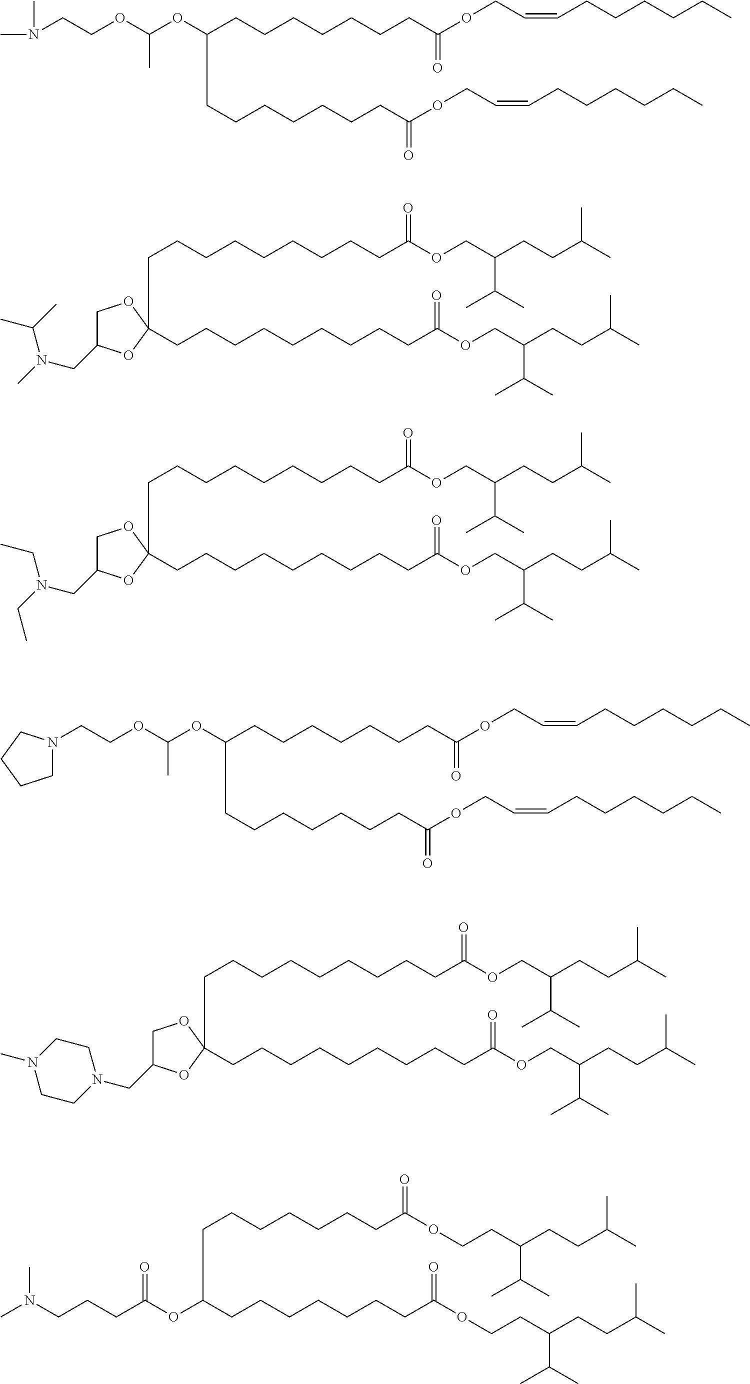

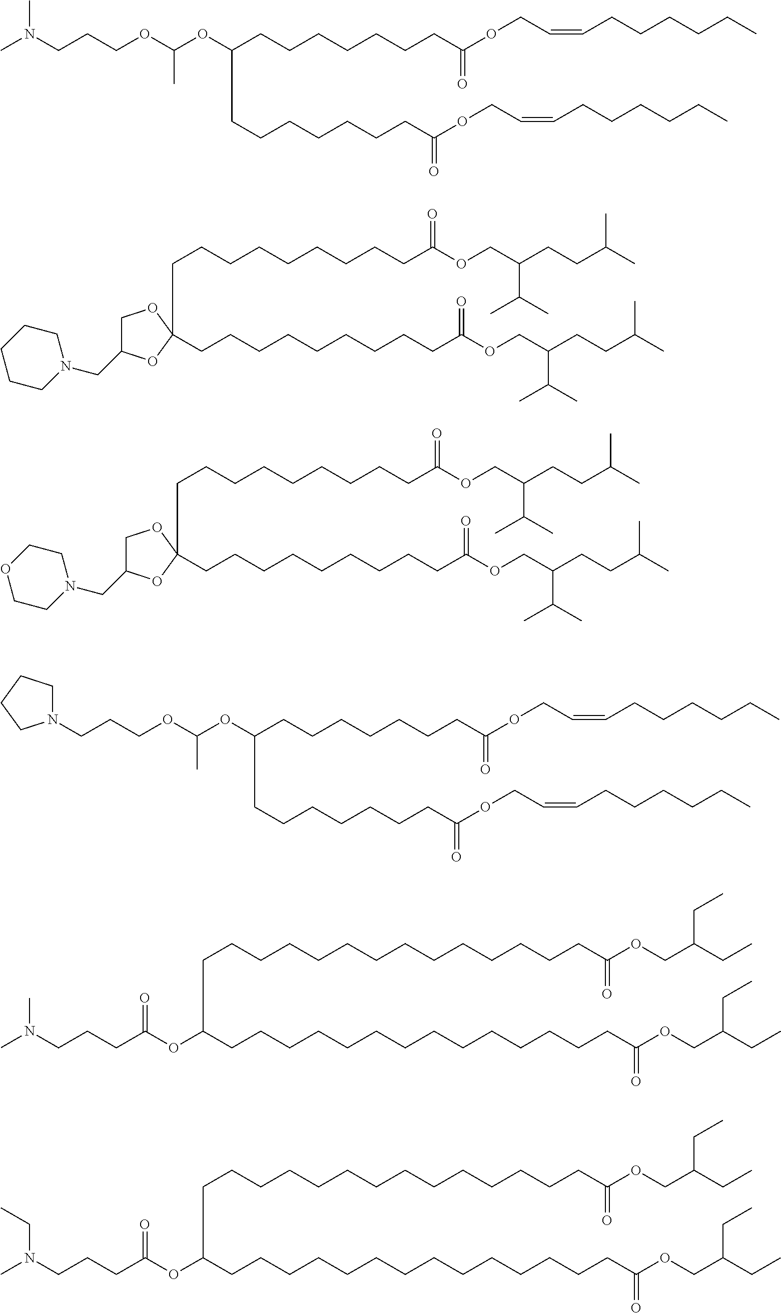

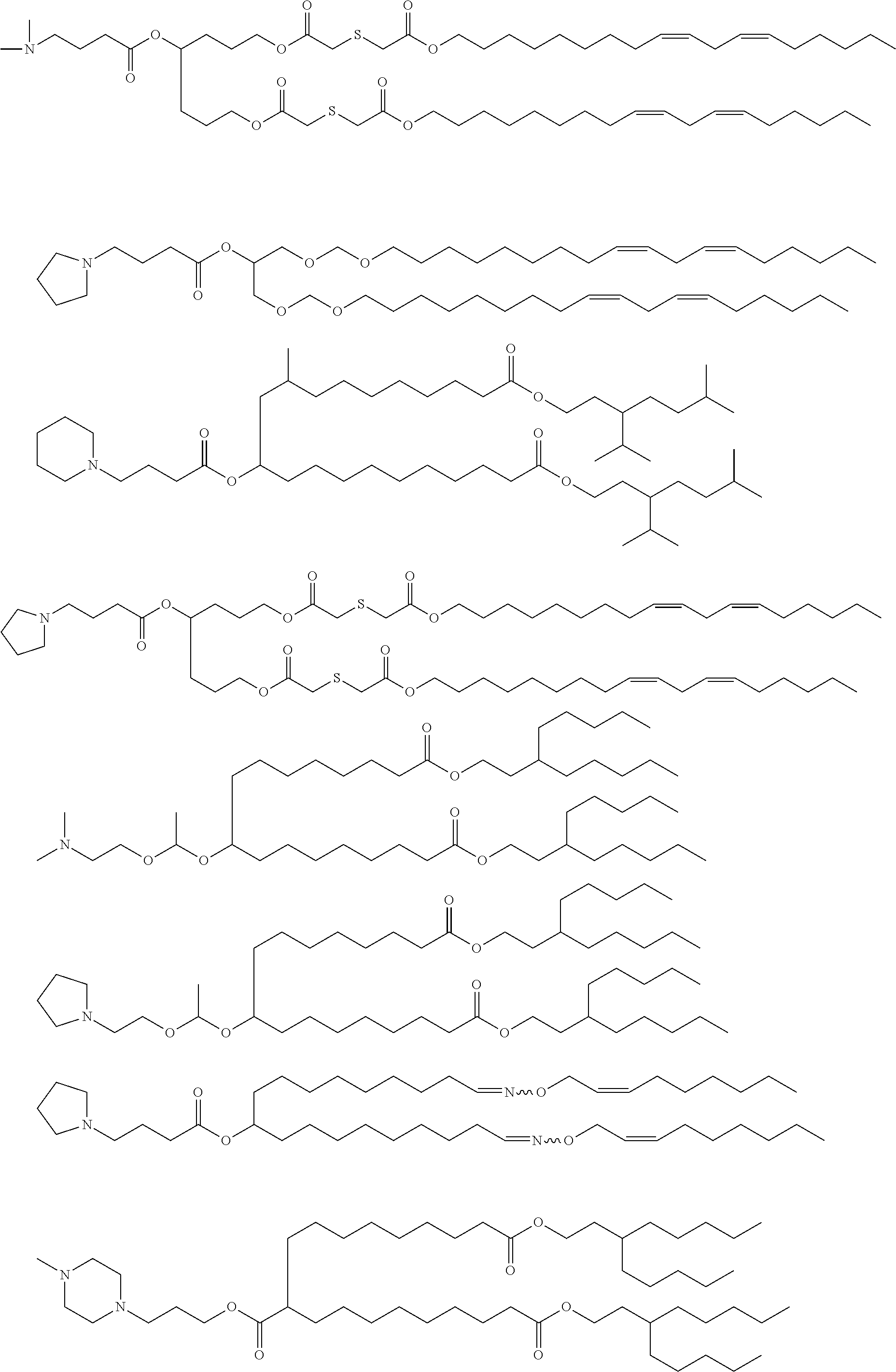

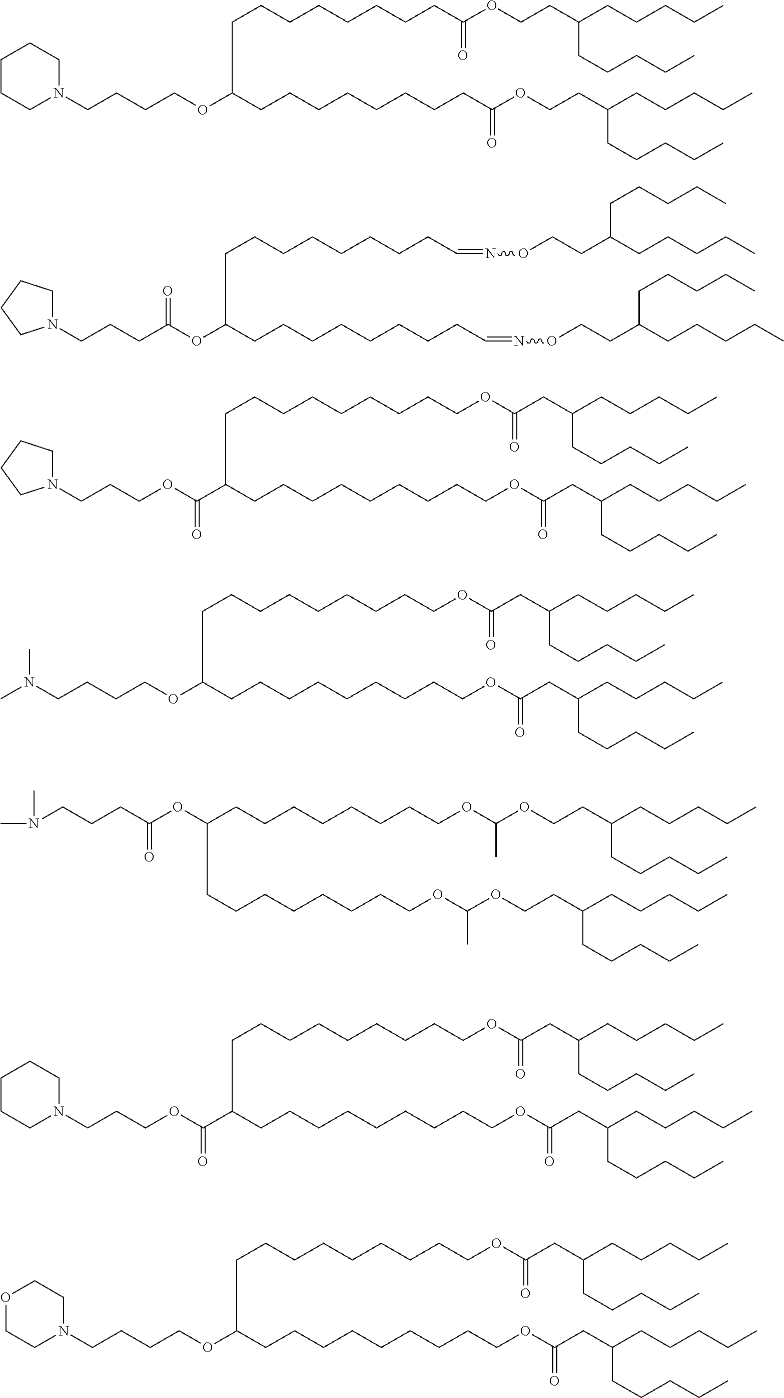

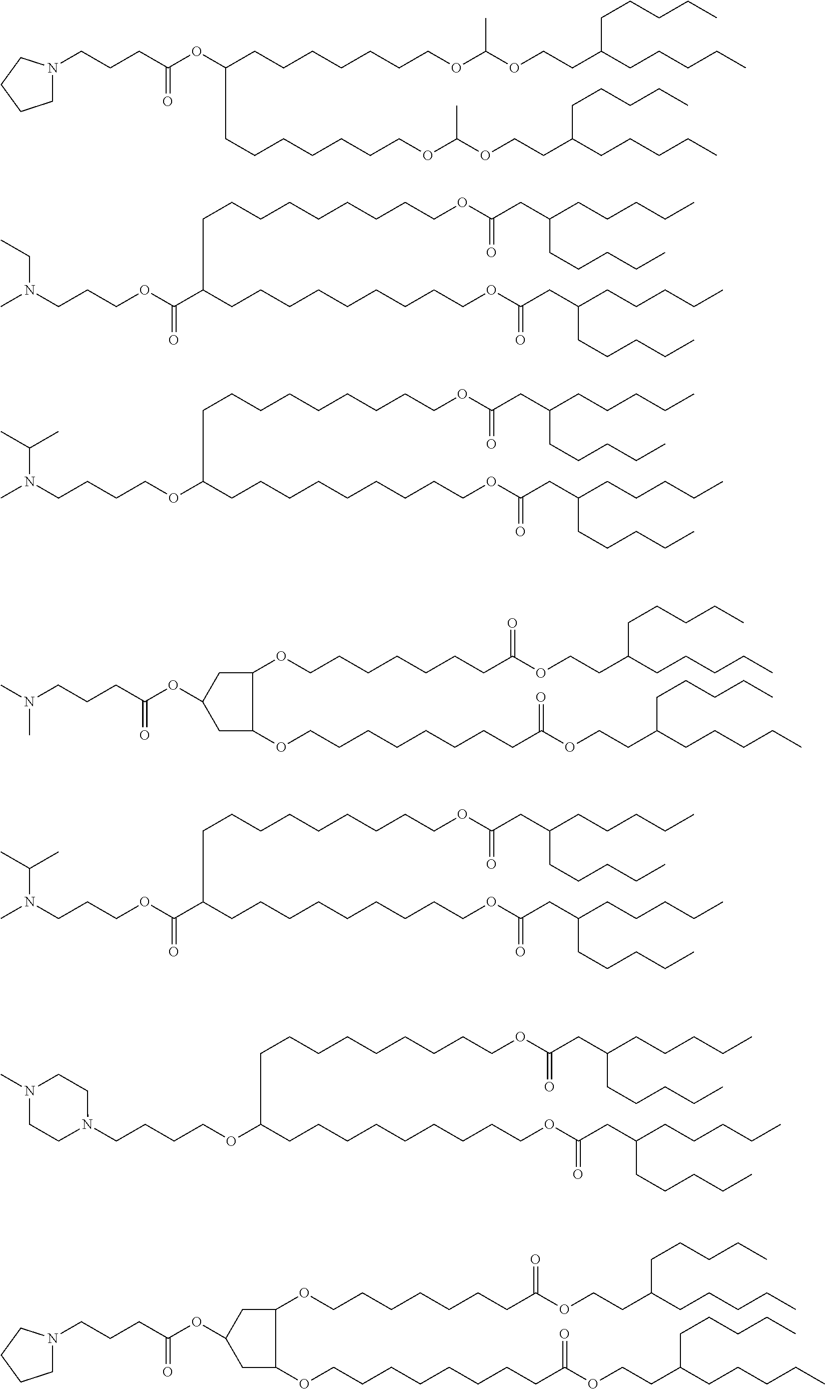

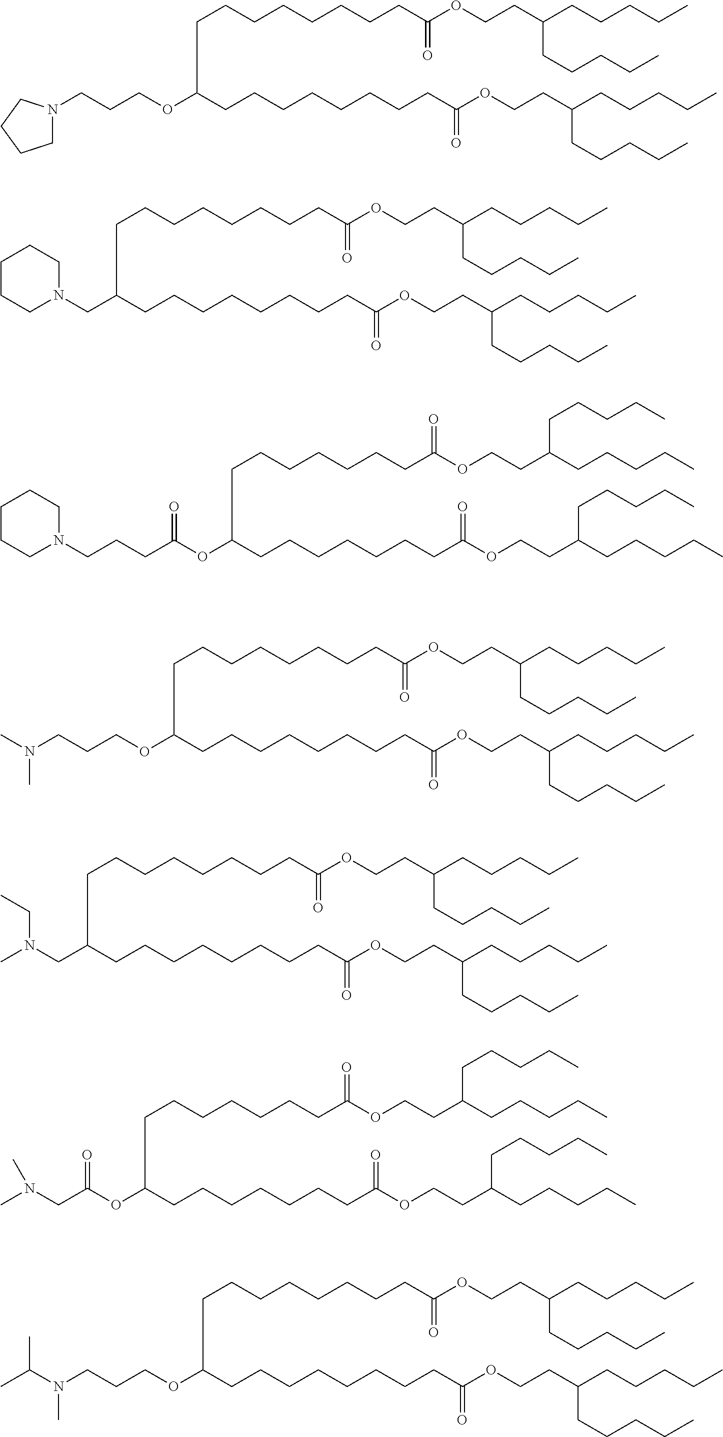

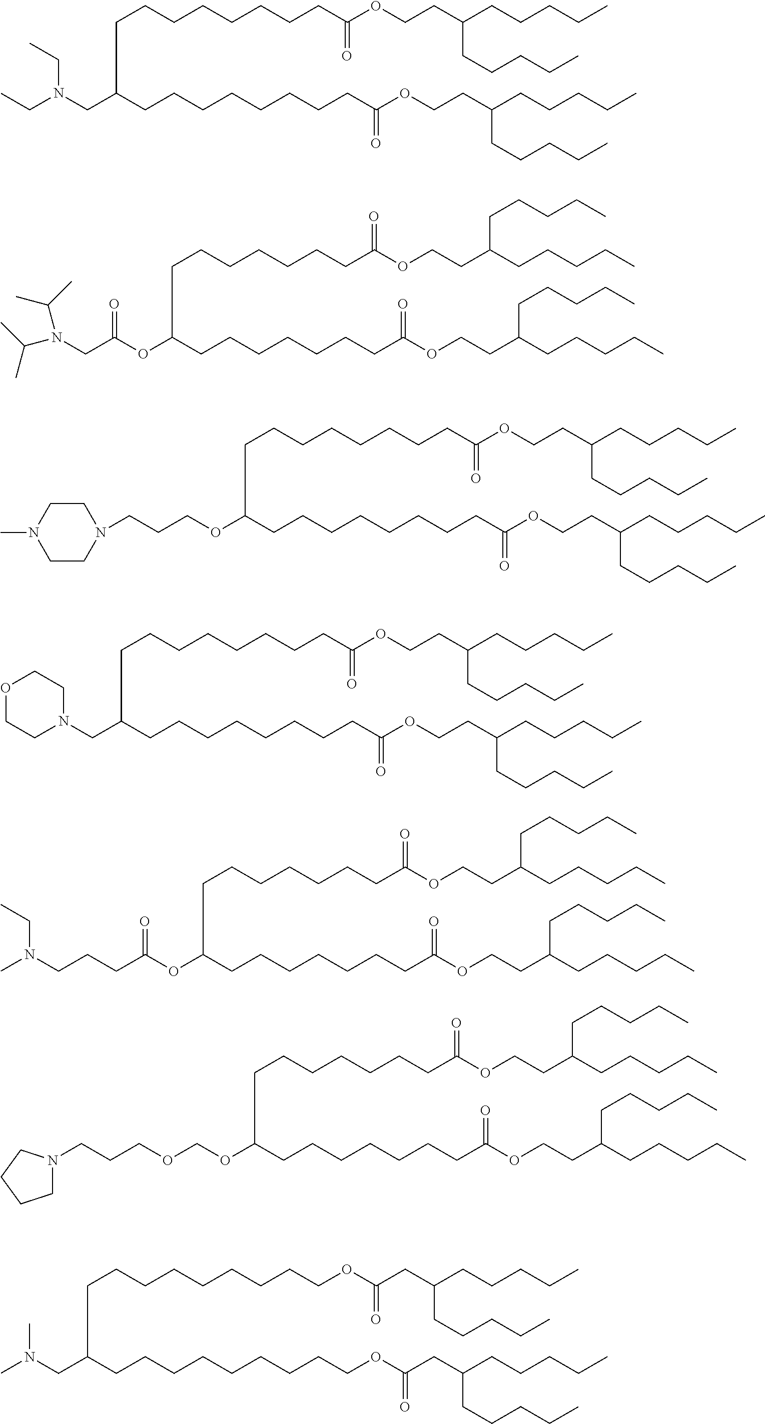

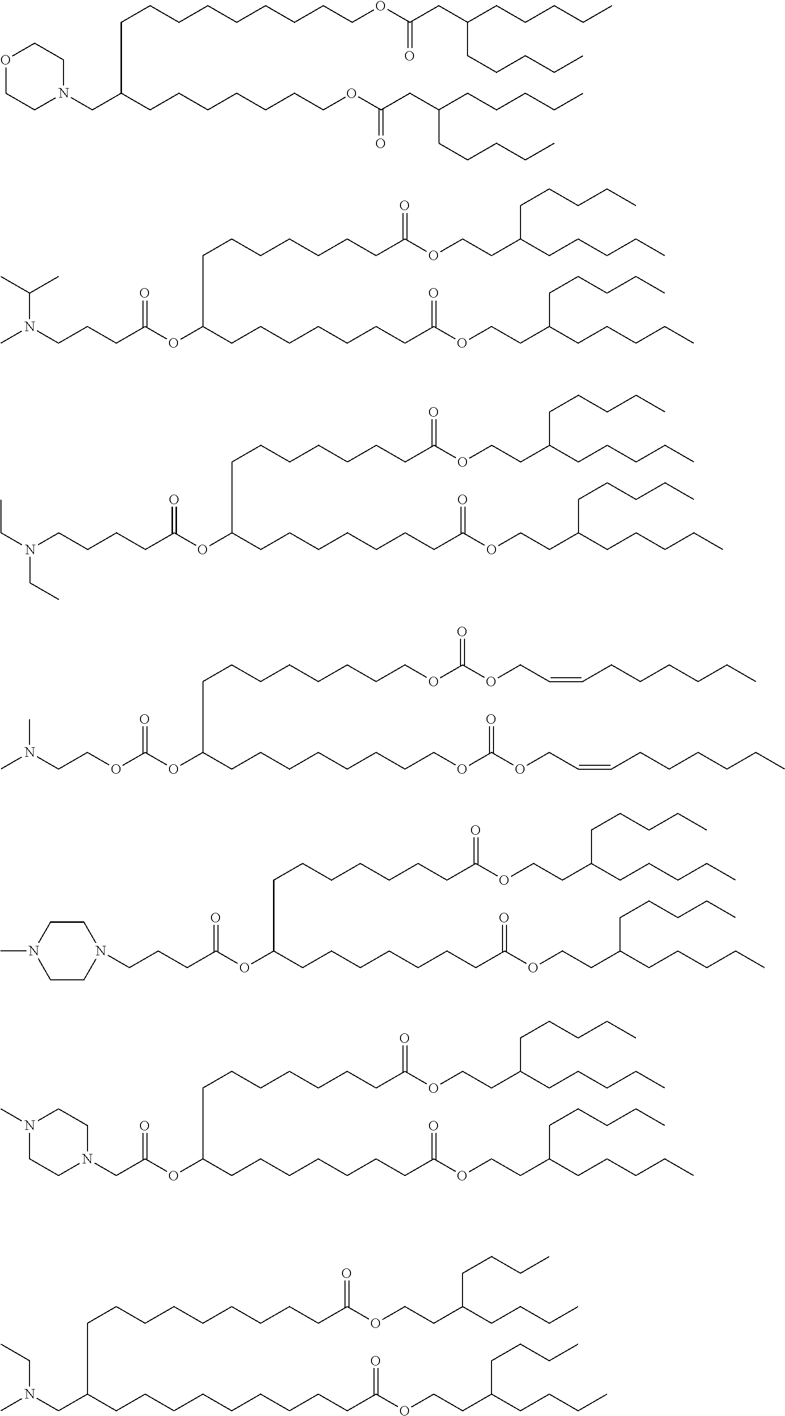

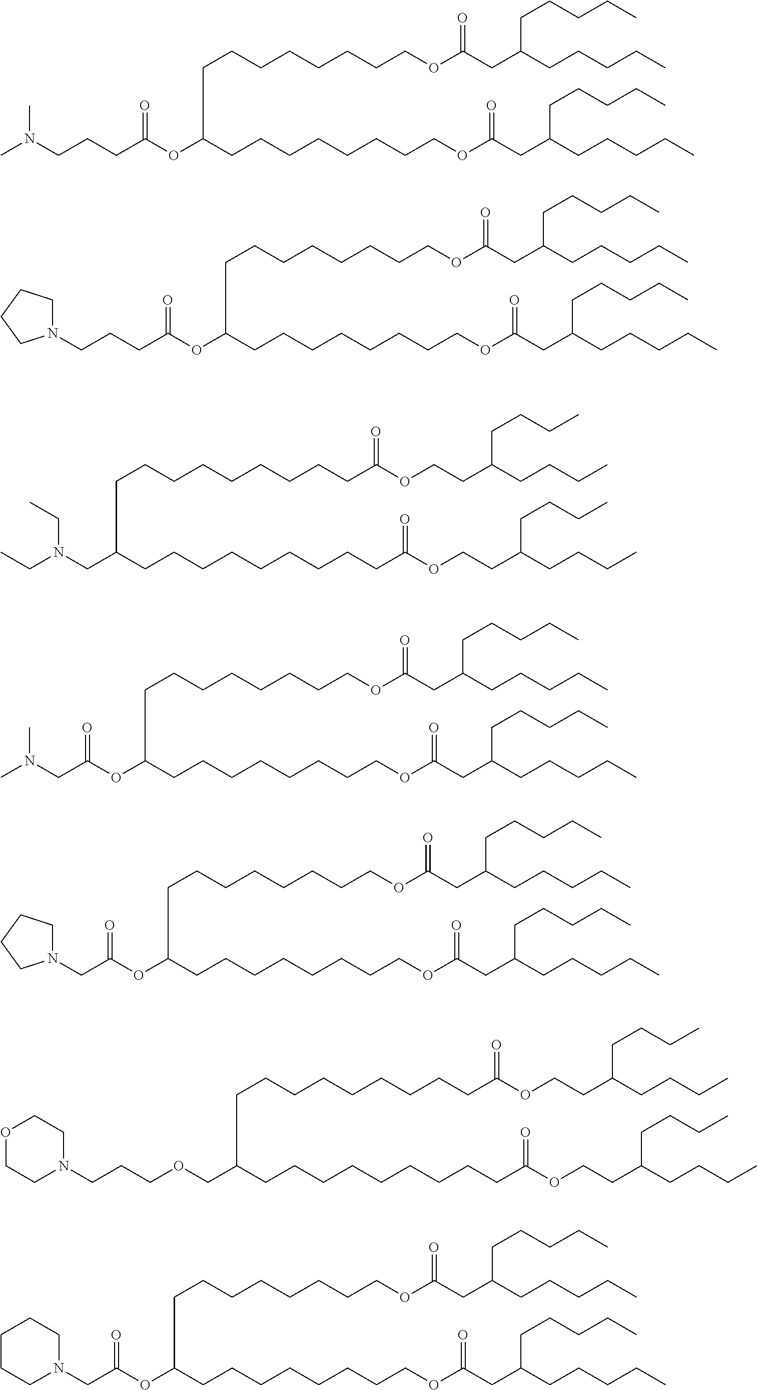

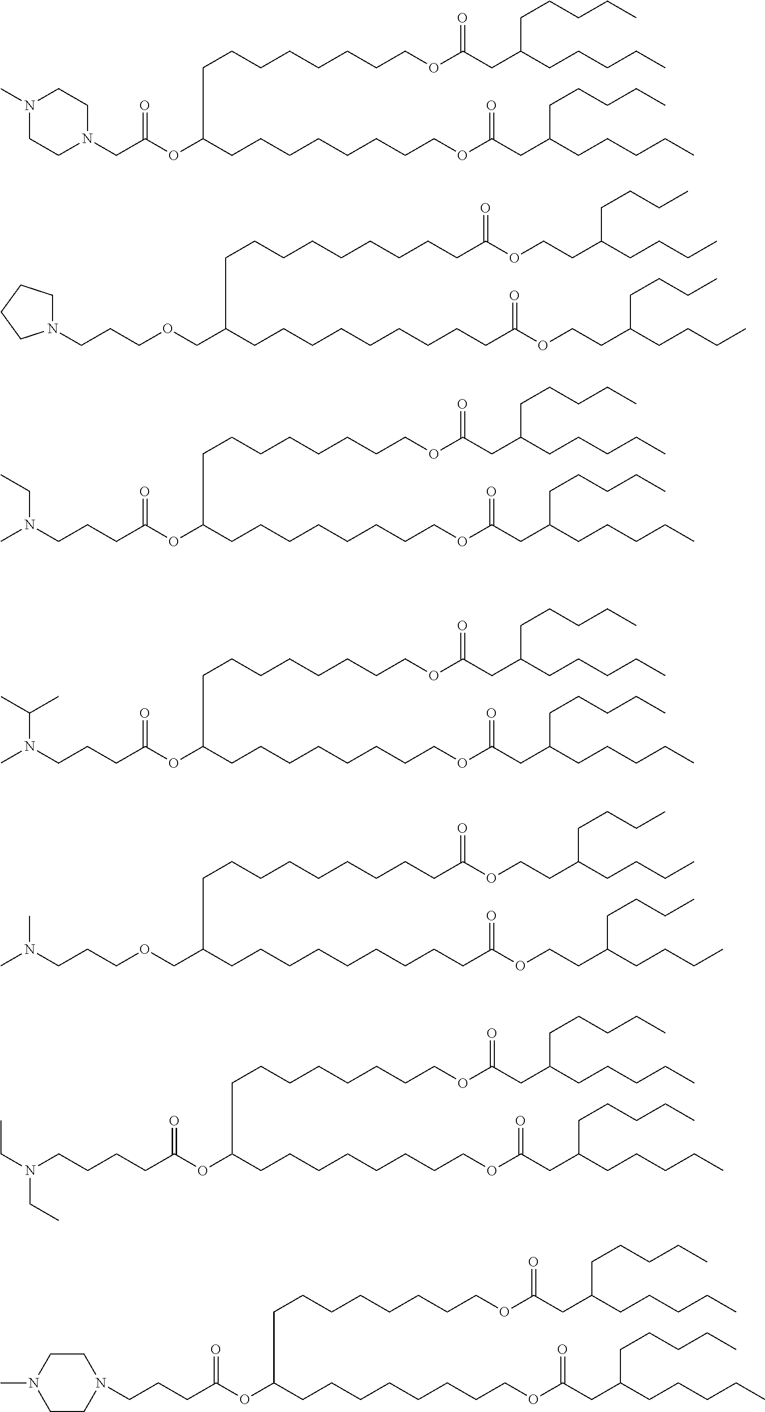

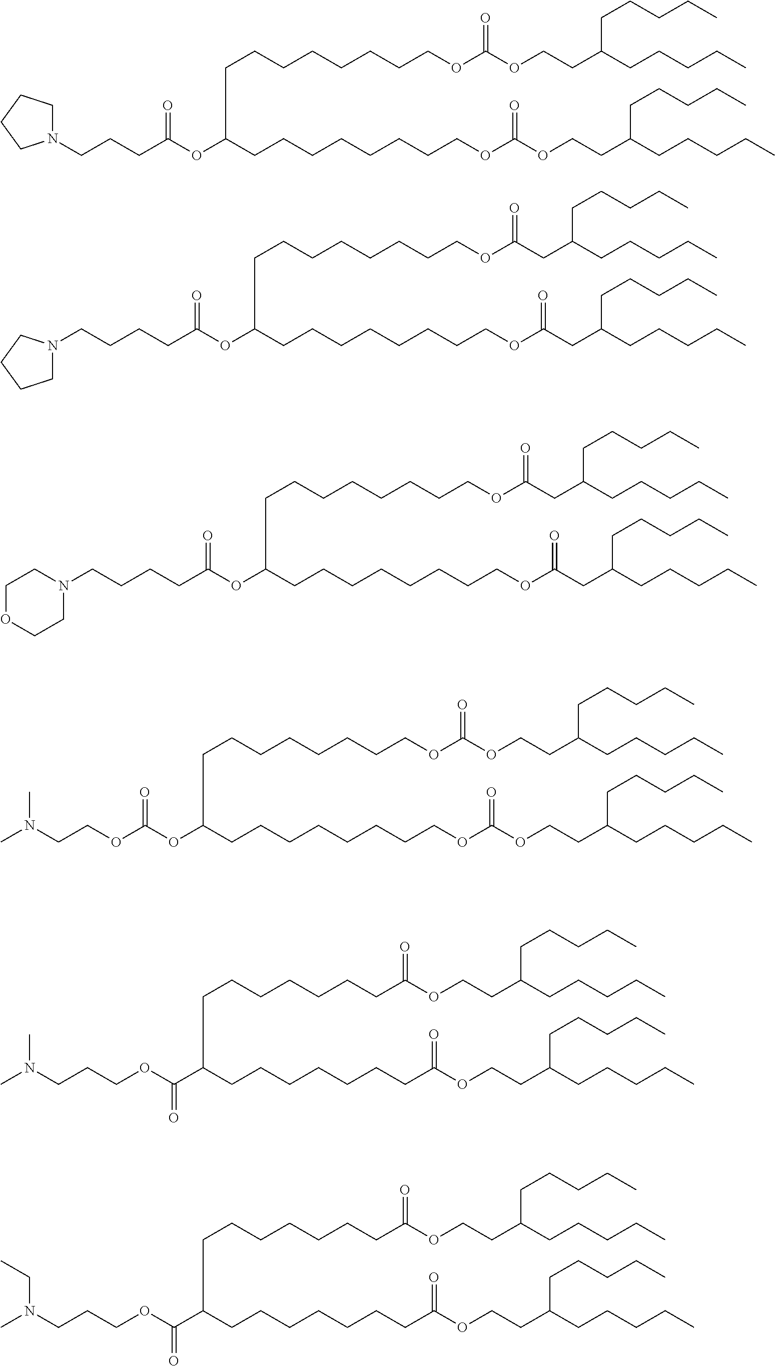

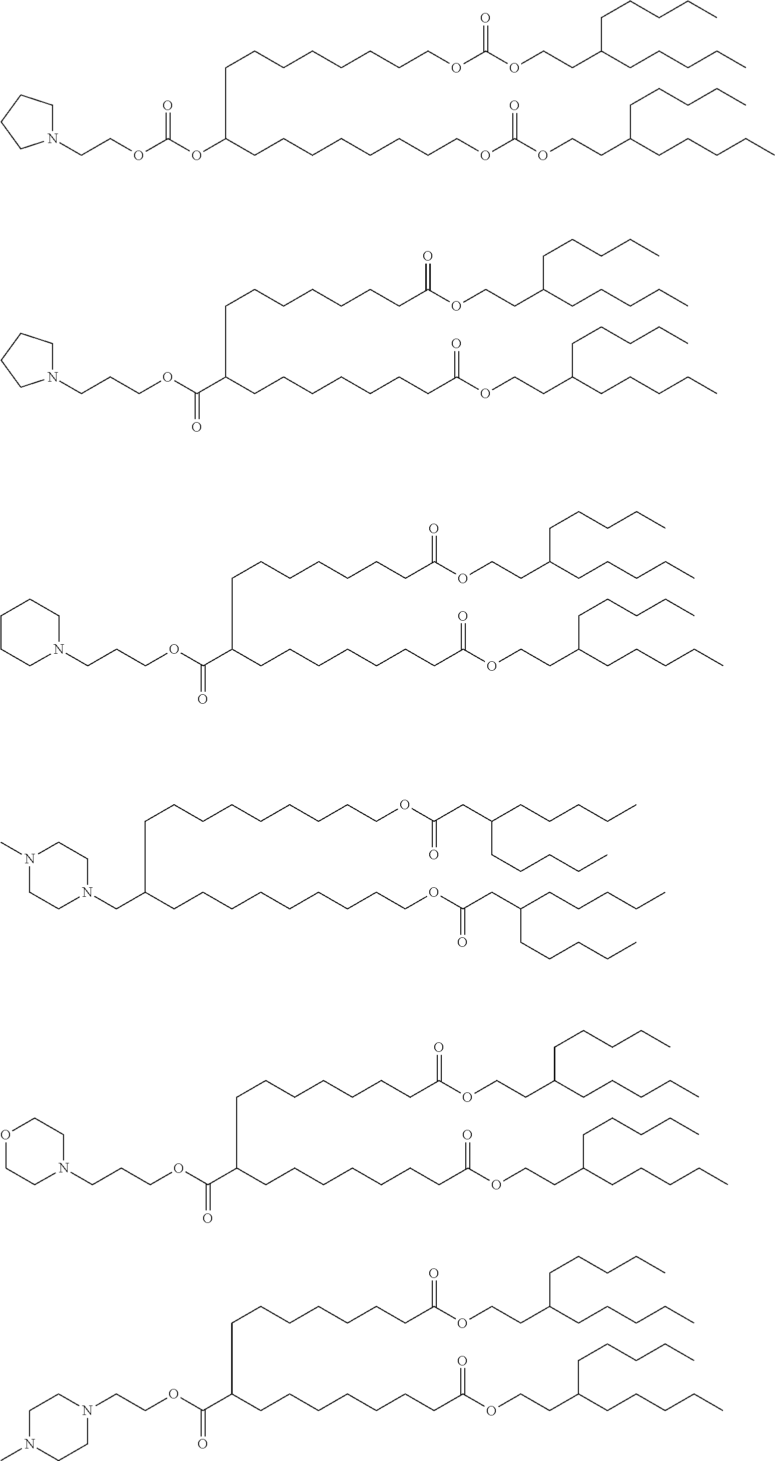

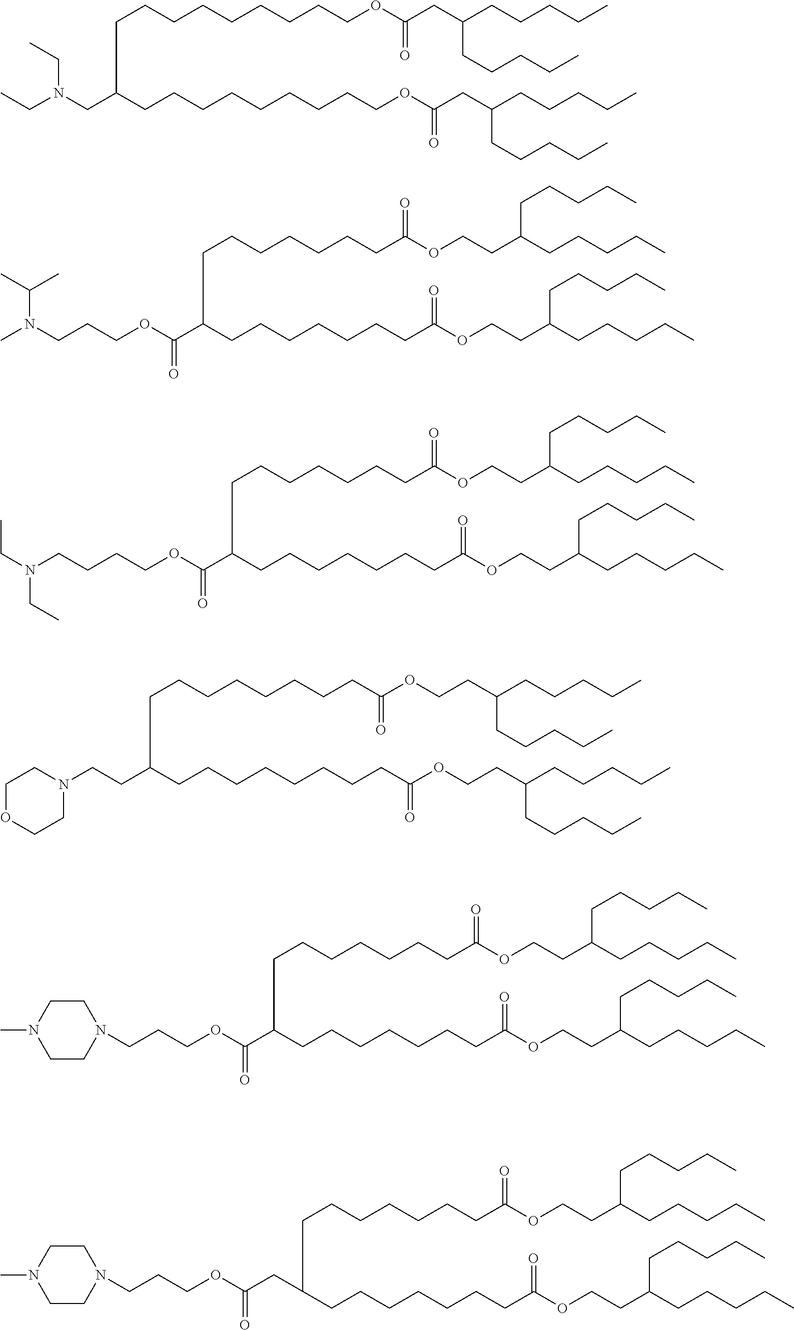

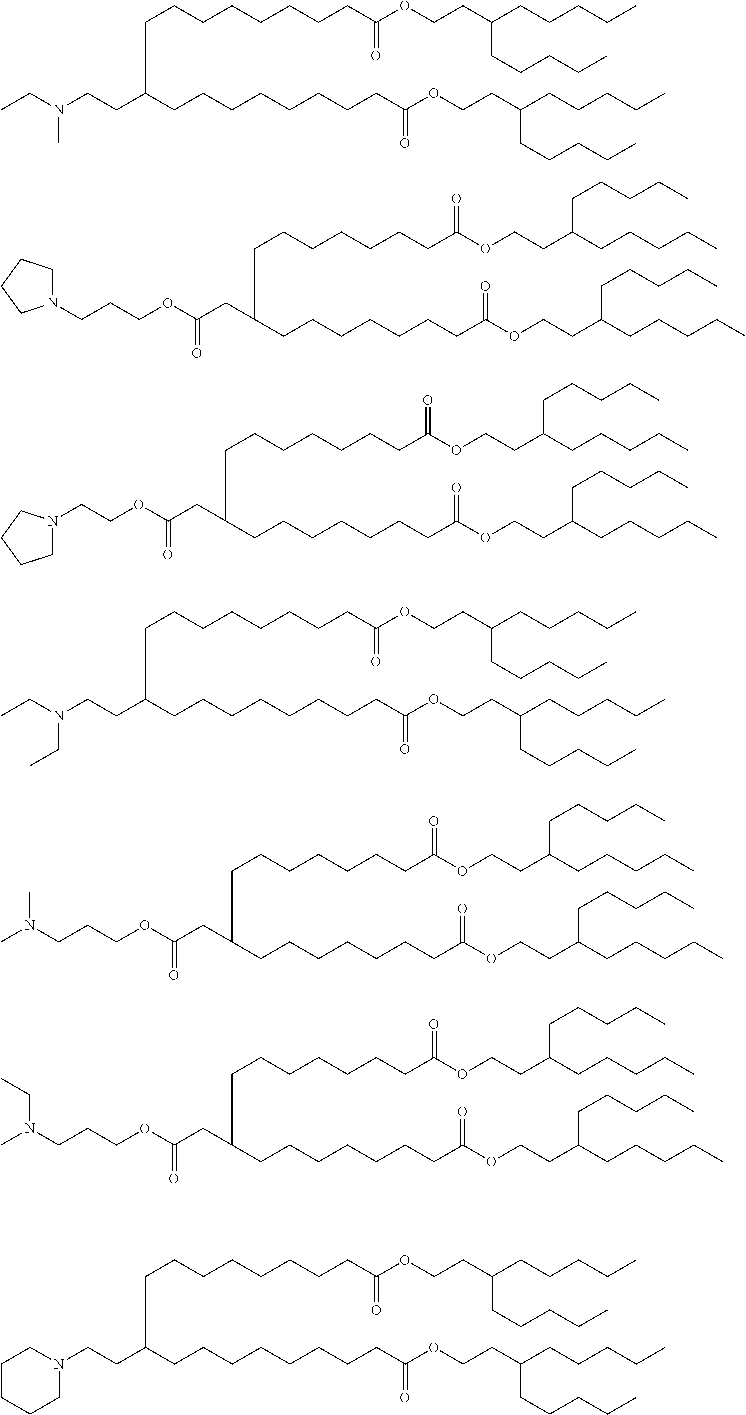

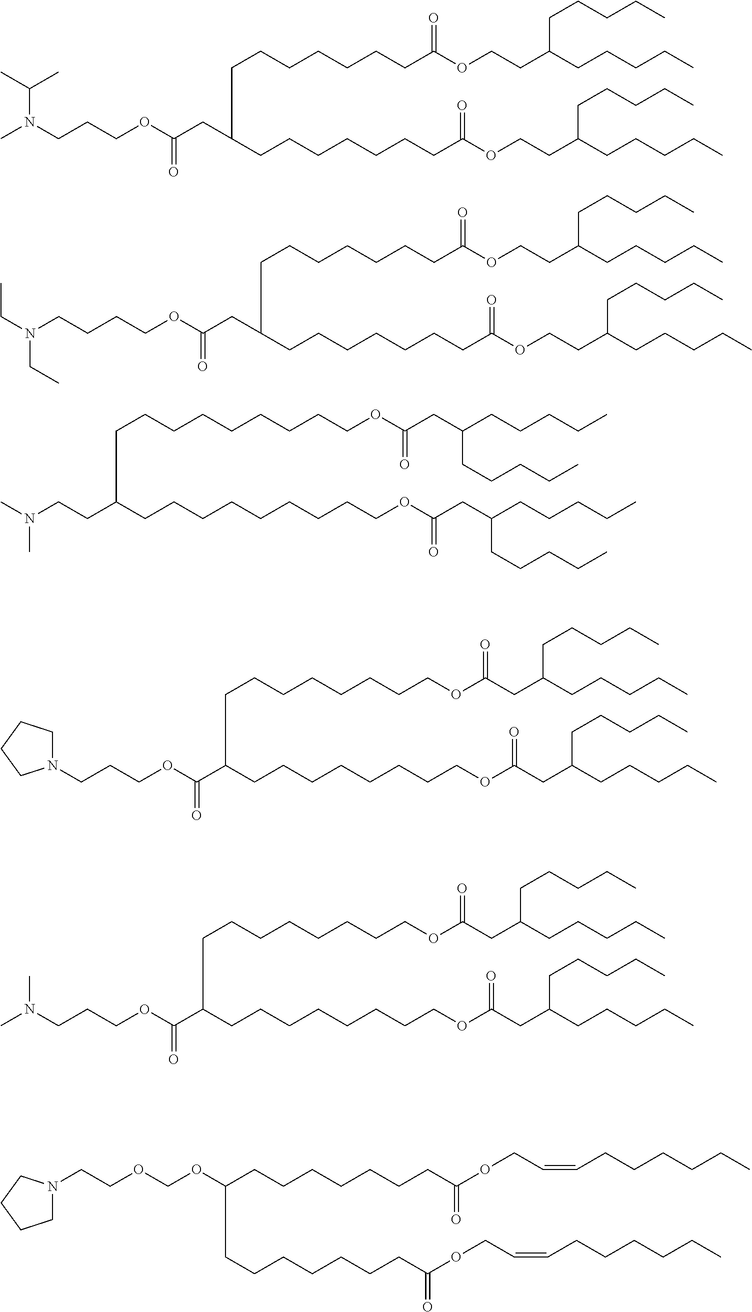

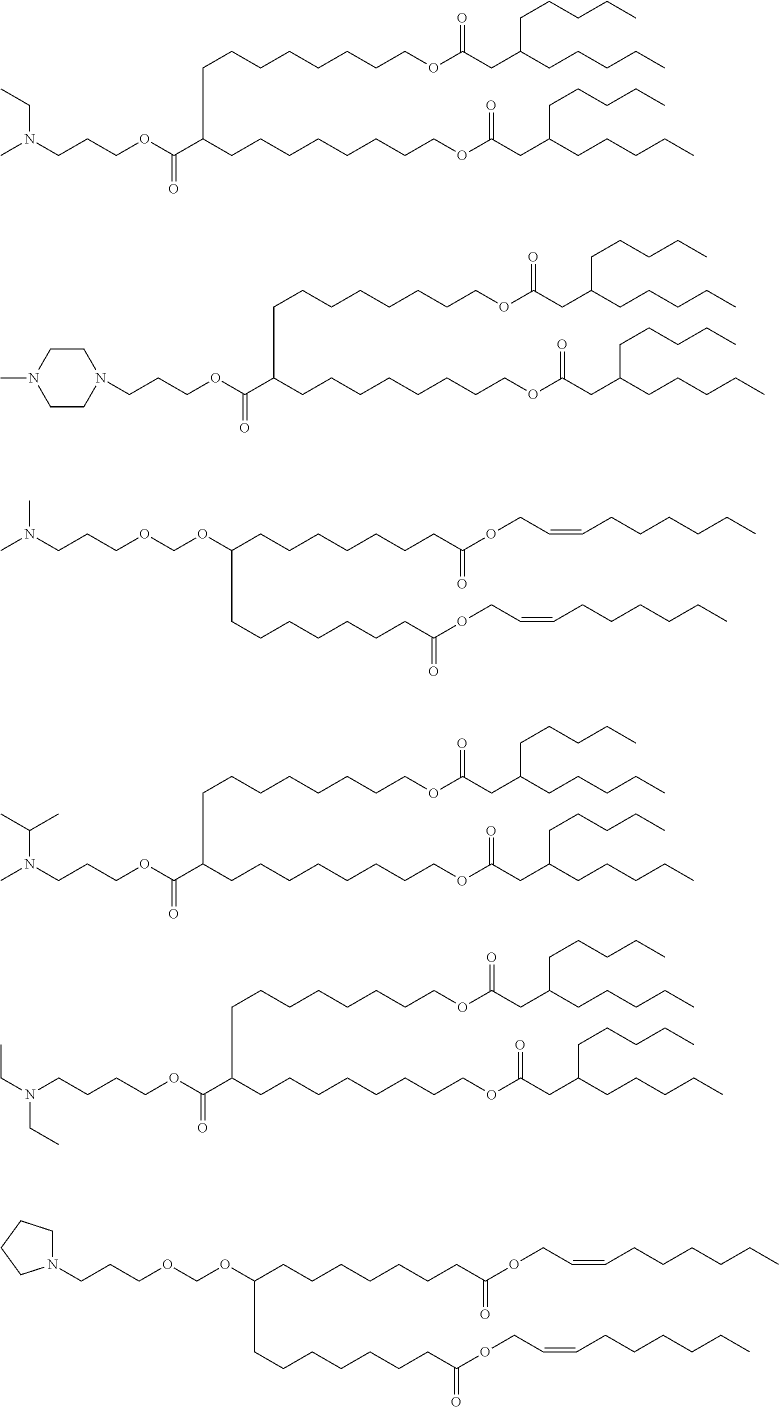

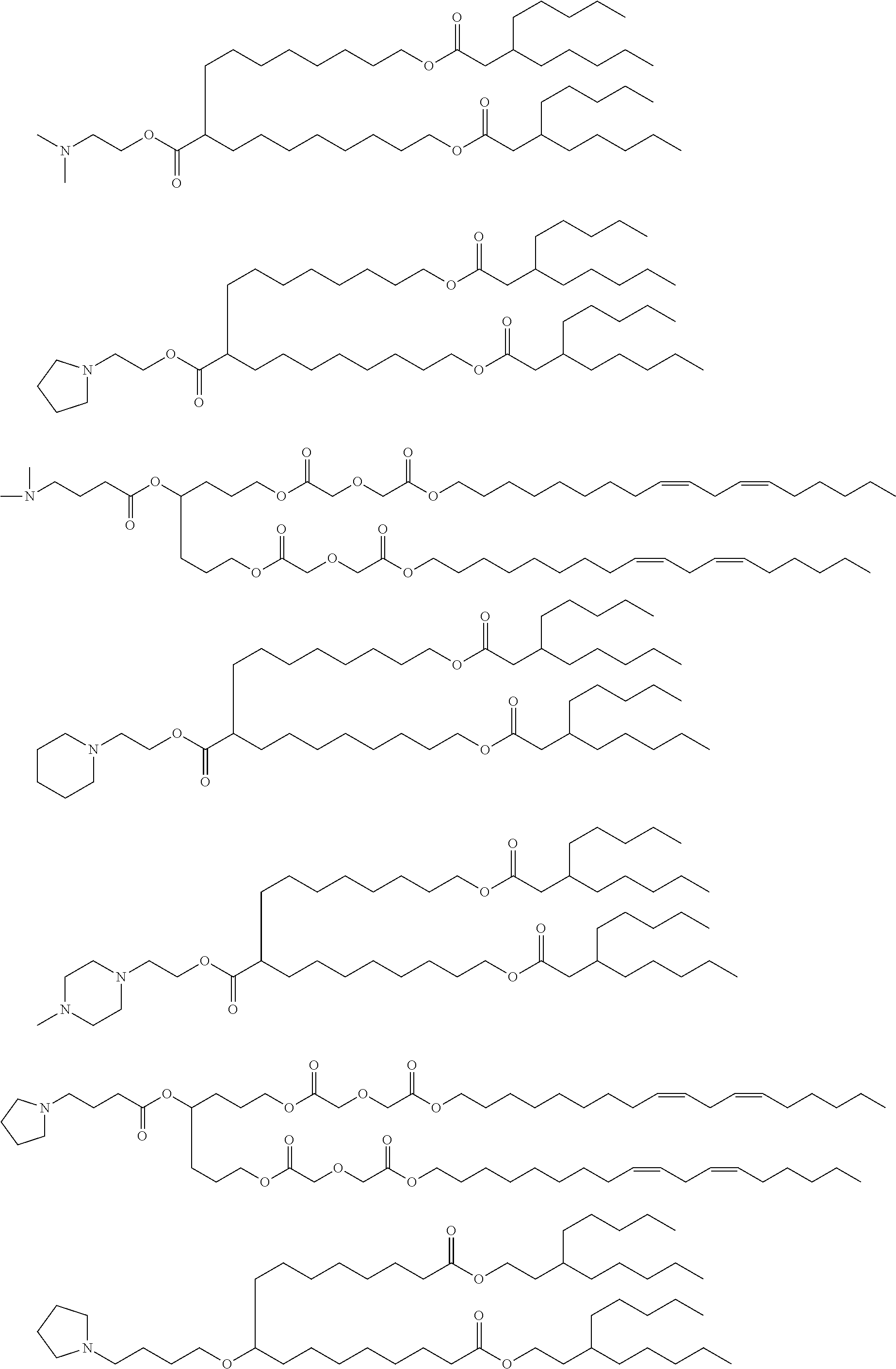

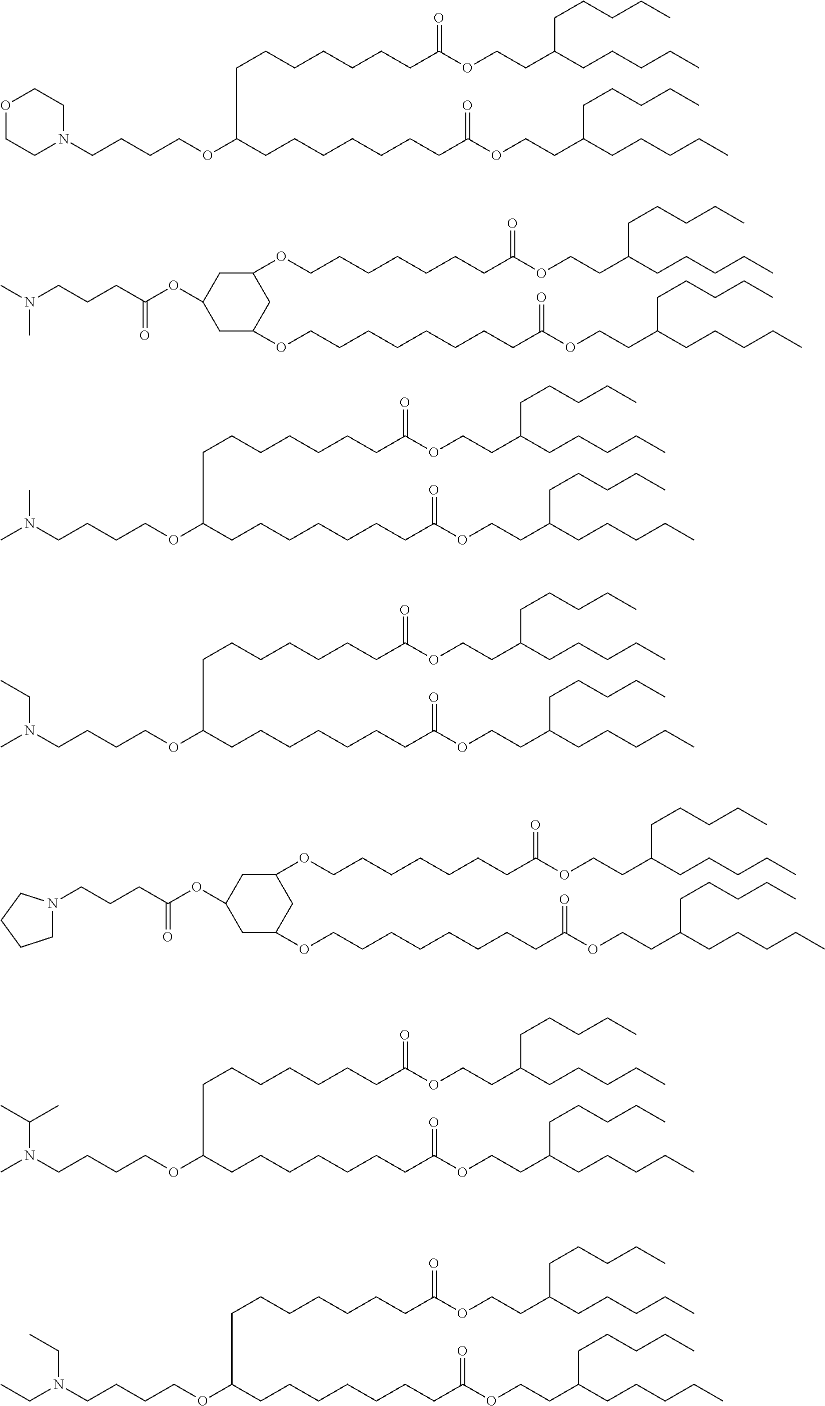

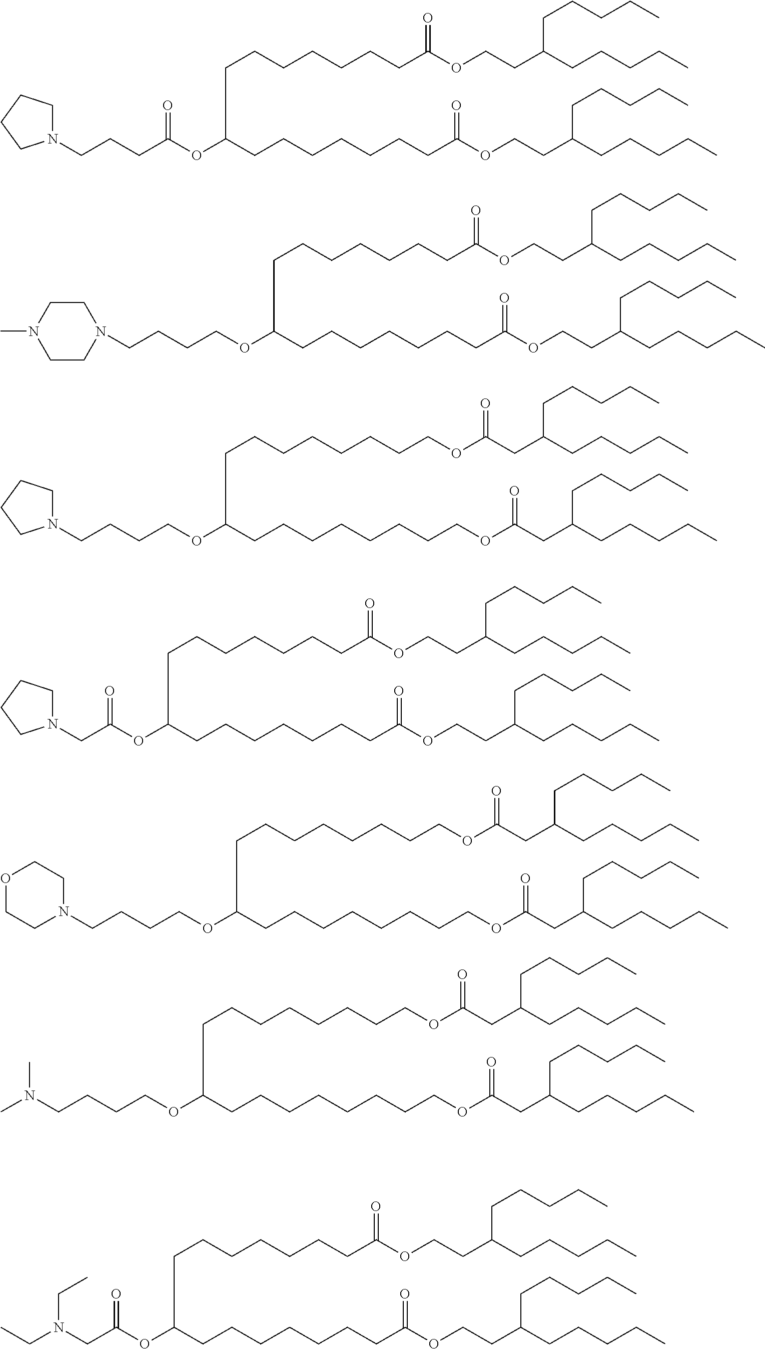

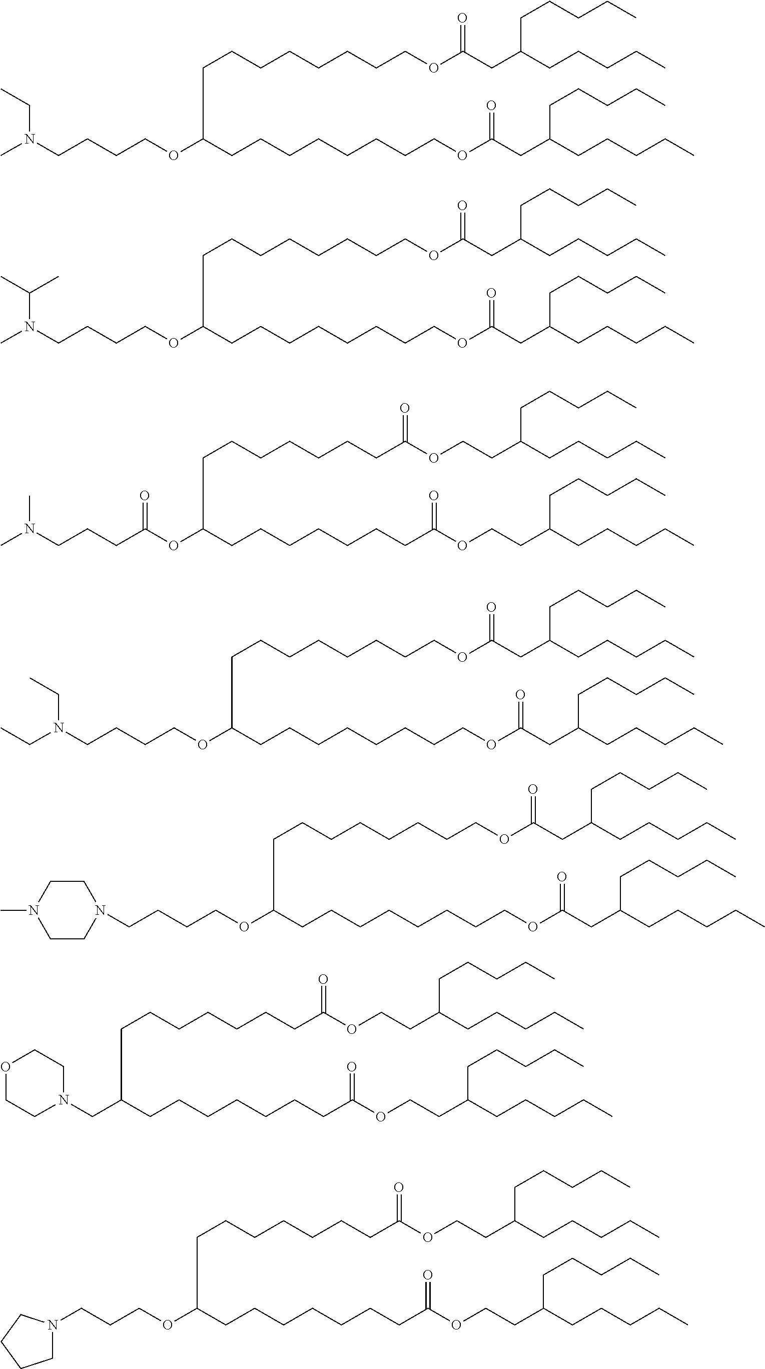

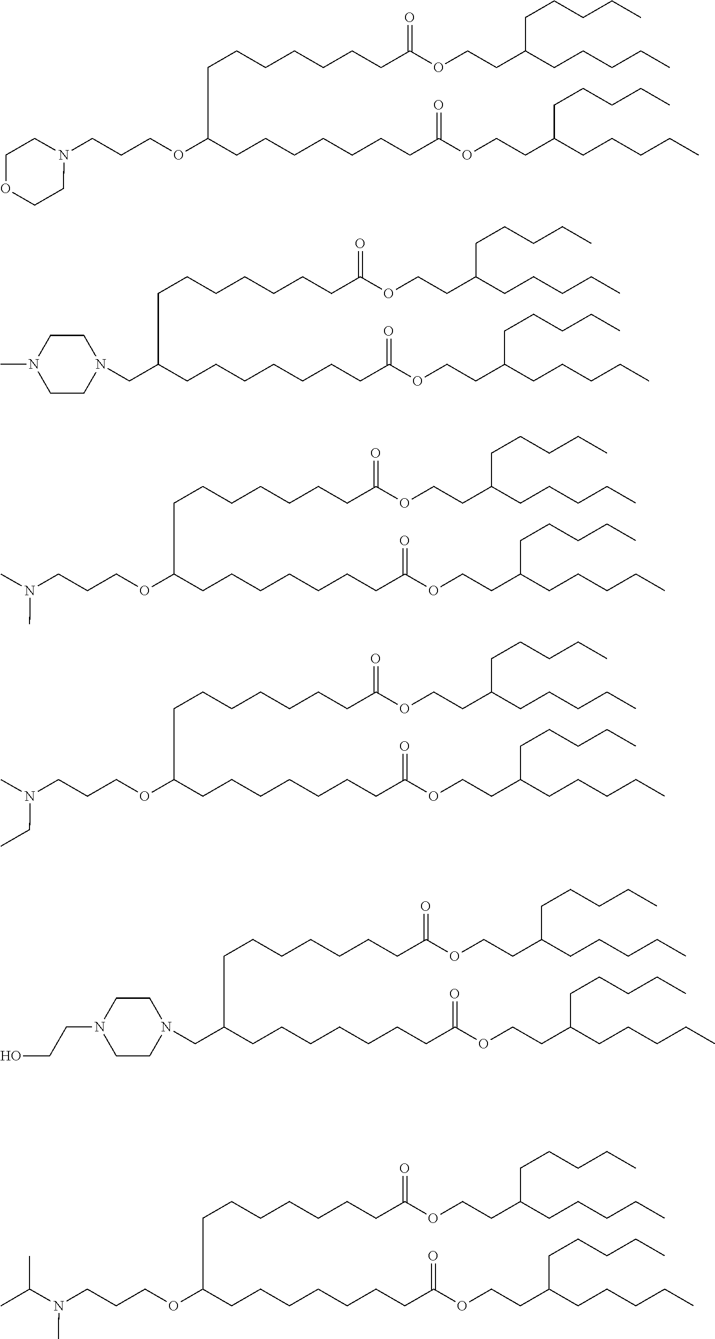

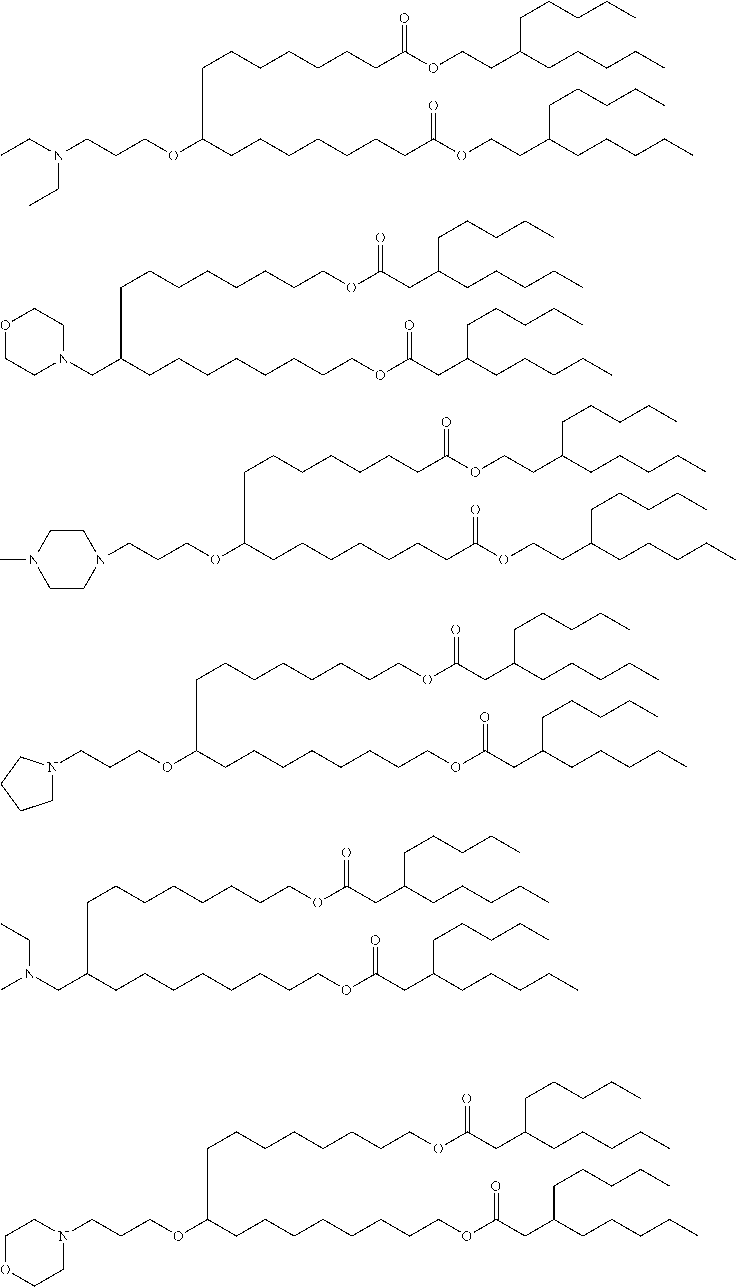

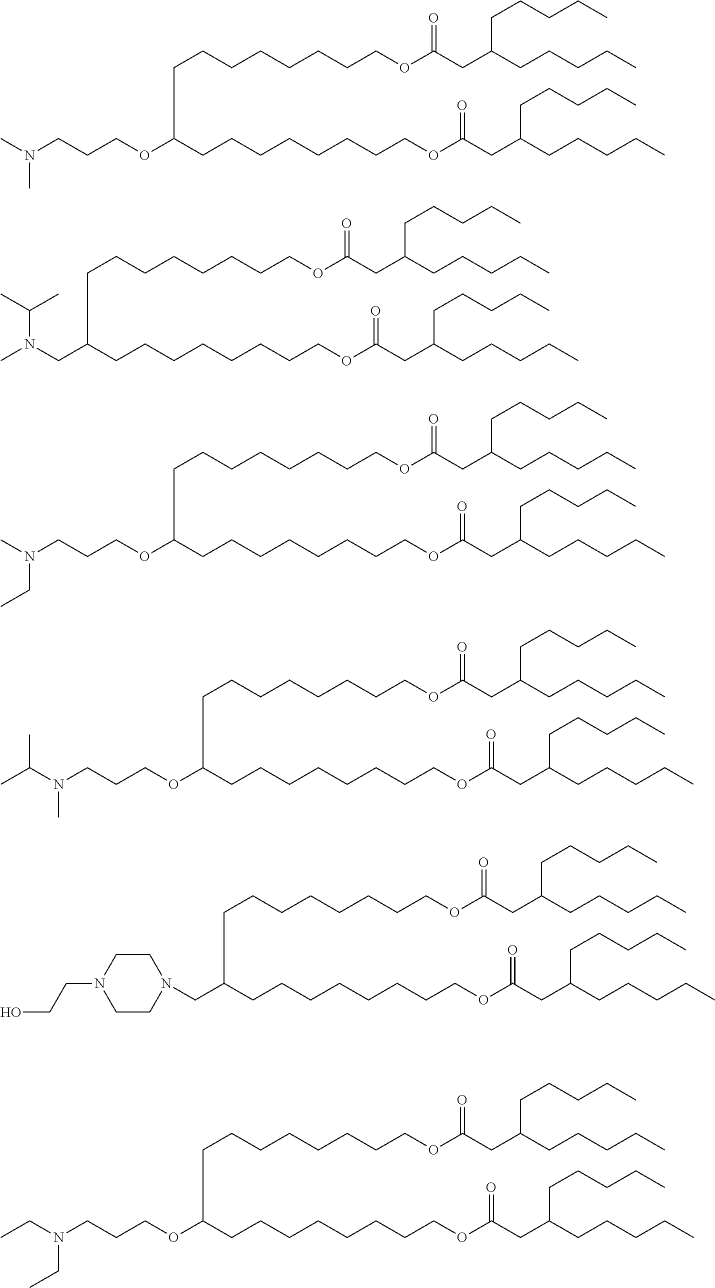

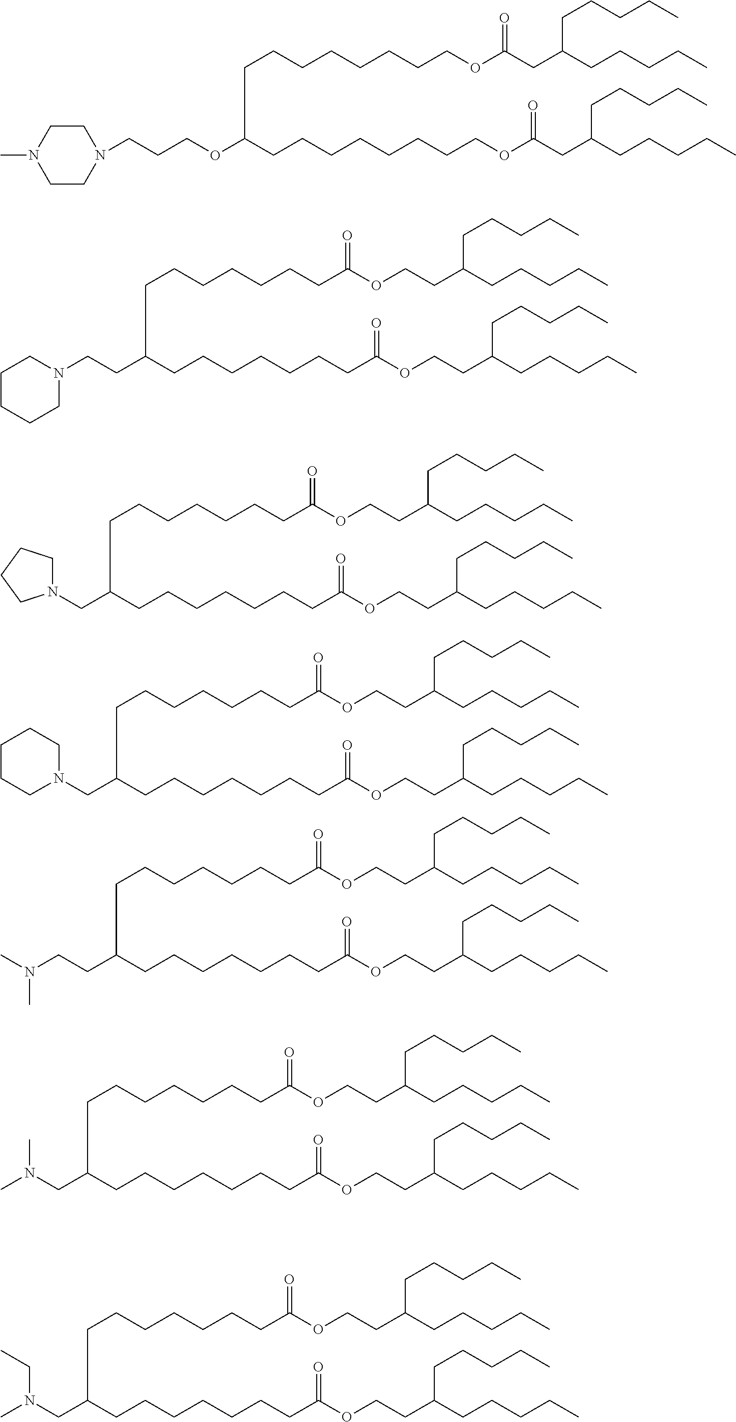

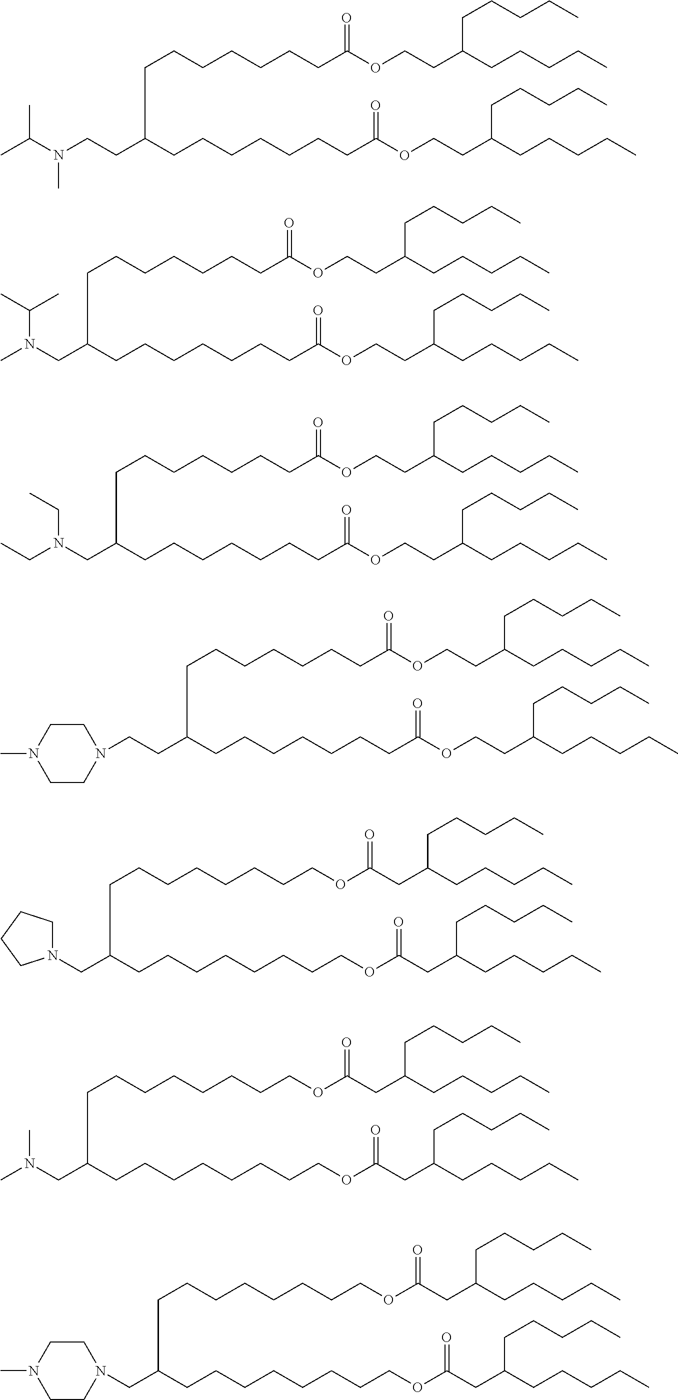

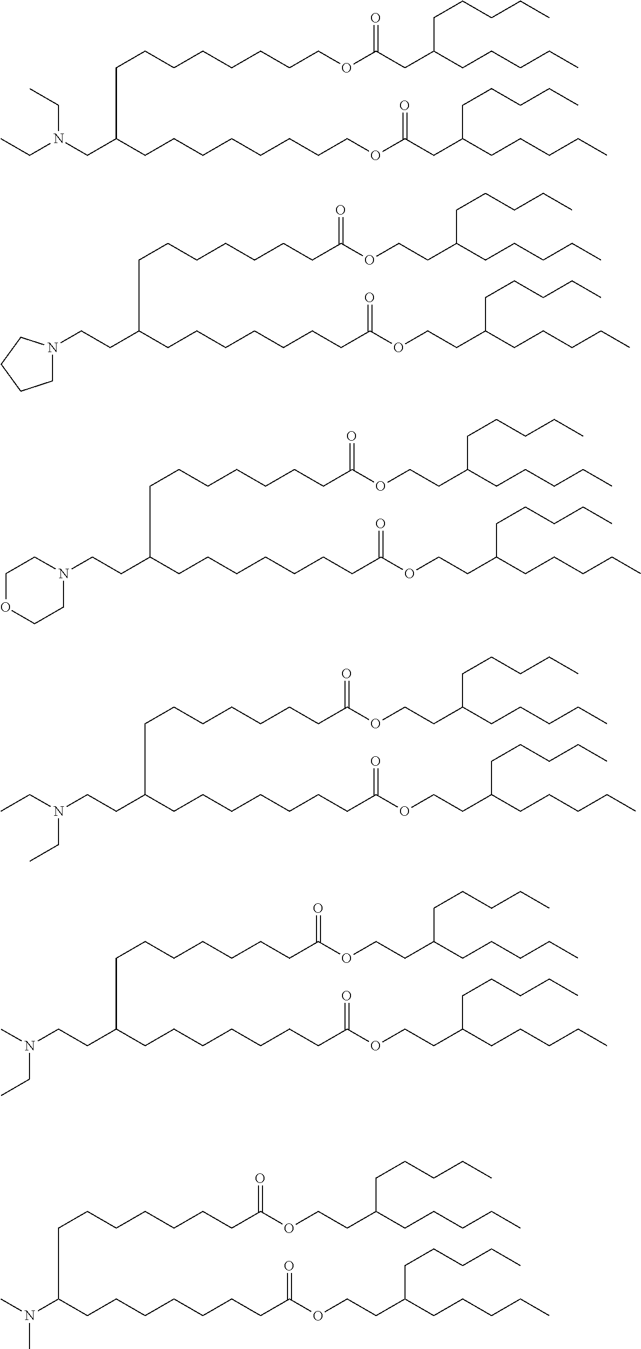

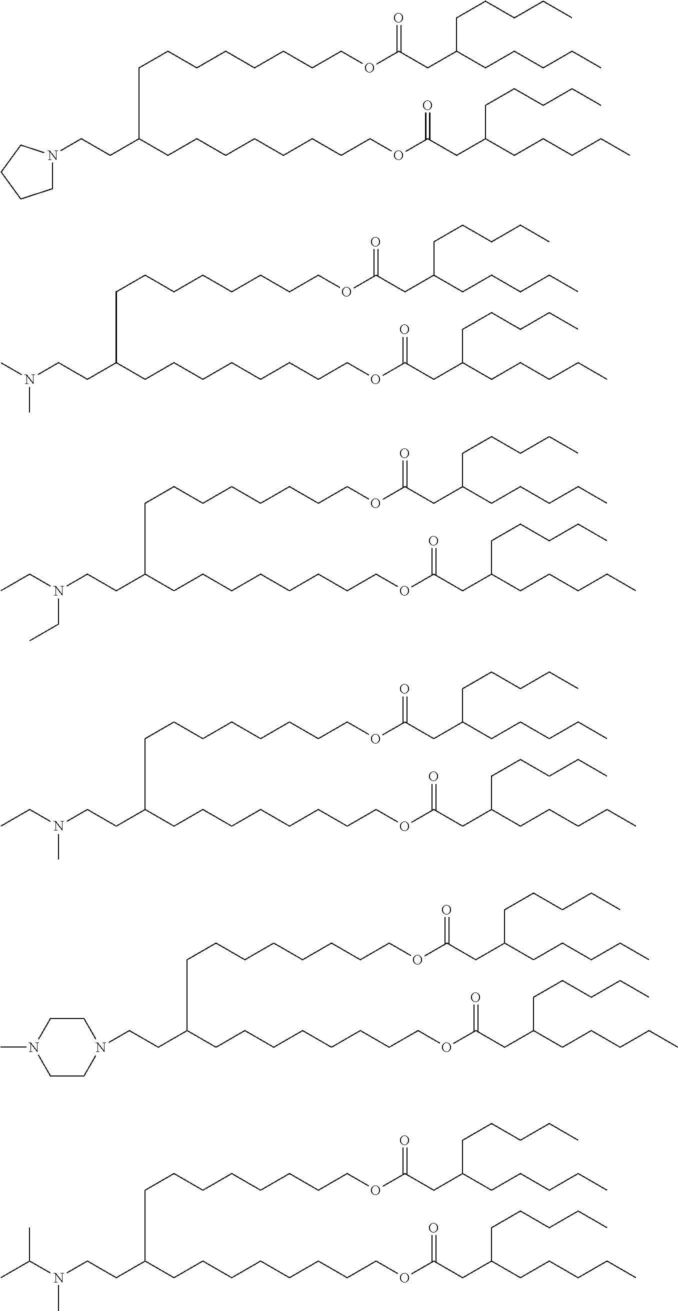

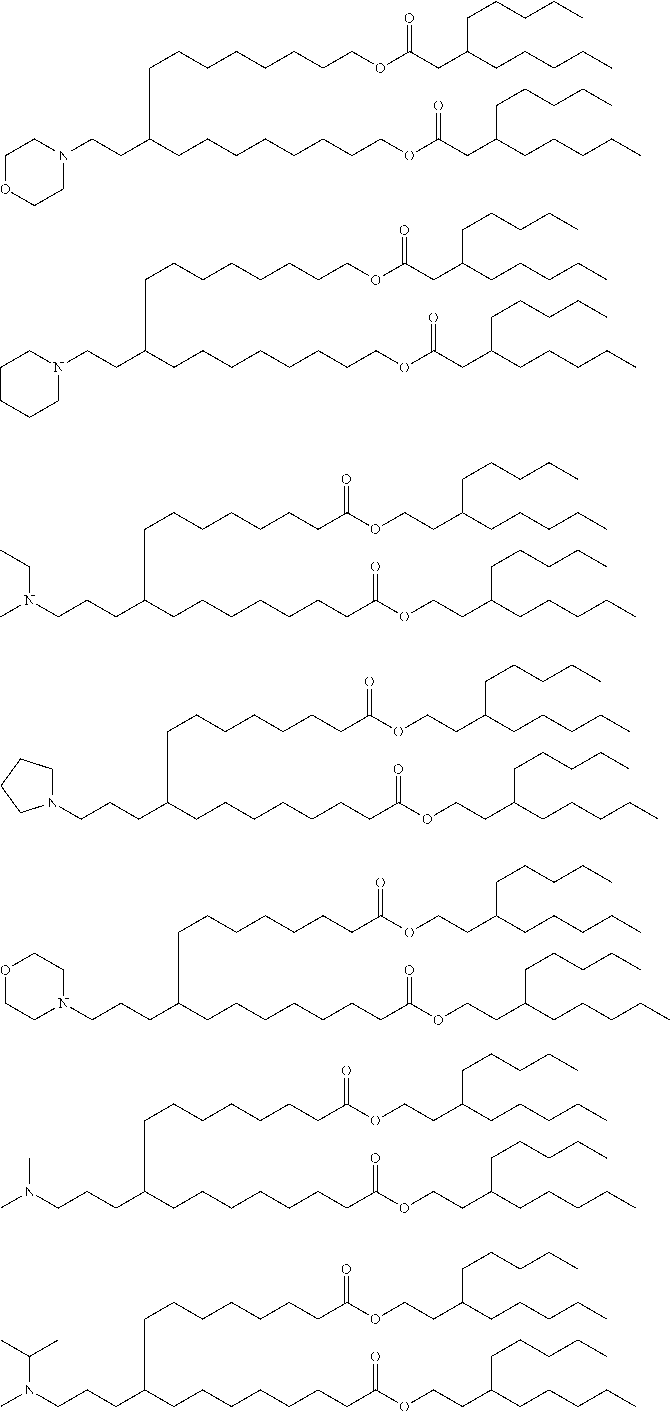

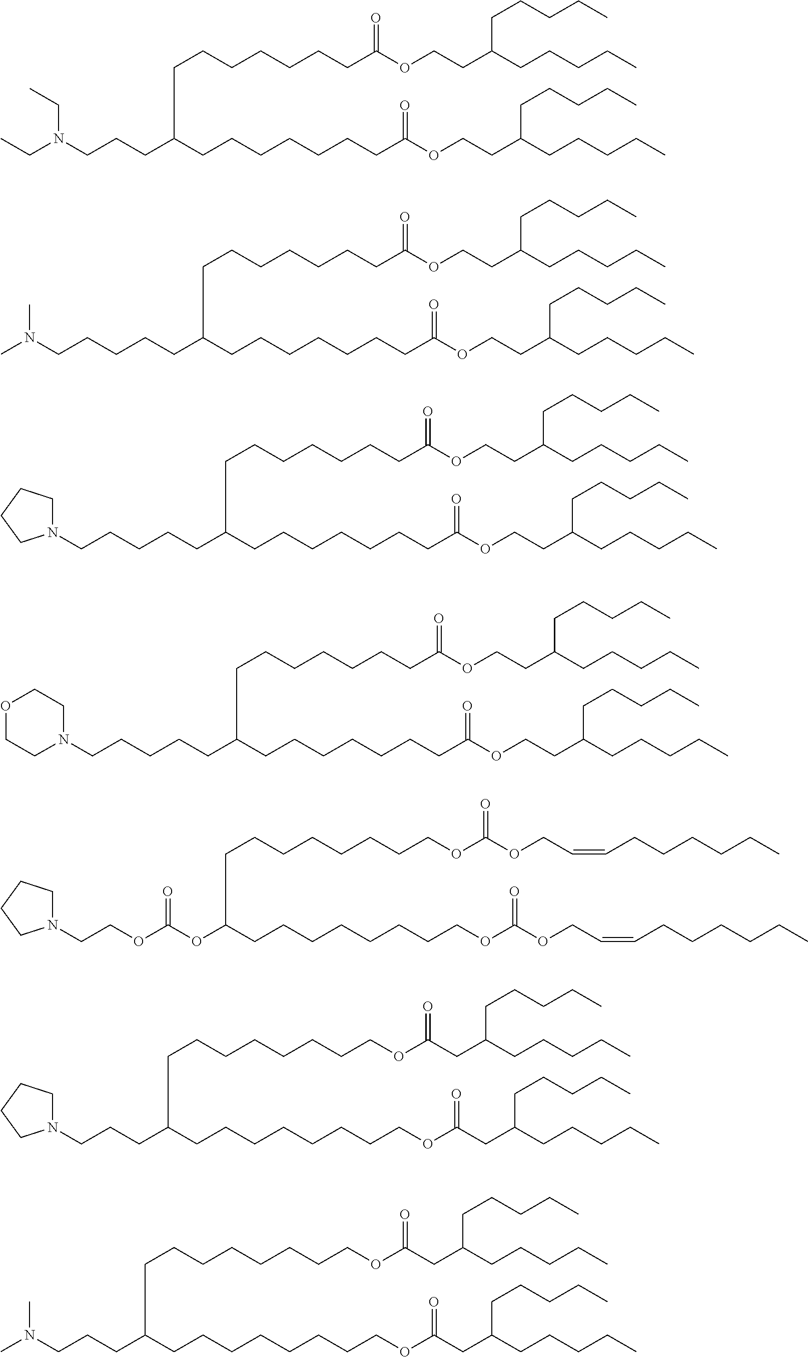

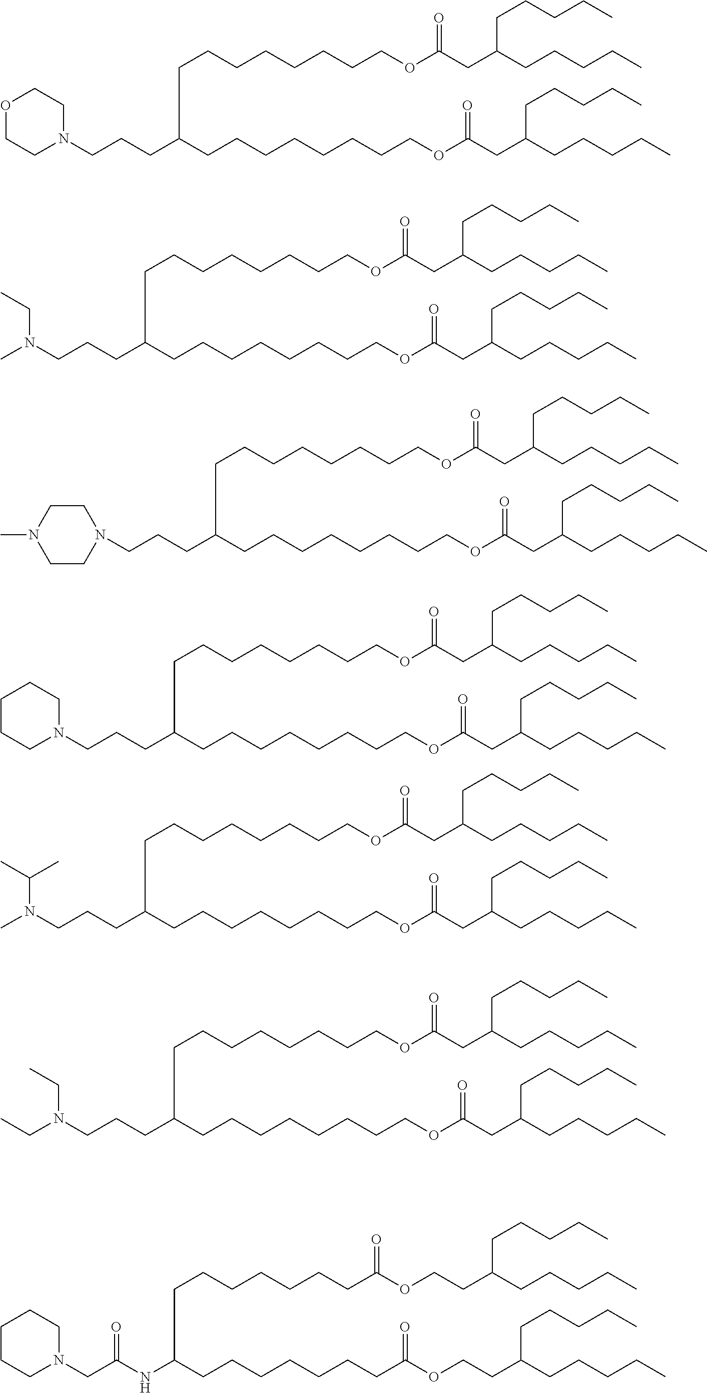

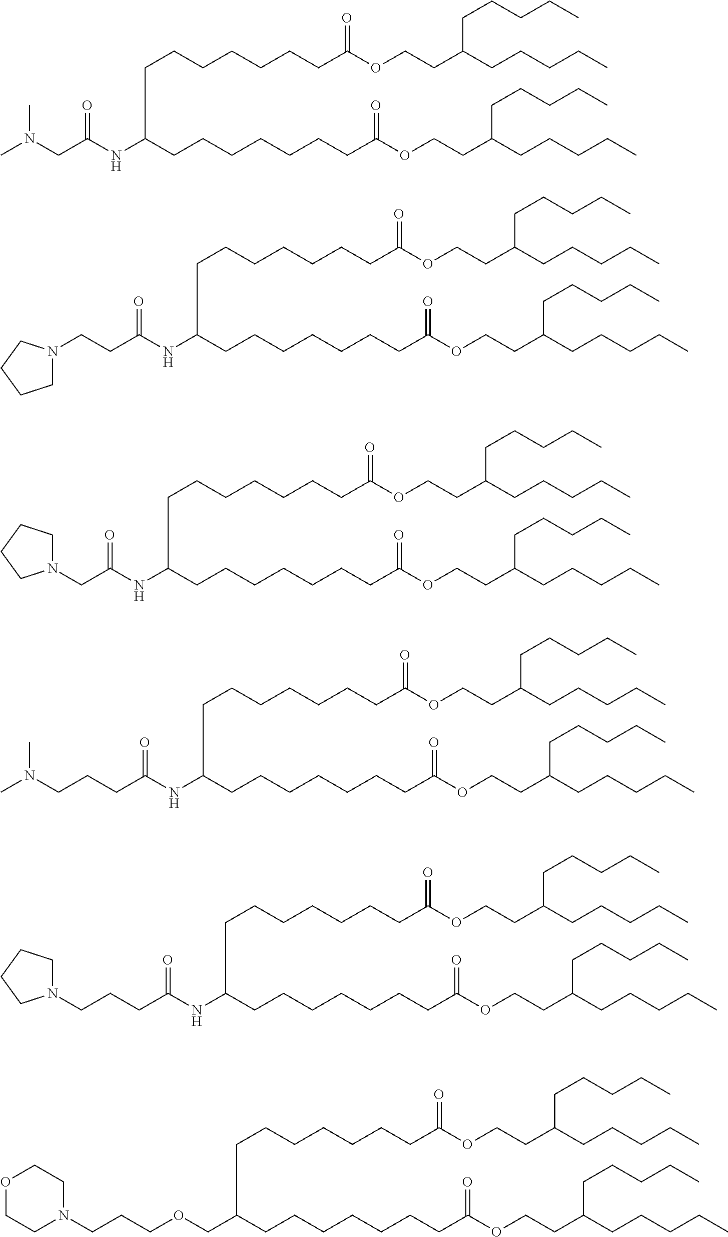

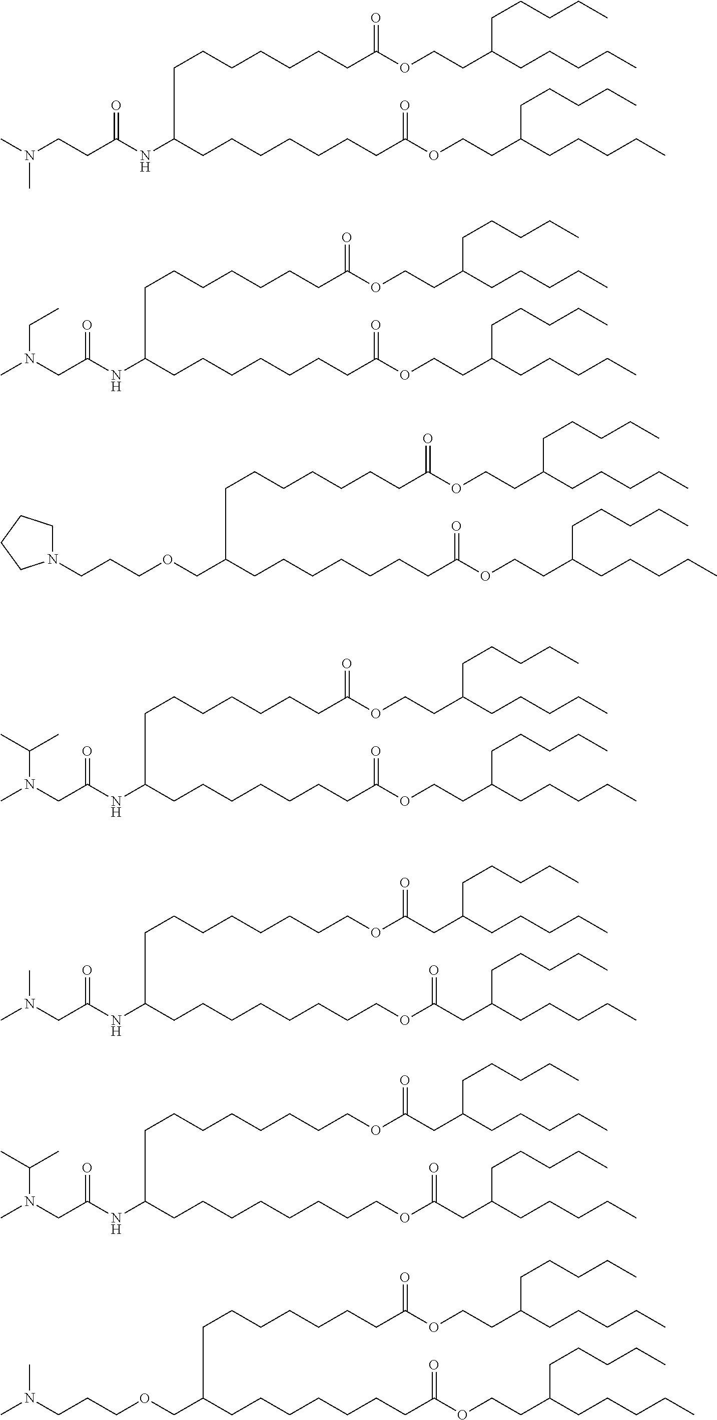

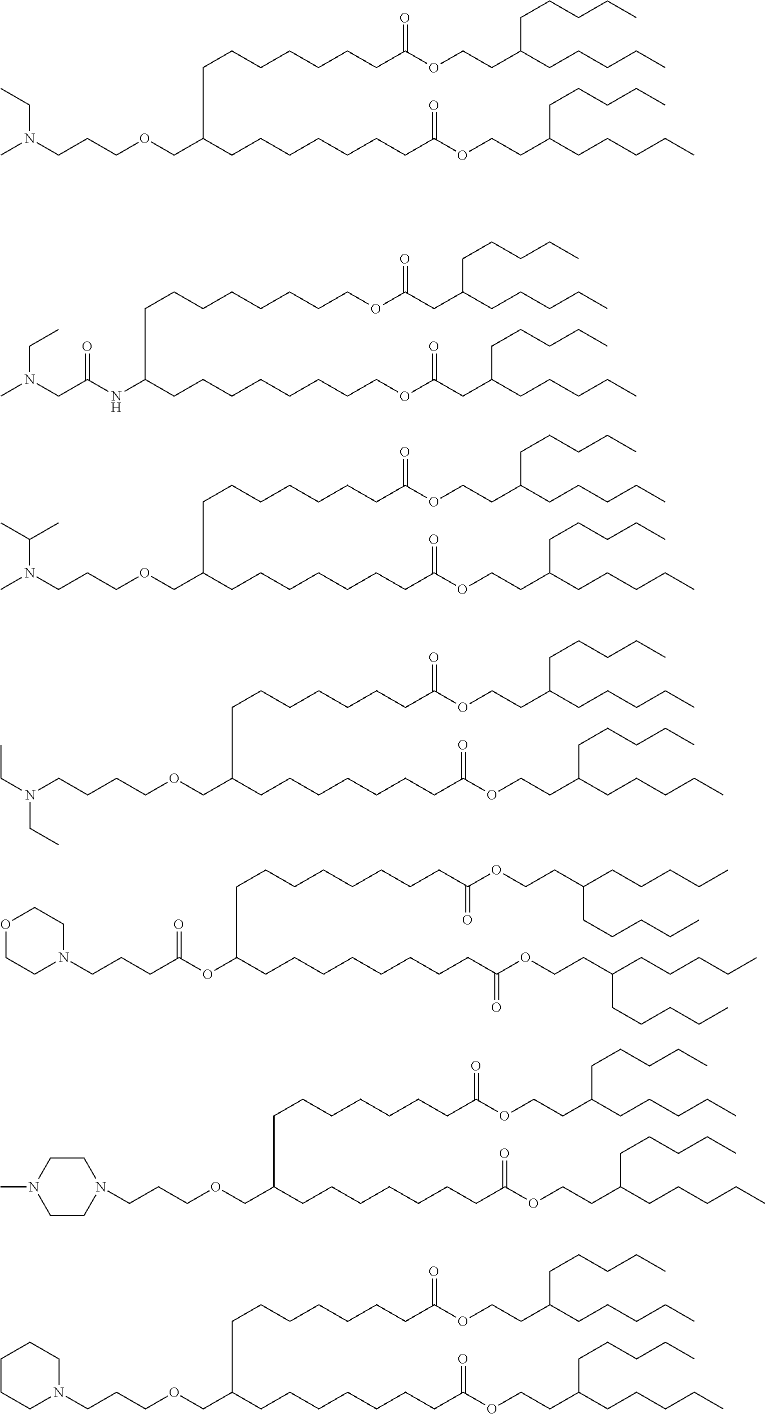

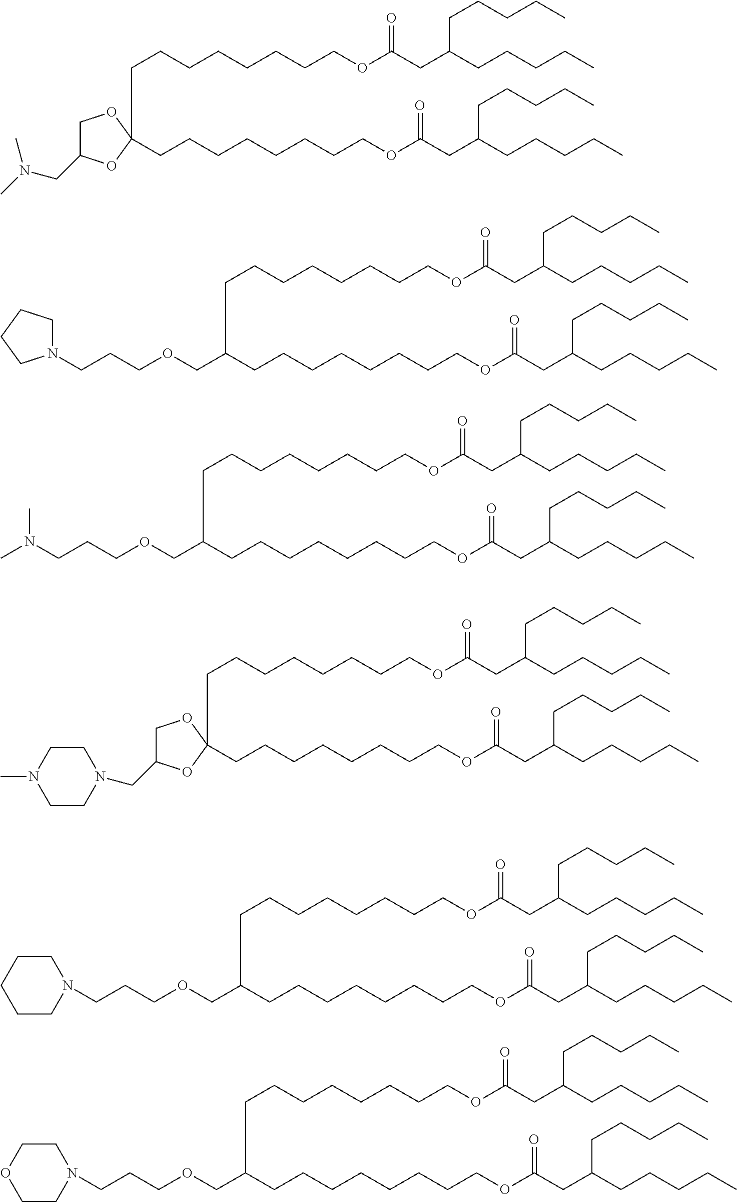

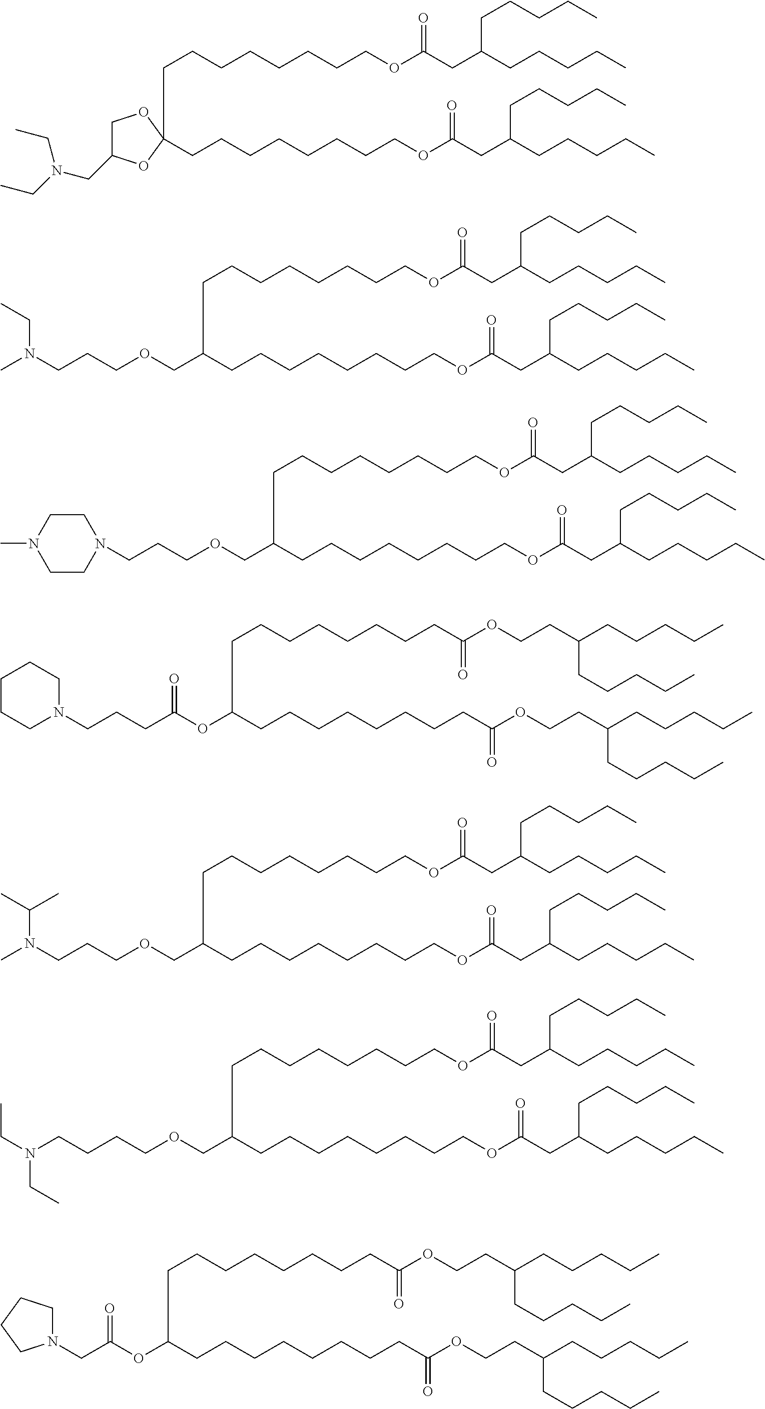

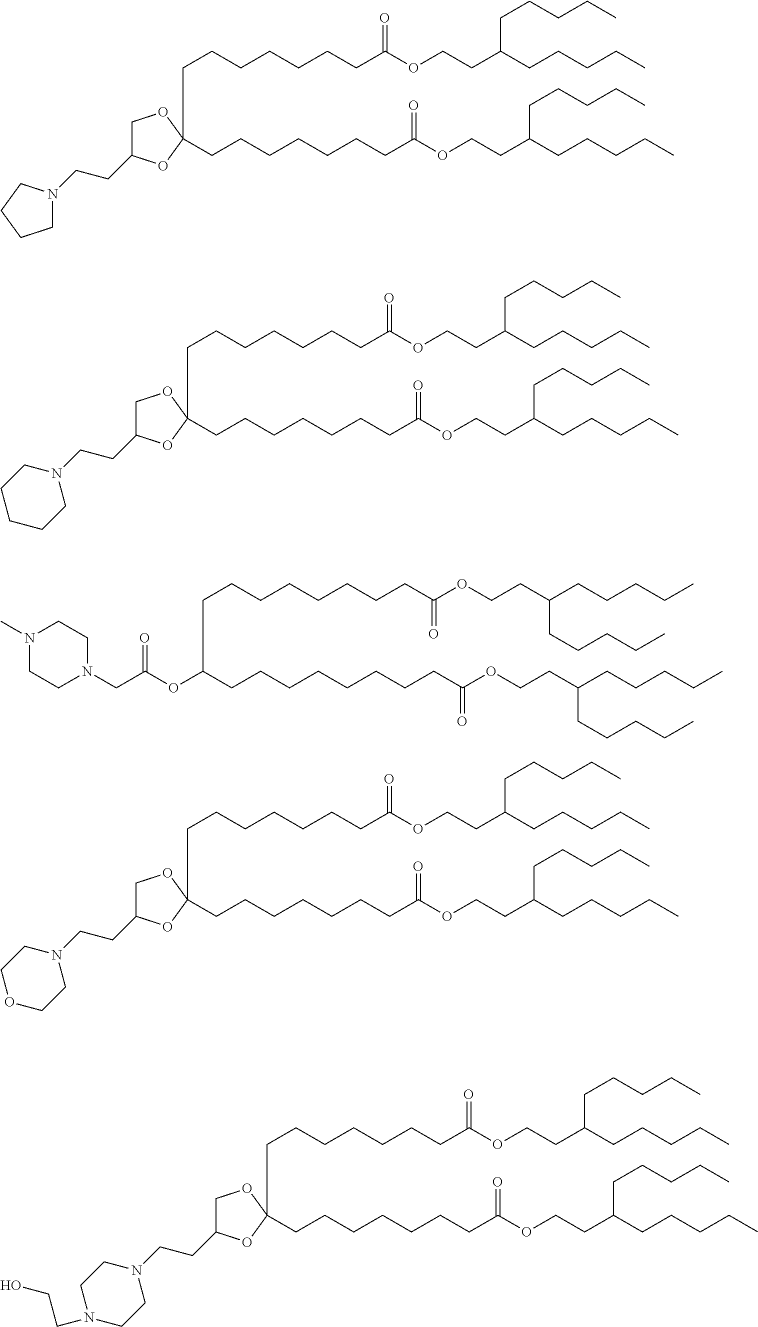

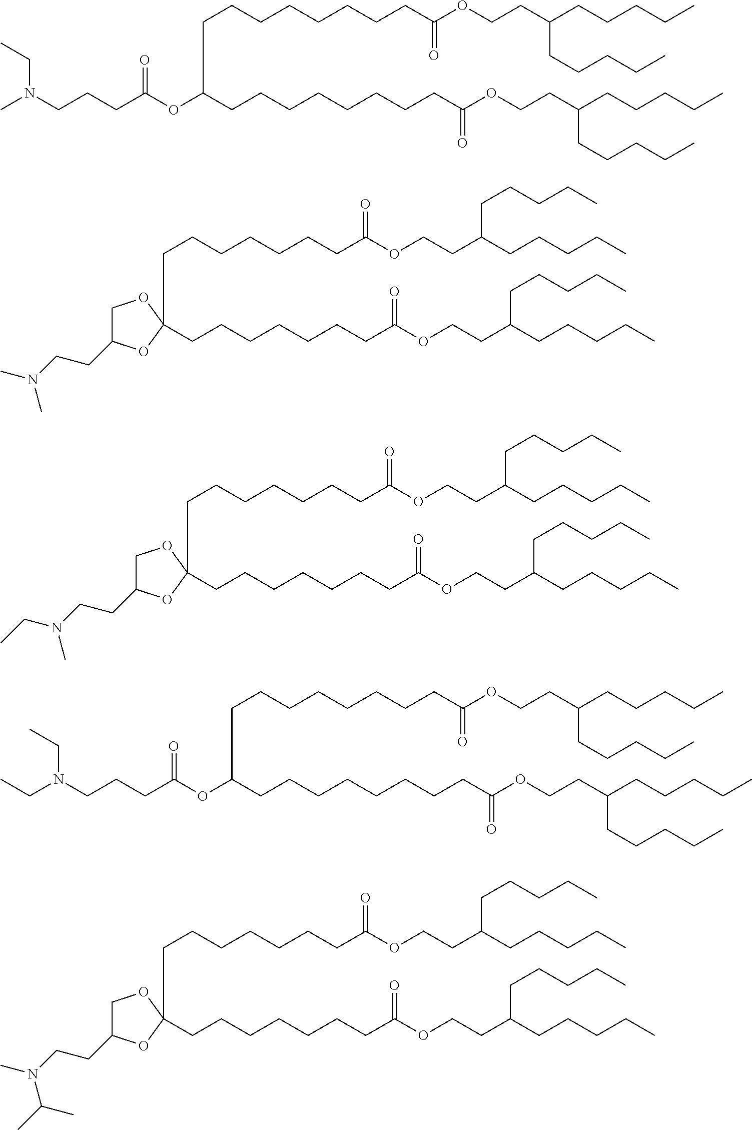

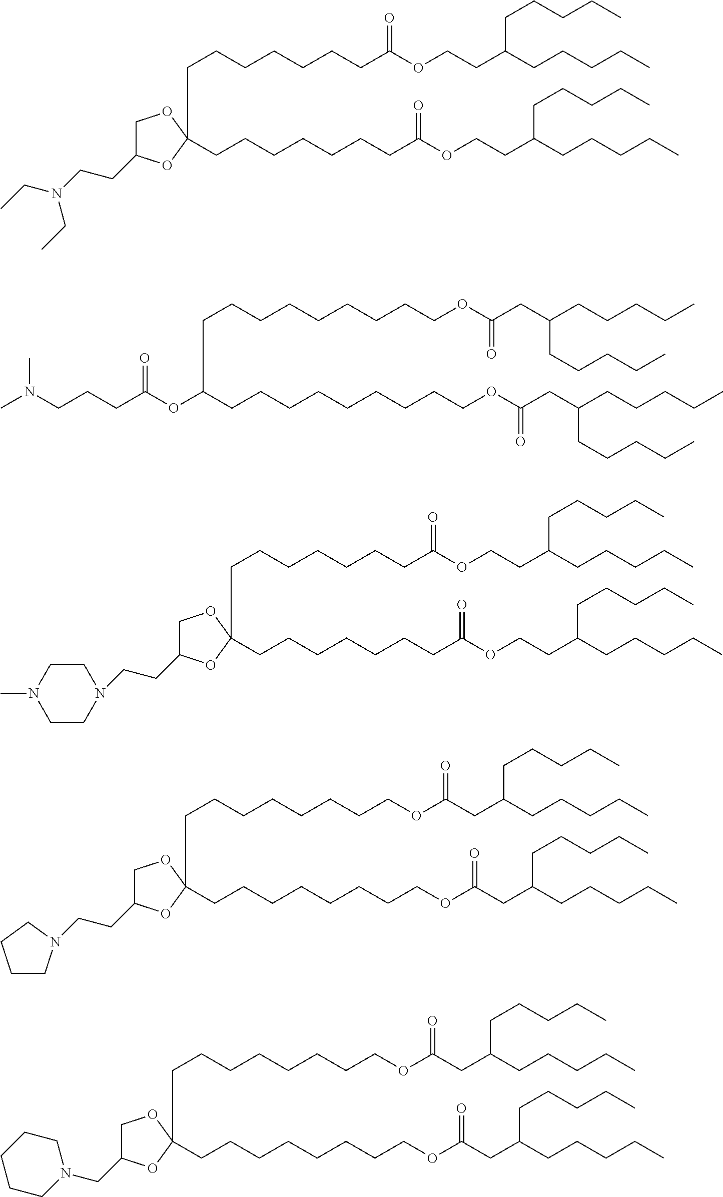

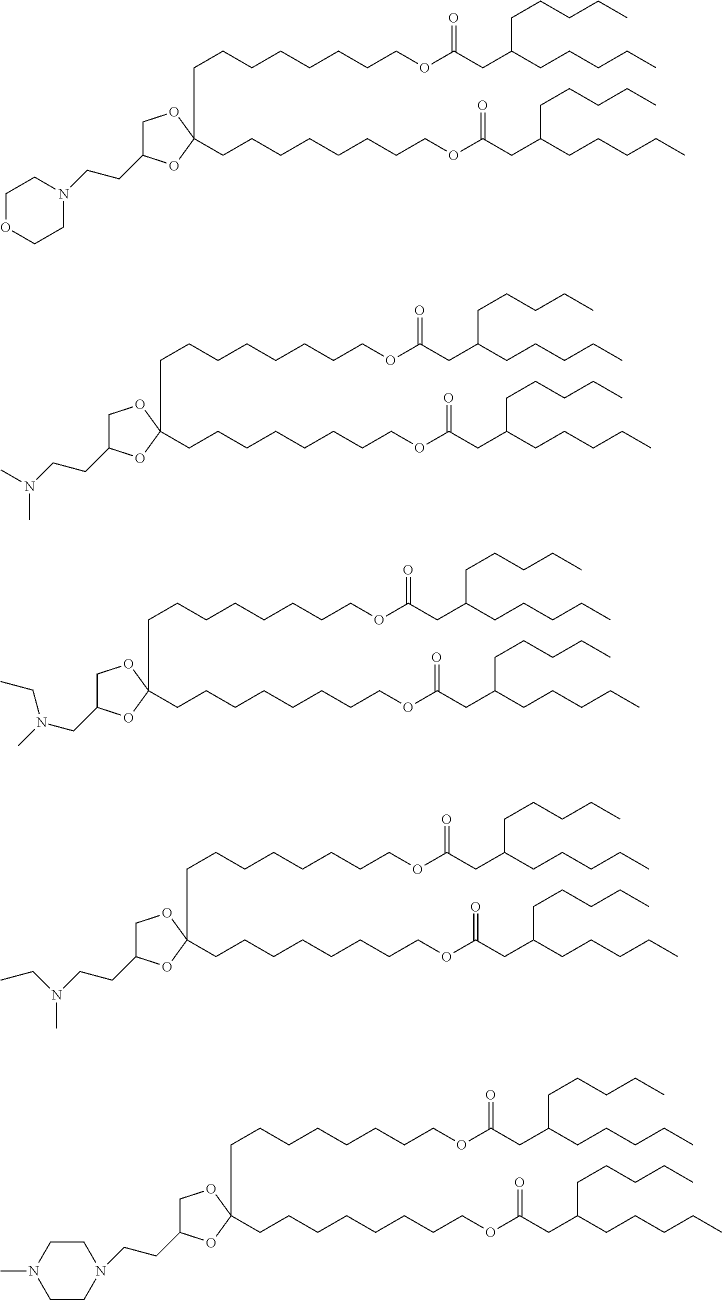

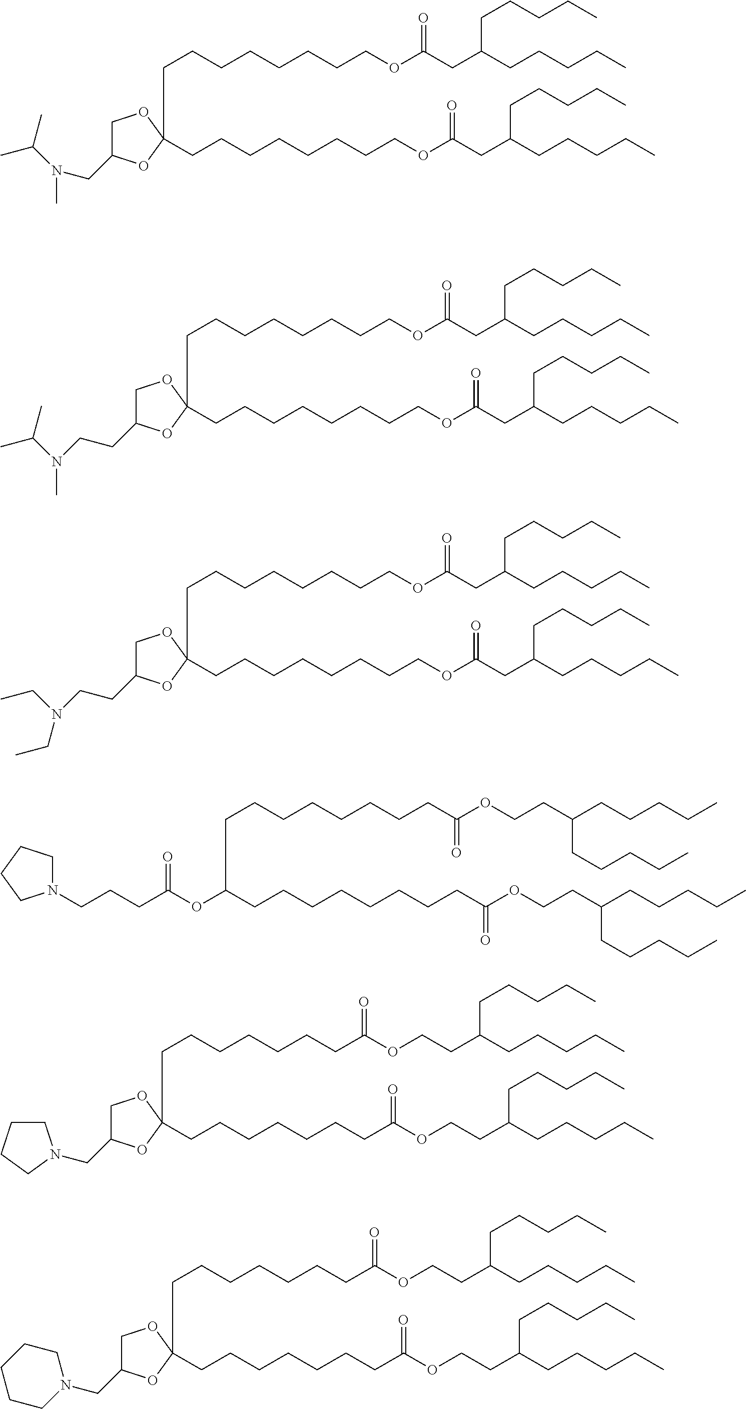

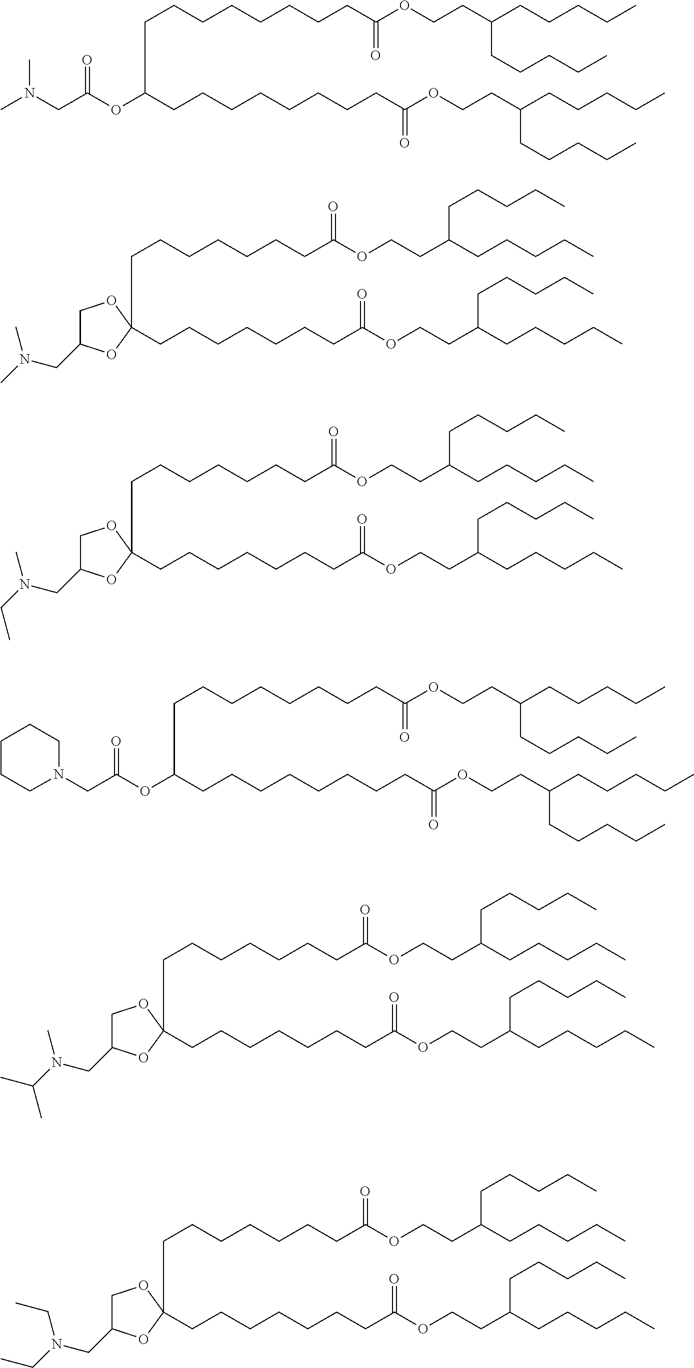

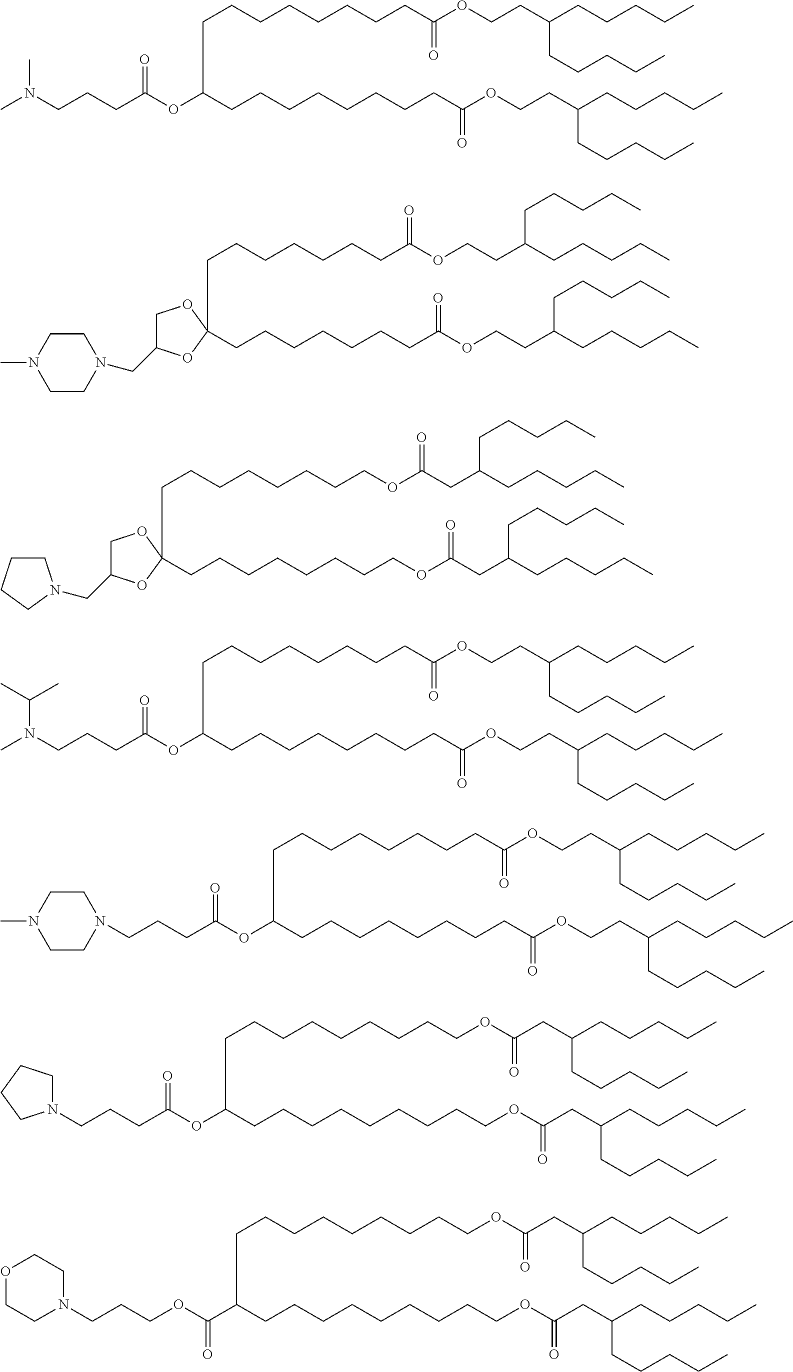

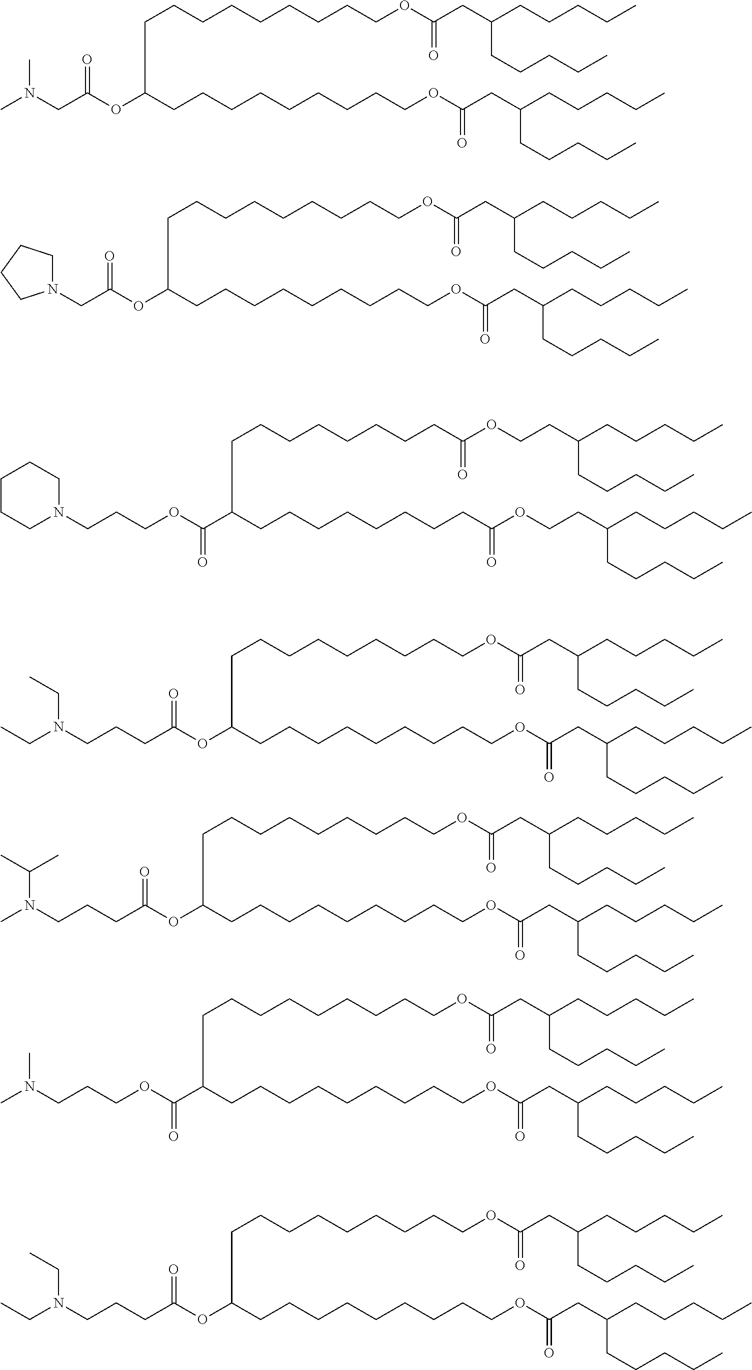

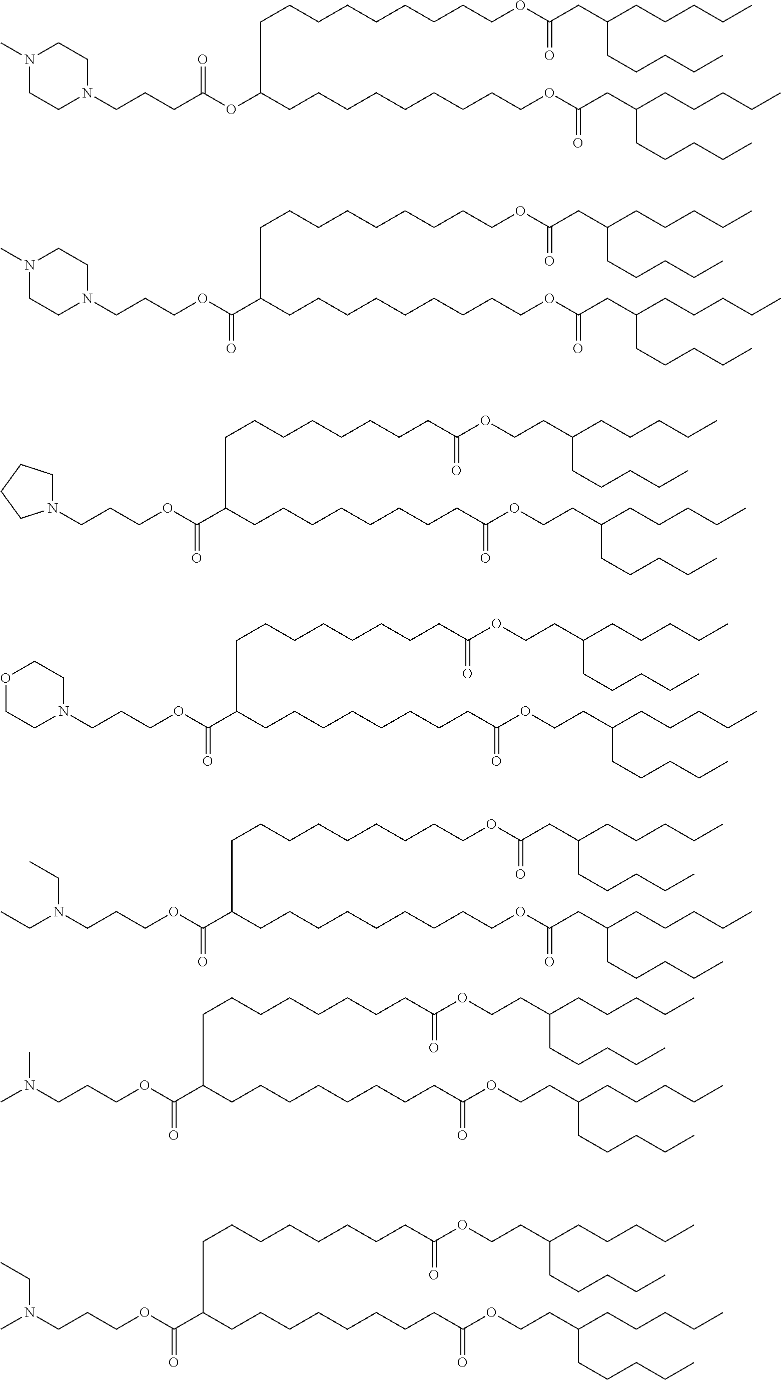

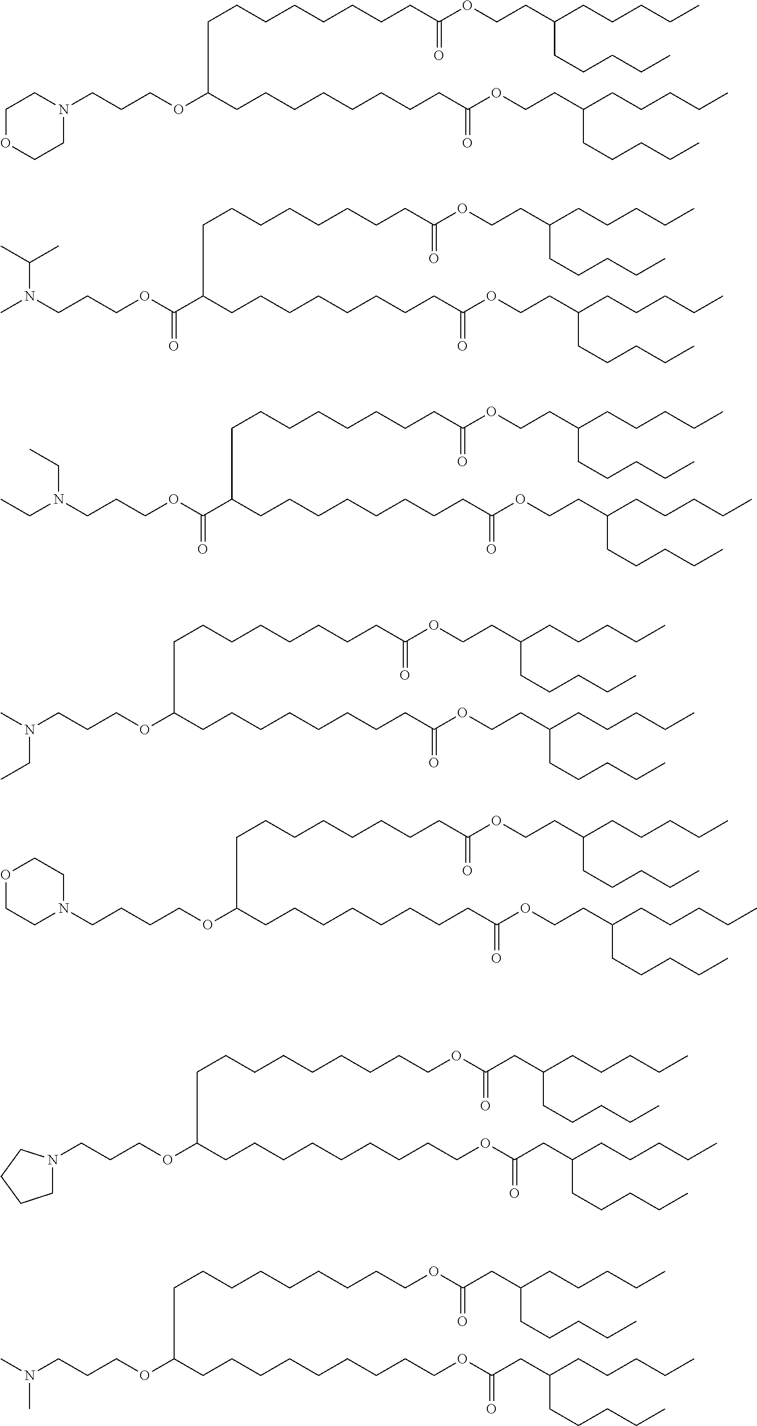

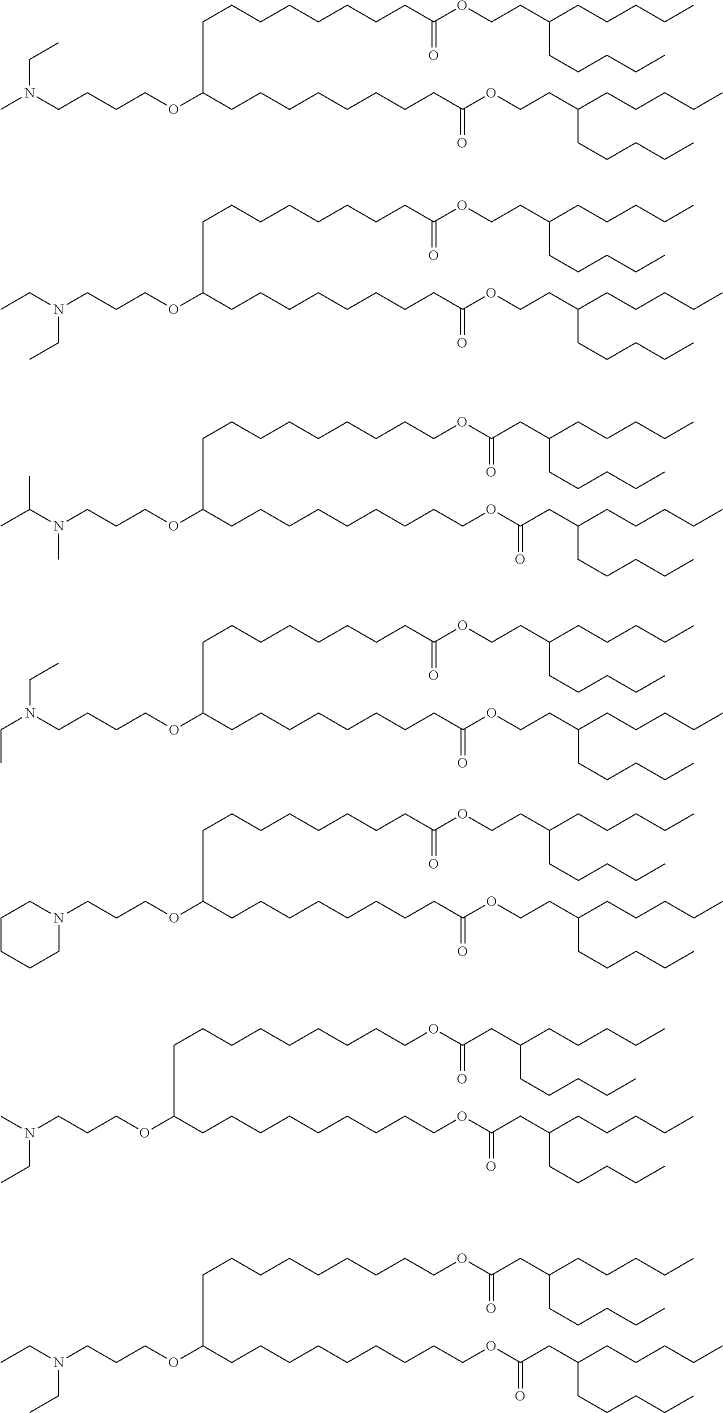

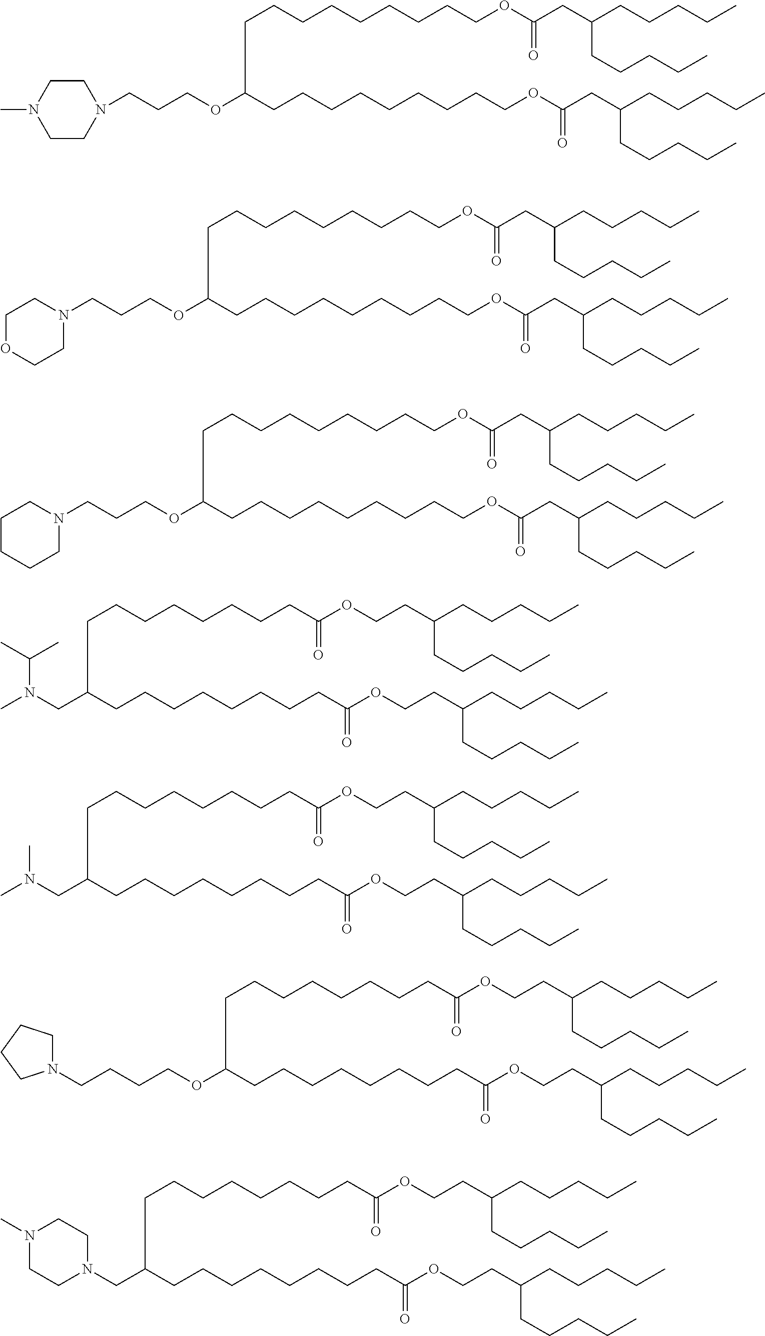

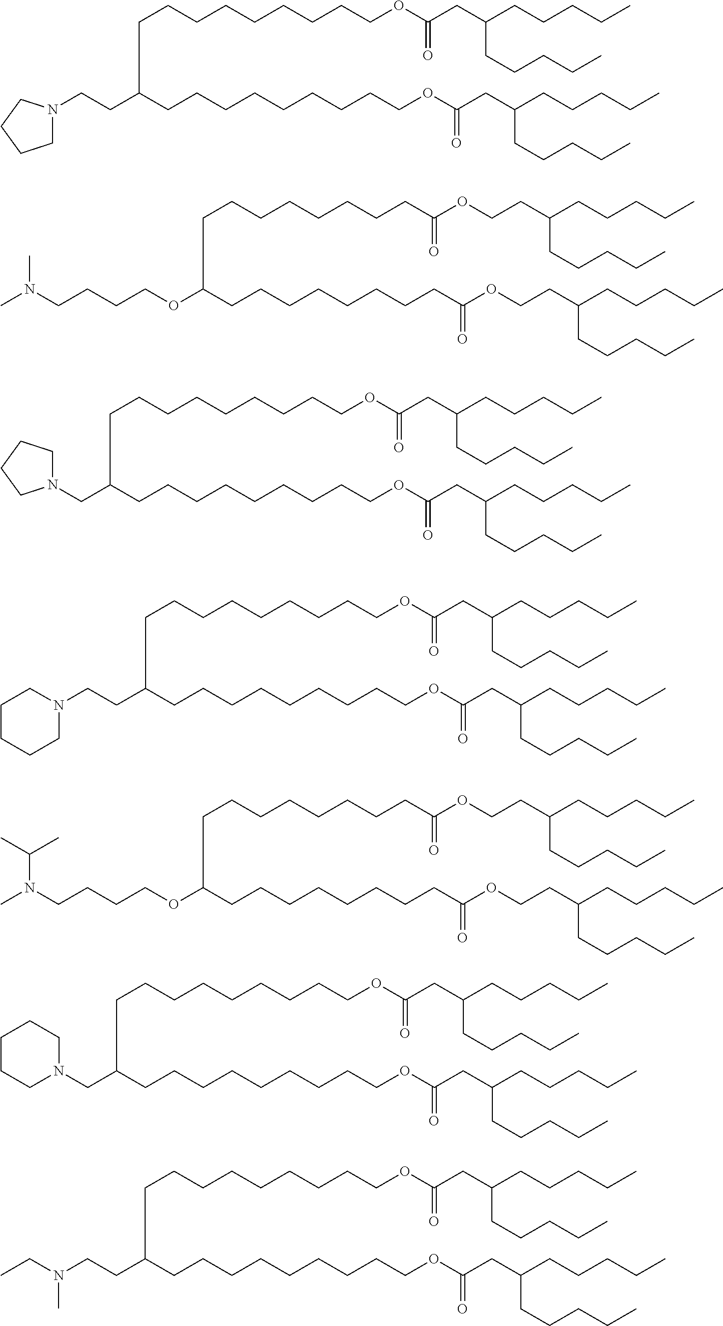

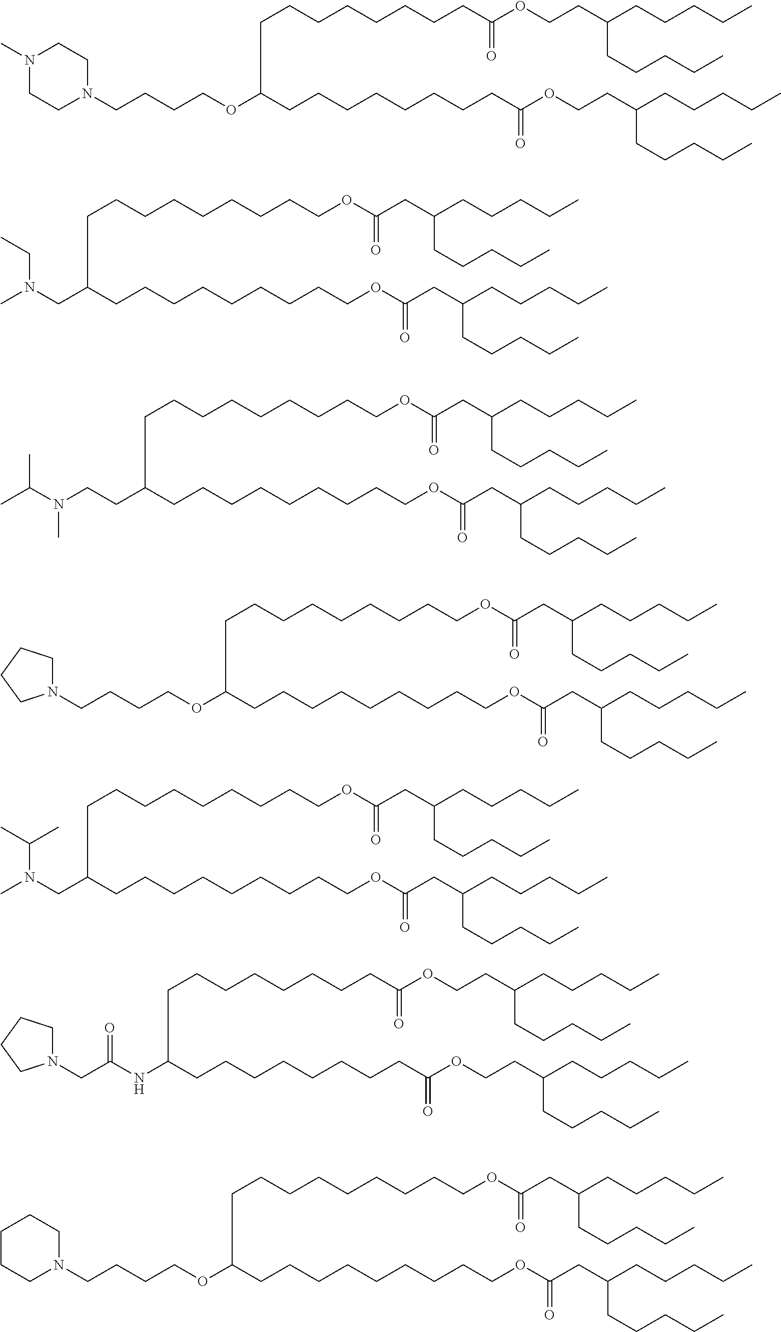

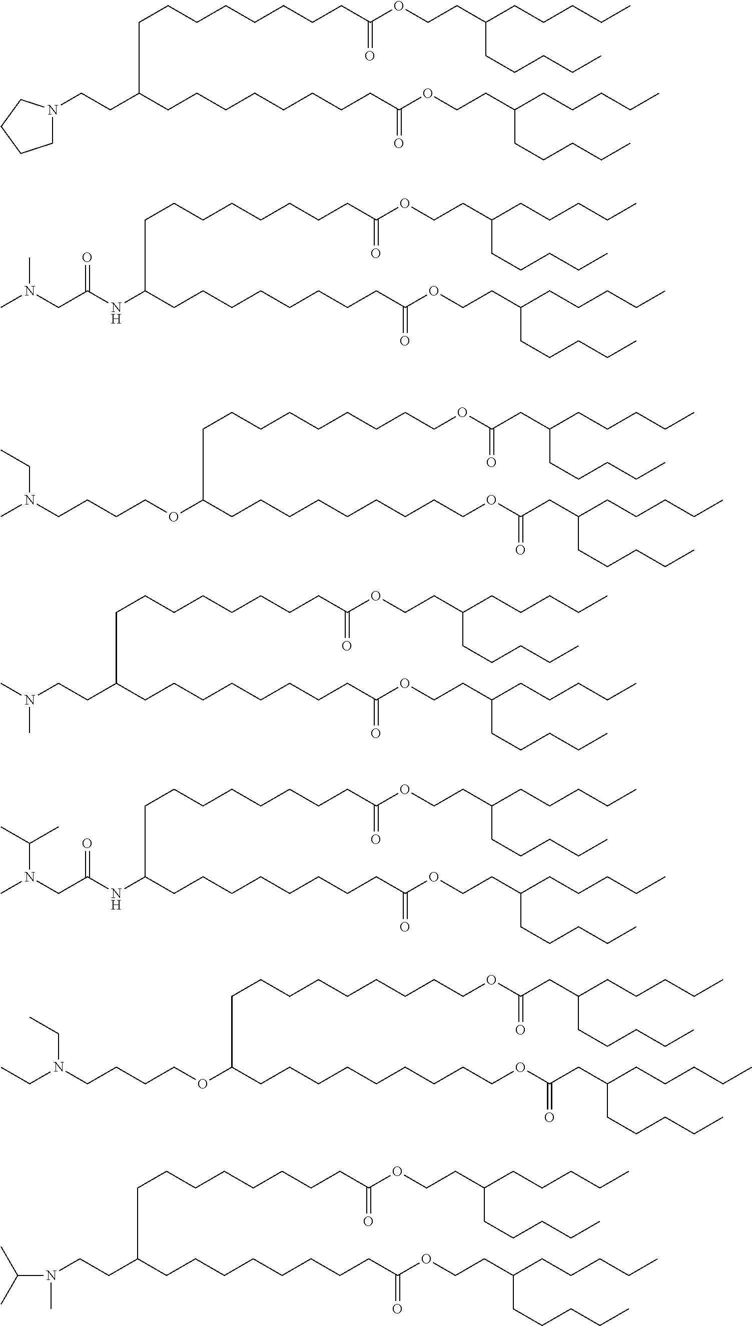

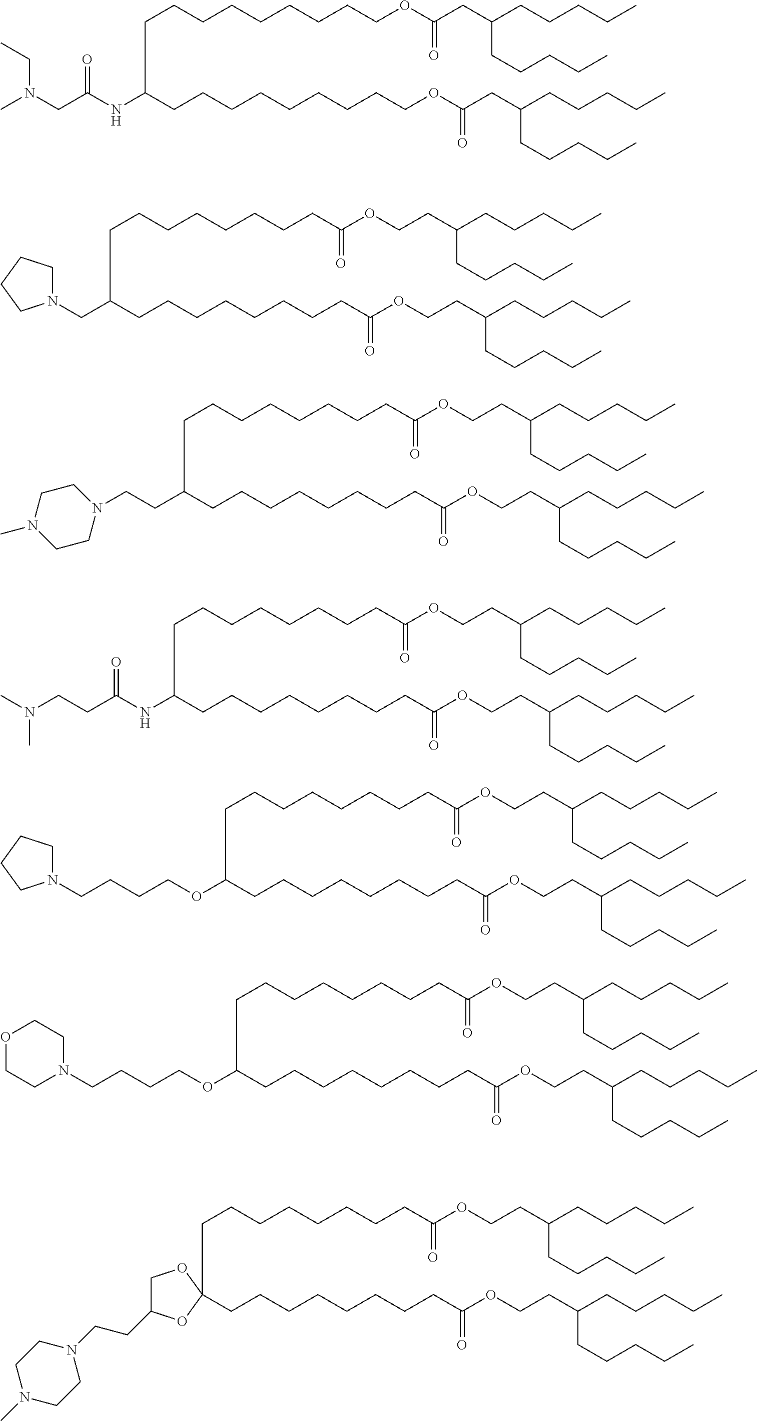

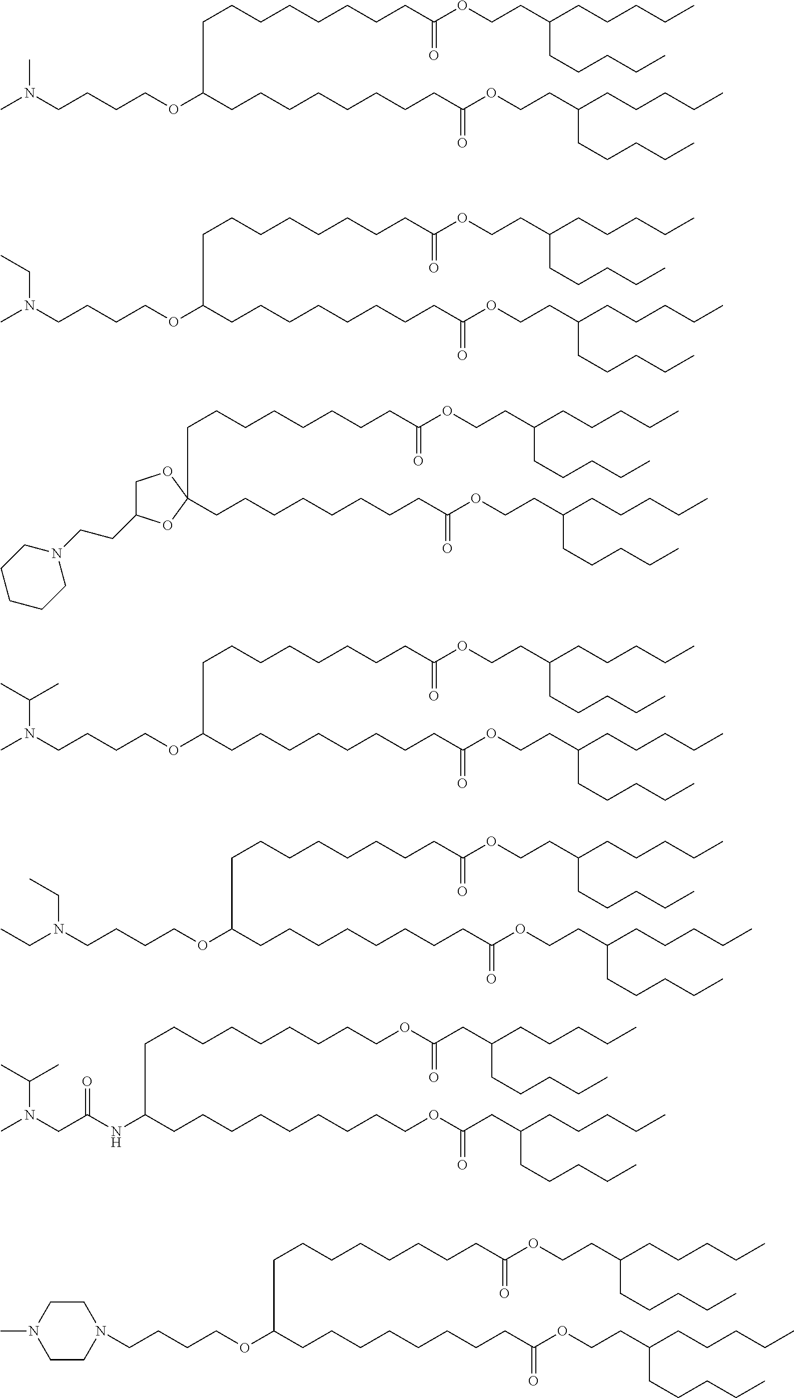

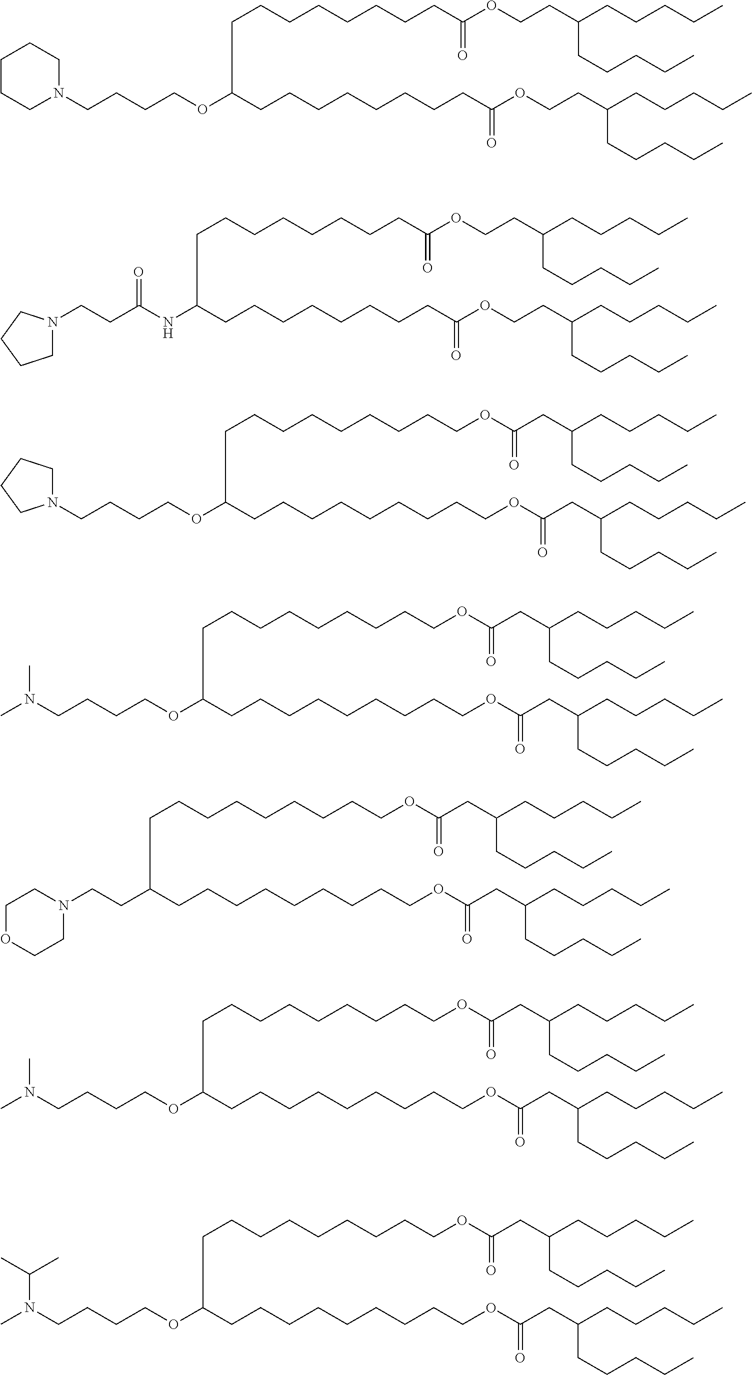

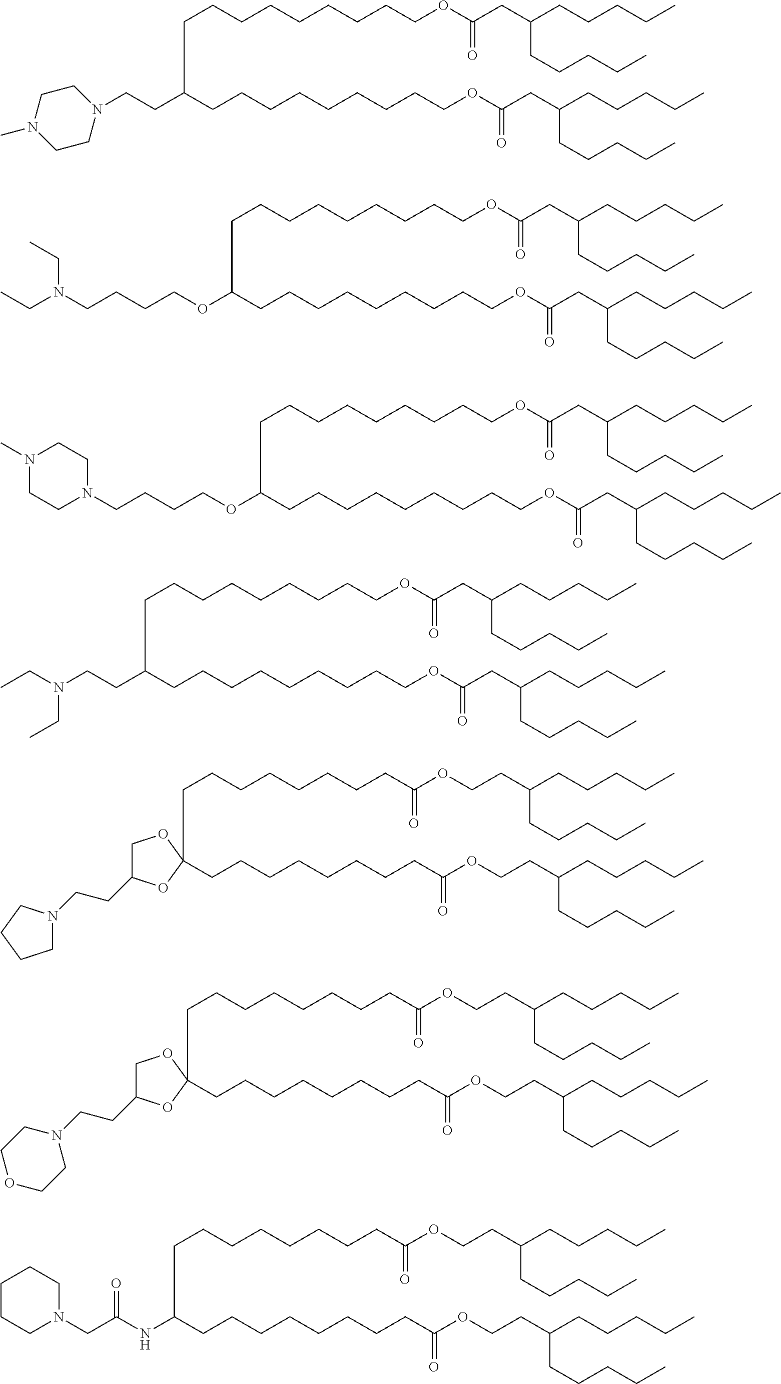

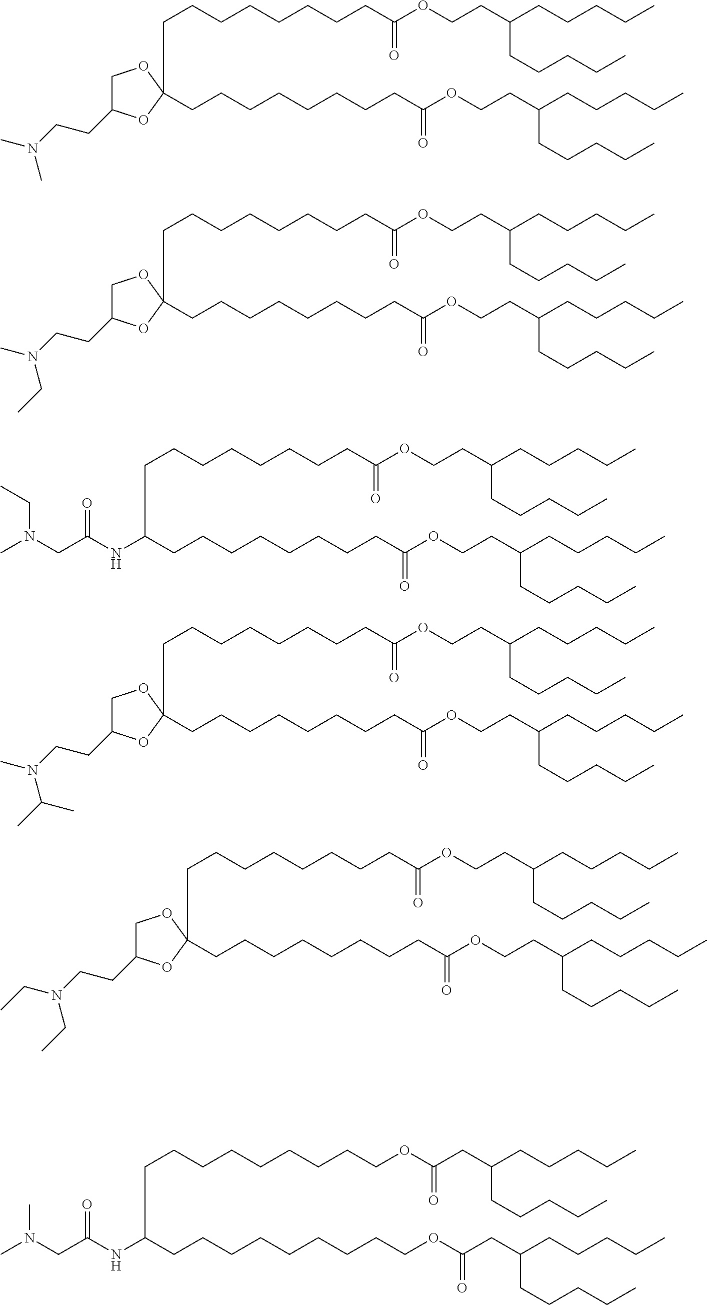

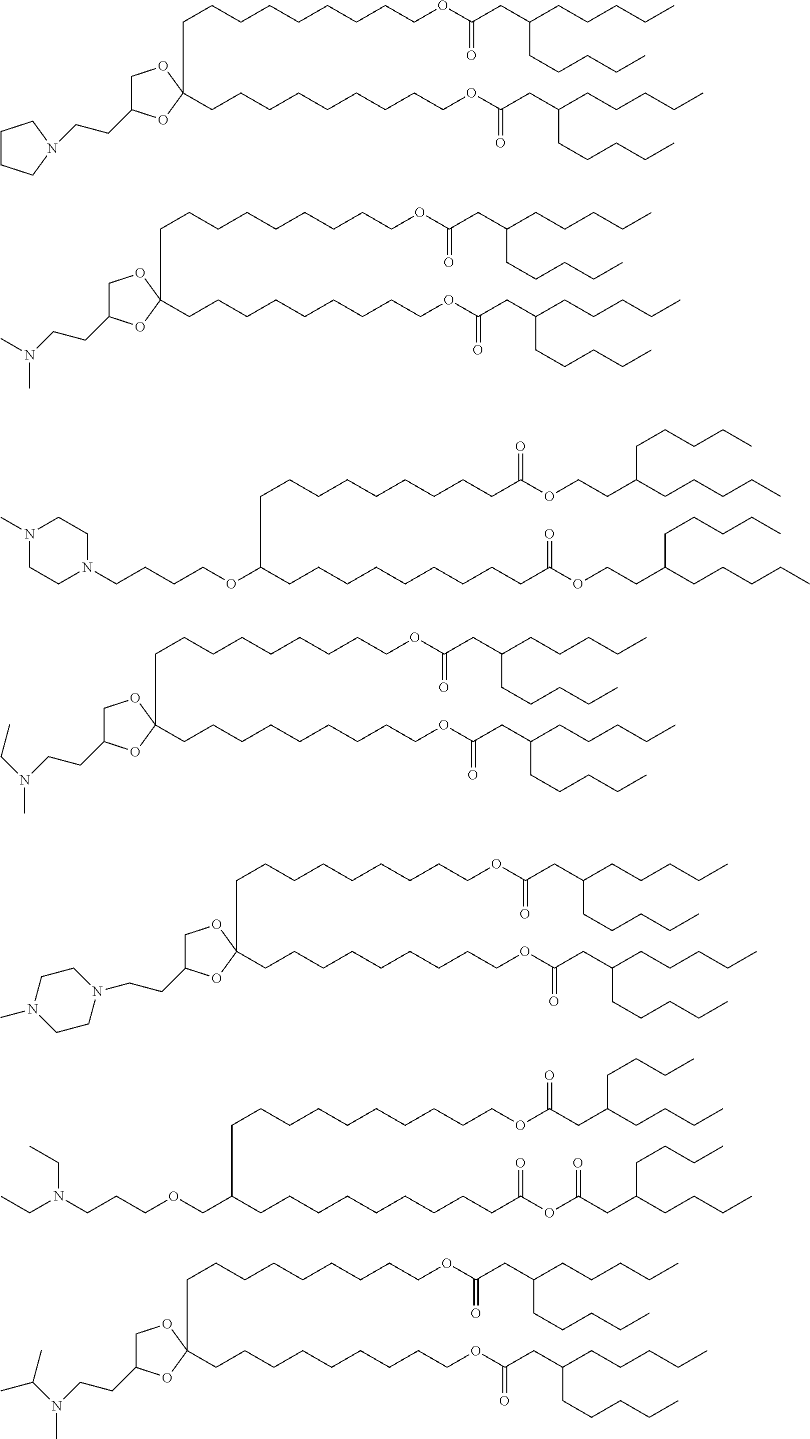

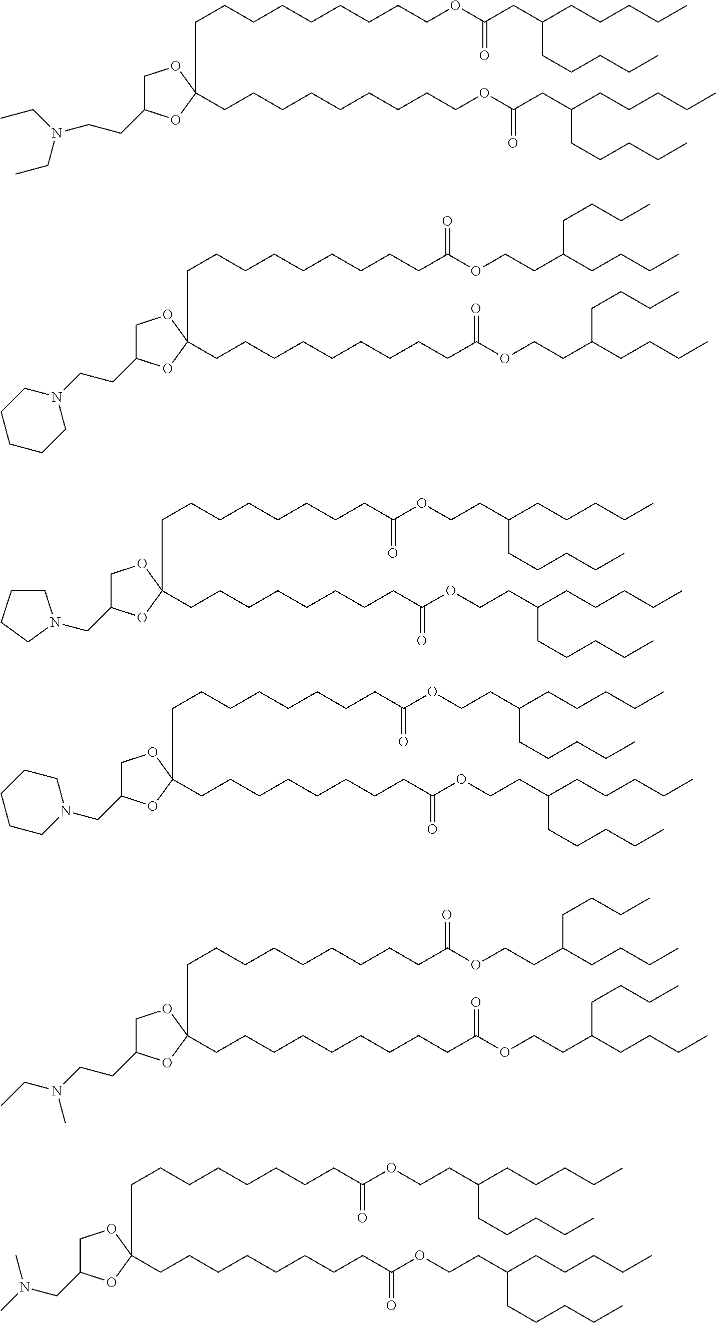

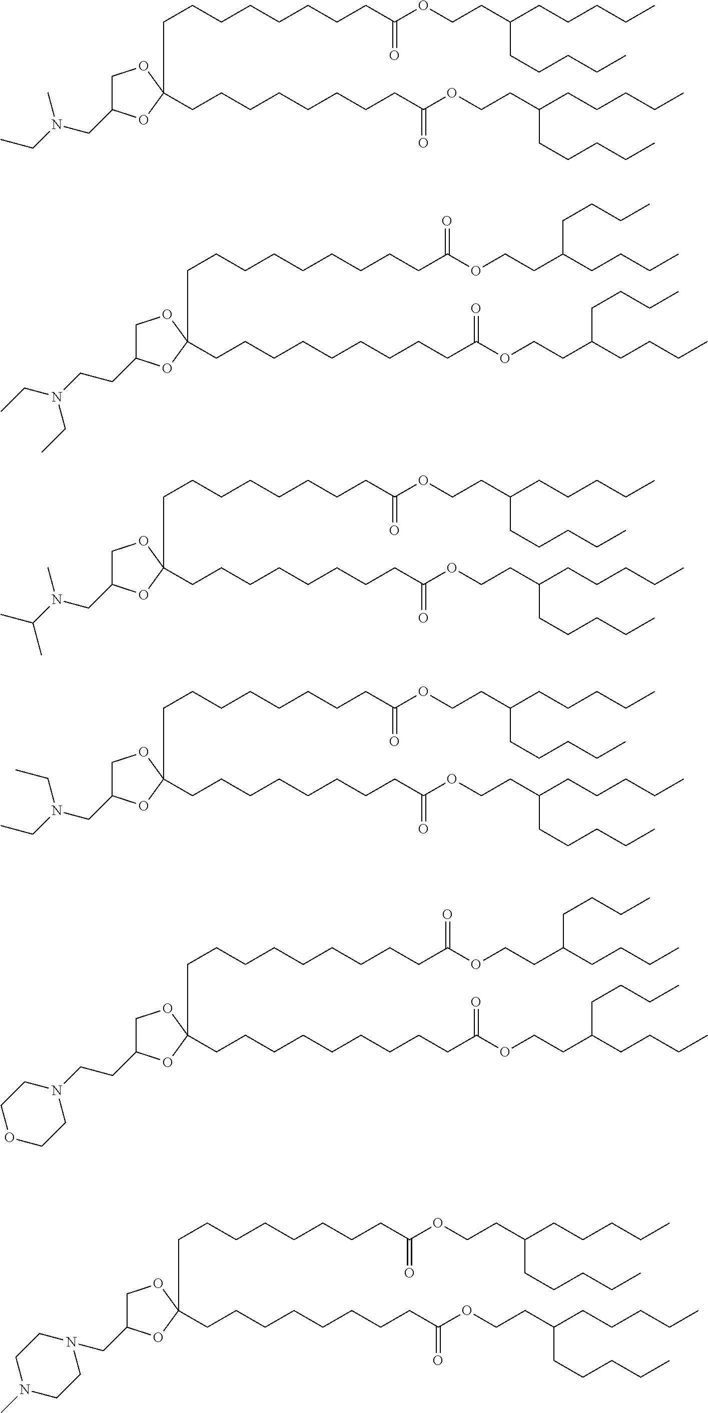

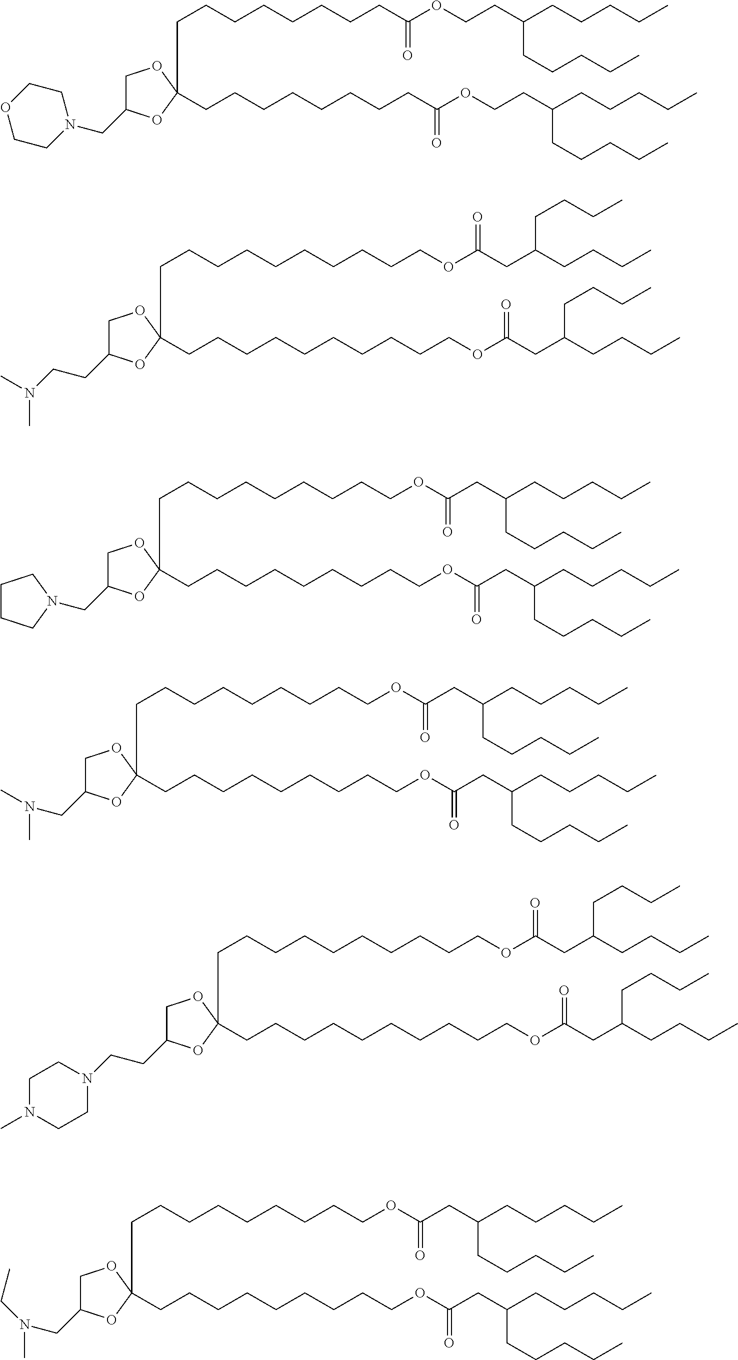

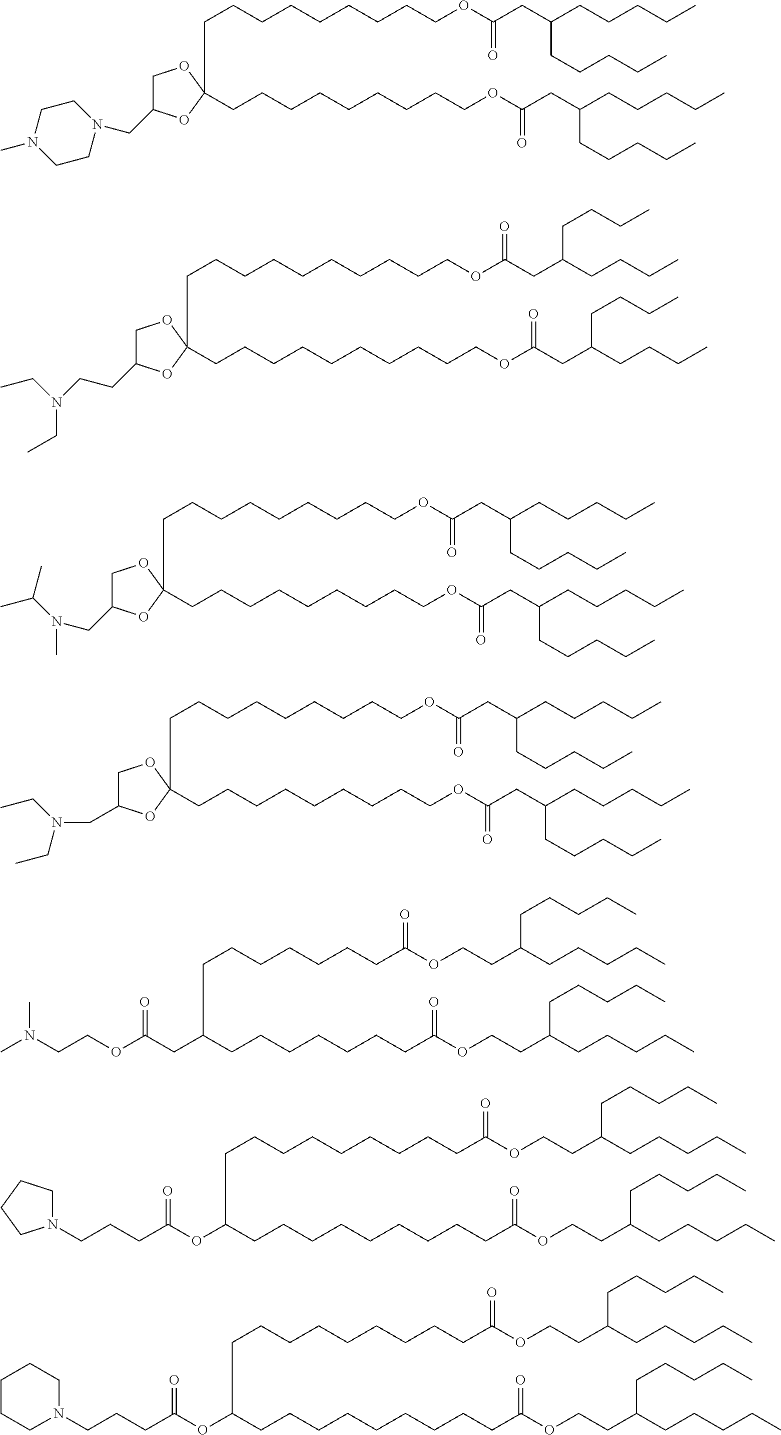

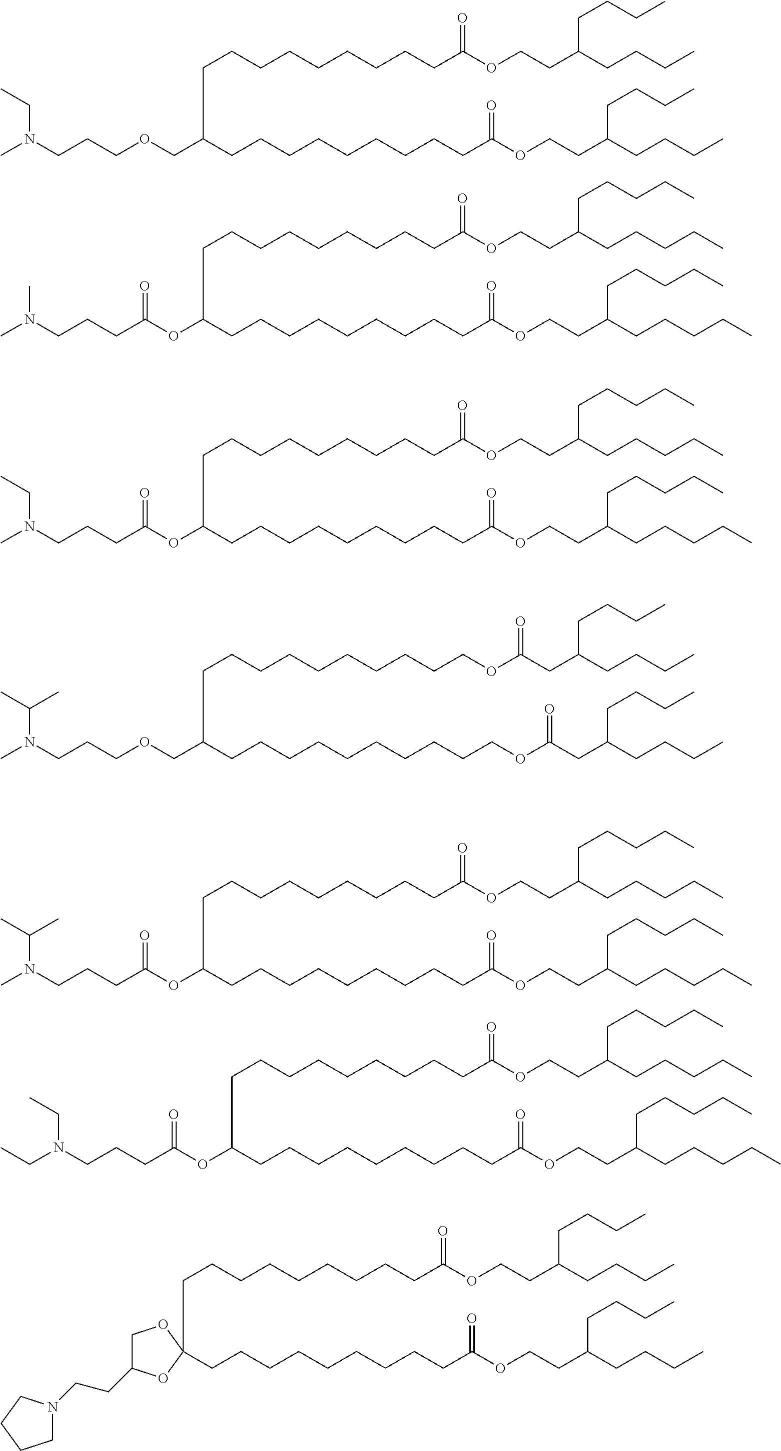

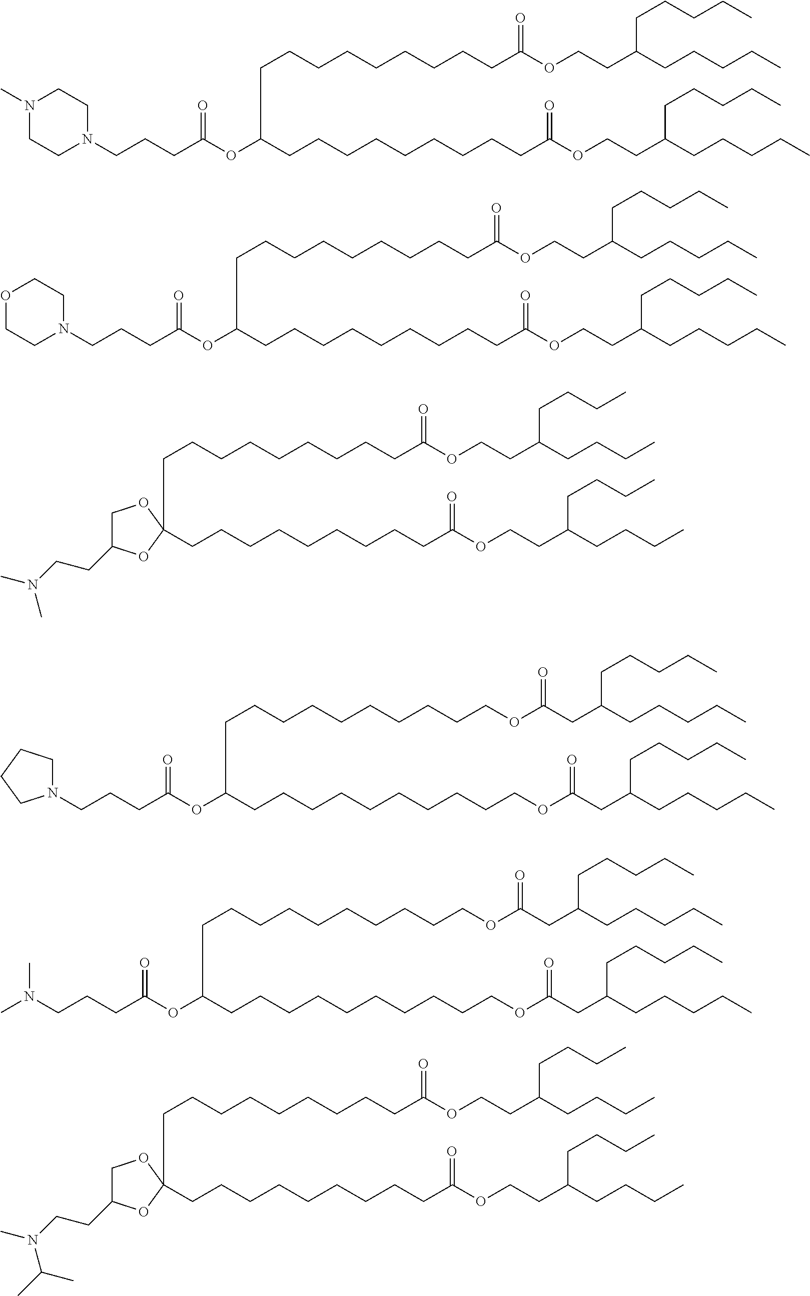

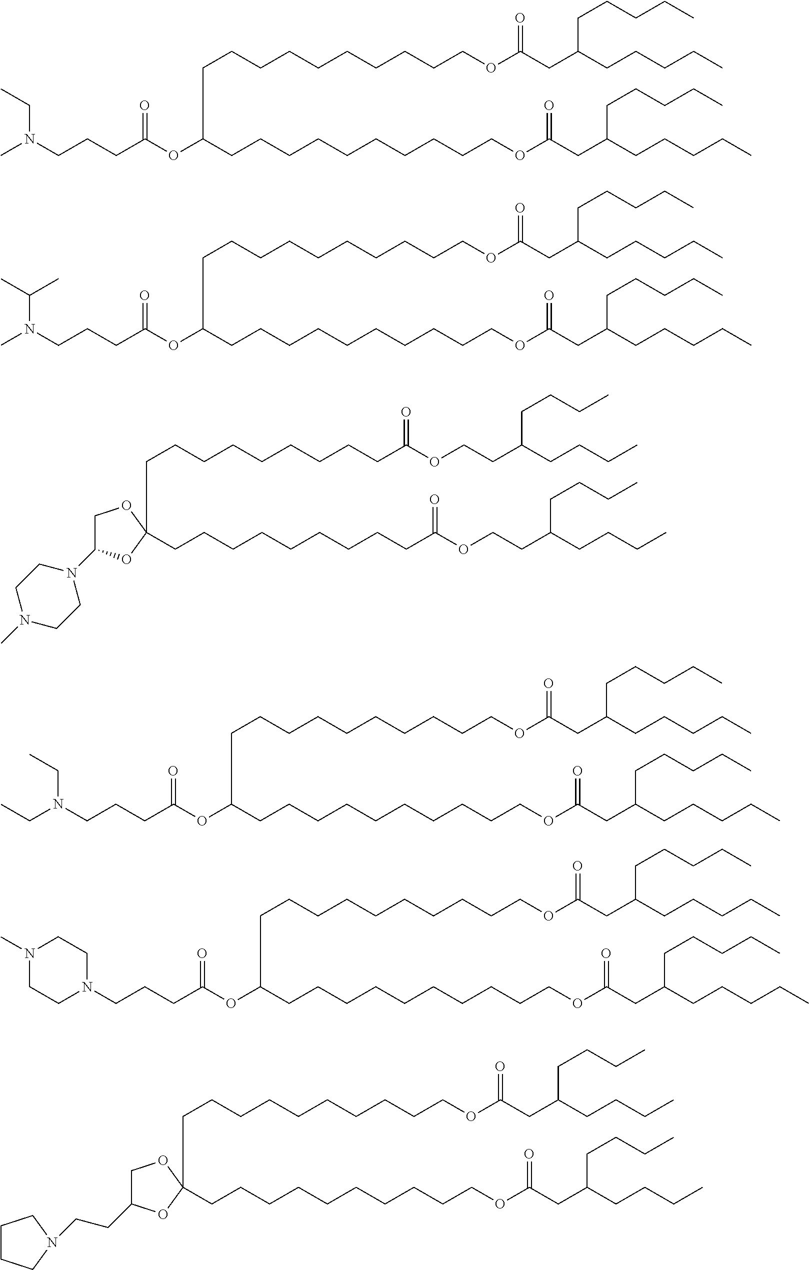

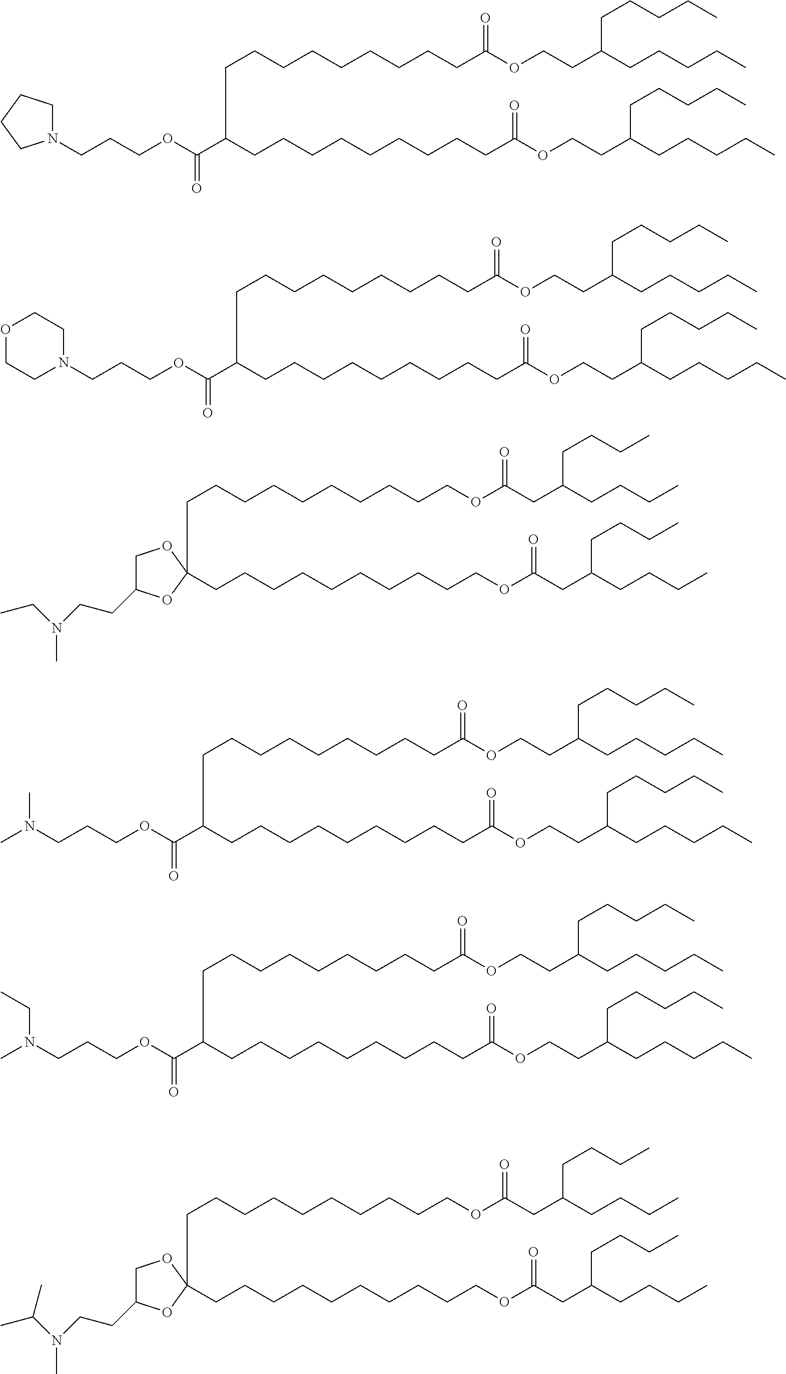

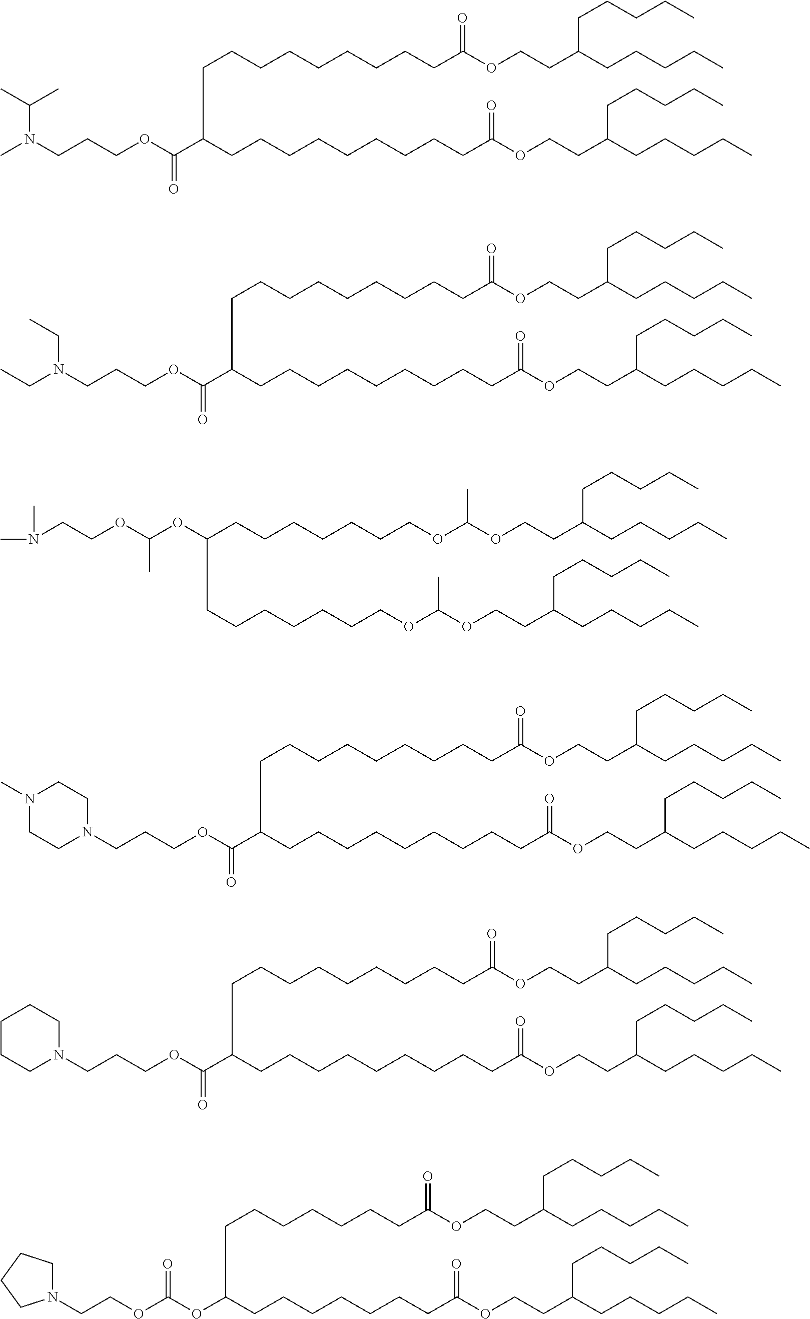

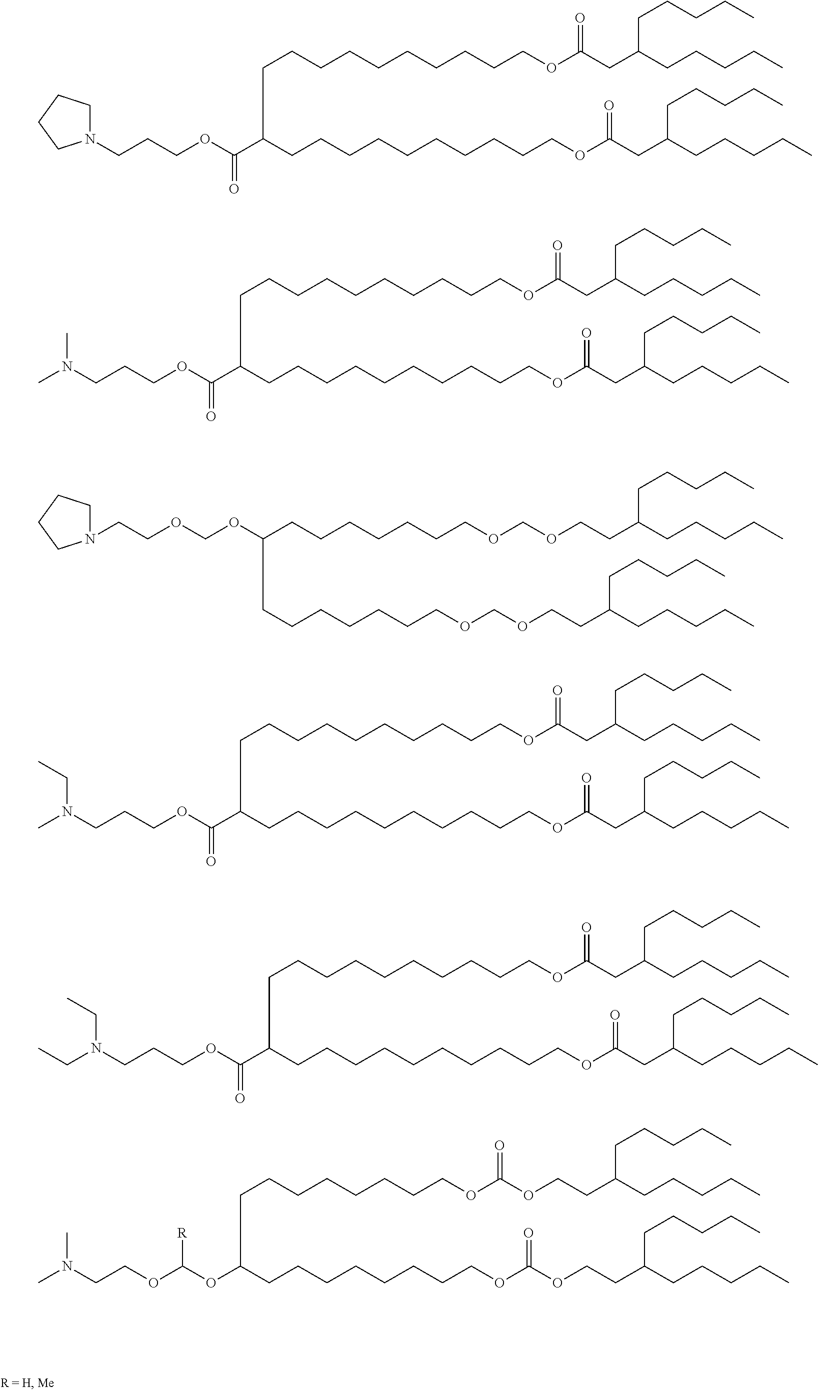

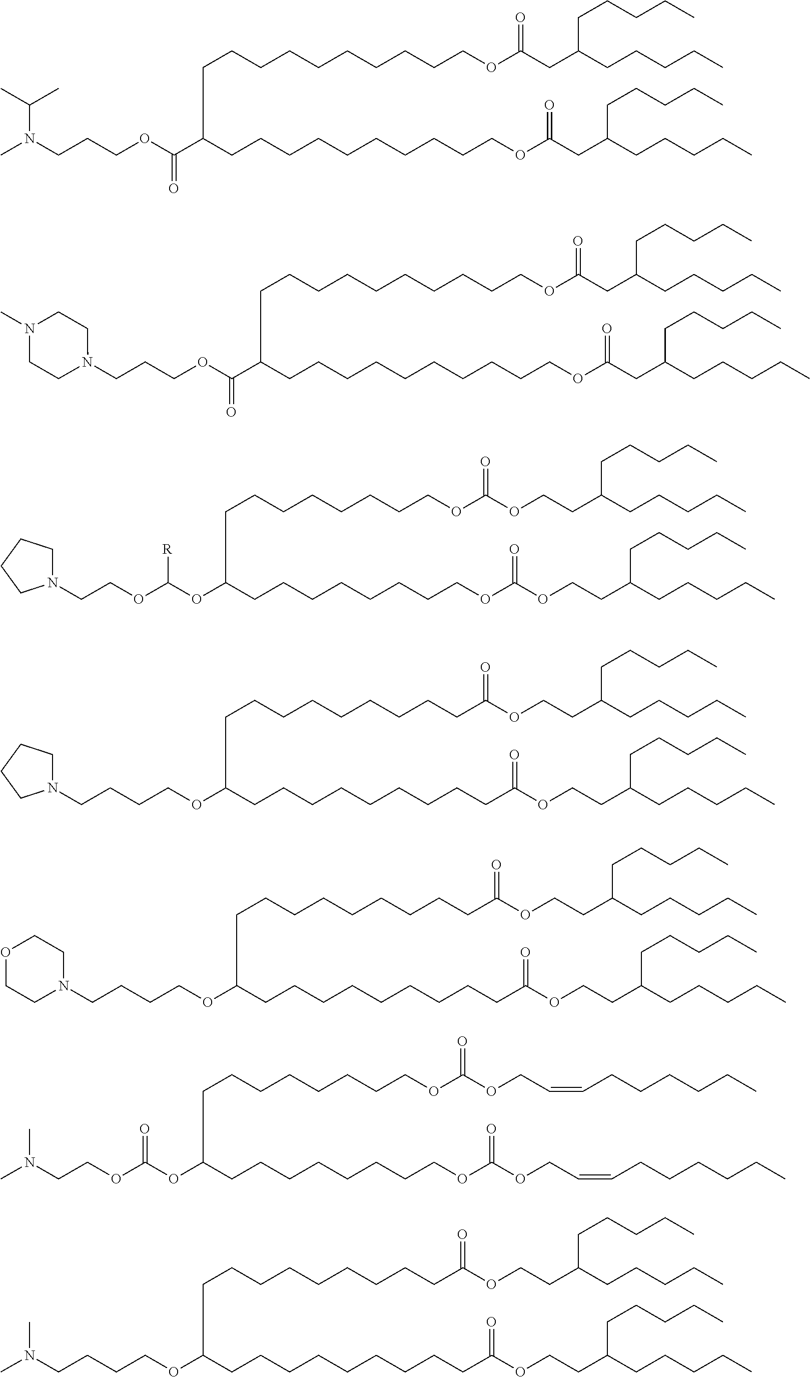

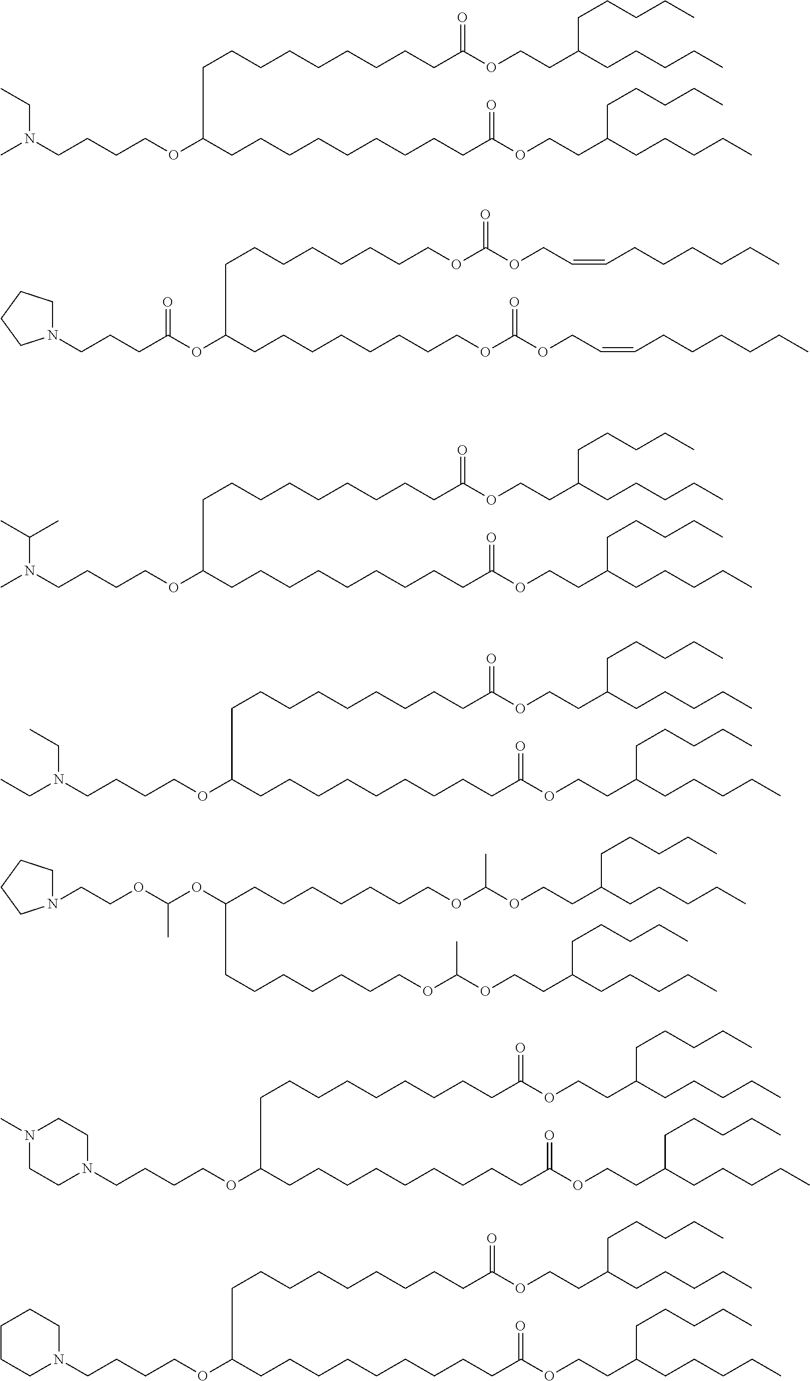

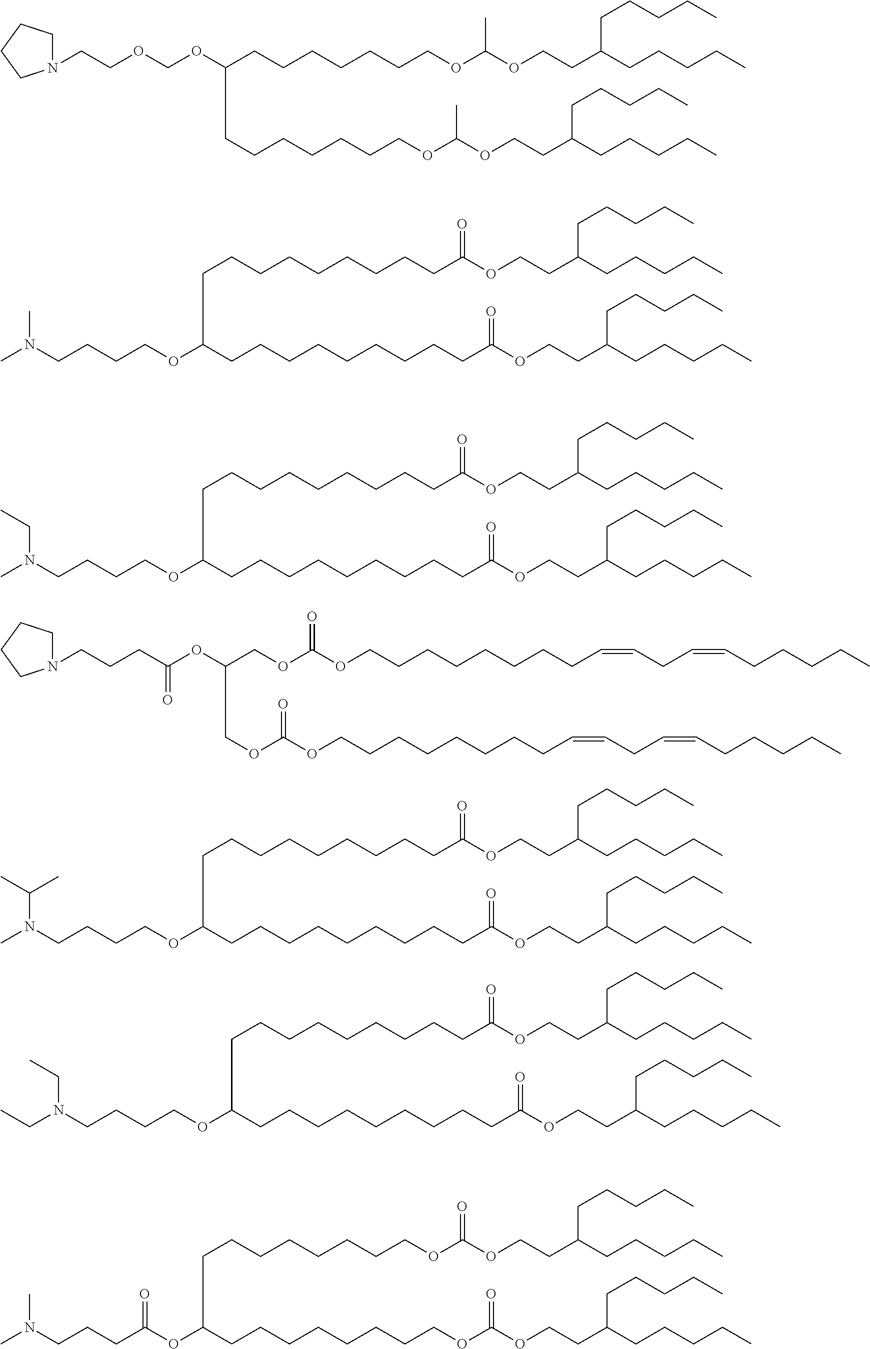

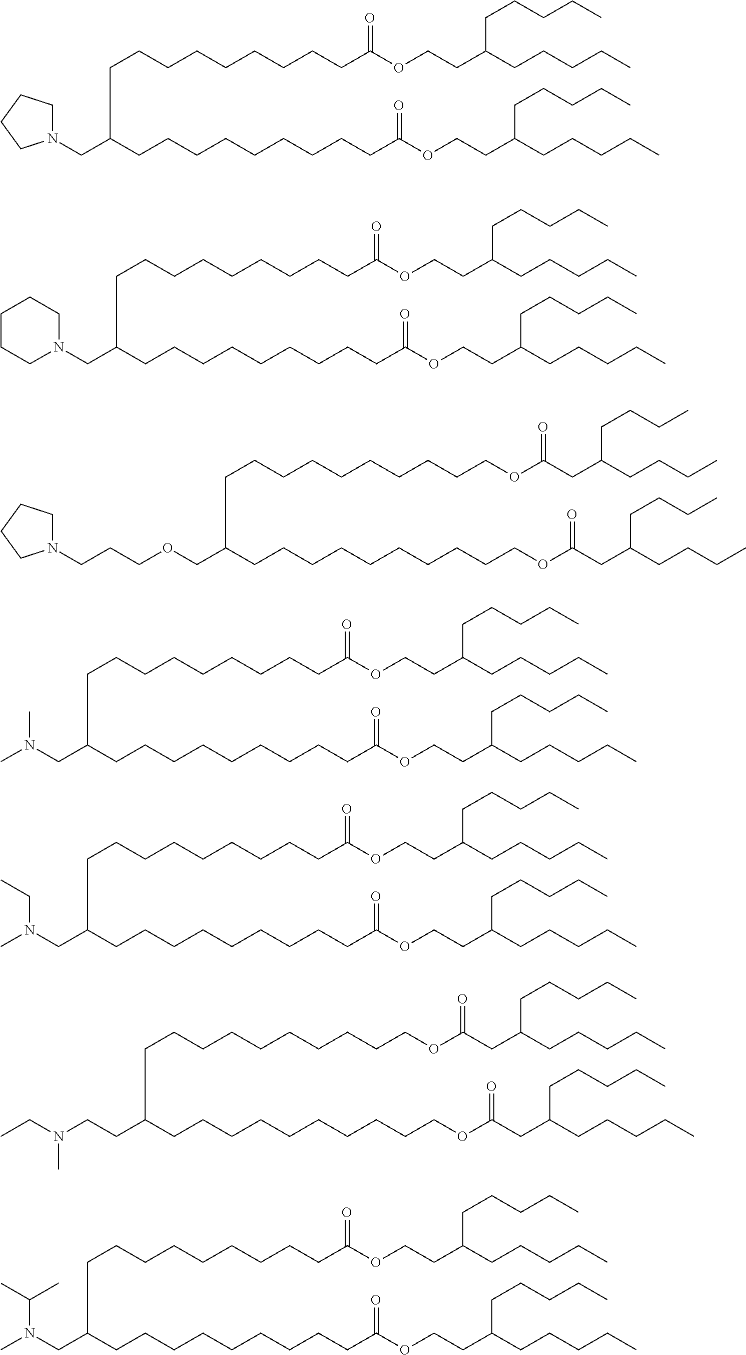

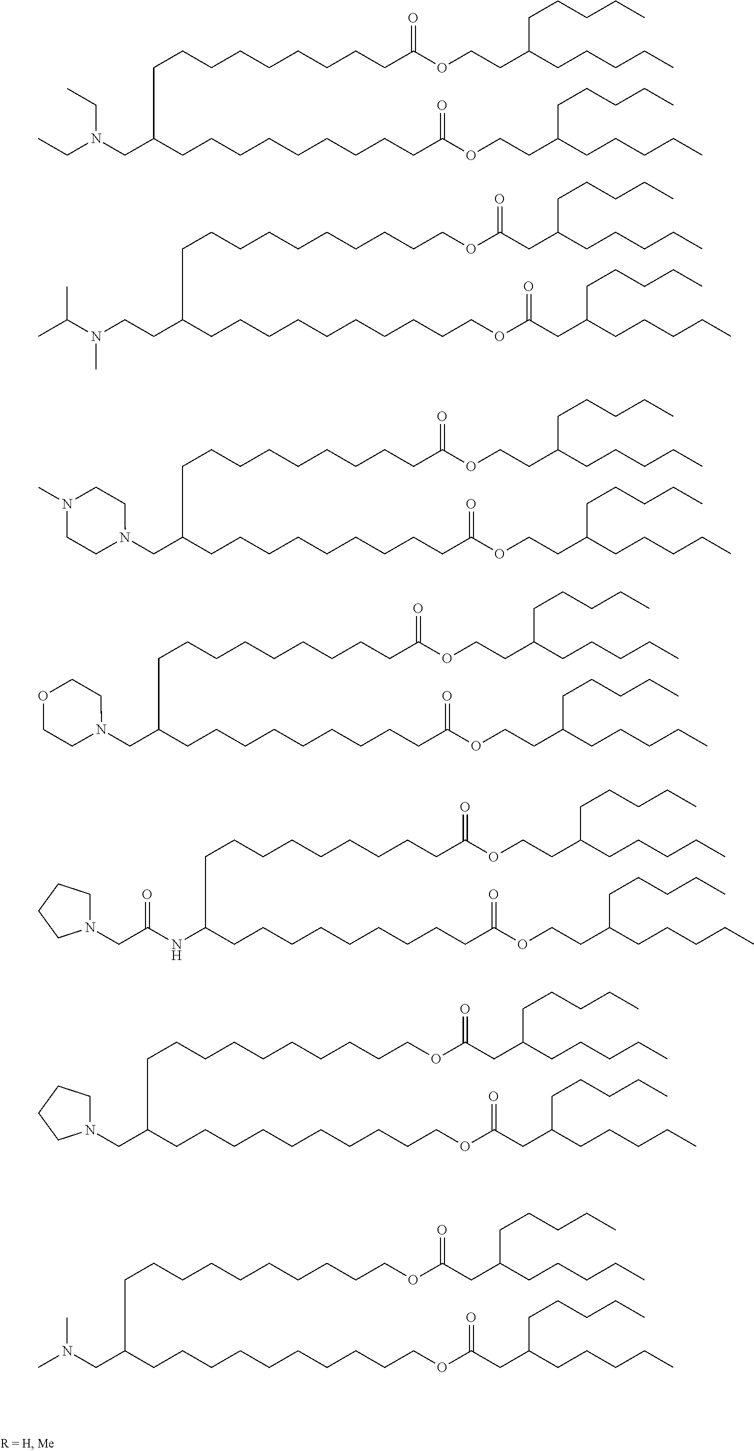

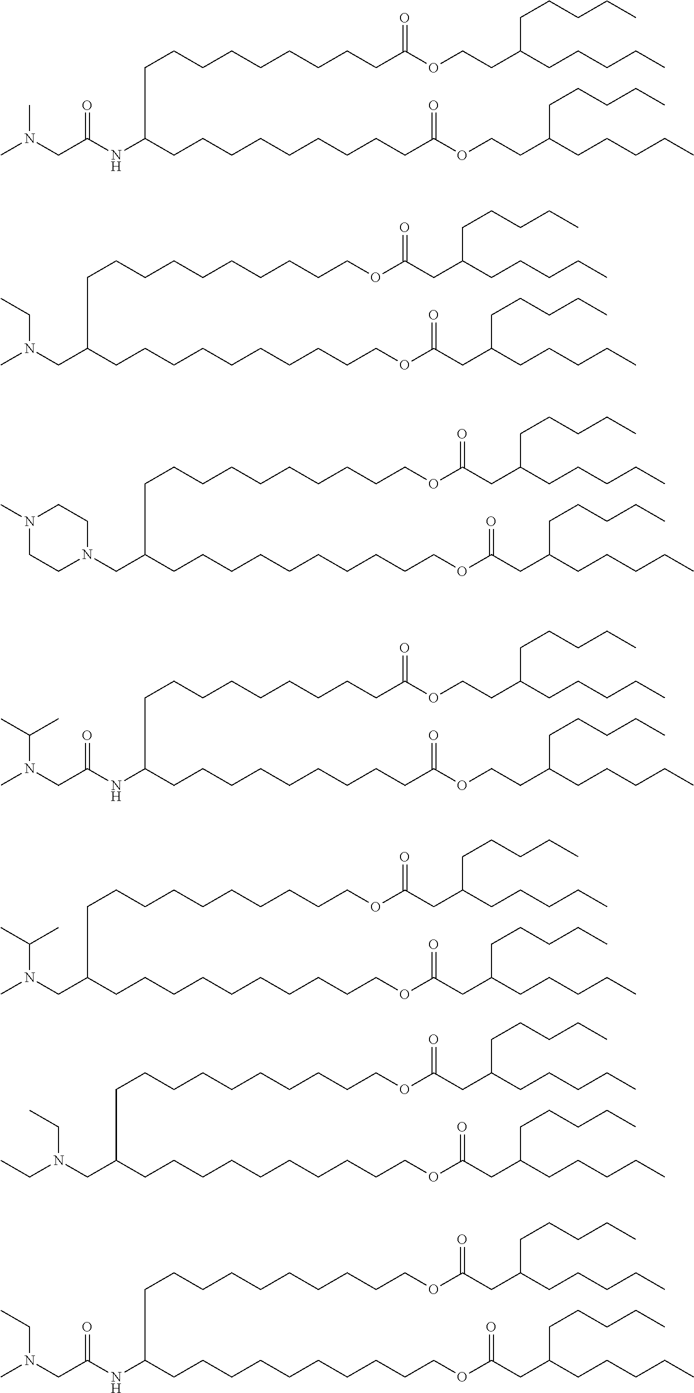

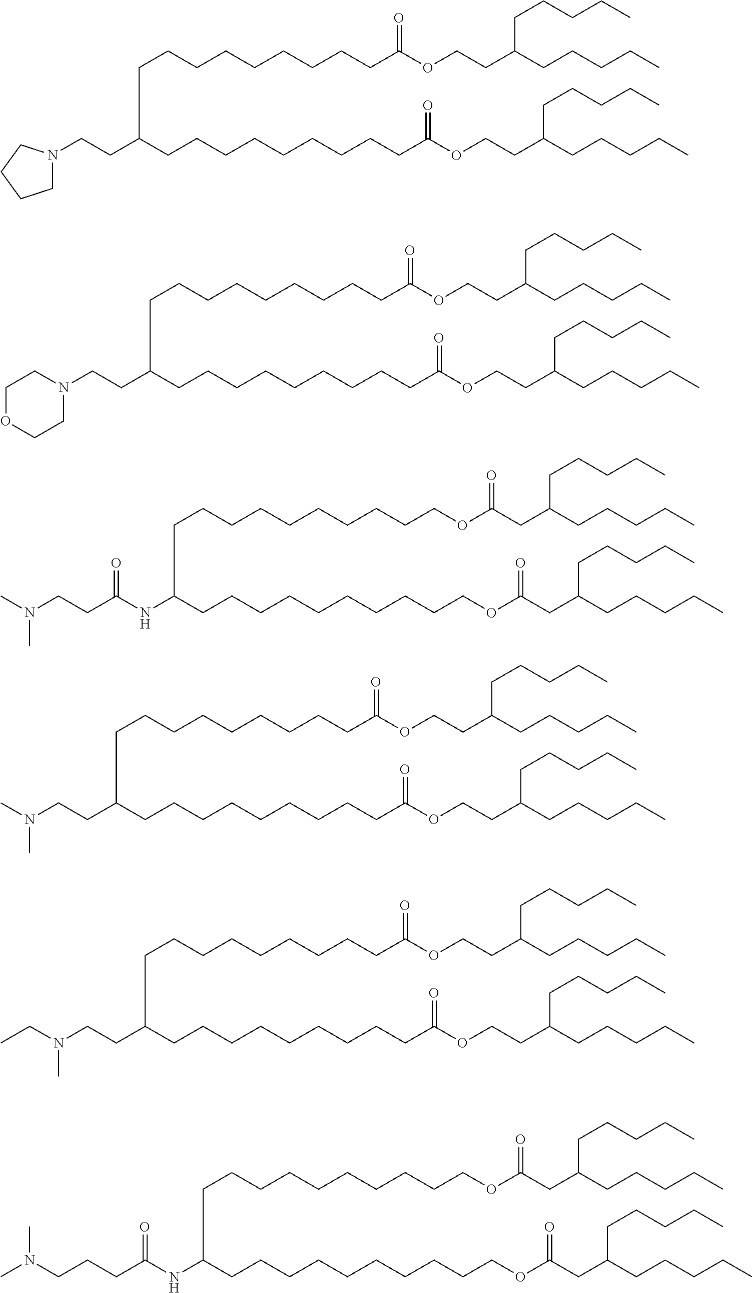

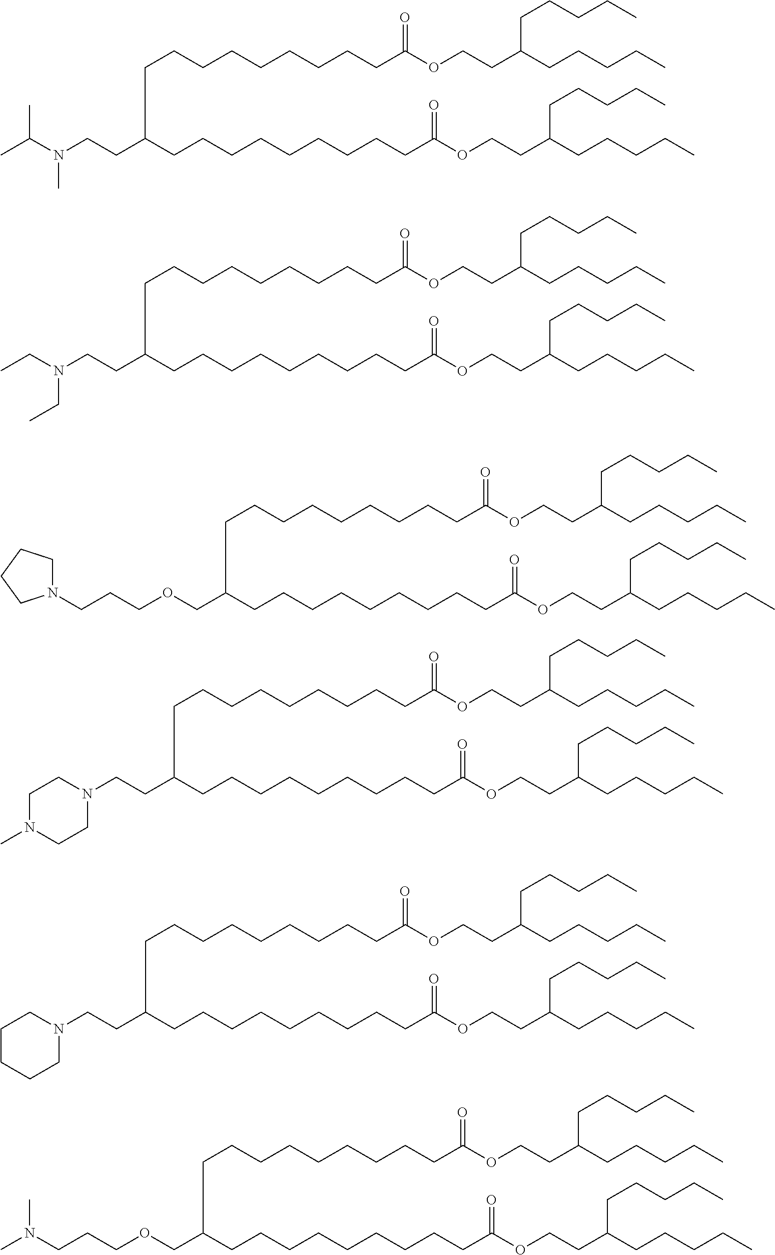

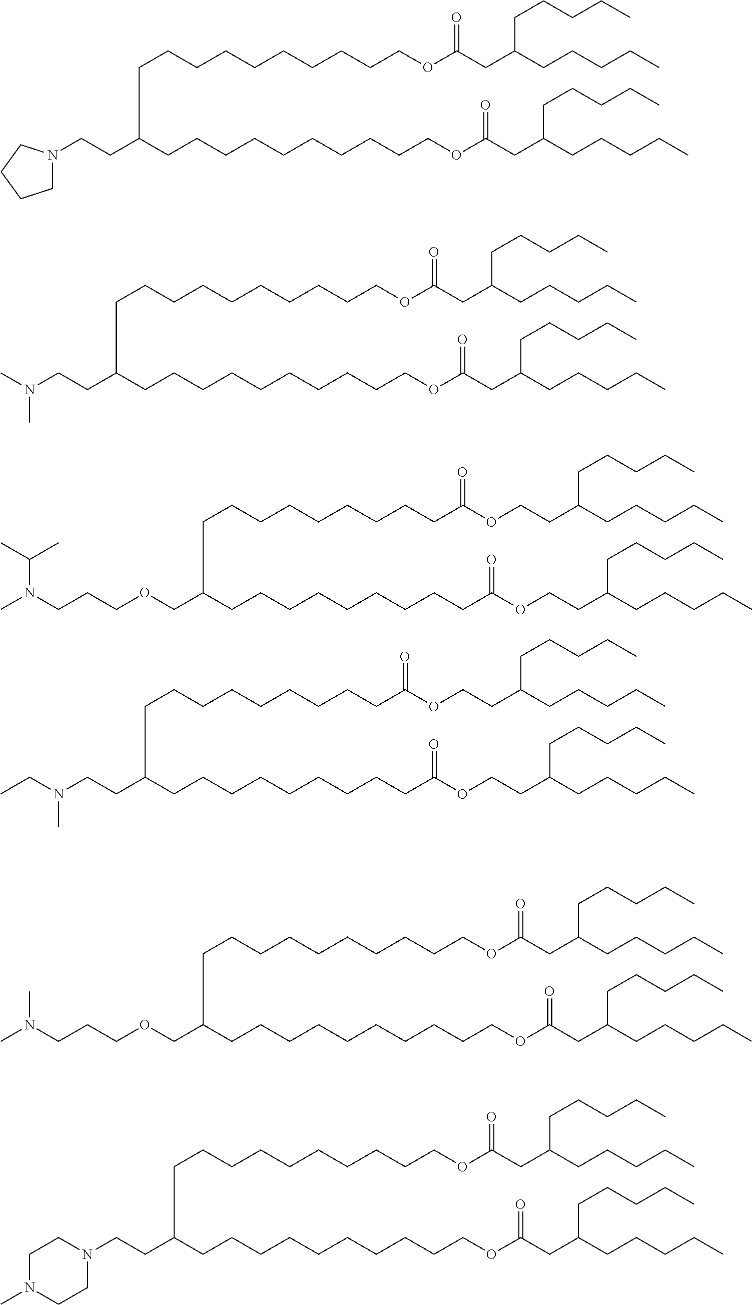

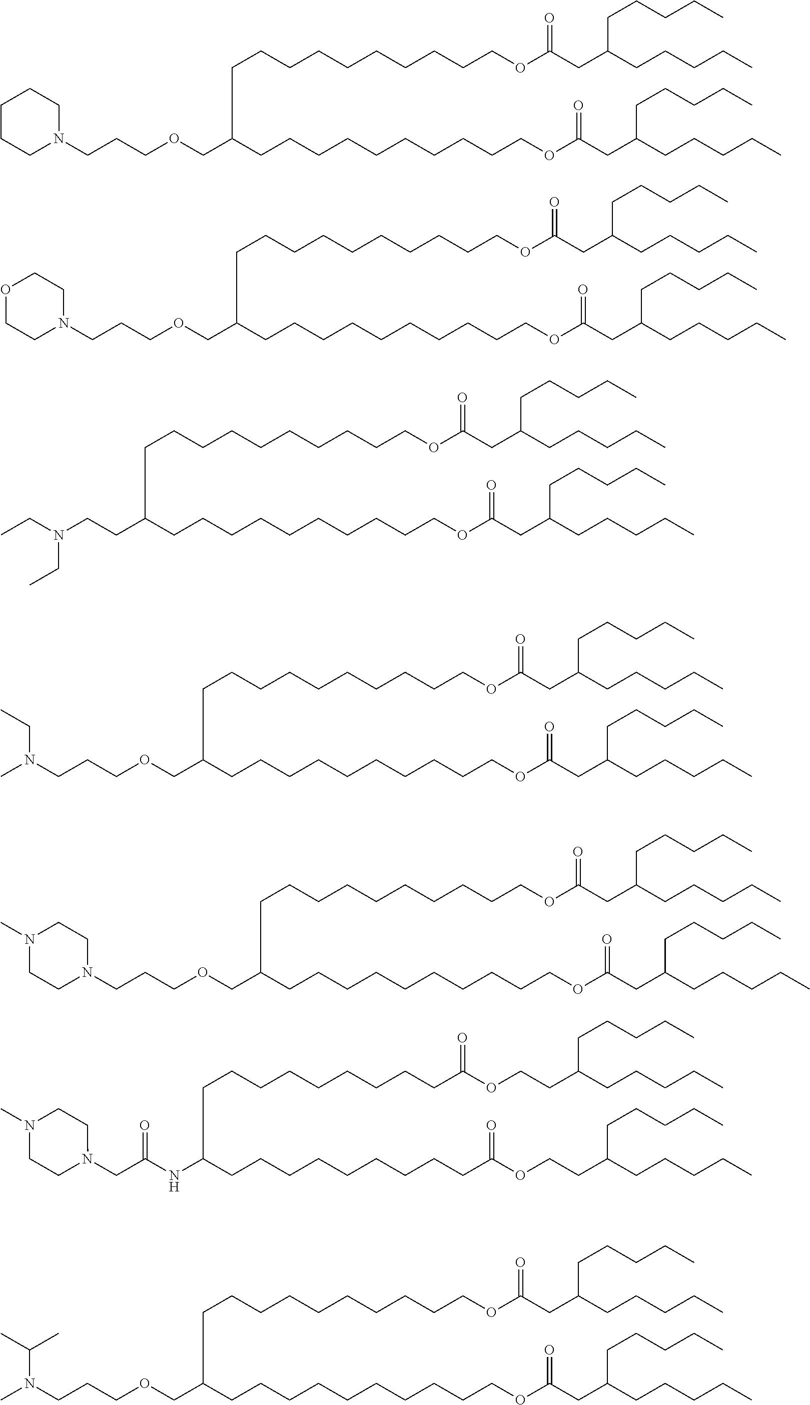

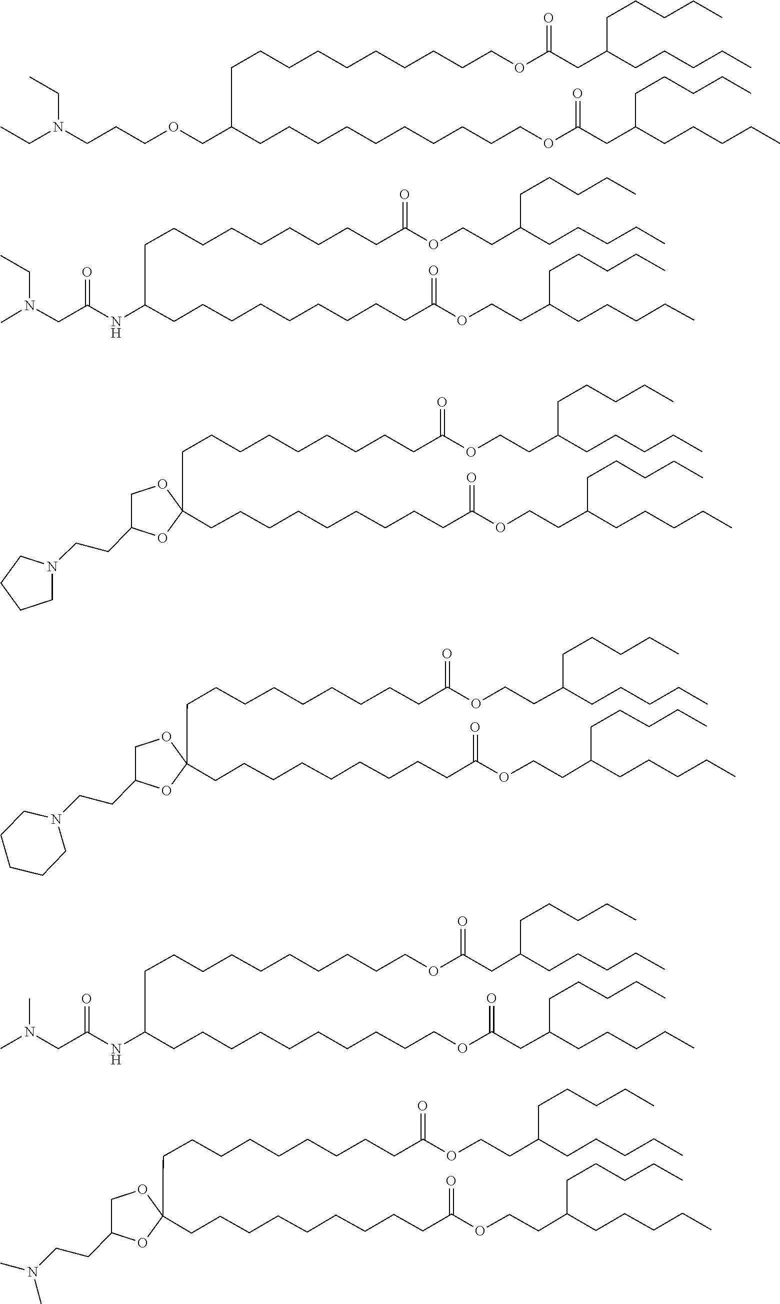

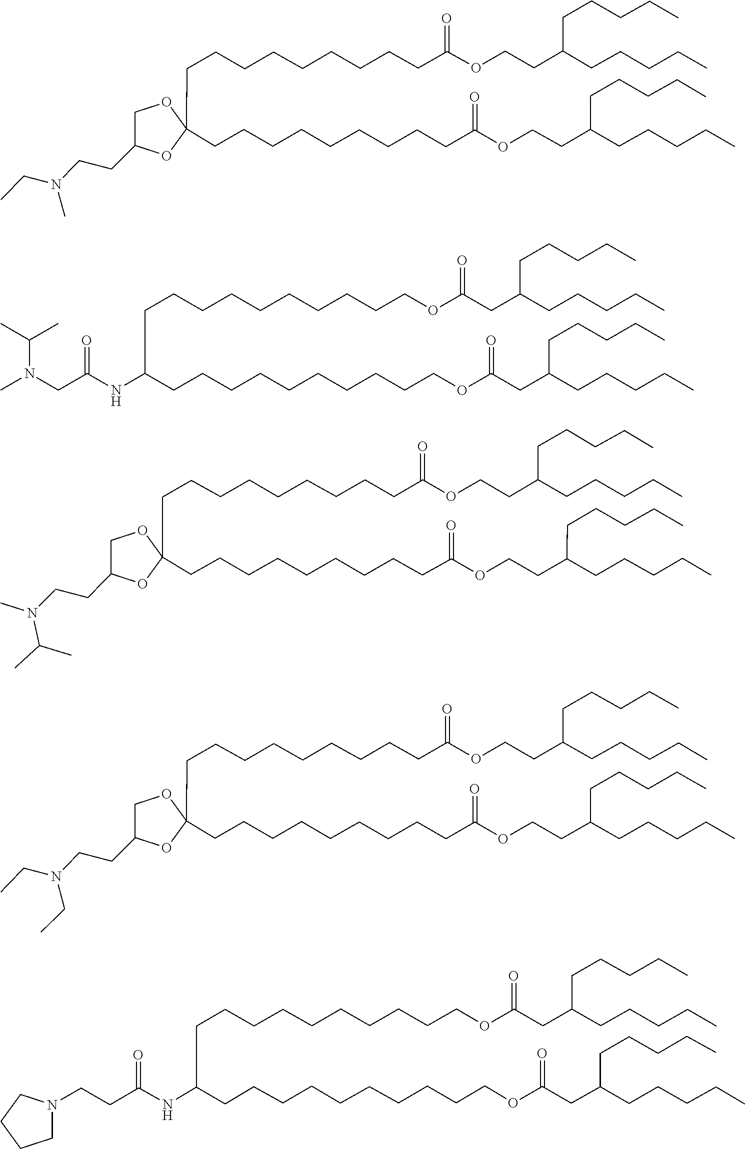

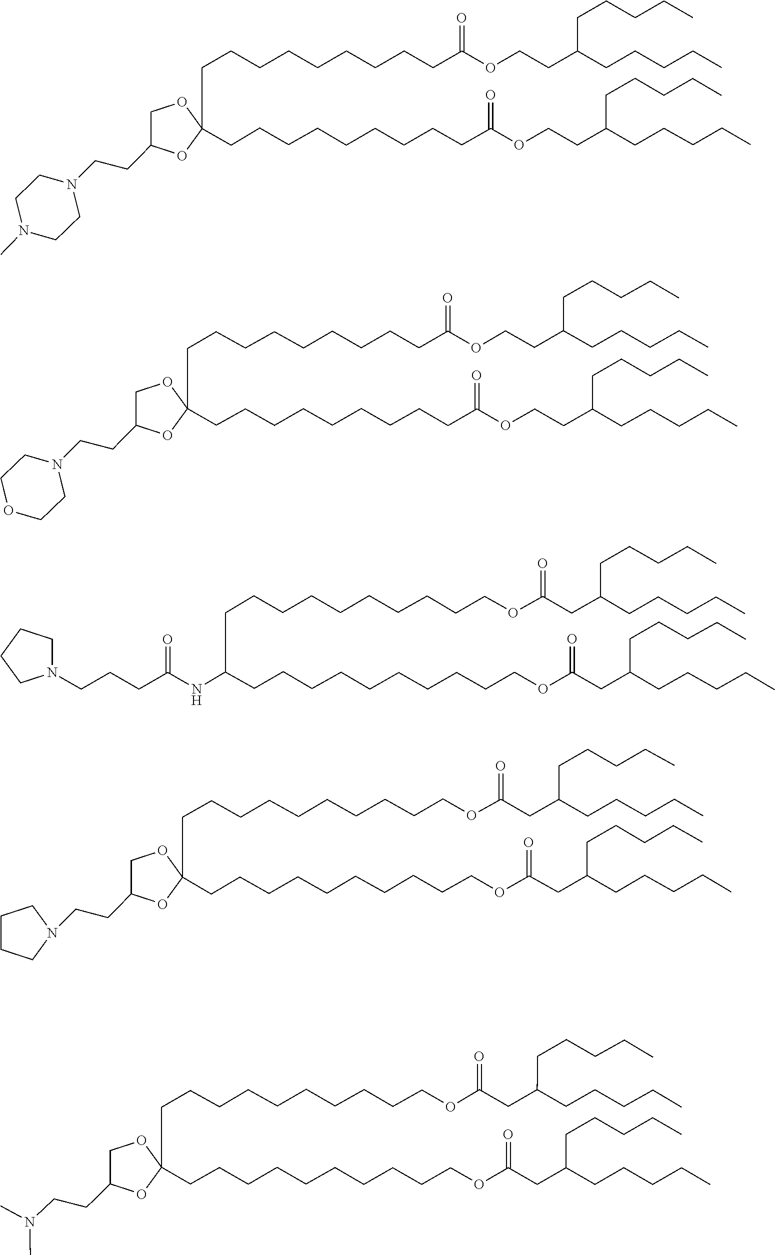

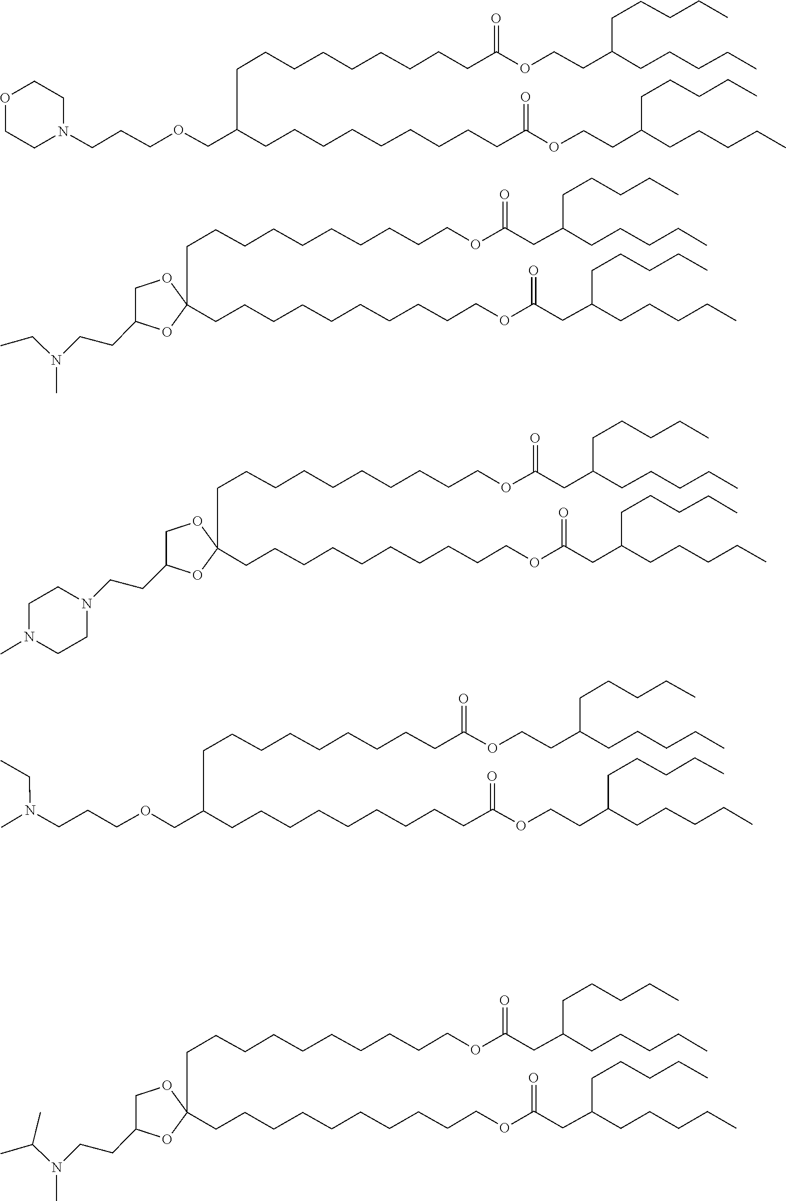

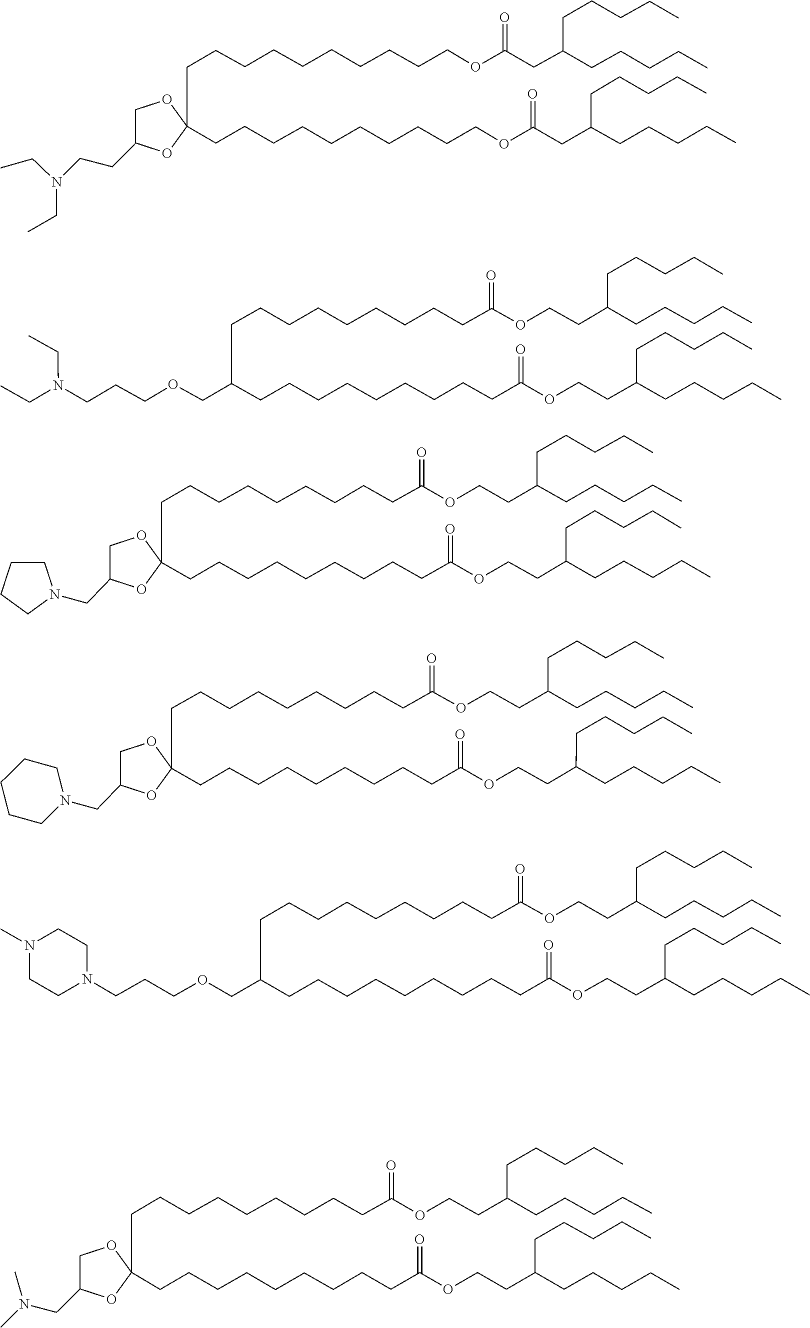

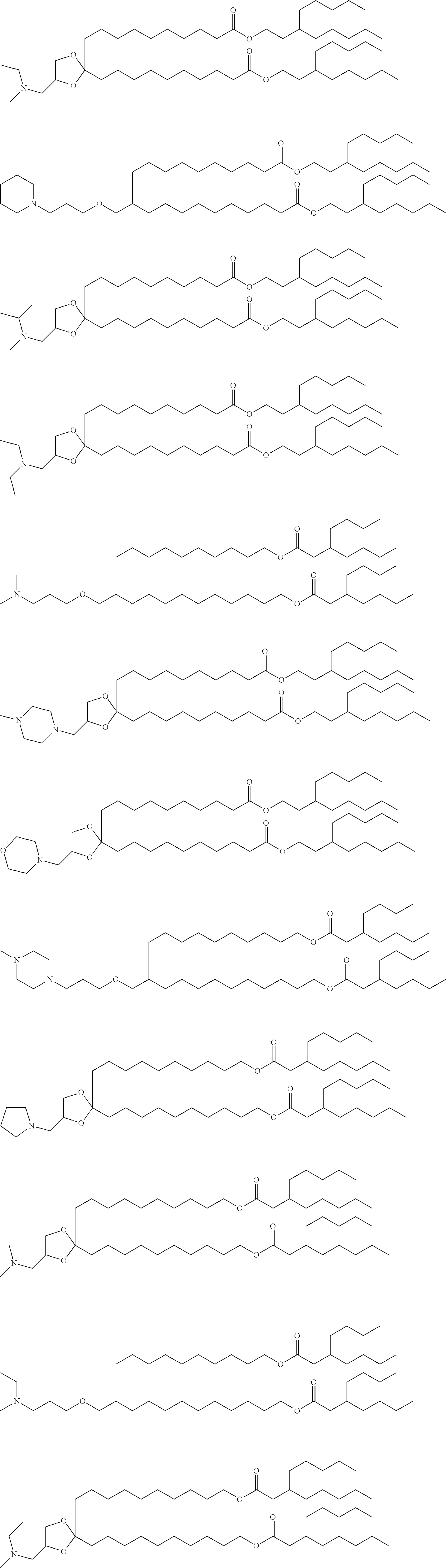

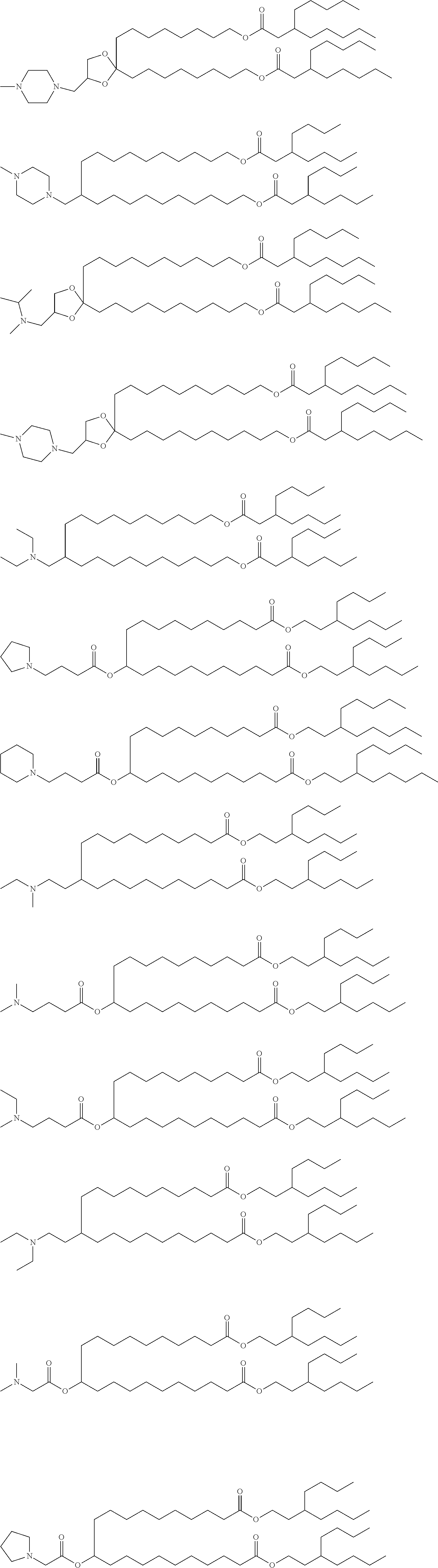

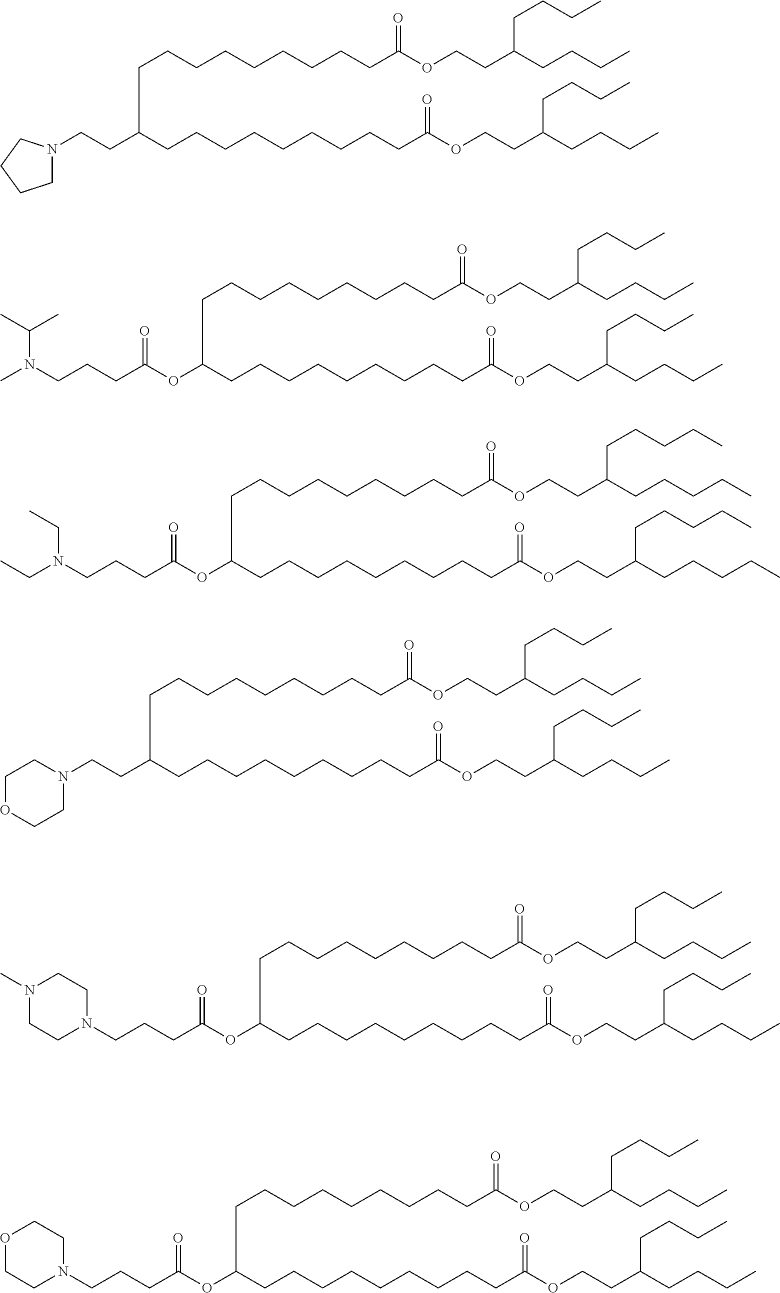

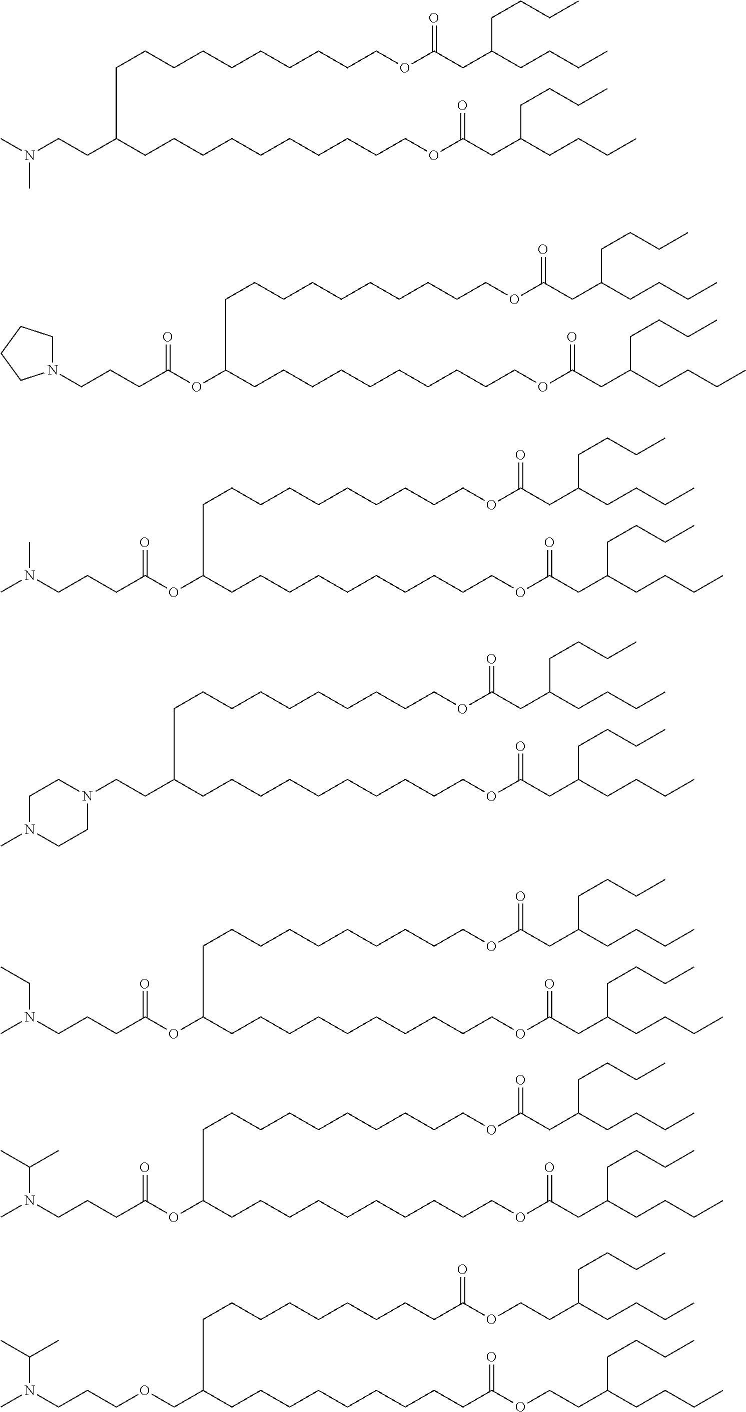

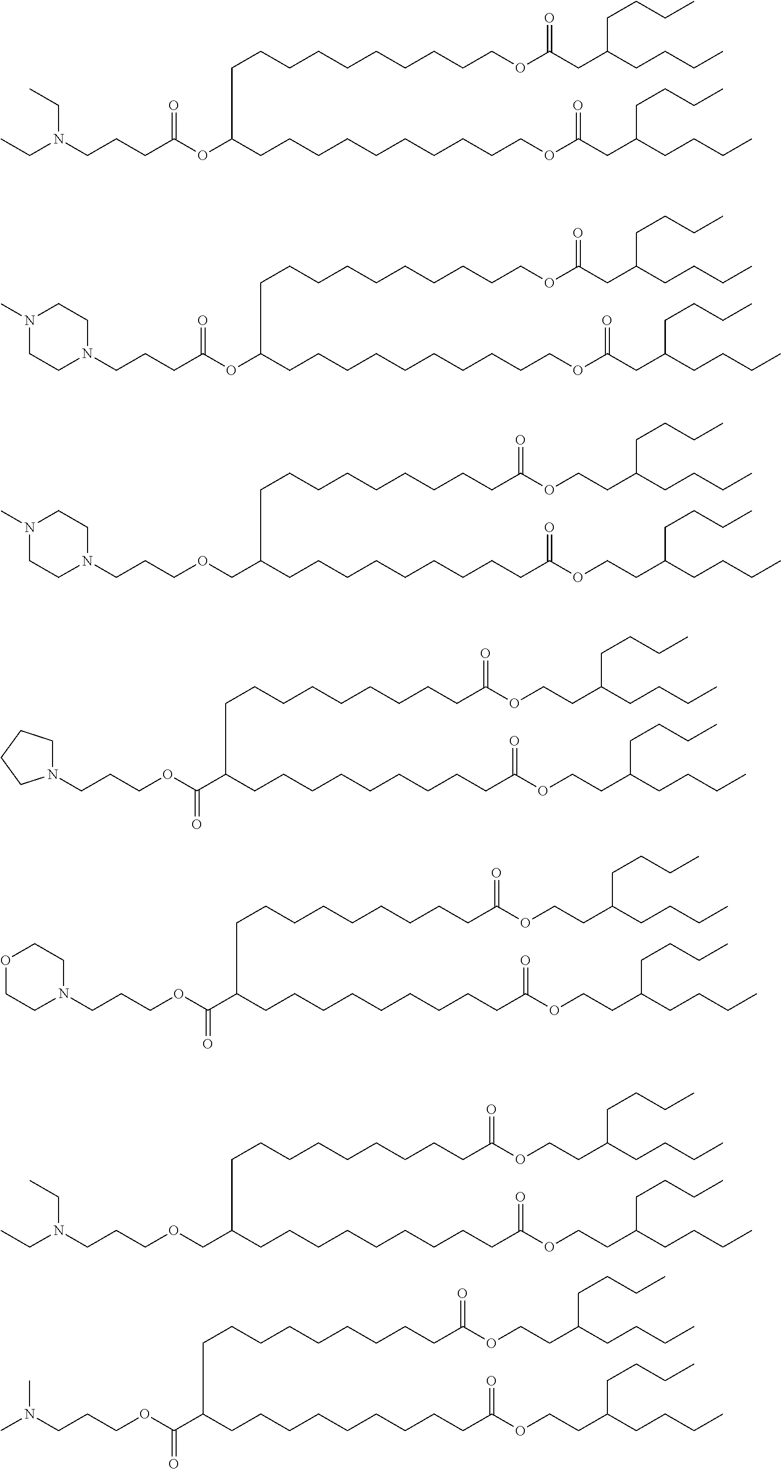

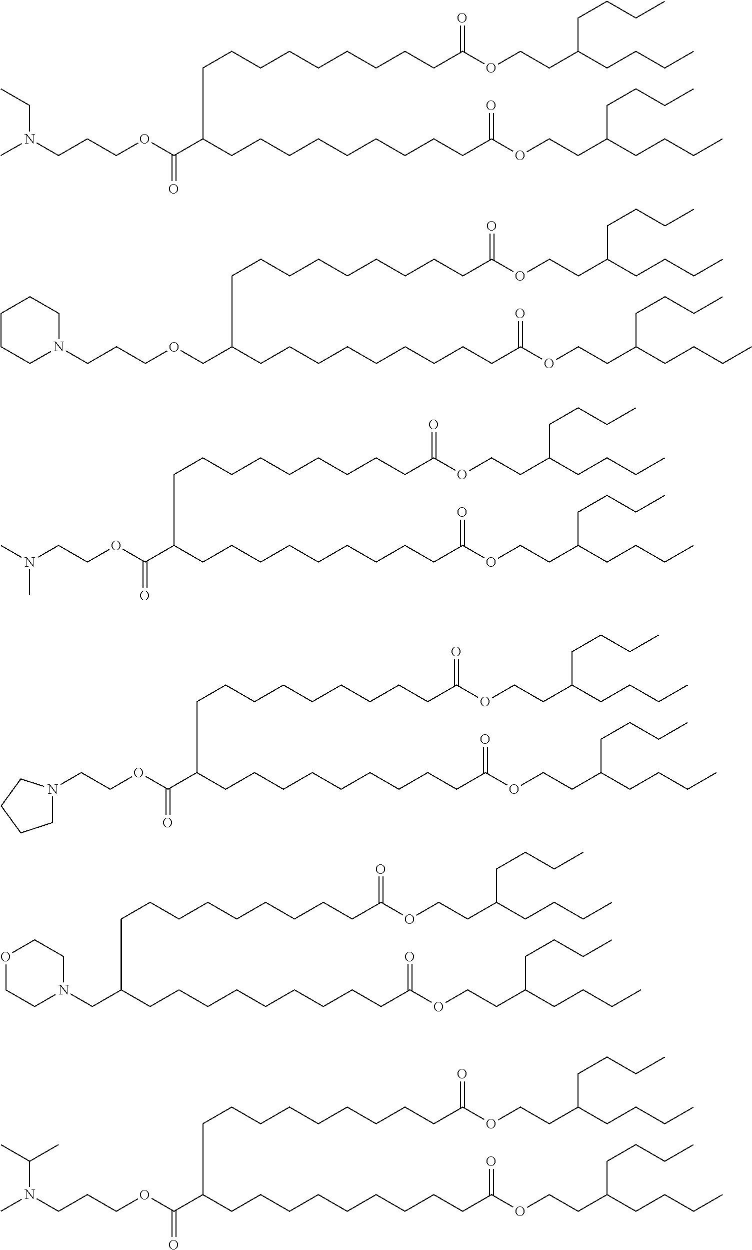

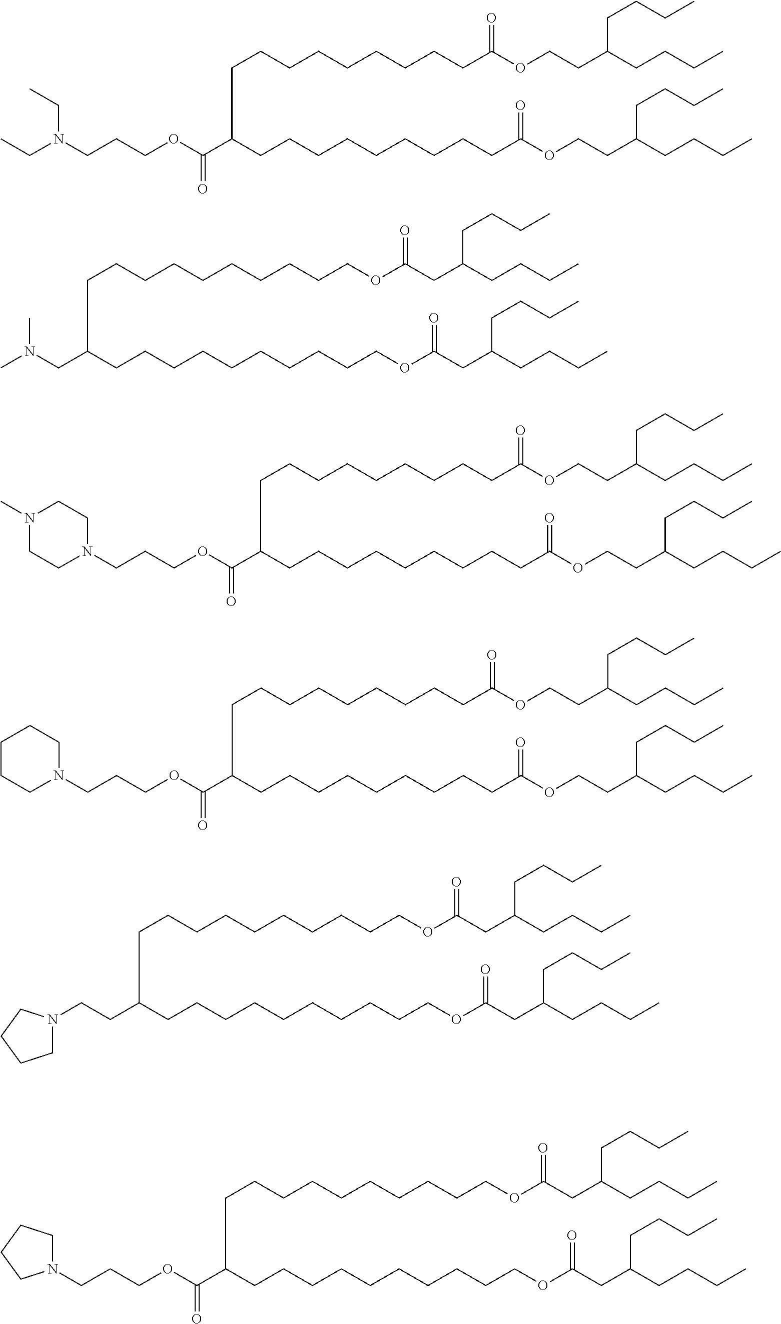

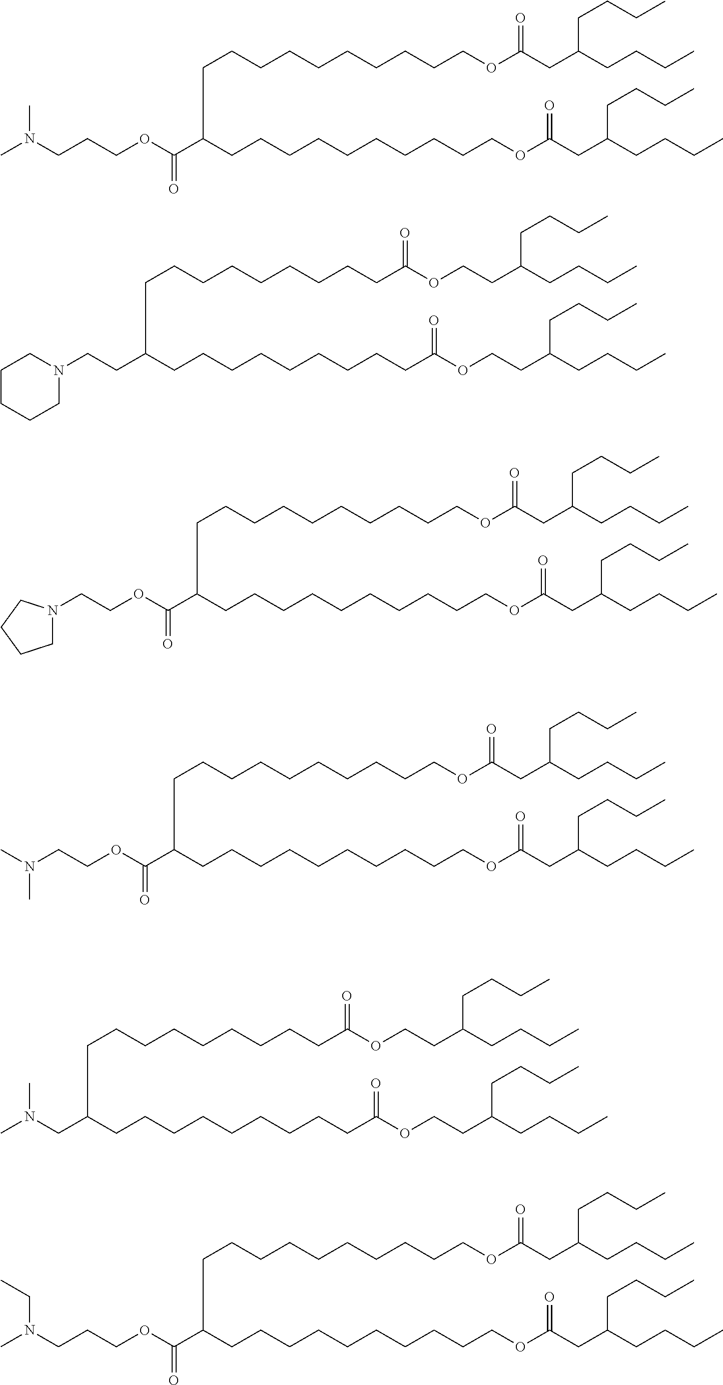

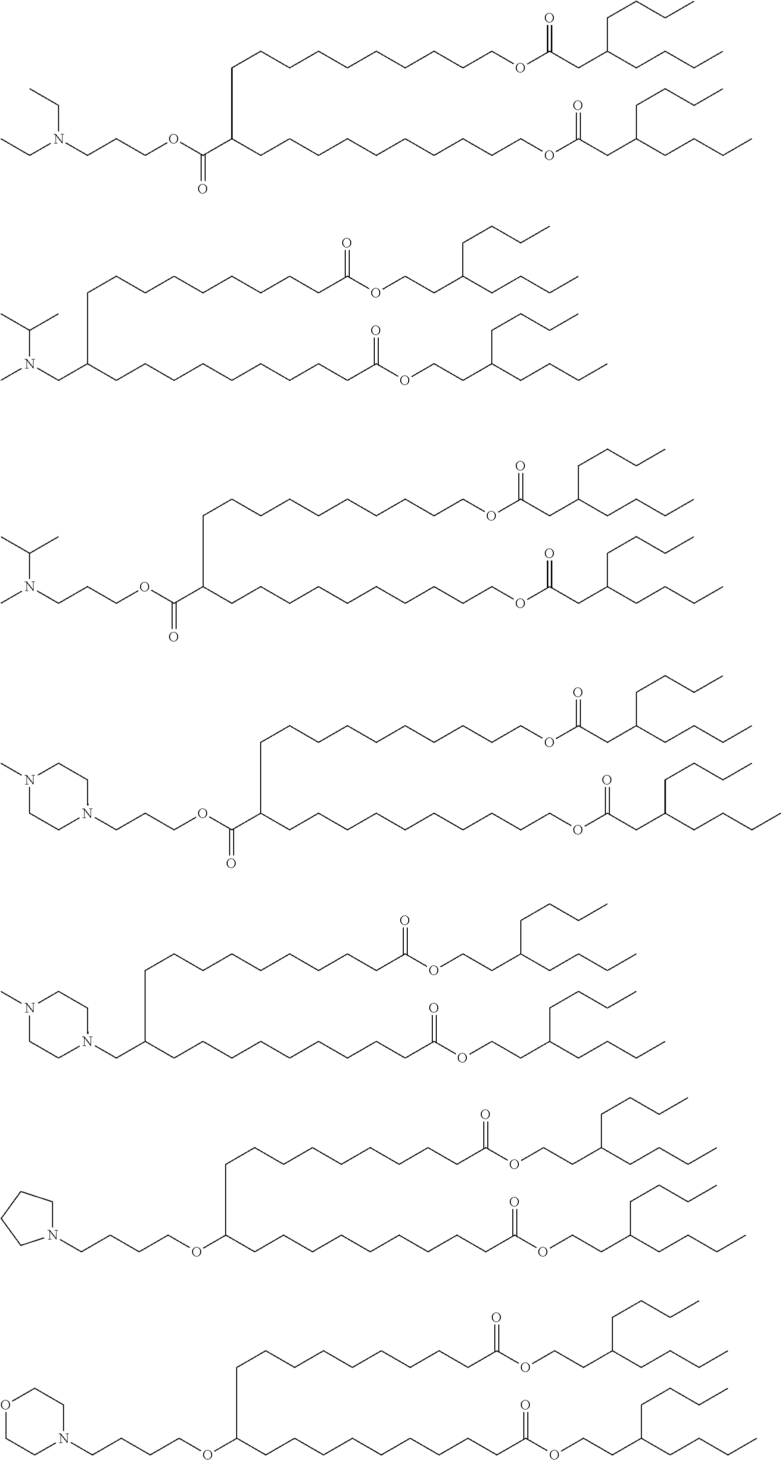

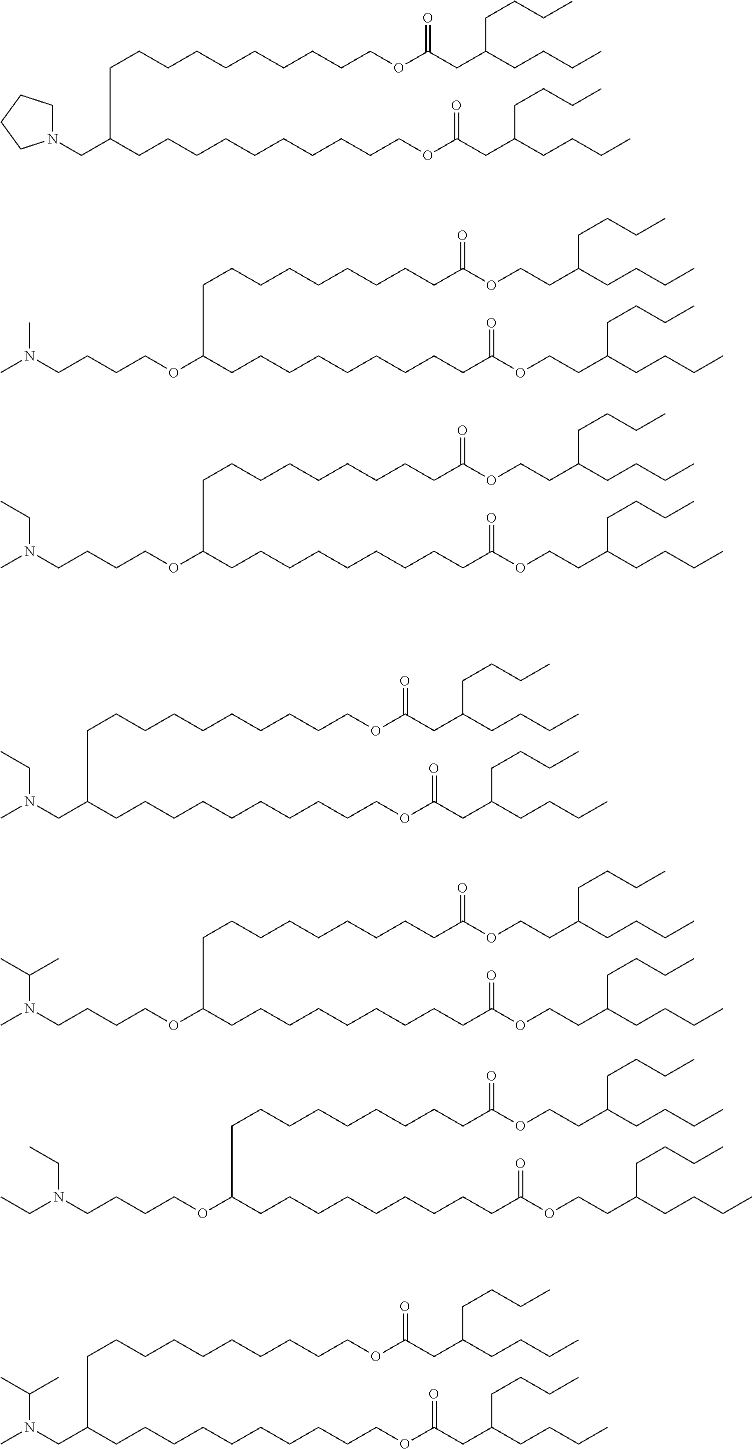

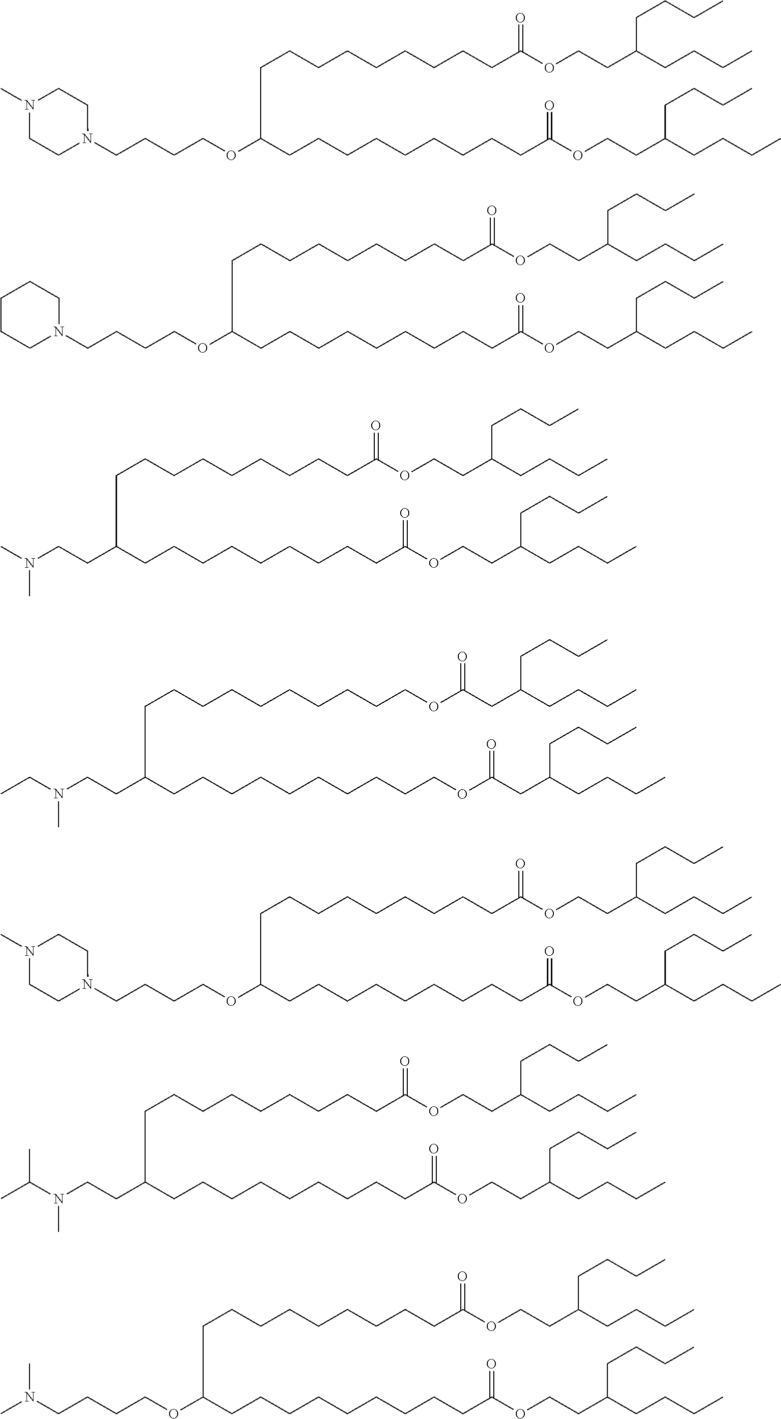

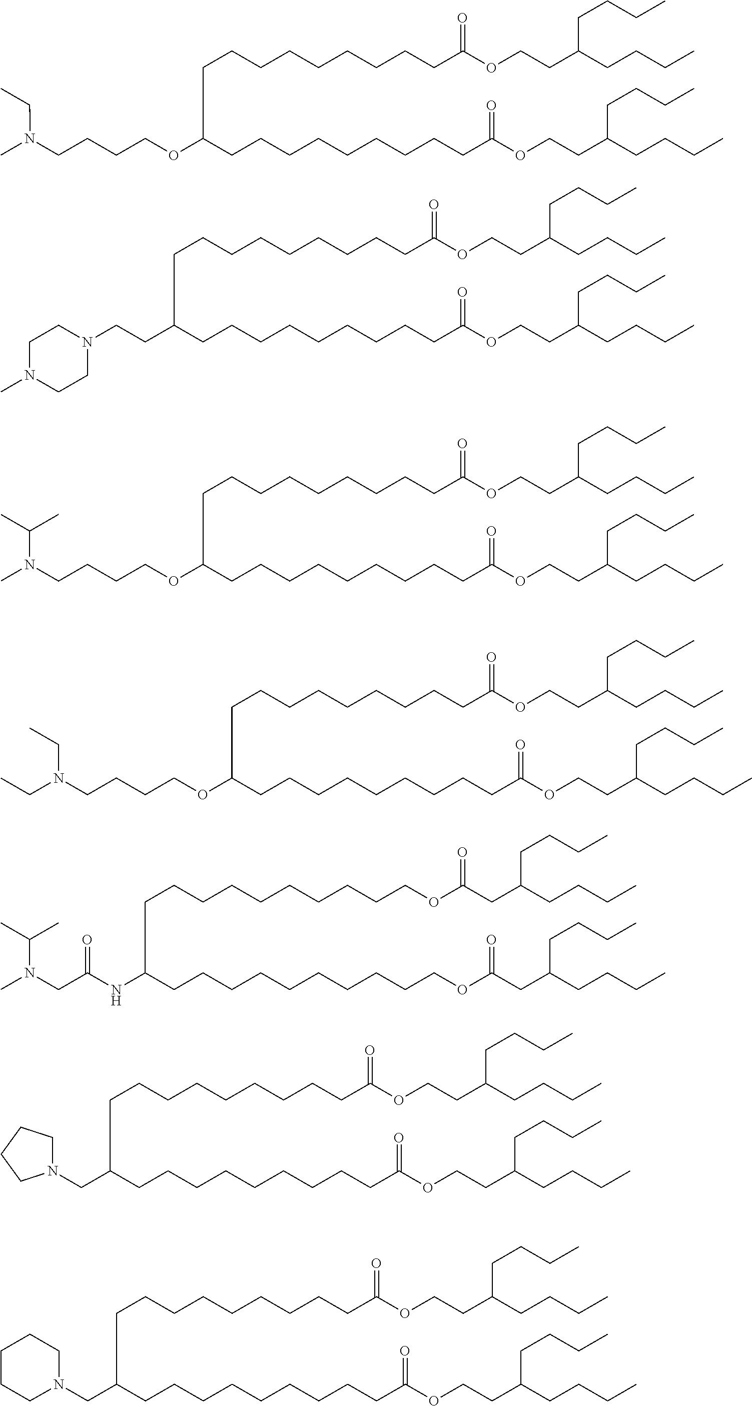

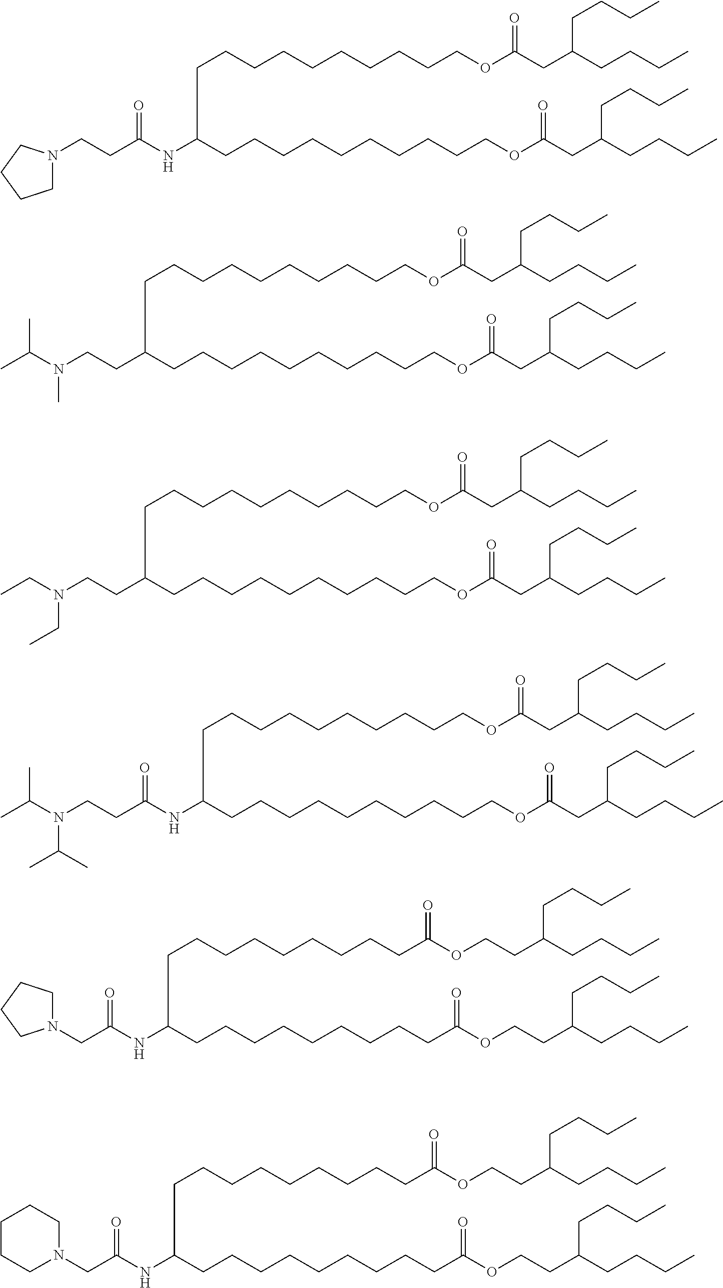

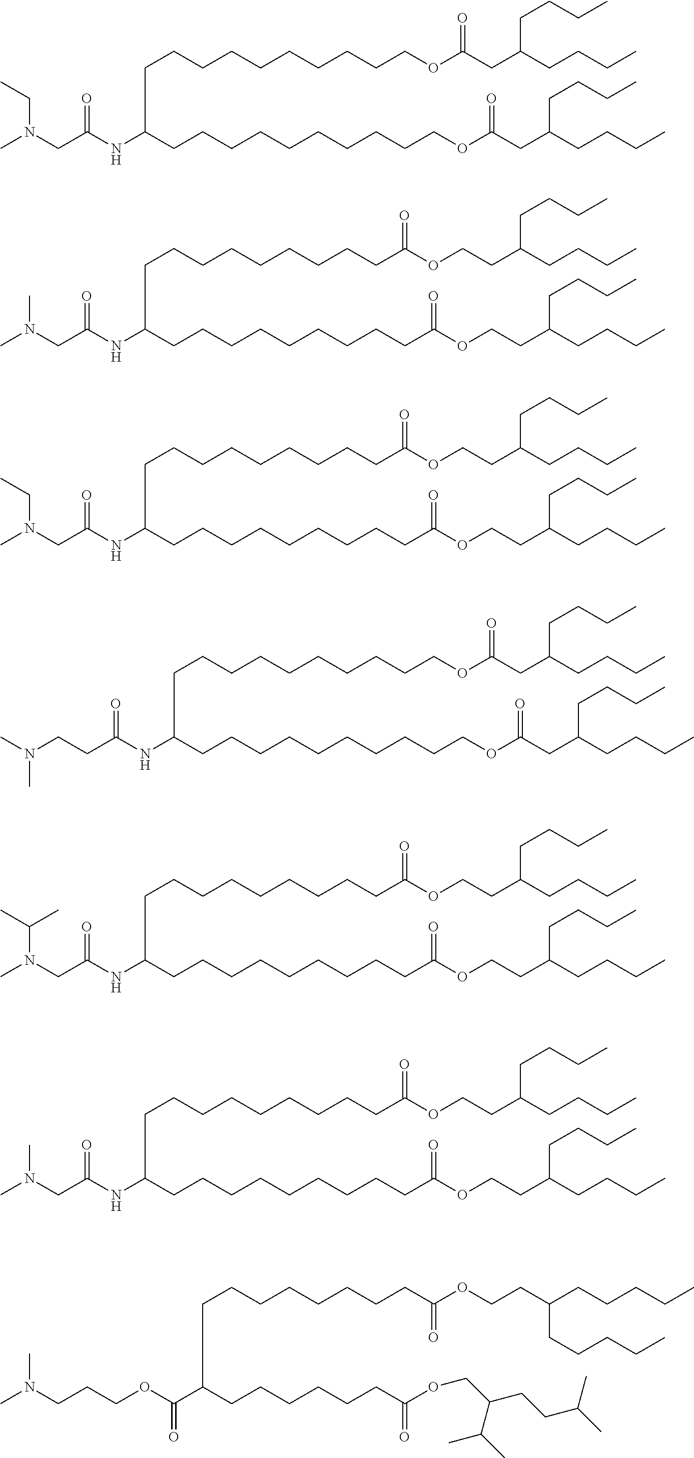

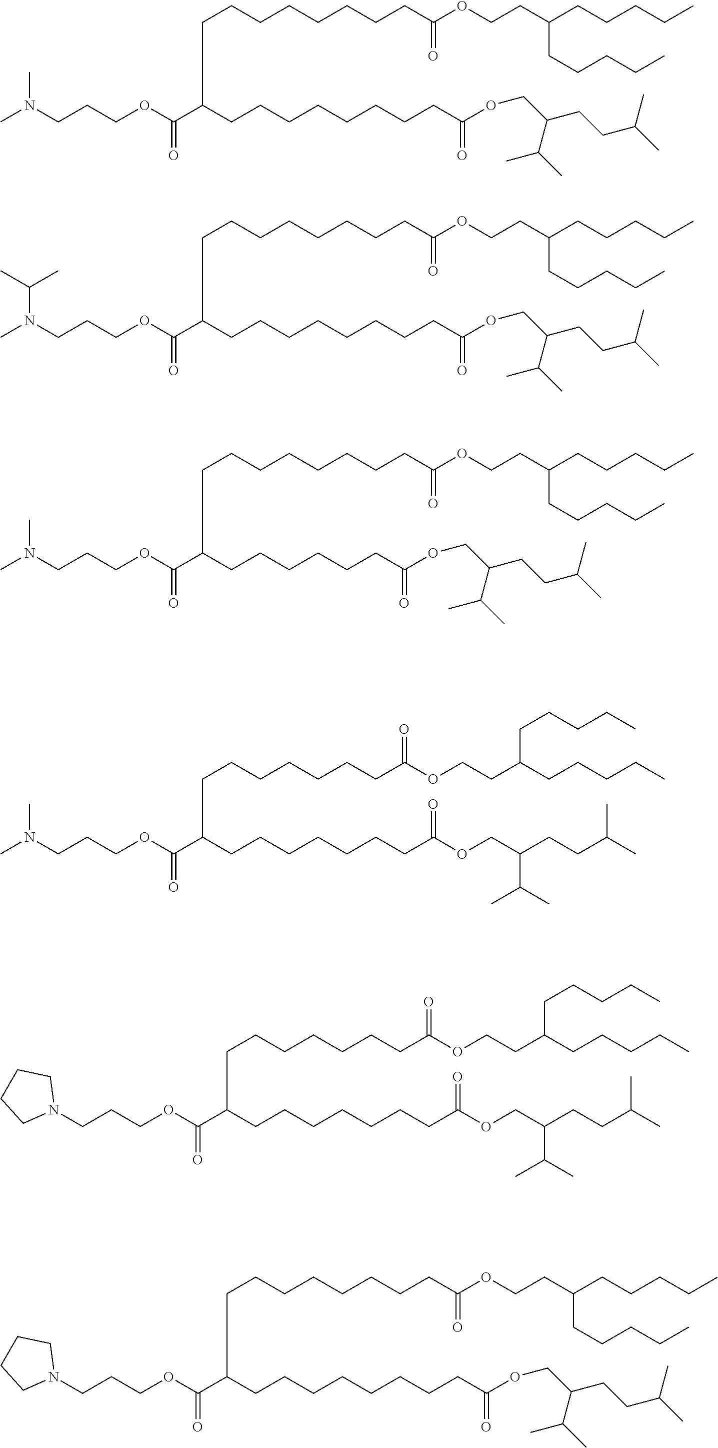

In some embodiments, the cationic lipid of the present disclosure is selected from the following compounds, and salts thereof (including pharmaceutically acceptable salts thereof). These cationic lipids are suitable for forming nucleic acid-lipid particles.

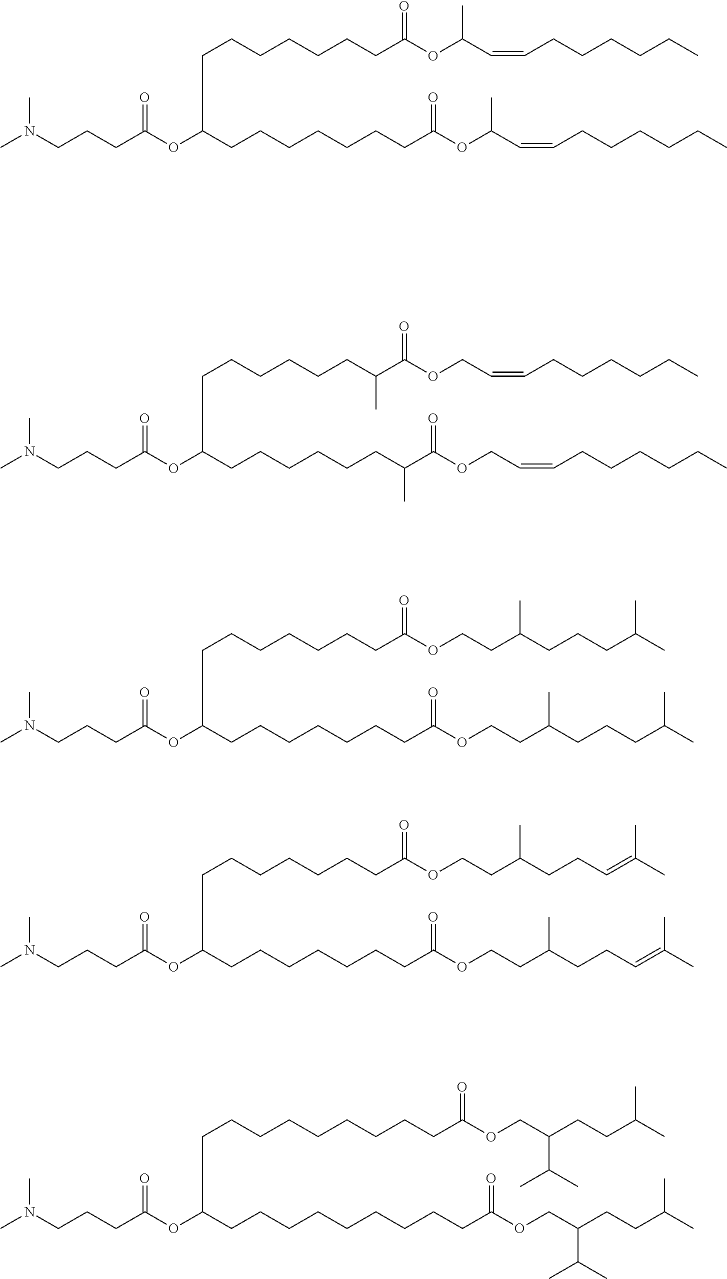

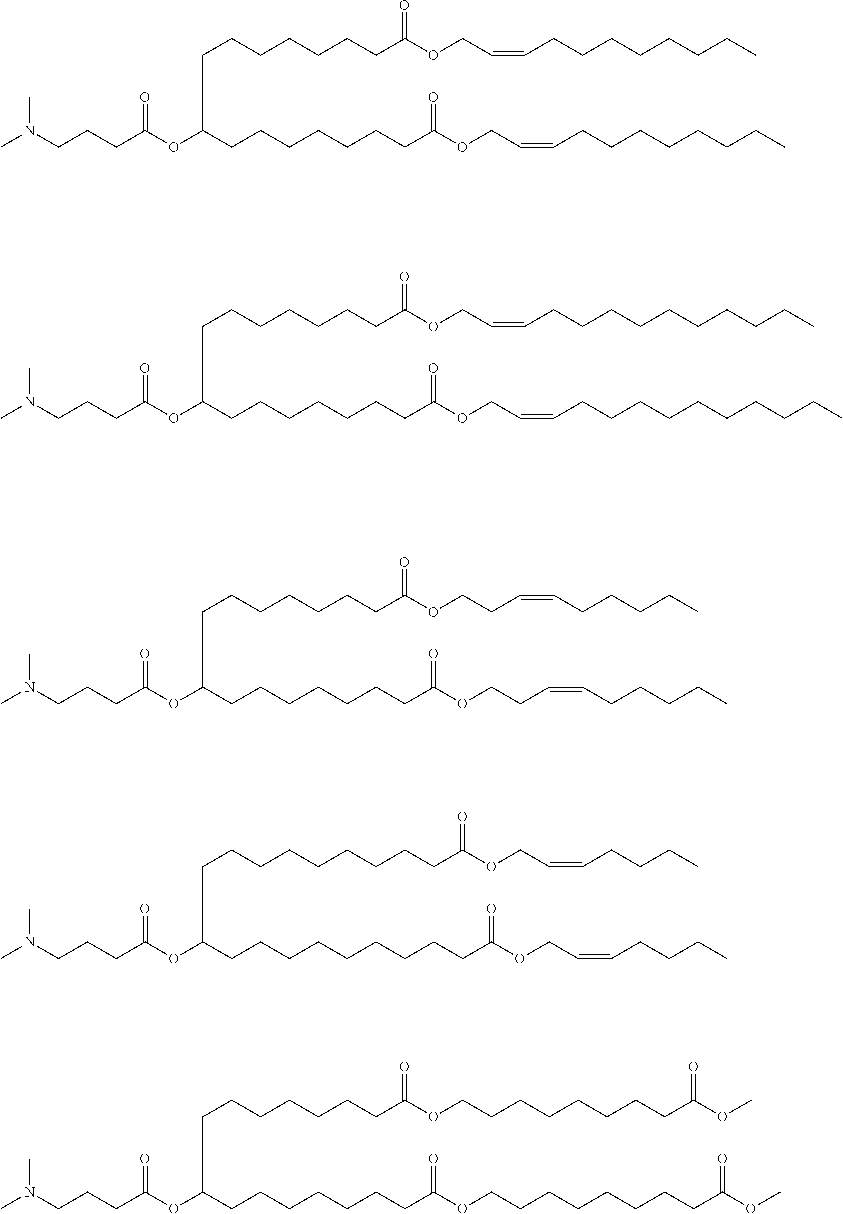

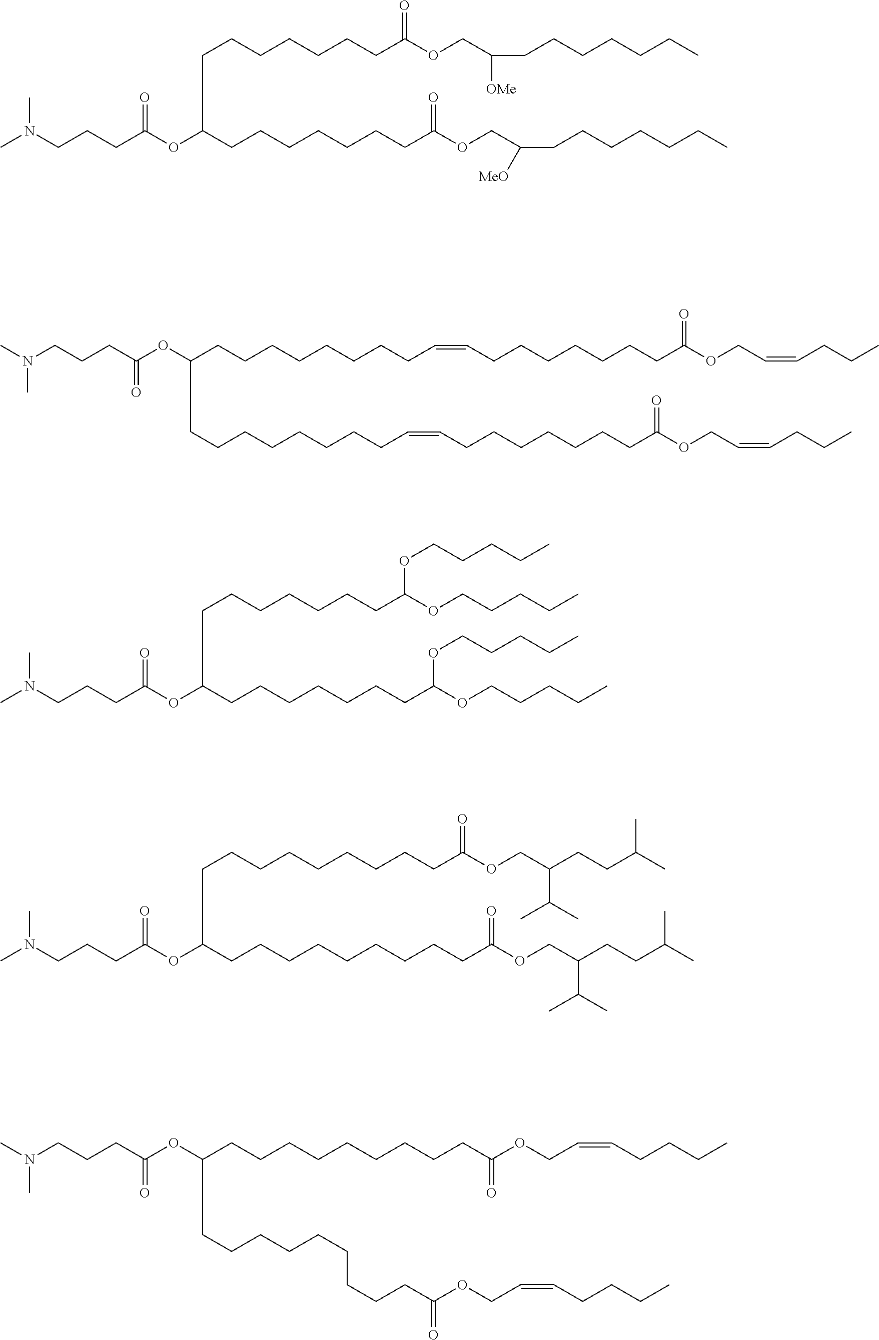

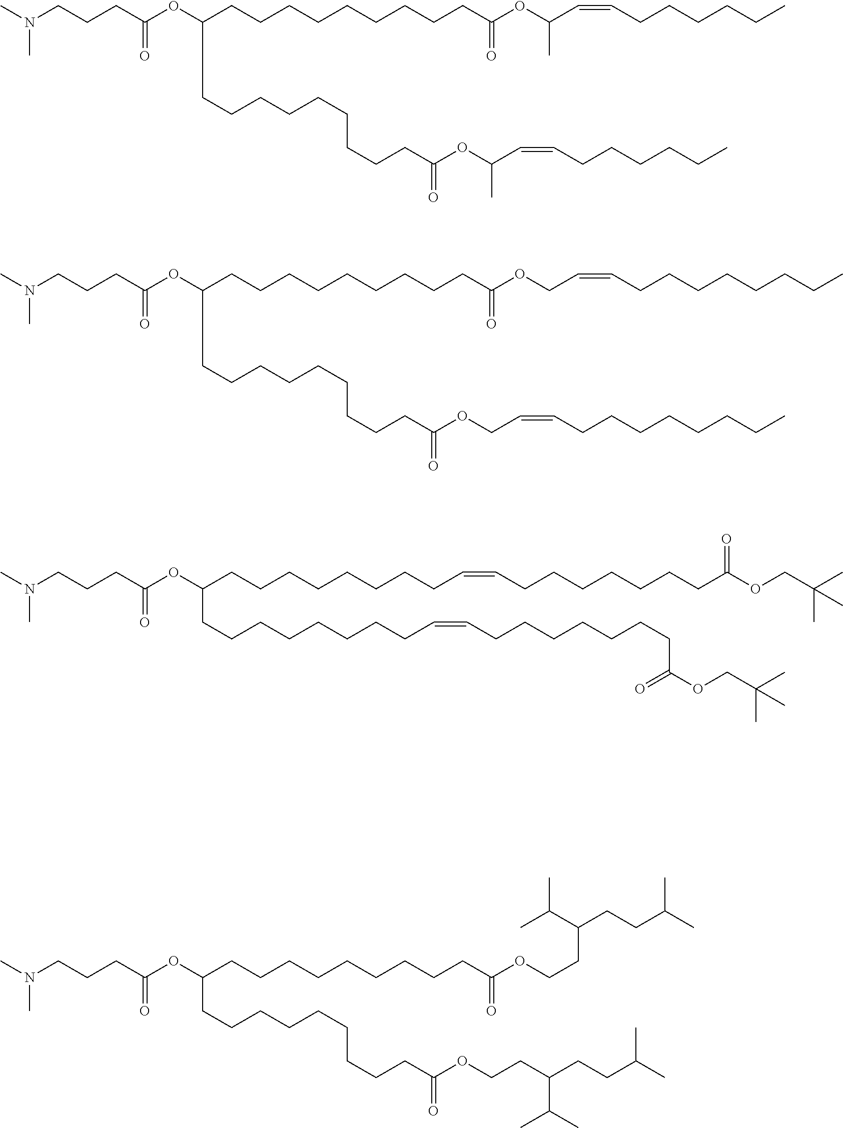

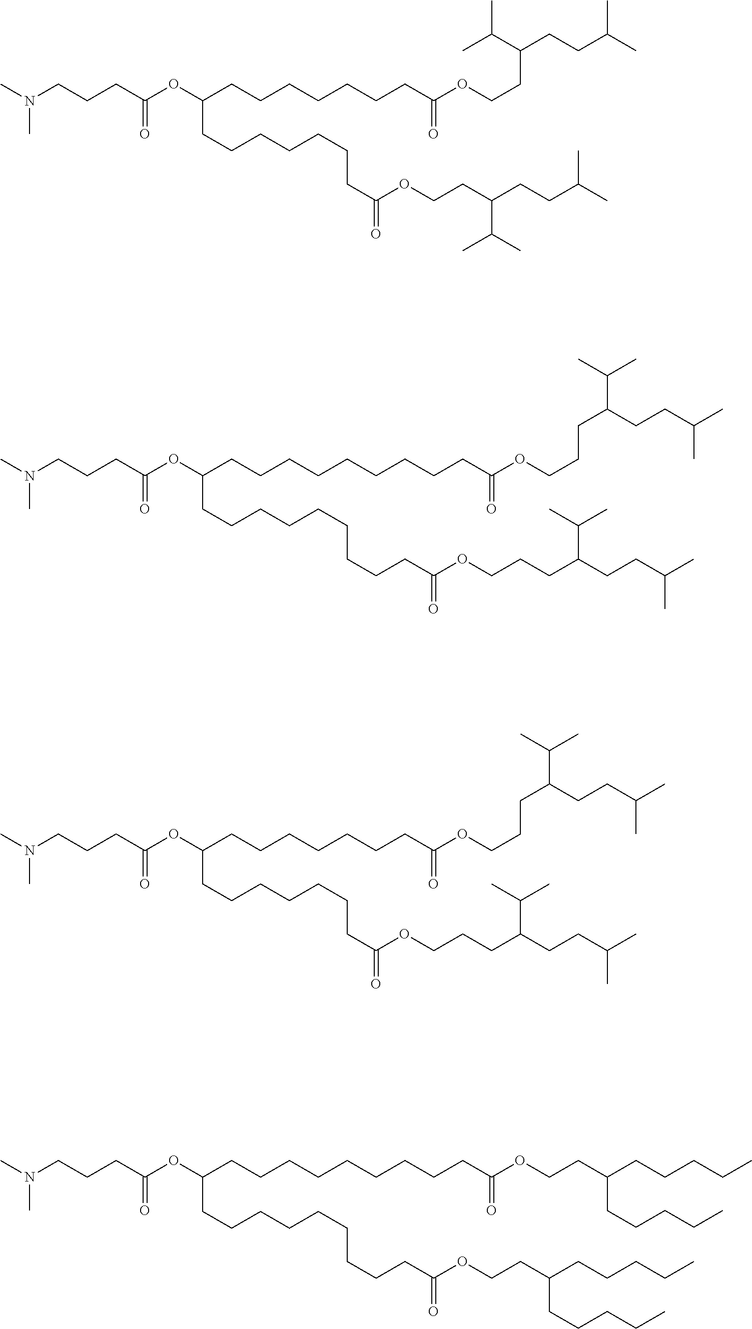

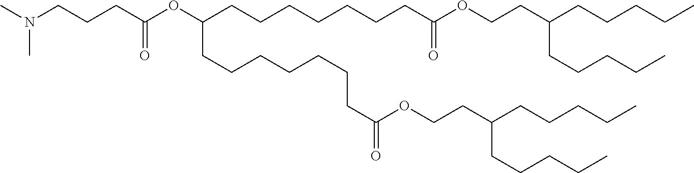 [SMILES: CCCCCC(CCCCC)CCOC(=O)CCCCCCCC(CCCCCCCC(=O)OCCC(CCCCC)CCCCC)OC(=O)CCCN(C)C]

In some embodiments, the cationic lipid of the present disclosure is selected from the following compounds, and salts thereof (including pharmaceutically acceptable salts thereof):

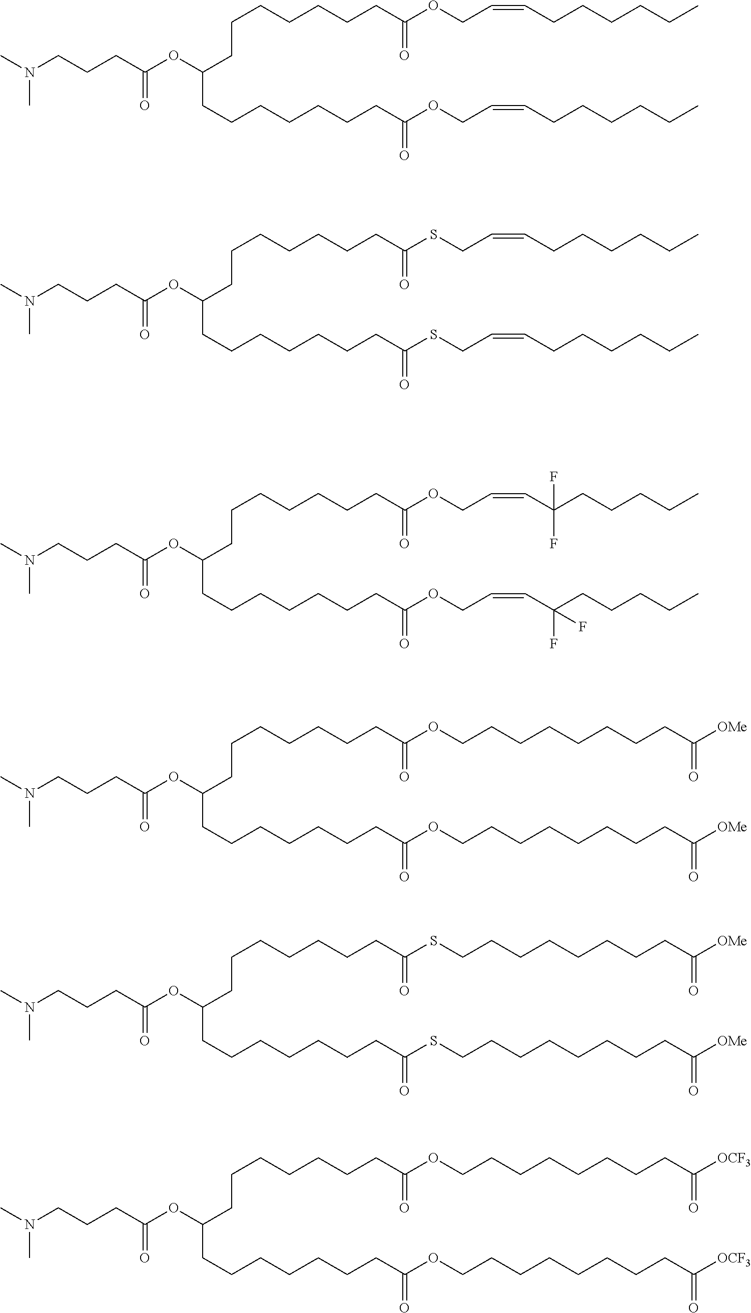

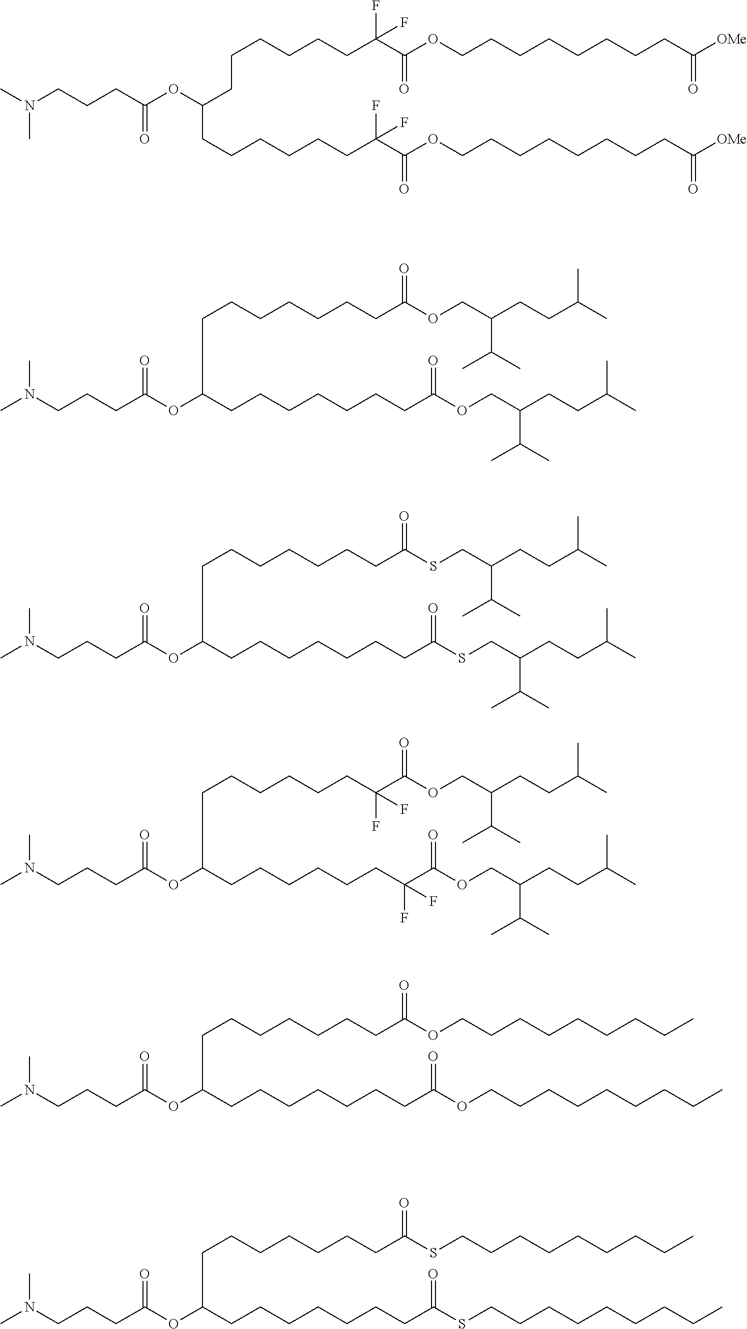

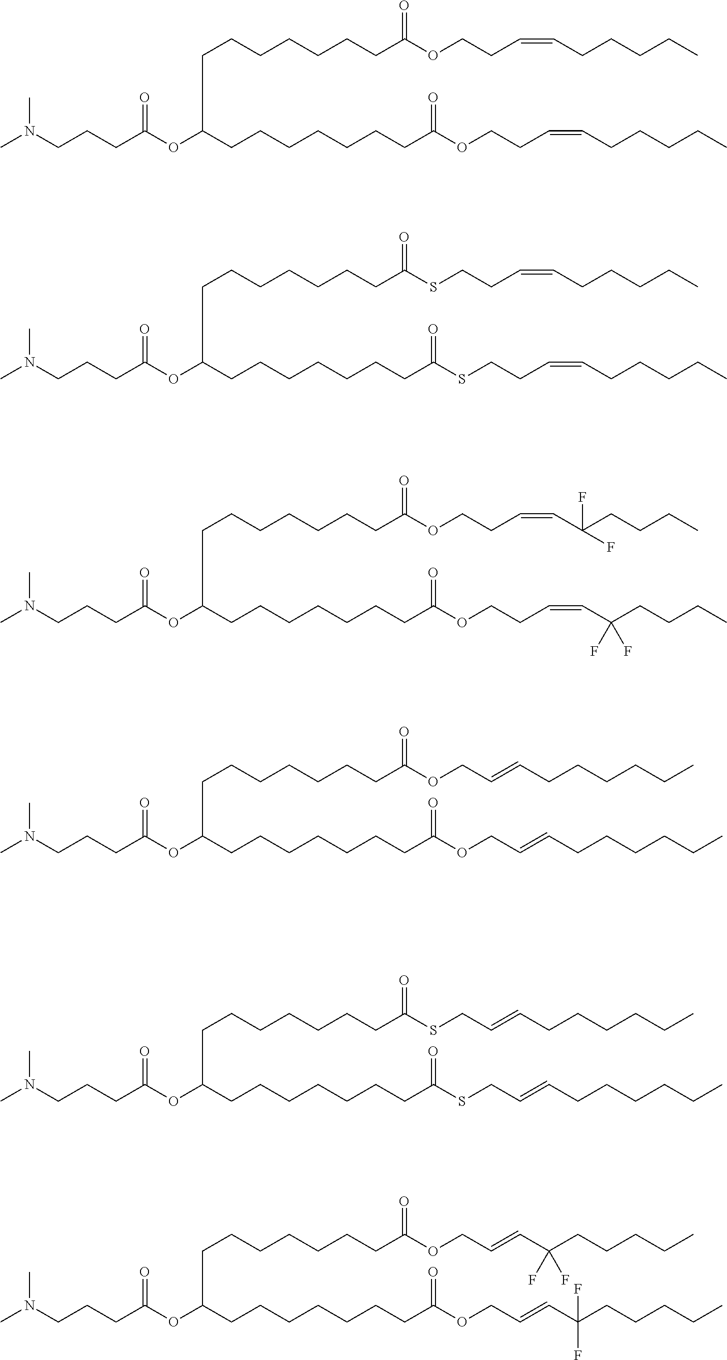

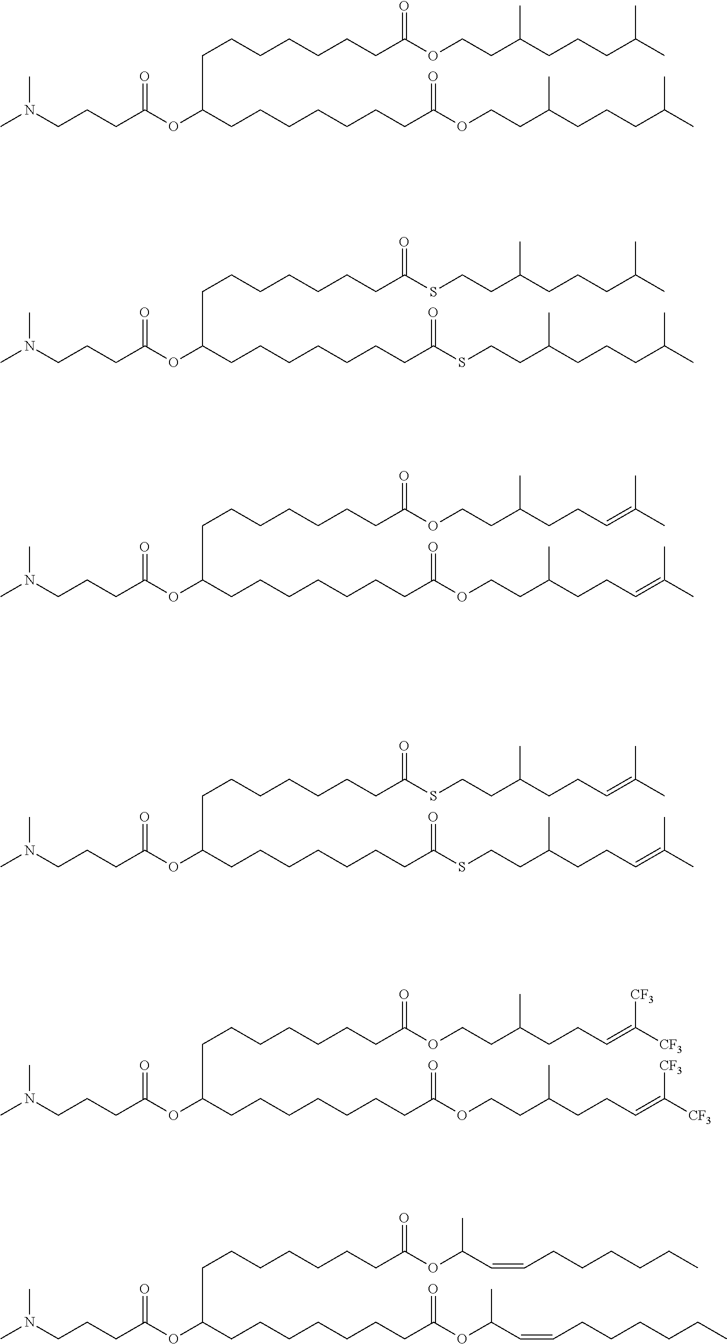

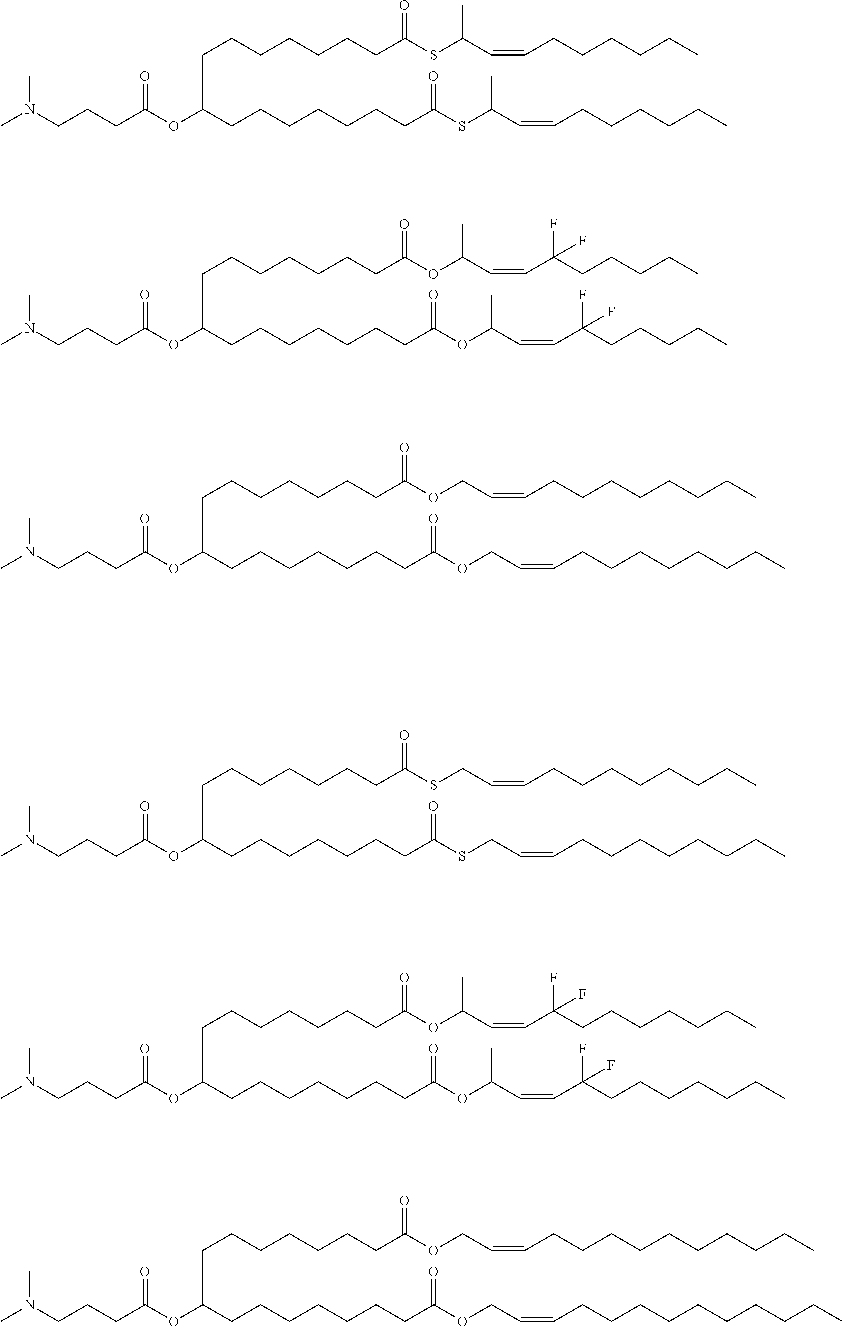

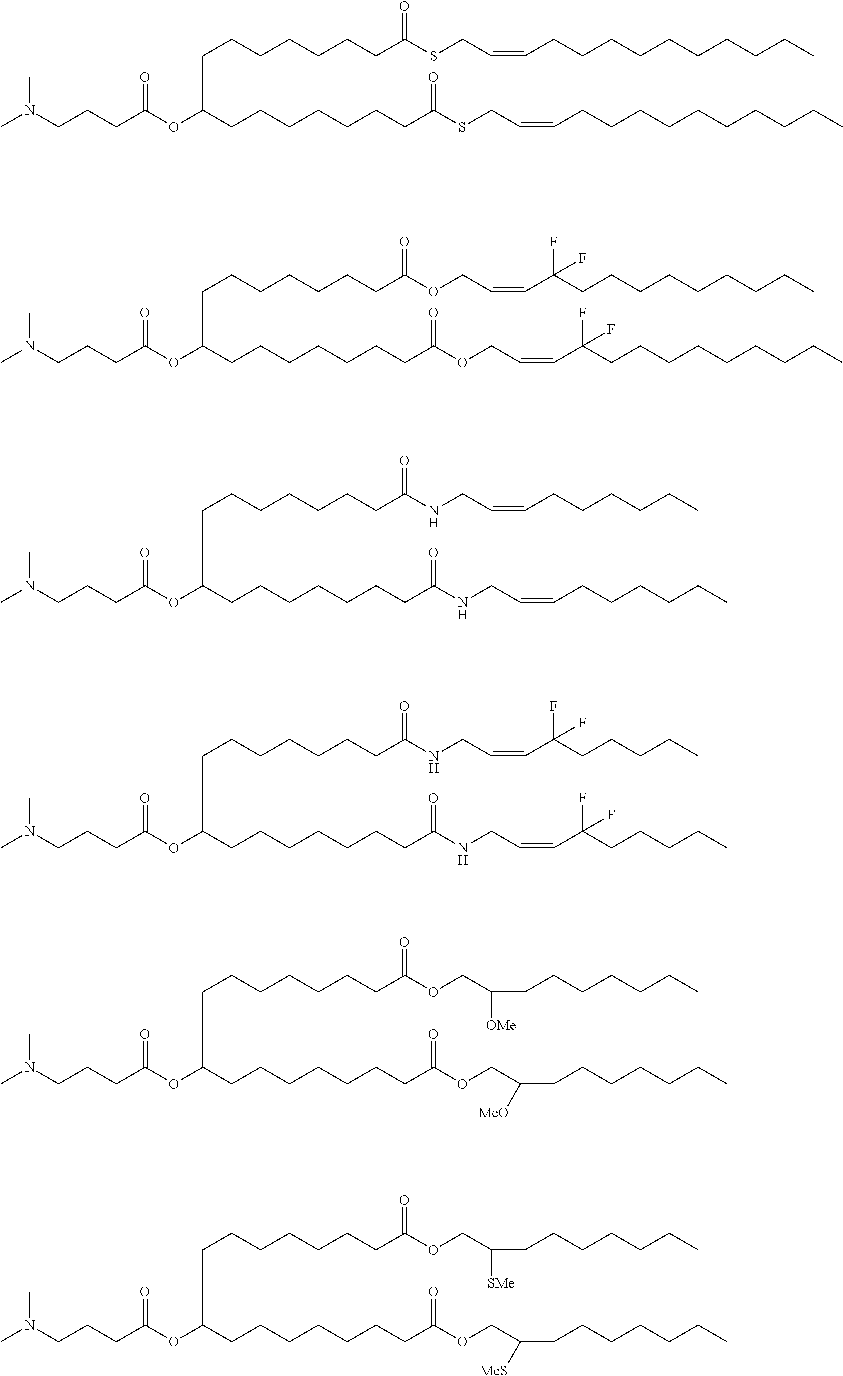

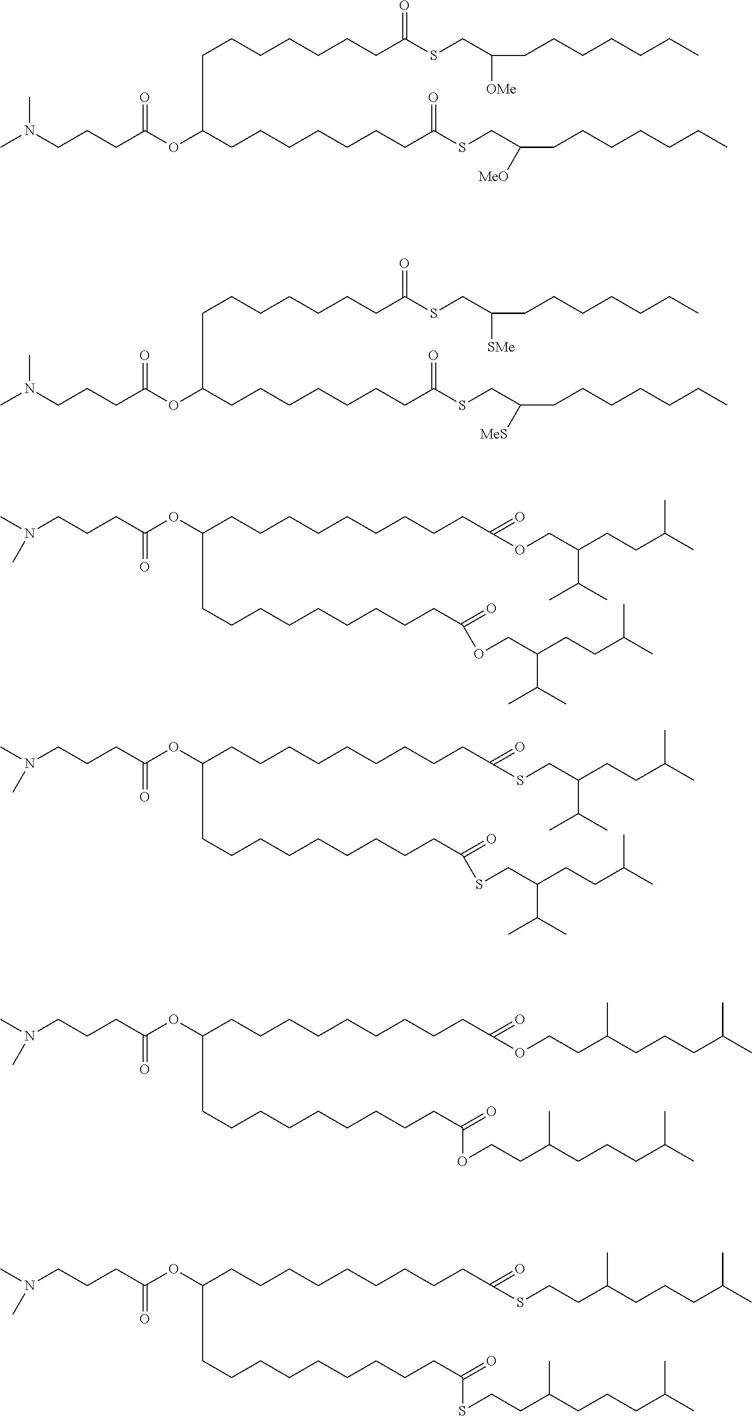

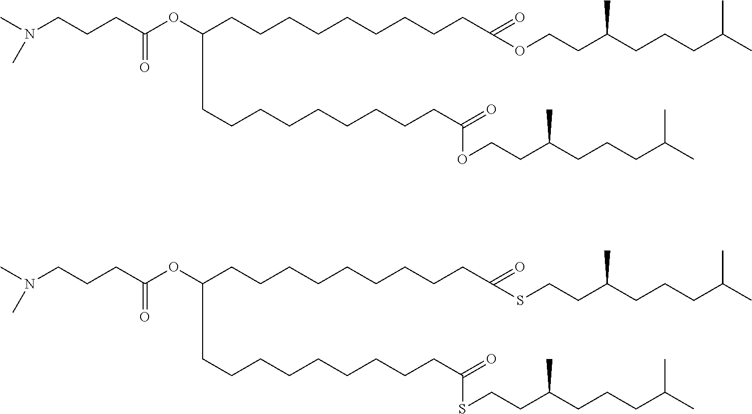

In some embodiments, the cationic lipid is selected from the following compounds, and salts thereof (including pharmaceutically acceptable salts thereof):

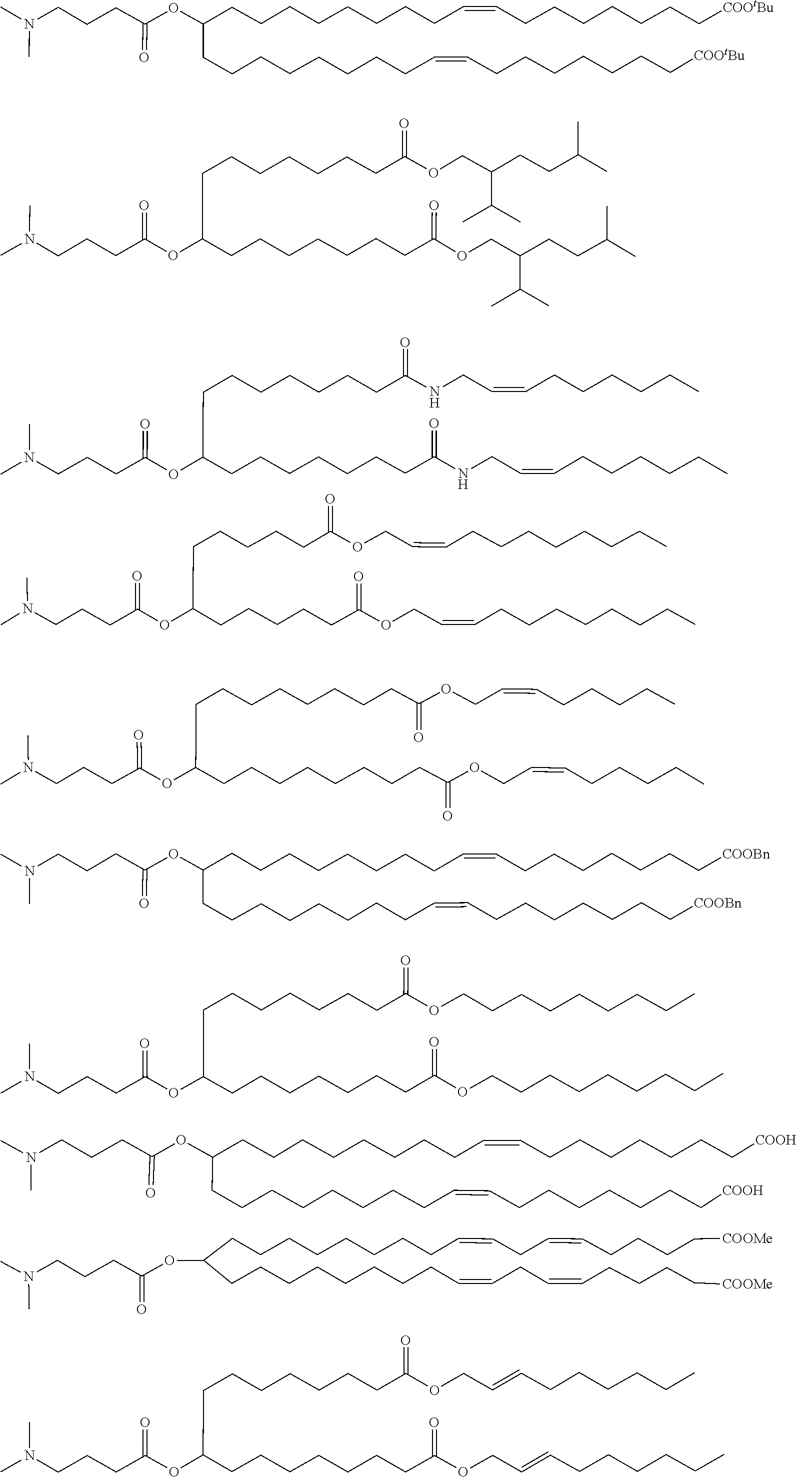

Additional representative cationic lipids include, but are not limited to:

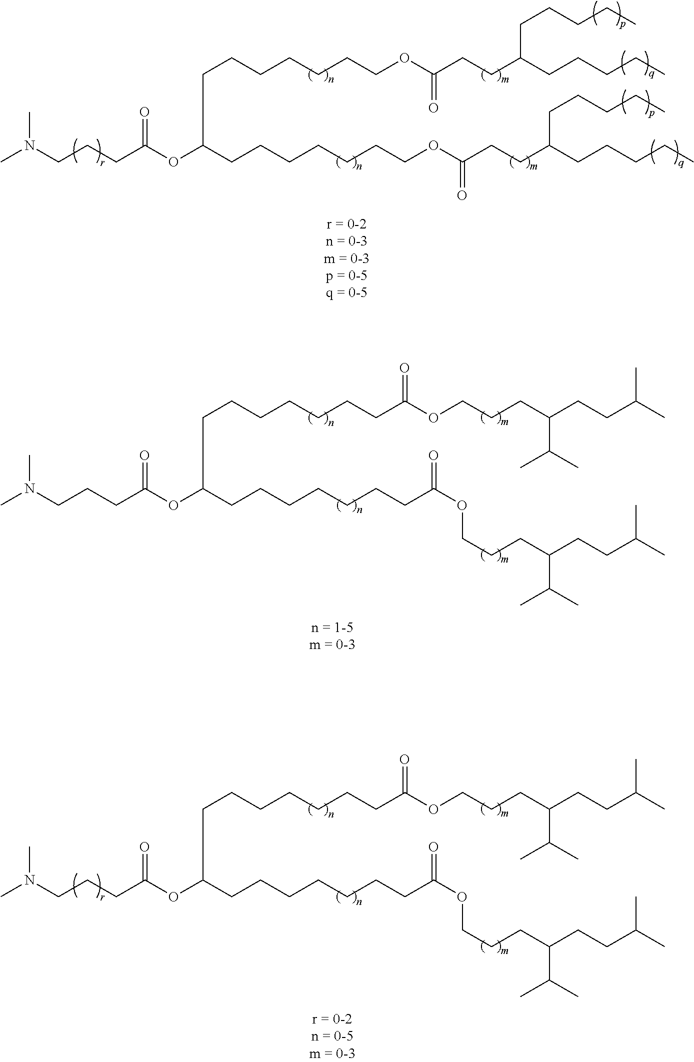

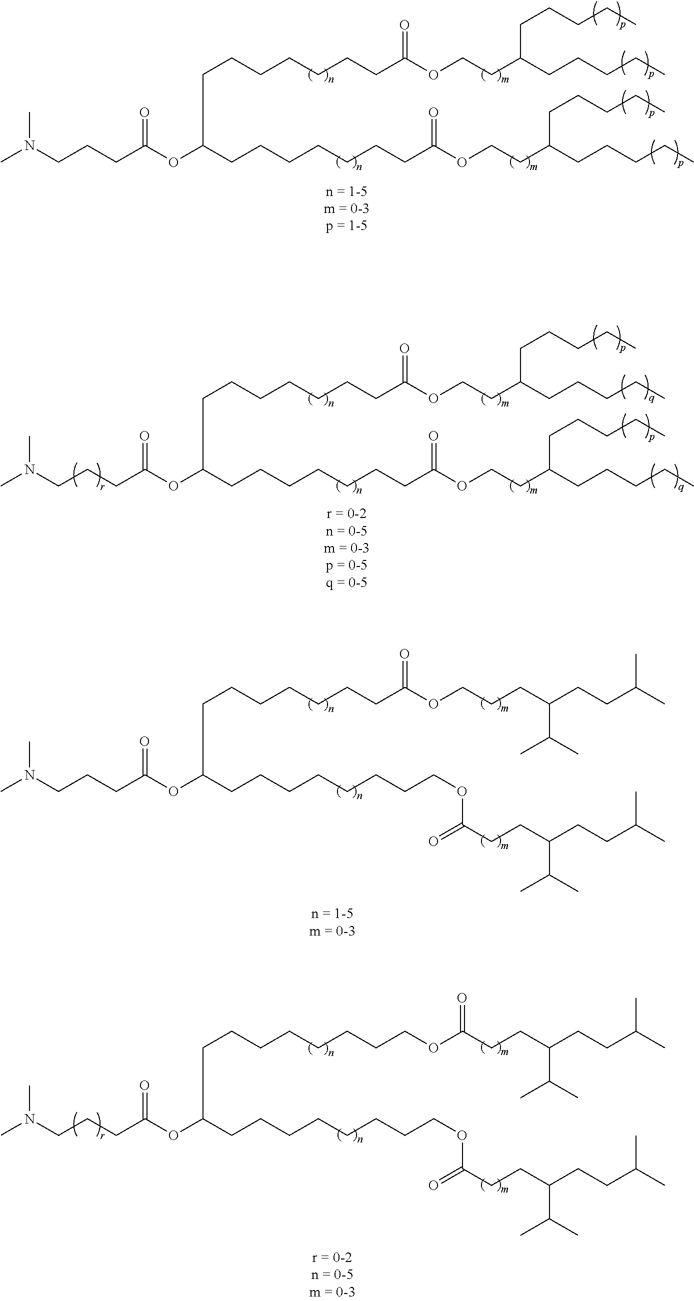

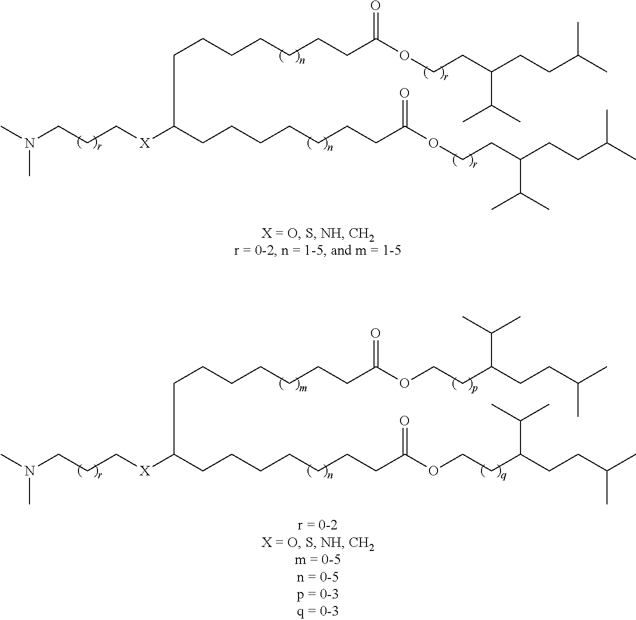

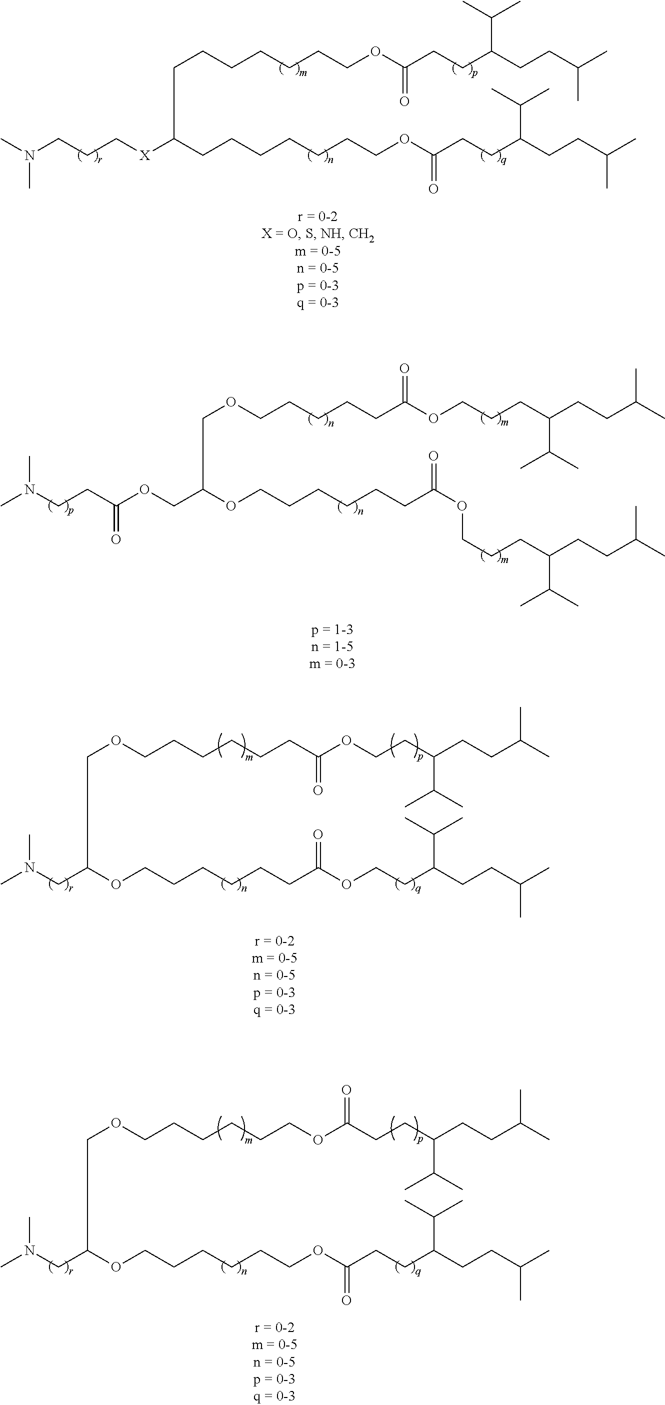

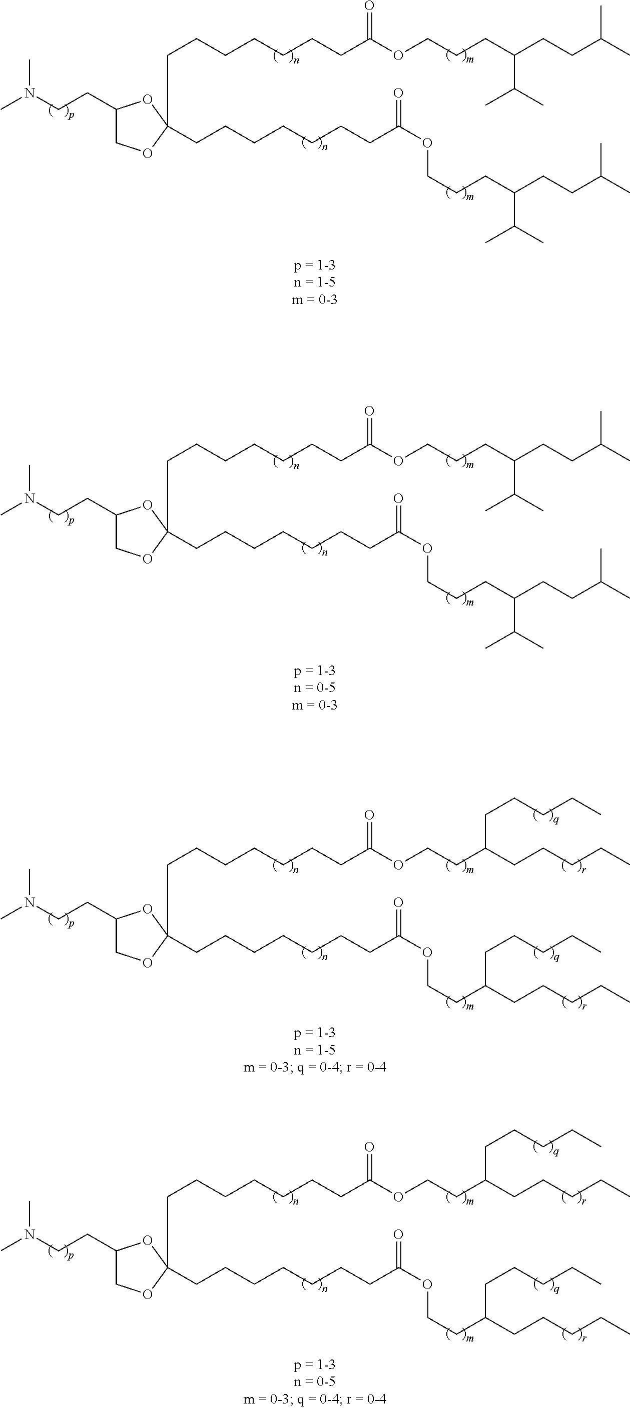

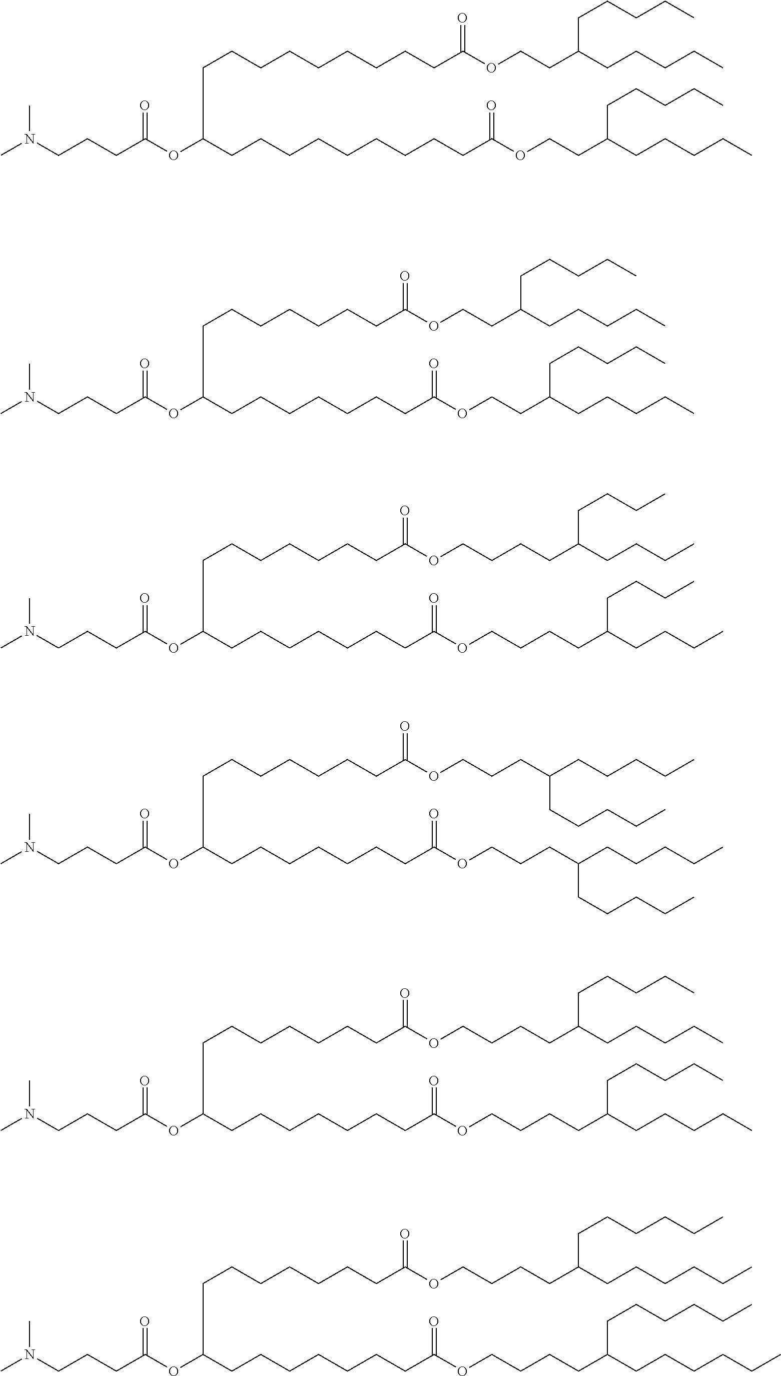

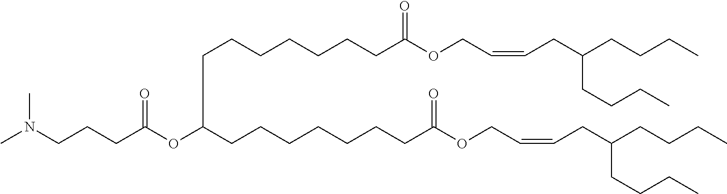 [SMILES: CCCCC(C/C=C\COC(=O)CCCCCCCC(CCCCCCCC(=O)OC/C=C\CC(CCCC)CCCC)OC(=O)CCCN(C)C)CCCC]

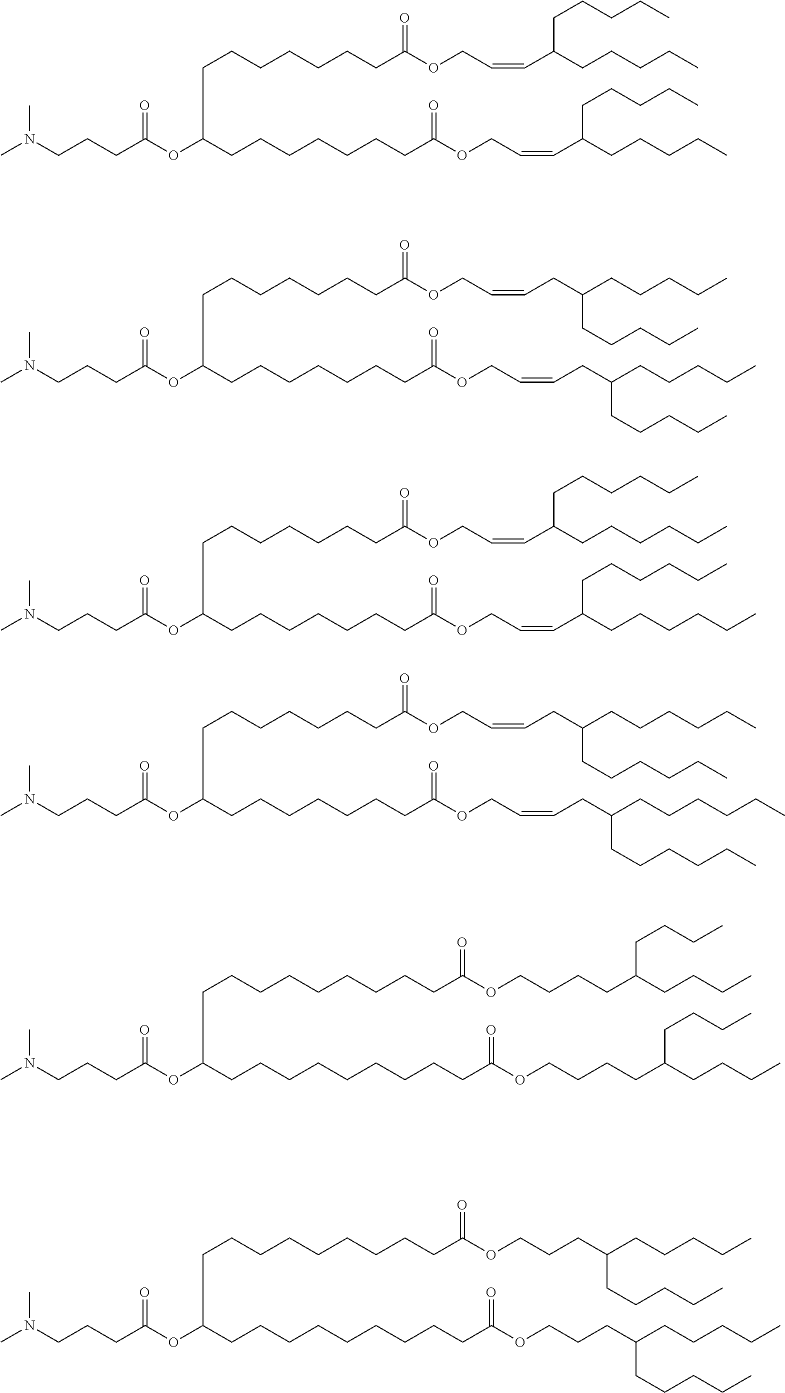

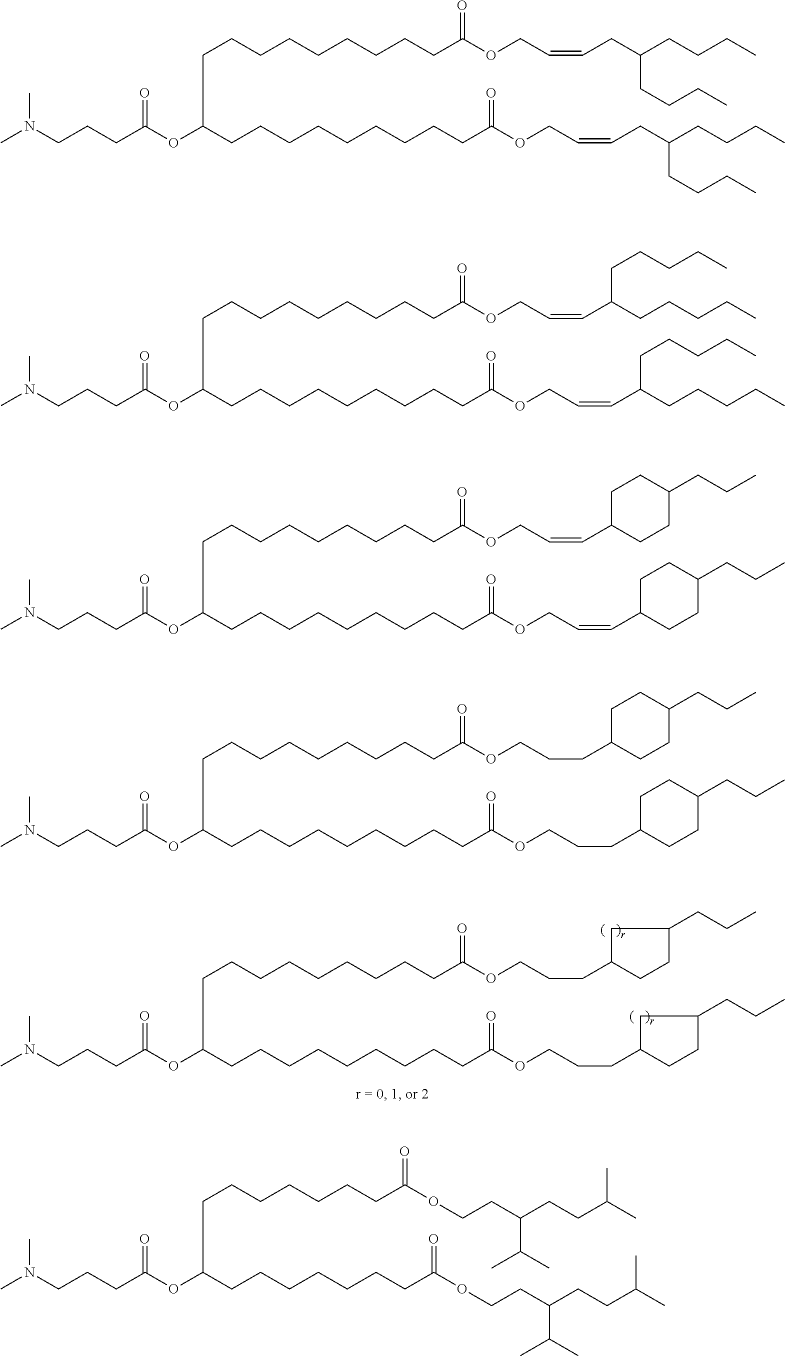

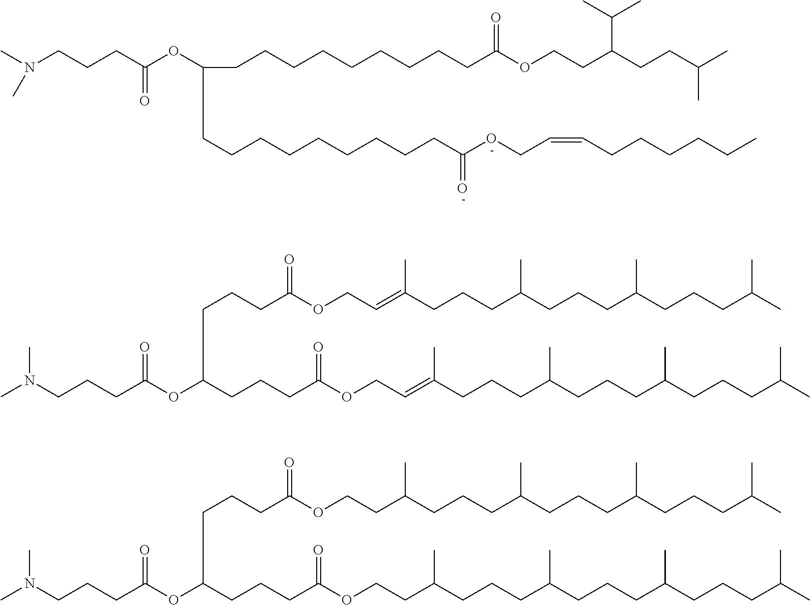

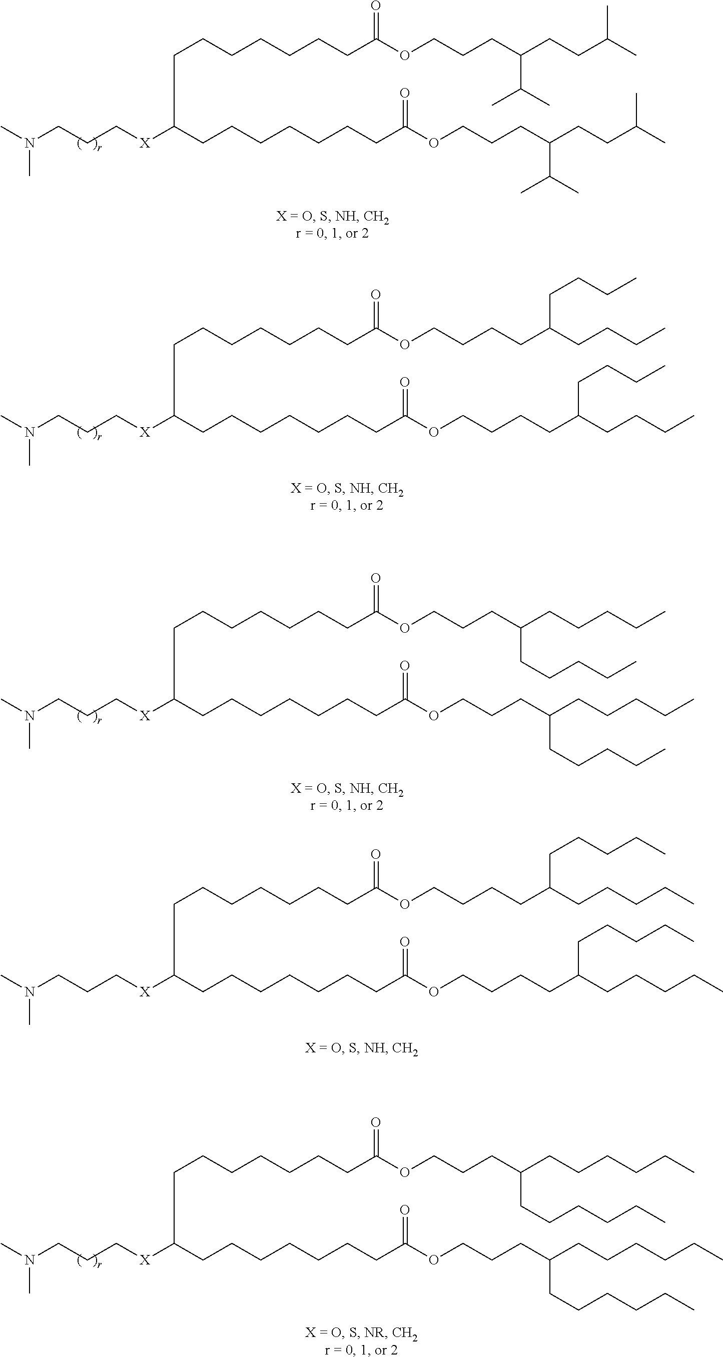

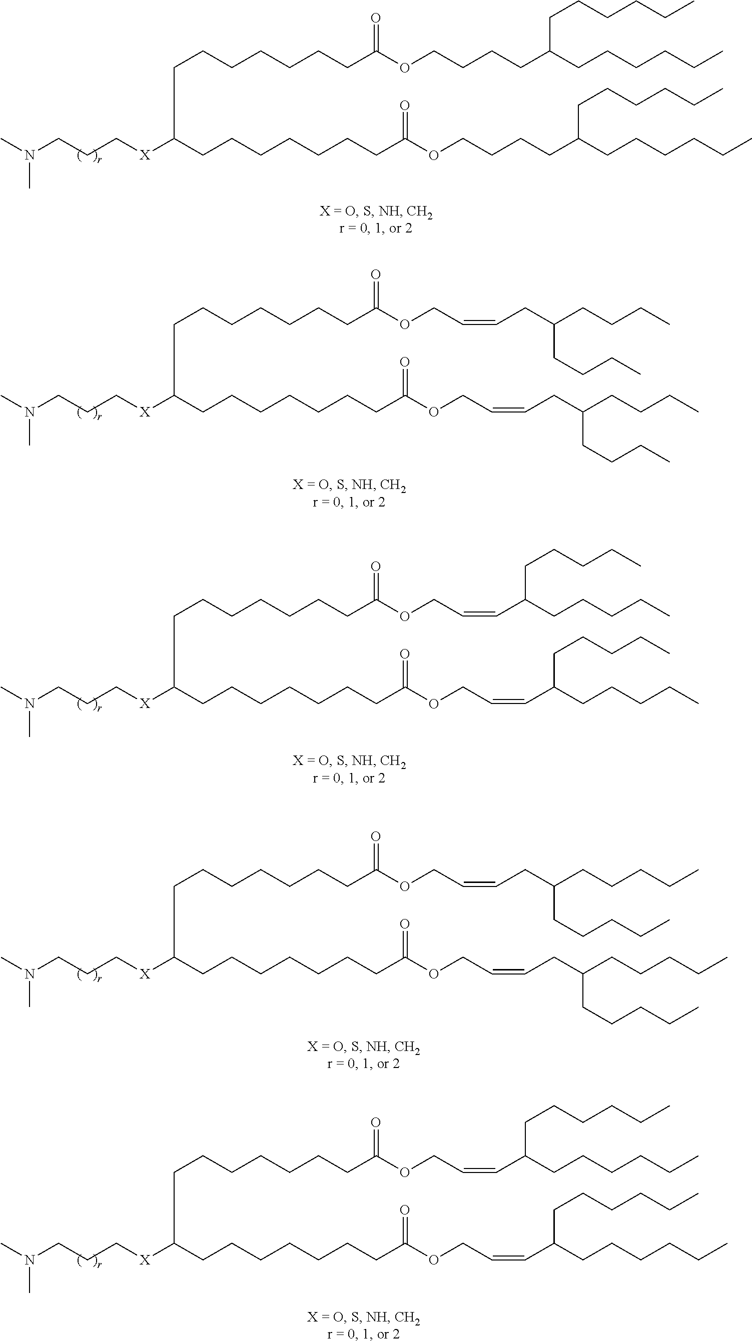

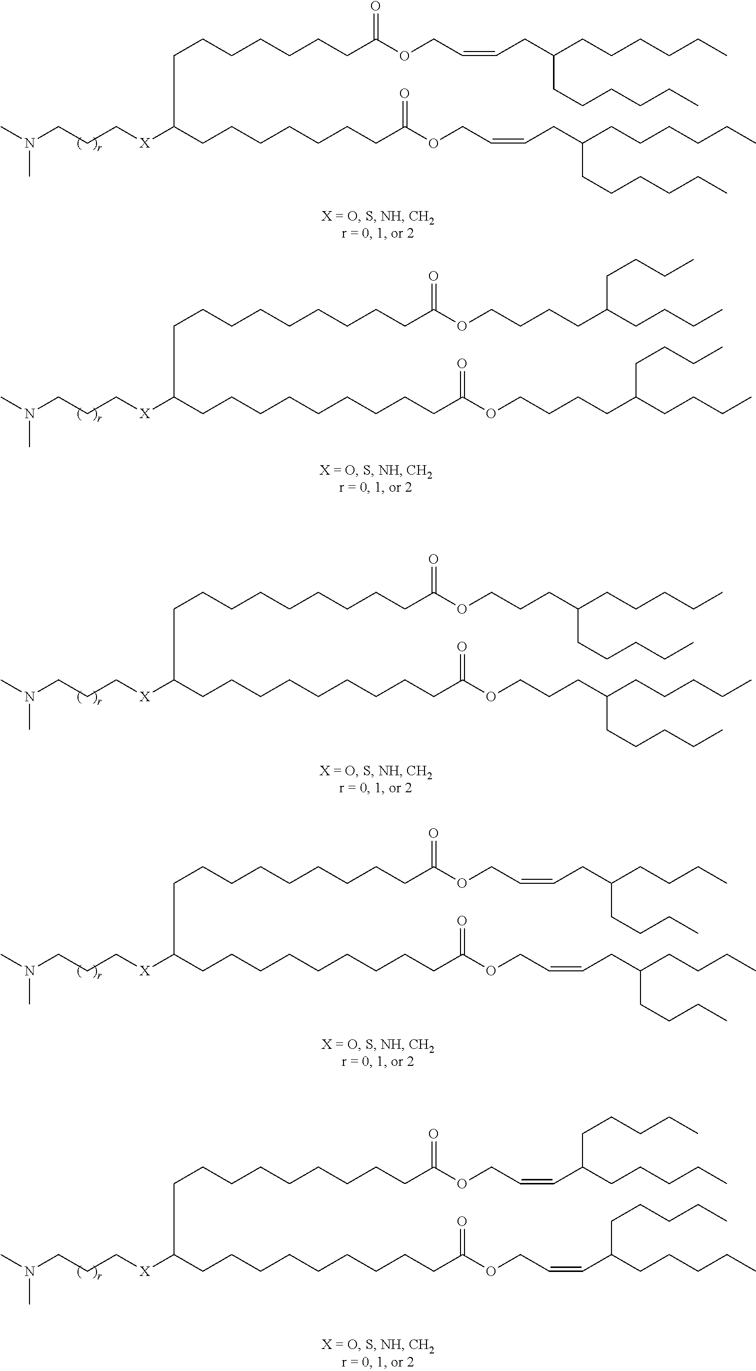

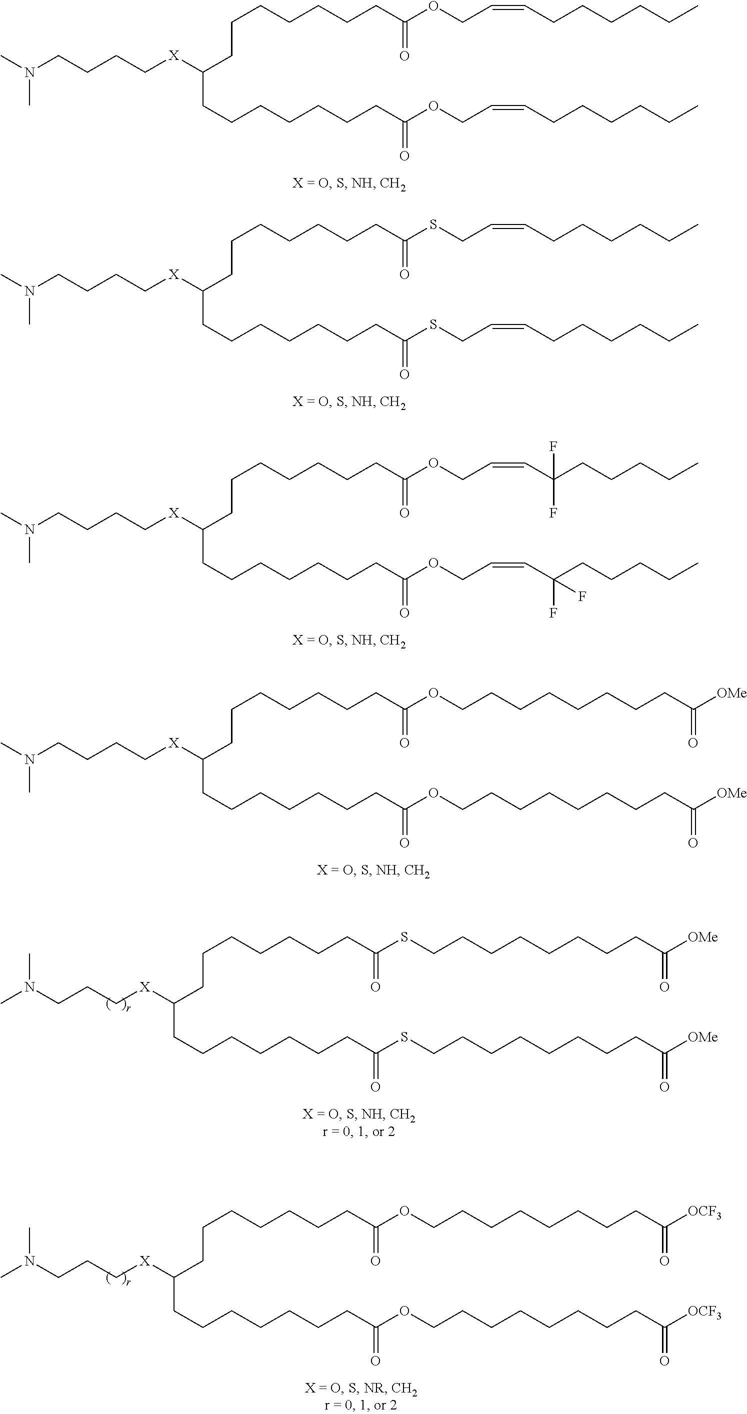

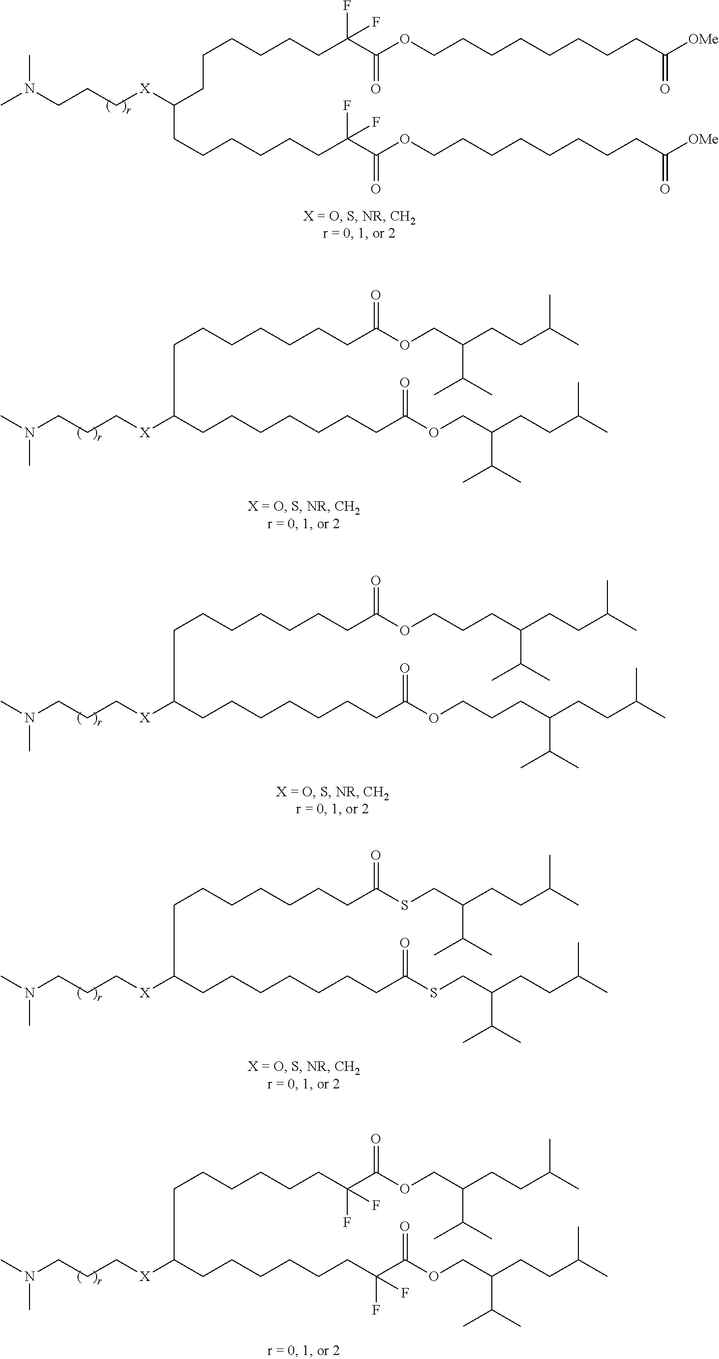

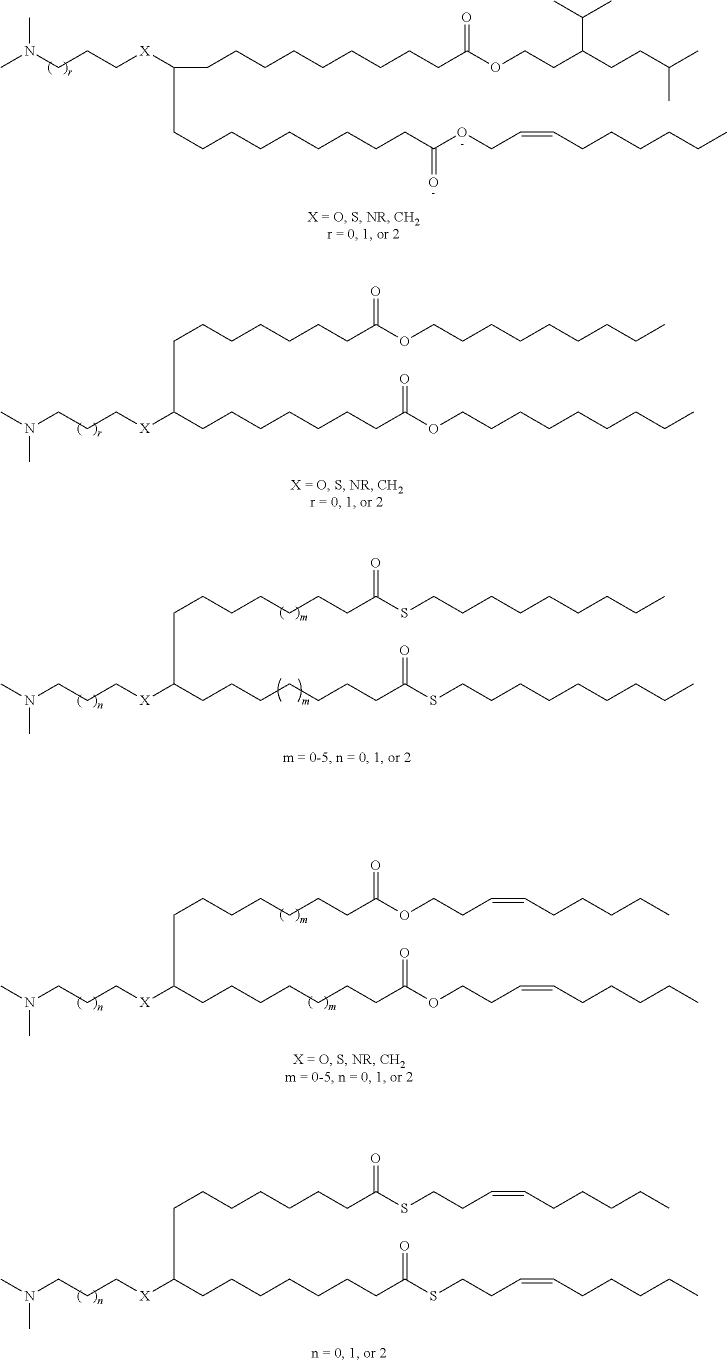

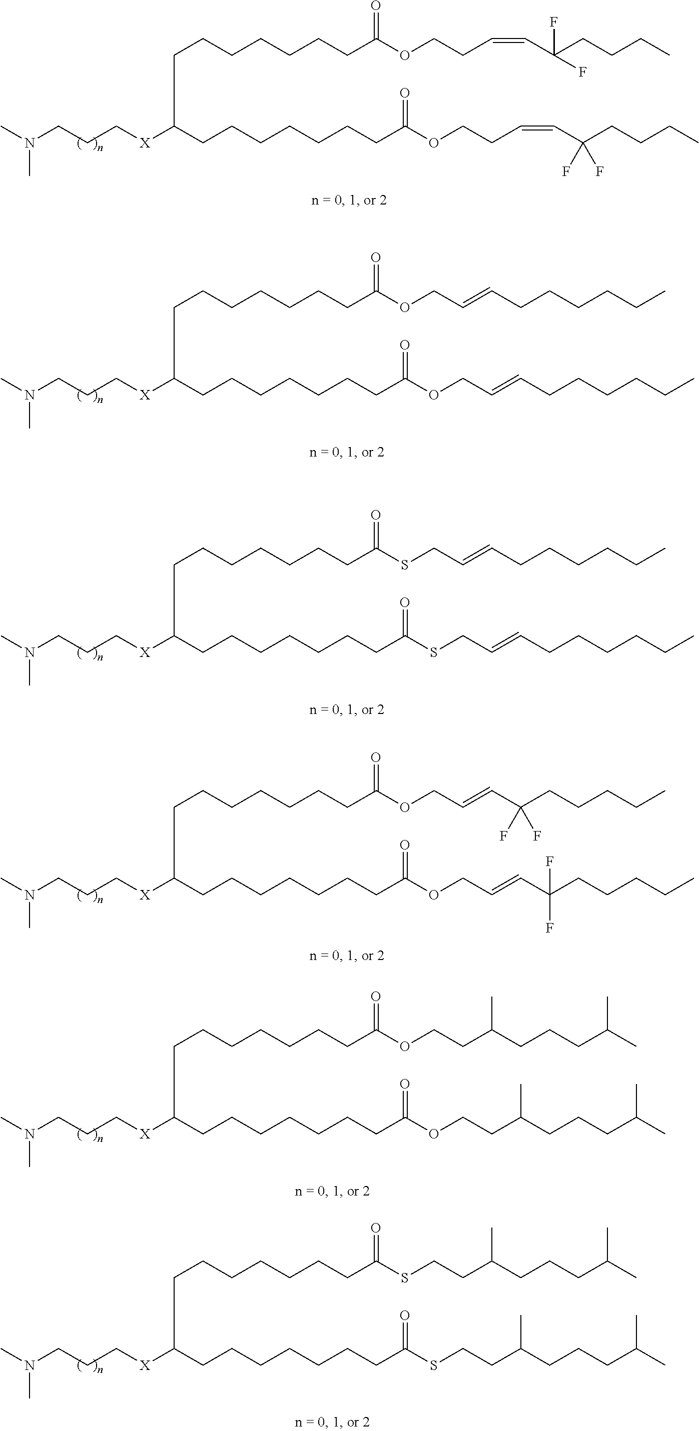

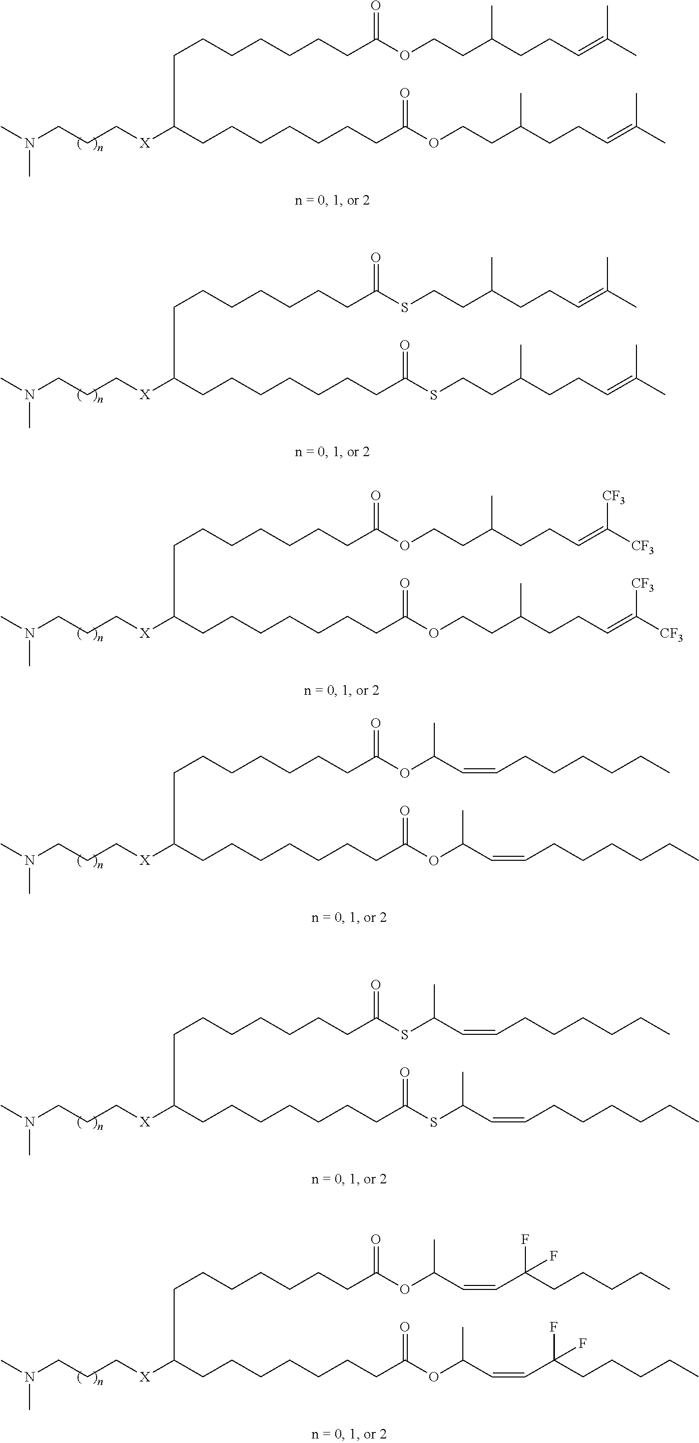

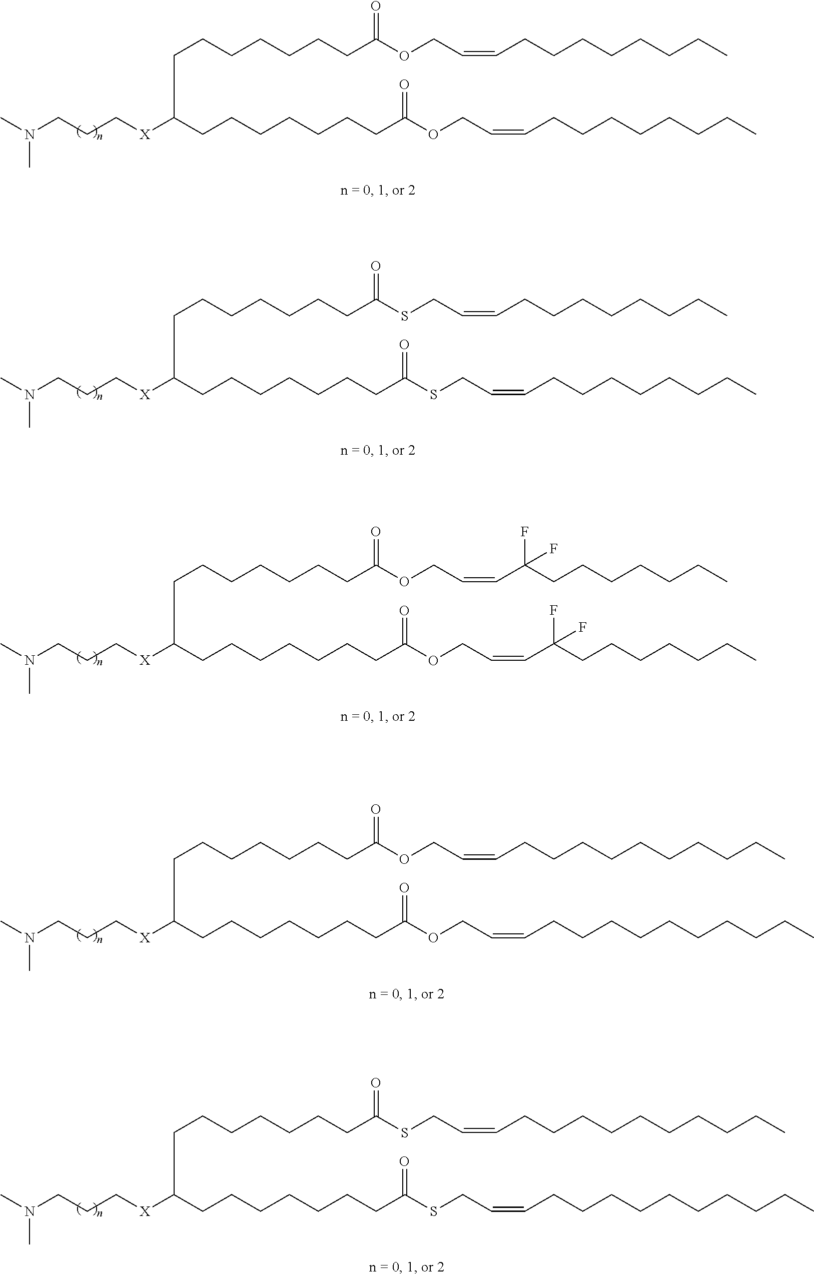

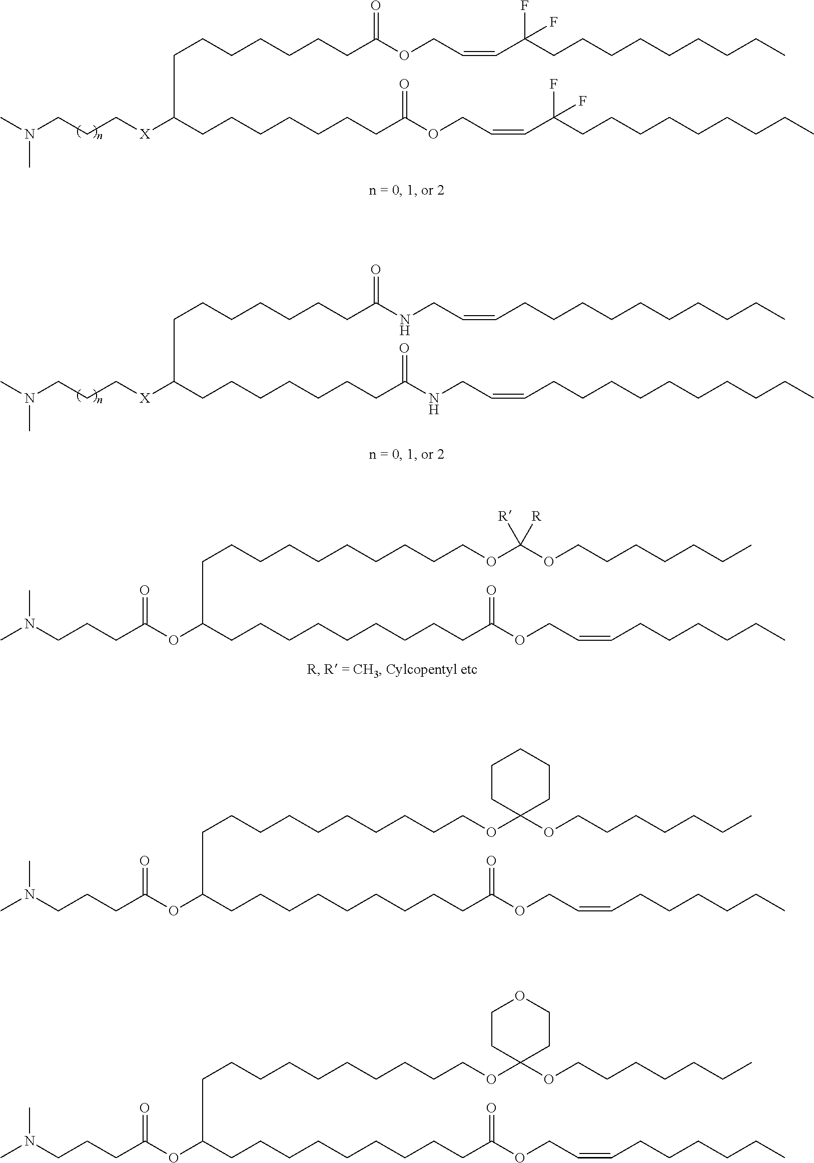

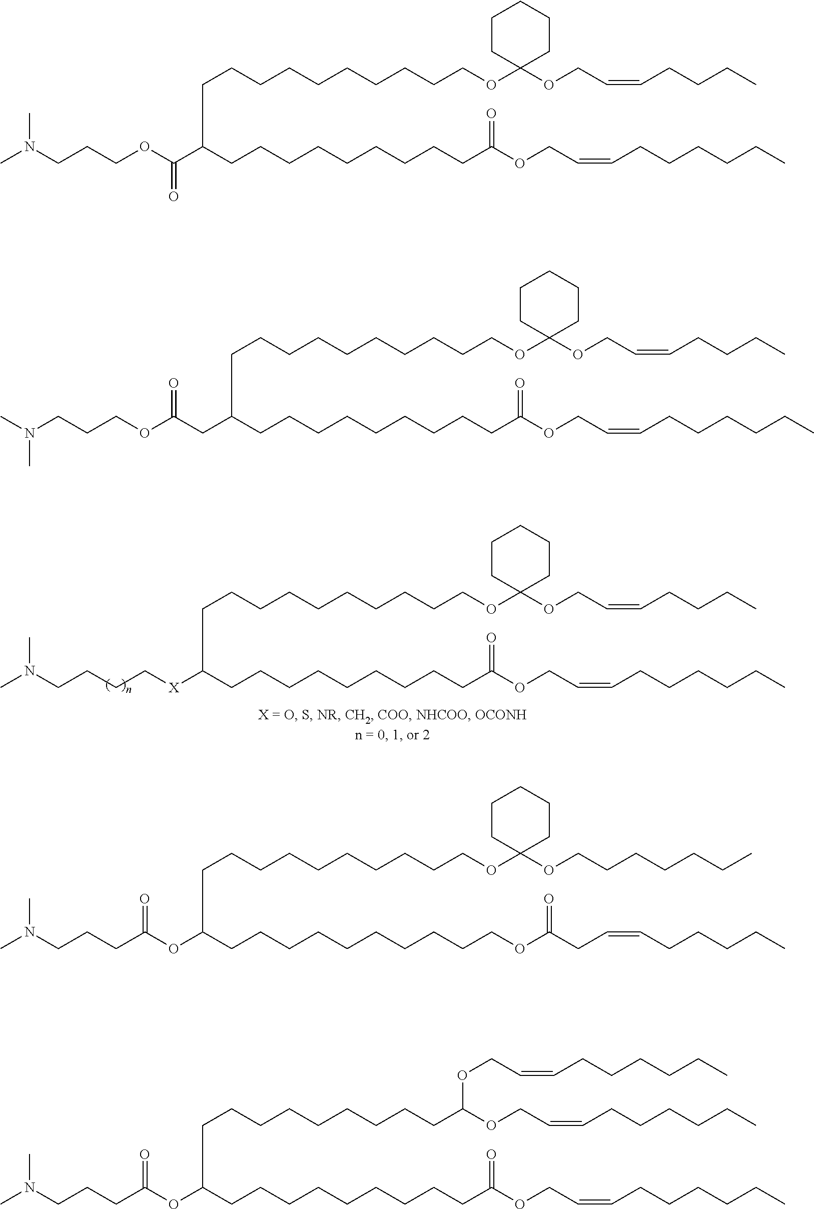

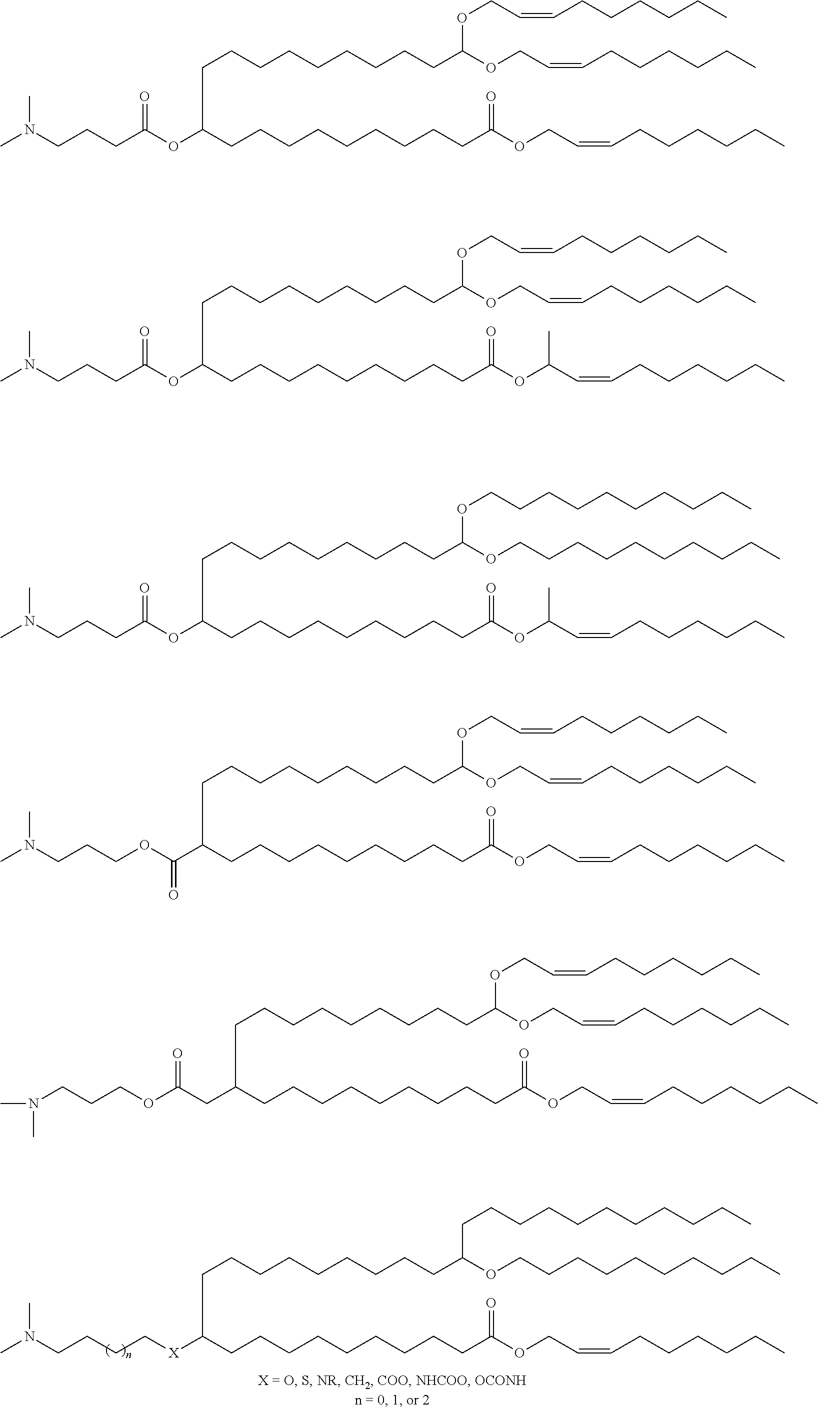

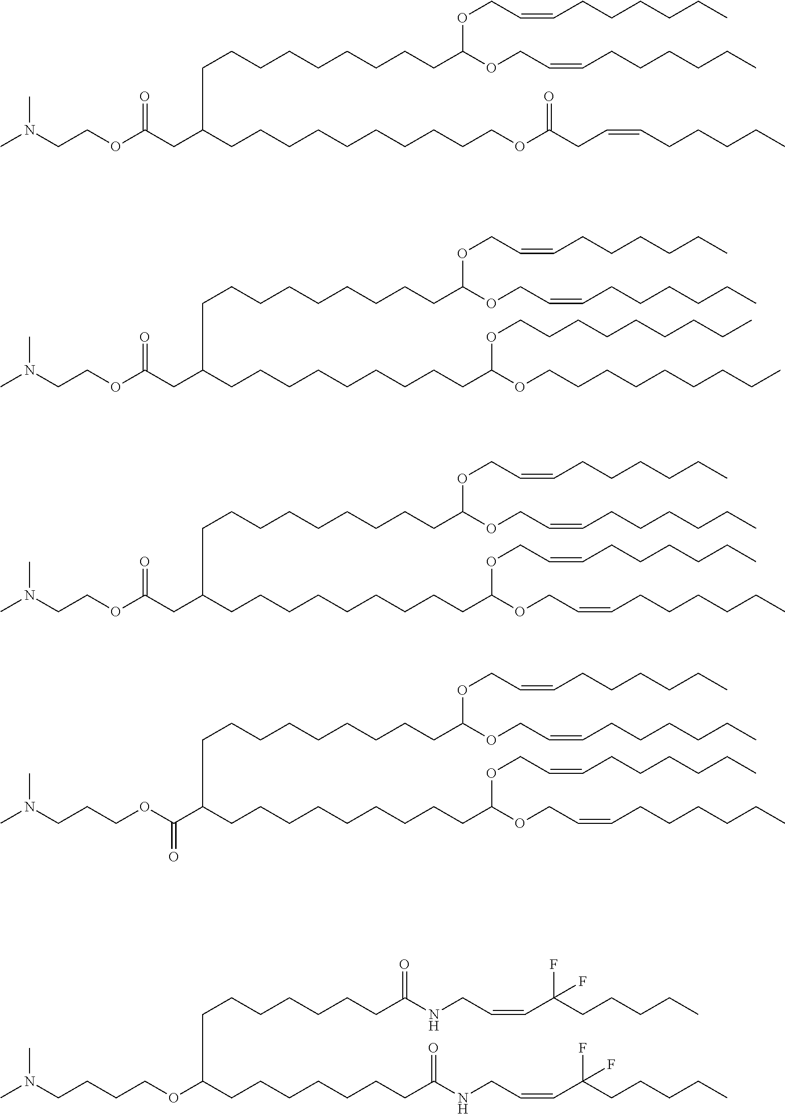

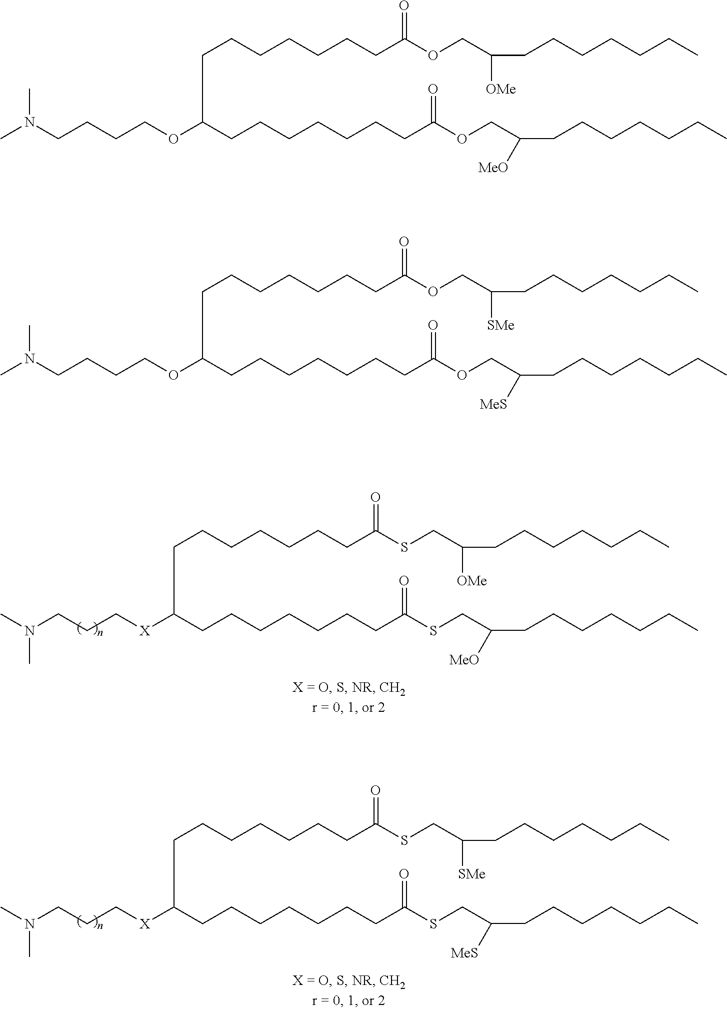

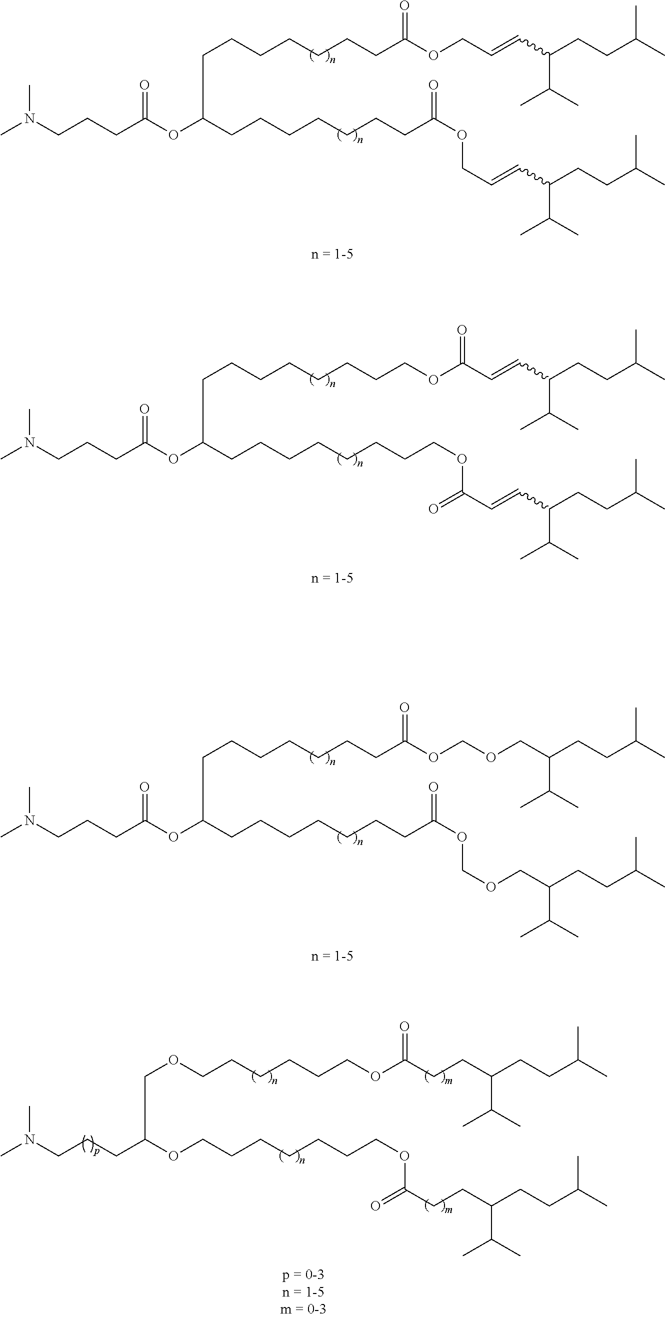

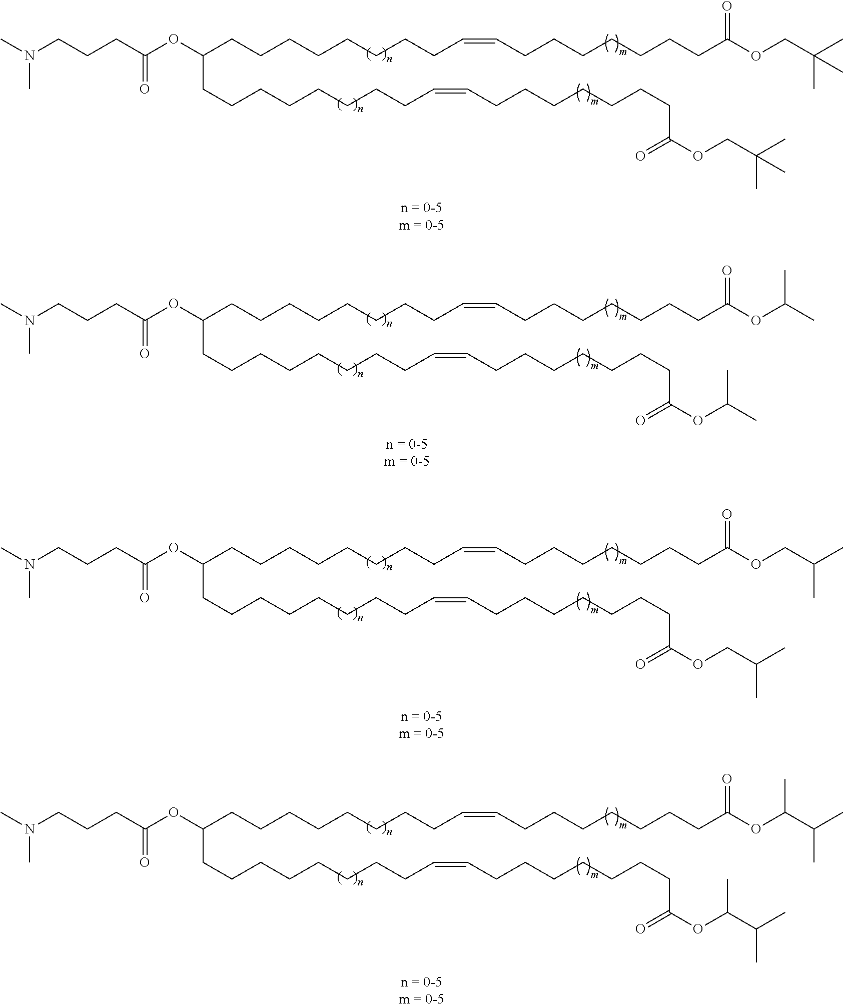

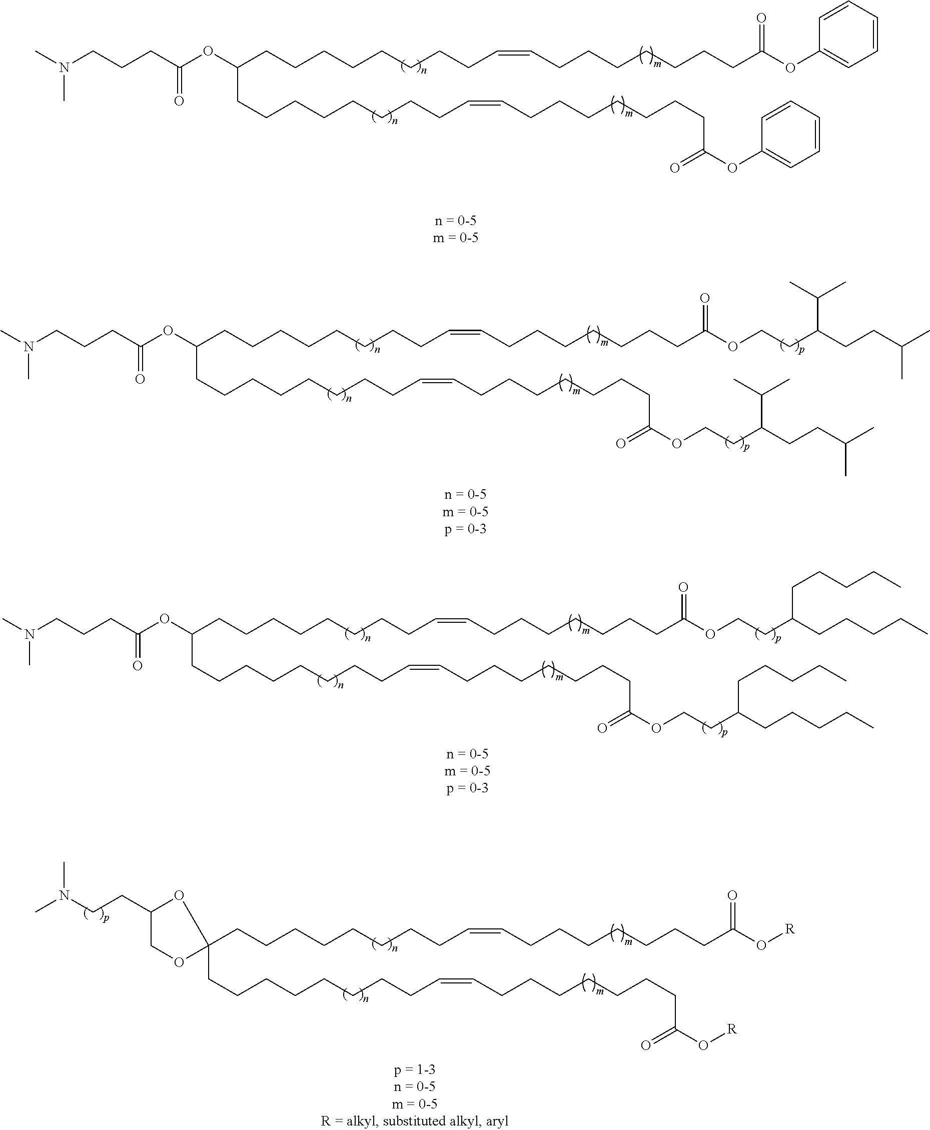

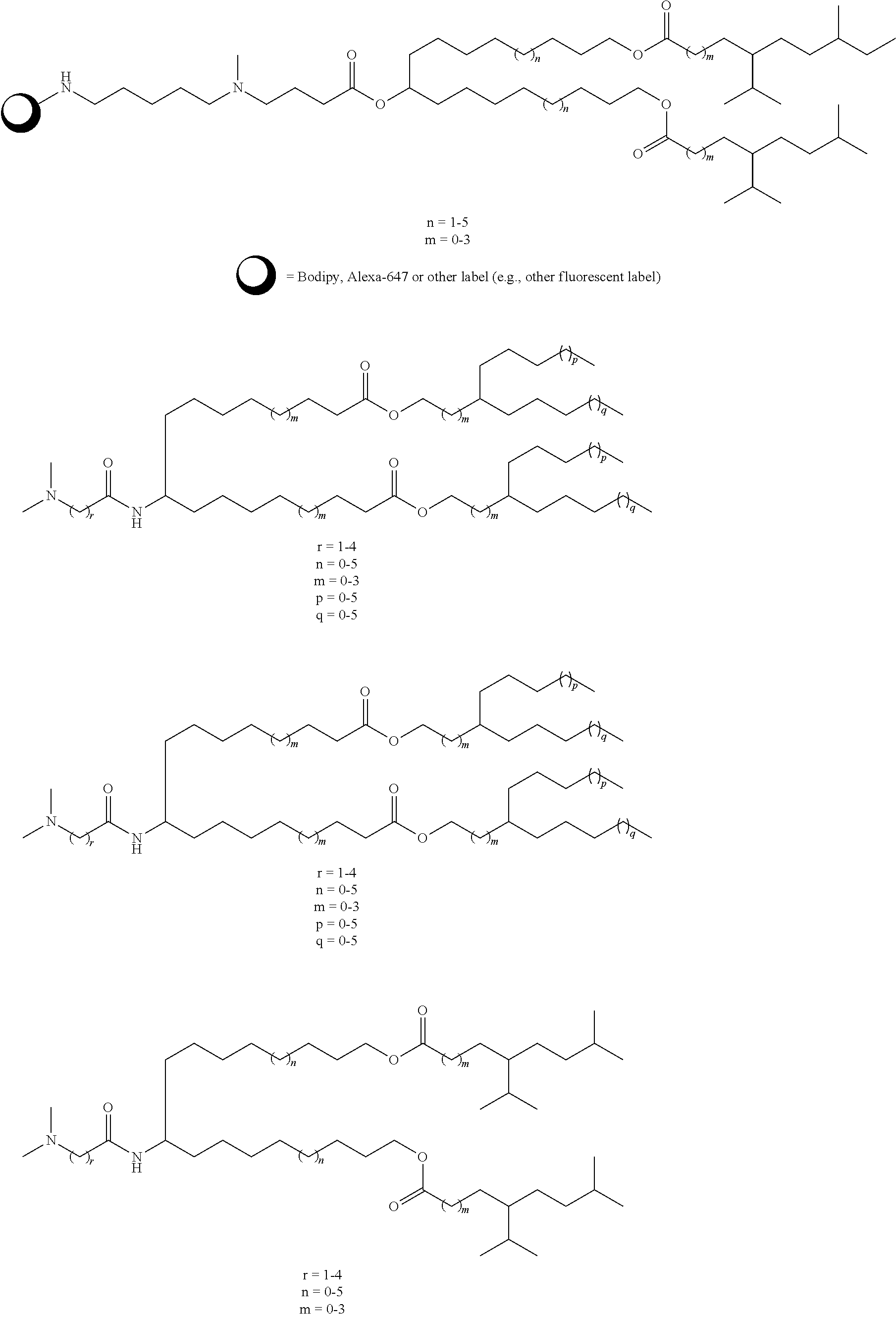

In some embodiments, a LNP described herein comprises a lipid, e.g., an ionizable lipid, disclosed in US 2015/0005363, which is incorporated herein by reference in its entirety.

In some embodiments, the lipids disclosed in US 2015/0005363 include, but are not limited to:

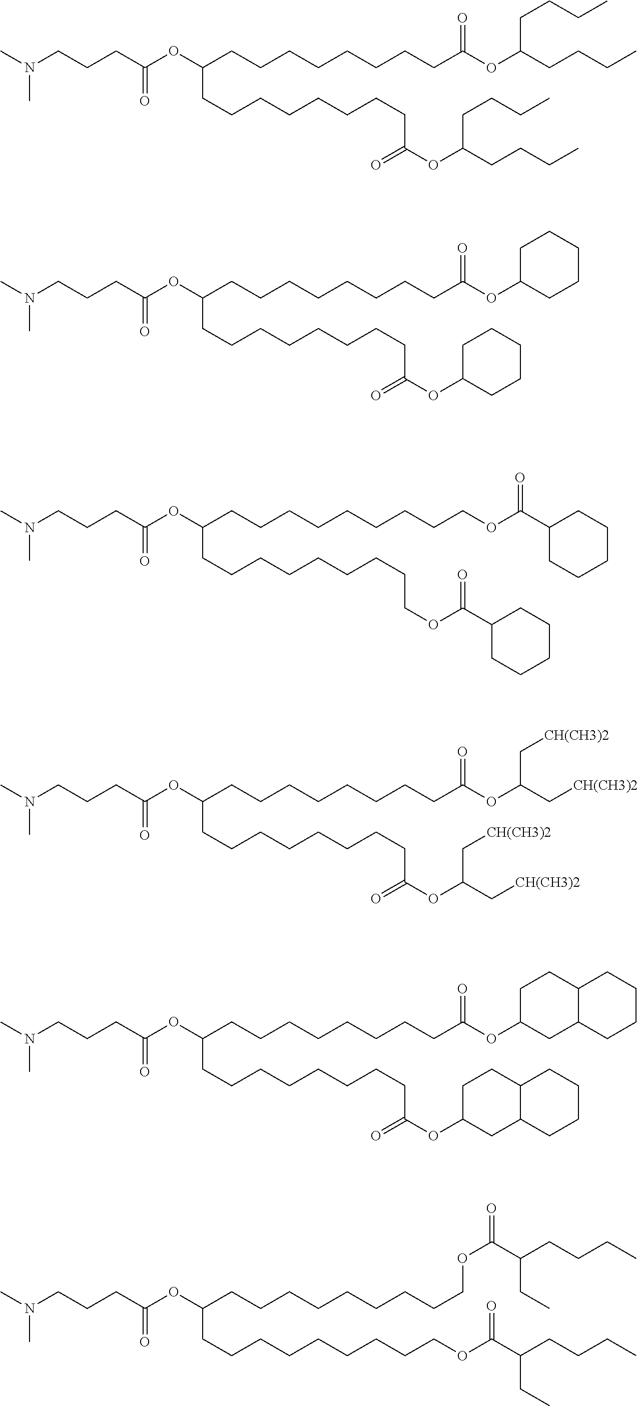

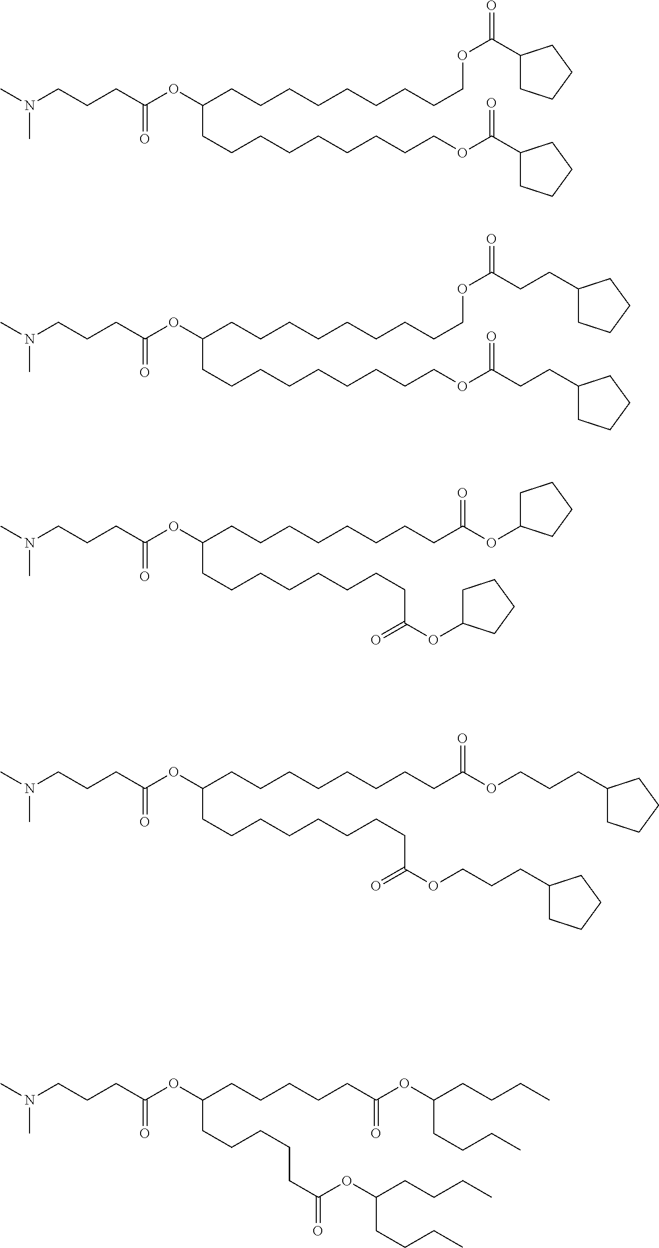 [SMILES: CN(C)CCCC(=O)OC(CCCCCCCCCOC(=O)C1CCCC1)CCCCCCCCCOC(=O)C1CCCC1 ; CN(C)CCCC(=O)OC(CCCCCCCCCOC(=O)CCC1CCCC1)CCCCCCCCCOC(=O)CCC1CCCC1 ; CN(C)CCCC(=O)OC(CCCCCCCCC(=O)OC1CCCC1)CCCCCCCCC(=O)OC1CCCC1 ; CN(C)CCCC(=O)OC(CCCCCCCCC(=O)OCCCC1CCCC1)CCCCCCCCC(=O)OCCCC1CCCC1 ; CCCCC(CCCC)OC(=O)CCCCCC(CCCCCC(=O)OC(CCCC)CCCC)OC(=O)CCCN(C)C]

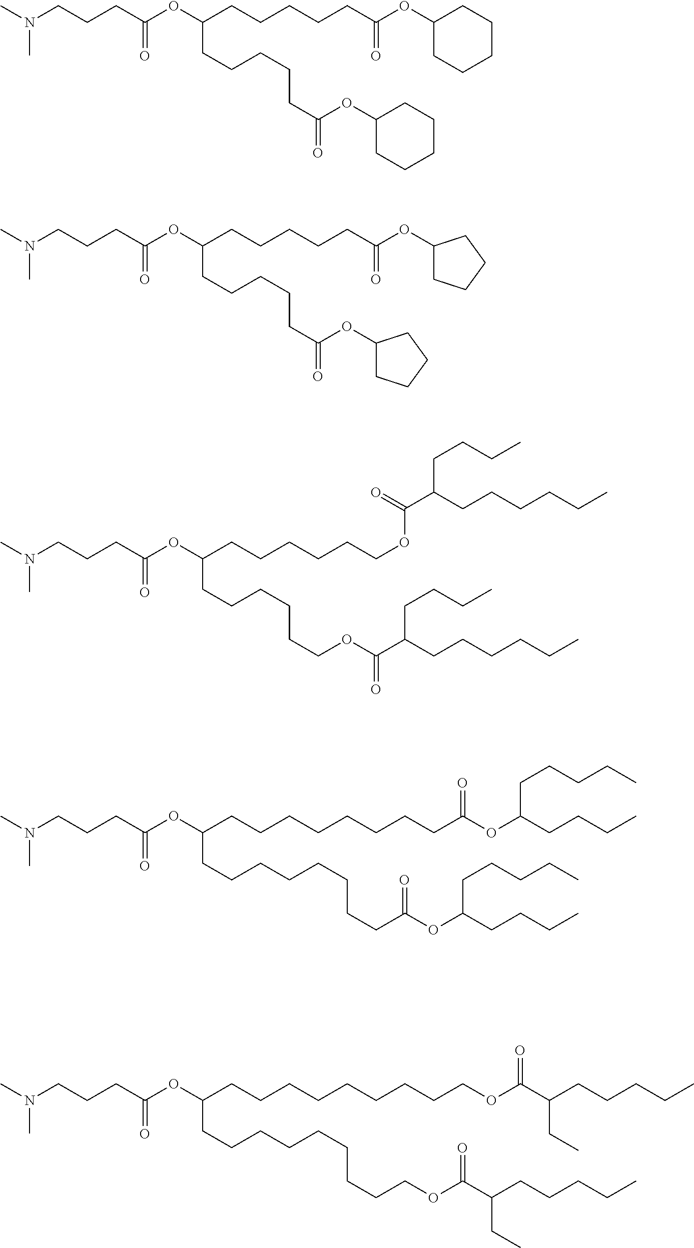

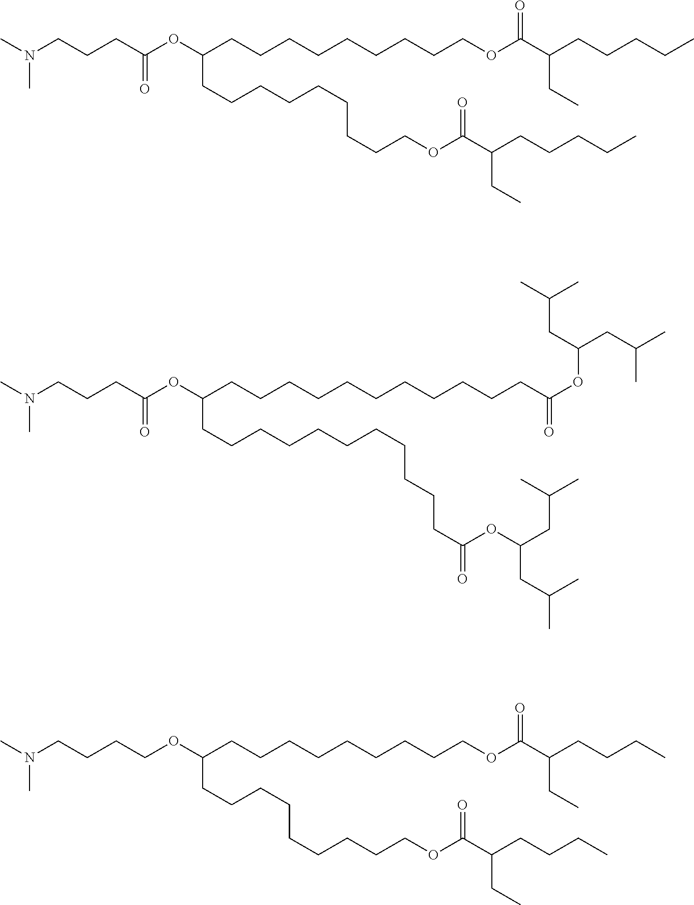

In some embodiments, the lipid disclosed in US 2015/0005363 is selected from the following compounds, and salts thereof (including pharmaceutically acceptable salts thereof):

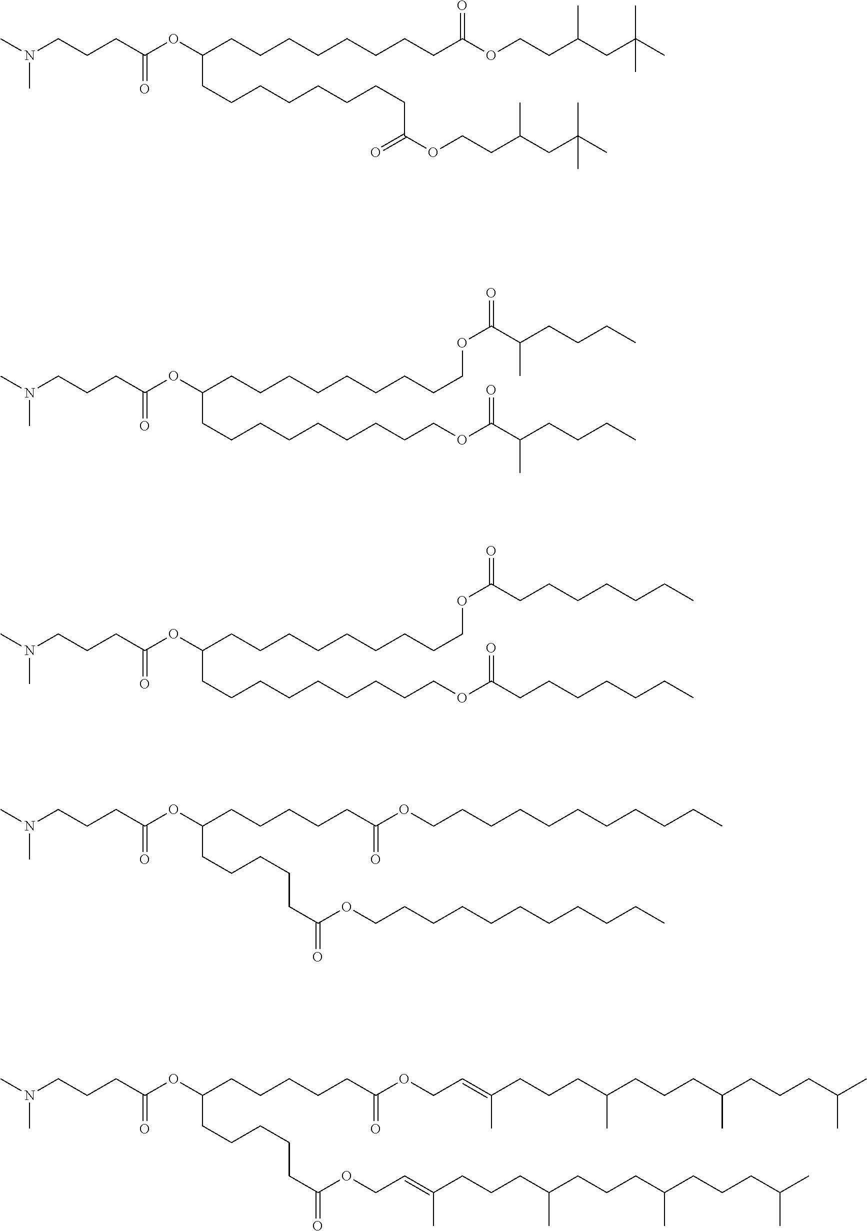

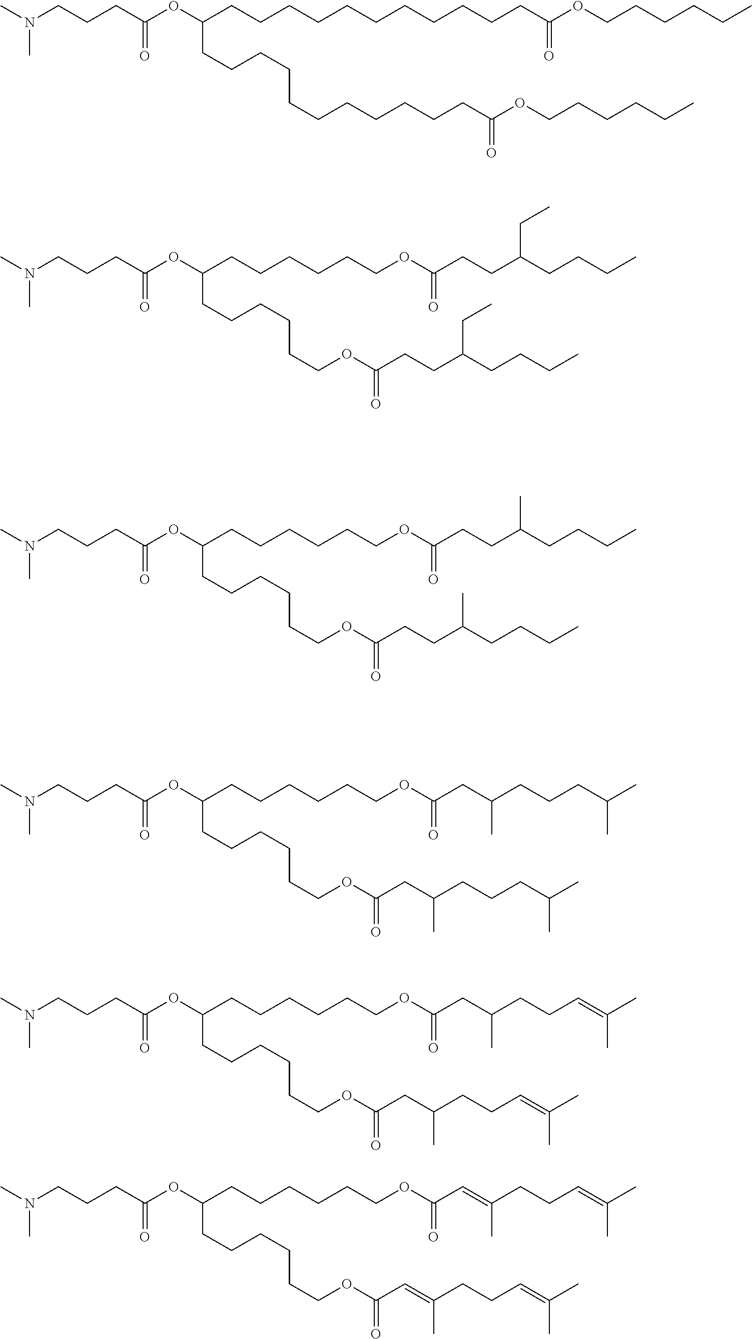

In some embodiments, a LNP described herein comprises a lipid, e.g., an ionizable lipid, disclosed in US 2014/0308304, which is incorporated herein by reference in its entirety.

In some embodiments, the lipid disclosed in US 2014/0308304 is a compound of formula (I):

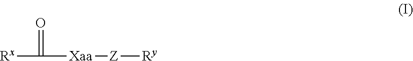

or a salt thereof (e.g., a pharmaceutically acceptable salt thereof), wherein
Xaa is a D- or L-amino acid residue having the formula —NR^{N}—CR¹R²—(C═O)—, or a peptide of amino acid residues having the formula —{NR^{N}—CR¹R²—(C═O)}ₙ—, wherein n is 2 to 20;
R¹ is independently, for each occurrence, a non-hydrogen, substituted or unsubstituted side chain of an amino acid;
R² and R^{N} are independently, for each occurrence, hydrogen, an organic group consisting of carbon, oxygen, nitrogen, sulfur, and hydrogen atoms, or any combination of the foregoing, and having from 1 to 20 carbon atoms, C₍₁₋₅₎alkyl, cycloalkyl, cycloalkylalkyl, C₍₃₋₅₎alkenyl, C₍₃₋₅₎alkynyl, C₍₁₋₅₎alkanoyl, C₍₁₋₅₎alkanoyloxy, C₍₁₋₅₎alkoxy, C₍₁₋₅₎alkoxy-C₍₁₋₅₎alkyl, C₍₁₋₅₎alkoxy-C₍₁₋₅₎alkoxy, C₍₁₋₅₎alkyl-amino-C₍₁₋₅₎alkyl-, C₍₁₋₅₎dialkyl-amino-C₍₁₋₅₎alkyl-, nitro-C₍₁₋₅₎alkyl, cyano-C₍₁₋₅₎alkyl, aryl-C₍₁₋₅₎alkyl, 4-biphenyl-C₍₁₋₅₎alkyl, carboxyl, or hydroxyl;
Z is NH, O, S, —CH₂S—, —CH₂S(O)—, or an organic linker consisting of 1-40 atoms selected from hydrogen, carbon, oxygen, nitrogen, and sulfur atoms (preferably, Z is NH or O);
R^{x} and R^{y} are, independently, (i) a lipophilic tail derived from a lipid (which can be naturally-occurring or synthetic), phospholipid, glycolipid, triacylglycerol, glycerophospholipid, sphingolipid, ceramide, sphingomyelin, cerebroside, or ganglioside, wherein the tail optionally includes a steroid; (ii) an amino acid terminal group selected from hydrogen, hydroxyl, amino, and an organic protecting group; or (iii) a substituted or unsubstituted C₍₃₋₂₂₎alkyl, C₍₆₋₁₂₎cycloalkyl, C₍₆₋₁₂₎cycloalkyl-C₍₃₋₂₂₎alkyl, C₍₃₋₂₂₎alkenyl, C₍₃₋₂₂₎alkynyl, C₍₃₋₂₂₎alkoxy, or C₍₆₋₁₂₎-alkoxy-C₍₃₋₂₂₎alkyl;
one of R^{x} and R^{y} is a lipophilic tail as defined above and the other is an amino acid terminal group, or both R^{x} and R^{y} are lipophilic tails;
at least one of R^{x} and R^{y} is interrupted by one or more biodegradable groups (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)— or

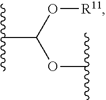

(wherein R¹¹ is a C₂-C₈ alkyl or alkenyl), in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in some embodiments, each occurrence of R³ and R⁴ are, independently H or C₁-C₄ alkyl)); and
R^{x} and R^{y} each, independently, optionally have one or more carbon-carbon double bonds.

In some embodiments, the lipid disclosed in US 2014/0308304 is a compound of formula (IA):

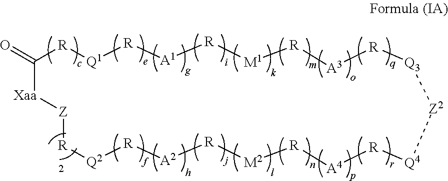

or a salt thereof (e.g., a pharmaceutically acceptable salt thereof), wherein
Z and Xaa are as defined with respect to formula (I) (the variables which are used in the definition of Xaa, namely R^{N}, R¹ and R², are also as defined in formula (I));
each occurrence of R is, independently, —(CR³R⁴)—;
each occurrence of R³ and R⁴ are, independently H, halogen, OH, alkyl, alkoxy, —NH₂, alkylamino, or dialkylamino (in some embodiments, each occurrence of R³ and R⁴ are, independently H or C₁-C₄ alkyl);
or R³ and R⁴, together with the carbon atom to which they are directly attached, form a cycloalkyl group, wherein no more than three R groups in each chain between the —Z-Xaa-C(O)— and Z² moieties are cycloalkyl (e.g., cyclopropyl);
Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;
Q³ and Q⁴ are each, independently, H, —(CR³R⁴)—, cycloalkyl, heterocyclyl, heterocyclylalkyl, aryl, heteroaryl, or a cholesterol moiety;
each occurrence of A¹, A², A³ and A⁴ is, independently, —(CR⁵R⁵—CR⁵═CR⁵)—;
M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

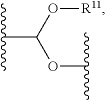

(wherein R¹¹ is a C₂-C₈ alkyl or alkenyl));
each occurrence of R⁵ is, independently, H or alkyl (e.g., C₁-C₄ alkyl);
Z² is absent, alkylene or —O—P(O)(OH)—O—;
each [symbol] attached to Z² is an optional bond, such that when Z² is absent, Q³ and Q⁴ are not directly covalently bound together;
c, d, e, f, i, j, m, n, q and r are each, independently, 0, 1, 2, 3, 4, 5, 6, 7, 8, 9, or 10;
g and h are each, independently, 0, 1 or 2;
k and l are each, independently, 0 or 1, wherein at least one of k and 1 is 1;
and p are each, independently, 0, 1 or 2; and
Q³ and Q⁴ are each, independently, separated from the —Z-Xaa-C(O)— moiety by a chain of 8 or more atoms (e.g., 12 or 14 or more atoms).

In some embodiments the lipids disclosed in US 2014/0308304 are of the formula (IC):

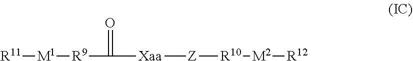

or a salt thereof (e.g., a pharmaceutically acceptable salt thereof), wherein
Z and Xaa are as defined with respect to formula (I) (the variables which are used in the definition of Xaa, namely R^{N}, R¹ and R², are also as defined in formula (I));
each of R⁹ and R¹⁰ are, independently, alkylene or alkenylene;
each of R¹¹ and R¹² are, independently, alkyl or alkenyl, optionally terminated by COOR¹³ wherein each R¹³ is independently unsubstituted alkyl (e.g., C₁-C₄ alkyl such as methyl or ethyl), substituted alkyl (such as benzyl), or cycloalkyl;
M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

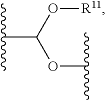

(wherein R¹¹ is a C₂-C₈ alkyl or alkenyl), in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in some embodiments, each occurrence of R³ and R⁴ are, independently H or C₁-C₄ alkyl));
R⁹, M¹, and R¹¹ are together at least 8 carbon atoms in length (e.g., 12 or 14 carbon atoms or longer); and
R¹⁰, M², and R¹² are together at least 8 carbon atoms in length (e.g., 12 or 14 carbon atoms or longer).

In some embodiments, the lipid disclosed in US 2014/0308304 is a compound of the formula II:

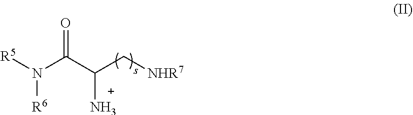

or a salt thereof (e.g., a pharmaceutically acceptable salt thereof), wherein:
s is 1, 2, 3 or 4; and
R⁷ is selected from lysyl, ornithyl, 2,3-diaminobutyryl, histidyl and an acyl moiety of the formula:

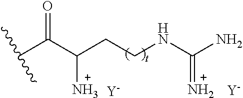

t is 1, 2 or 3;
the NH₃⁺ moiety in the acyl moiety in R⁷ is optionally absent;
each occurrence of Y⁻ is independently a pharmaceutically acceptable anion (e.g., halide, such as chloride);
R⁵ and R⁶ are each, independently a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid; or a substituted or unsubstituted C₍₃₋₂₂₎alkyl, C₍₆₋₁₂₎cycloalkyl, C₍₆₋₁₂₎cycloalkyl-C₍₃₋₂₂₎alkyl, C₍₃₋₂₂₎alkenyl, C₍₃₋₂₂₎alkynyl, C₍₃₋₂₂₎alkoxy, or C₍₆₋₁₂₎alkoxy-C₍₃₋₂₂₎alkyl;
at least one of R⁵ and R⁶ is interrupted by one or more biodegradable groups (e.g., —SC(O)—, —C(O)S—, —OC(S)—, —C(S)O—, —S—S—, —C(O)(NR^{a})—, —N(R^{a})C(O)—, —C(S)(NR^{a})—, —N(R^{a})C(O)—, —N(R^{a})C(O)N(R^{a})—, or —OC(O)O—);
each occurrence of R^{a} is, independently, H or alkyl; and
R⁵ and R⁶ each, independently, optionally contain one or more carbon-carbon double bonds.

In some embodiments, the lipids disclosed in US 2014/0308304 are of the formula (IIA):

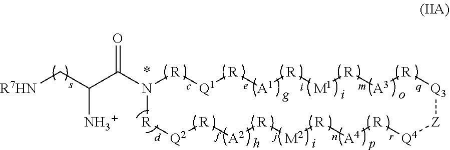

or a salt thereof (e.g., a pharmaceutically acceptable salt thereof), wherein:
R⁷ and s are as defined with respect to formula (II);
each occurrence of R is, independently, —(CR³R⁴)—;
each occurrence of R³ and R⁴ are, independently H, halogen, OH, alkyl, alkoxy, —NH₂, alkylamino, or dialkylamino (in some embodiments, each occurrence of R³ and R⁴ are, independently H or C₁-C₄ alkyl);
or R³ and R⁴, together with the carbon atom to which they are directly attached, form a cycloalkyl group, wherein no more than three R groups in each chain attached to the nitrogen N* are cycloalkyl (e.g., cyclopropyl);
Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;
Q³ and Q⁴ are each, independently, H, —(CR³R⁴)—, aryl, cycloalkyl, heterocyclyl, heterocyclylalkyl, heteroaryl, or a cholesterol moiety;
each occurrence of A¹, A², A³ and A⁴ is, independently, —(CR⁵R⁵—CR⁵═CR⁵)—;
M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

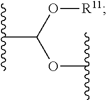

(wherein R¹¹ is a C₂-C₈ alkyl or alkenyl));
each occurrence of R⁵ is, independently, H or alkyl;
Z is absent, alkylene or —O—P(O)(OH)—O—;
each [symbol] attached to Z is an optional bond, such that when Z is absent, Q³ and Q⁴ are not directly covalently bound together;
c, d, e, f, i, j, m, n, q and r are each, independently, 0, 1, 2, 3, 4, 5, 6, 7, 8, 9, or 10;
g and h are each, independently, 0, 1 or 2;
k and 1 are each, independently, 0 or 1, where at least one of k and 1 is 1; and
and p are each, independently, 0, 1 or 2.

In some embodiments the lipid disclosed in US 2014/0308304 are of the formula (IIC):

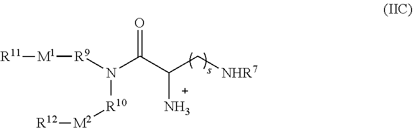

or a salt thereof (e.g., a pharmaceutically acceptable salt thereof), wherein:
R⁷ and s are as defined with respect to formula (II);
each of R⁹ and R¹⁰ are independently alkyl (e.g., C₁₂-C₂₄ alkyl) or alkenyl (e.g., C₁₂-C₂₄ alkenyl);
each of R¹¹ and R¹² are independently alkyl or alkenyl, optionally terminated by COOR¹³ where each R¹³ is independently alkyl (e.g., C₁-C₄ alkyl such as methyl or ethyl);
M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

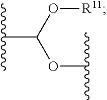

(wherein R¹¹ is a C₂-C₈ alkyl or alkenyl); in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in some embodiments, each occurrence of R³ and R⁴ are, independently, H or C₁-C₄ alkyl));
R⁹, M¹, and R¹¹ are together at least 8 carbons atoms in length (e.g., 12 or 14 carbon atoms or longer); and
R¹⁰, M², and R¹² are together at least 8 carbons atoms in length (e.g., 12 or 14 carbon atoms or longer).

In some embodiments, the lipid disclosed in US 2014/0308304 is a compound of the formula (4):

wherein:

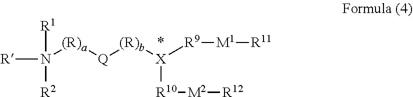

X is N or P;
R¹, R², R, a, b, M¹, and M² are as defined with respect to formula (I);
Q is absent or is —O—, —NH—, —S—, —C(O)O—, —OC(O)—, —C(O)N(R⁴)—, —N(R⁵)C(O)—, —S—S—, OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;
R′ is absent, hydrogen, or alkyl (e.g., C₁-C₄ alkyl);
each of R⁹ and R¹⁰ are independently alkylene, or alkenylene; and
each of R¹¹ and R¹² are independently alkyl or alkenyl, optionally terminated by COOR¹³ where each R¹³ is independently alkyl (e.g., C₁-C₄ alkyl such as methyl or ethyl);
R⁹, M¹, and R¹¹ are together at least 8 carbons atoms in length (e.g., 12 or 14 carbon atoms or longer); and
R¹⁰, M², and R¹² are together at least 8 carbons atoms in length (e.g., 12 or 14 carbon atoms or longer).

In some embodiments, the lipid disclosed in US 2014/0308304 is a compound of the formula (5)

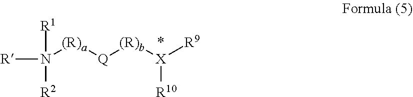

wherein:
X is N or P;
R¹, R², R, a, and b are as defined with respect to formula (I);
Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—; R′ is absent, hydrogen, or alkyl (e.g., C₁-C₄ alkyl);
each of R⁹ and R¹⁰ are independently C₁₂-C₂₄ alkyl or alkenyl substituted at its terminus with a biodegradable group, such as —COOR¹³ where each R¹³ is independently alkyl (preferably C₁-C₄ alkyl such as methyl or ethyl).

In some embodiments the lipids disclosed in US 2014/0308304 are of Formula A:

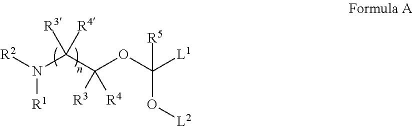

or a pharmaceutically acceptable salt or stereoisomer thereof, wherein:
n is 0-6 (e.g., n is 0, 1 or 2);
R¹ and R² are independently selected from H, (C₁-C₆)alkyl, heterocyclyl, and a polyamine, wherein said alkyl, heterocyclyl and polyamine are optionally substituted with one or more substituents selected from R′,
or R¹ and R² can be taken together with the nitrogen to which they are attached to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;
R³ is selected from H and (C₁-C₆)alkyl, wherein said alkyl is optionally substituted with one or more substituents selected from R′, or R³ can be taken together with R′ to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;
each occurrence of R⁴, R^{3′}, and R^{4′} is independently selected from H, (C₁-C₆)alkyl and O-alkyl, said alkyl is optionally substituted with one or more substituents selected from R′; or R^{3′} and R^{4′} when directly bound to the same carbon atom form an oxo (═O) group, cyclopropyl or cyclobutyl;
or R³ and R⁴ form an oxo (═O) group;
R⁵ is selected from H and (C₁-C₆)alkyl; or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;
each occurrence of R′ is independently selected from halogen, R″, OR″, SR″, CN, CO₂R″ and CON(R″)₂;
each occurrence of R″ is selected from H and (C₁-C₆)alkyl, wherein said alkyl is optionally substituted with one or more substituents selected from halogen and OH;
L¹ is a C₄-C₂₂ alkyl or C₄-C₂₂ alkenyl, said alkyl or alkenyl is optionally interrupted by or terminated with one or more biodegradable groups; and said alkyl or alkenyl is optionally substituted with one or more substituents selected from R′; and
L² is a C₄-C₂₂ alkyl or C₄-C₂₂ alkenyl, said alkyl or alkenyl is optionally interrupted by or terminated with one or more biodegradable groups; and said alkyl or alkenyl is optionally substituted with one or more substituents selected from R′;
with the proviso that the CR^{3′}R^{4′} group when present adjacent to the nitrogen atom in formula A is not a ketone (—C(O)—).

In some embodiments the lipids disclosed in US 2014/0308304 are of formula B:

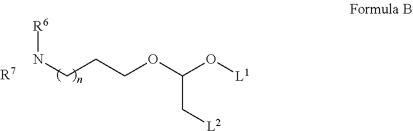

or a pharmaceutically acceptable salt or stereoisomer thereof, wherein:
n is 0, 1, 2, 3, 4, or 5;
R⁶ and R⁷ are each independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;
L¹ is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;
each occurrence of R′ is independently selected from halogen, R″, OR″, SR″, CN, CO₂R″ and CON(R″)₂; and
each occurrence of R″ is independently selected from H and (C₁-C₆)alkyl, wherein said alkyl is optionally substituted with one or more substituents selected from halogen and OH.

In some embodiments, lipid disclosed in US 2014/0308304 are of formula C:

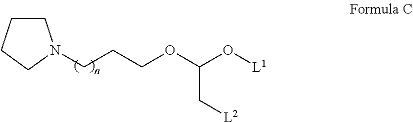

or a pharmaceutically acceptable salt or stereoisomer thereof, wherein:
n is 0, 1, 2, 3, 4, or 5;
L¹ is a C₄-C₂₂ alkyl or C₄-C₂₂ alkenyl, said alkyl or alkenyl optionally has one or more biodegradable groups; each biodegradable group independently interrupts the alkyl or alkenyl group or is substituted at the terminus of the alkyl or alkenyl group, and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;
each occurrence of R′ is independently selected from halogen, R″, OR″, SR″, CN, CO₂R″ and CON(R″)2; and
each occurrence of R″ is independently selected from H and (C₁-C₆)alkyl, wherein said alkyl is optionally substituted with one or more substituents selected from halogen and OH.

In some embodiments, the lipid disclosed in US 2014/0308304 are of formula D:

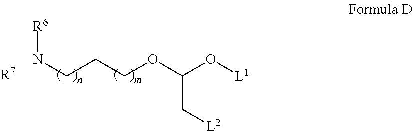

or a pharmaceutically acceptable salt or stereoisomer thereof, wherein
m is 0, 1, 2, or 3;
n is 0, 1, 2, 3, 4, or 5;
R⁶ and R⁷ are each independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;
L¹ is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;
each occurrence of R′ is independently selected from halogen, R″, OR″, SR″, CN, CO₂R″ and CON(R″)₂; and
each occurrence of R″ is independently selected from H and (C₁-C₆)alkyl, wherein said alkyl is optionally substituted with one or more substituents selected from halogen and OH.

In some embodiments lipid disclosed in US 2014/0308304 are of formula E:

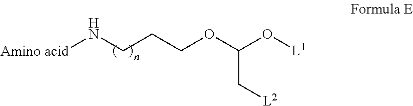

or a pharmaceutically acceptable salt or stereoisomer thereof, wherein
n is 0, 1, 2, 3, 4, or 5;
the group “amino acid” is an amino acid residue;
L¹ is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 substituents selected from R′; and
L² is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 substituents selected from R′;
each occurrence of R′ is independently selected from halogen, R″, OR″, SR″, CN, CO₂R″ and CON(R″)2; and
each occurrence of R″ is independently selected from H and (C₁-C₆)alkyl, wherein said alkyl is optionally substituted with one or more substituents selected from halogen and OH.

The amino acid residue in formula E may have the formula —C(O)—C(R⁹)(NH₂), where R⁹ is an amino acid side chain.

In some embodiments, the lipid disclosed in US 2014/0308304 are of formula F:

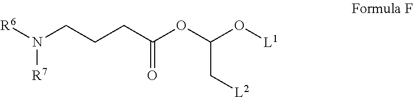

or a pharmaceutically acceptable salt or stereoisomer thereof, wherein:
R⁶ and R⁷ are independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;
L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and
L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;
each occurrence of R′ is independently selected from halogen, R″, OR″, SR″, CN, CO₂R″ and CON(R″)2;
each occurrence of R″ is independently selected from H and (C₁-C₆)alkyl, wherein said alkyl is optionally substituted with one or more substituents selected from halogen and OH.

In some embodiments, the lipid disclosed in US 2014/0308304 are of formula G:

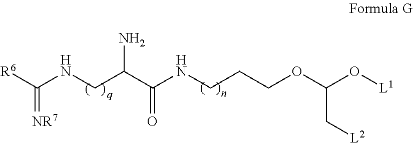

or a pharmaceutically acceptable salt or stereoisomer thereof, wherein:
n is 0, 1, 2, 3, 4, or 5;
q is 1, 2, 3, or 4
R⁶ and R⁷ are independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl);
L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and
L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;
each occurrence of R′ is independently selected from halogen, R″, OR″, SR″, CN, CO₂R″ and CON(R″)2;
each occurrence of R″ is independently selected from H and (C₁-C₆)alkyl, wherein said alkyl is optionally substituted with one or more substituents selected from halogen and OH.

In some embodiments, a LNP described herein comprises a lipid, e.g., an ionizable lipid, disclosed in US 2013/0053572, which is incorporated herein by reference in its entirety.

In some embodiments, the lipids disclosed in US 2013/0053572 are of Formula A:

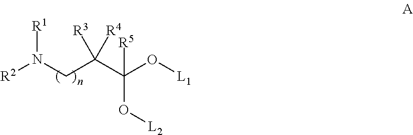

wherein:
n is 0, 1 or 2;
R¹ and R² are independently selected from H, (C₁-C₆)alkyl, heterocyclyl, and a polyamine, wherein said alkyl, heterocyclyl and polyamine are optionally substituted with one or more substituents selected from R′, or R′, and R² can be taken together with the nitrogen to which they are attached to form a monocyclic heterocycle with 4-7 members optionally containing, in addition to the nitrogen, one or two additional heteroatoms selected from N, O and S, said monocyclic heterocycle is optionally substituted with one or more substituents selected from R′;
R³ is selected from H and (C₁-C₆)alkyl, wherein said alkyl is optionally substituted with one or more substituents selected from R′, or R³ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;
R⁴ is selected from H, (C₁-C₆)alkyl and O-alkyl, said alkyl is optionally substituted with one or more substituents selected from R′;
R⁵ is selected from H and (C₁-C₆)alkyl; or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;
R′ is independently selected from halogen, R″, OR″, CN, CO₂R″ and CON(R″)₂;
R″ is selected from H and (C₁-C₆)alkyl, wherein said alkyl is optionally substituted with one or more substituents selected from halogen and OH;
L₁ is a C₄-C₂₂ alkenyl, said alkenyl is optionally substituted with one or more substituents selected from R′; and
L2 is a C₄-C₂₂ alkenyl, said alkenyl is optionally substituted with one or more substituents selected from R′;
or any pharmaceutically acceptable salt or stereoisomer thereof.

In some embodiments, a LNP described herein comprises a lipid, e.g., an ionizable lipid, disclosed in US Application publication US2017/0119904, which is incorporated by reference herein, in its entirety.

In some embodiments, a LNP described herein comprises a lipid, e.g., an ionizable lipid, disclosed in PCT Application publication WO2021/204179, which is incorporated by reference herein, in its entirety.

In some embodiments, the ionizable lipid is Compound 1 or Compound 2:

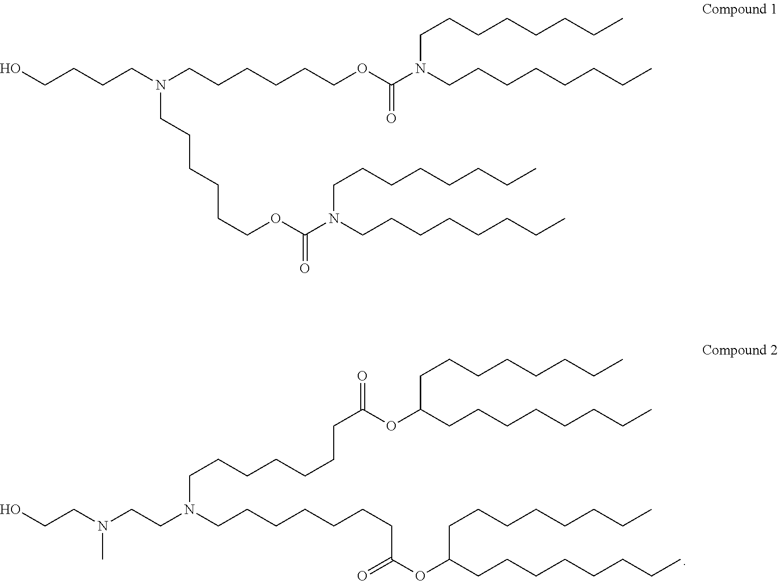 [SMILES: CCCCCCCCN(CCCCCCCC)C(=O)OCCCCCCN(CCCCO)CCCCCCOC(=O)N(CCCCCCCC)CCCCCCCC ; CCCCCCCCC(CCCCCCCC)OC(=O)CCCCCCCN(CCCCCCCC(=O)OC(CCCCCCCC)CCCCCCCC)CCN(C)CCO]

ii. Structural Lipids

In some embodiments, a LNP comprises a structural lipid. Structural lipids can be selected from the group consisting of, but are not limited to, cholesterol, fecosterol, beta sitosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occurring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

iii. PEGylated Lipids

In some embodiments, a LNP comprises a PEGylated lipid or PEG-modified lipid. A PEGylated lipid is a lipid modified with polyethylene glycol. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, the lipid disclosed in US 2021/0087135 is a compound of Formula (PL-I):

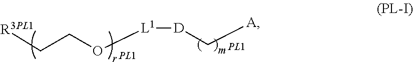

or a salt thereof, wherein:
R^{3PL1} is —OR^{OPL1}.
R^{OPL1} is hydrogen, optionally substituted alkyl, or an oxygen protecting group;
r^{PL1} is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{NPL1}), S, C(O), C(O)N(R^{NPL1}), NR^{NPL1}C(O), C(O)O, OC(O), OC(O)O, OC(O)N(R^{NPL1}), NR^{NPL1}C(O)O or NR^{NPL1}C(O)N(R^{NPL1});
D is a moiety obtained by click chemistry or a moiety cleavable under physiological conditions;
m^{PL1} is 0, 1, 2, 3, 4, 5, 6, 7, 8, 9, or 10;
A is of the formula:

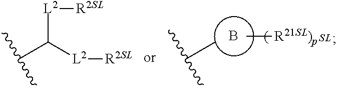

each instance of L² is independently a bond or optionally substituted C₁₋₆ alkylene, wherein one methylene unit of the optionally substituted C₁₋₆ alkylene is optionally replaced with O, N(R^{NPL1}), S, C(O), C(O)N(R^{NPL1}), NR^{NPL1}C(O), C(O)O, OC(O), OC(O)O, OC(O)N(R^{NPL1}), NR^{NPL1}C(O)O, or NR^{NPL1}C(O)N(R^{NPL1});
each instance of R^{2SL} is independently optionally substituted C₁₋₃₀ alkyl, optionally substituted C₁₋₃₀ alkenyl, or optionally substituted C₁₋₃₀ alkynyl; optionally wherein one or more methylene units of R^{2SL} are independently replaced with optionally substituted carbocyclylene, optionally substituted heterocyclylene, optionally substituted arylene, optionally substituted heteroarylene, N(R^{NPL1}), O, S, C(O), C(O)N(R^{NPL1}), NR^{NPL1}C(O), NR^{NPL1}C(O)N(R^{NPL1}), —C(O)O, OC(O), OC(O)O, OC(O)N(R^{NPL1}), NR^{NPL1}C(O)O, C(O)S, SC(O), C(═NR^{NPL1}), —C(═NR^{NPL1})N(R^{NPL1}), NR^{NPL1}C(═NR^{NPL1}), NR^{NPL1}C(NR^{NPL1})N(R^{NPL1}), C(S), C(S)N(R^{NPL1}), NR^{NPL1}C(S), NR^{NPL1}C(S)N(R^{NPL1}), S(O), OS(O), S(O)O, OS(O)O, OS(O)₂, S(O)₂O, OS(O)₂O, N(R^{NPL1})S(O), S(O)N(R^{NPL1}), N(R^{NPL1})S(O)N(R^{NPL1}), OS(O)N(R^{NPL1}), N(R^{NPL1})S(O)O, S(O)₂, N(R^{NPL1})S(O)₂, S(O)₂N(R^{NPL1}), N(R^{NPL1})S(O)₂N(R^{NPL1}), OS(O)₂N(R^{NPL1}) or N(R^{NPL1})S(O)₂₀;
each instance of R^{NPL1} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p^{SL} is 1 or 2.

In some embodiments, the lipid disclosed in US 2021/0087135 is a compound of Formula (PL-I-OH):

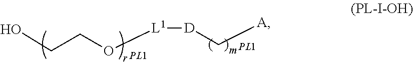

or a salt thereof.

In some embodiments, the lipid disclosed in US 2021/0087135 is a compound of Formula (PL-II-OH):

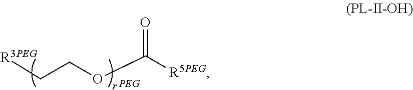

or a salt or isomer thereof, wherein:
R^{3PEG} is —OR^{O};
R^{O} is hydrogen, C₁₋₆ alkyl or an oxygen protecting group;
r^{PEG} is an integer between 1 and 100;
R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{NPEG})—, —O—, —S—, —C(O)—, —C(O)N(R^{NPEG})—, —NR^{NPEG}C(O)—, —NR^{NPEG}C(O)N(R^{NPEG})—, —C(O)O, —OC(O)—, —OC(O)O—, —OC(O)N(R^{NPEG})—, —NR^{NPEG}C(O)O—, —C(O)S—, —SC(O)—, —C(═NR^{NPEG})—, —C(═NR^{NPEG})N(R^{NPEG})—, —NR^{NPEG}C(═NR^{NPEG})—, —NR^{NPEG}C(═NR^{NPEG})N(R^{NPEG})—, —C(S)—, —C(S)N(R^{NPEG})—, —NR^{NPEG}C(S)—, —NR^{NPEG}C(S)N(R^{NPEG})—, —S(O)—, —OS(O)O—, —S(O)O—, —OS(O)O—, —OS(O)₂—, —S(O)₂O—, —OS(O)₂O—, —N(R^{NPEG})S(O)—, —S(O)N(R^{NPEG})—, —N(R^{NPEG})S(O)N(R^{NPEG})—, —OS(O)N(R^{NPEG}), N(R^{NPEG})S(O)O—, —S(O)₂—, —N(R^{NPEG})S(O)₂—, —S(O)₂N(R^{NPEG})—, —N(R^{NPEG})S(O)₂N(R^{NPEG})—, —OS(O)₂N(R^{NPEG})—, or —N(R^{NPEG})S(O)₂O—; and
each instance of R^{NPEG} is independently hydrogen, C₁₋₆ alkyl, or a nitrogen protecting group.

In some embodiments, the lipid disclosed in US 2021/0087135 is a compound of Formula (PL-II):

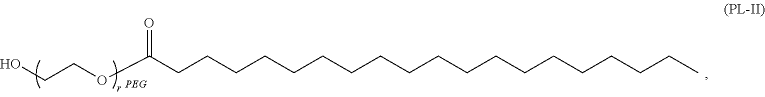

wherein r^{PEG} is an integer between 1 and 100.

In some embodiments, the PEG lipids disclosed in US 2021/0128488 are of structure (II):

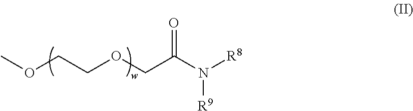

or a pharmaceutically acceptable salt, tautomer or stereoisomer thereof, wherein:
R⁸ and R⁹ are each independently a straight or branched, alkyl, alkenyl or alkynyl containing from 10 to 30 carbon atoms, wherein the alkyl, alkenyl or alkynyl is optionally interrupted by one or more ester bonds; and
w has a mean value ranging from 30 to 60.

Representative PEG lipids disclosed in US 2013/0195920 include, but are not limited to:

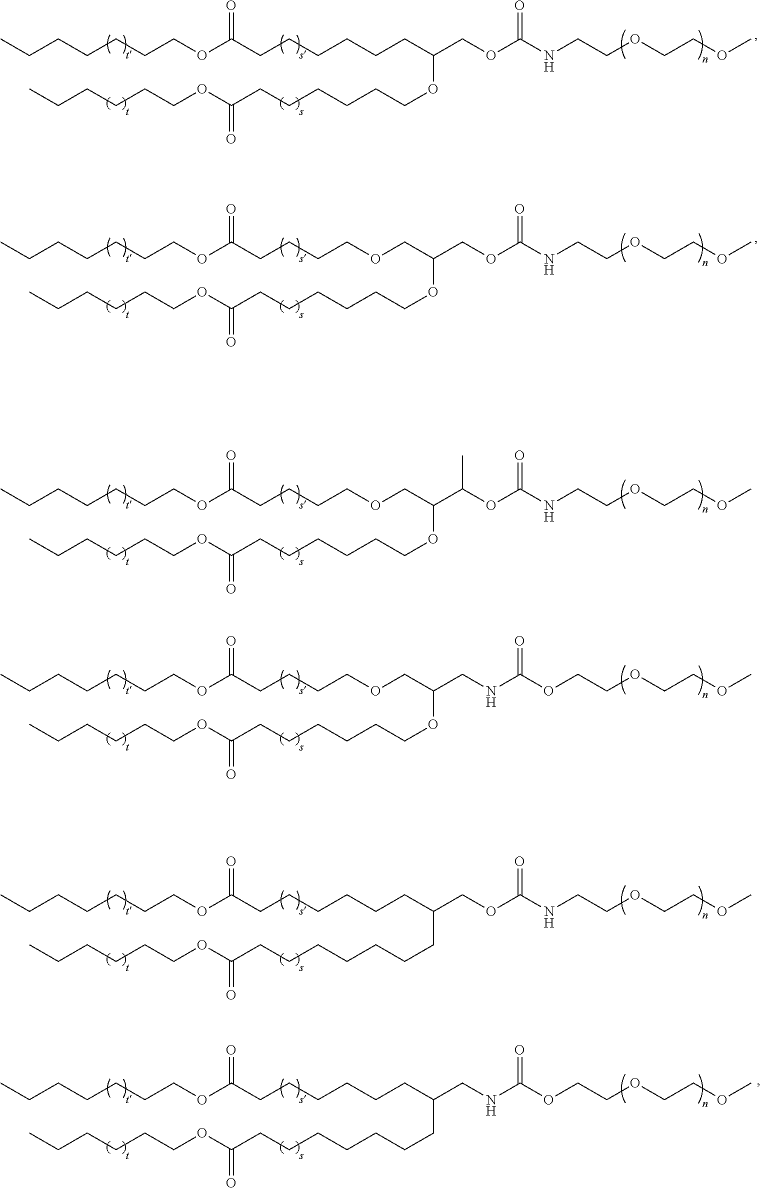

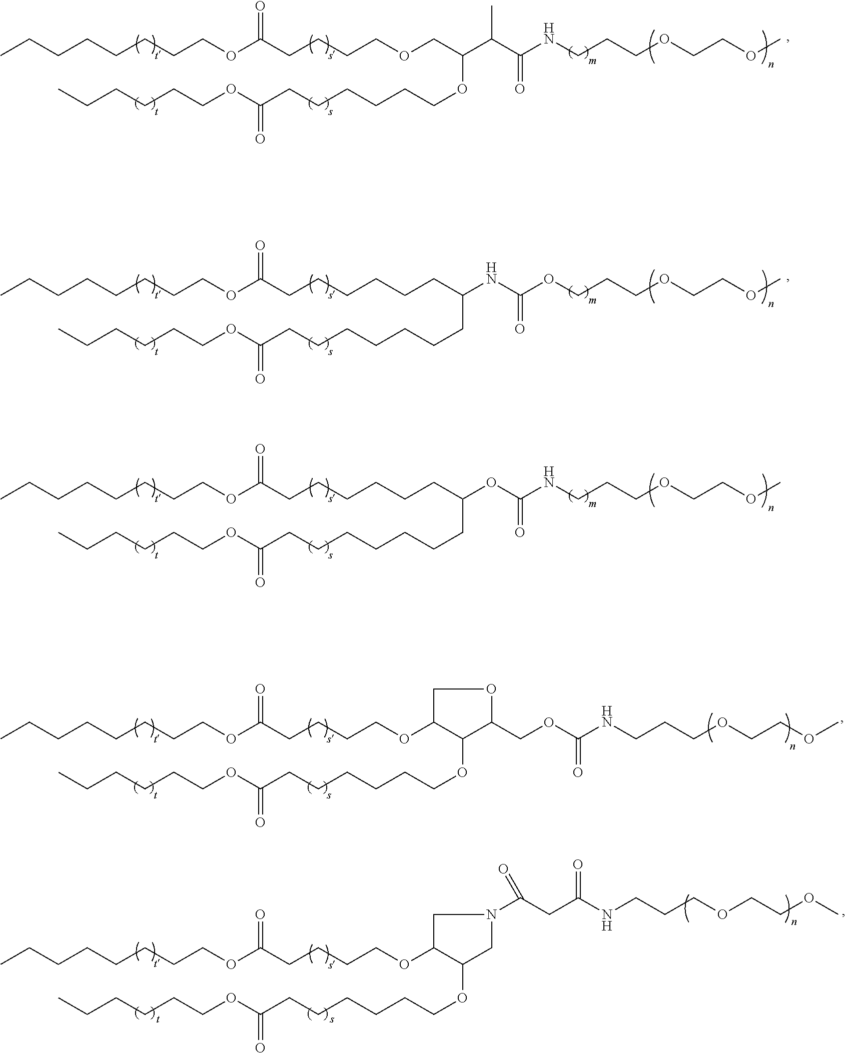

wherein:
n is an integer from 10 to 100 (e.g. 20-50 or 40-50);
s, s′, t and t′ are independently 0, 1, 2, 3, 4, 5, 6 or 7; and m is 1, 2, 3, 4, 5, or 6.

Other representative PEG lipids include, but are not limited to:

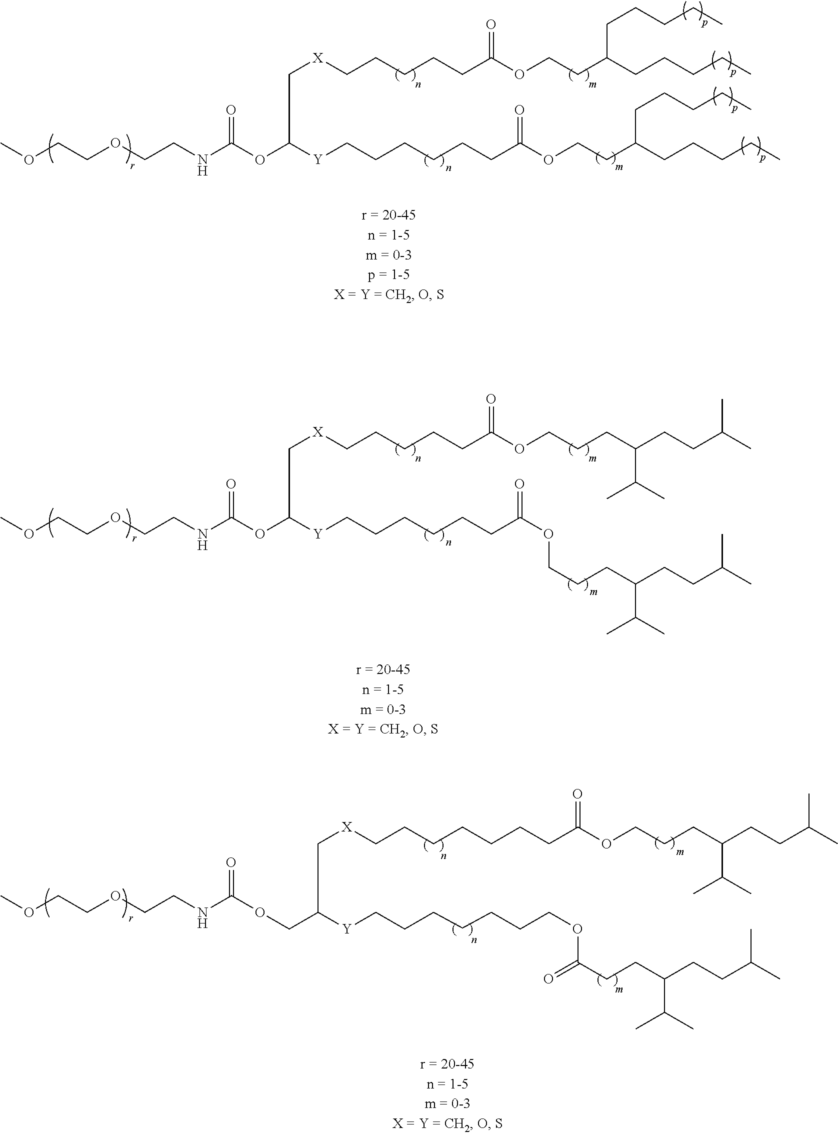

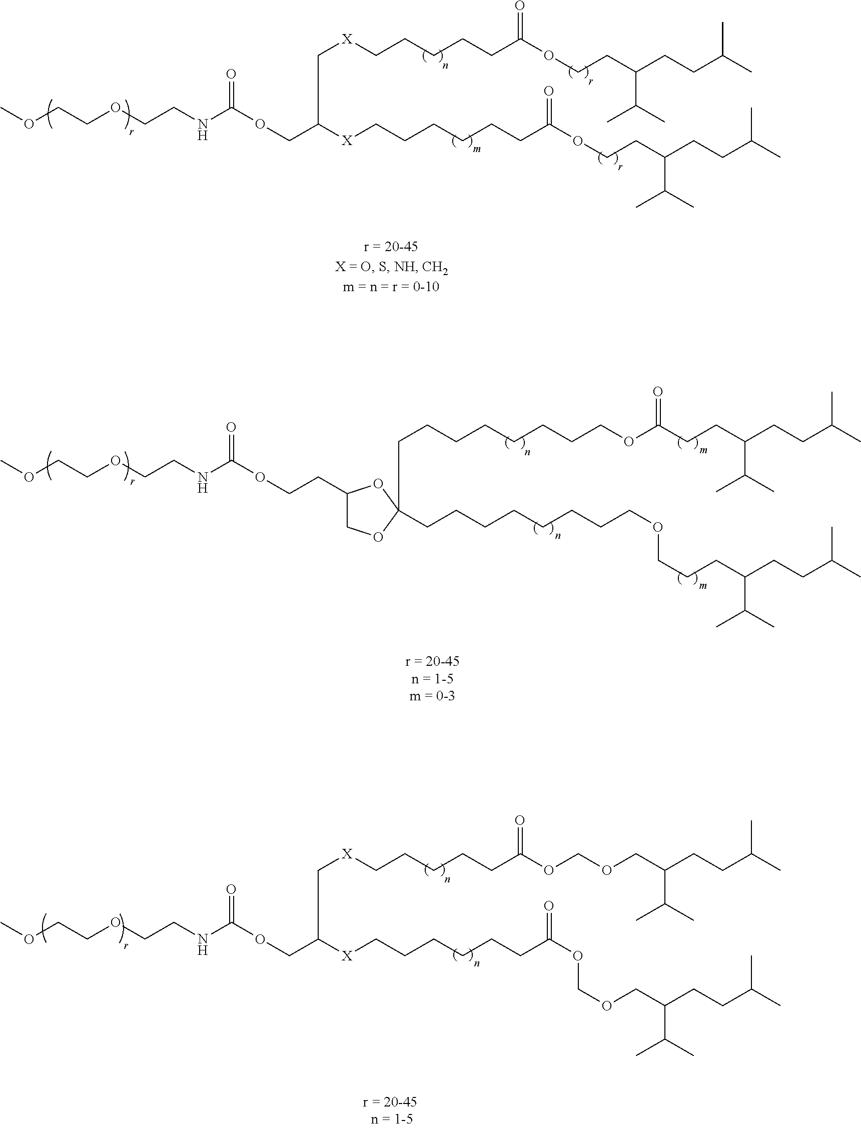

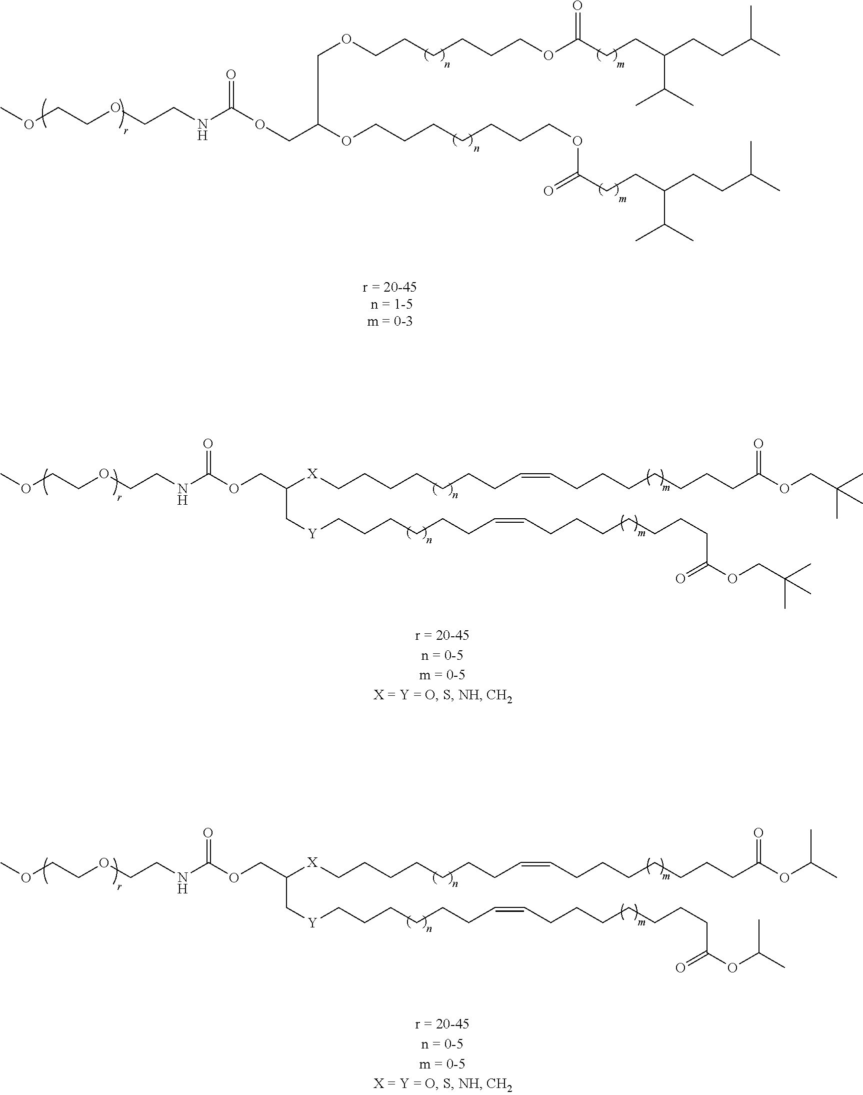

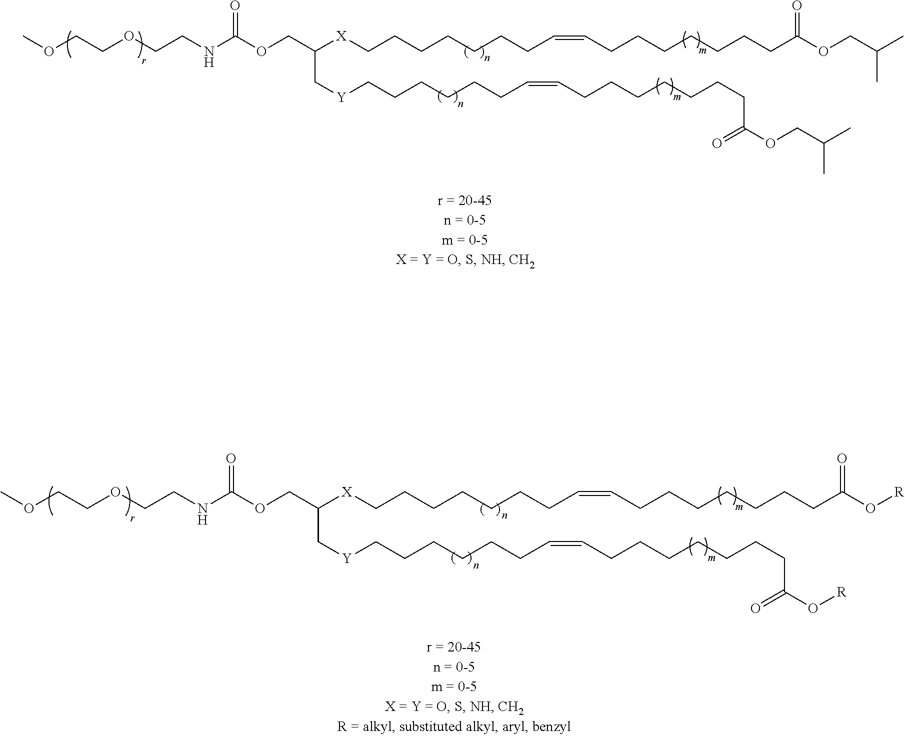

iv. Phospholipids

In some embodiments, a LNP comprises a phospholipid. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanol amine, 1,2-dioleoyl-sn-glycero-3-phospho-rac-(1-glycerol) sodium salt (DOPG), and sphingomyelin. In some embodiments, a LNP includes DSPC. In certain embodiments, a LNP includes DOPE. In some embodiments, a LNP includes both DSPC and DOPE.

In some embodiments, a phospholipid tail may be modified in order to promote endosomal escape as described in U.S. Patent Application 20210121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, phospholipids disclosed in US 2020/0121809 have the following structure:

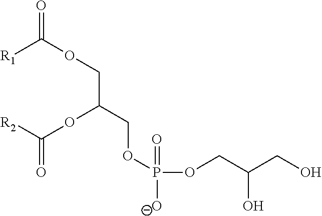

wherein R₁ and R₂ are each independently a branched or straight, saturated or unsaturated carbon chain (e.g., alkyl, alkenyl, alkynyl).
   v. Targeting Moieties

The pharmaceutical composition may be comprised of a polynucleotide, a lipid nanoparticle, and a targeting moiety. The targeting moiety may be an antibody or a fragment thereof. The targeting moiety may be capable of binding to a target antigen.

In some embodiments, the pharmaceutical composition further comprises a targeting moiety.

In some embodiments, the pharmaceutical composition comprises a targeting moiety that is operably connected to a lipid nanoparticle.

In some embodiments, the targeting moiety is capable of binding to a target antigen.

In some embodiments, the target antigen is expressed in the target organ.

In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the lipid nanoparticles may be targeted when conjugated/attached/associated with a targeting moiety such as an antibody.

vi. Zwitterionic Amino Lipids

In some embodiments, a LNP may comprise a zwitterionic lipid. In some embodiments, a LNP comprising a zwitterionic lipid does not comprise a phospholipid.

Zwitterionic amino lipids have been shown to be able to self-assemble into LNPs without phospholipids to load, stabilize, and release mRNAs intracellular as described in U.S. Patent Application 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destabilizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine α-amine may reduce immunogenicity as described in Walsh et al., Synthesis, Characterization and Evaluation of Ionizable Lysine-Based Lipids for siRNA Delivery, Bioconjug Chem. (2013).

vii. Polynucleotides

In some embodiments, a LNP contains a therapeutic agent. In some embodiments, a therapeutic agent is a polynucleotide. In some embodiments, a LNP is capable of delivering a polynucleotide to a target organ. A polynucleotide, in its broadest sense of the term, includes any compound and/or Substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. RNAs useful in the compositions and methods described herein can be selected from the group consisting of but are not limited to, shortimers, antagomirs, antisense, ribozymes, short interfering RNA (siRNA), asymmetrical interfering RNA (aiRNA), microRNA (miRNA), Dicer substrate RNA (dsRNA), short hairpin RNA (shRNA), transfer RNA (tRNA), messenger RNA (mRNA), and mixtures thereof. In some embodiments, a polynucleotide is mRNA. In some embodiments, a polynucleotide is circular RNA. In some embodiments, a polynucleotide encodes a protein. A polynucleotide may encode any polypeptide of interest, including any naturally or non-naturally occurring or otherwise modified polypeptide. A polypeptide may be of any size and may have any secondary structure or activity. In some embodiments, a polypeptide encoded by an mRNA may have a therapeutic effect when expressed in a cell.

In other embodiments, a polynucleotide is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, a polynucleotide is an shRNA or a vector or plasmid encoding the same. An shRNA may be produced inside a target cell upon delivery of an appropriate construct to the nucleus. Constructs and mechanisms relating to shRNA are well known in the relevant arts.

A polynucleotide may include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, a polynucleotide further includes a poly-A region or a Kozak sequence (e.g., in the 5′-UTR). In some cases, polynucleotides may contain one or more intronic nucleotide sequences capable of being excised from the polynucleotide. In some embodiments, a polynucleotide (e.g., an mRNA) may include a 5′cap structure, a chain terminating nucleotide, a stem loop, a polyA sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl pseudouridine or 1-ethyl-pseudouridine), and/or a 5-substituted cytidine (e.g., 5-methyl-cytidine). In some embodiments, a polynucleotide contains only naturally occurring nucleosides.

In some cases, a polynucleotide is greater than 30 nucleotides in length. In another embodiment, the poly nucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, a polynucleotide molecule, formula, composition or method associated therewith comprises one or more polynucleotides comprising features as described in WO2002/098443, WO2003/051401, WO2008/052770, WO2009/127230, WO2006/122828, WO2008/083949, WO2010/088927, WO2010/037539, WO2004/004743, WO2005/016376, WO2006/024518, WO2007/095,976, WO2008/014979, WO2008/077592, WO2009/030481, WO2009/095226, WO2011/069586, WO2011/026641, WO2011/144358, WO2012/019780, WO2012/013326, WO2012/089338, WO2012/113513, WO2012/116811, WO2012/116810, WO2013/113502, WO2013/113501, WO2013/113736, WO2013/143698, WO2013/143699, WO2013/143700, WO2013/120626, WO2013/120627, WO2013/120628, WO2013/120629, WO2013/174409, WO2014/127917, WO2015/024669, WO2015/024668, WO2015/024667, WO2015/024665, WO2015/024666, WO2015/024664, WO2015/101415, WO2015/101414, WO2015/024667, WO2015/062738, WO2015/101416, all of which are incorporated by reference herein.

Polynucleotides, such as circular RNA, may contain an internal ribosome entry site (IRES). An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picornaviruses (e.g., FMDV), pest viruses (CFFV), polio viruses (PV), encephalomyocarditis viruses (ECMV), foot- and mouth disease viruses (FMDV), hepatitis C viruses (HCV), classical Swine fever viruses (CSFV), murine leukemia virus (MLV), simian immune deficiency viruses (SIV) or cricket paralysis viruses (CrPV).

In some embodiments, a polynucleotide comprises one or more microRNA binding sites. In some embodiments, a microRNA binding site is recognized by a microRNA in a non-target organ. In some embodiments, a microRNA binding site is recognized by a microRNA in the liver. In some embodiments, a microRNA binding site is recognized by a microRNA in hepatic cells.

viii. Additional Lipid Components

The LNP compositions of the present disclosure can further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15(4): 313-320.; Dillard, et al. PNAS 2021 Vol. 118 No. 52.).

### C. Methods and Pharmaceutical Compositions

i. Methods

The pharmaceutical composition may be delivered as described in A.U. Patent Application No. 2017279733, which is incorporated herein by reference in its entirety.

The present disclosure provides methods comprising administering a pharmaceutical composition to a subject in need thereof. The pharmaceutical composition may be administered to a subject using any amount and any route of administration which may be effective for preventing, treating, diagnosing, or imaging a disease, disorder, and/or condition. The exact amount required will vary from subject to subject, depending on factors such as, but not limited to, the species, age, and general condition of the subject, the severity of the disease, the particular composition, its mode of administration, its mode of activity, and the like. The pharmaceutical composition may be administered to animals, such as mammals (e.g., humans, domesticated animals, cats, dogs, monkeys, mice, rats, etc.). The payload of the pharmaceutical composition is a polynucleotide.

In some embodiments, pharmaceutical, prophylactic, diagnostic, or imaging compositions thereof are administered to humans.

In some embodiments, the polynucleotide is administered by one or more of a variety of routes, including, but not limited to, local, oral, intravenous, intramuscular, intra-arterial, intramedullary, intrathecal, subcutaneous, intraventricular, transdermal, interdermal, rectal, intravaginal, intraperitoneal, topical (e.g., by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter.

In some embodiments, polynucleotides are administered by systemic intravenous injection.

In some embodiments, polynucleotides may be administered intravenously and/or orally.

In specific embodiments, polynucleotides may be administered in a way which allows the polynucleotides to cross the blood-brain barrier, vascular barrier, or other epithelial barrier.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Dosage forms for local, topical and/or transdermal administration of a pharmaceutical composition may include ointments, pastes, creams, lotions, gels, powders, solutions, sprays, inhalants and/or patches. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this disclosure.

In general, the most appropriate route of administration will depend upon a variety of factors including the nature of the polynucleotides to be delivered (e.g., its stability in the environment of the gastrointestinal tract, bloodstream, etc), the condition of the patient (e.g., whether the patient is able to tolerate particular routes of administration), etc. The present disclosure encompasses the delivery of the polynucleotide by any appropriate route taking into consideration likely advances in the sciences of drug delivery.

In certain embodiments, pharmaceutical compositions in accordance with the present disclosure may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administration is employed, split dosing regimens such as those described herein may be used.

According to the present disclosure, administration of polynucleotides in split-dose regimens may produce higher levels of proteins in mammalian subjects. As used herein, a “split dose” is the division of single unit dose or total daily dose into two or more doses. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the polynucleotides of the present disclosure are administered to a subject in split doses. The polynucleotides may be formulated in buffer only or in a formulation described herein.

Polynucleotides may be used or administered in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Pharmaceutical compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single pharmaceutical composition or administered separately in different pharmaceutical compositions. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens described herein.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a pharmaceutical composition useful for treating cancer in accordance with the present disclosure may be administered concurrently with a chemotherapeutic agent), or they may achieve different effects (e.g., control of any adverse effects).

Pharmaceutical compositions containing polynucleotides are formulated for administration intramuscularly, transarterially, intraocularly, vaginally, rectally, intraperitoneally, intravenously, intranasally, subcutaneously, endoscopically, transdermally, intramuscularly, intraventricularly, intradermally, intrathecally, topically (e.g. by powders, ointments, creams, gels, lotions, and/or drops), mucosally, nasal, enterally, intratumorally, by intratracheal instillation, bronchial instillation, and/or inhalation; nasal spray and/or aerosol, and/or through a portal vein catheter.

The pharmaceutical compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the pharmaceutical compositions, and the like. In some embodiments, the pharmaceutical composition is formulated for extended release. In specific embodiments, polynucleotides and/or pharmaceutical, prophylactic, diagnostic, or imaging compositions thereof, may be administered in a way which allows the polynucleotides to cross the blood-brain barrier, vascular barrier, or other epithelial barrier.

In some aspects of the present disclosure, the polynucleotides (particularly RNA encoding polynucleotides) are spatially retained within or proximal to a target tissue. Provided are methods of providing a pharmaceutical composition to a target tissue of a mammalian subject by contacting the target tissue (which contains one or more target cells) with the pharmaceutical composition under conditions such that the pharmaceutical composition, in particular the polynucleotide component(s) of the pharmaceutical composition, is substantially retained in the target tissue, meaning that at least 10, 20, 30, 40, 50, 60, 70, 80, 85, 90, 95, 96, 97, 98, 99, 99.9, 99.99 or greater than 99.99% of the pharmaceutical composition is retained in the target tissue. Advantageously, retention is determined by measuring the amount of the polynucleotides present in the pharmaceutical composition that enters one or more target cells. For example, at least 1, 5, 10, 20, 30, 40, 50, 60, 70, 80, 85, 90, 95, 96, 97, 98, 99, 99.9, 99.99 or greater than 99.99% of the polynucleotides administered to the subject are present intracellularly at a period of time following administration. For example, intramuscular injection to a mammalian subject is performed using an aqueous pharmaceutical composition containing an RNA and a transfection reagent, and retention of the pharmaceutical composition is determined by measuring the amount of the RNA present in the muscle cells.

Aspects of the present disclosure are directed to methods of providing a pharmaceutical composition to a target tissue or organ of a mammalian subject, by contacting the target tissue (containing one or more target cells) or organ (containing one or more target cells) with the pharmaceutical composition under conditions such that the pharmaceutical composition is substantially retained in the target tissue or organ. The pharmaceutical composition contains an effective amount of a polynucleotide engineered to avoid an innate immune response of a cell into which the polynucleotide enters. The pharmaceutical compositions generally contain a cell penetration agent, although “naked” polynucleotide (such as polynucleotides without a cell penetration agent or other agent) is also contemplated, and a pharmaceutically acceptable carrier.

Pharmaceutical compositions which may be administered intramuscularly and/or subcutaneously may include, but are not limited to, polymers, copolymers, and gels. The polymers, copolymers and/or gels may further be adjusted to modify release kinetics by adjusting factors such as, but not limited to, molecular weight, particle size, payload and/or ratio of the monomers. As a nonlimiting example, formulations administered intramuscularly and/or subcutaneously may include a copolymer such as poly(lactic-co-glycolic acid).

Localized delivery of the pharmaceutical compositions described herein may be administered by methods such as, but not limited to, topical delivery, ocular delivery, transdermal delivery, and the like. The pharmaceutical composition may also be administered locally to a part of the body not normally available for localized delivery such as, but not limited to, when a subject's body is open to the environment during treatment. The pharmaceutical composition may further be delivered by bathing, soaking and/or surrounding the body part with the pharmaceutical composition.

However, the present disclosure encompasses the delivery of polynucleotides, and/or pharmaceutical, prophylactic, diagnostic, or imaging compositions thereof, by any appropriate route taking into consideration likely advances in the sciences of drug delivery.

ii. Pharmaceutical Compositions

In some embodiments, a nanoparticle includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol. The amount of polynucleotide in a nanoparticle composition may depend on the size, composition, desired target and/or application, or other properties of the nanoparticle composition as well as on the properties of the polynucleotide. For example, the amount of an RNA useful in a nanoparticle composition may depend on the size, sequence, and other characteristics of the RNA. The relative amounts of a polynucleotide and other elements (e.g., lipids) in a nanoparticle composition may also vary. In some embodiments, the wt/wt ratio of the lipid component to a polynucleotide in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a polynucleotide in a nanoparticle composition may, for example, be measured using absorption spectroscopy (e.g., ultraviolet-visible spectroscopy).

In some embodiments, a nanoparticle composition containing a polynucleotide may be formulated to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N.P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N.P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

The mean size of a nanoparticle composition may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload (such as a polynucleotide) describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%. 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21 Edition, A. R. Gennaro: Lippincott, Williams & Wilkins, Baltimore, Md., 2006).

iii. Extrahepatic Delivery

Without intending to be limited to any particular theory, the present disclosure relates to the unexpected discovery of novel lipids and lipid nanoparticle compositions that display specific organ tropism upon administration to a subject, thereby allowing for targeted delivery of therapeutic polynucleotide payloads.

In one aspect, the present disclosure provides LNP formulations that enable extrahepatic delivery of a polynucleotide payload, and methods using the same. As used herein, the term “substantial extrahepatic delivery” is meant to indicate that the LNP formulations enable delivery of a polynucleotide payload to organs outside of the liver in a subject, preferably a mammalian subject, upon administration. In some embodiments, “substantial” extrahepatic delivery means delivery of at least 5% of the polynucleotide payload to organs outside of the liver. In some aspects, the present disclosure provides LNP formulations that enable delivery a polynucleotide payload to a target organ that is not the liver of a subject. In some embodiments, the target organ is the kidney, placenta, heart, lung, muscle, fat, bladder, spleen, adrenal glands, brain, vagina, immune system, central nervous system, or skin of a subject.

In some embodiments, the present disclosure provides methods of treating a disease or disorder using an LNP of the present disclosure wherein extrahepatic delivery of the polynucleotide payload is beneficial or desirable. In some embodiments, the present disclosure provides methods of treating a disease or disorder using an LNP of the present disclosure wherein delivery of the polynucleotide payload to a specific target organ is beneficial or desirable.

In some embodiments, the disease or disorder is one that affects, or is related to the kidneys of a subject. In some embodiments, the disease or disorder is one that affects, or is related to the placenta of a subject. In some embodiments, the disease or disorder is one that affects, or is related to the heart of a subject. In some embodiments, the disease or disorder is one that affects, or is related to the lungs of a subject. In some embodiments, the disease or disorder is one that affects, or is related to the muscle of a subject. In some embodiments, the disease or disorder is one that affects, or is related to the fat of a subject. In some embodiments, the disease or disorder is one that affects, or is related to the bladder of a subject. In some embodiments, the disease or disorder is one that affects, or is related to the spleen of a subject. In some embodiments, the disease or disorder is one that affects, or is related to the adrenal glands of a subject. In some embodiments, the disease or disorder is one that affects, or is related to the brain of a subject. In some embodiments, the disease or disorder is one that affects, or is related to the central nervous system of a subject. In some embodiments, the disease or disorder is one that affects, or is related to the skin of a subject. In some embodiments, the disease or disorder is one that affects, or is related to the vagina of a subject. In some embodiments, the disease or disorder is one that affects, or is related to the immune system of a subject.

In some embodiments, the LNP formulations enable about 5%, 10%, 15%, 20%, 25%, 30%, 40%, 45%, 50%, 55%, 60%, 65%, 70%, 75%, 80%, 85%, 90%, or 95% of the polynucleotide payload delivery to occur in a target organ, as compared to delivery to non-target organs, when administered to a mammalian subject. In some embodiments, the lipid nanoparticle delivers a higher proportion of its payload to a target organ than to the liver. In some embodiments, the lipid nanoparticle delivers a proportionately higher proportion of its payload to a target organ than to the liver, after accounting for the weight of the target organ and the liver. For example, one can measure the amount of payload delivered to the target organ and liver and divide the measured amount of payload delivered to the organ by the weight of the respective organ. In some embodiments, the lipid nanoparticle delivers more of its payload to a target organ than a reference lipid nanoparticle does. In some embodiments, the reference lipid nanoparticle is one disclosed in Angew. Chem. Int. Ed. 2020, 59, 20083-20089 or Lokugamage, et al. Adv. Mater. 2019, 1902251. In some embodiments, the reference lipid nanoparticle comprises at least one ionizable lipid selected from Compound 3 and Compound 4.

In some embodiments, the preferential delivery of a payload to a target organ by the LNP formulations of the present disclosure allows for administration of a lower dosage of the LNP to be administered to the subject than would be needed for a reference LNP formulation. In some embodiments, the preferential delivery of a payload to a target organ by the LNP formulations of the present disclosure yields a greater therapeutic effect when administered to a subject than a reference LNP formulation administered at the same dosage.

### D. Particular Embodiments

The present disclosure provides the following particular embodiments.

Embodiment 1. A pharmaceutical composition formulated for substantial extrahepatic delivery comprising:
a. a lipid nanoparticle comprising an ionizable lipid; and
b. a polynucleotide.

Embodiment 2. The pharmaceutical composition of Embodiment 1, wherein at least about 5%, 10%, 15%, 20%, 25%, 30%, 40%, 45%, 50%, 55%, 60%, 65%, 70%, 75%, 80%, 85%, 90%, or 95% of payload delivery occurs in a target organ.

Embodiment 3. The pharmaceutical composition of any preceding Embodiment, wherein the lipid nanoparticle delivers a higher proportion of its payload to a target organ than to the liver.

Embodiment 4. The pharmaceutical composition of any preceding Embodiment, wherein the lipid nanoparticle delivers more of its payload to a target organ than a reference lipid nanoparticle does.

Embodiment 5. The pharmaceutical composition of Embodiment 4, wherein the reference lipid nanoparticle comprises MC3.

Embodiment 6. The pharmaceutical composition of Embodiment 4, wherein the lipid portion of the reference lipid nanoparticle comprises about 50 mol % MC3, about 10 mol % DSPC, about 38.5 mol % cholesterol, and about 1.5 mol % PEG-DMG.

Embodiment 7. The pharmaceutical composition of any preceding Embodiment, wherein the ionizable lipid has a structure according to any of formulas 1-6.

Embodiment 8. The pharmaceutical composition of any preceding Embodiment, wherein the ionizable lipid has a head group listed on Table 1.

Embodiment 9. The pharmaceutical composition of any preceding Embodiment, wherein the ionizable lipid has a head group that contains a short peptide of 12-15 mer length.

Embodiment 10. The pharmaceutical composition of any preceding Embodiment, wherein the ionizable lipid has a head group that contains the structure of Vitamin A, D, E, or K.

Embodiment 11. The pharmaceutical composition of any preceding Embodiment, wherein the ionizable lipid has an alkyl tail.

Embodiment 12. The pharmaceutical composition of any preceding Embodiment, wherein the ionizable lipid has a disulfide tail.

Embodiment 13. The pharmaceutical composition of any preceding Embodiment, wherein the ionizable lipid contains an ester.

Embodiment 14. The pharmaceutical composition of any preceding Embodiment, wherein the ionizable lipid contains 1, 2, 3, or more branches.

Embodiment 15. The pharmaceutical composition of any preceding Embodiment, wherein the ionizable lipid has asymmetrical tails.

Embodiment 16. The pharmaceutical composition of any preceding Embodiment, wherein the ionizable lipid has a pKa between 6 and 7.

Embodiment 17. The pharmaceutical composition of any preceding Embodiment, wherein the ionizable lipid is positively charged.

Embodiment 18. The pharmaceutical composition of Embodiment 16 or 17, wherein the target organ is the lung.

Embodiment 19. The pharmaceutical composition of any preceding Embodiment, wherein the lipid nanoparticle further comprises a PEGylated lipid.

Embodiment 20. The pharmaceutical composition of Embodiment 19, wherein the PEGylated lipid is PEG-DMG.

Embodiment 21. The pharmaceutical composition of any preceding Embodiment, wherein the lipid nanoparticle further comprises a structural lipid.

Embodiment 22. The pharmaceutical composition of Embodiment 21, wherein the structural lipid is cholesterol.

Embodiment 23. The pharmaceutical composition of Embodiment 21, wherein the structural lipid is a cholesterol analog.

Embodiment 24. The pharmaceutical composition of Embodiment 21, wherein the structural lipid contains a plant sterol mimetic.

Embodiment 25. The pharmaceutical composition of any preceding Embodiment, wherein the lipid nanoparticle further comprises a phospholipid.

Embodiment 26. The pharmaceutical composition of Embodiment 25, wherein the phospholipid is modified for enhanced endosomal escape.

Embodiment 27. The pharmaceutical composition of Embodiment 25, wherein the phospholipid is selected from DOPE and DSPC.

Embodiment 28. The pharmaceutical composition of any one of Embodiments 1-27, wherein the polynucleotide is DNA.

Embodiment 29. The pharmaceutical composition of any one of Embodiments 1-27, wherein the polynucleotide is RNA.

Embodiment 30. The pharmaceutical composition of Embodiment 29, wherein the RNA is circular RNA.

Embodiment 31. The pharmaceutical composition of Embodiment 29, wherein the RNA is a short interfering RNA (siRNA), an asymmetrical interfering RNA (aiRNA), a microRNA (miRNA), a Dicer-substrate RNA (dsRNA), or a short hairpin RNA (shRNA).

Embodiment 32. The pharmaceutical composition of Embodiment 31, wherein the RNA consists of fewer than about 15, 20, 25, 30, or 50 nucleotides.

Embodiment 33. The pharmaceutical composition of any one of Embodiments 28-30, wherein the polynucleotide encodes a protein.

Embodiment 34. The pharmaceutical composition of Embodiment 33, wherein the polynucleotide comprises at least about 15, 20, 25, 30, 50, 100, 500, 1000, 1500, 2000, 2500, 3000, 4000, 5000, 6000, 7000, 8000, 9000, 10000, or greater than 10000 nucleotides.

Embodiment 35. The pharmaceutical composition of any preceding Embodiment, wherein the polynucleotide has been modified by a glycan.

Embodiment 36. The pharmaceutical composition of any preceding Embodiment, wherein the polynucleotide consists of natural nucleotides.

Embodiment 37. The pharmaceutical composition of any one of Embodiments 1-36, formulated for systemic administration to a human subject in need thereof.

Embodiment 38. The pharmaceutical composition of any one of Embodiments 1-36, formulated for administration into a target organ in a human subject in need thereof.

Embodiment 39. The pharmaceutical composition of any preceding Embodiment, wherein the target organ is the kidney, placenta, heart, lung, muscle, fat, bladder, spleen, adrenal glands, brain, vagina, immune system, central nervous system, or skin.

Embodiment 40. The pharmaceutical composition of any preceding Embodiment, wherein at least about 5%, 10%, 15%, 20%, 25%, 30%, 40%, 45%, 50%, 55%, 60%, 65%, 70%, 75%, 80%, 85%, 90%, or 95% of polynucleotides are encapsulated within lipid nanoparticles.

Embodiment 41. The pharmaceutical composition of any preceding Embodiment, further comprising a target organ binding moiety.

Embodiment 42. The pharmaceutical composition of Embodiment 41, wherein the target organ binding moiety is operably connected to the lipid nanoparticle.

Embodiment 43. A method of treating a subject in need thereof, comprising administering to the subject the pharmaceutical composition of any preceding Embodiment.

Embodiment 44. A method of treating a subject in need thereof, comprising administering to the subject the pharmaceutical composition of any preceding Embodiment through system administration.

Embodiment 45. A method of treating a subject in need thereof, comprising administering to the subject the pharmaceutical composition of any preceding Embodiment through local administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

### EXAMPLES

The following are examples of methods and compositions of the present disclosure. It is understood that various other embodiments may be practiced, given the general description provided herein.

### Example 1: Production of Nanoparticle Compositions

A nanoparticle composition may be produced as described in US patent application US20170210697A1, which is incorporated herein by reference in its entirety.

In order to investigate safe and efficacious nanoparticle compositions for use in the delivery of polynucleotides to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the polynucleotide and the other has the lipid components.

Lipid compositions are prepared by combining an ionizable lipid, a phospholipid (such as DOPE or DSPC, obtainable from Avanti Polar Lipids, Alabaster, Ala.), a PEG lipid (such as PEG-DMG, obtainable from Avanti Polar Lipids, Alabaster, Ala.), and a structural lipid (such as cholesterol, obtainable from Sigma-Aldrich, Taufkirchen, Germany, or a cholesterol analog) in ethanol. Lipids are combined to yield desired molar ratios and diluted with water and ethanol.

Nanoparticle compositions may be prepared by combining a lipid solution with a solution including the polynucleotide component. The lipid solution is rapidly injected using, for example, a NanoAssemblr microfluidic based system, into the polynucleotide solution.

Solutions of the polynucleotide in deionized water may be diluted in citrate buffer to form a stock solution.

Nanoparticle compositions can be processed by dialysis to remove ethanol and achieve buffer exchange. Formulations are dialyzed against a buffer such as phosphate buffered saline (PBS), Tris-HCl, or sodium citrate, using, for example, Slide-A-Lyzer cassettes (Thermo Fisher Scientific Inc., Rockford, Ill.). The resulting nanoparticle suspension is filtered through sterile filters (Sarstedt, Nümbrecht, Germany) into glass vials and sealed with crimp closures. Alternatively, a Tangential Flow Filtration (TFF) system, such as a Spectrum KrosFlo system, may be used.

The method described above induces nano-precipitation and particle formation. Alternative processes including, but not limited to, T-junction and direct injection, may be used to achieve the same nano-precipitation.

### Example 2: Characterization of Nanoparticle Compositions

A nanoparticle composition may be characterized as described in US patent application US20170210697A1, which is incorporated herein by reference in its entirety.

Particle size, polydispersity index (PDI), and the zeta potential of a nanoparticle composition can be determined using, for example, a ZetasizerNano ZS (Malvern Instruments Ltd, Malvern, Worcestershire, UK), or a Wyatt DynaPro plate reader.

Ultraviolet-visible spectroscopy can be used to determine the concentration of a polynucleotide in nanoparticle compositions. The formulation may be diluted in PBS then added to a mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of polynucleotides in the nanoparticle composition can be calculated based on the extinction coefficient of the polynucleotide used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted in a TE buffer solution. Portions of the diluted samples are transferred to a polystyrene 96 well plate and either TE buffer or a 2% Triton X-100 solution is added to the wells. The plate is incubated at, for example, a temperature of 37° C. for 15 minutes. The RIBOGREEN® reagent is diluted in TE buffer, and this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### Example 3: In Vivo Studies Including Protein Expression by Organ

Delivery to a target organ may be assessed as described in US patent application US20170210697A1, which is incorporated herein by reference in its entirety.

In order to monitor how effectively various nanoparticle compositions deliver polynucleotides to targeted cells, different nanoparticle compositions including a particular polynucleotide are prepared and administered to rodent populations. Mice are intravenously, intramuscularly, intraarterially, or intratumorally administered a single dose of a nanoparticle composition. In some instances, mice may be made to inhale doses. Dose sizes may range from 0.001 mg/kg to 10 mg/kg, where 10 mg/kg describes a dose including 10 mg of polynucleotide in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. Time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

For example, LNP formulations including RNA encoding a detectable protein such as luciferase may be administered intravenously to mice at a dosage of, for example, 0.5 mg/kg. A standard MC3 formulation and a PBS control may also be tested. Bioluminescence in various organs, such as the liver, lung, spleen, and femur, may be measured after 6 hours.

Nanoparticle compositions including protein coding RNA are useful in the evaluation of the efficacy and usefulness of various formulations for the delivery of polynucleotides. Higher levels of protein expression induced by administration of a composition including protein coding RNA will be indicative of higher RNA translation and/or nanoparticle composition RNA delivery efficiencies. As the non-RNA components are not thought to affect translational machineries themselves, a higher level of protein expression is likely indicative of a higher efficiency of delivery of the RNA by a given nanoparticle composition relative to other nanoparticle compositions or the absence thereof.

### Example 4: Toxicity, Cytokine Induction, and Complement Activation

Toxicity may be analyzed as described by international patent application WO2016118724 and/or US20170210697A1, which are incorporated herein by reference in its entirety.

### Example 4a: Liver Toxicity

RNA encoding a detectable protein is generated and loaded into lipid nanoparticles. The nanoparticles are administered to mice, and expression of the detectable protein as well as levels of certain liver enzymes are measured. Additional mice may be dosed with a reference LNP formulation, such as one containing MC3, as a comparison. To assess dose response, mice may be given varying levels of the LNP formulations. Liver enzymes, such as alanine transaminase (ALT) and aspartate transaminase (AST), may be measured to assess liver toxicity. In some embodiments, creatine phosphokinase (CPK) may also be measured to assess cardiac or muscular toxicity. In some embodiments, a pharmaceutical composition described herein provides a safer toxicity profile than a reference pharmaceutical composition, such as one containing MC3.

### Example 4b: Cytokine Induction

The introduction of foreign material into a mammalian body induces an innate immune response that promotes cytokine production. Such immune responses to, for example, nanoparticle compositions including polynucleotides, are undesirable. The induction of certain cytokines is thus measured to evaluate the efficacy of nanoparticle compositions and the inflammatory response. The concentrations of various cytokines in mice upon intravenous administration of nanoparticle compositions at a dosage of 0.5 mg/kg are measured at 6 hours. The standard MC3 formulation and a PBS control may also be tested. Cytokines including TNF-a, IFN-γ, IP-10, MCP-1, IFN-a, IL-6, and IL-5 may be measured. In some embodiments, IP-10 and IL-6 are measured. In some embodiments, histamine levels may also be measured. In some embodiments, a pharmaceutical composition described herein provides an improved inflammatory profile than a reference pharmaceutical composition, such as one containing MC3.

### Example 4c: Complement Activation

Complement activation assists in the clearance of pathogens from an organism. As it is undesirable that a subject's body recognize a nanoparticle composition as a foreign invader, low complement system activation upon administration of such a composition is preferred. The complex sC5b-9 is a marker for the activation of the complement system. Thus, human cells are contacted in vitro with nanoparticle compositions and are evaluated for sC5b-9 levels.

### Example 5: LNP Optimization

LNP compositions may be optimized as described by US patent application US20170210697A1, which is incorporated herein by reference in its entirety.

### Example 5a: Optimization of Lipid: Polynucleotide Ratios

The relative amounts of lipid component and polynucleotide in a nanoparticle composition can be optimized according to considerations of efficacy and tolerability.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition. Thus, the wt/wt ratio of total lipid to RNA is varied between, for example, 10:1, 15:1, 20:1, 32:1, 40:1, 50:1, and 60:1 for a lipid formulation including about 50 mol % ionizable lipid, about 10 mol % phospholipid (e.g., DOPE or DSPC), about 38.5 mol % structural lipid (e.g., cholesterol), and about 1.5 mol % PEG lipid (e.g., PEG-DMG or PEG-DSPE). N:P ratios are calculated for each nanoparticle composition assuming a single protonated nitrogen atom. The encapsulation efficiency (EE), size, and polydispersity index of each composition are also measured.

Generally, compositions with higher total lipid:RNA ratios yield smaller particles with higher encapsulation efficiencies, both of which are desirable. However, the N:P ratio for such formulations generally exceeds 4. Current standards in the art such as the MC3 formulation described above have N:P ratios of 5.67. Thus, a balance between the N:P ratio, size, and encapsulation efficiency should be struck. Ratios may be optimized such that the N:P ratio is less than, for example, 5 or 6.

In order to explore the efficacy of nanoparticle compositions with different N:P ratios, the expression of a detectable protein such as luciferase (Luc) or human erythropoietin (hEPO) in mice after low (0.05 mg/kg) or high (0.5 mg/kg) doses of intravenously administered nanoparticle compositions is examined. The concentration of Luc or hEPO expressed is measured 3, 6, and/or 24 hours after administration.

### Example 5b: Optimization of Ionizable Lipid

As smaller particles with higher encapsulation efficiencies are generally desirable, the relative amounts of various elements in lipid components of nanoparticle compositions are optimized according to these parameters.

An ionizable lipid is selected for optimization. The relative amount of the ionizable lipid is varied between 30 mol % and 60 mol % in compositions including DOPE or DSPC as phospholipids to determine the optimal amount of the ionizable lipid in the formulations. Formulations are prepared using a standardized process with a water to ethanol ratio in the lipid-mRNA solution of, for example, 3:1 and a rate of injection of the lipid solution into the mRNA solution of, for example, 12 mL/min on a NanoAssemblr microfluidic based system. These parameters may be altered depending on, for example, the lipids used and the target particle size. This method induces nano-precipitation and particle formation. Alternative processes including, but not limited to, T-junction or direct injection, may also be used to achieve the same nano-precipitation.

Formulations producing the smallest particles with the highest encapsulation efficiencies are generally preferred, however larger or smaller particle sizes may be desirable based on a given application (e.g., based on the fenestration size of a target organ). Compositions are also evaluated for their detectable protein expression levels and cytokine profiles.

### Example 5c: Optimization of Phospholipid

The relative amount of phospholipid in a lipid component of a nanoparticle composition is varied to further optimize the formulation. An ionizable lipid is selected for use in the nanoparticle composition and DOPE and DSPC are selected as phospholipids. Additional phospholipids can also be evaluated. Nanoparticle compositions are prepared with the relative phospholipid content varying between 0 mol % and 30 mol %. Compositions are evaluated for their size, encapsulation efficiency, detectable protein expression levels, and cytokine profiles.

### Example 5d: Optimization of Structural Lipid

The relative amount of structural lipid in a lipid component of a nanoparticle composition is varied to further optimize the formulation. An ionizable lipid is selected for use in the nanoparticle composition and cholesterol or a cholesterol analog is selected as a structural lipid. Additional structural lipids can also be evaluated. Nanoparticle compositions are prepared with the relative structural lipid content varying between 18.5 mol % and 48.5 mol %. Compositions are evaluated for their size, encapsulation efficiency, detectable protein expression levels, and cytokine profiles.

### Example 5e: Optimization of PEG Lipid

The relative amount of PEG lipid in a lipid component of a nanoparticle composition is varied to further optimize the formulation. An ionizable lipid is selected for use in the nanoparticle composition and PEG-DMG or PEG-DSPE is selected as a PEG lipid. Additional PEG lipids can also be evaluated. Nanoparticle compositions are prepared with the relative PEG lipid content varying between 0 mol % and 10 mol %. Compositions are evaluated for their size, encapsulation efficiency, detectable protein expression levels, and cytokine profiles.

### Example 5f: Optimization of Particle Sizes

The fenestration sizes for different bodily organs often vary; for example, the kidney is known to have a smaller fenestration size than the liver. Thus, targeting delivery of a polynucleotide (e.g., specifically delivering) to a particular organ or group of organs may require the administration of nanoparticle compositions with different particle sizes. In order to investigate this effect, nanoparticle compositions are prepared with a variety of particle sizes using a Nanoassemblr instrument. Nanoparticle compositions include an RNA encoding Luc. Each differently sized nanoparticle composition is subsequently administered to mice to evaluate the effect of particle size on delivery selectivity. Luc expression in two or more organs or groups of organs can be measured using bioluminescence to evaluate the relative expression in each organ.

### Example 6: Circular RNA

Circular RNA may be designed and produced as described in international patent application WO2020219563, which is incorporated herein by reference in its entirety.

### Example 6a: Circular RNA Design

Translation of a protein such as GFP from circular RNA is achieved by using a ribozyme in a permuted intron-exon (PIE) splicing strategy. To create a circRNA encoding GFPs, an internal ribosome entry site (IRES), following by a coding sequence, are placed between two short fragments of the E2 and E1 exons downstream and upstream of the group I catalytic intron in the thymidylate synthase (Td) gene from phage T4. Alternatively, E2 and E1 exons downstream and upstream of the group I catalytic intron in Anabaena pre-tRNA gene can be used as splicing efficiency of group I catalytic intron is more efficient in Anabaena pre-tRNA gene than in phage T4 Td gene. [Puttaraju, M. & Been, M. Nucleic Acids Res. 20, 5357-5364 (1992)]. Finally, the 3′ half of the group I catalytic intron is cloned upstream of E2 whereas the 5′ half of the group I catalytic intron is placed downstream of E1. A spacer between the 3′ PIE splice site and the IRES are designed. Complementary ‘homology arms’ are placed at the 5′ and 3′ ends of the precursor RNA with the aim of bringing the 5′ and 3′ splice sites into proximity of one another are used during the circulation process to increase splicing efficiency. In some embodiments, the homology arms may be between 33-35 nucleotides in length.

### Example 6b: Precursor RNA Production

A protein coding locus, Anabaena catalytic intron, and an IRES (such as a Coxsackievirus B3 (CVB3) or encephalomyocarditis virus (EMCV) IRES) sequence are synthesized. The sequences are subsequently cloned into a linearized plasmid vector containing a T7 RNA polymerase promoter by Gibson Assembly® such as by using a NEBuilder® HiFi DNA Assembly kit (New England Biolabs). Spacer regions, homology arms, and other variations are introduced using a Q5® Site-Directed Mutagenesis Kit (New England Biolabs). Linear precursor RNAs are synthesized by in vitro transcription from a linearized plasmid DNA template or PCR product using a T7 High Yield RNA Synthesis Kit (New England Biolabs).

### Example 6c: Production and Purification of Protein Coding circRNA

Linear precursor RNA is treated with DNase I (New England Biolabs) for 20 min after in vitro transcription. The RNA samples are then column purified using a MEGAclear Transcription Clean up kit (Ambion). Linear precursor RNAs are then heated in the presence of magnesium ions and GTP to promote circularization, essentially as described previously for the circularization of shorter RNAs [Ford, E. & Ares, M. Proc. Natl Acad. Sci. 91, 3117-3121 (1994)]: RNA is heated to 70 C for 5 min and then immediately placed on ice for 3 min, after which GTP is added to a final concentration of 2 mM along with a buffer including magnesium (50 mM Tris-HCl, 10 mM MgCl2, 1 mM DTT, pH 7.5; New England Biolabs). RNA is then heated to 55 C for 40 min and then column purified.

Circularity check of the RNA using RNase R: To enrich for circRNA, 20 pg of RNA are diluted in water (88 pL final volume) and then heated at 70C for 2 min and cooled on ice for 2 min. 20 U RNase R and 10 pL of 10A˜RNase R buffer (Epicenter) are added, and the reaction is incubated at 37C for 40 min; an additional 10 U RNase R are added halfway through the reaction. RNase R-digested RNA is column purified using Monarch4 RN A Cleanup Kit (New England Bioiabs).

RNA is separated on precast 1.5% TBE agarose gel or precast 2% E-gel EX agarose gels (Invitrogen); ssRNA Ladders (NEB, ThermoFisher Scientific) is used as a standard. Bands are visualized using blue light transillumination. For gel extractions, bands corresponding to the circRNA are excised from the gel and then extracted using a Zymoclean™ Gel RNA Extraction Kit (Zymogen).

Purity of circRNA preparations is another factor essential for maximizing protein production from circRNA and for avoiding innate cellular immune responses [Kariko, K., Muramatsu, H., Ludwig, J. & Weissman, D. Nucleic Acids Res. 39, e142-e142 (2011)]. Therefore, as an alternative column purification method high-performance liquid chromatography (HPLC), fast protein liquid chromatography (FPLC), or size exclusion chromatography is applied. For HPLC 30 pg of RNA is heated at 65° C. for 3 min and then placed on ice for 3 min. RNA is run through a 4.6 c 300 mm size-exclusion column with a particle size of 5 pm and a pore size of 200 A (Sepax Technologies; part number: 215980P-4630) on an Agilent 1100 Series HPLC (Agilent). RNA is rim in Rnase-free TE buffer (10 mM Tris, 1 mM EDTA, pH: 6) at a flow rate of 0.3 mL/minute. RNA is detected by UV absorbance at 260 nm, but is collected without UV detection. Resulting RNA fractions are precipitated with 5 M ammonium acetate, resuspended in water, and then in some cases treated with Rnase R as described above.

RNAs are purified from crude transcription reactions using an AKTA prime FPLC system equipped with a 50 mL superloop and three 5 mL HiTrap DEAE-sepharose FF columns (GE Healthcare) connected in series. The DEAE columns are equilibrated with three column volumes of buffer A (50 mM sodium phosphate [pH 6.5], 150 mM sodium chloride, and 0.2 mM EDTA) at room temperature. Buffer B contains the same components with 2 M sodium chloride. Both buffers can be prepared in large quantities, sterile filtered, and stored at 4° C. (buffer A) or room temperature (buffer B) to avoid precipitation of sodium chloride. The stopped transcription reaction (10\hat{ }10 mL) is loaded into the 50 mL superloop and weak anion-exchange chromatography is performed using the following gradient, while collecting 10 mL fractions in sterile 15 mL plastic tubes: 0-70 mL (0% B at 1 mL/min) to load the sample onto the DEAE columns, 70-100 mL (0% 10% B at 2 mL/min) to wash remaining rNTPs off the column, 100-380 mL (10%-30% B at 2 mL/min) to separate small abortive transcripts, the desired RNA product, and the plasmid DNA template, 380-410 mL (30%-100% B at 4 mL/min), 410-455 mL (100% B at 4 mL/min), and 455-485 mL (100%-0% B at 4 mL/min) to equilibrate the column for the next purification. Lor small-scale transcriptions below 1 mL, the reaction mixture is diluted to 2 mL with buffer A to ensure complete loading into the superloop and chromatography performed using a single 1-mL HiTrap DEAE-sepharose PL column and the same gradient profile with buffer volumes reduced to 1/15 collecting 2 mL fractions. Fractions containing desired RNA are precipitated with 5 M ammonium acetate, resuspended in water, and in some embodiments treated with RNAse R.

Lor SEC, the AKTA pure system is used connected to a PR-9 fraction collection under control of UNICORN 7.0 software suite. Circular RNA was injected through 0.5 ml sample loop to Superdex 200 increase column (24 ml). The column is equilibrated with PBS (NaCl 0.138M; KC1−0.0027M); pH=7.2 prepared in DEPC treated water. Chromatography is performed at 0.2 mL/min, collecting 0.5 or 0.25 ml fractions. All experiments were performed at 4° C.

### Example 7: Ionizable Lipid Synthesis

### Example 7a: Synthesis of Compound 1

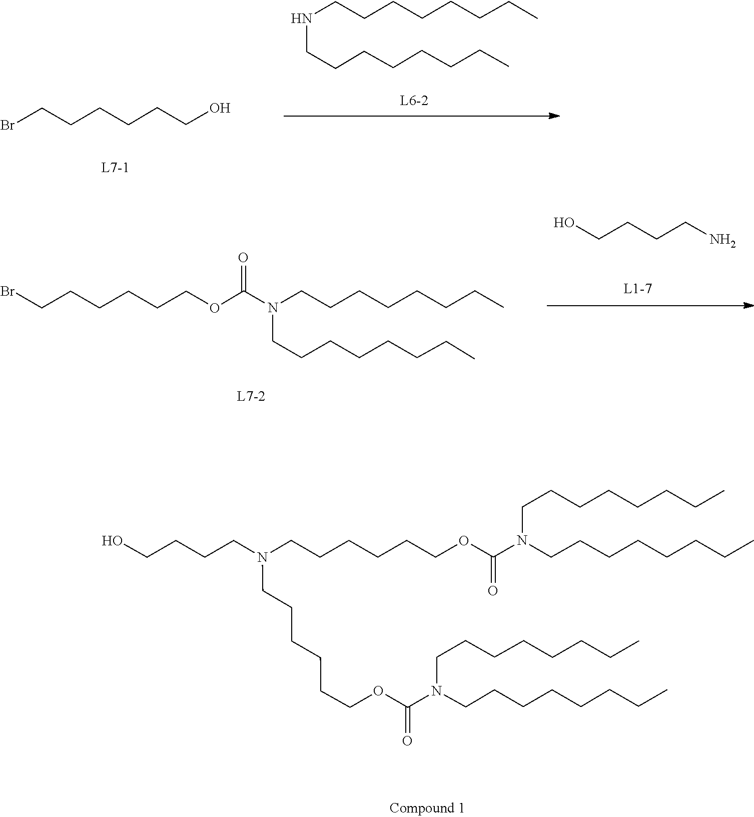 [SMILES: CCCCCCCCNCCCCCCCC ; OCCCCCCBr ; NCCCCO ; CCCCCCCCN(CCCCCCCC)C(=O)OCCCCCCBr ; CCCCCCCCN(CCCCCCCC)C(=O)OCCCCCCN(CCCCO)CCCCCCOC(=O)N(CCCCCCCC)CCCCCCCC]

### Synthesis of 6-bromohexyl dioctylcarbamate (L7-2)

A solution of compound L7-1 (1.0 g, 5.52 mmol) in DCM (20 mL), cooled in an ice-water bath and under nitrogen, was treated with triphosgene (1.63 g, 5.52 mmol) over 5 min followed by the addition of DIPEA (2.8 mL, 16.5 mmol) and stirred for 5 min. The mixture was allowed to room temperature and stirred for 1 h and then compound-L6-2 was added in one portion. The resulting mixture was allowed to warm to room temperature and stirred for 12 h. The solvent was removed in vacuo, and the residue was dissolved in ethyl acetate and washed with saturated NaHCO₃ and brine and dried (Na₂SO₄). Concentration in vacuo to give crude product which was purified by silica gel column chromatography eluted with a gradient of hexane/ethyl acetate to obtain final Compound L7-2 (1.48 g, 60%); ¹H-NMR (300 MHz, CDCl₃) δ 4.04 (t, 2H), 3.39 (t, 2H), 3.16 (s, 4H), 1.90-1.74 (m, 2H), 1.64-1.58 (m, 2H), 1.51-1.37 (m, 7H), 1.36-1.12 (m, 21H), 0.87 (t, 6H). CIMS m/z 449.2 [M+H]⁺.

### Synthesis of ((4-hydroxybutyl) azanediyl) bis(hexane-6,1-diyl) bis(dioctylcarbamate) (Compound 1)

To a solution of compound L1-7 (0.05 g, 0.5 mmol) in CPME (5 mL) and (ACN 5 mL), under nitrogen, was added compound L7-2 (0.503 g, 1.1 mmol) and followed by the addition of K₂CO₃ (0.310 g, 2.2 mmol, 4 eq) and KI (0.093 g, 0.56 mmol). The reaction mixture was heated at 80° C. for 18 h. After cooled to room temperature, the reaction mixture was filtered through Celite, washed with ethyl acetate, and the solvent removed under vacuum to give the crude product which was purified by flash chromatography (SiO₂: DCM/MeOH 0-10%) to give Compound 3 (0.2 g, 45%) as colorless oil; ¹H-NMR (300 MHz, CDCl₃) δ 4.03 (t, 4H), 3.61 (t, 2H), 3.15-3.14 (m, 7H), 2.71-2.69 (m, 5H), 1.78-1.75 (m, 10H), 1.71-1.58 (m, 15H), 1.41-1.26 (m, 46H), 0.84 (t, 12H); CIMS m/z 824.7 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.4 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=13.8 min, purity: 99.38%.

### Example 7b: Synthesis of Compound 2

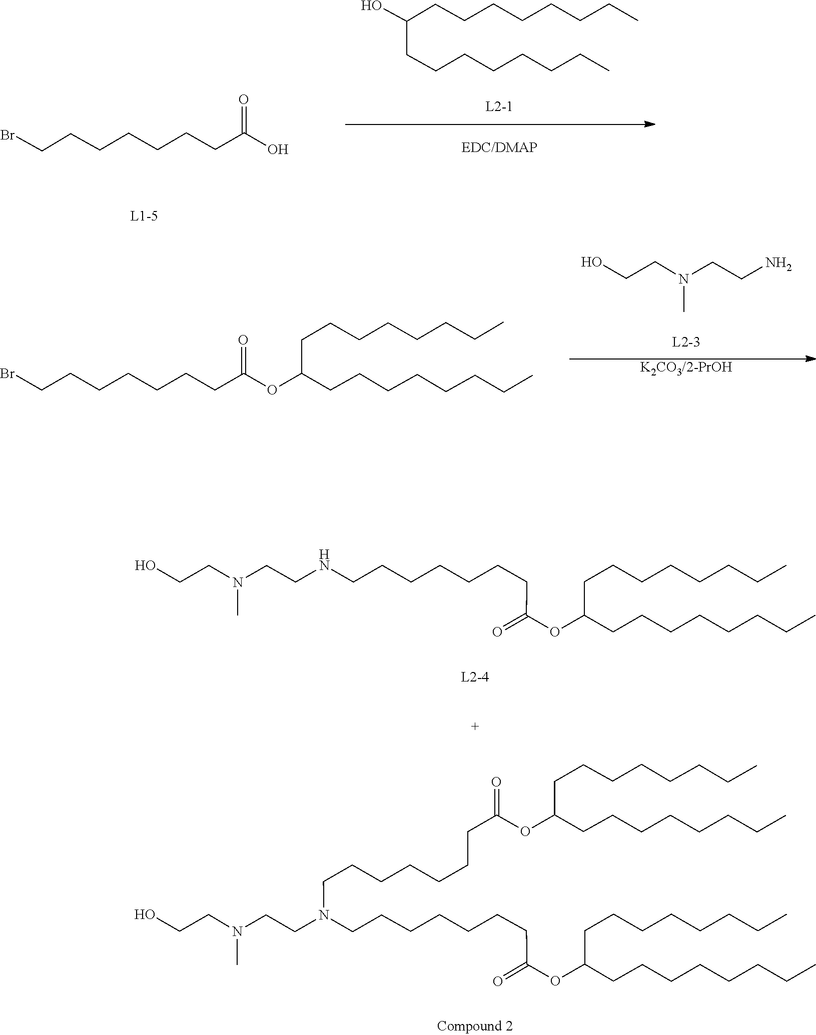

### Synthesis of heptadecan-9-yl 8-bromooctanoate (L2-2)

To a solution of L2-1 (2.56 g, 1 mmol) in DCM (60 mL) was added 8-bromooctanoic acid L1-5 (2.22 g, 1 mmol) followed by DMAP (0.61 g, 0.5 mmol) and EDC (3.9 g, 2 mmol). The resulting mixture was stirred at room temperature under nitrogen atmosphere overnight. The reaction mixture was diluted with DCM (50 mL) and washed with saturated NaHCO₃ aqueous solution (50 mL), water (30 mL) and brine (30 mL). The organic phase was dried over anhydrous Na₂SO₄. Filtration and concentration provided crude material which was purified by flash column chromatography (SiO₂: 0 to 10% ethyl acetate in hexane gradient) to yield 3-pentyloctyl 8-bromooctanoate L2-2 as colorless oil (3.2 g, 70%). ¹H-NMR (300 MHz, CDCl₃) δ 4.85 (quintet, J=6.1 Hz, 1H), 3.40 (t, J=6.9 Hz, 2H), 2.28 (t, J=7.7 Hz, 2H), 1.86 (quintet, J=6.0 Hz, 2H), 1.70-1.15 (m, 36H), 0.87 (t, J=6.9 Hz, 6H).

### Synthesis of heptadecan-9-yl 8-((2-((2-hydroxyethyl)(methyl)amino)ethyl)amino)octanoate (L2-4) and di(heptadecan-9-yl) 8,8′-((2-((2-hydroxyethyl)(methyl)amino)ethyl)azanediyl)dioctanoate (Compound 2)

A mixture of L2-2 (1.0 g, 2.2 mmol), 2-[(2-aminoethyl)(methyl)amino]ethanol L2-3 (1.28 g, 11 mmol) in 2-propanol (10 mL) containing potassium carbonate (0.28 g, 2.0 mmol) was heated at 55-60° C. for 3.5 days. After cooled to room temperature, the reaction mixture was filtered through Celite. Concentration gave an oil residue which was purified by flash chromatography (SiO₂: ethyl acetate/hexane 0-100% with 1-30% triethylamine in the eluent) to give L2-4 (0.79 g, 72%) as slightly yellow oil. ¹H NMR (300 MHz, CDCl₃): δ ppm 4.85 (quintet, J=6.1 Hz, 1H), 3.59 (t, J=5.2 Hz, 2H), 2.70 (t, J=6.1 Hz, 2H), 2.62-2.48 (m, 6H), 2.29 (s, 3H), 2.27 (t, J=7.7 Hz, 2H), 1.73-1.16 (m, 40H), 0.87 (t, J=6.6 Hz, 6H); MS (CI): m/z [M+H]⁺ 499; The bis-addition product Compound 2 (69 mg) was also isolated as slightly yellow oil. ¹H NMR (300 MHz, CDCl₃): δ ppm 4.86 (quintet, J=6.3 Hz, 1H), 3.55 (t, J=4.9 Hz, 2H), 2.54 (t, J=5.4 Hz, 2H), 2.49 (s, 4H), 2.40 (t, J=7.4 Hz, 4H), 2.30 (s, 3H), 2.27 (t, J=7.6 Hz, 4H), 1.73-1.16 (m, 84H), 0.87 (t, J=6.6 Hz, 6H); MS (CI): m/z [M+H]⁺ 879.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=12.0 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=14.8 min, purity: 99.42%.

### Example 7c: Synthesis of Compound 3

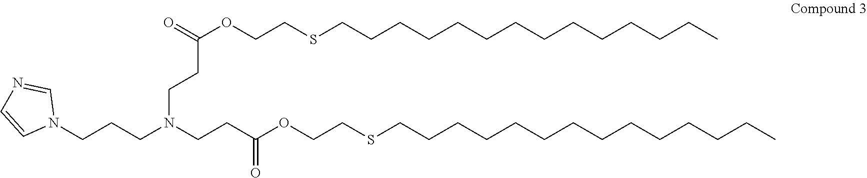 [SMILES: CCCCCCCCCCCCCCSCCOC(=O)CCN(CCCn1ccnc1)CCC(=O)OCCSCCCCCCCCCCCCCC]

Compound 3 was synthesized as reported in Angew. Chem. Int. Ed. 2020, 59, 20083-20089. Briefly, 1-bromotetradecane (5.0 g, 18 mmol) and potassium carbonate (3.6 g, 26 mmol) were added to a solution of 2-mercaptoethanol (1.1 g, 14 mmol) in acetonitrile. The reaction solution was stirred overnight at 40° C., filtered and concentrated. The pure product (2-(tetradecylthio)ethyl acrylate) was obtained after column chromatography purification on silica gel using n-hexane/ethyl acetate as mobile phase. Then, the purified product (4.2 g, 12.8 mmol) and triethylamine (TEA, 1.9 g, 19.2 mmol) were dissolved in anhydrous DCM. Acryloyl chloride (1.4 g, 15.4 mmol) was added dropwise at 0° C., and the reaction mixture was stirred overnight. After a second column chromatography purification, 2-(tetradecylthio)ethyl acrylate was obtained as a colorless oil. 3-(1H-imidazol-1-yl)propan-1-amine and 2-(tetradecylthio)ethyl acrylate were mixed at 1 to 2.4 molar ratio in Teflon-lined glass screw-top vials at 70° C. for 48 h. The crude products were purified using a Teledyne Isco Chromatography system using the mobile phase of methanol/DCM. The gradient volume ratio of methanol to DCM was 0 for 5 min, 5% for 10 min, 10% for 10 min, 20% for 10 min and 100% for 5 min.

### Example 7d: Synthesis of Compound 4

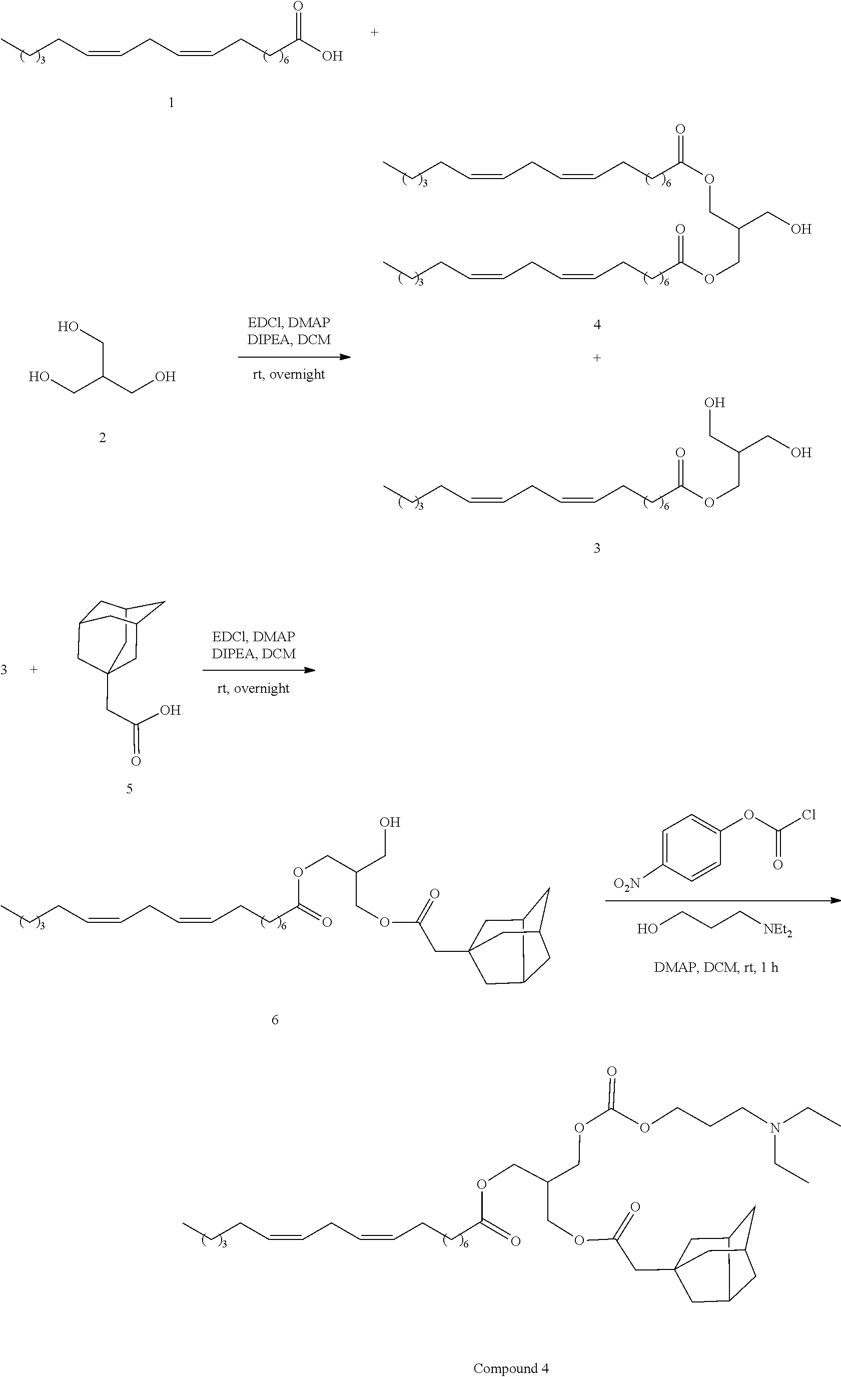

Compound 4 was synthesized as reported in Lokugamage, et al. Adv. Mater. 2019, 1902251. To a solution of linoleic acid (4.0 g, 14.2 mmol), 4-dimethylaminopyridine (DMAP) (0.4 g, 2.9 mmol), N, N-diisopropylethylamine (DIPEA) (3.7 mL, 20.5 mmol) and 2-(hydroxymethyl) propane-1,3-diol (1.5 g, 14.2 mmol) in anhydrous dichloromethane (40 mL) under nitrogen atmosphere was added N-(3-dimethylaminopropyl)-N′-ethylcarbodiimide hydrochloride (EDCl) (4.1 g, 20.5 mmol) at 25° C. The reaction mixture was stirred at room temperature overnight and linoleic acid was consumed completely, as determined by TLC, then the reaction mixture was directly concentrated under reduced pressure. Purification of the crude residue via silica gel flash column chromatography (gradient eluent: 1-30% of EtOAc/hexane) afforded intermediate 3 (2.3 g, 44% yield) and intermediate 4 (1.7 g, 30% yield) as colourless oil. To a solution of intermediate 3 (150 mg, 0.41 mmol), DMAP (10 mg, 0.1 mmol), DIPEA (0.1 ml, 0.6 mmol) and adamantane (79 mg, 0.41 mmol) in anhydrous dichloromethane (2 ml) under nitrogen atmosphere was added EDCI (114 mg, 0.6 mmol). The reaction mixture was stirred at room temperature for overnight. and intermediate 3 was consumed completely monitored by TLC, then the reaction mixture was directly concentrated under reduced pressure. Purification of the crude residue via silica gel flash column chromatography (gradient eluent: 0-20% of EtOAc/hexane) afforded intermediate 6 (103 mg, 53% yield) as colourless oil. To a solution of intermediate 6 (76 mg, 0.16 mmol) and DMAP (45 mg, 0.37 mmol) in anhydrous dichloromethane (2 ml) under nitrogen atmosphere was added 4-nitrophenylchloroformate (65 mg, 0.32 mmol). After stirring at room temperature for 1 hour, 3-diethylamino-1-propanol (0.44 ml, 0.96 mmol) was added into the reaction mixture and then stirred at room temperature for 1 hour. The reaction mixture was directly concentrated under reduced pressure. Purification of the crude residue via silica gel flash column chromatography (gradient eluent: 0-4% of MeOH/DCM) afforded compound 4 (32 mg, 29% yield) as colorless oil.

### Example 8: Preparation of Lipid Nanoparticles

Ionizable lipids, phospholipids, cholesterol, and PEG-lipids were dissolved in pure ethanol at the ratios listed in Table 8-1 with a total lipid concentration of 10.8 mM. See, e.g., Qiu et al., PNAS 118:e2020401118 (2021). The lipid solution was mixed at a 3:1 volume ratio with an acidic sodium acetate buffer (pH 5.0) or sodium citrate buffer (pH 4.0) containing mRNA (0.10 mg/mL) using the NanoAssemblr microfluidic system at a 12 mL/min total flow rate resulting in rapid mixing and self-assembly of LNPs. Formulations were further dialyzed against PBS (pH 7.4) overnight at 4° C. The particle size of formulations was measured by dynamic light scattering (DLS) using a Zetasizer Ultra (Malvern Panalytical). RNA encapsulation efficiency was determined by Ribogreen assay.

TABLE 8-1
Lipid nanoparticle formulations

| | | Phospho- | | | |
|---|---|---|---|---|---|
| Formulation | Ionizable Lipid | lipid | Cholesterol | PEG-Lipid | Buffer |
| F-1 | Compound 1 | DSPC | Cholesterol | C18PEG- | 50 mM |
| (molar ratio) | | | | DSPE | citrate |
| | (45:15:38.5:1.5) | | | | |
| F-2 | Compound 2 | DSPC | Cholesterol | C18PEG- | 50 mM |
| (molar ratio) | | | | DSPE | citrate |
| | (48.5:10:40:1.5) | | | | |
| F-3 | Compound 3 | DOPE | Cholesterol | C18PEG- | 25 mM |
| (molar ratio) | | | | DSPE | acetate |
| | (63:4.1:31.8:1.1) | | | | |
| F-4 | Compound 4 | DSPC | Cholesterol | C14PEG- | 50 mM |
| (molar ratio) | | | | DMPE | citrate |
| | (48.5:10:40:1.5) | | | | |
| F-5 | MC3* | DSPC | Cholesterol | C18PEG- | 50 mM |
| (molar ratio) | | | | DSPE | citrate |
| | (48.5:10:39.9:1.5) + 0.1 mol % ATTO-655^{#} | | | | |
| F-6 | Compound 1 | DSPC | Cholesterol | C18PEG- | 50 mM |
| (molar ratio) | | | | DSPE | citrate |
| | (45:15:38.4:1.5) + 0.1 mol % ATTO-655^{#} | | | | |
| F-7 | Compound 3 | DSPC | Cholesterol | C18PEG- | 25 mM |
| (molar ratio) | | | | DSPE | acetate |

(62.9:4.1:31.8:1.1) + 0.1 mol % ATTO-655^{#}

*MC3 = 4-(dimethylamino)-butanoic acid, (10Z,13Z)-1-(9Z,12Z)-9,12-octadecadien-1-yl-10,13-nonadecadien-1-yl ester;
^{#}ATTO-655 = Sigma Aldrich product no. 93711

TABLE 8-2
mRNA used in LNP formulations

SEQ ID NO: 1; linear mfLuc mRNA
AUGGAGGACGCCAAGAACAUCAAGAAGGGCCCCGCCCCCUUCUACCCCCUGGAGGACGGCACCGCCGGCGAGCAGC
UGCACAAGGCCAUGAAGCGGUACGCCCUGGUGCCCGGCACCAUCGCCUUCACCGACGCCCACAUCGAGGUGGACAU
CACCUACGCCGAGUACUUCGAGAUGAGCGUGCGGCUGGCCGAGGCCAUGAAGCGGUACGGCCUGAACACCAACCAC
CGGAUCGUGGUGUGCAGCGAGAACAGCCUGCAGUUCUUCAUGCCCGUGCUGGGCGCCCUGUUCAUCGGCGUGGCCG
UGGCCCCCGCCAACGACAUCUACAACGAGCGGGAGCUGCUGAACAGCAUGGGCAUCAGCCAGCCCACCGUGGUGUU
CGUGAGCAAGAAGGGCCUGCAGAAGAUCCUGAACGUGCAGAAGAAGCUGCCCAUCAUCCAGAAGAUCAUCAUCAUG
GACAGCAAGACCGACUACCAGGGCUUCCAGAGCAUGUACACCUUCGUGACCAGCCACCUGCCCCCCGGCUUCAACG
AGUACGACUUCGUGCCCGAGAGCUUCGACCGGGACAAGACCAUCGCCCUGAUCAUGAACAGCAGCGGCAGCACCGG
CCUGCCCAAGGGCGUGGCCCUGCCCCACCGGACCGCCUGCGUGCGGUUCAGCCACGCCCGGGACCCCAUCUUCGGC
AACCAGAUCAUCCCCGACACCGCCAUCCUGAGCGUGGUGCCCUUCCACCACGGCUUCGGCAUGUUCACCACCCUGG
GCUACCUGAUCUGCGGCUUCCGGGUGGUGCUGAUGUACCGGUUCGAGGAGGAGCUGUUCCUGCGGAGCCUGCAGGA
CUACAAGAUCCAGAGCGCCCUGCUGGUGCCCACCCUGUUCAGCUUCUUCGCCAAGAGCACCCUGAUCGACAAGUAC
GACCUGAGCAACCUGCACGAGAUCGCCAGCGGCGGCGCCCCCCUGAGCAAGGAGGUGGGCGAGGCCGUGGCCAAGC
GGUUCCACCUGCCCGGCAUCCGGCAGGGCUACGGCCUGACCGAGACCACCAGCGCCAUCCUGAUCACCCCCGAGGG
CGACGACAAGCCCGGCGCCGUGGGCAAGGUGGUGCCCUUCUUCGAGGCCAAGGUGGUGGACCUGGACACCGGCAAG
ACCCUGGGCGUGAACCAGCGGGGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGCCUACUGGGACGAGGACGAGCA
CUUCUUCAUCGUGGACCGGCUGAAGAGCCUGAUCAAGUACAAGGGCUACCAGGUGGCCCCCGCCGAGCUGGAGAGC
AUCCUGCUGCAGCACCCCAACAUCUUCGACGCCGGCGUGGCCGGCCUGCCCGACGACGACGCCGGCGAGCUGCCCG
CCGCCGUGGUGGUGCUGGAGCACGGCAAGACCAUGACCGAGAAGGAGAUCGUGGACUACGUGGCCAGCCAGGUGAC
CACCGCCAAGAAGCUGCGGGGCGGCGUGGUGUUCGUGGACGAGGUGCCCAAGGGCCUGACCGGCAAGCUGGACGCC
CGGAAGAUCCGGGAGAUCCUGAUCAAGGCCAAGAAGGGCGGCAAGAUCGCCGUGUGA
SEQ ID NO: 2; GFP mRNA
AUGGUGAGCAAGGGCGAGGAGCUGUUCACCGGGGUGGUGCCCAUCCUGGUCGAGCUGGACGGCGACGUAAACGGCC
ACAAGUUCAGCGUGUCCGGCGAGGGCGAGGGCGAUGCCACCUACGGCAAGCUGACCCUGAAGUUCAUCUGCACCAC
CGGCAAGCUGCCCGUGCCCUGGCCCACCCUCGUGACCACCCUGACCUACGGCGUGCAGUGCUUCAGCCGCUACCCC
GACCACAUGAAGCAGCACGACUUCUUCAAGUCCGCCAUGCCCGAAGGCUACGUCCAGGAGCGCACCAUCUUCUUCA
AGGACGACGGCAACUACAAGACCCGCGCCGAGGUGAAGUUCGAGGGCGACACCCUGGUGAACCGCAUCGAGCUGAA
GGGCAUCGACUUCAAGGAGGACGGCAACAUCCUGGGGCACAAGCUGGAGUACAACUACAACAGCCACAACGUCUAU
AUCAUGGCCGACAAGCAGAAGAACGGCAUCAAGGUGAACUUCAAGAUCCGCCACAACAUCGAGGACGGCAGCGUGC
AGCUCGCCGACCACUACCAGCAGAACACCCCCAUCGGCGACGGCCCCGUGCUGCUGCCCGACAACCACUACCUGAG
CACCCAGUCCGCCCUGAGCAAAGACCCCAACGAGAAGCGCGAUCACAUGGUCCUGCUGGAGUUCGUGACCGCCGCC
GGGAUCACUCUCGGCAUGGACGAGCUGUACAAGUAA

### Example 9: In Vivo FLuc mRNA Delivery and Bioluminescence Measurements

LNP/mRNA complexes were prepared as described in Example 8. BALB/c mice were injected via tail vein with 1.0 mg/kg FLuc mRNA (TriLink Biotechnologies) formulated in LNP formulations F-1, F-2, F-3 and F-4 in a total volume of 5 mL/kg. Each formulation was dosed in 6 mice, and an additional 6 mice were dosed with PBS control. 6 h and 24 hr of injection, 150 mg/kg D-luciferin was injected intraperitoneally and the whole-body bioluminescence signal was acquired ˜10 minutes after injection of D-luciferin using an IVIS Spectrum In Vivo Imaging System (PerkinElmer). Directly after whole body imaging, animals were immediately euthanized by CO₂ inhalation, and organs including liver, spleen, lung, heart and kidneys were harvested and subjected to bioluminescence imaging (BLI) analysis within 10 minutes of animal sacrifice. BLI images were detected in the auto-exposure mode. The BLI signal was quantitated using Living Image 4.7 software (Perkin Elmer) following the manufacturer's instruction. After BLI analysis the weights of the collected organs was measured. All in vivo experiments in this study were performed under the approved animal care guidelines.

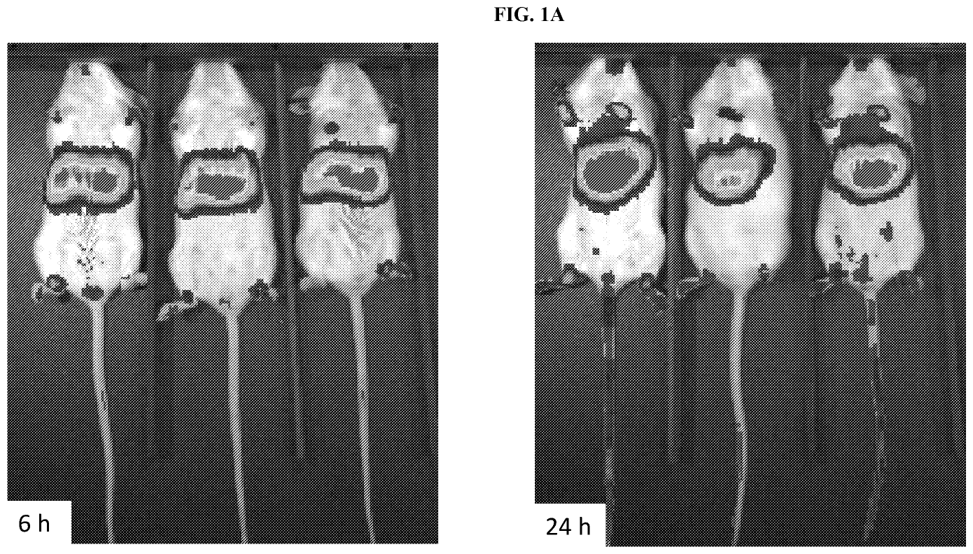

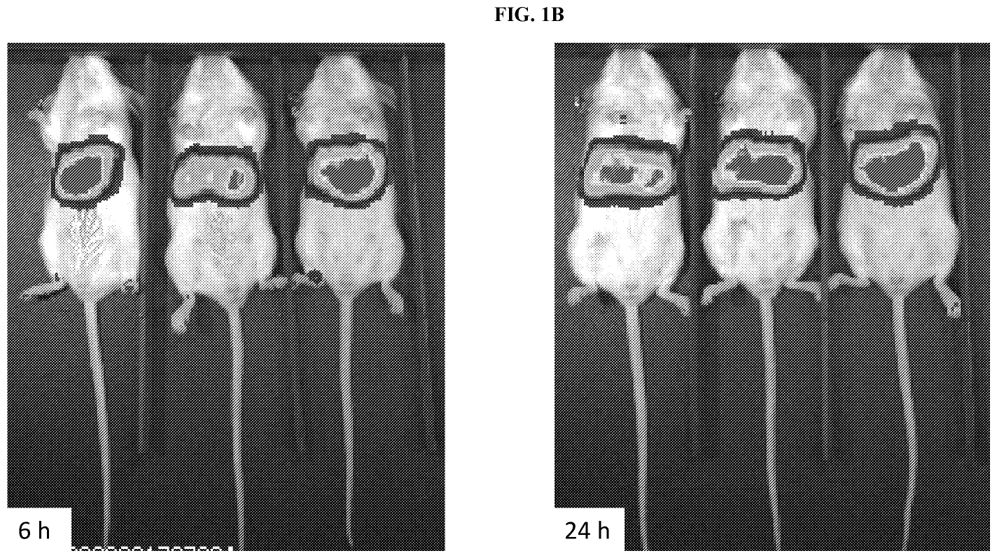

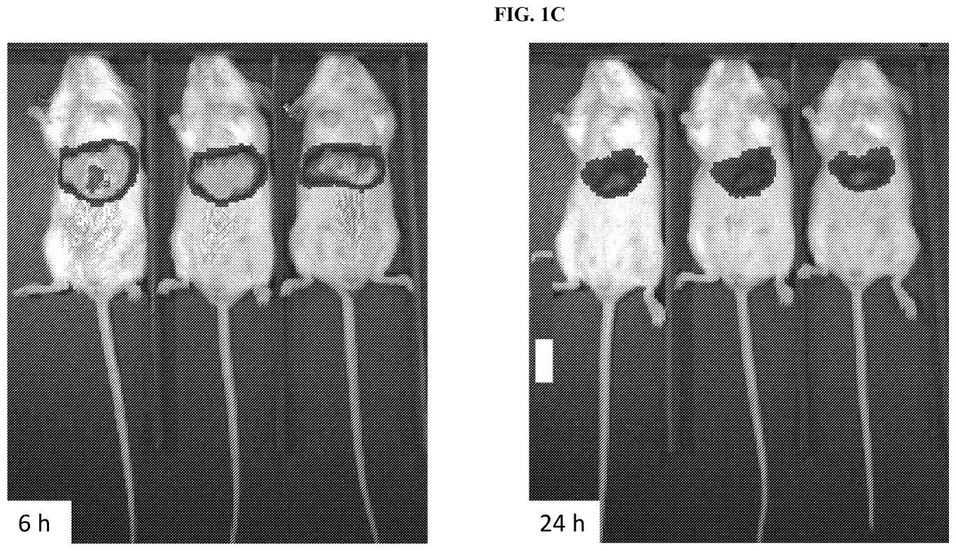

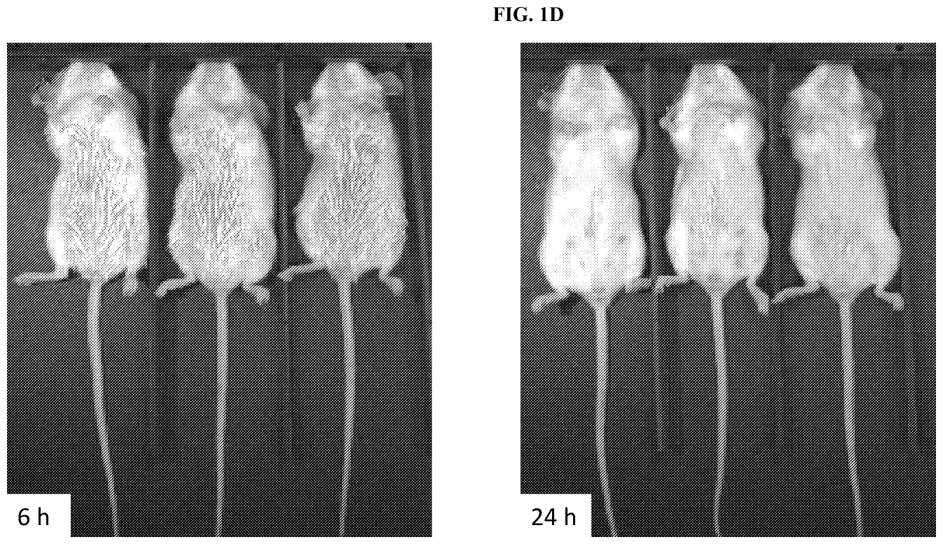

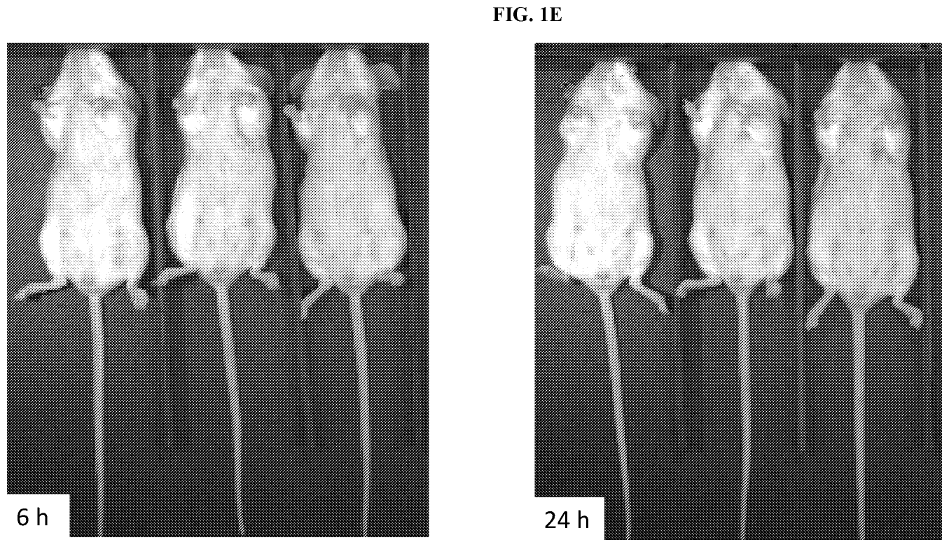

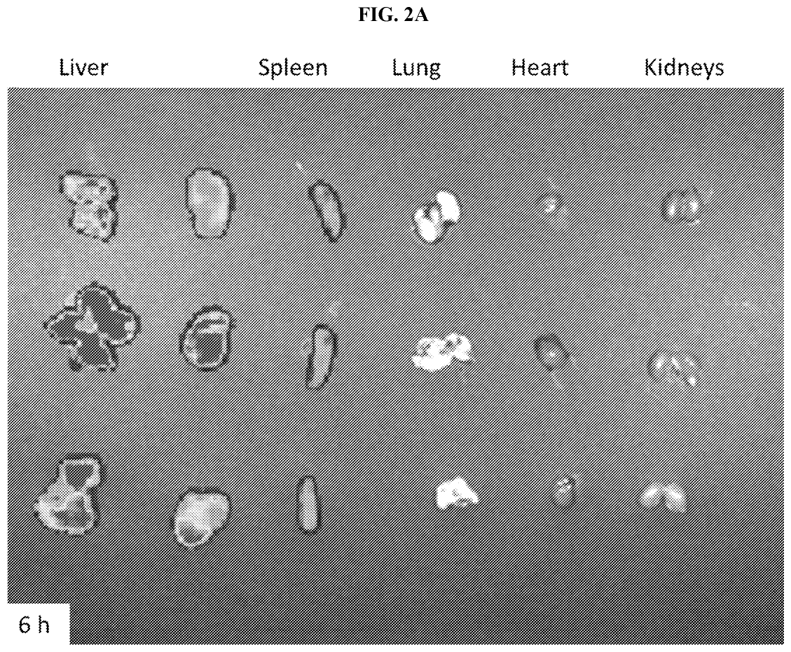

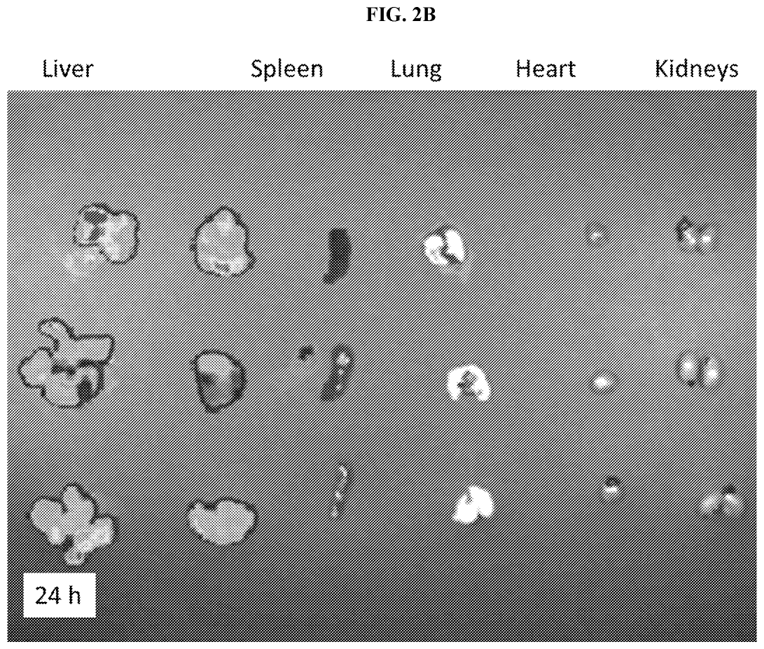

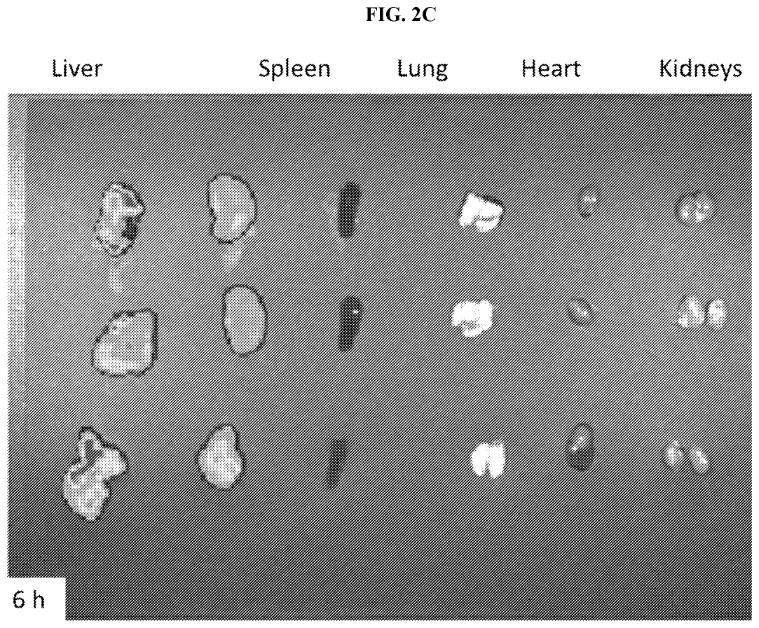

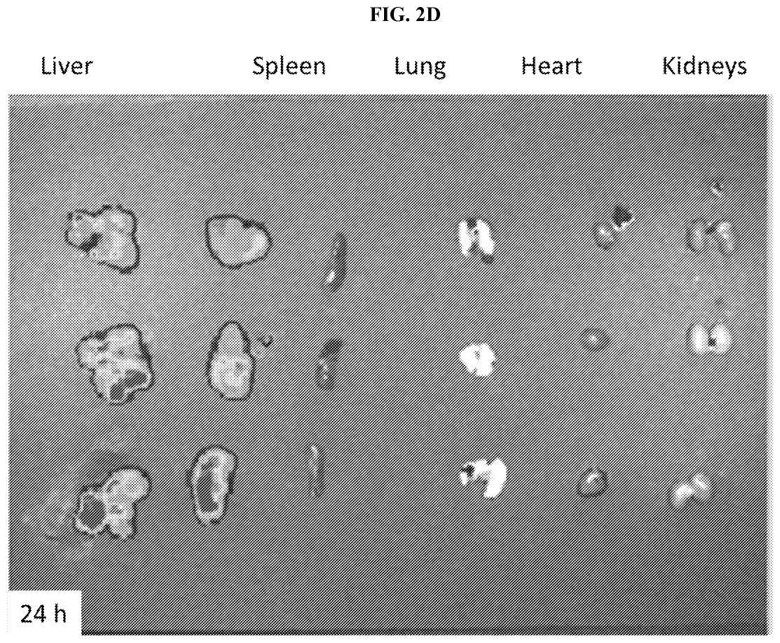

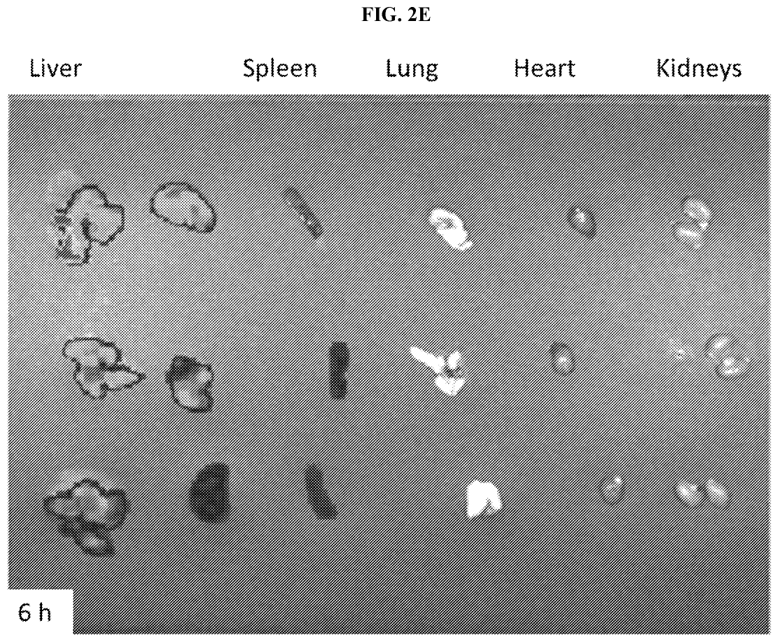

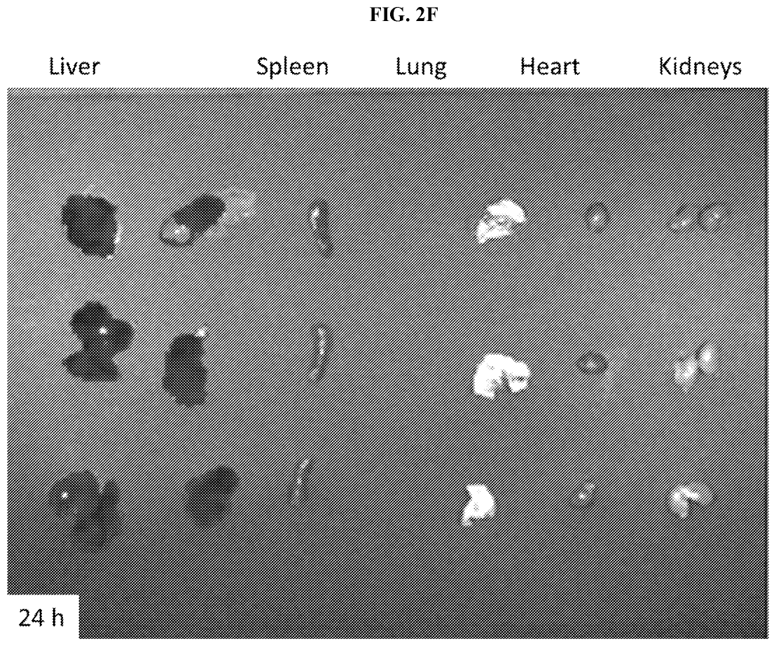

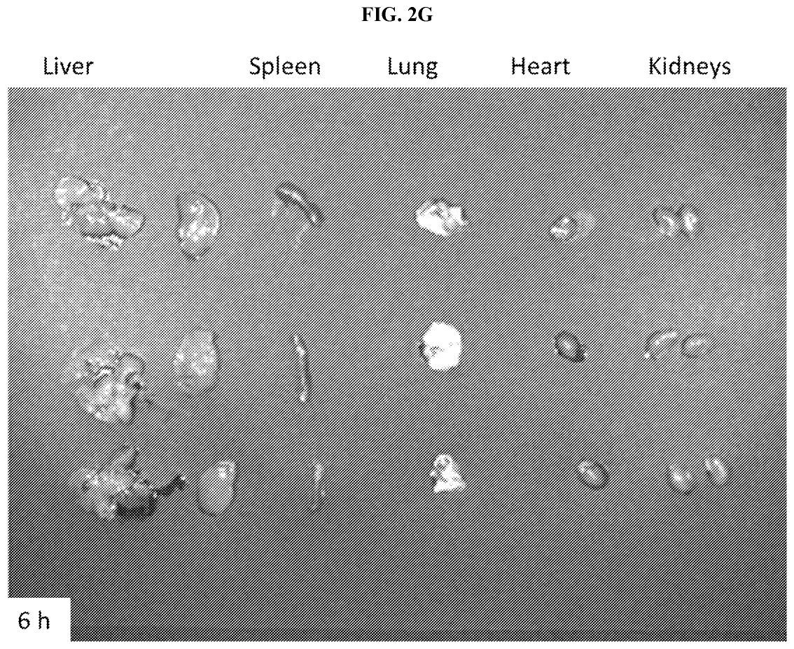

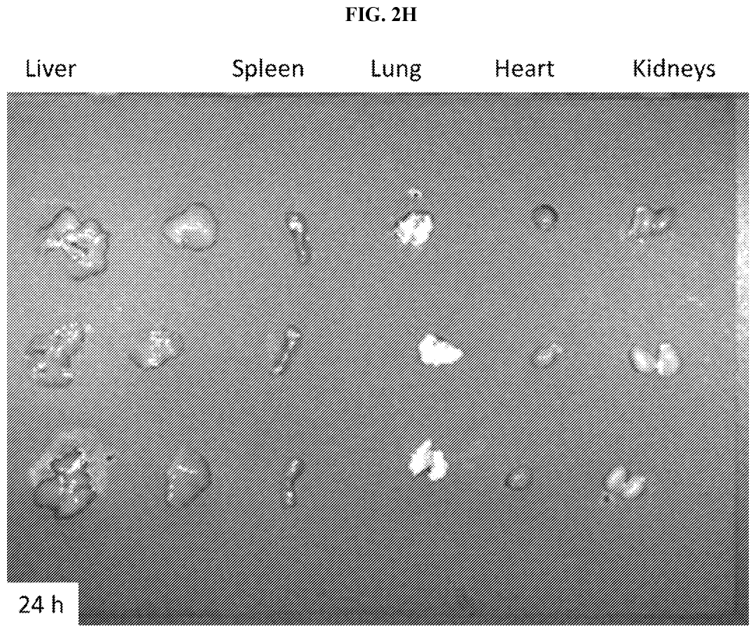

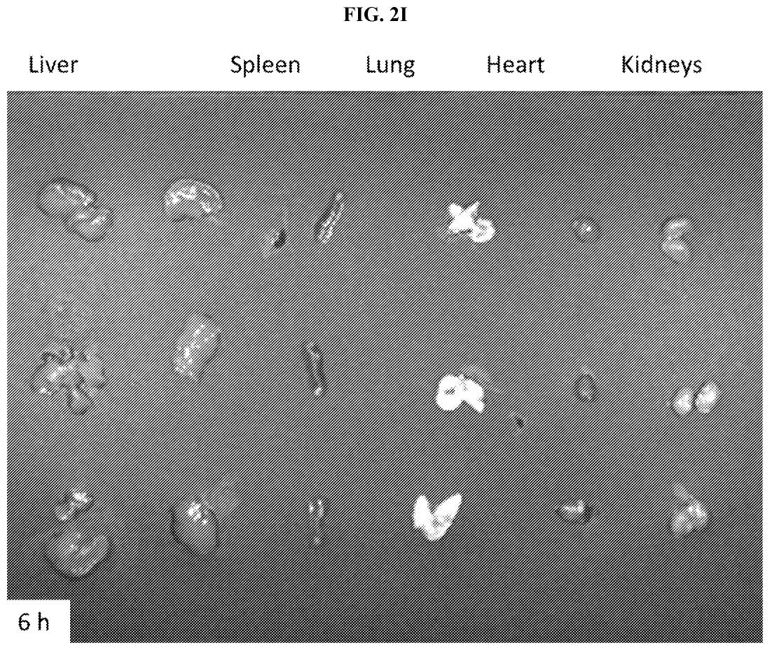

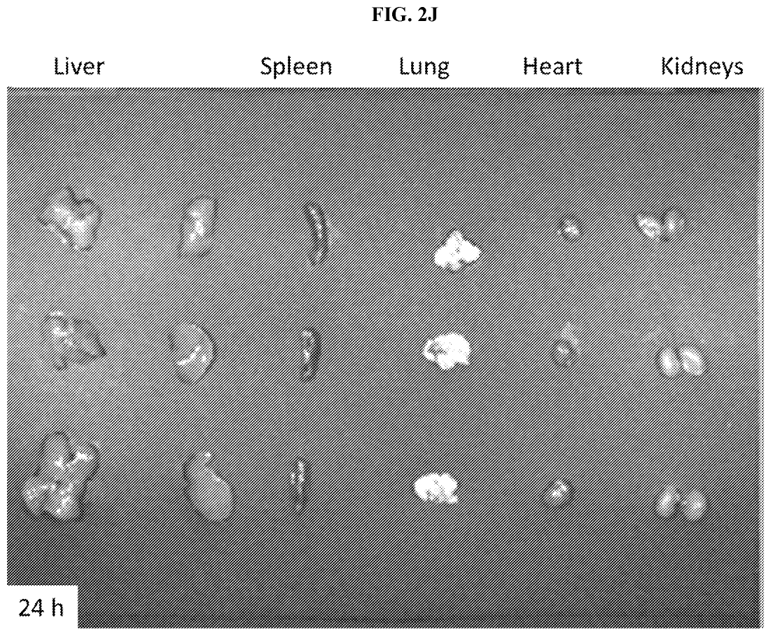

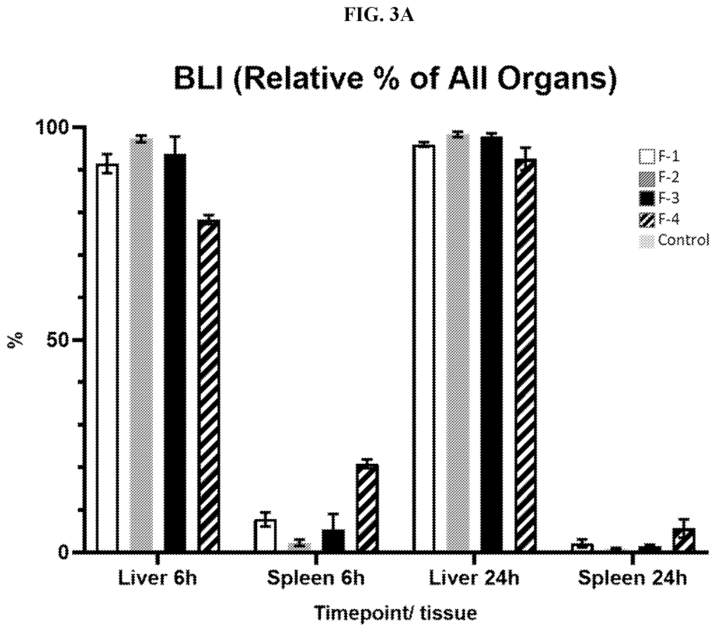

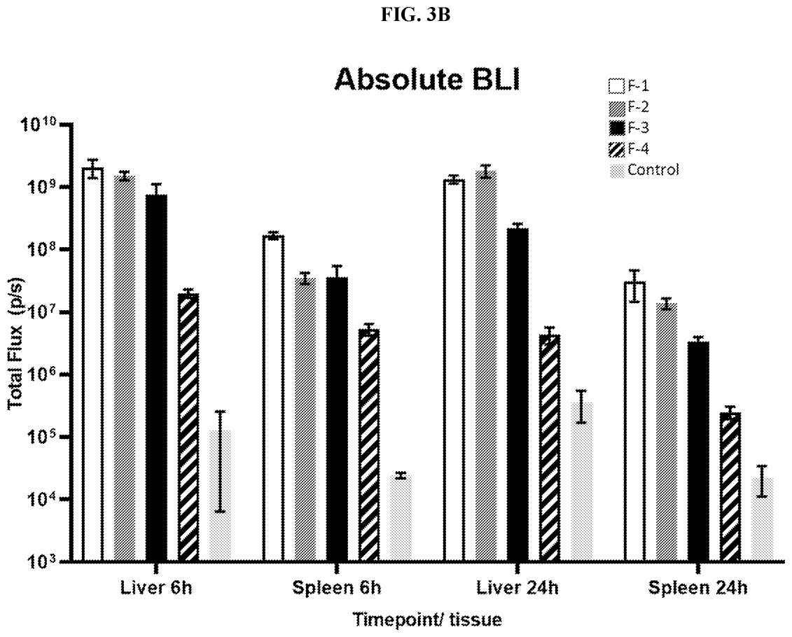

Full body animal images for animals sacrificed after 6 and 24 hours are shown in FIGS. 1A-1E. Individual organ images are shown in FIGS. 2A-2J. FIGS. 3A and 3B are bar graphs showing quantitative comparisons of the relative distribution of Formulations F-1, F-2, F-3 and F-4 between the liver and spleen of the mice after single IV administration of the fLuc mRNA LNPs, as determined via bioluminescence imagining. The averaged BLI in the mouse spleens as a percentage of all organs in the 6 hr mice were 21% for F-4, 6% for F-3, 8% for F-1, and 2% for F-2. The overall BLI intensity in spleen in the 6 hr mice were 5.4×10⁶ for F-4, 3.6×10⁷ for F-3, 1.7×10⁸ for F-1, and 3.5×10⁷ for F-2. These results suggest that LNP formulations F-1 and F-2 are superior, or at least comparable, for extrahepatic delivery of a cargo mRNA, as compared to published LNP formulations F-3 and F-4 which have been touted as extrahepatic delivery specialized LNP formulations (Lokugamage, et al. Adv. Mater. 2019, 1902251; Angew. Chem. Int. Ed. 2020, 59, 20083-20089).

### Example 10: Additional In Vivo FLuc mRNA Delivery and Bioluminescence Measurements

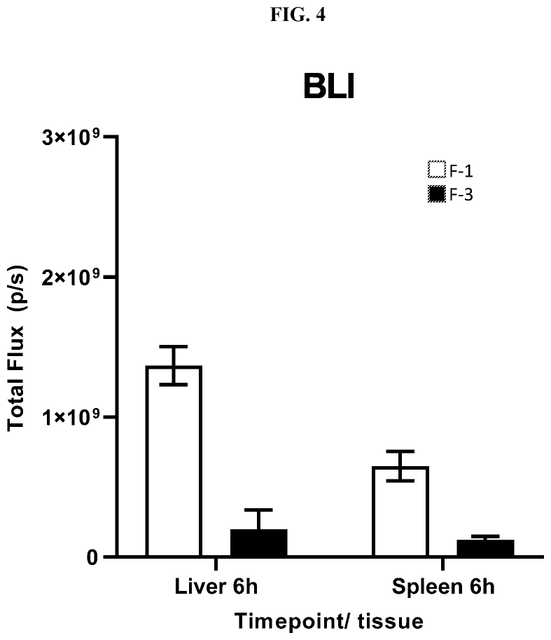

LNP/mRNA complexes were prepared as described in Example 8. BALB/c mice were injected via tail vein with 1.0 mg/kg FLuc mRNA (TriLink Biotechnologies) formulated in LNP formulations F-1 or F-3 in a total volume of 5 mL/kg. Each formulation was dosed in 3 mice, and an additional 2 mice were dosed with PBS control. 6 h post injection, 150 mg/kg D-luciferin was injected intraperitoneally and the whole-body bioluminescence signal was acquired ˜10 minutes after injection of D-luciferin using an IVIS Spectrum In Vivo Imaging System (PerkinElmer). Directly after whole body imaging, animals were immediately euthanized by CO₂ inhalation, and organs including liver, spleen, lung, heart and kidneys were harvested and subjected to bioluminescence imaging (BLI) analysis within 10 minutes of animal sacrifice. BLI images were collected and quantified as described in Example 9. FIG. 4 is a bar graph showing quantitative comparisons of the distribution of Formulations F-1 and F-3 between the liver and spleen of the mice after single IV administration of the fLuc mRNA LNPs, as determined via bioluminescence imagining. These results support the findings reported in Example 9 that LNP formulation F-1 is superior for extrahepatic delivery of a cargo mRNA, as compared to published extrahepatic delivery designed LNP formulation F-3 (Angew. Chem. Int. Ed. 2020, 59, 20083-20089).

### Example 11: Flow Cytometry Analysis of Splenocytes

LNP/mRNA complexes were prepared as described in Example 8. BALB/c mice were injected via tail vein with GFP mRNA (TriLink Biotechnologies) formulated in LNP formulations F-5 (2.0 mg/kg GFP mRNA), F-6 (1.5 mg/kg GFP mRNA) and F-7 (1.5 mg/kg GFP mRNA) in a total volume of 5 mL/kg. Each formulation was dosed in 3 mice, and an additional 2 mice were dosed with PBS control. 1 h post injection, animals were euthanized by CO₂ inhalation, and spleens were harvested. Harvested spleens were dissociated into single cell suspension of splenocytes by manually grinding the spleen over a 70 μm filter (Miltenyi 130-098-462) and washed with 1×PBS (ThermoFisher 10010049) containing 2 mM EDTA (ThermoFisher 15575-020) and 0.5% BSA (Miltenyi 130-091-376). Red blood cells were lysed using ACK Lysing Buffer (Thermo Fisher A1049201) and washed twice with 1×PBS+2 mM EDTA+0.5% BSA. Following final wash, cells were resuspended in 1×PBS and counted (ViCell XR, Beckman Coulter 731196). Cells were diluted, plated (5,000,000 per well) in a 96-well round bottom plate (Costar 3799), and stained for flow cytometry. Briefly, cells were stained in 1×PBS with Live/Dead Fixable Violet (Invitrogen L34964) at 1:1000 for 20 min at room temperature. Cells were then washed twice with Cell Staining Buffer (BioLegend 420201) and incubated with surface antibody stains either in full or FMO master mixes (panel and dilutions shown below in Table 11-1) for 30 min at 4° C. Cells were then washed twice with Cell Staining Buffer and fixed at 4° C. for 30 min IC Fixation Buffer (ThermoFisher 88-8824-00). Cells were washed twice with 1× permeabilization buffer (ThermoFisher 88-8824-00) and stained with anti-GFP antibody diluted 1:200 in permeabilization buffer overnight at 4° C. Cells were washed twice with 1× permeabilization buffer and resuspend in 1×PBS and acquired on cytometer (ThermoFisher Attune NXT with a laser configuration of Blue(3)/Red(3)/Violet(4)/Yellow(4)) equipped with a high-throughput autosampler (ThermoFisher CytKick). Compensation was performed using UltraComp eBeads (ThermoFisher 01-3333-41) and ArC Amine Reactive Compensation Bead Kit (ThermoFisher A10346).

TABLE 11-1
Surface antibody stains used in Example 11

| | | | | | | Usage per test |
|---|---|---|---|---|---|---|
| Staining Step | Target | Fluor | Clone | Manufacturer | Cat No. | (dilution; 1:X) |
| Live/Dead | Viability | Aqua | N/A | Invitrogen | L34966 | 1000 |
| Surface | Super Bright Staining Buffer | | | eBioscience | SB-4401-75 | 20 |
| | CD11b | PerCP/Cy5.5 | M1/70 | Biolegend | 101228 | 200 |
| | F4/80 | APC-Fire750 | BM8 | Biolegend | 123152 | 200 |
| | CD19 | BV421 | 6D5 | Biolegend | 115538 | 400 |
| | IA/IE | BV605 | M5/114.15.2 | Biolegend | 107639 | 500 |
| | CD8a | BV711 | 53-6.7 | Biolegend | 100759 | 200 |
| | CD3 | PE | 17A2 | Biolegend | 100206 | 200 |
| | CD11c | PE-eFluor610 | N418 | eBioscience | 61-0114-82 | 200 |
| | CD45 | PE/Cy7 | 30-F11 | Biolegend | 103114 | 200 |
| Intracellular | GFP | AF488 | FM264G | Biolegend | 338006 | 100 |

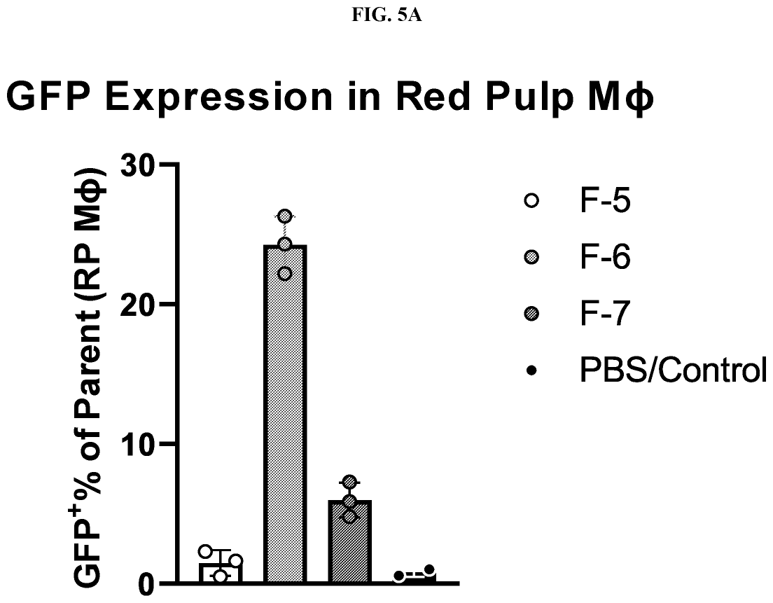

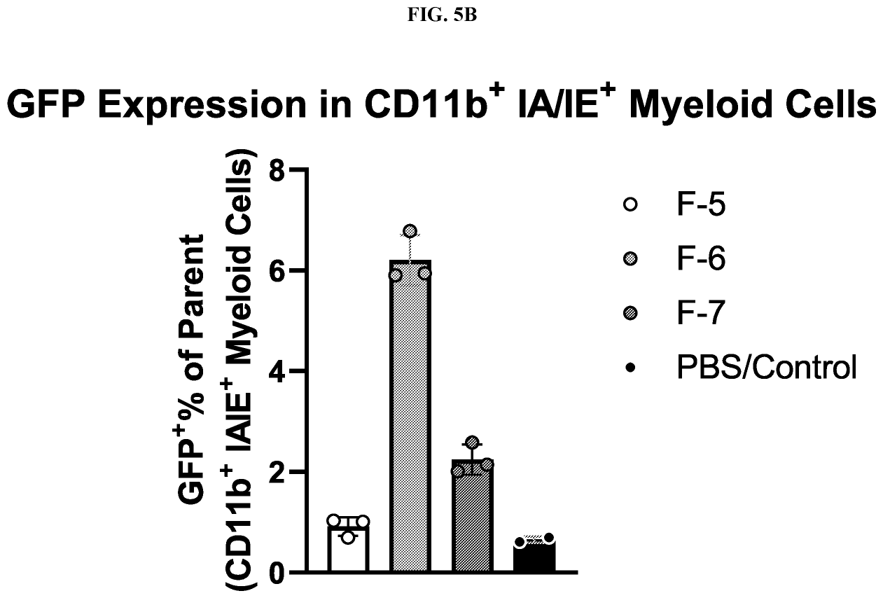

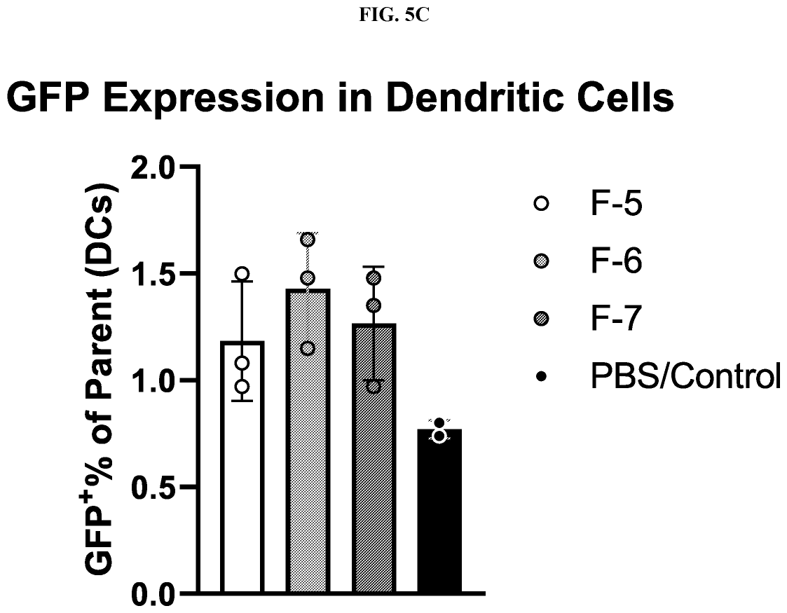

Of the spleen cell types examined, significant GFP expression was detected in red pulp macrophages, CD11b+ IA/E+ myeloid cells and dendritic cells. No substantial GFP expression was observed in B cells, or neutrophils. FIGS. 5A-5C are bar graphs showing GFP expression measured in red pulp macrophages, CD11b+ IA/E+ myeloid cells and dendritic cells 1 hour after administration. Formulation F-6, comprising ionizable lipid Compound 1, demonstrated the strongest GFP expression in the cells tested.

1. A pharmaceutical composition formulated for substantial extrahepatic delivery comprising:
a. a lipid nanoparticle comprising at least one ionizable lipid; and
b. a polynucleotide;
wherein at least about 5% of polynucleotide delivery occurs in a target organ that is not the liver, when the pharmaceutical composition is administered to a mammalian subject;
wherein the lipid nanoparticle encapsulates at least a portion of the polynucleotide and wherein the at least one ionizable lipid is selected from:
i) 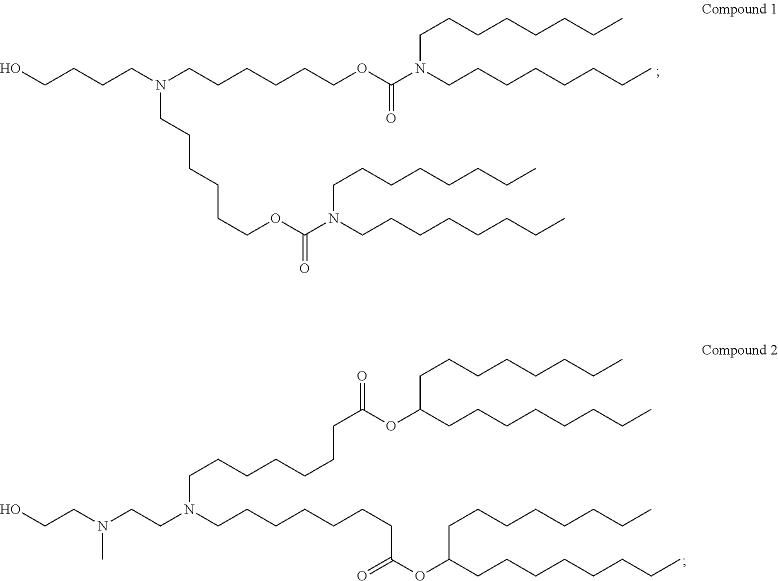 [SMILES: CCCCCCCCN(CCCCCCCC)C(=O)OCCCCCCN(CCCCO)CCCCCCOC(=O)N(CCCCCCCC)CCCCCCCC ; CCCCCCCCC(CCCCCCCC)OC(=O)CCCCCCCN(CCCCCCCC(=O)OC(CCCCCCCC)CCCCCCCC)CCN(C)CCO]
ii)
and
iii) an ionizable lipid disclosed in patent application publications WO2019/152557; WO2019/232095; WO2021/077067; WO2019/089828; US2019/0240354; US2010/0130588; US2021/0087135; US2021/0128488; US2020/0121809; US2013/0108685; US2013/0195920; US2015/0005363; US2014/0308304; US2017/0210697; and US2013/0053572.

2. The pharmaceutical composition of claim 1, wherein the lipid nanoparticle comprises Compound 1.

3. The pharmaceutical composition of claim 1, wherein the lipid nanoparticle comprises Compound 2.

4. The pharmaceutical composition of claim 1, wherein the lipid nanoparticle comprises an ionizable lipid disclosed in patent application publications WO2019/152557; WO2019/232095; WO2021/077067; WO2019/089828; US2019/0240354; US2010/0130588; US2021/0087135; US2021/0128488; US2020/0121809; US2013/0108685; US2013/0195920; US2015/0005363; US2014/0308304; US2017/0210697; and US2013/0053572.

5. The pharmaceutical composition of any one of claims 1-4, wherein the lipid nanoparticle delivers a higher proportion of the polynucleotide to the target organ than to the liver when administered to a mammalian subject.

6. The pharmaceutical composition of any one of claims 1-5, wherein the lipid nanoparticle delivers a higher proportion of the polynucleotide to a target organ than a reference lipid nanoparticle does when administered to a mammalian subject.

7. The pharmaceutical composition of claim 6, wherein the reference lipid nanoparticle comprises at least one ionizable lipid selected from Compound 3 and Compound 4:

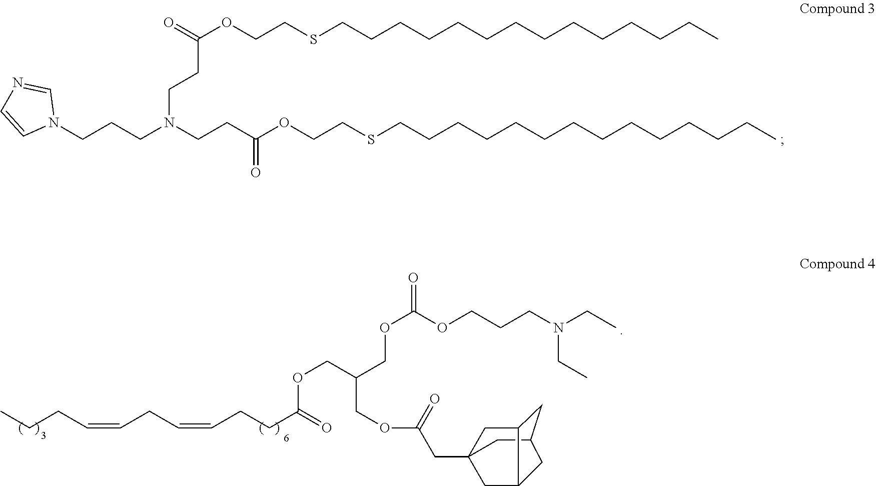

8. The pharmaceutical composition of claim 1, wherein the ionizable lipid has a head group listed on Table 1.

9. The pharmaceutical composition of claim 1, wherein the ionizable lipid has a head group that contains a short peptide of 12-15 mer length.

10. The pharmaceutical composition of claim 1, wherein the ionizable lipid has a head group that contains the structure of Vitamin A, D, E, or K.

11. The pharmaceutical composition of claim 1, wherein the ionizable lipid has a pKa between 6 and 7.

12. The pharmaceutical composition of claim 1, wherein the ionizable lipid is positively charged at pH 7.

13. The pharmaceutical composition of claim 11 or 12, wherein the target organ is the lung.

14. The pharmaceutical composition of any one of claims 1-13, wherein the lipid nanoparticle further comprises a PEGylated lipid.

15. The pharmaceutical composition of claim 14, wherein the PEGylated lipid is PEG-c-DOMG, PEG-DMG, PEG-DLPE, PEG-DMPE, PEG-DPPC, or PEG-DSPE.

16. The pharmaceutical composition of claim 14, wherein the PEGylated lipid is PEG-DMG.

17. The pharmaceutical composition of claim 14, wherein the PEGylated lipid is PEG-DSPE.

18. The pharmaceutical composition of any one of claims 1-17, wherein the lipid nanoparticle further comprises a structural lipid.

19. The pharmaceutical composition of claim 18, wherein the structural lipid is cholesterol.

20. The pharmaceutical composition of claim 18, wherein the structural lipid is a cholesterol analog.

21. The pharmaceutical composition of claim 18, wherein the structural lipid contains a plant sterol mimetic.

22. The pharmaceutical composition of any one of claims 1-21, wherein the lipid nanoparticle further comprises a phospholipid.

23. The pharmaceutical composition of claim 22, wherein the phospholipid is modified for enhanced endosomal escape.

24. The pharmaceutical composition of claim 22, wherein the phospholipid is selected from DOPE and DSPC.

25. The pharmaceutical composition of any one of claims 1-24, wherein the lipid nanoparticle further comprises at least one lipid selected from DDAB, EPC, 14PA, 18BMP, DODAP, DOTAP, and C12-200.

26. The pharmaceutical composition of any one of claims 1-25, wherein the polynucleotide is DNA.

27. The pharmaceutical composition of any one of claims 1-25, wherein the polynucleotide is RNA.

28. The pharmaceutical composition of claim 27, wherein the RNA is circular RNA.

29. The pharmaceutical composition of claim 27, wherein the RNA is a short interfering RNA (siRNA), an asymmetrical interfering RNA (aiRNA), a microRNA (miRNA), a Dicer-substrate RNA (dsRNA), or a short hairpin RNA (shRNA).

30. The pharmaceutical composition of claim 29, wherein the RNA consists of fewer than about 15 nucleotides.

31. The pharmaceutical composition of claim 29, wherein the RNA consists of fewer than about 50 nucleotides.

32. The pharmaceutical composition of any one of claims 27-31, wherein the polynucleotide encodes a protein.

33. The pharmaceutical composition of claim 27, wherein the polynucleotide comprises at least about 15 nucleotides.

34. The pharmaceutical composition of claim 27, wherein the polynucleotide comprises at least about 50 nucleotides.

35. The pharmaceutical composition of any one of claims 1-34, wherein the polynucleotide consists of natural nucleotides.

36. The pharmaceutical composition of claim 1, wherein the target organ is the kidney, placenta, heart, lung, muscle, fat, bladder, spleen, adrenal glands, brain, vagina, immune system, central nervous system, or skin.

37. The pharmaceutical composition of claim 1, wherein the target organ is the spleen.

38. The pharmaceutical composition of claim 1, wherein the target organ is the kidney.

39. The pharmaceutical composition of claim 1, wherein the target organ is the lung.

40. The pharmaceutical composition of claim 1, wherein the target organ is the heart.

41. The pharmaceutical composition of claim 1, wherein the target organ is the brain or central nervous system.

42. The pharmaceutical composition of claim 1, further comprising a target organ binding moiety operably connected to the lipid nanoparticle.

43. The pharmaceutical composition of any one of claims 1-42, wherein at least about 10% of polynucleotide delivery occurs in a target organ when administered to a mammalian subject.

44. The pharmaceutical composition of any one of claims 1-42, wherein at least about 15% of polynucleotide delivery occurs in a target organ when administered to a mammalian subject.

45. The pharmaceutical composition of any one of claims 1-42, wherein at least about 20% of polynucleotide delivery occurs in a target organ when administered to a mammalian subject.
